(19) United States
(12) Patent Application Publication
(10) Pub. No.: US 2024/0116955 A1
(43) Pub. Date: Apr. 11, 2024

(54) HETEROCYCLIC COMPOUND, ORGANIC LIGHT-EMITTING DEVICE INCLUDING THE SAME, AND DIAGNOSTIC COMPOSITION INCLUDING THE HETEROCYCLIC COMPOUND

(71) Applicant: Samsung Electronics Co., Ltd., Suwon-si (KR)

(72) Inventors: Eunkyung LEE, Suwon-si, Gyeonggi-do (US); Juhyun KIM, Suwon-si, Gyeonggi-do (KR); Sangmo KIM, Suwon-si, Gyeonggi-do (KR); Yusuke MARUYAMA, Suwon-si, Gyeonggi-do (KR); Yongsik JUNG, Suwon-si, Gyeonggi-do (KR); Jiwhan KIM, Suwon-si, Gyeonggi-do (KR); Youngmok Son, Suwon-si, Gyeonggi-do (KR); Sooghang Ihn, Suwon-si, Gyeonggi-do (KR); Jun CHWAE, Suwon-si, Gyeonggi-do (KR)

(21) Appl. No.: 18/456,826

(22) Filed: Aug. 28, 2023

(30) Foreign Application Priority Data
Aug. 29, 2022 (KR) 10-2022-0108725

(51) Int. Cl. C07F 5/02; C09K 11/06; H10K 50/12; H10K 85/60

(52) U.S. Cl. CPC C07F 5/02; C09K 11/06; H10K 50/121; H10K 85/658; C09K 2211/1074

(57) ABSTRACT

Provided are a heterocyclic compound represented by Formula 1, an organic light-emitting device including the same, and a diagnostic composition including the heterocyclic compound as described in the detailed description.

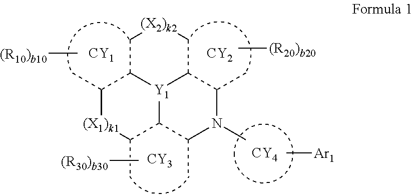

### CROSS-REFERENCE TO RELATED APPLICATION

This application claims priority under 35 U.S.C. § 119 to Korean Patent Application No. 10-2022-0108725, filed on Aug. 29, 2022, in the Korean Intellectual Property Office, the disclosure of which is incorporated by reference herein in its entirety.

### BACKGROUND

### 1. Field

The disclosure relates to a heterocyclic compound, an organic light-emitting device including the same, and a diagnostic composition including the heterocyclic compound.

### 2. Description of the Related Art

Organic light-emitting devices are self-emissive devices, which have improved characteristics in terms of viewing angles, response time, luminance, driving voltage, and response speed, and produce full-color images.

An organic light-emitting device may include an anode, a cathode, and an organic layer that is located between the anode and the cathode and includes an emission layer. A hole transport region may be arranged between the anode and the emission layer, and an electron transport region may be arranged between the emission layer and the cathode. Holes provided from the anode move toward the emission layer through the hole transport region, and electrons provided from the cathode move toward the emission layer through the electron transport region. The holes and the electrons recombine in the emission layer to produce excitons. These excitons transition from an excited state to the ground state to thereby generate light.

### SUMMARY

Provided are a heterocyclic compound, an organic light-emitting device including the same, and a diagnostic composition including the heterocyclic compound.

Additional aspects will be set forth in part in the description which follows and, in part, will be apparent from the description, or may be learned by practice of the presented embodiments of the disclosure.

According to one or more embodiments, there is provided a heterocyclic compound represented by Formula 1

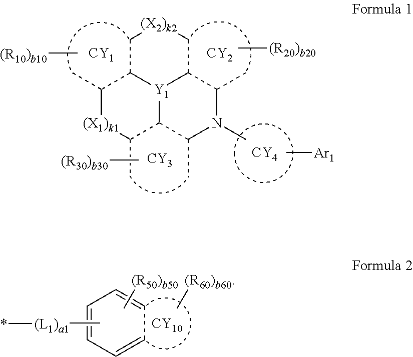

In Formulae 1 and 2,
Y₁ is B, N, P, P(═O), or P(═S),
X₁ and X₂ are each independently a single bond, O, S, Se, N[(CY₅)—Ar₂], N(R₁), C(R₁)(R₂), Si(R₁)(R₂), Ge(R₁)(R₂), B(R₁), P(R₁), P(═O)(R₁), S(═O)₂, or C(═O),
k1 is 0 or 1,
k2 is 0 or 1,
the sum of k1 and k2 is greater than or equal to 1,
CY₁ to CY₃ are each independently a C₅-C₃₀ carbocyclic group or a substituted or unsubstituted C₁-C₃₀ heterocyclic group,
CY₄ and CY₅ are each independently a C₅-C₃₀ carbocyclic group that is unsubstituted or substituted with at least one R₄₀ or a C₁-C₃₀ heterocyclic group that is unsubstituted or substituted with at least one R₄₀,
Ar₁ and Ar₂ are each independently a group represented by Formula 2,
L₁ is a single bond, a substituted or unsubstituted C₅-C₃₀ carbocyclic group, or a substituted or unsubstituted C₁-C₃₀ heterocyclic group,
a1 is 1, 2, or 3,
CY₁₀ is a C₃-C₁₀ non-aromatic carbocyclic group or a C₁-C₁₀ non-aromatic heterocyclic group,
R₁, R₂, R₁₀, R₂₀, R₃₀, and R₄₀ are each independently a group represented by Formula 2, hydrogen, deuterium, —F, —Cl, —Br, —I, —SF₅, a hydroxyl group, a cyano group, a nitro group, an amidino group, a hydrazino group, a hydrazono group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkylheteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₁)(Q₂)(Q₃), —Ge(Q₁)(Q₂)(Q₃), —C(═O)(Q₁), —S(═O)(Q₁), —S(═O)₂(Q₁), —B(Q₁)(Q₂), —P(Q₁)(Q₂), —P(═O)(Q₁)(Q₂), —P(═S)(Q₁)(Q₂), or a group represented by Formula 3,
R₅₀ and R₆₀ are each independently hydrogen, deuterium, —F, —Cl, —Br, —I, —SF₅, a hydroxyl group, a cyano group, a nitro group, an amidino group, a hydrazino group, a hydrazono group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkylheteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₁)(Q₂)(Q₃), —Ge(Q₁)(Q₂)(Q₃), —C(═O)(Q₁), —S(═O)(Q₁), —S(═O)₂(Q₁), —B(Q₁)(Q₂), —P(Q₁)(Q₂), —P(═O)(Q₁)(Q₂), —P(═S)(Q₁)(Q₂), or a group represented by Formula 3,

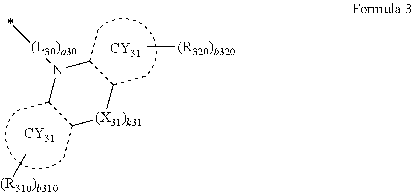

wherein, in Formula 3,
X₃₁ is a single bond, O, S, N(R₃₀₁), or C(R₃₀₁)(R₃₀₂),
k31 is 0 or 1,
CY₃₁ and CY₃₂ are each independently a C₅-C₃₀ carbocyclic group or a C₁-C₃₀ heterocyclic group,
L₃₀ is a single bond, a substituted or unsubstituted C₅-C₃₀ carbocyclic group, or a substituted or unsubstituted C₁-C₃₀ heterocyclic group,
a30 is 0, 1, 2, or 3,
R₃₀₁, R₃₀₂, R₃₁₀, and R₃₂₀ are each independently hydrogen, deuterium, —F, —Cl, —Br, —I, —SF₅, a hydroxyl group, a cyano group, a nitro group, an amidino group, a hydrazino group, a hydrazono group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkylheteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₁)(Q₂)(Q₃), —Ge(Q₁)(Q₂)(Q₃), —C(═O)(Q₁), —S(═O)(Q₁), —S(═O)₂(Q₁), —B(Q₁)(Q₂), —P(Q₁)(Q₂), —P(═O)(Q₁)(Q₂), or —P(═S)(Q₁)(Q₂),
b310 and b320 are each independently 1, 2, 3, 4, 5, 6, 7, or 8,
b10, b20, b30, b40, and b60 are each independently 1, 2, 3, 4, 5, 6, 7, or 8,
b50 is 1, 2, or 3,
a substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkylaryl group, the substituted C₆-C₆₀ aryloxy group, the substituted C₆-C₆₀ arylthio group, the substituted C₁-C₆₀ heteroaryl group, the substituted C₂-C₆₀ alkylheteroaryl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group is:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, or a C₁-C₆₀ alkoxy group,
a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₁)(Q₁₂)(Q₁₃), —Ge(Q₁₁)(Q₁₂)(Q₁₃), —C(═O)(Q₁₁), —S(═O)(Q₁₁), —S(═O)₂(Q₁₁), —B(Q₁₁)(Q₁₂), —P(Q₁₁)(Q₁₂), —P(═O)(Q₁₁)(Q₁₂), —P(═S)(Q₁₁)(Q₁₂), or any combination thereof,
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₁)(Q₂₂)(Q₂₃), —Ge(Q₂₁)(Q₂₂)(Q₂₃), —C(═O)(Q₂₁), —S(═O)(Q₂₁), —S(═O)₂(Q₂₁), —B(Q₂₁)(Q₂₂), —P(Q₂₁)(Q₂₂), —P(═O)(Q₂₁)(Q₂₂), —P(═S)(Q₂₁)(Q₂₂), or any combination thereof,
—N(Q₃₁)(Q₃₂), —Si(Q₃₁)(Q₃₂)(Q₃₃), —Ge(Q₃₁)(Q₃₂)(Q₃₃), —C(═O)(Q₃₁), —S(═O)(Q₃₁), —S(═O)₂(Q₃₁), —B(Q₃₁)(Q₃₂), —P(Q₃₁)(Q₃₂), —P(═O)(Q₃₁)(Q₃₂), or —P(Q₃₁)(Q₃₂),
Q₁ to Q₃, Q₁₁ to Q₁₃, Q₂₁ to Q₂₃, and Q₃₁ to Q₃₃ are each independently hydrogen, deuterium, —F, —Cl, —Br, —I, a hydroxyl group, a cyano group, a nitro group, an amidino group, a hydrazine group, a hydrazone group, a carboxylic acid or a salt thereof, a sulfonic acid or a salt thereof, a phosphoric acid or a salt thereof, a C₁-C₆₀ alkyl group that is unsubstituted or substituted with deuterium, a C₁-C₆₀ alkyl group, a C₆-C₆₀ aryl group, or any combination thereof, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group that is unsubstituted or substituted with deuterium, a C₁-C₆₀ alkyl group, a C₆-C₆₀ aryl group, or any combination thereof, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, and
* may indicate a binding site to a neighboring atom.

According to one or more embodiments, an organic light-emitting device includes the heterocyclic compound.

According to one or more embodiments, a diagnostic composition includes the heterocyclic compound.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a schematic cross-sectional view of an organic light-emitting device according to an exemplary embodiment;

FIG. 2 shows energy transfer of an organic light-emitting device according to an exemplary embodiment;

FIG. 3 shows energy transfer of an organic light-emitting device according to an exemplary embodiment;

FIG. 4 shows energy transfer of an organic light-emitting device according to an exemplary embodiment;

FIG. 5 shows energy transfer of an organic light-emitting device according to an exemplary embodiment; and

FIG. 6 shows energy transfer of an organic light-emitting device according to an exemplary embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly on the other element or intervening elements may be present therebetween In contrast, when an element is referred to as being “directly on” another element, there are no intervening elements present

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, “a,” “an,” “the,” and “at least one” do not denote a limitation of quantity, and are intended to cover both the singular and plural, unless the context clearly indicates otherwise. For example, “an element” has the same meaning as “at least one element,” unless the context clearly indicates otherwise.

“Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded Thus, the regions illustrated in the figures are schematic in nature and their shapes are not intended to illustrate the precise shape of a region and are not intended to limit the scope of the present claims.

A heterocyclic compound according to an embodiment is represented by Formula 1:

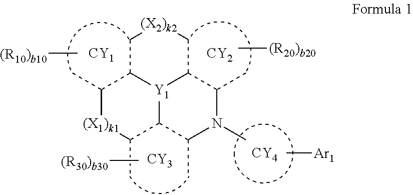

wherein, in Formula 1, Y₁ may be B, N, P, P(═O), or P(═S).

In an embodiment, Y₁ may be B, N, or P.

For example, Y₁ may be B.

In Formula 1, X₁ and X₂ may each independently be a single bond, O, S, Se, N[(CY₅)—Ar₂], N(R₁), C(R₁)(R₂), Si(R₁)(R₂), Ge(R₁)(R₂), B(R₁), P(R₁), P(═O)(R₁), S(═O)₂, or C(═O).

In an embodiment, X₁ and X₂ may each independently be a single bond, O, S, Se, N[(CY₅)—Ar₂], N(R₁), C(R₁)(R₂), or Si(R₁)(R₂).

In Formula 1, k1 may be 0 or 1. When k1 is 0, CY₁ and CY₃ are not linked to each other via X₁.

In Formula 1, k2 may be 0 or 1. When k2 is 0, CY₁ and CY₂ are not linked to each other via X₂.

In Formula 1, the sum of k1 and k2 may be greater than or equal to 1.

For example, at least one of k1 and k2 may be 1.

In Formula 1, CY₁ to CY₃ may each independently a C₅-C₃₀ carbocyclic group or a substituted or unsubstituted C₁-C₃₀ heterocyclic group.

In an embodiment, CY₁ to CY₃ may each independently be i) a first ring, ii) a second ring, iii) a condensed ring in which two or more first rings are condensed with each other, iv) a condensed ring in which two or more second rings are condensed with each other, or v) a condensed ring in which at least one first ring is condensed with at least one second ring.

The first ring may be a cyclopentane group, a cyclopentadiene group, a furan group, a thiophene group, a pyrrole group, a silole group, an indene group, a benzofuran group, a benzothiophene group, an indole group, a benzosilole group, an oxazole group, an isoxazole group, an oxadiazole group, an isoxadiazole group, an oxatriazole group, an isoxatriazole group, a thiazole group, an isothiazole group, a thiadiazole group, an isothiadiazole group, a thiatriazole group, an isothiatriazole group, a pyrazole group, an imidazole group, a triazole group, a tetrazole group, an azasilole group, a diazasilole group, or a triazasilole group, and
the second ring may be an adamantane group, a norbornene group, a bicyclo[1.1.1]pentane group, a bicyclo[2.1.1]hexane group, a bicyclo[2.2.1]heptane group (norbornane group), a bicyclo[2.2.2]octane group, a cyclohexane group, a cyclohexene group, a benzene group, a pyridine group, a pyrimidine group, a pyrazine group, a pyridazine group, or a triazine group.

In an embodiment, CY₁ to CY₃ may each independently be a C₆-C₃₀ aromatic carbocyclic group or a C₁-C₃₀ aromatic heterocyclic group.

In an embodiment, CY₁ to CY₃ may each independently be a benzene group, a naphthalene group, an anthracene group, a phenanthrene group, a triphenylene group, a pyrene group, a chrysene group, a 1,2,3,4-tetrahydronaphthalene group, a benzothiophene group, a benzofuran group, an indole group, an indene group, a benzosilole group, a benzoborole group, a benzophosphole group, a benzoselenophene group, a benzogermole group, a dibenzothiophene group, a dibenzofuran group, a carbazole group, a fluorene group, a dibenzosilole group, a dibenzoborole group, a dibenzophosphole group, a dibenzoselenophene group, a dibenzogermole group, a dibenzothiophene 5-oxide group, a 9H-fluoren-9-one group, a dibenzothiophene 5,5-dioxide group, an azabenzothiophene group, an azabenzofuran group, an azaindole group, an azaindene group, an azabenzosilole group, an azabenzoborole group, an azabenzophosphole group, an azabenzoselenophene group, an azabenzogermole group, an azadibenzothiophene group, an azadibenzofuran group, an azacarbazole group, an azafluorene group, an azadibenzosilole group, an azadibenzoborole group, an azadibenzophosphole group, an azadibenzoselenophene group, an azadibenzogermole group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a 5,6,7,8-tetrahydroisoquinoline group, a 5,6,7,8-tetrahydroquinoline group, adamantane group, a norbornane group, or a norbornene group.

In an embodiment, CY₁ to CY₃ may each independently be a benzene group, a naphthalene group, a phenanthrene group, a pyridine group, a pyrimidine group, a quinoline group, an isoquinoline group, a phthalazine group, a naphthyridine group, a quinoxaline group, or a quinazoline group.

In Formula 1, CY₄ and CY₅ may each independently be a C₅-C₃₀ carbocyclic group that is unsubstituted or substituted with at least one R₄₀ or a C₁-C₃₀ heterocyclic group that is unsubstituted or substituted with at least one R₄₀.

In an embodiment, CY₄ and CY₅ may each independently be a benzene group, a naphthalene group, an anthracene group, a phenanthrene group, a triphenylene group, a pyrene group, a chrysene group, a 1,2,3,4-tetrahydronaphthalene group, a benzothiophene group, a benzofuran group, an indole group, an indene group, a benzosilole group, a benzoborole group, a benzophosphole group, a benzoselenophene group, a benzogermole group, a dibenzothiophene group, a dibenzofuran group, a carbazole group, a fluorene group, a dibenzosilole group, a dibenzoborole group, a dibenzophosphole group, a dibenzoselenophene group, a dibenzogermole group, a dibenzothiophene 5-oxide group, a 9H-fluoren-9-one group, a dibenzothiophene 5,5-dioxide group, an azabenzothiophene group, an azabenzofuran group, an azaindole group, an azaindene group, an azabenzosilole group, an azabenzoborole group, an azabenzophosphole group, an azabenzoselenophene group, an azabenzogermole group, an azadibenzothiophene group, an azadibenzofuran group, an azacarbazole group, an azafluorene group, an azadibenzosilole group, an azadibenzoborole group, an azadibenzophosphole group, an azadibenzoselenophene group, an azadibenzogermole group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a 5,6,7,8-tetrahydroisoquinoline group, a 5,6,7,8-tetrahydroquinoline group, an adamantane group, a norbornane group, or a norbornene group.

In an embodiment, CY₄ and CY₅ may each independently be a benzene group, a naphthalene group, a phenanthrene group, a pyridine group, a pyrimidine group, a quinoline group, an isoquinoline group, a phthalazine group, a naphthyridine group, a quinoxaline group, or a quinazoline group, each unsubstituted or substituted with at least one R₄₀.

In an embodiment, CY₄ and CY₅ may each independently be a benzene group that is unsubstituted or substituted with at least one R₄₀.

In an embodiment, CY₄ and CY₅ may each independently be of Formulae 4-1 to 4-3:

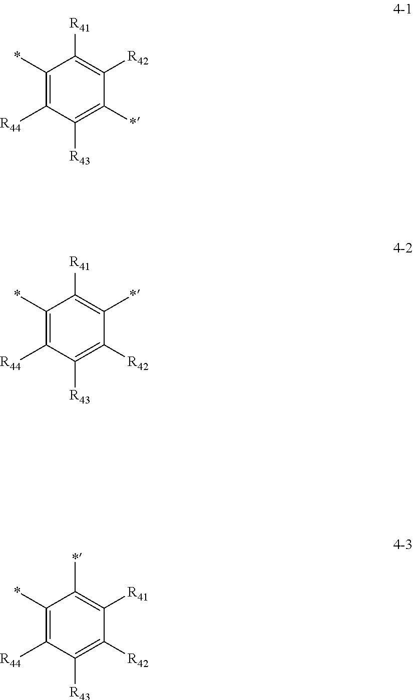

wherein, in Formulae 4-1 to 4-3,
R₄₁ to R₄₄ may each independently be the same as described in connection with R₄₀, and
* and *′ may each indicate a binding site to a neighboring atom.

In Formula 1, Ar₁ and Ar₂ may each independently be a group represented by Formula 2.

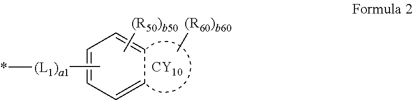

In Formula 2, L₁ may be a single bond, a substituted or unsubstituted C₅-C₃₀ carbocyclic group or a substituted or unsubstituted C₁-C₃₀ heterocyclic group.

In an embodiment, L₁ may be a single bond.

In Formula 2, a1 may be 1, 2, or 3.

In an embodiment, a1 may be 1.

In Formula 2, CY₁₀ may be a C₃-C₁₀ non-aromatic carbocyclic group or a C₁-C₁₀ non-aromatic heterocyclic group.

In an embodiment, CY₁₀ may be a cyclopentane group, a cyclohexane group, a cycloheptane group, a cyclopentene group, a cyclohexene group, or a cycloheptene group.

In Formula 2, * may indicate a binding site to a neighboring atom.

In an embodiment, Ar₁ and Ar₂ may each independently be a group represented by Formula 2A or 2B.

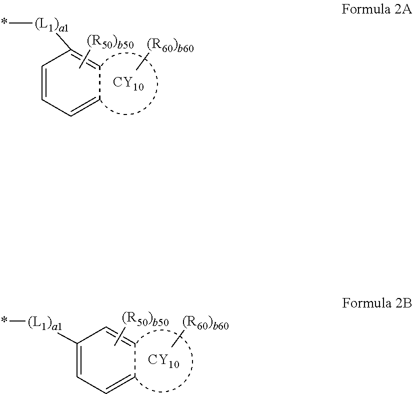

wherein, in Formulae 2A and 2B,
L₁, a1, CY₁₀, R₅₀, R₆₀, b50, and b60 may each be the same as described in the present specification, and
* may indicate a binding site to a neighboring atom.

In an embodiment, Ar₁ and Ar₂ may each independently be a group represented by one of Formulae 5-1 to 5-4:

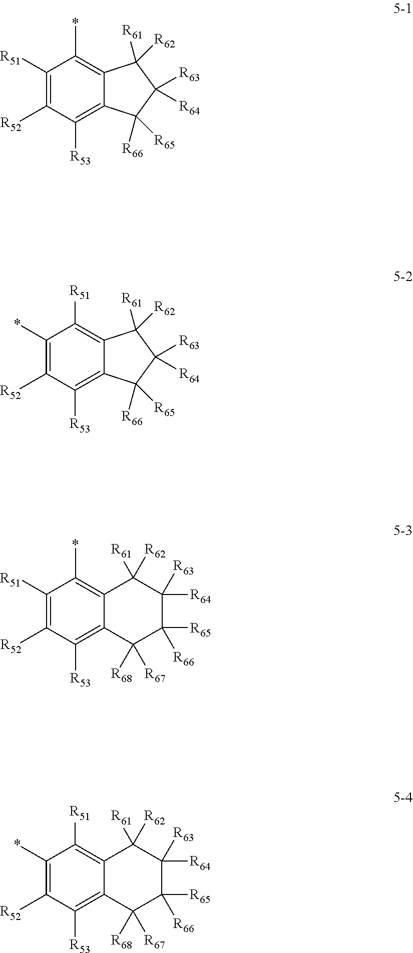

wherein, in Formulae 5-1 to 5-4,
R₅₁ to R₅₃ may each be the same as described in connection with R₅₀,
R₆₁ to R₆₈ may each be the same as described in connection with R₆₀, and
* may indicate a binding site to a neighboring atom.

In an embodiment, Ar₁ and Ar₂ may each independently be a group represented by one of Formulae 5-11 to 5-26:

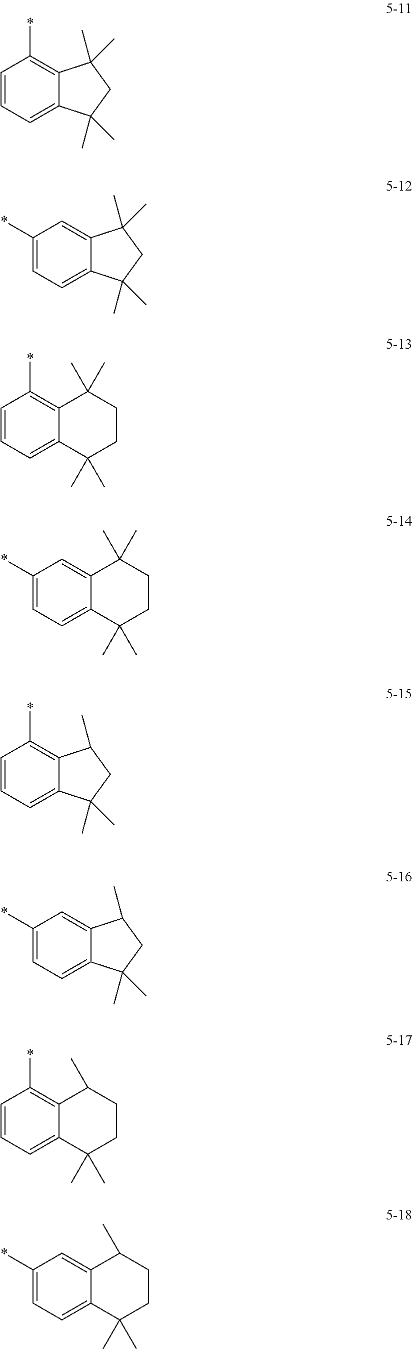

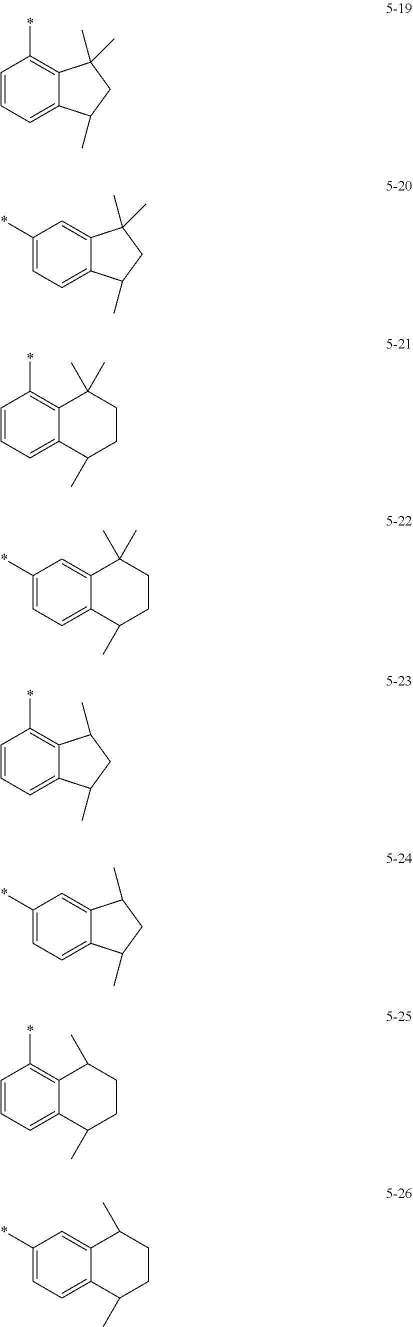

wherein, in Formulae 5-11 to 5-26,
* may indicate a binding site to a neighboring atom.

In an embodiment, Ar₁ and Ar₂ may have the same structure.

In an embodiment, Ar₁ and Ar₂ may have different structures.

In Formula 1, R₁, R₂, R₁₀, R₂₀, R₃₀, and R₄₀ may each independently be a group represented by Formula 2, hydrogen, deuterium, —F, —Cl, —Br, —I, —SF₅, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkylheteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₁)(Q₂)(Q₃), —Ge(Q₁)(Q₂)(Q₃), —C(═O)(Q₁), —S(═O)(Q₁), —S(═O)₂(Q₁), —B(Q₁)(Q₂), —P(Q₁)(Q₂), —P(═O)(Q₁)(Q₂), —P(═S)(Q₁)(Q₂), or a group represented by Formula 3.

In Formula 2, R₅₀ and R₆₀ may each independently be hydrogen, deuterium, —F, —Cl, —Br, —I, —SF₅, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkylheteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₁)(Q₂)(Q₃), —Ge(Q₁)(Q₂)(Q₃), —C(═O)(Q₁), —S(═O)(Q₁), —S(═O)₂(Q₁), —B(Q₁)(Q₂), —P(Q₁)(Q₂), —P(═O)(Q₁)(Q₂), —P(═S)(Q₁)(Q₂), or a group represented by Formula 3:

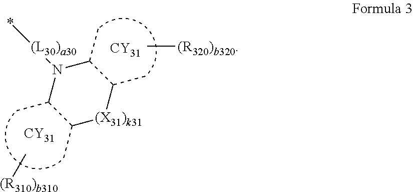

In Formula 3, X₃₁ may be a single bond, O, S, N(R₃₀₁), or C(R₃₀₁)(R₃₀₂).

In Formula 3, k31 may be 0 or 1.

In Formula 3, CY₃₁ and CY₃₂ may each independently be a C₅-C₃₀ carbocyclic group or a C₁-C₃₀ heterocyclic group.

In an embodiment, CY₃₁ and CY₃₂ may each independently be a benzene group, a naphthalene group, a 1,2,3,4-tetrahydronaphthalene group, a thiophene group, a furan group, a pyrrole group, a cyclopentadiene group, a silole group, a benzothiophene group, a benzofuran group, an indole group, an indene group, a benzosilole group, a dibenzothiophene group, a dibenzofuran group, a carbazole group, a fluorene group, or a dibenzosilole group.

In Formula 3, L₃₀ may be a single bond, a substituted or unsubstituted C₅-C₃₀ carbocyclic group, or a substituted or unsubstituted C₁-C₃₀ heterocyclic group.

In Formula 3, a30 may be 0, 1, 2, or 3.

In an embodiment, a30 may be 0 or 1.

In Formula 3, R₃₀₁, R₃₀₂, R₃₁₀, and R₃₂₀ may each independently be hydrogen, deuterium, —F, —Cl, —Br, —I, —SF₅, a hydroxyl group, a cyano group, a nitro group, an amidino group, a hydrazino group, a hydrazono group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkylheteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₁)(Q₂)(Q₃), —Ge(Q₁)(Q₂)(Q₃), —C(═O)(Q₁), —S(═O)(Q₁), —S(═O)₂(Q₁), —B(Q₁)(Q₂), —P(Q₁)(Q₂), —P(═O)(Q₁)(Q₂), or —P(═S)(Q₁)(Q₂).

In an embodiment, R₃₀₁, R₃₀₂, R₃₁₀, and R₃₂₀ may each independently be:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, or a C₁-C₂₀ alkoxy group;
a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a deuterium-containing C₁-C₂₀ alkyl group, a fluorinated C₁-C₂₀ alkyl group, a cyclopentyl group, a cyclohexyl group, a cycloheptyl group, a cyclooctyl group, an adamantanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group (a norbornanyl group), a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a silolanyl group, a phenyl group, a (C₁-C₂₀ alkyl)phenyl group, a biphenyl group, a terphenyl group, a naphthyl group, a 1,2,3,4-tetrahydronaphthyl group, a pyridinyl group, a pyrimidinyl group, or any combination thereof,
a cyclopentyl group, a cyclohexyl group, a cycloheptyl group, a cyclooctyl group, an adamantanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a silolanyl group, a phenyl group, a (C₁-C₂₀ alkyl)phenyl group, a biphenyl group, a terphenyl group, a naphthyl group, a 1,2,3,4-tetrahydronaphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, or an azadibenzothiophenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a deuterium-containing C₁-C₂₀ alkyl group, a fluorinated C₁-C₂₀ alkyl group, a C₁-C₂₀ alkoxy group, a cyclopentyl group, a cyclohexyl group, a cycloheptyl group, a cyclooctyl group, an adamantanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a silolanyl group, a phenyl group, a (C₁-C₂₀ alkyl)phenyl group, a biphenyl group, a terphenyl group, a naphthyl group, a 1,2,3,4-tetrahydronaphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, or any combination thereof; or
—N(Q₁)(Q₂), —Si(Q₁)(Q₂)(Q₃), —Ge(Q₁)(Q₂)(Q₃), —C(═O)(Q₁), —S(═O)(Q₁), —S(═O)₂(Q₁), —B(Q₁)(Q₂), —P(Q₁)(Q₂), —P(═O)(Q₁)(Q₂), or —P(═S)(Q₁)(Q₂).

In an embodiment, R₃₀₁, R₃₀₂, R₃₁₀, and R₃₂₀ may each independently be
hydrogen, deuterium, a methyl group, an ethyl group, an n-propyl group, an iso-propyl group, an n-butyl group, an isobutyl group, a sec-butyl group, a tert-butyl group, an n-pentyl group, an isopentyl group, a 2-methylbutyl group, a sec-pentyl group, a tert-pentyl group, a neo-pentyl group, a 3-pentyl group, a 3-methyl-2-butyl group, a phenyl group, a biphenyl group, a C₁-C₂₀ alkylphenyl group, or a naphthyl group.

In Formula 3, b310 and b320 may each independently be 1, 2, 3, 4, 5, 6, 7, or 8.

In an embodiment, R₁, R₂, R₁₀, R₂₀, R₃₀, and R₄₀ may each independently be: a group represented by Formula 2, a group represented by Formula 3, hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, or a C₁-C₂₀ alkoxy group;
a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a deuterium-containing C₁-C₂₀ alkyl group, a fluorinated C₁-C₂₀ alkyl group, a cyclopentyl group, a cyclohexyl group, a cycloheptyl group, a cyclooctyl group, an adamantanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group (a norbornanyl group), a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a silolanyl group, a phenyl group, a (C₁-C₂₀ alkyl)phenyl group, a biphenyl group, a terphenyl group, a naphthyl group, a 1,2,3,4-tetrahydronaphthyl group, a pyridinyl group, a pyrimidinyl group, or any combination thereof;
a cyclopentyl group, a cyclohexyl group, a cycloheptyl group, a cyclooctyl group, an adamantanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a silolanyl group, a phenyl group, a (C₁-C₂₀ alkyl)phenyl group, a biphenyl group, a terphenyl group, a naphthyl group, a 1,2,3,4-tetrahydronaphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, or an azadibenzothiophenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a deuterium-containing C₁-C₂₀ alkyl group, a fluorinated C₁-C₂₀ alkyl group, a C₁-C₂₀ alkoxy group, a cyclopentyl group, a cyclohexyl group, a cycloheptyl group, a cyclooctyl group, an adamantanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a silolanyl group, a phenyl group, a (C₁-C₂₀ alkyl)phenyl group, a biphenyl group, a terphenyl group, a naphthyl group, a 1,2,3,4-tetrahydronaphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, or any combination thereof; or

—N(Q₁)(Q₂), —Si(Q₁)(Q₂)(Q₃), —Ge(Q₁)(Q₂)(Q₃), —C(═O)(Q₁), —S(═O)(Q₁), —S(═O)₂(Q₁), —B(Q₁)(Q₂), —P(Q₁)(Q₂), —P(═O)(Q₁)(Q₂), —P(═S)(Q₁)(Q₂), or any combination thereof.

In an embodiment, R₅₀ and R₆₀ may each independently be: a group represented by Formula 3, hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, or a C₁-C₂₀ alkoxy group;
a C₁-C₂₀ alkyl group or a C₁-C₂₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a deuterium-containing C₁-C₂₀ alkyl group, a fluorinated C₁-C₂₀ alkyl group, a cyclopentyl group, a cyclohexyl group, a cycloheptyl group, a cyclooctyl group, an adamantanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group (a norbornanyl group), a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a silolanyl group, a phenyl group, a (C₁-C₂₀ alkyl)phenyl group, a biphenyl group, a terphenyl group, a naphthyl group, a 1,2,3,4-tetrahydronaphthyl group, a pyridinyl group, a pyrimidinyl group, or any combination thereof,
a cyclopentyl group, a cyclohexyl group, a cycloheptyl group, a cyclooctyl group, an adamantanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a silolanyl group, a phenyl group, a (C₁-C₂₀ alkyl)phenyl group, a biphenyl group, a terphenyl group, a naphthyl group, a 1,2,3,4-tetrahydronaphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, or an azadibenzothiophenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a deuterium-containing C₁-C₂₀ alkyl group, a fluorinated C₁-C₂₀ alkyl group, a C₁-C₂₀ alkoxy group, a cyclopentyl group, a cyclohexyl group, a cycloheptyl group, a cyclooctyl group, an adamantanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.1]heptyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.1]heptyl group, a (C₁-C₂₀ alkyl)bicyclo[2.2.2]octyl group, a silolanyl group, a phenyl group, a (C₁-C₂₀ alkyl)phenyl group, a biphenyl group, a terphenyl group, a naphthyl group, a 1,2,3,4-tetrahydronaphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzoimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, or any combination thereof; or
—N(Q₁)(Q₂), —Si(Q₁)(Q₂)(Q₃), —Ge(Q₁)(Q₂)(Q₃), —C(═O)(Q₁), —S(═O)(Q₁), —S(═O)₂(Q₁), —B(Q₁)(Q₂), —P(Q₁)(Q₂), —P(═O)(Q₁)(Q₂), or —P(═S)(Q₁)(Q₂).

In an embodiment, R₁, R₂, R₁₀, R₂₀, R₃₀, and R₄₀ may each independently be:
a group represented by Formula 2, a group represented by Formula 3, hydrogen, deuterium, —F, —Cl, —Br, —I, —CD₃, —CD₂H, —CDH₂, —CF₃, —CF₂H, —CFH₂, a C₁-C₆₀ alkyl group, a C₂-C₆₀ alkenyl group, a C₂-C₆₀ alkynyl group, or a C₁-C₆₀ alkoxy group; or
a group represented by one of Formulae 9-1 to 9-61, 9-201 to 9-244, 10-1 to 10-154, and 10-201 to 10-350:

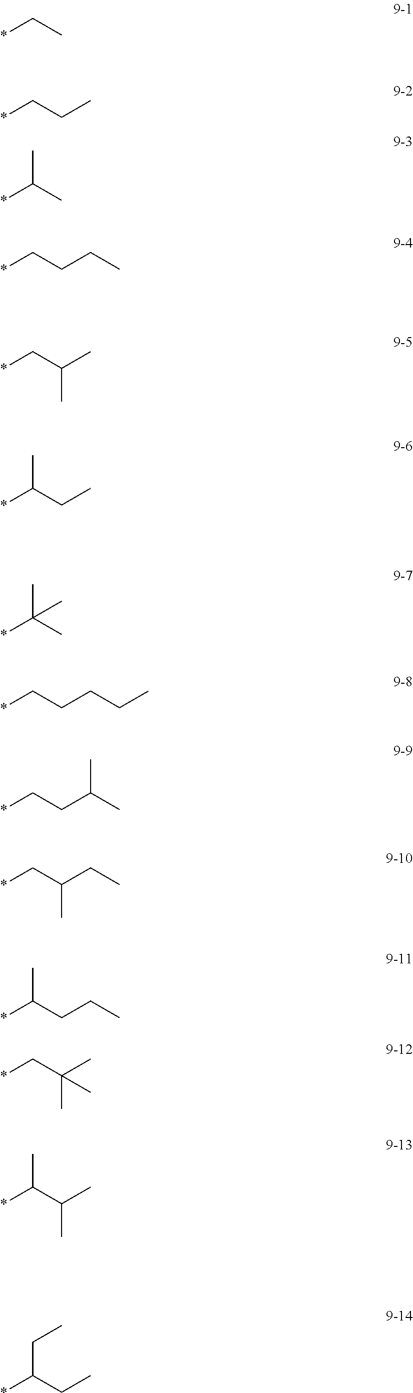

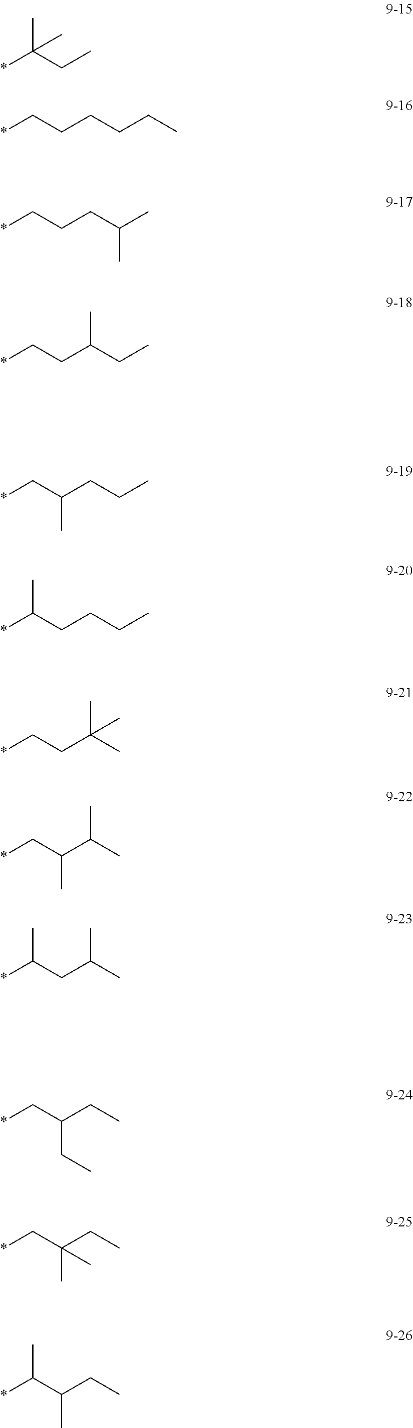

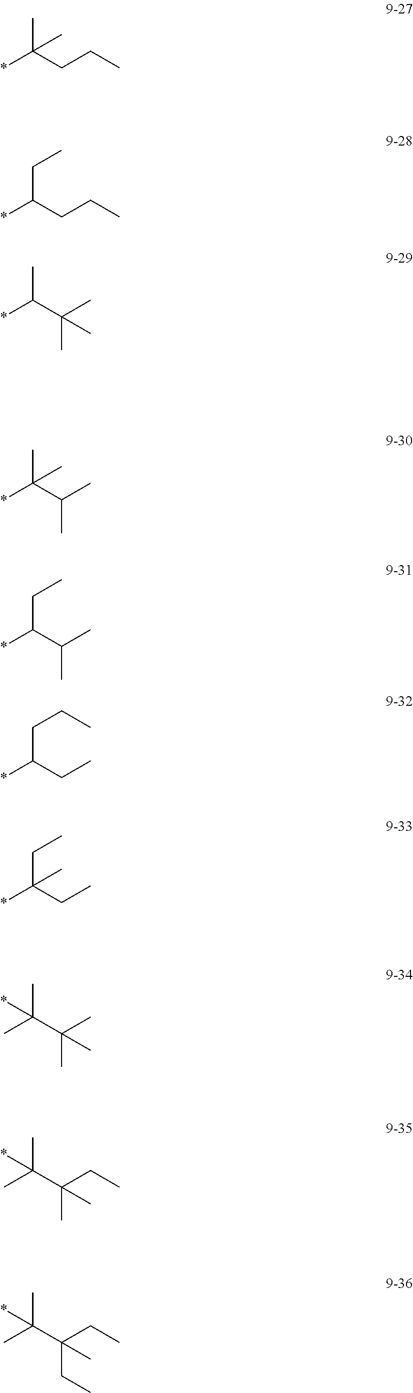

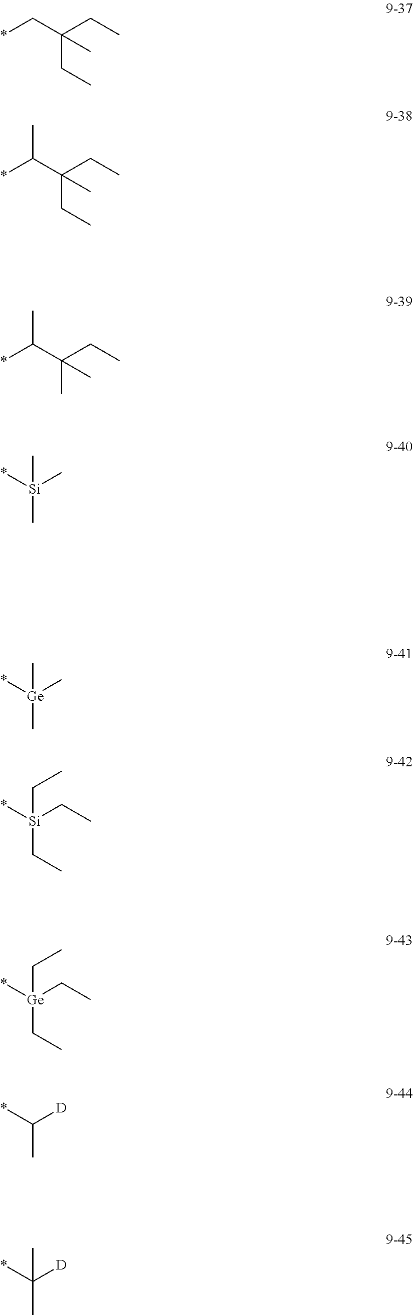

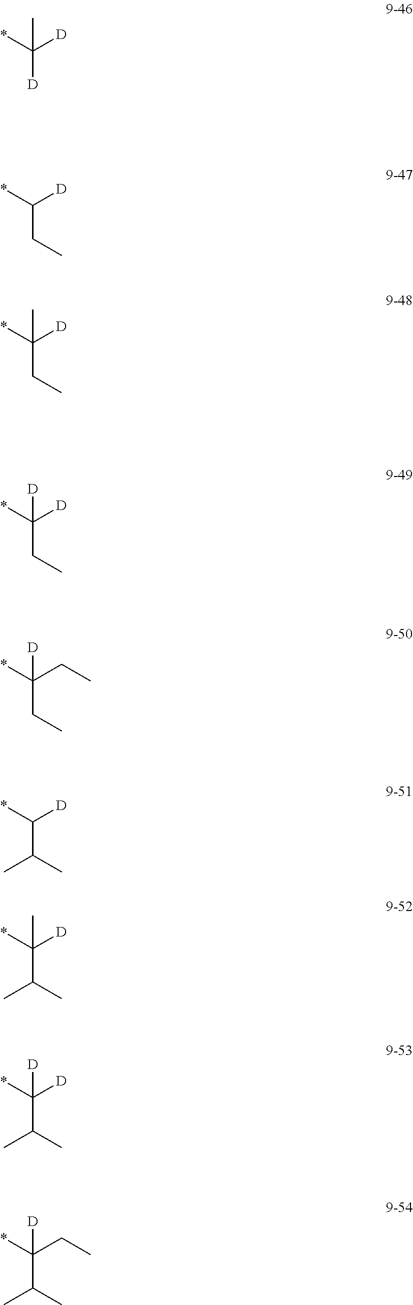

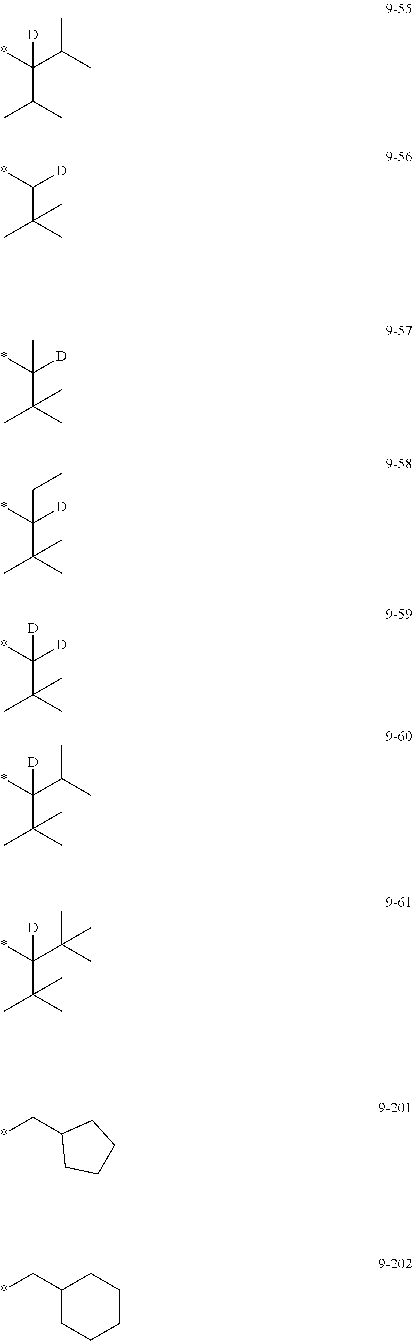

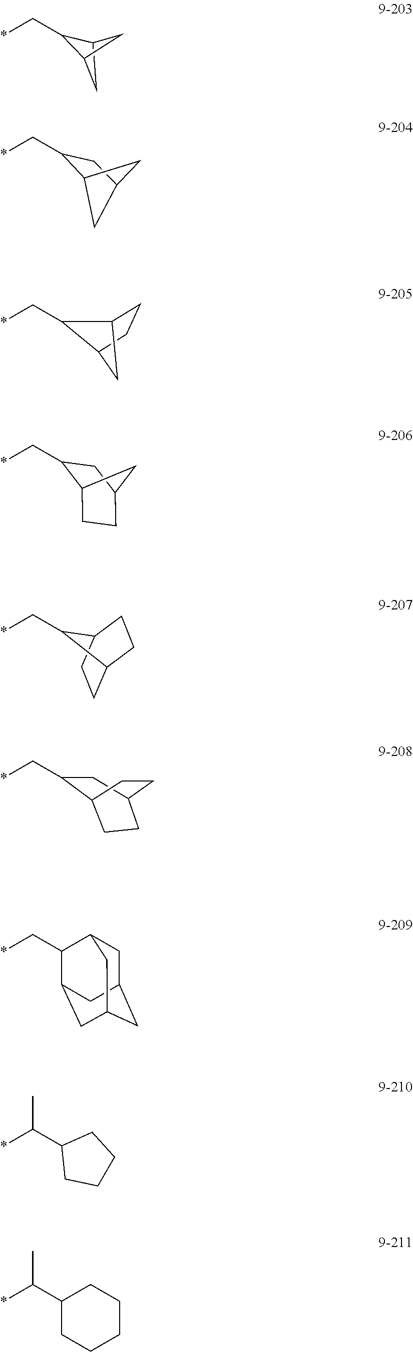

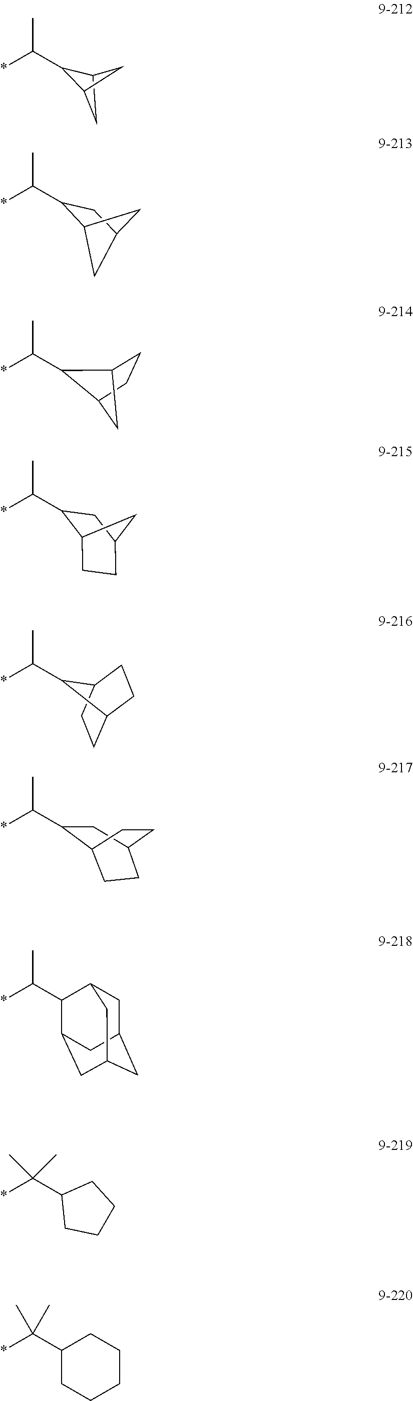

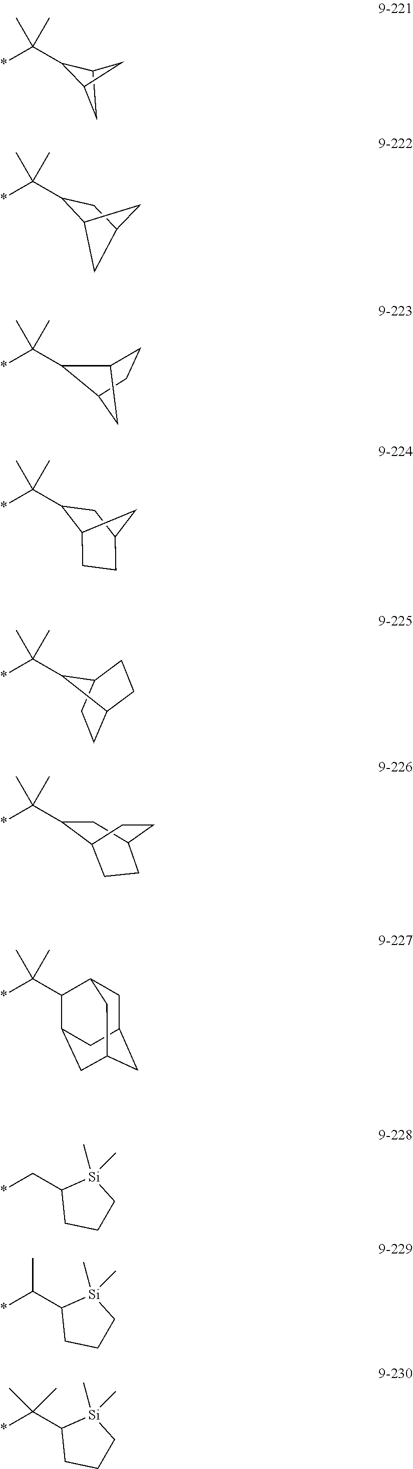

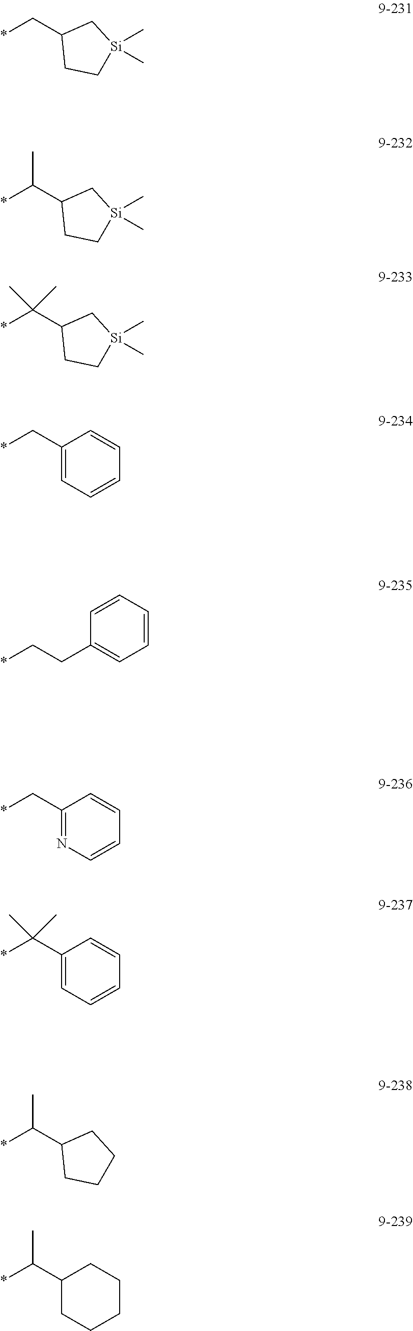

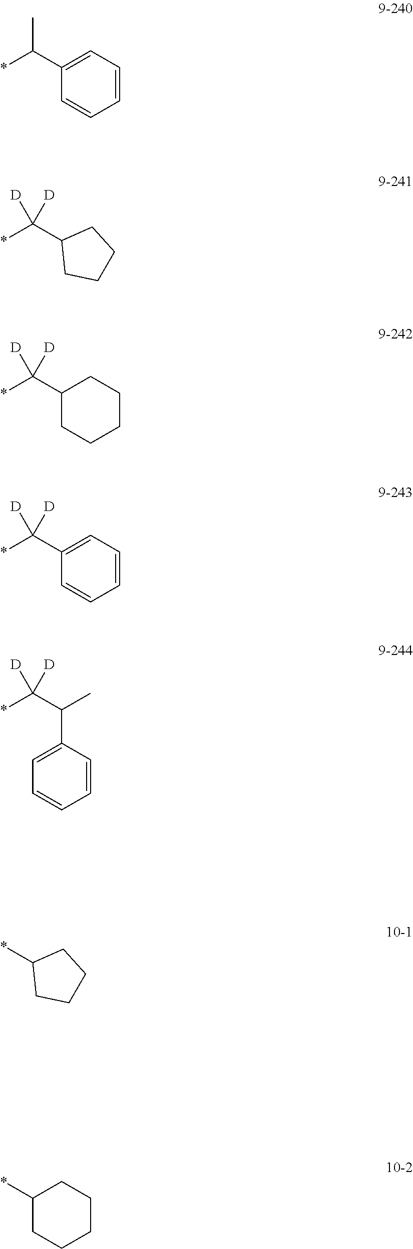

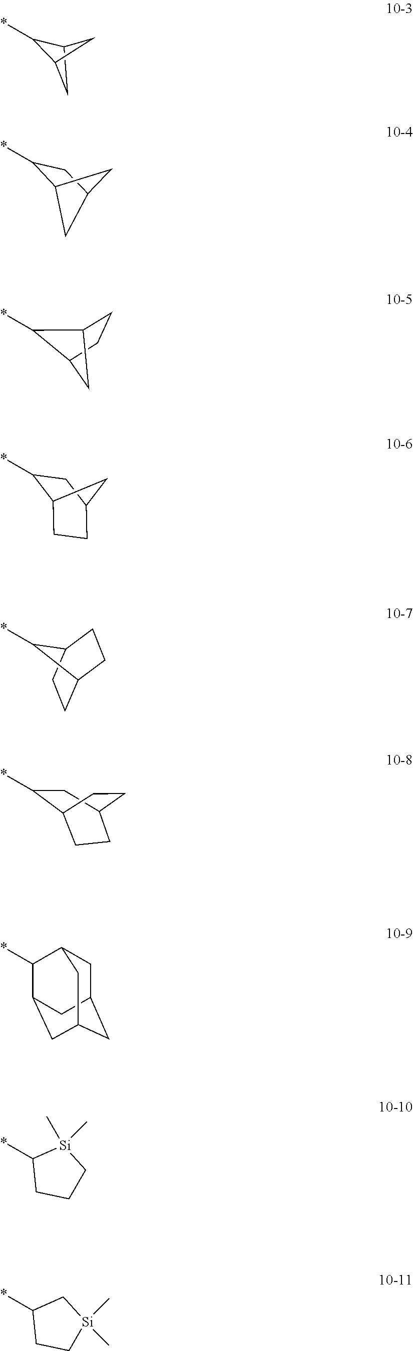

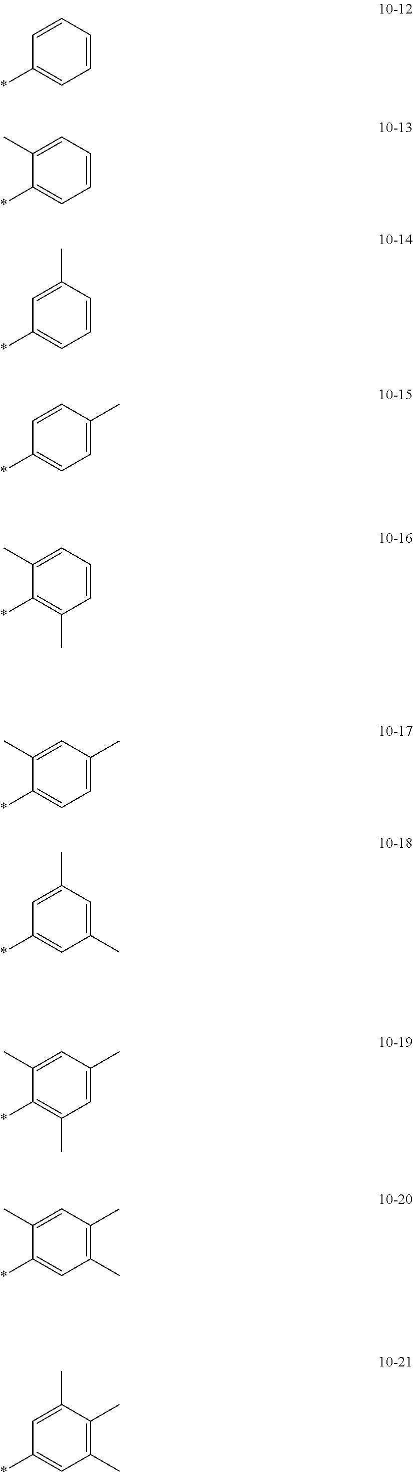

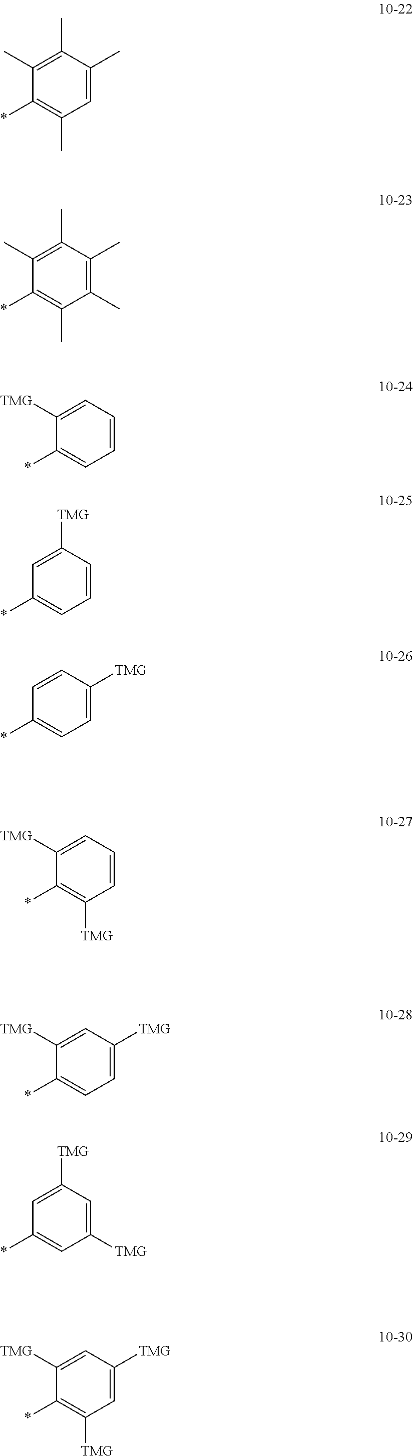

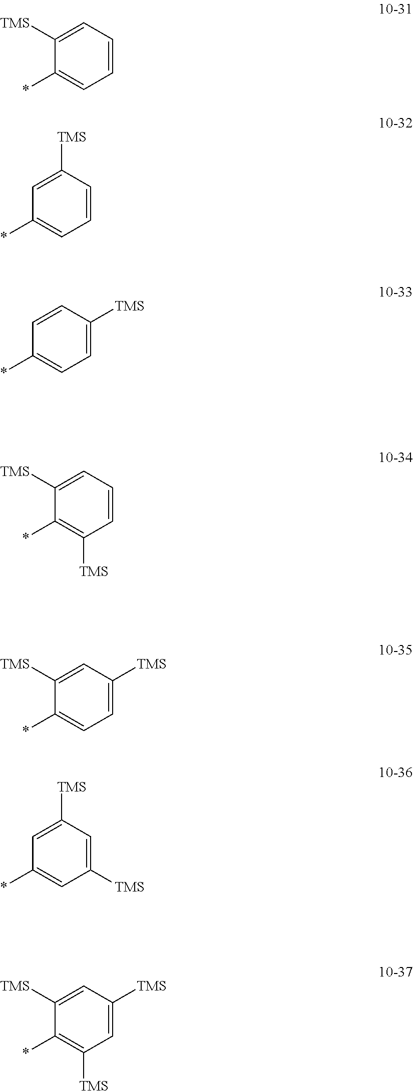

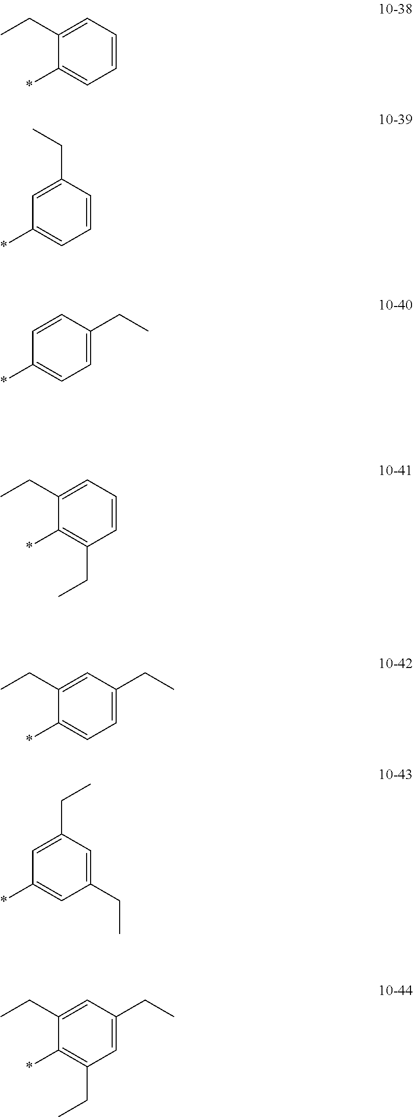

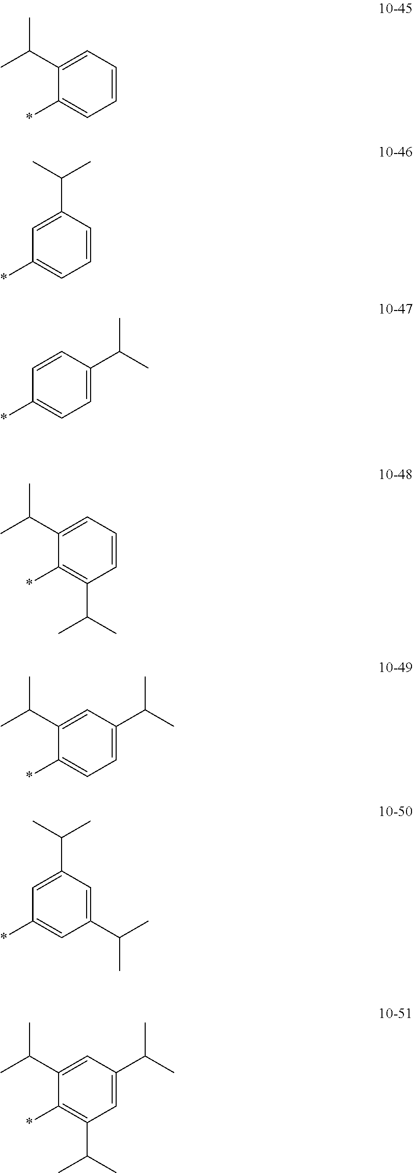

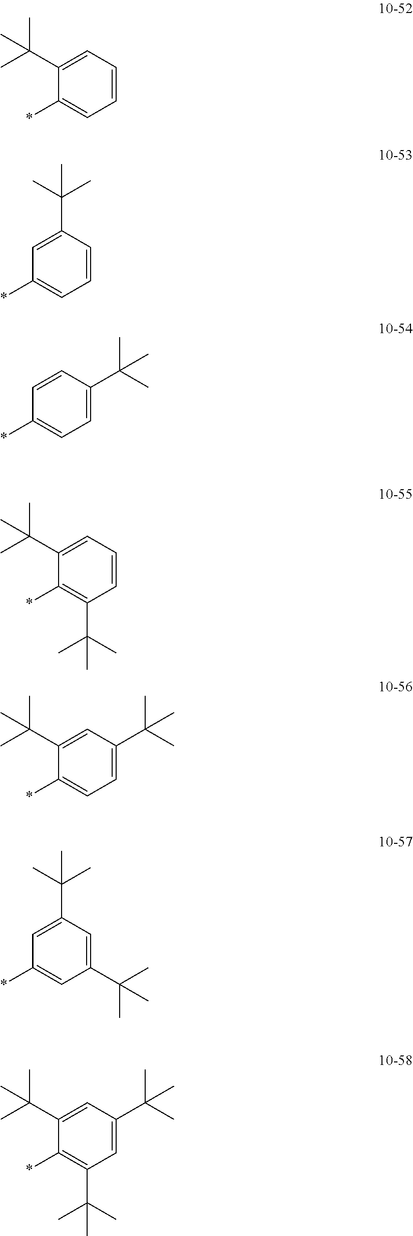

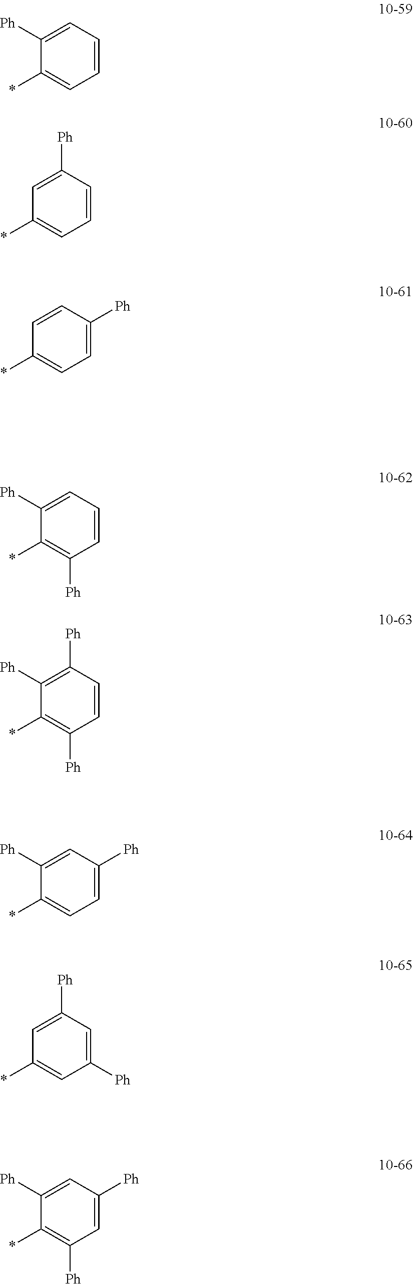

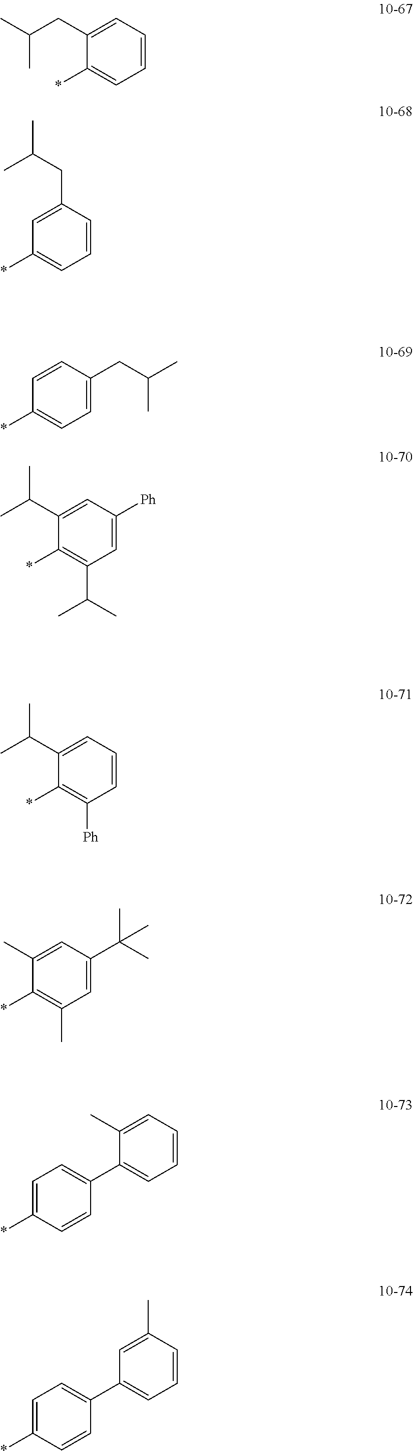

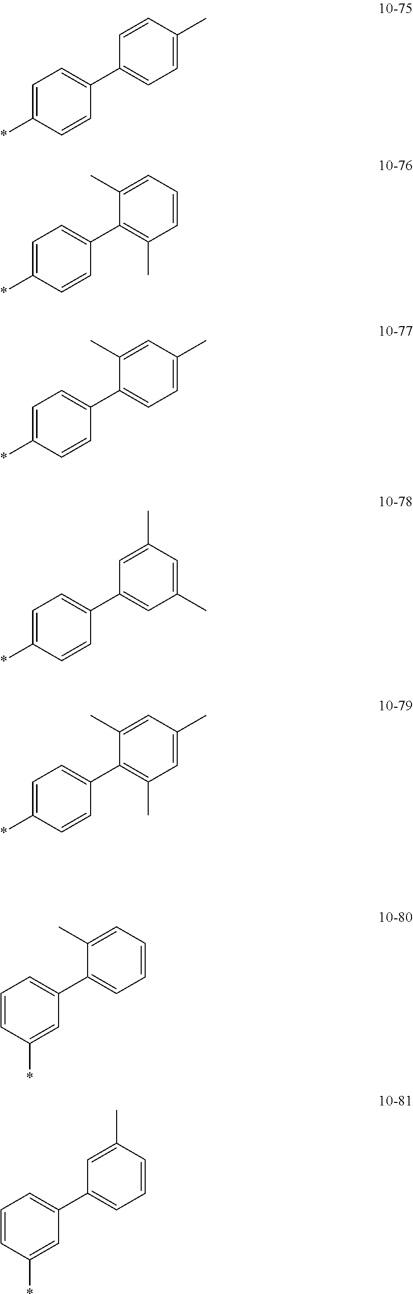

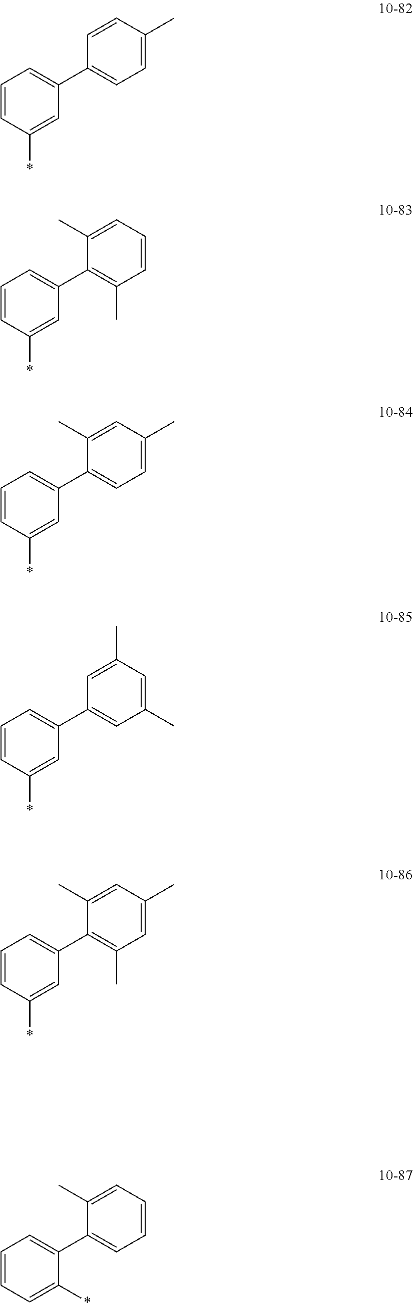

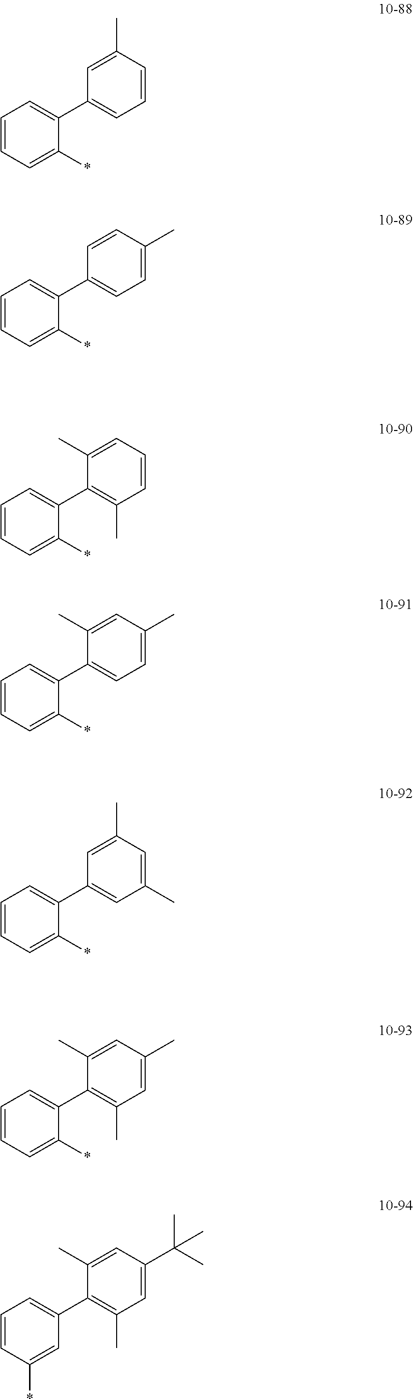

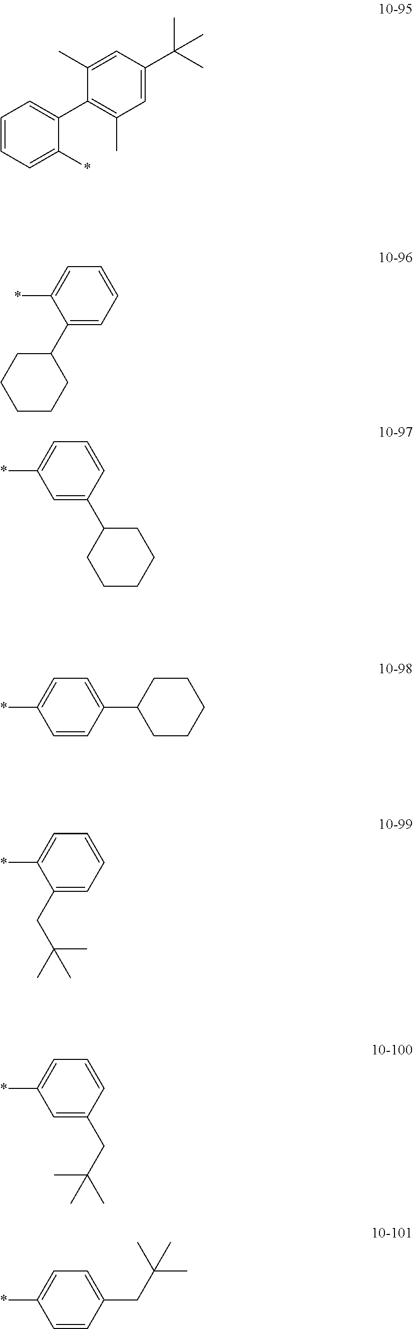

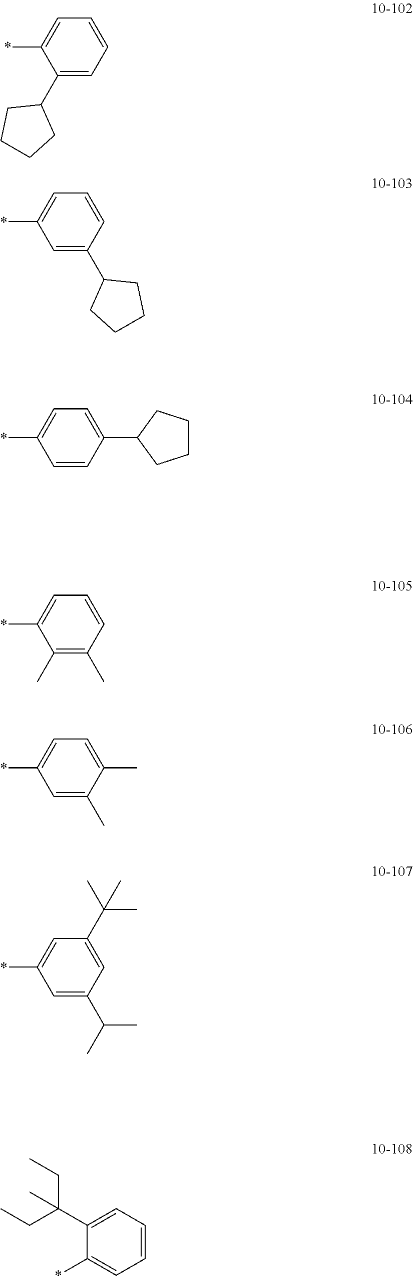

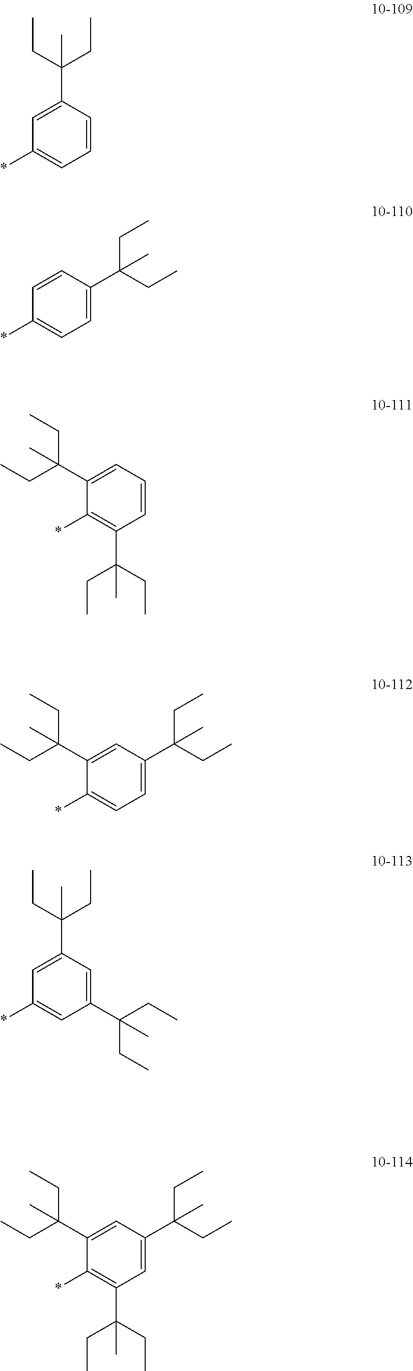

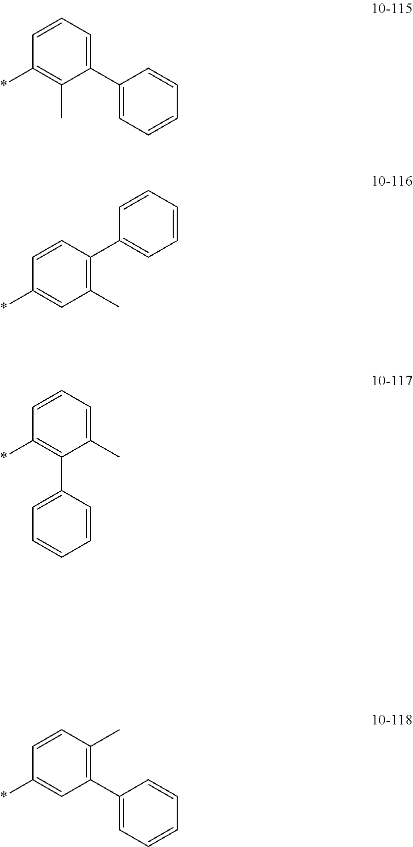

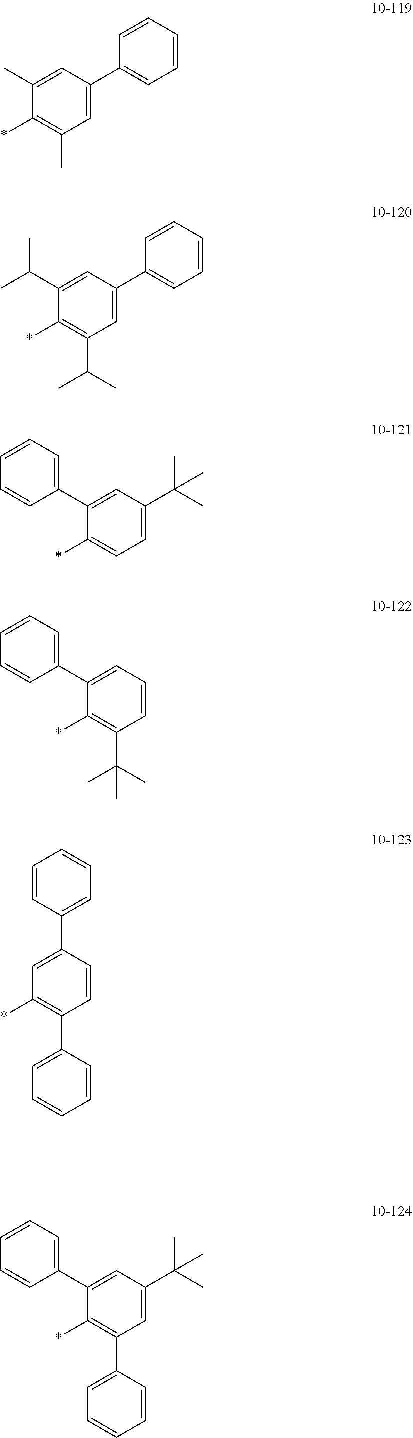

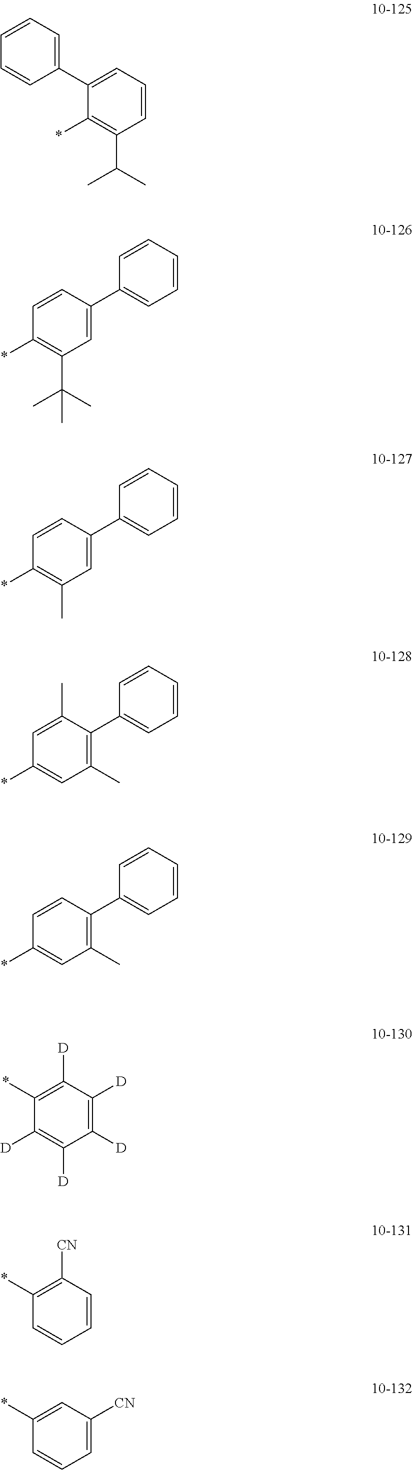

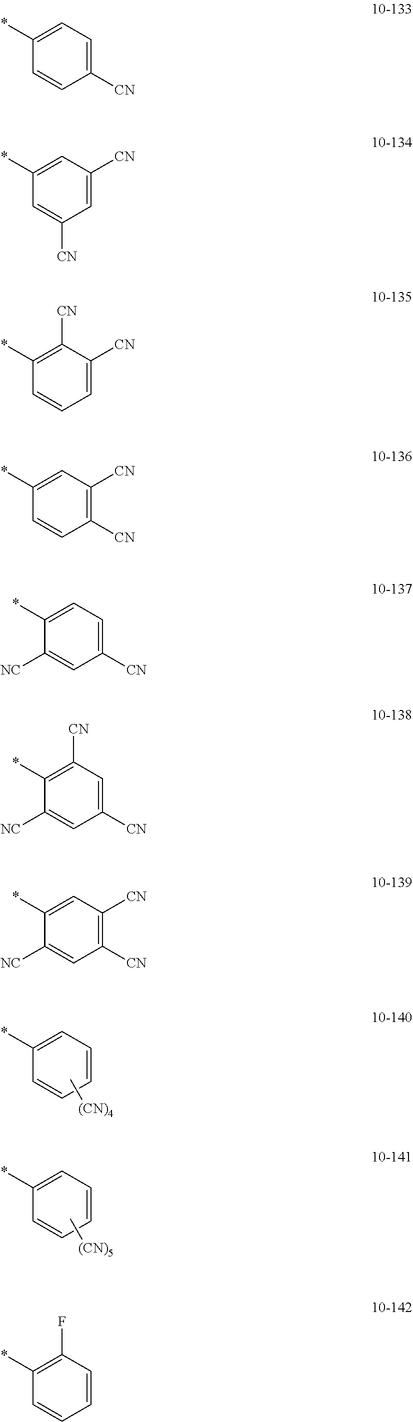

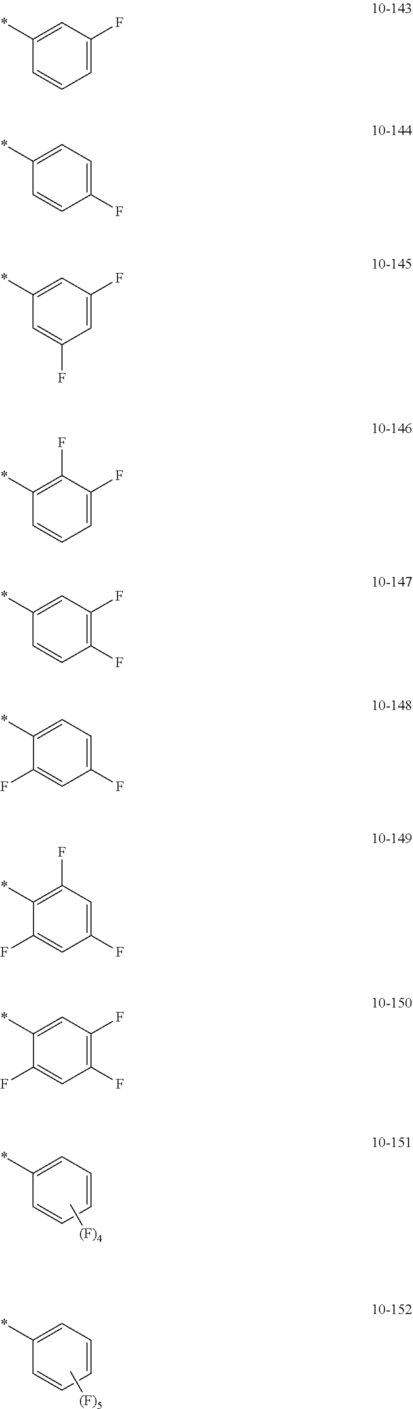

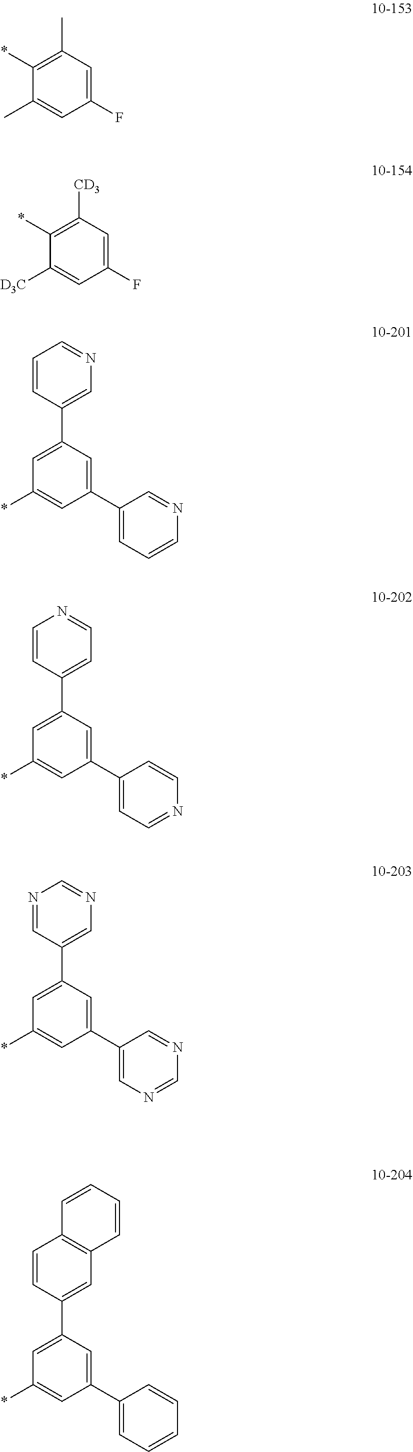

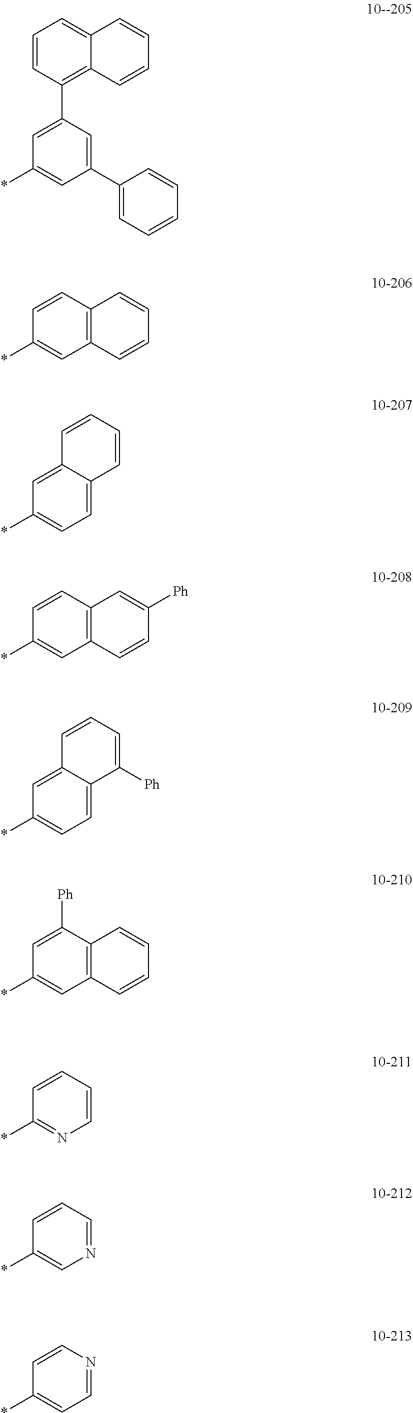

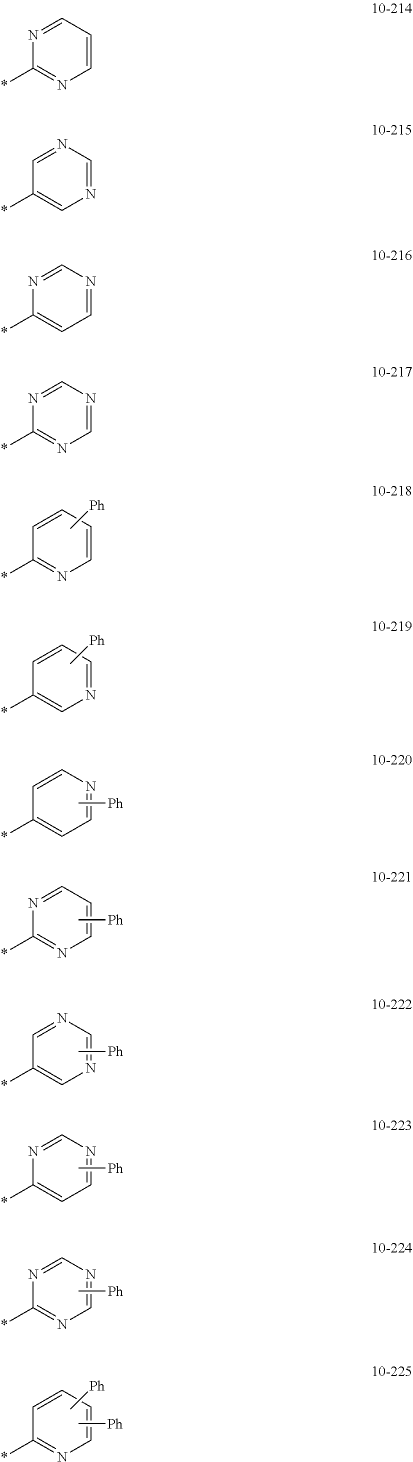

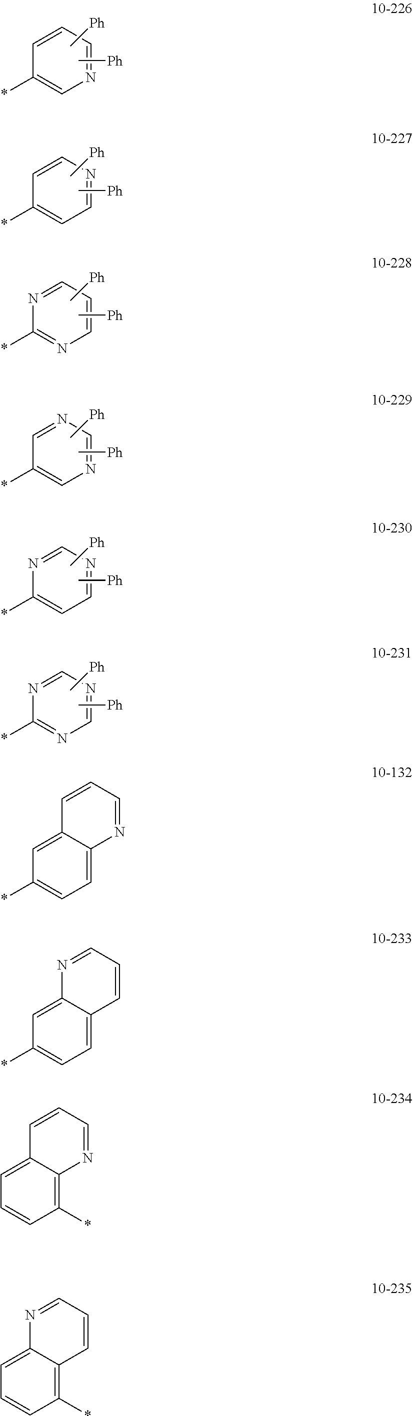

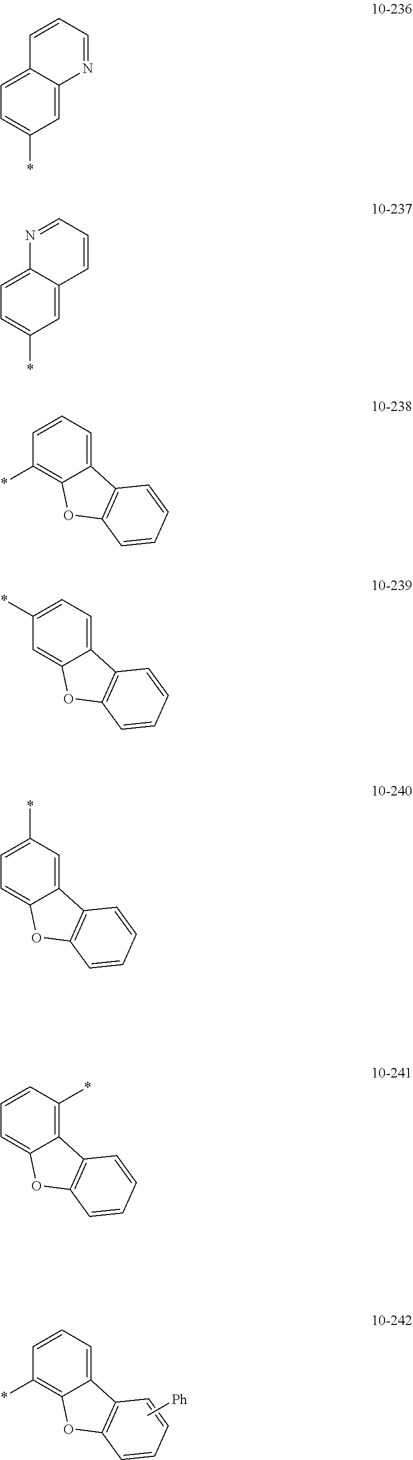

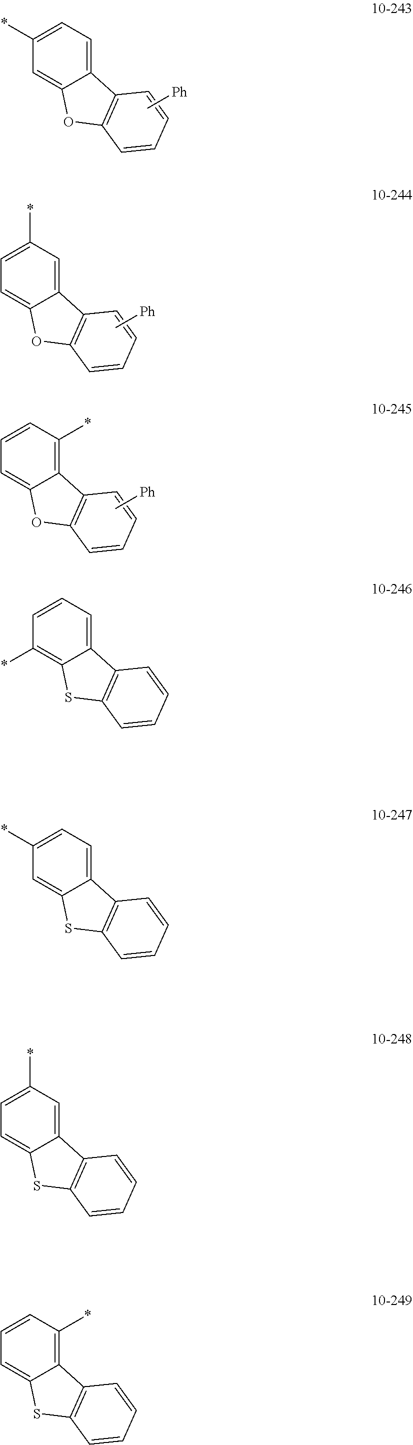

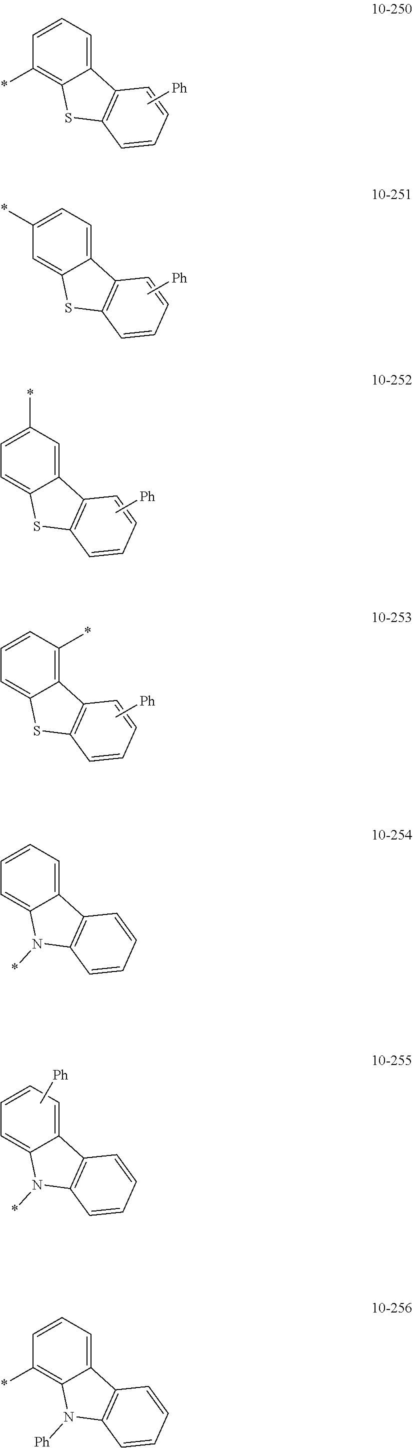

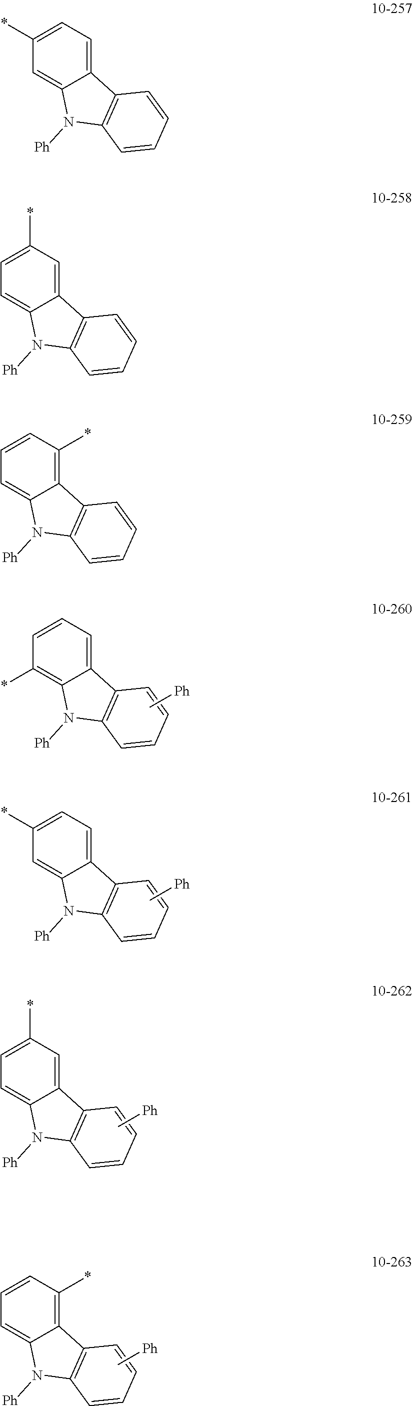

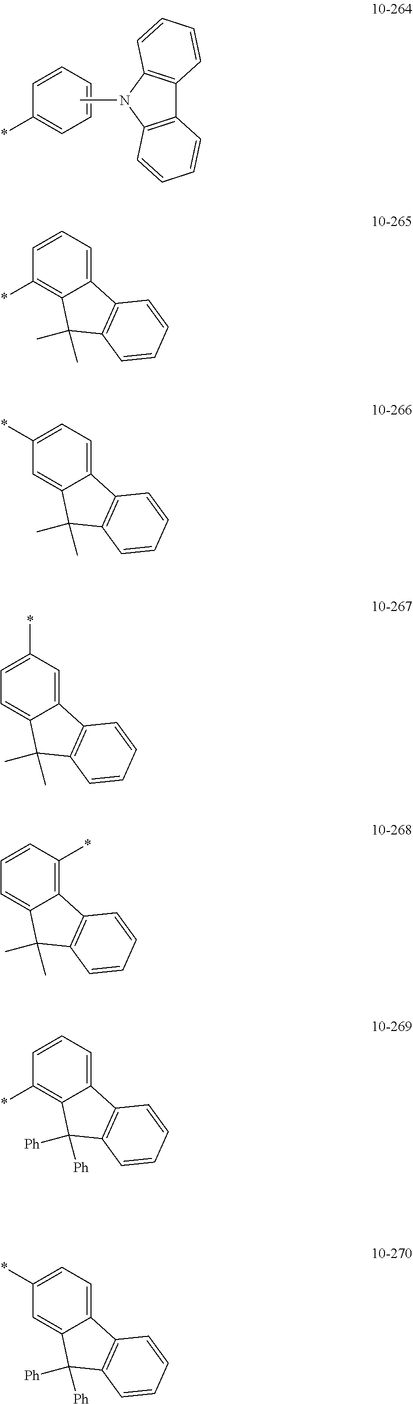

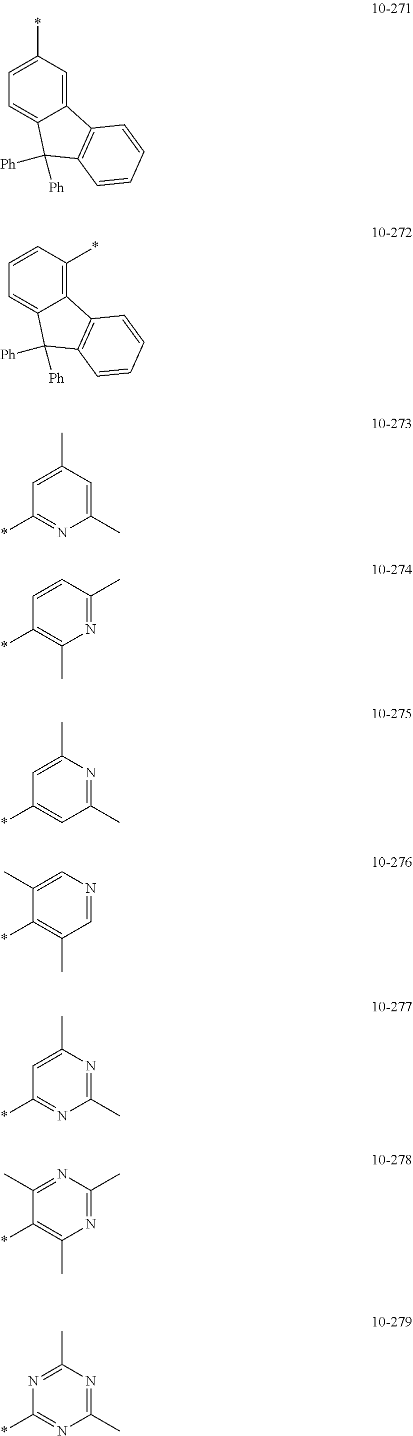

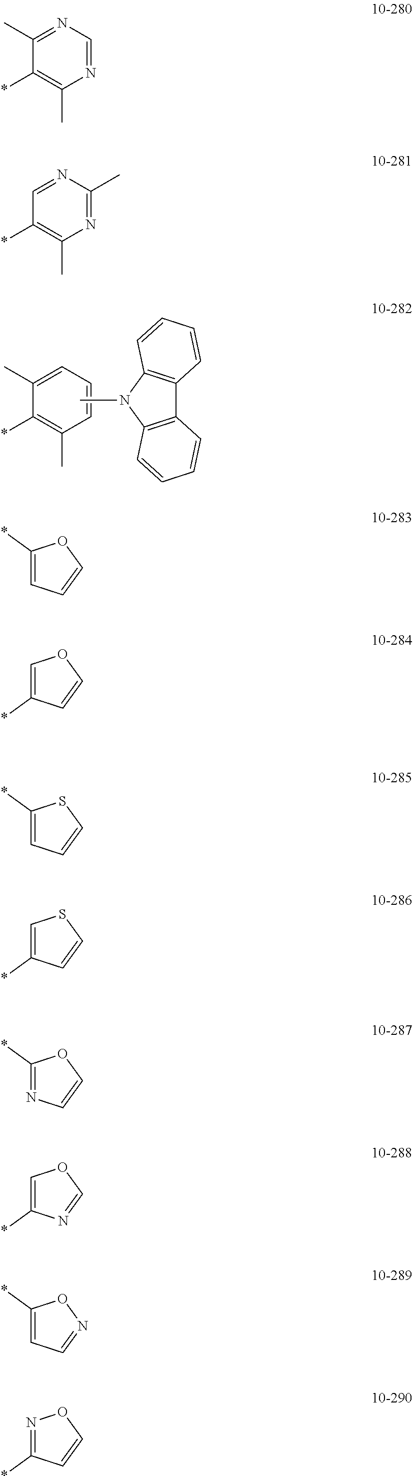

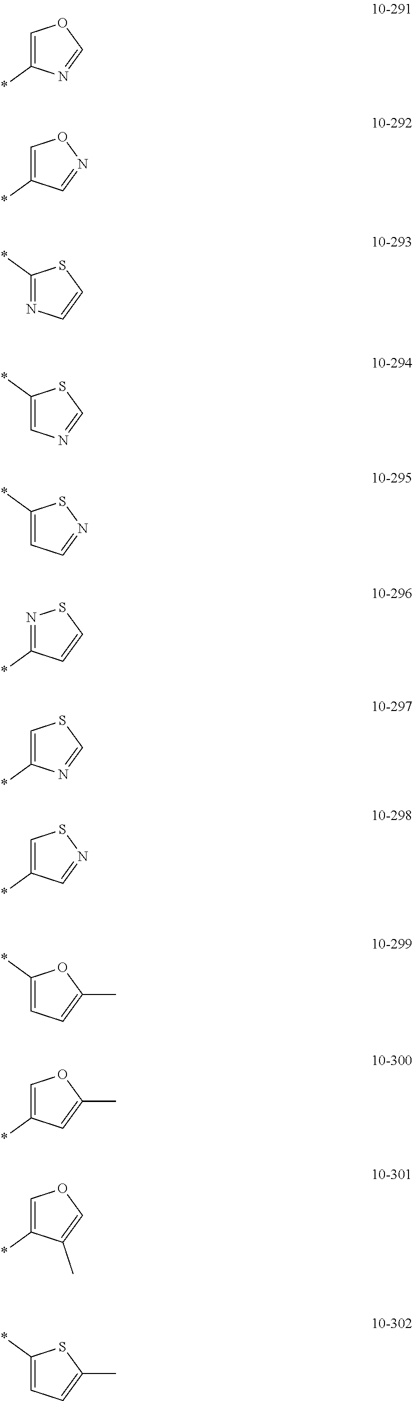

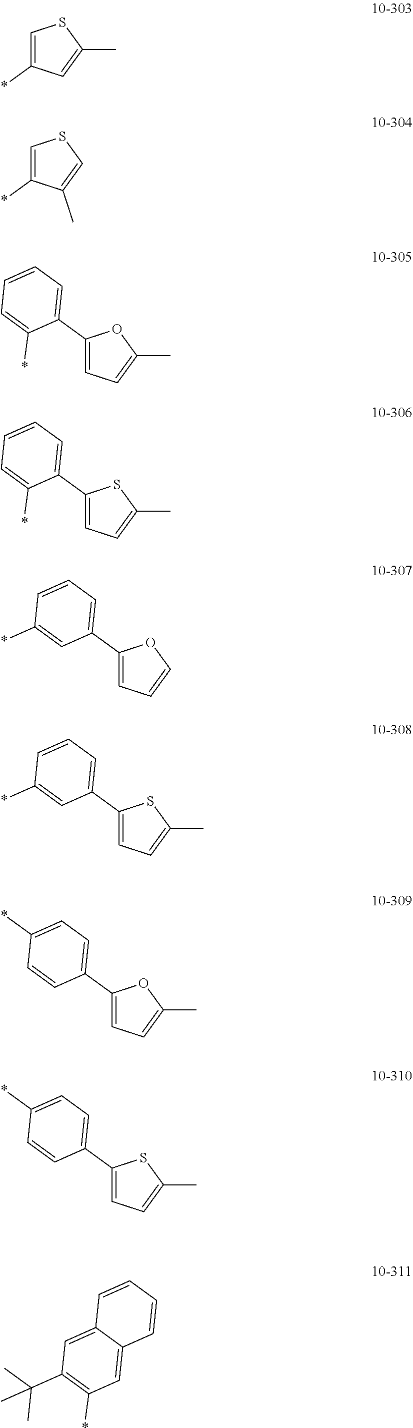

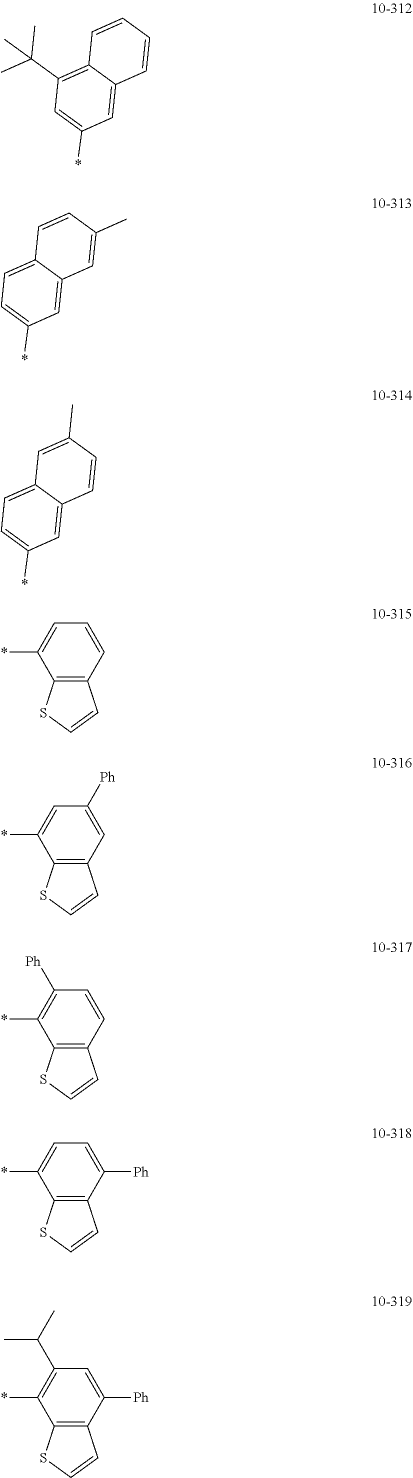

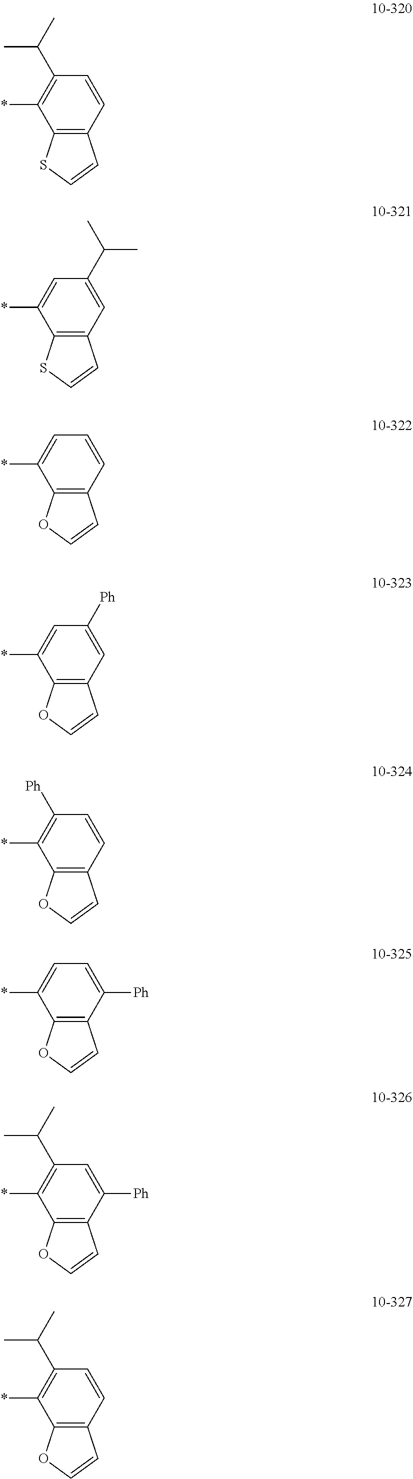

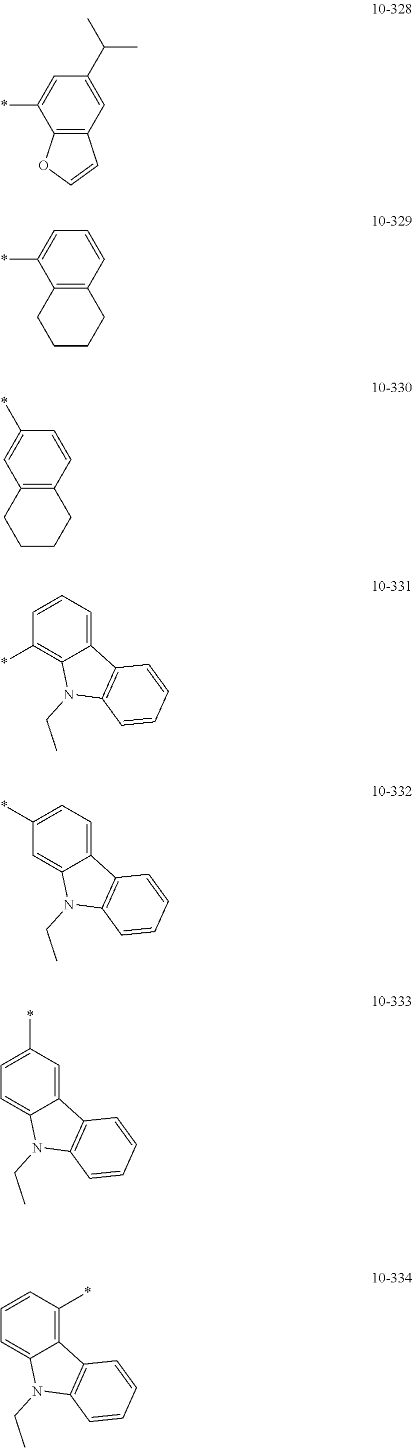

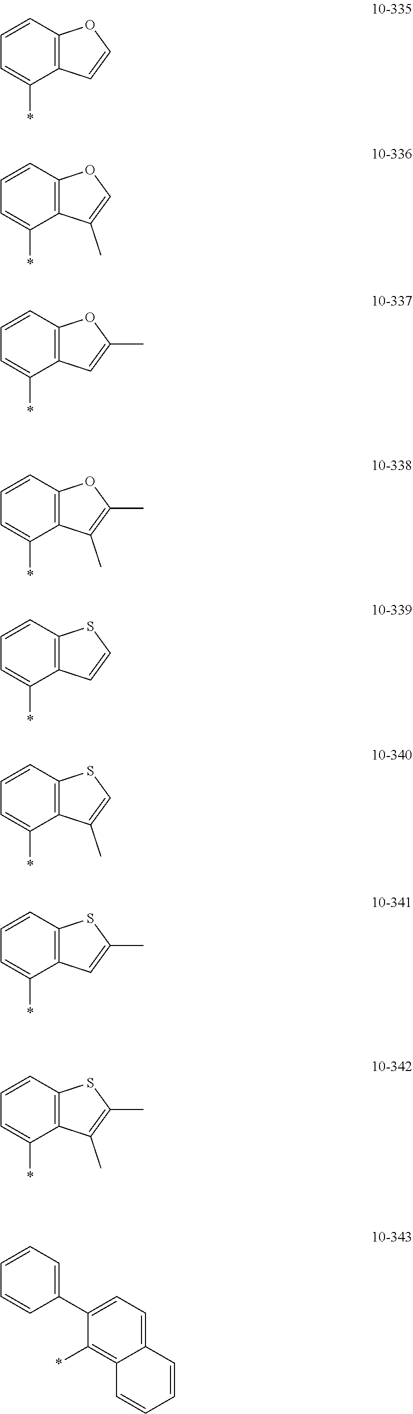

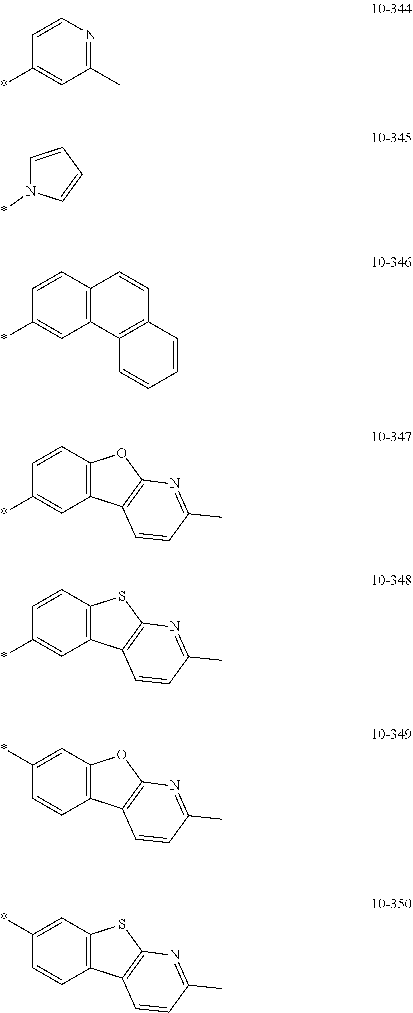

wherein, in Formulae 9-1 to 9-61, 9-201 to 9-244, 10-1 to 10-154, and 10-201 to 10-350* indicates a binding site to a neighboring atom, Ph is a phenyl group, TMS is a trimethylsilyl group, and TMG is a trimethylgermyl group.

In an embodiment, R₅₀ and R₆₀ may each independently be:
a group represented by Formula 3, hydrogen, deuterium, —F, —Cl, —Br, —I, —CD₃, —CD₂H, —CDH₂, —CF₃, —CF₂H, —CFH₂, a C₁-C₆₀ alkyl group, a C₂-C₆₀ alkenyl group, a C₂-C₆₀ alkynyl group, or a C₁-C₆₀ alkoxy group, or
a group represented by one of Formulae 9-1 to 9-61, 9-201 to 9-244, 10-1 to 10-154, and 10-201 to 10-350.

In an embodiment, at least one instance of R₁₀ may be a group represented by Formula 3.

In an embodiment, at least one instance of R₂₀ may be a group represented by Formula 3.

In an embodiment, at least one instance of R₃₀ may be a group represented by Formula 3.

In an embodiment, at least one instance of R₁₀, at least one instance of R₂₀, and at least one instance of R₃₀ may be a group represented by Formula 3. In an embodiment, one of R₃₀ may be a group represented by Formula 3.

In Formulae 1 and 2, b10, b20, b30, b40, and b60 may each independently be 1, 2, 3, 4, 5, 6, 7, or 8.

In Formula 2, b50 may be 1, 2, or 3.

In an embodiment, a group represented by Formula 3 may be a group represented by one of Formulae 3-1 to 3-3:

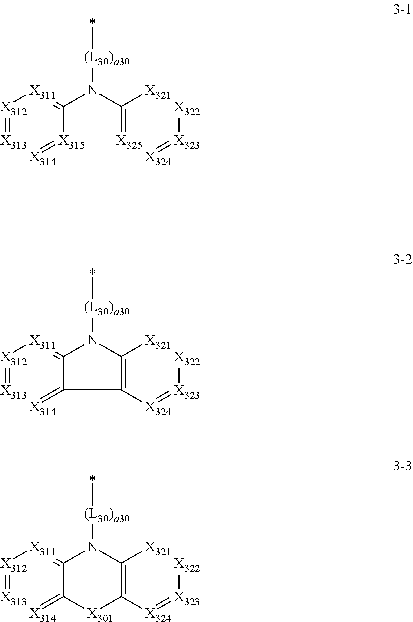

wherein, in Formulae 3-1 to 3-3,
L₃₀ and a30 may each be the same as described in the present specification,
X₃₀₁ may be the same as described in connection with X₃₁,
X₃₁₁ may be C(R₃₁₁) or N, X₃₁₂ may be C(R₃₁₂) or N, X₃₁₃ may be C(R₃₁₃) or N, X₃₁₄ may be C(R₃₁₄) or N,
X₃₂₁ may be C(R₃₂₁) or N, X₃₂₂ may be C(R₃₂₂) or N, X₃₂₃ may be C(R₃₂₃) or N, X₃₂₄ may be C(R₃₂₄) or N,
R₃₁₁ to R₃₁₄ may each independently be the same as described in connection with R₃₁₀,
R₃₂₁ to R₃₂₄ may each independently be the same as described in connection with R₃₂₀, and
* may indicate a binding site to a neighboring atom.

In an embodiment, the heterocyclic compound may be represented by Formula 1A or 1B:

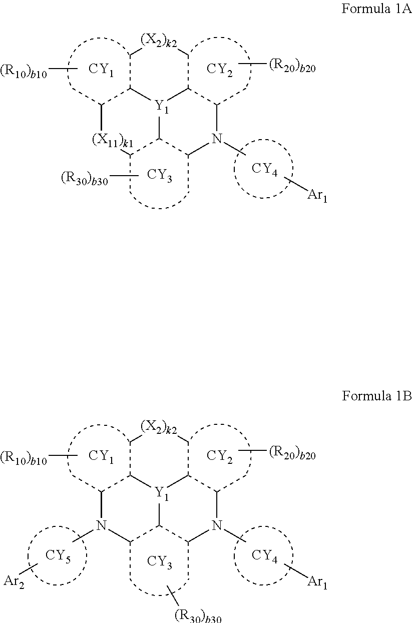

wherein, in Formulae 1A and 1B,
Y₁, X₂, k1, k2, CY₁ to CY₅, CY₁₁, R₁, R₂, R₁₀, R₂₀, R₃₀, b10, b20, and b30 may each be the same as described in the present specification, and
X₁₁ may be a single bond, O, S, Se, N(R₁), C(R₁)(R₂), Si(R₁)(R₂), Ge(R₁)(R₂), B(R₁), P(R₁), P(═O)(R₁), S(═O)₂, or C(═O).

In an embodiment, the heterocyclic compound may be represented by one of Formulae 11-1 to 11-9:

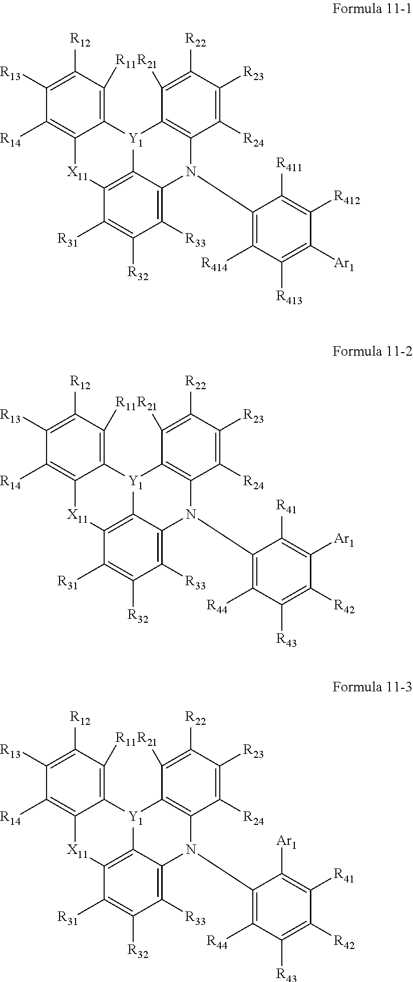

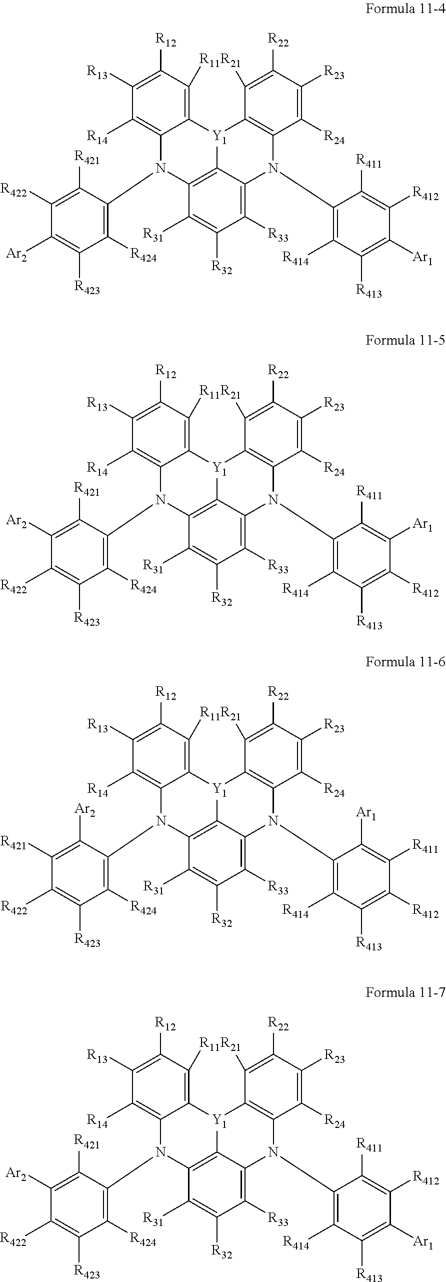

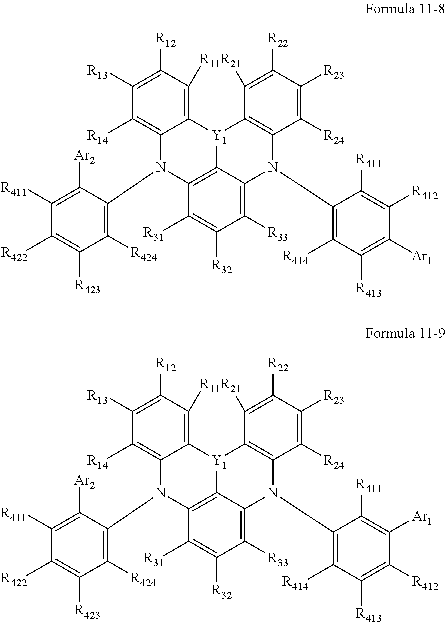

wherein, in Formulae 11-1 to 11-9,
Y₁ and X₁ may each be the same as described in the present specification,
X₁₁ may be a single bond, O, S, Se, N(R₁), C(R₁)(R₂), Si(R₁)(R₂), Ge(R₁)(R₂), B(R₁), P(R₁), P(═O)(R₁), S(═O)₂, or C(═O),
R₁₁ to R₁₄ may each independently be the same as described in connection with R₁₀,
R₂₁ to R₂₄ may each independently be the same as described in connection with R₂₀,
R₃₁ to R₃₃ may each independently be the same as described in connection with R₃₀, and
R₄₁₁ to R₄₁₄ and R₄₂₁ to R₄₂₄ may each independently be the same as described in connection with R₄₀.

In an embodiment, at least one of R₁₁ to R₁₄ may be a group represented by Formula 3.

In an embodiment, at least one of R₂₁ to R₂₄ may be a group represented by Formula 3.

In an embodiment, at least one of R₃₁ to R₃₃ may be a group represented by Formula 3.

In an embodiment, at least one of R₁₁ to R₁₄, at least one of R₂₁ to R₂₄, and at least one of R₃₁ to R₃₃ may be a group represented by Formula 3.

In an embodiment, the heterocyclic compound may have a symmetric structure or an asymmetric structure.

For example, the heterocyclic compound may have a symmetric structure.

As another example, the heterocyclic compound may have an asymmetric structure.

As used herein, “a symmetric structure” means that when is drawn down the middle of the structure (i.e., through the B and center of the CY₃ ring Formula I), each side is the same. For example, Compound 1 has a symmetric structure, whereas Compound 25 has an asymmetric structure.

A substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₁)(Q₁₂)(Q₁₃), —Ge(Q₁₁)(Q₁₂)(Q₁₃), —C(═O)(Q₁₁), —S(═O)(Q₁₁), —S(═O)₂(Q₁₁), —B(Q₁₁)(Q₁₂), —P(Q₁₁)(Q₁₂), —P(═O)(Q₁₁)(Q₁₂), —P(═S)(Q₁₁)(Q₁₂), or any combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₁)(Q₂₂)(Q₂₃), —Ge(Q₂₁)(Q₂₂)(Q₂₃), —C(═O)(Q₂₁), —S(═O)(Q₂₁), —S(═O)₂(Q₂₁), —B(Q₂₁)(Q₂₂), —P(Q₂₁)(Q₂₂), —P(═O)(Q₂₁)(Q₂₂), —P(═S)(Q₂₁)(Q₂₂), or any combination thereof;
—N(Q₃₁)(Q₃₂), —Si(Q₃₁)(Q₃₂)(Q₃₃), —Ge(Q₃₁)(Q₃₂)(Q₃₃), —C(═O)(Q₃₁), —S(═O)(Q₃₁), —S(═O)₂(Q₃₁), —B(Q₃₁)(Q₃₂), —P(Q₃₁)(Q₃₂), —P(═O)(Q₃₁)(Q₃₂), or —P(Q₃₁)(Q₃₂).

Q₁ to Q₃, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; an amidino group; a hydrazine group; a hydrazone group; a carboxylic acid or a salt thereof; a sulfonic acid or a salt thereof; a phosphoric acid or a salt thereof; a C₁-C₆₀ alkyl group that is unsubstituted or substituted with deuterium, a C₁-C₆₀ alkyl group, a C₆-C₆₀ aryl group, or any combination thereof; a C₂-C₆₀ alkenyl group; a C₂-C₆₀ alkynyl group; a C₁-C₆₀ alkoxy group; a C₃-C₁₀ cycloalkyl group; a C₁-C₁₀ heterocycloalkyl group; a C₃-C₁₀ cycloalkenyl group; a C₁-C₁₀ heterocycloalkenyl group; a C₆-C₆₀ aryl group that is unsubstituted or substituted with deuterium, a C₁-C₆₀ alkyl group, a C₆-C₆₀ aryl group, or any combination thereof; a C₆-C₆₀ aryloxy group; a C₆-C₆₀ arylthio group; a C₁-C₆₀ heteroaryl group; a monovalent non-aromatic condensed polycyclic group; or a monovalent non-aromatic condensed heteropolycyclic group.

For example, Q₁ to Q₃, Q₁₁ to Q₁₃, Q₂₁ to Q₂₃, and Q₃₁ to Q₃₃ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or
an n-propyl group, an iso-propyl group, an n-butyl group, a sec-butyl group, an isobutyl group, a tert-butyl group, an n-pentyl group, a tert-pentyl group, a neo-pentyl group, an isopentyl group, a sec-pentyl group, a 3-pentyl group, a sec-isopentyl group, a phenyl group, a biphenyl group, or a naphthyl group, each unsubstituted or substituted with deuterium, a C₁-C₁₀ alkyl group, a phenyl group, or any combination thereof.

In an embodiment, the heterocyclic compound may be at least one of Compounds 1 to 792, but embodiments are not limited thereto:

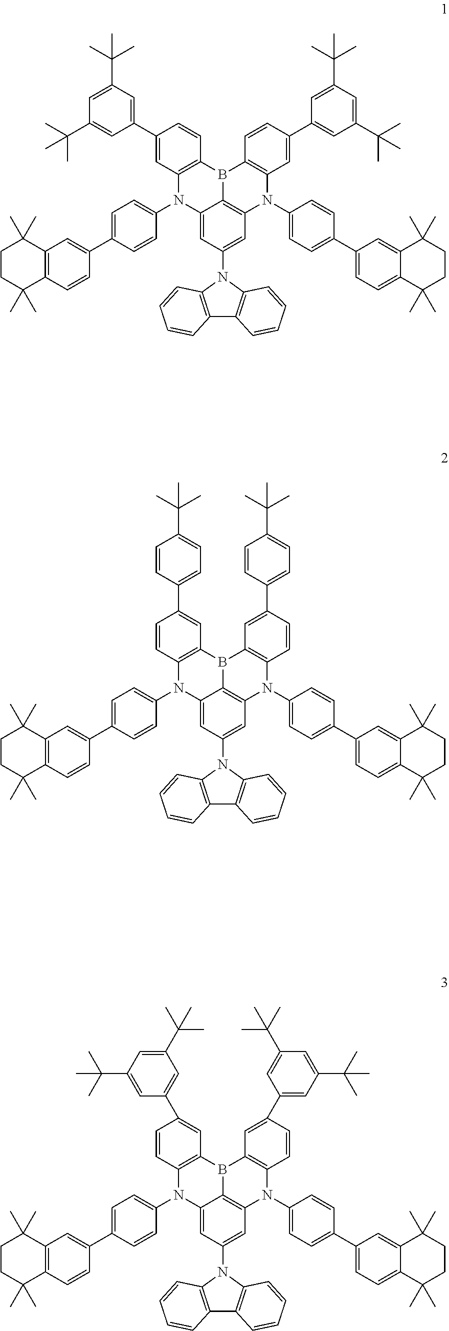

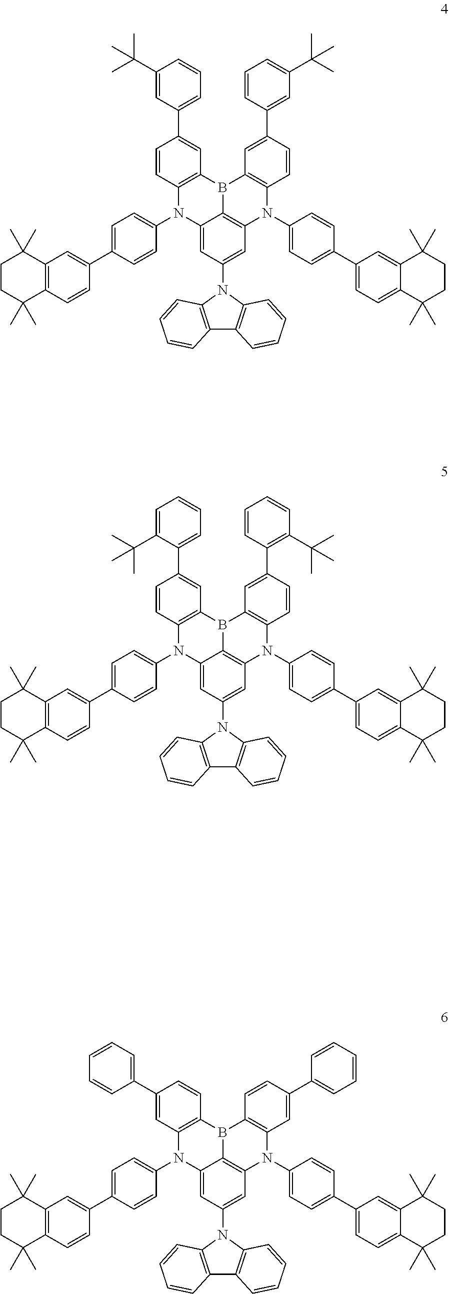 [SMILES: CC(C)(C)c1cccc(-c2ccc3c(c2)B2c4cc(-c5cccc(C(C)(C)C)c5)ccc4N(c4ccc(-c5ccc6c(c5)C(C)(C)CCC6(C)C)cc4)c4cc(-n5c6ccccc6c6ccccc65)cc(c42)N3c2ccc(-c3ccc4c(c3)C(C)(C)CCC4(C)C)cc2)c1 ; CC(C)(C)c1ccccc1-c1ccc2c(c1)B1c3cc(-c4ccccc4C(C)(C)C)ccc3N(c3ccc(-c4ccc5c(c4)C(C)(C)CCC5(C)C)cc3)c3cc(-n4c5ccccc5c5ccccc54)cc(c31)N2c1ccc(-c2ccc3c(c2)C(C)(C)CCC3(C)C)cc1 ; CC1(C)CCC(C)(C)c2cc(-c3ccc(N4c5cc(-c6ccccc6)ccc5B5c6ccc(-c7ccccc7)cc6N(c6ccc(-c7ccc8c(c7)C(C)(C)CCC8(C)C)cc6)c6cc(-n7c8ccccc8c8ccccc87)cc4c65)cc3)ccc21]

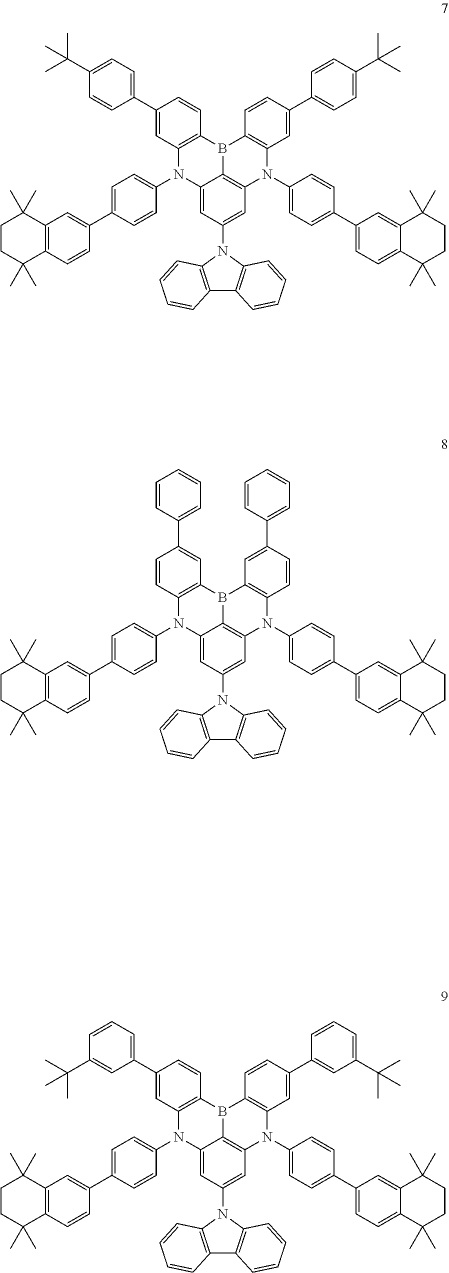 [SMILES: CC(C)(C)c1ccc(-c2ccc3c(c2)N(c2ccc(-c4ccc5c(c4)C(C)(C)CCC5(C)C)cc2)c2cc(-n4c5ccccc5c5ccccc54)cc4c2B3c2ccc(-c3ccc(C(C)(C)C)cc3)cc2N4c2ccc(-c3ccc4c(c3)C(C)(C)CCC4(C)C)cc2)cc1 ; CC1(C)CCC(C)(C)c2cc(-c3ccc(N4c5ccc(-c6ccccc6)cc5B5c6cc(-c7ccccc7)ccc6N(c6ccc(-c7ccc8c(c7)C(C)(C)CCC8(C)C)cc6)c6cc(-n7c8ccccc8c8ccccc87)cc4c65)cc3)ccc21 ; CC(C)(C)c1cccc(-c2ccc3c(c2)N(c2ccc(-c4ccc5c(c4)C(C)(C)CCC5(C)C)cc2)c2cc(-n4c5ccccc5c5ccccc54)cc4c2B3c2ccc(-c3cccc(C(C)(C)C)c3)cc2N4c2ccc(-c3ccc4c(c3)C(C)(C)CCC4(C)C)cc2)c1]

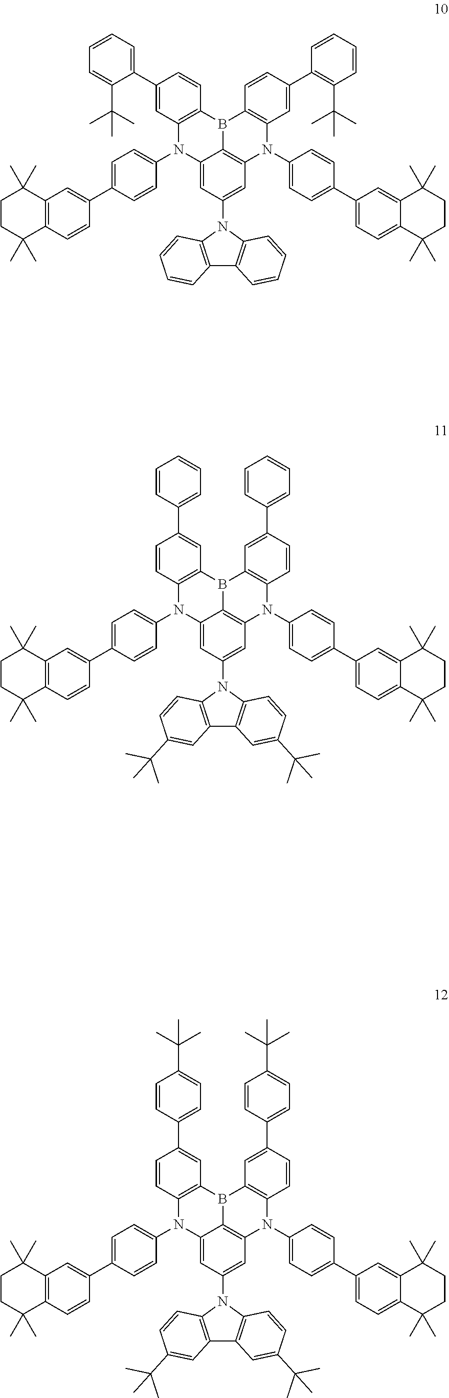

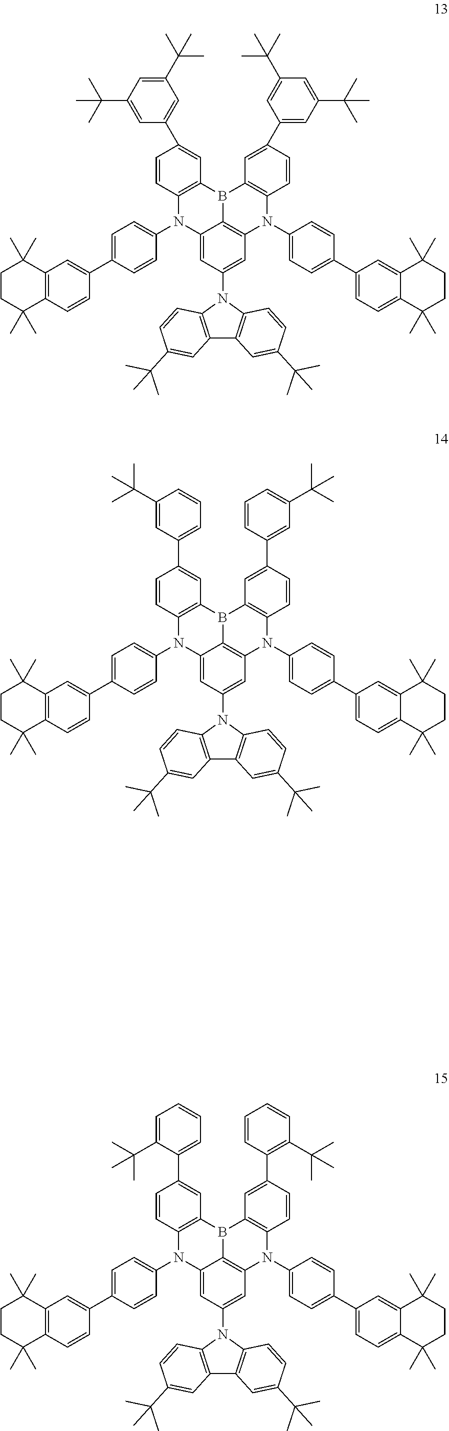

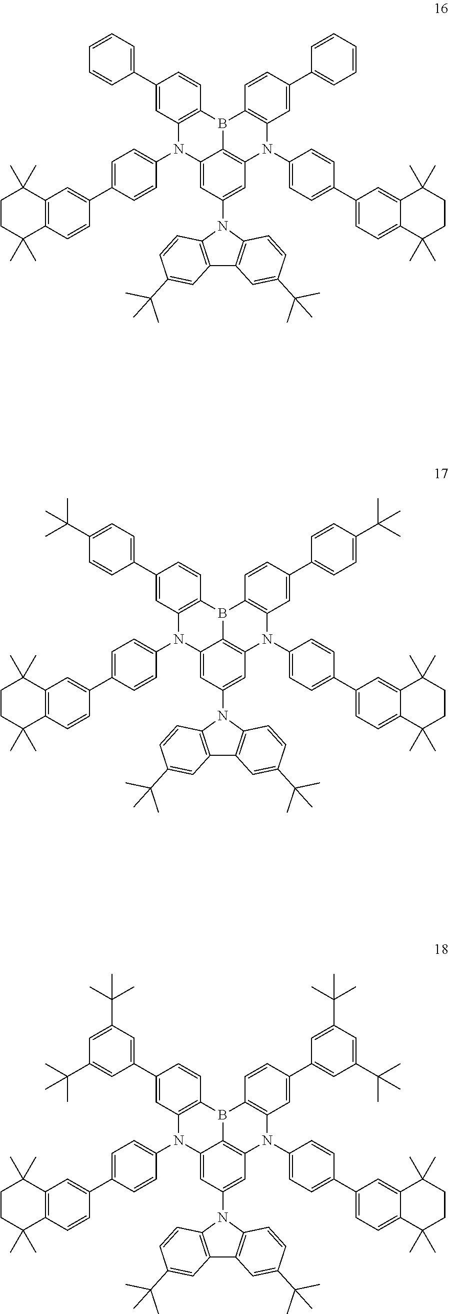

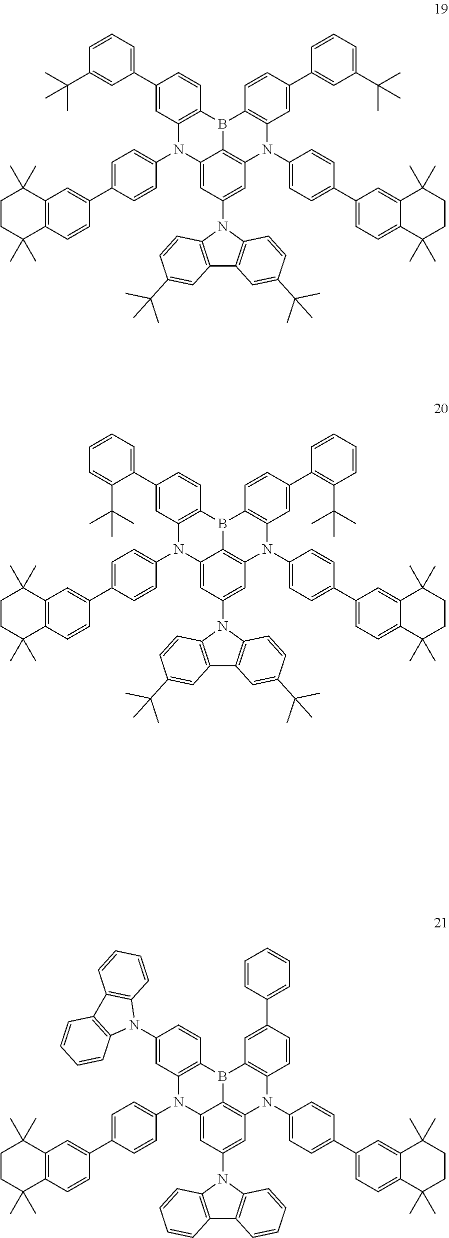

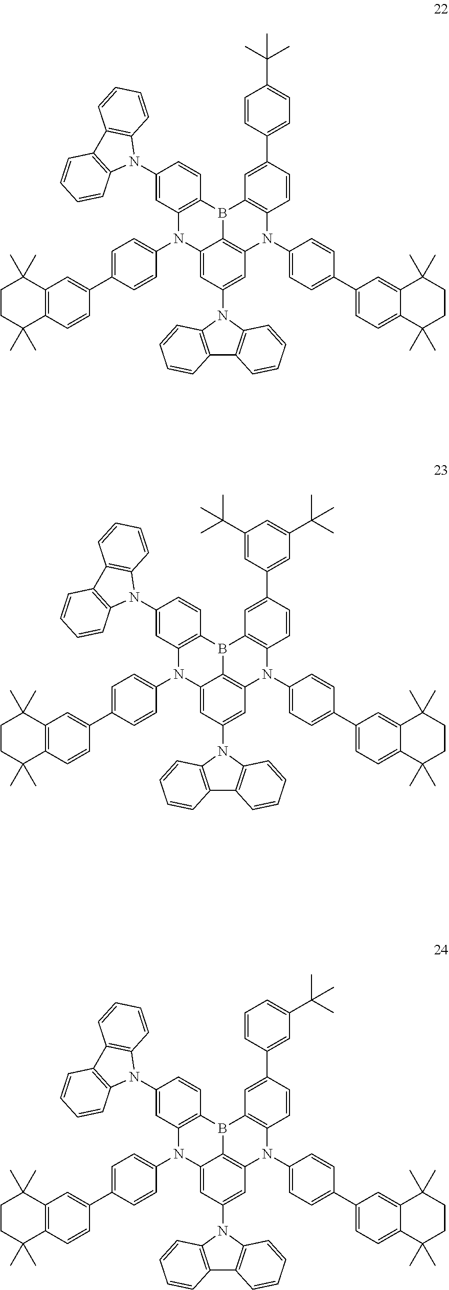

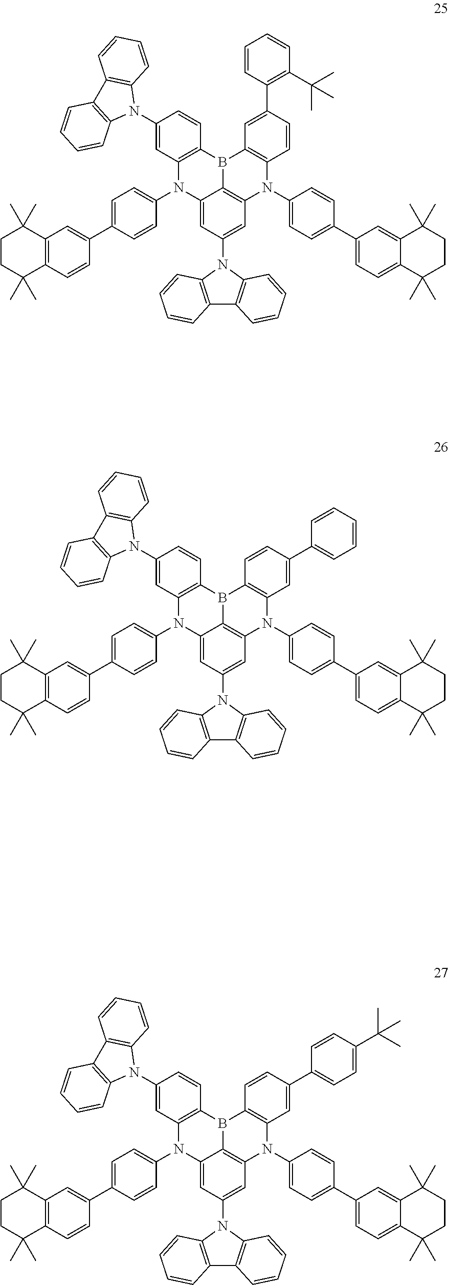

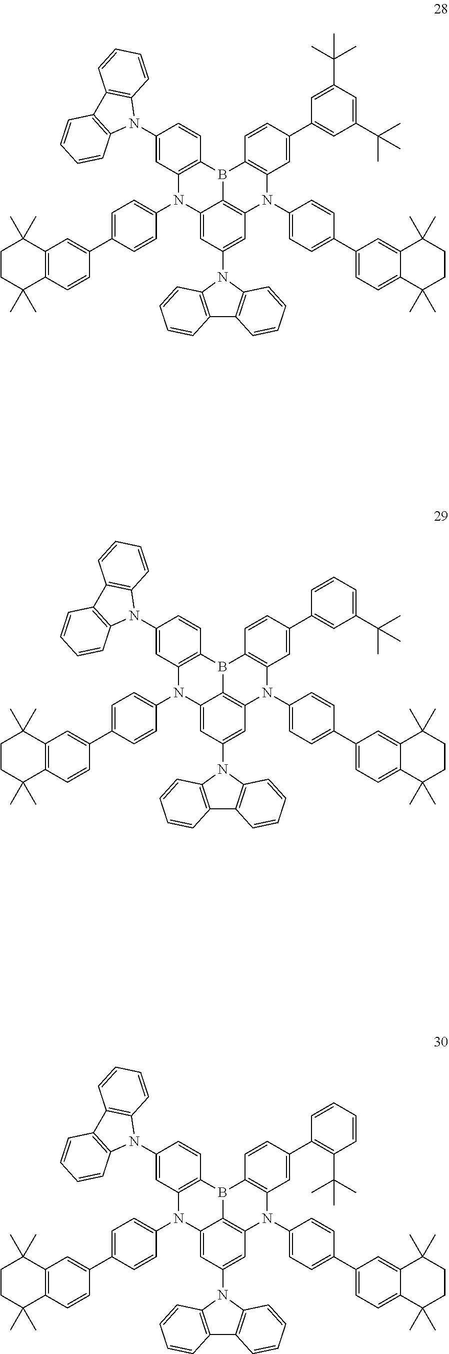

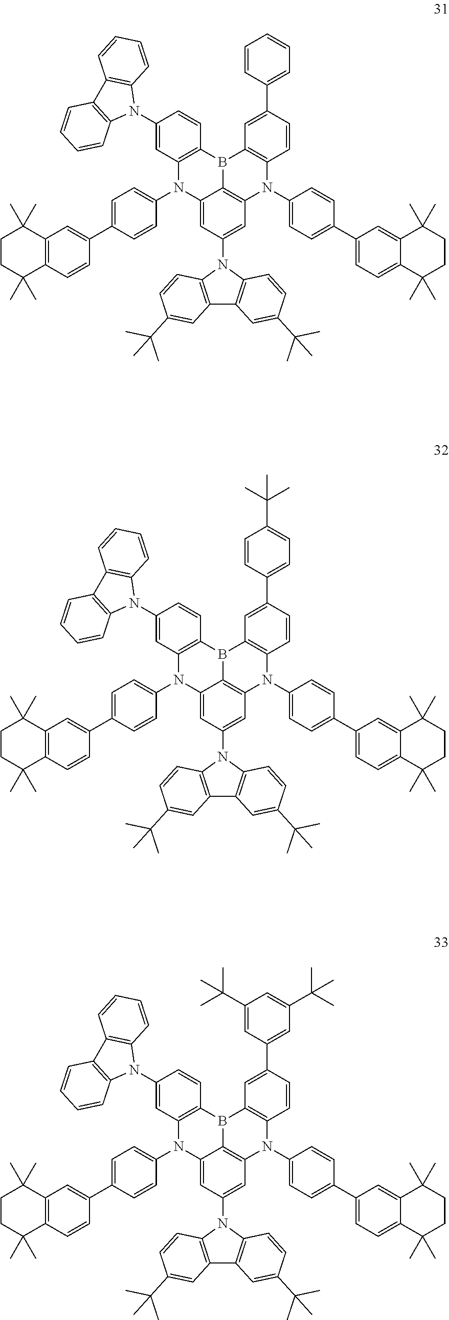

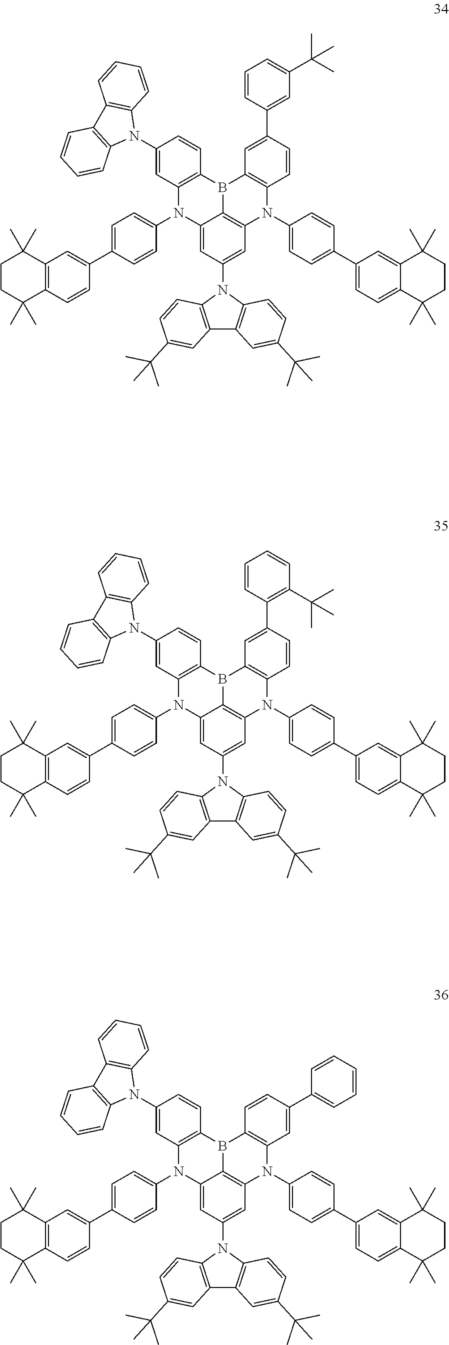

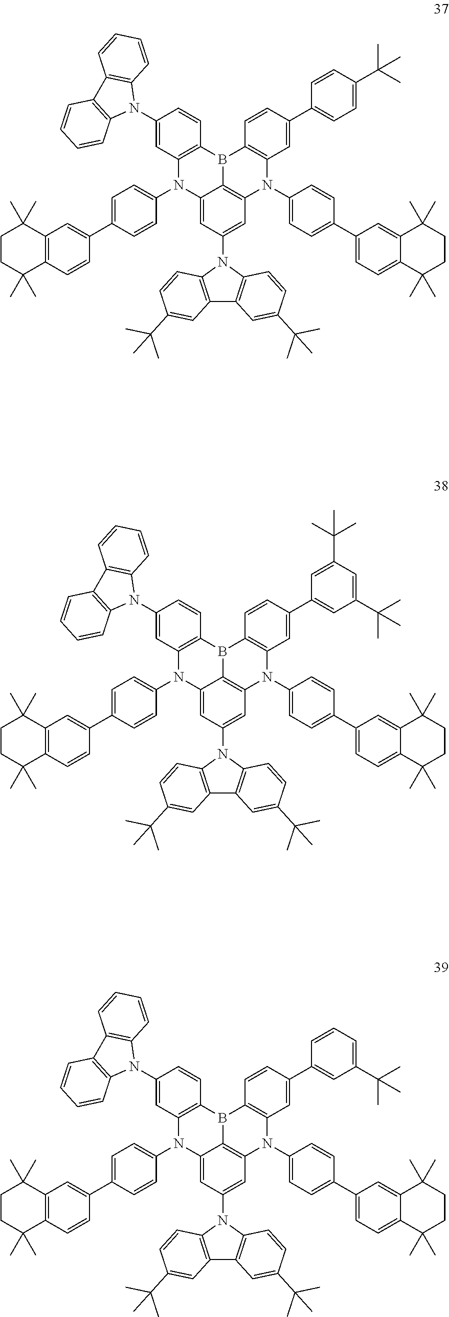

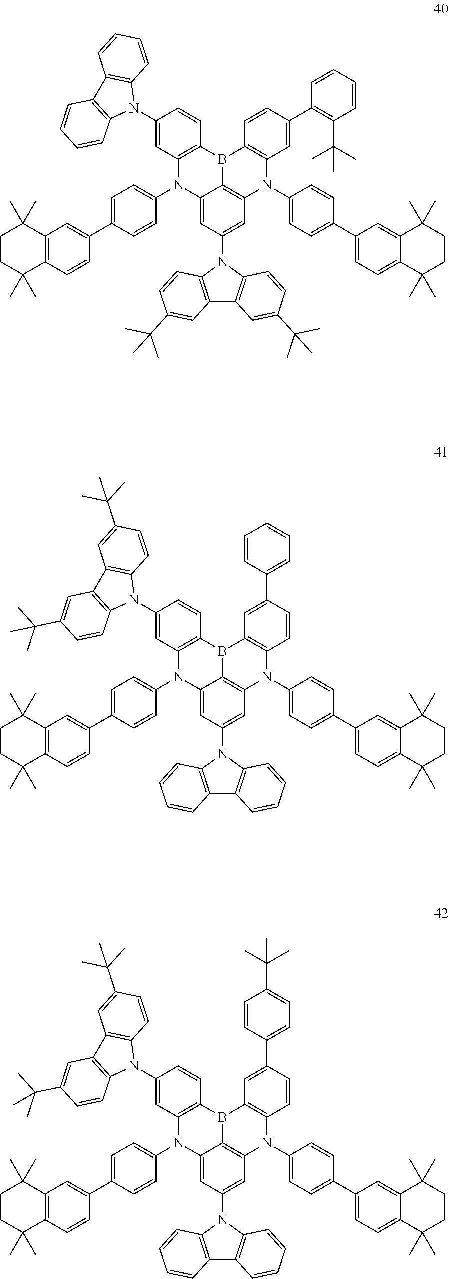

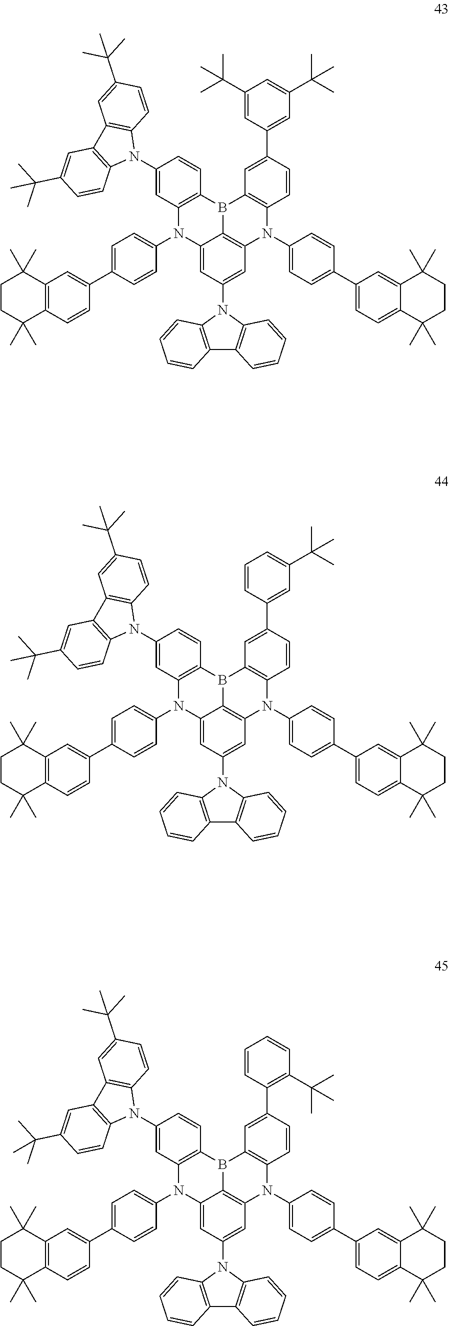

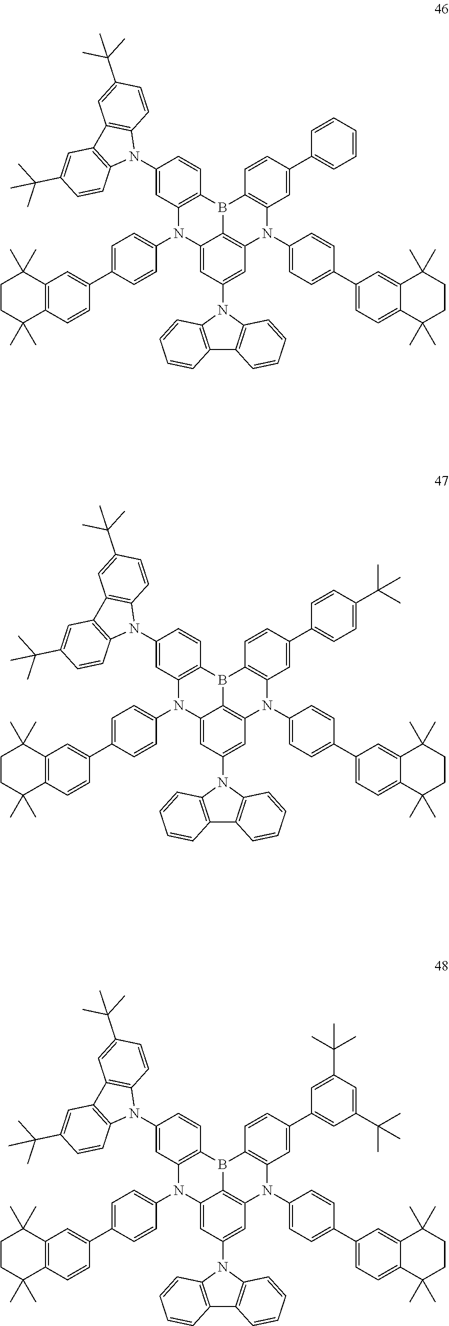

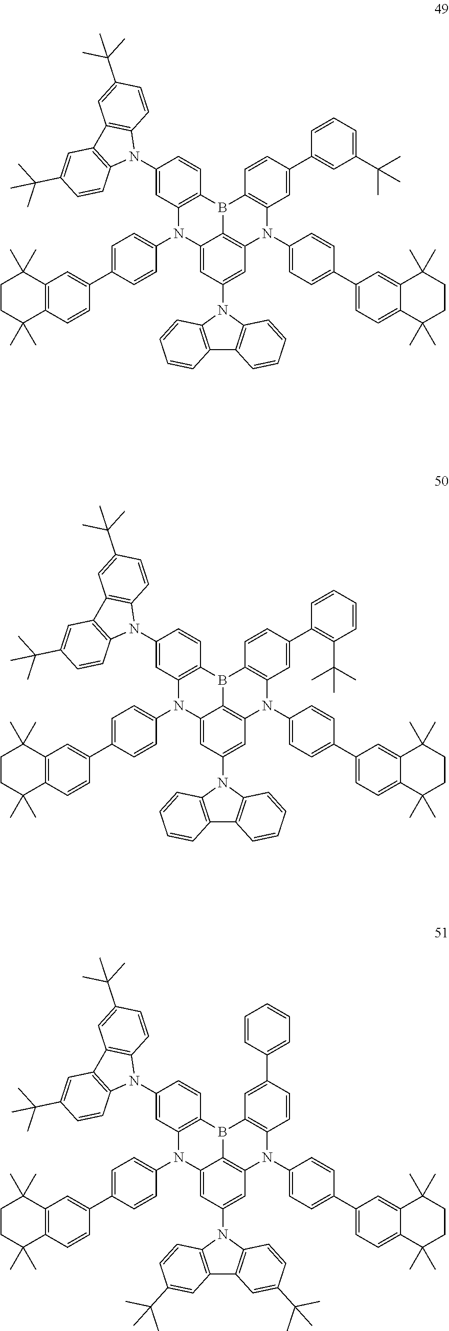

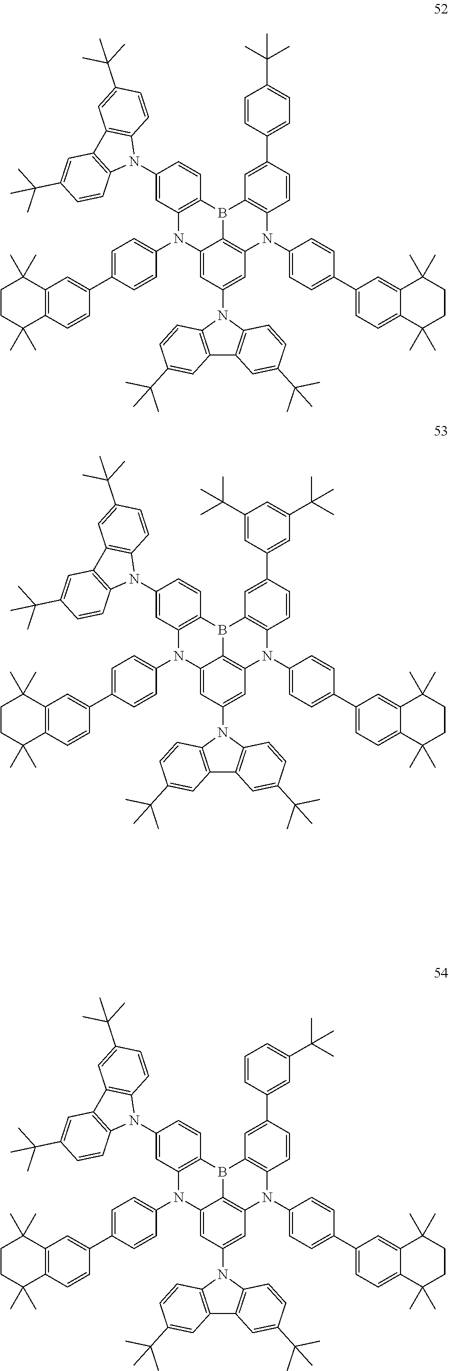

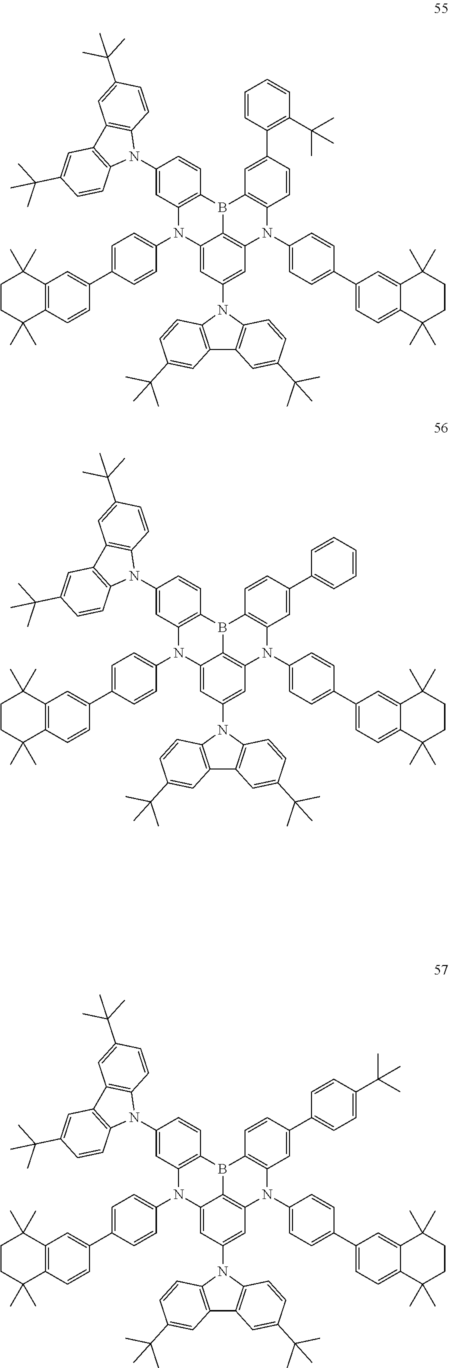

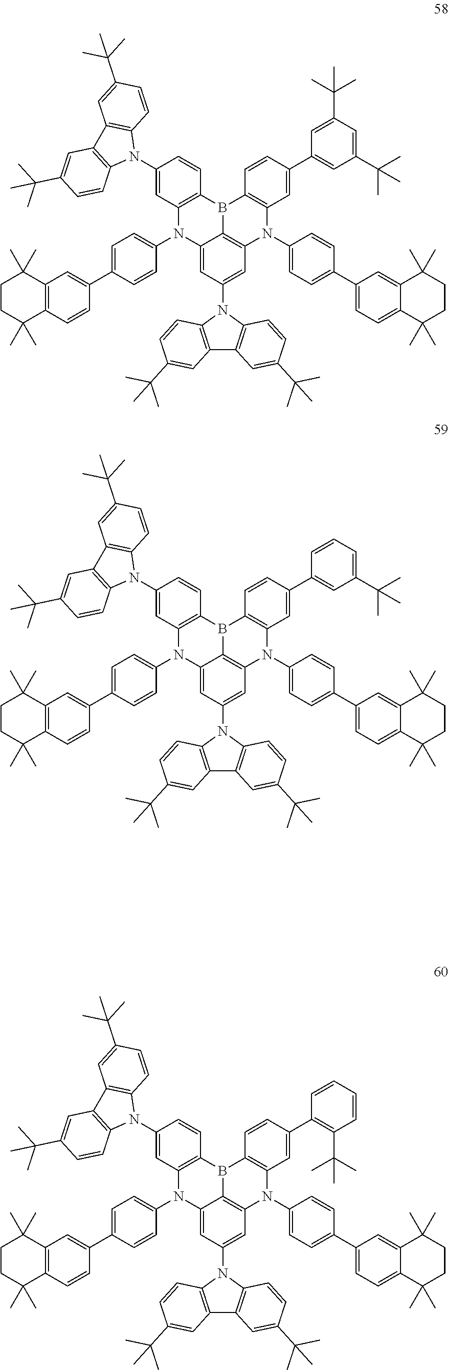

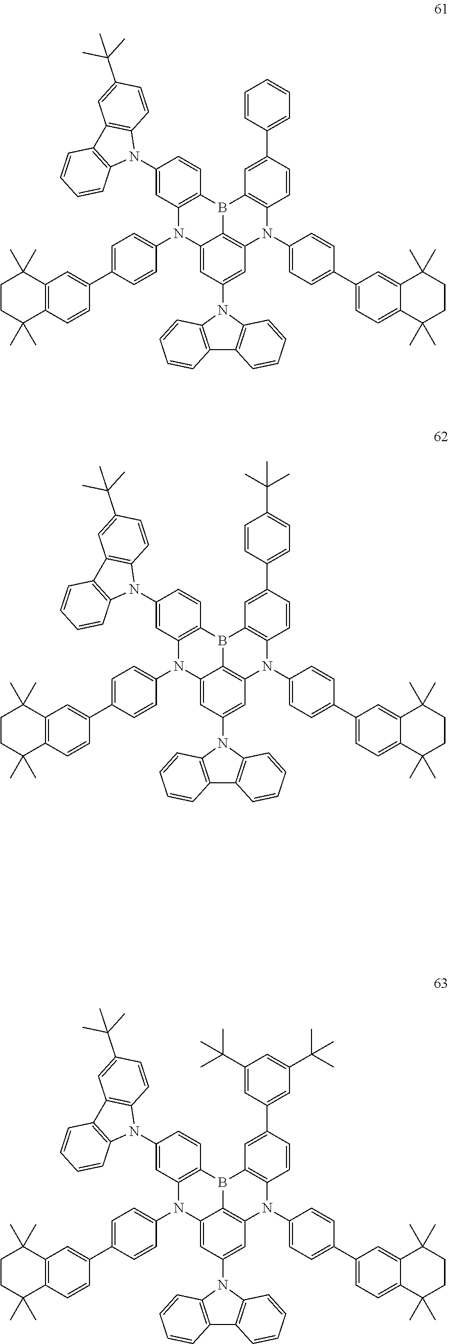

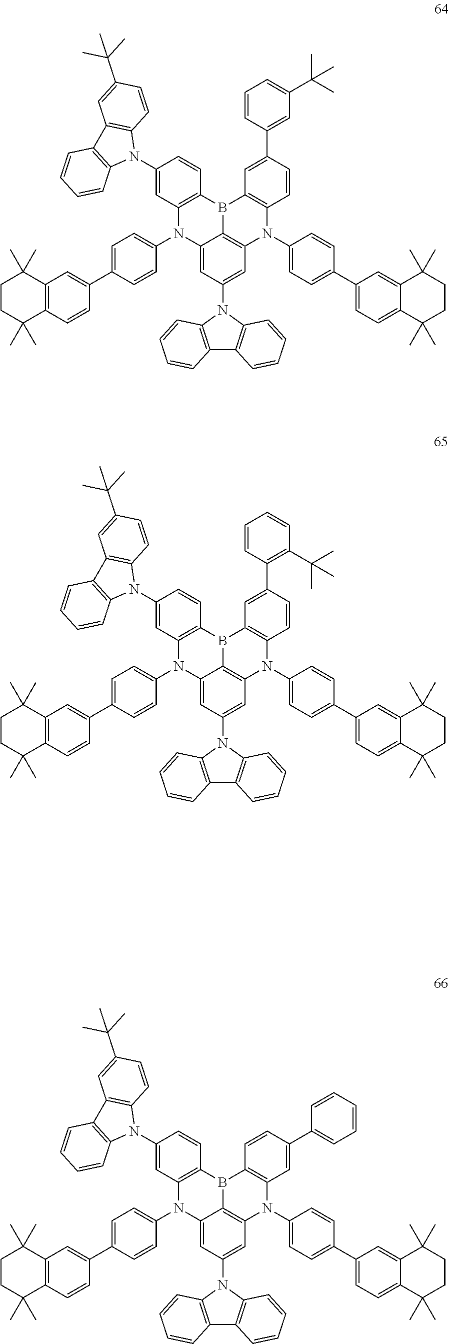

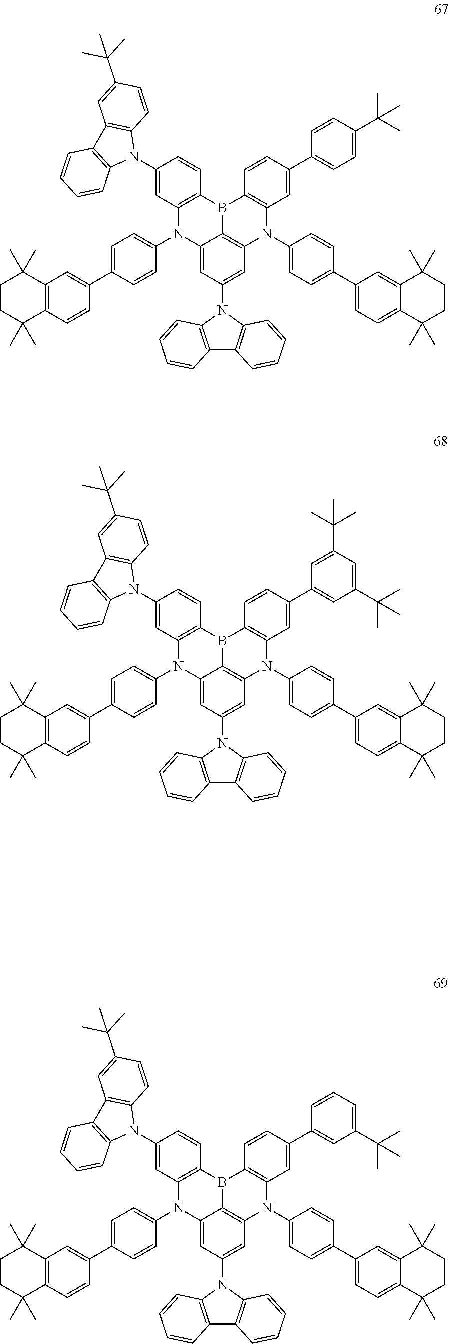

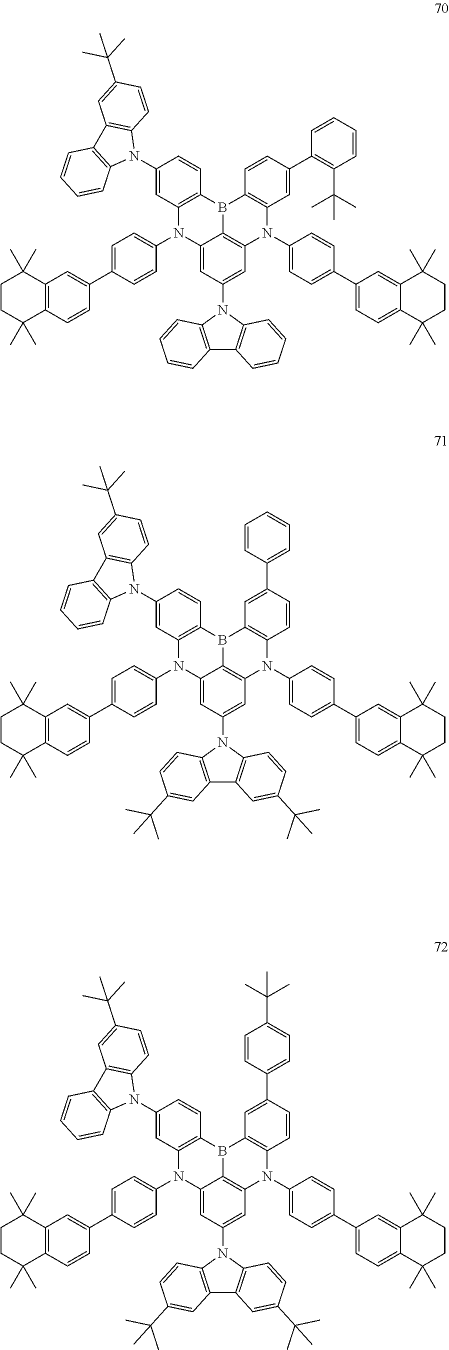

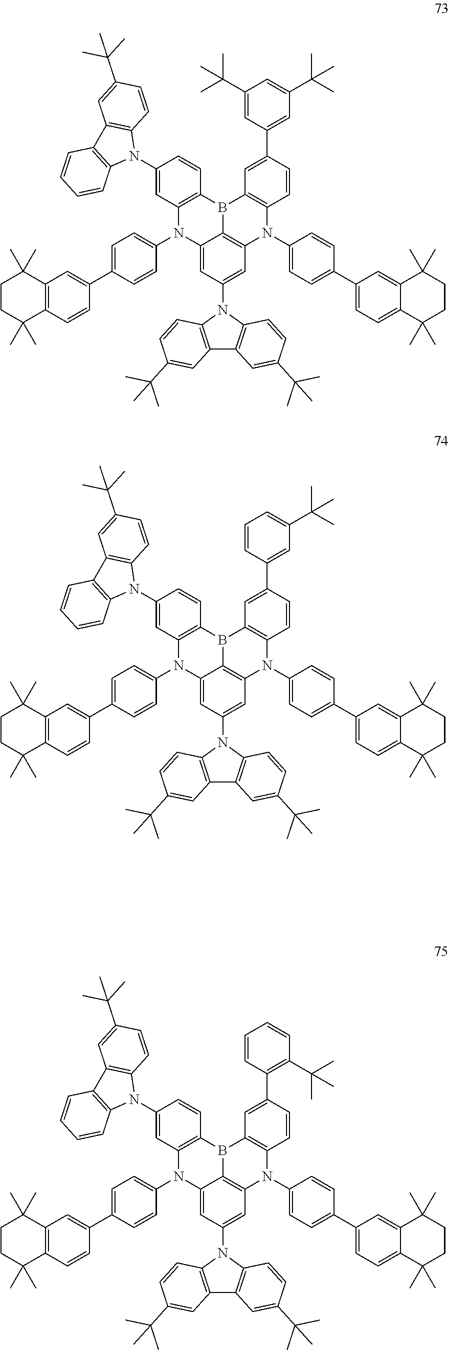

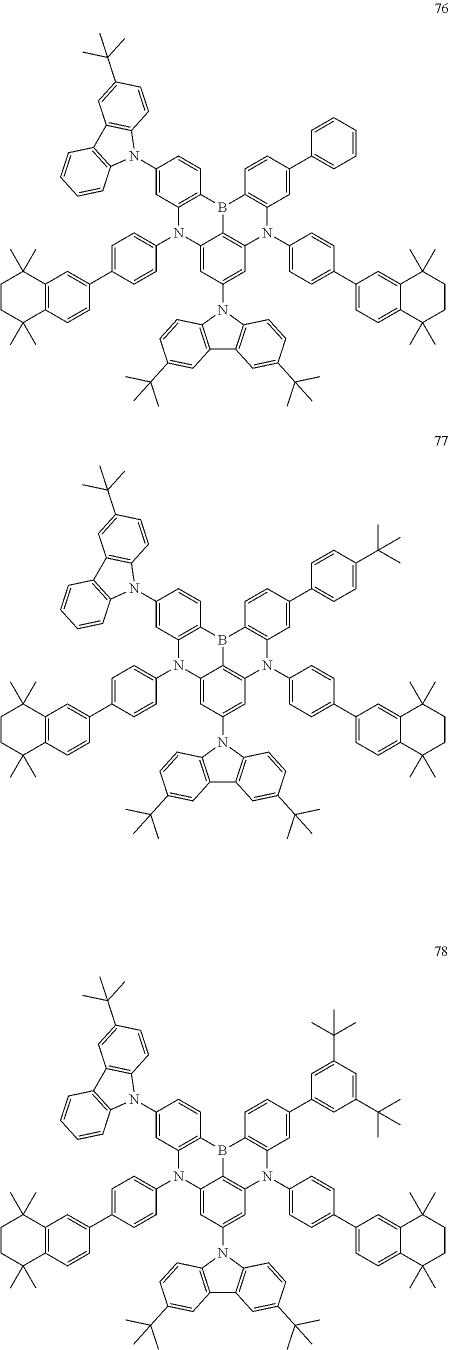

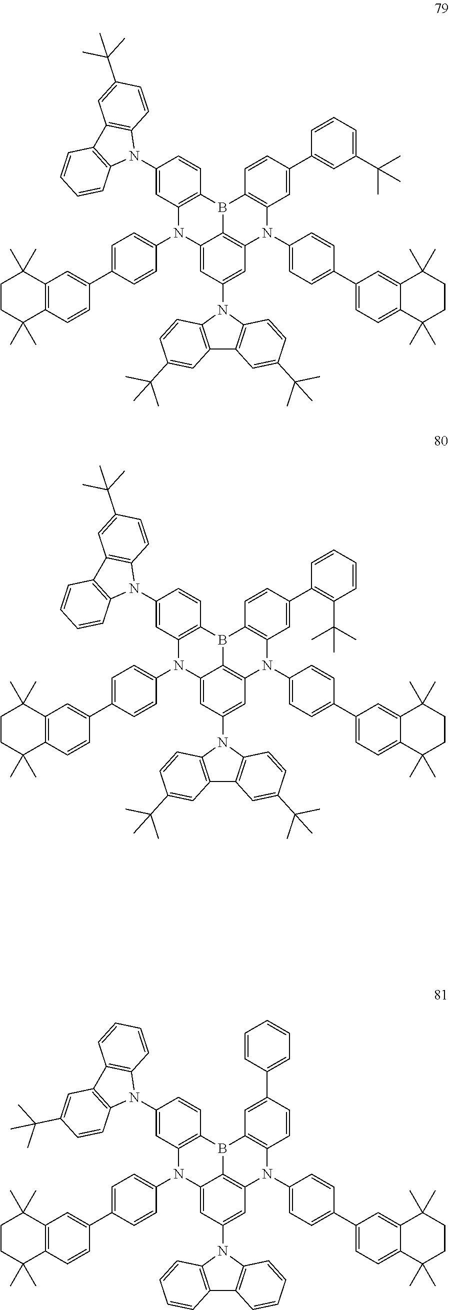

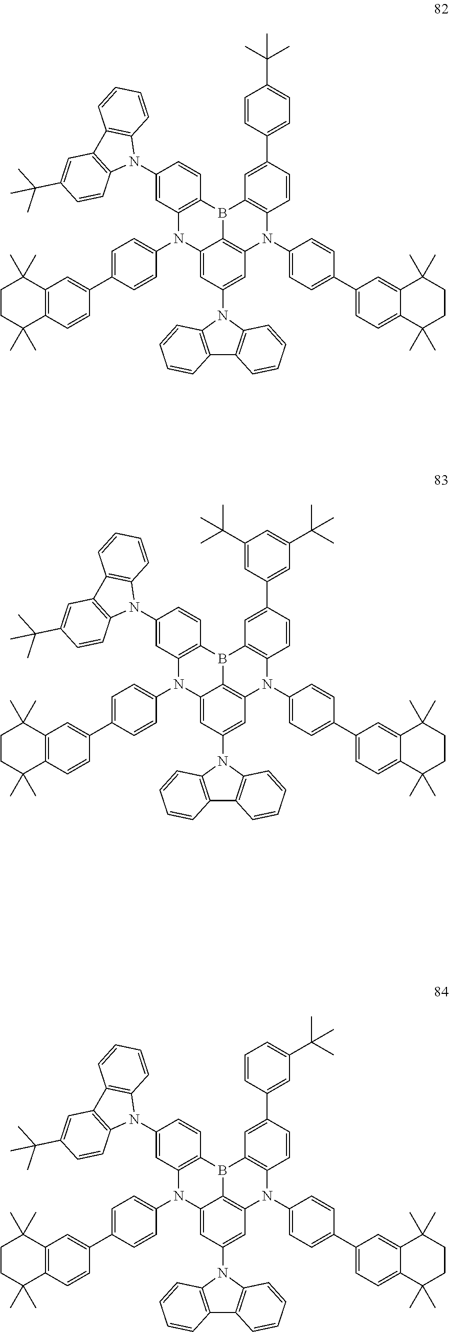

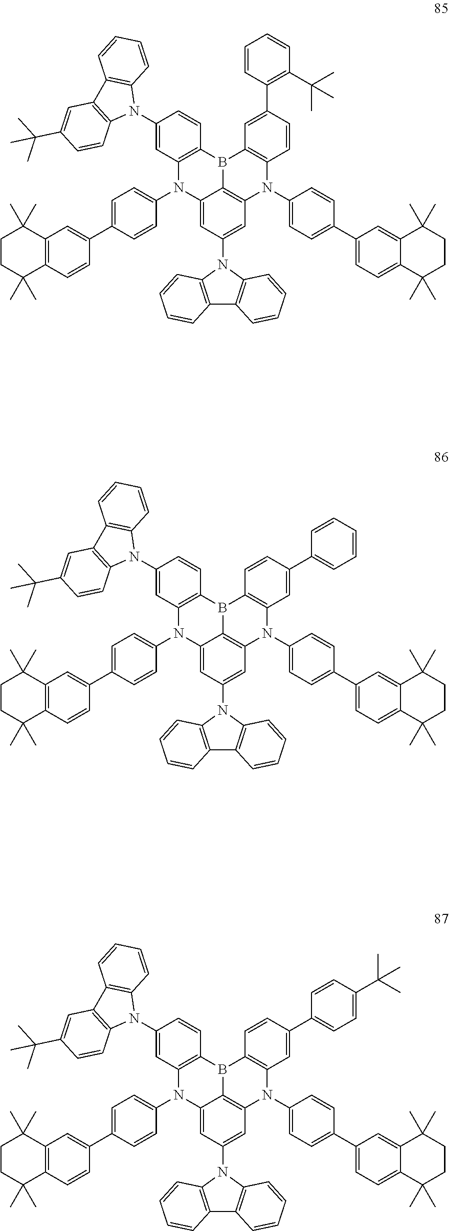

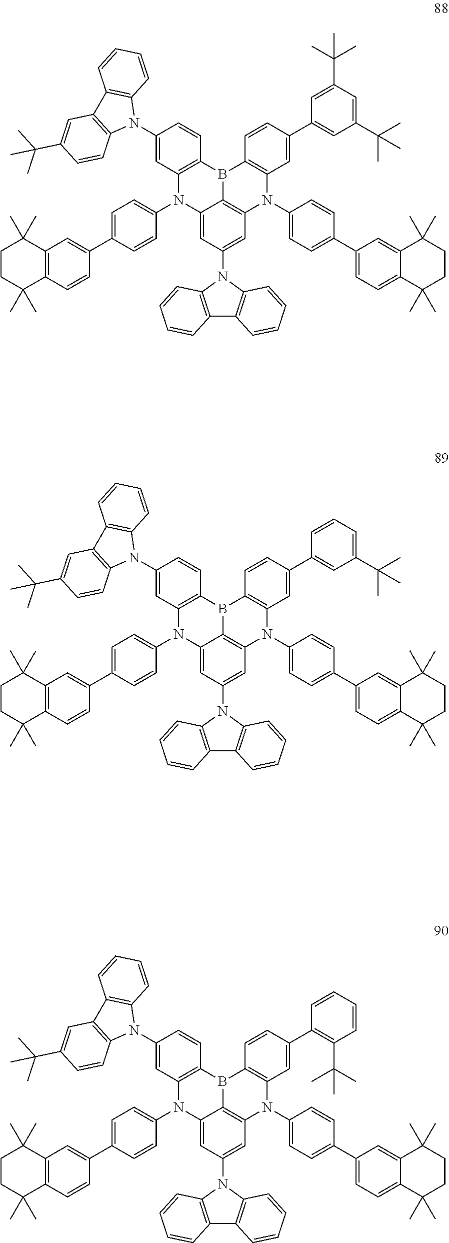

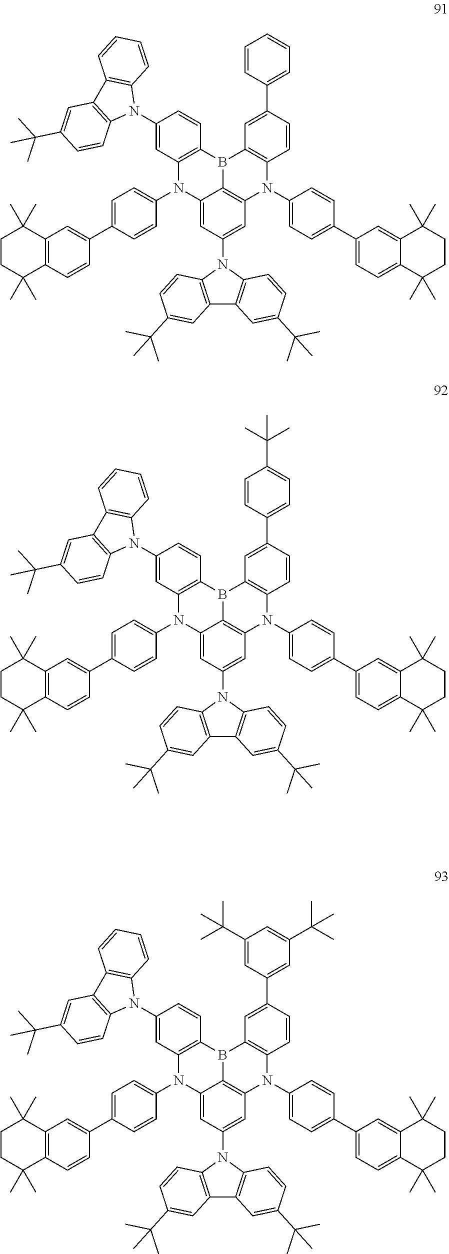

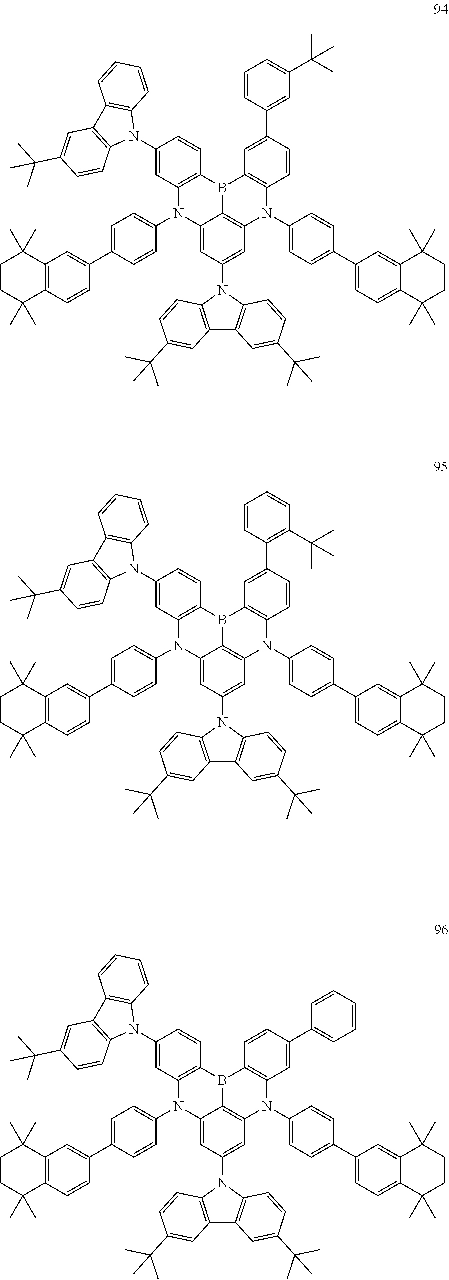

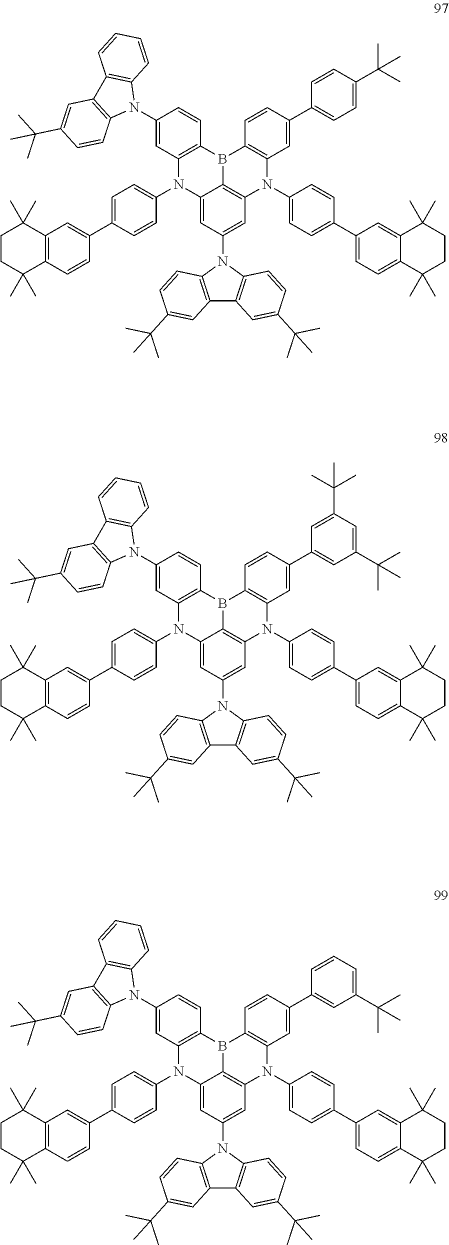

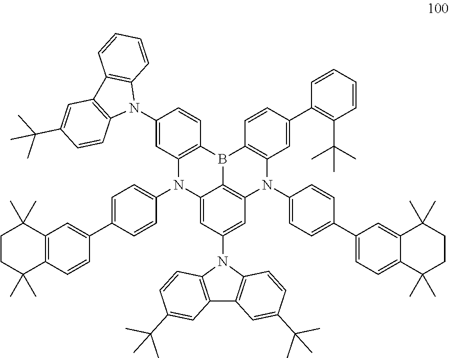 [SMILES: CC(C)(C)c1ccc2c(c1)c1ccccc1n2-c1ccc2c(c1)N(c1ccc(-c3ccc4c(c3)C(C)(C)CCC4(C)C)cc1)c1cc(-n3c4ccc(C(C)(C)C)cc4c4cc(C(C)(C)C)ccc43)cc3c1B2c1ccc(-c2ccccc2C(C)(C)C)cc1N3c1ccc(-c2ccc3c(c2)C(C)(C)CCC3(C)C)cc1]

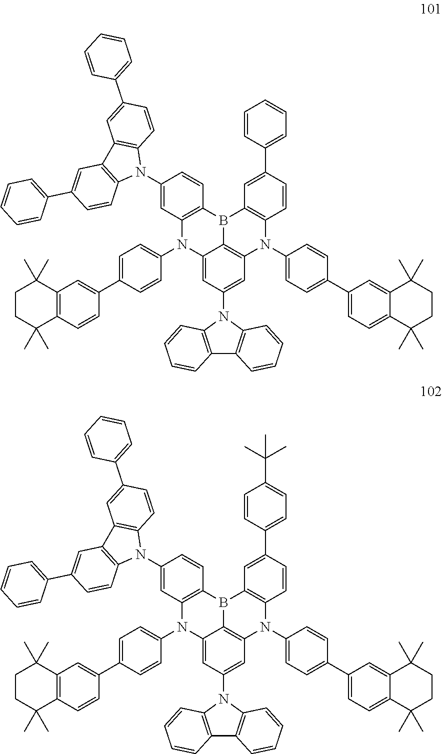 [SMILES: CC1(C)CCC(C)(C)c2cc(-c3ccc(N4c5ccc(-c6ccccc6)cc5B5c6ccc(-n7c8ccc(-c9ccccc9)cc8c8cc(-c9ccccc9)ccc87)cc6N(c6ccc(-c7ccc8c(c7)C(C)(C)CCC8(C)C)cc6)c6cc(-n7c8ccccc8c8ccccc87)cc4c65)cc3)ccc21 ; CC(C)(C)c1ccc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CCC6(C)C)cc4)c4cc(-n5c6ccccc6c6ccccc65)cc(c42)N3c2ccc(-c3ccc4c(c3)C(C)(C)CCC4(C)C)cc2)cc1]

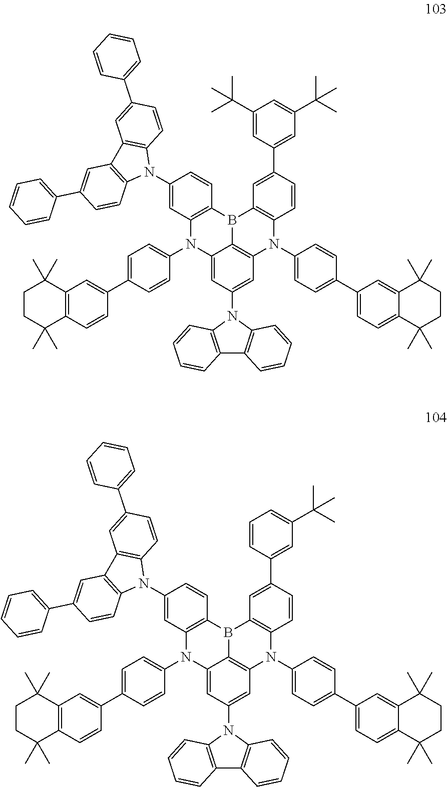

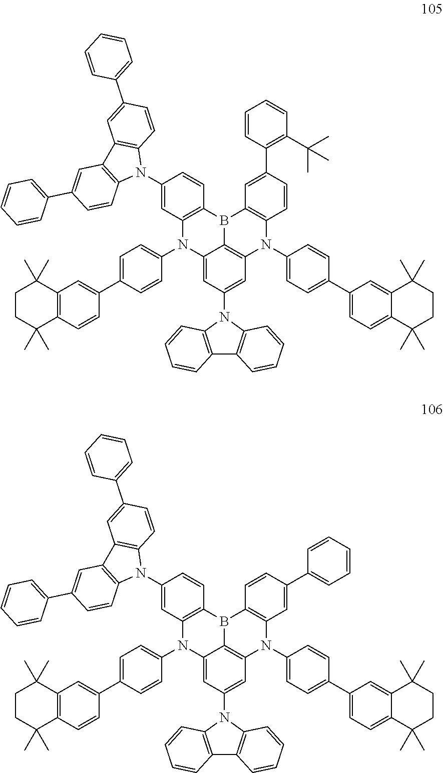 [SMILES: CC(C)(C)c1ccccc1-c1ccc2c(c1)B1c3ccc(-n4c5ccc(-c6ccccc6)cc5c5cc(-c6ccccc6)ccc54)cc3N(c3ccc(-c4ccc5c(c4)C(C)(C)CCC5(C)C)cc3)c3cc(-n4c5ccccc5c5ccccc54)cc(c31)N2c1ccc(-c2ccc3c(c2)C(C)(C)CCC3(C)C)cc1 ; CC1(C)CCC(C)(C)c2cc(-c3ccc(N4c5cc(-c6ccccc6)ccc5B5c6ccc(-n7c8ccc(-c9ccccc9)cc8c8cc(-c9ccccc9)ccc87)cc6N(c6ccc(-c7ccc8c(c7)C(C)(C)CCC8(C)C)cc6)c6cc(-n7c8ccccc8c8ccccc87)cc4c65)cc3)ccc21]

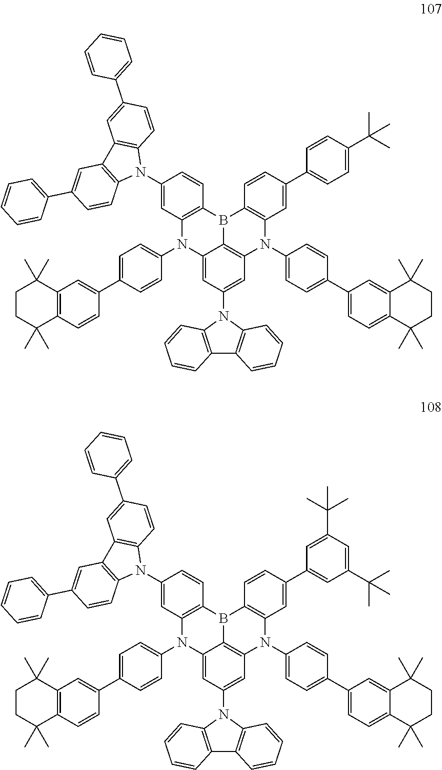 [SMILES: CC(C)(C)c1ccc(-c2ccc3c(c2)N(c2ccc(-c4ccc5c(c4)C(C)(C)CCC5(C)C)cc2)c2cc(-n4c5ccccc5c5ccccc54)cc4c2B3c2ccc(-n3c5ccc(-c6ccccc6)cc5c5cc(-c6ccccc6)ccc53)cc2N4c2ccc(-c3ccc4c(c3)C(C)(C)CCC4(C)C)cc2)cc1 ; CC(C)(C)c1cc(-c2ccc3c(c2)N(c2ccc(-c4ccc5c(c4)C(C)(C)CCC5(C)C)cc2)c2cc(-n4c5ccccc5c5ccccc54)cc4c2B3c2ccc(-n3c5ccc(-c6ccccc6)cc5c5cc(-c6ccccc6)ccc53)cc2N4c2ccc(-c3ccc4c(c3)C(C)(C)CCC4(C)C)cc2)cc(C(C)(C)C)c1]

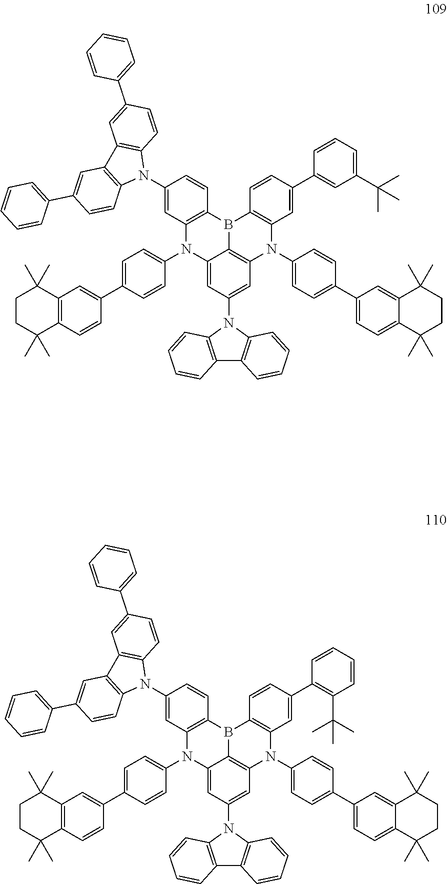 [SMILES: CC(C)(C)c1cccc(-c2ccc3c(c2)N(c2ccc(-c4ccc5c(c4)C(C)(C)CCC5(C)C)cc2)c2cc(-n4c5ccccc5c5ccccc54)cc4c2B3c2ccc(-n3c5ccc(-c6ccccc6)cc5c5cc(-c6ccccc6)ccc53)cc2N4c2ccc(-c3ccc4c(c3)C(C)(C)CCC4(C)C)cc2)c1 ; CC(C)(C)c1ccccc1-c1ccc2c(c1)N(c1ccc(-c3ccc4c(c3)C(C)(C)CCC4(C)C)cc1)c1cc(-n3c4ccccc4c4ccccc43)cc3c1B2c1ccc(-n2c4ccc(-c5ccccc5)cc4c4cc(-c5ccccc5)ccc42)cc1N3c1ccc(-c2ccc3c(c2)C(C)(C)CCC3(C)C)cc1]

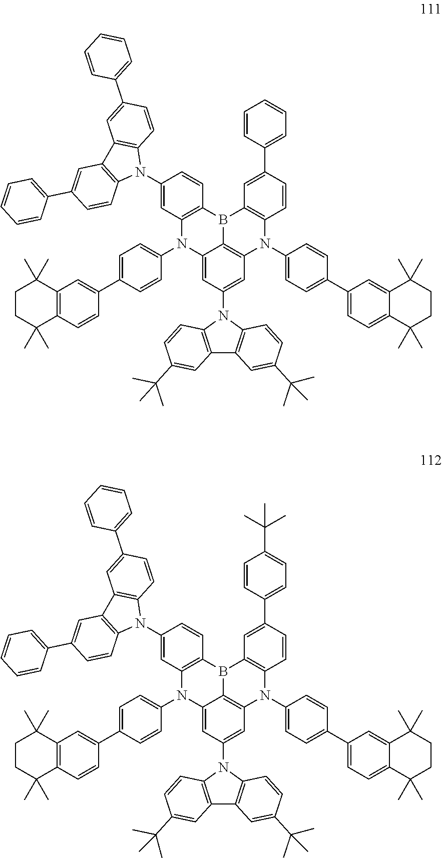 [SMILES: CC(C)(C)c1ccc2c(c1)c1cc(C(C)(C)C)ccc1n2-c1cc2c3c(c1)N(c1ccc(-c4ccc5c(c4)C(C)(C)CCC5(C)C)cc1)c1cc(-n4c5ccc(-c6ccccc6)cc5c5cc(-c6ccccc6)ccc54)ccc1B3c1cc(-c3ccccc3)ccc1N2c1ccc(-c2ccc3c(c2)C(C)(C)CCC3(C)C)cc1 ; CC(C)(C)c1ccc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CCC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CCC4(C)C)cc2)cc1]

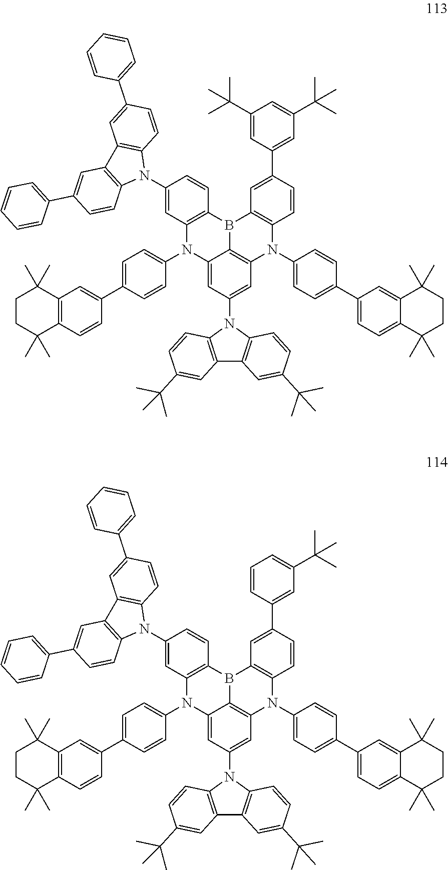 [SMILES: CC(C)(C)c1cc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CCC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CCC4(C)C)cc2)cc(C(C)(C)C)c1 ; CC(C)(C)c1cccc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CCC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CCC4(C)C)cc2)c1]

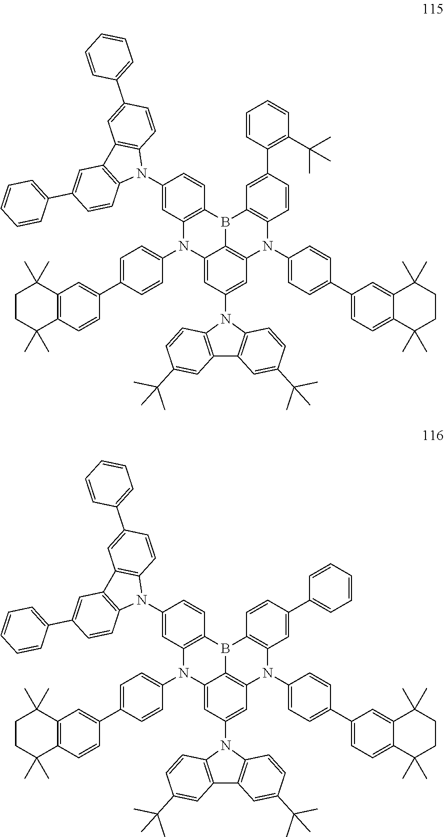 [SMILES: CC(C)(C)c1ccc2c(c1)c1cc(C(C)(C)C)ccc1n2-c1cc2c3c(c1)N(c1ccc(-c4ccc5c(c4)C(C)(C)CCC5(C)C)cc1)c1cc(-n4c5ccc(-c6ccccc6)cc5c5cc(-c6ccccc6)ccc54)ccc1B3c1cc(-c3ccccc3C(C)(C)C)ccc1N2c1ccc(-c2ccc3c(c2)C(C)(C)CCC3(C)C)cc1 ; CC(C)(C)c1ccc2c(c1)c1cc(C(C)(C)C)ccc1n2-c1cc2c3c(c1)N(c1ccc(-c4ccc5c(c4)C(C)(C)CCC5(C)C)cc1)c1cc(-n4c5ccc(-c6ccccc6)cc5c5cc(-c6ccccc6)ccc54)ccc1B3c1ccc(-c3ccccc3)cc1N2c1ccc(-c2ccc3c(c2)C(C)(C)CCC3(C)C)cc1]

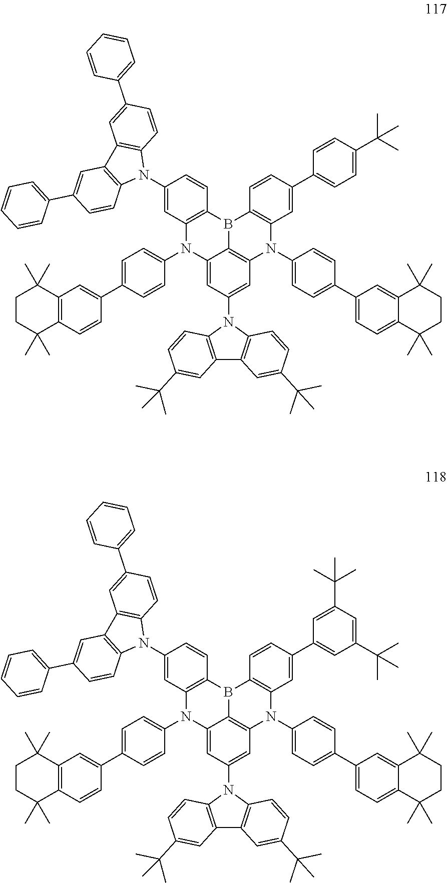 [SMILES: CC(C)(C)c1ccc(-c2ccc3c(c2)N(c2ccc(-c4ccc5c(c4)C(C)(C)CCC5(C)C)cc2)c2cc(-n4c5ccc(C(C)(C)C)cc5c5cc(C(C)(C)C)ccc54)cc4c2B3c2ccc(-n3c5ccc(-c6ccccc6)cc5c5cc(-c6ccccc6)ccc53)cc2N4c2ccc(-c3ccc4c(c3)C(C)(C)CCC4(C)C)cc2)cc1 ; CC(C)(C)c1cc(-c2ccc3c(c2)N(c2ccc(-c4ccc5c(c4)C(C)(C)CCC5(C)C)cc2)c2cc(-n4c5ccc(C(C)(C)C)cc5c5cc(C(C)(C)C)ccc54)cc4c2B3c2ccc(-n3c5ccc(-c6ccccc6)cc5c5cc(-c6ccccc6)ccc53)cc2N4c2ccc(-c3ccc4c(c3)C(C)(C)CCC4(C)C)cc2)cc(C(C)(C)C)c1]

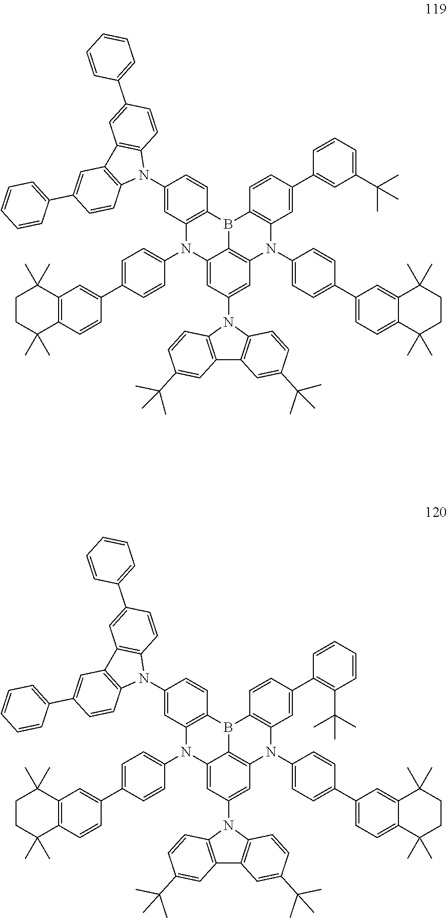 [SMILES: CC(C)(C)c1cccc(-c2ccc3c(c2)N(c2ccc(-c4ccc5c(c4)C(C)(C)CCC5(C)C)cc2)c2cc(-n4c5ccc(C(C)(C)C)cc5c5cc(C(C)(C)C)ccc54)cc4c2B3c2ccc(-n3c5ccc(-c6ccccc6)cc5c5cc(-c6ccccc6)ccc53)cc2N4c2ccc(-c3ccc4c(c3)C(C)(C)CCC4(C)C)cc2)c1 ; CC(C)(C)c1ccc2c(c1)c1cc(C(C)(C)C)ccc1n2-c1cc2c3c(c1)N(c1ccc(-c4ccc5c(c4)C(C)(C)CCC5(C)C)cc1)c1cc(-n4c5ccc(-c6ccccc6)cc5c5cc(-c6ccccc6)ccc54)ccc1B3c1ccc(-c3ccccc3C(C)(C)C)cc1N2c1ccc(-c2ccc3c(c2)C(C)(C)CCC3(C)C)cc1]

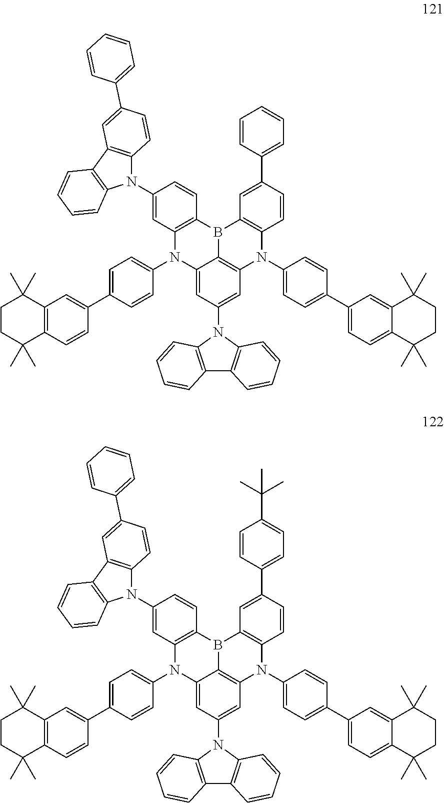 [SMILES: CC1(C)CCC(C)(C)c2cc(-c3ccc(N4c5ccc(-c6ccccc6)cc5B5c6ccc(-n7c8ccccc8c8cc(-c9ccccc9)ccc87)cc6N(c6ccc(-c7ccc8c(c7)C(C)(C)CCC8(C)C)cc6)c6cc(-n7c8ccccc8c8ccccc87)cc4c65)cc3)ccc21 ; CC(C)(C)c1ccc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccccc6c6ccccc65)cc(c42)N3c2ccc(-c3ccc4c(c3)C(C)(C)CCC4(C)C)cc2)cc1]

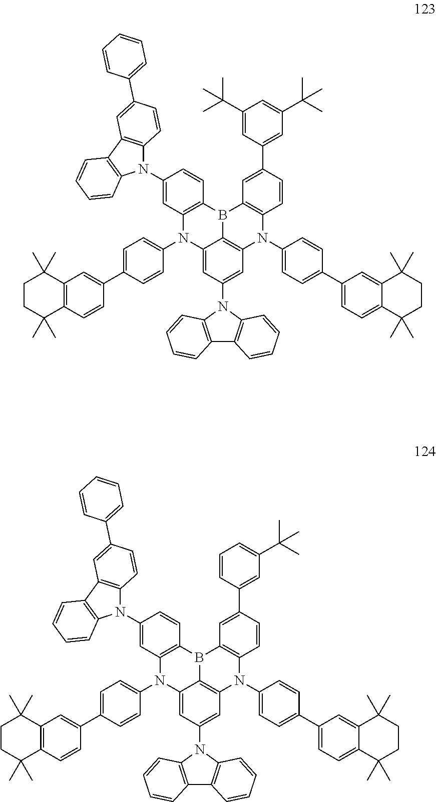

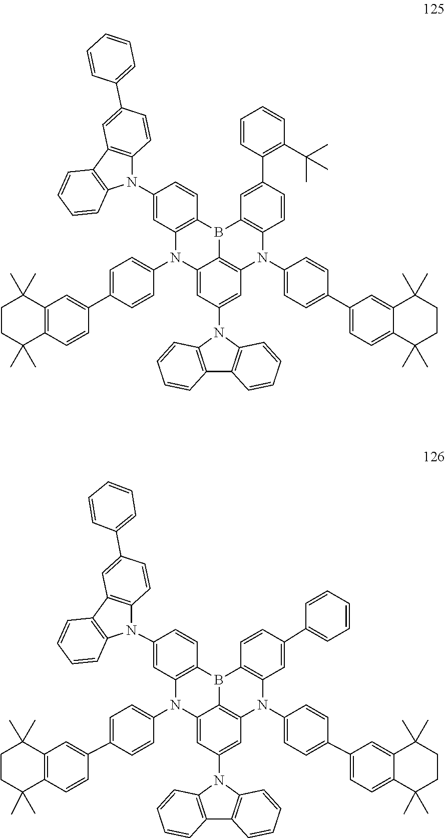 [SMILES: CC(C)(C)c1ccccc1-c1ccc2c(c1)B1c3ccc(-n4c5ccccc5c5cc(-c6ccccc6)ccc54)cc3N(c3ccc(-c4ccc5c(c4)C(C)(C)CCC5(C)C)cc3)c3cc(-n4c5ccccc5c5ccccc54)cc(c31)N2c1ccc(-c2ccc3c(c2)C(C)(C)CCC3(C)C)cc1 ; CC1(C)CCC(C)(C)c2cc(-c3ccc(N4c5cc(-c6ccccc6)ccc5B5c6ccc(-n7c8ccccc8c8cc(-c9ccccc9)ccc87)cc6N(c6ccc(-c7ccc8c(c7)C(C)(C)CCC8(C)C)cc6)c6cc(-n7c8ccccc8c8ccccc87)cc4c65)cc3)ccc21]

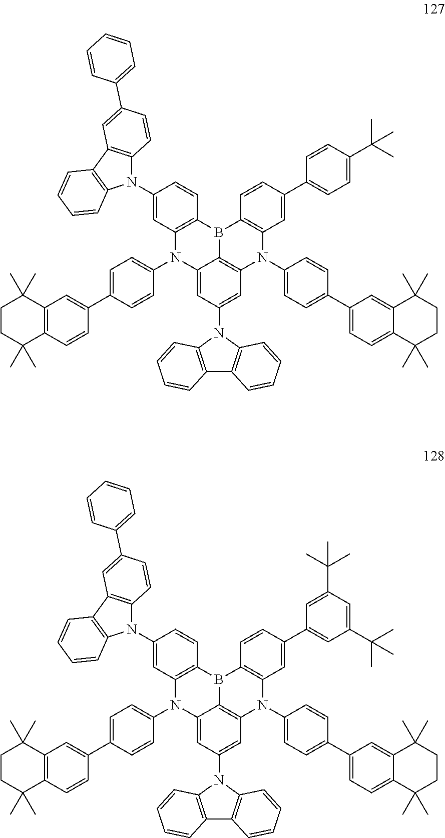 [SMILES: CC(C)(C)c1ccc(-c2ccc3c(c2)N(c2ccc(-c4ccc5c(c4)C(C)(C)CCC5(C)C)cc2)c2cc(-n4c5ccccc5c5ccccc54)cc4c2B3c2ccc(-n3c5ccccc5c5cc(-c6ccccc6)ccc53)cc2N4c2ccc(-c3ccc4c(c3)C(C)(C)CCC4(C)C)cc2)cc1 ; CC(C)(C)c1cc(-c2ccc3c(c2)N(c2ccc(-c4ccc5c(c4)C(C)(C)CCC5(C)C)cc2)c2cc(-n4c5ccccc5c5ccccc54)cc4c2B3c2ccc(-n3c5ccccc5c5cc(-c6ccccc6)ccc53)cc2N4c2ccc(-c3ccc4c(c3)C(C)(C)CCC4(C)C)cc2)cc(C(C)(C)C)c1]

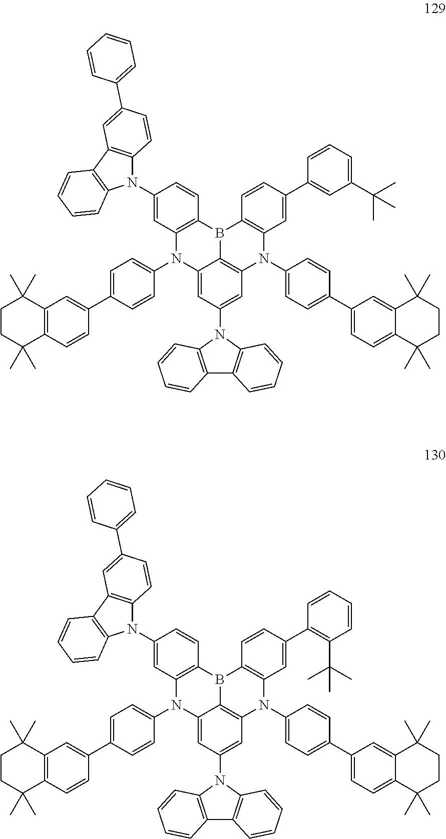 [SMILES: CC(C)(C)c1cccc(-c2ccc3c(c2)N(c2ccc(-c4ccc5c(c4)C(C)(C)CCC5(C)C)cc2)c2cc(-n4c5ccccc5c5ccccc54)cc4c2B3c2ccc(-n3c5ccccc5c5cc(-c6ccccc6)ccc53)cc2N4c2ccc(-c3ccc4c(c3)C(C)(C)CCC4(C)C)cc2)c1 ; CC(C)(C)c1ccccc1-c1ccc2c(c1)N(c1ccc(-c3ccc4c(c3)C(C)(C)CCC4(C)C)cc1)c1cc(-n3c4ccccc4c4ccccc43)cc3c1B2c1ccc(-n2c4ccccc4c4cc(-c5ccccc5)ccc42)cc1N3c1ccc(-c2ccc3c(c2)C(C)(C)CCC3(C)C)cc1]

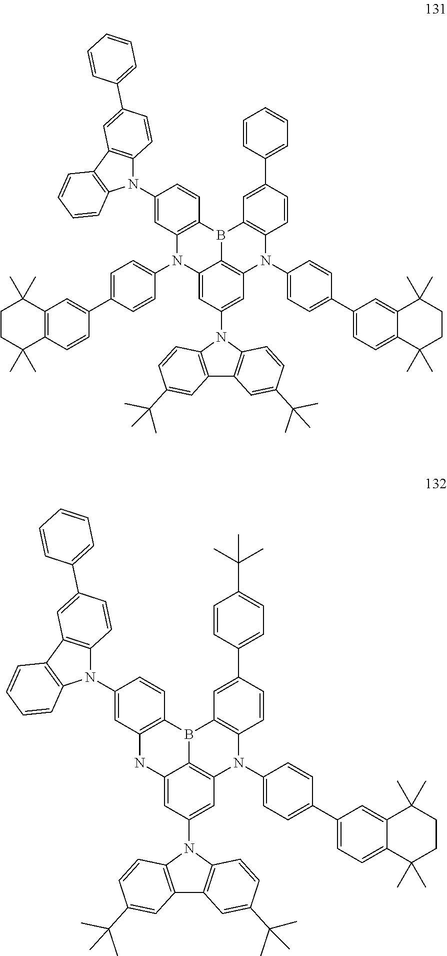

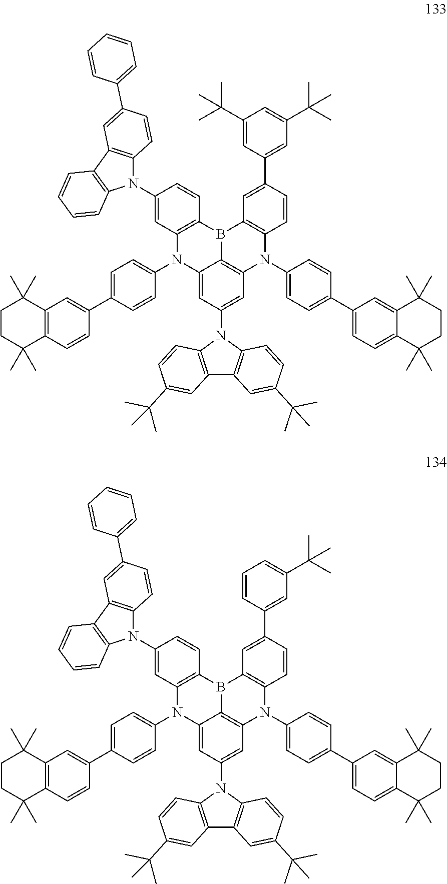 [SMILES: CC(C)(C)c1cc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CCC4(C)C)cc2)cc(C(C)(C)C)c1 ; CC(C)(C)c1cccc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CCC4(C)C)cc2)c1]

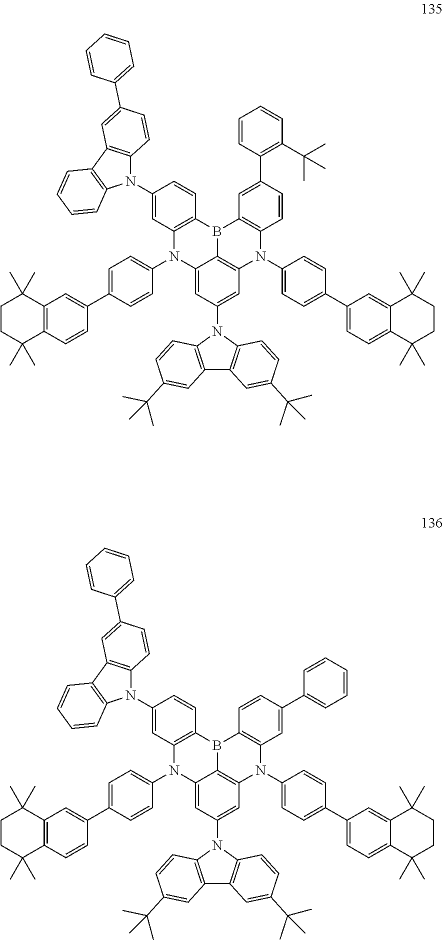 [SMILES: CC(C)(C)c1ccc2c(c1)c1cc(C(C)(C)C)ccc1n2-c1cc2c3c(c1)N(c1ccc(-c4ccc5c(c4)C(C)(C)CCC5(C)C)cc1)c1cc(-n4c5ccccc5c5cc(-c6ccccc6)ccc54)ccc1B3c1cc(-c3ccccc3C(C)(C)C)ccc1N2c1ccc(-c2ccc3c(c2)C(C)(C)CCC3(C)C)cc1 ; CC(C)(C)c1ccc2c(c1)c1cc(C(C)(C)C)ccc1n2-c1cc2c3c(c1)N(c1ccc(-c4ccc5c(c4)C(C)(C)CCC5(C)C)cc1)c1cc(-n4c5ccccc5c5cc(-c6ccccc6)ccc54)ccc1B3c1ccc(-c3ccccc3)cc1N2c1ccc(-c2ccc3c(c2)C(C)(C)CCC3(C)C)cc1]

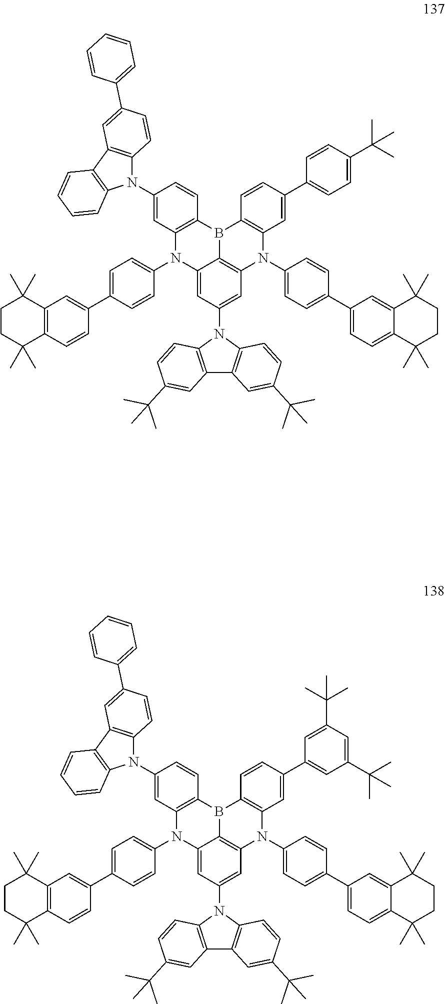 [SMILES: CC(C)(C)c1ccc(-c2ccc3c(c2)N(c2ccc(-c4ccc5c(c4)C(C)(C)CCC5(C)C)cc2)c2cc(-n4c5ccc(C(C)(C)C)cc5c5cc(C(C)(C)C)ccc54)cc4c2B3c2ccc(-n3c5ccccc5c5cc(-c6ccccc6)ccc53)cc2N4c2ccc(-c3ccc4c(c3)C(C)(C)CCC4(C)C)cc2)cc1 ; CC(C)(C)c1cc(-c2ccc3c(c2)N(c2ccc(-c4ccc5c(c4)C(C)(C)CCC5(C)C)cc2)c2cc(-n4c5ccc(C(C)(C)C)cc5c5cc(C(C)(C)C)ccc54)cc4c2B3c2ccc(-n3c5ccccc5c5cc(-c6ccccc6)ccc53)cc2N4c2ccc(-c3ccc4c(c3)C(C)(C)CCC4(C)C)cc2)cc(C(C)(C)C)c1]

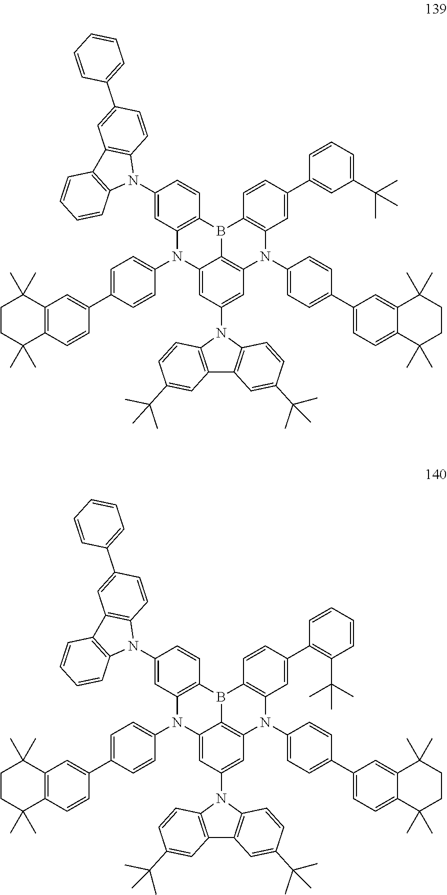 [SMILES: CC(C)(C)c1cccc(-c2ccc3c(c2)N(c2ccc(-c4ccc5c(c4)C(C)(C)CCC5(C)C)cc2)c2cc(-n4c5ccc(C(C)(C)C)cc5c5cc(C(C)(C)C)ccc54)cc4c2B3c2ccc(-n3c5ccccc5c5cc(-c6ccccc6)ccc53)cc2N4c2ccc(-c3ccc4c(c3)C(C)(C)CCC4(C)C)cc2)c1 ; CC(C)(C)c1ccc2c(c1)c1cc(C(C)(C)C)ccc1n2-c1cc2c3c(c1)N(c1ccc(-c4ccc5c(c4)C(C)(C)CCC5(C)C)cc1)c1cc(-n4c5ccccc5c5cc(-c6ccccc6)ccc54)ccc1B3c1ccc(-c3ccccc3C(C)(C)C)cc1N2c1ccc(-c2ccc3c(c2)C(C)(C)CCC3(C)C)cc1]

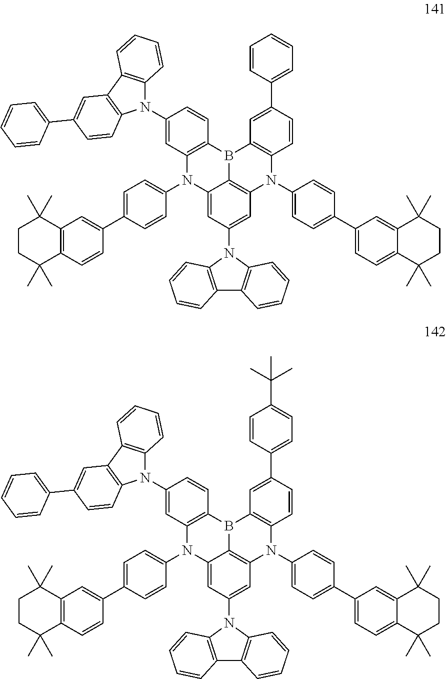 [SMILES: CC1(C)CCC(C)(C)c2cc(-c3ccc(N4c5ccc(-c6ccccc6)cc5B5c6ccc(-n7c8ccccc8c8cc(-c9ccccc9)ccc87)cc6N(c6ccc(-c7ccc8c(c7)C(C)(C)CCC8(C)C)cc6)c6cc(-n7c8ccccc8c8ccccc87)cc4c65)cc3)ccc21 ; CC(C)(C)c1ccc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccccc6c6ccccc65)cc(c42)N3c2ccc(-c3ccc4c(c3)C(C)(C)CCC4(C)C)cc2)cc1]

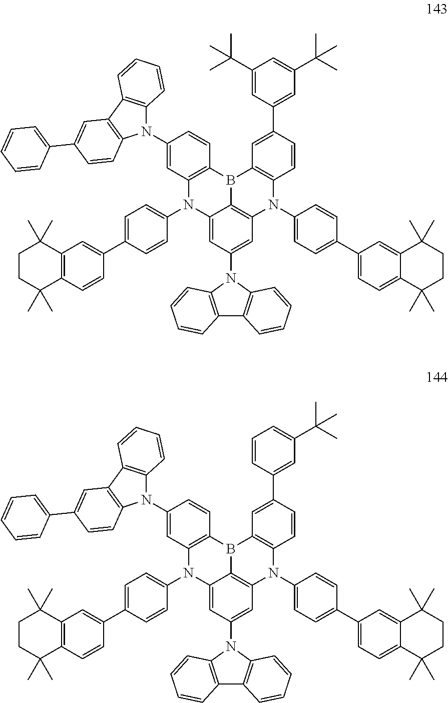 [SMILES: CC(C)(C)c1cc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccccc6c6ccccc65)cc(c42)N3c2ccc(-c3ccc4c(c3)C(C)(C)CCC4(C)C)cc2)cc(C(C)(C)C)c1 ; CC(C)(C)c1cccc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccccc6c6ccccc65)cc(c42)N3c2ccc(-c3ccc4c(c3)C(C)(C)CCC4(C)C)cc2)c1]

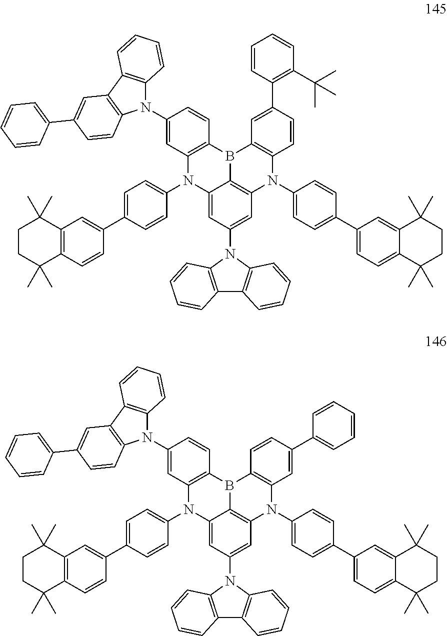 [SMILES: CC(C)(C)c1ccccc1-c1ccc2c(c1)B1c3ccc(-n4c5ccccc5c5cc(-c6ccccc6)ccc54)cc3N(c3ccc(-c4ccc5c(c4)C(C)(C)CCC5(C)C)cc3)c3cc(-n4c5ccccc5c5ccccc54)cc(c31)N2c1ccc(-c2ccc3c(c2)C(C)(C)CCC3(C)C)cc1 ; CC1(C)CCC(C)(C)c2cc(-c3ccc(N4c5cc(-c6ccccc6)ccc5B5c6ccc(-n7c8ccccc8c8cc(-c9ccccc9)ccc87)cc6N(c6ccc(-c7ccc8c(c7)C(C)(C)CCC8(C)C)cc6)c6cc(-n7c8ccccc8c8ccccc87)cc4c65)cc3)ccc21]

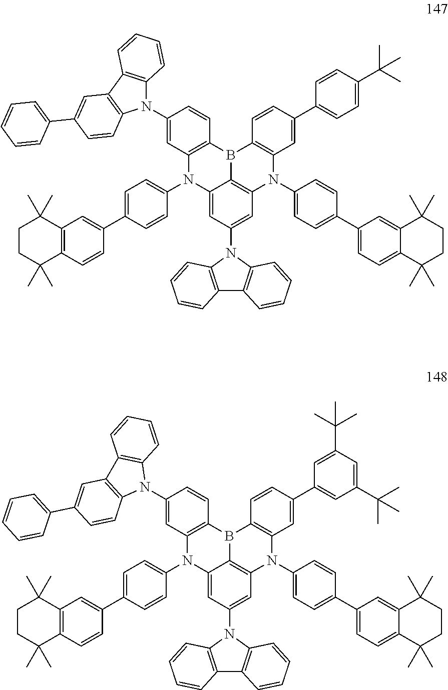 [SMILES: CC(C)(C)c1ccc(-c2ccc3c(c2)N(c2ccc(-c4ccc5c(c4)C(C)(C)CCC5(C)C)cc2)c2cc(-n4c5ccccc5c5ccccc54)cc4c2B3c2ccc(-n3c5ccccc5c5cc(-c6ccccc6)ccc53)cc2N4c2ccc(-c3ccc4c(c3)C(C)(C)CCC4(C)C)cc2)cc1 ; CC(C)(C)c1cc(-c2ccc3c(c2)N(c2ccc(-c4ccc5c(c4)C(C)(C)CCC5(C)C)cc2)c2cc(-n4c5ccccc5c5ccccc54)cc4c2B3c2ccc(-n3c5ccccc5c5cc(-c6ccccc6)ccc53)cc2N4c2ccc(-c3ccc4c(c3)C(C)(C)CCC4(C)C)cc2)cc(C(C)(C)C)c1]

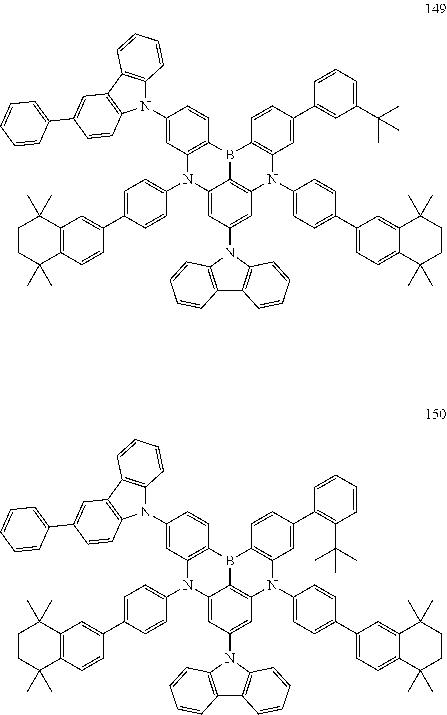 [SMILES: CC(C)(C)c1cccc(-c2ccc3c(c2)N(c2ccc(-c4ccc5c(c4)C(C)(C)CCC5(C)C)cc2)c2cc(-n4c5ccccc5c5ccccc54)cc4c2B3c2ccc(-n3c5ccccc5c5cc(-c6ccccc6)ccc53)cc2N4c2ccc(-c3ccc4c(c3)C(C)(C)CCC4(C)C)cc2)c1 ; CC(C)(C)c1ccccc1-c1ccc2c(c1)N(c1ccc(-c3ccc4c(c3)C(C)(C)CCC4(C)C)cc1)c1cc(-n3c4ccccc4c4ccccc43)cc3c1B2c1ccc(-n2c4ccccc4c4cc(-c5ccccc5)ccc42)cc1N3c1ccc(-c2ccc3c(c2)C(C)(C)CCC3(C)C)cc1]

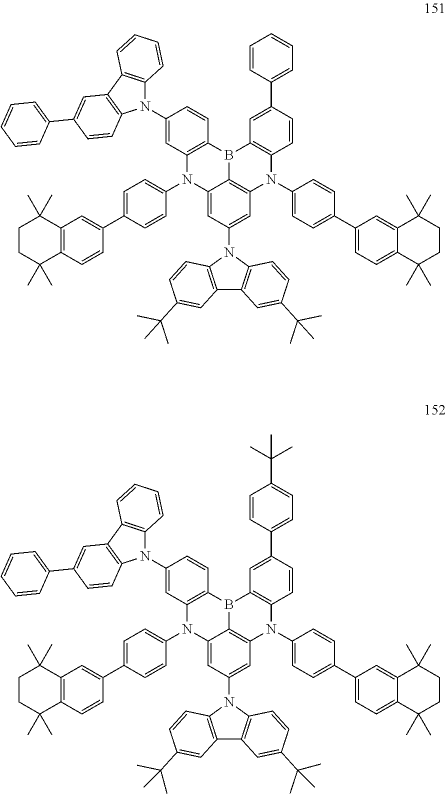 [SMILES: CC(C)(C)c1ccc2c(c1)c1cc(C(C)(C)C)ccc1n2-c1cc2c3c(c1)N(c1ccc(-c4ccc5c(c4)C(C)(C)CCC5(C)C)cc1)c1cc(-n4c5ccccc5c5cc(-c6ccccc6)ccc54)ccc1B3c1cc(-c3ccccc3)ccc1N2c1ccc(-c2ccc3c(c2)C(C)(C)CCC3(C)C)cc1 ; CC(C)(C)c1ccc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CCC4(C)C)cc2)cc1]

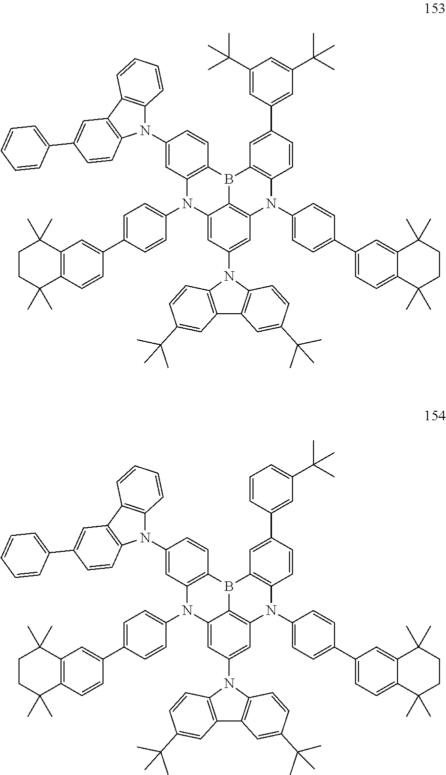 [SMILES: CC(C)(C)c1cc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CCC4(C)C)cc2)cc(C(C)(C)C)c1 ; CC(C)(C)c1cccc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CCC4(C)C)cc2)c1]

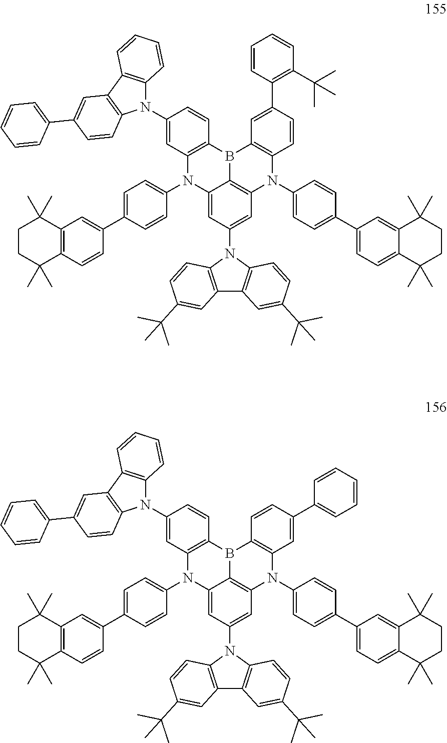 [SMILES: CC(C)(C)c1ccc2c(c1)c1cc(C(C)(C)C)ccc1n2-c1cc2c3c(c1)N(c1ccc(-c4ccc5c(c4)C(C)(C)CCC5(C)C)cc1)c1cc(-n4c5ccccc5c5cc(-c6ccccc6)ccc54)ccc1B3c1cc(-c3ccccc3C(C)(C)C)ccc1N2c1ccc(-c2ccc3c(c2)C(C)(C)CCC3(C)C)cc1 ; CC(C)(C)c1ccc2c(c1)c1cc(C(C)(C)C)ccc1n2-c1cc2c3c(c1)N(c1ccc(-c4ccc5c(c4)C(C)(C)CCC5(C)C)cc1)c1cc(-n4c5ccccc5c5cc(-c6ccccc6)ccc54)ccc1B3c1ccc(-c3ccccc3)cc1N2c1ccc(-c2ccc3c(c2)C(C)(C)CCC3(C)C)cc1]

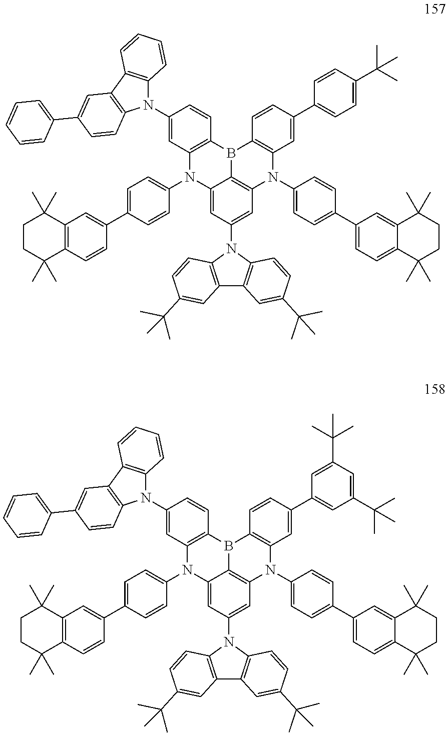

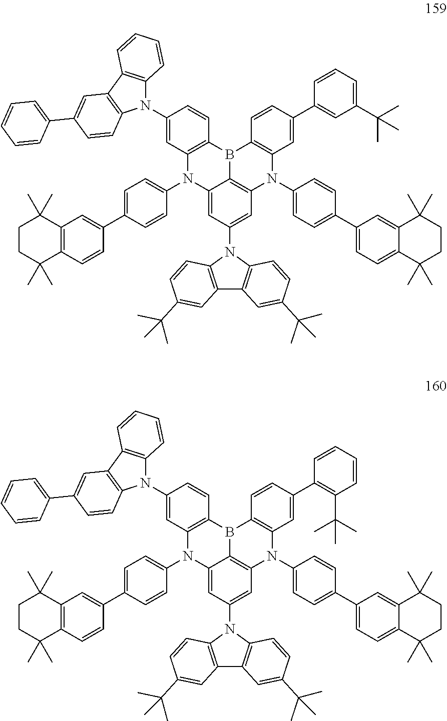 [SMILES: CC(C)(C)c1cccc(-c2ccc3c(c2)N(c2ccc(-c4ccc5c(c4)C(C)(C)CCC5(C)C)cc2)c2cc(-n4c5ccc(C(C)(C)C)cc5c5cc(C(C)(C)C)ccc54)cc4c2B3c2ccc(-n3c5ccccc5c5cc(-c6ccccc6)ccc53)cc2N4c2ccc(-c3ccc4c(c3)C(C)(C)CCC4(C)C)cc2)c1 ; CC(C)(C)c1ccc2c(c1)c1cc(C(C)(C)C)ccc1n2-c1cc2c3c(c1)N(c1ccc(-c4ccc5c(c4)C(C)(C)CCC5(C)C)cc1)c1cc(-n4c5ccccc5c5cc(-c6ccccc6)ccc54)ccc1B3c1ccc(-c3ccccc3C(C)(C)C)cc1N2c1ccc(-c2ccc3c(c2)C(C)(C)CCC3(C)C)cc1]

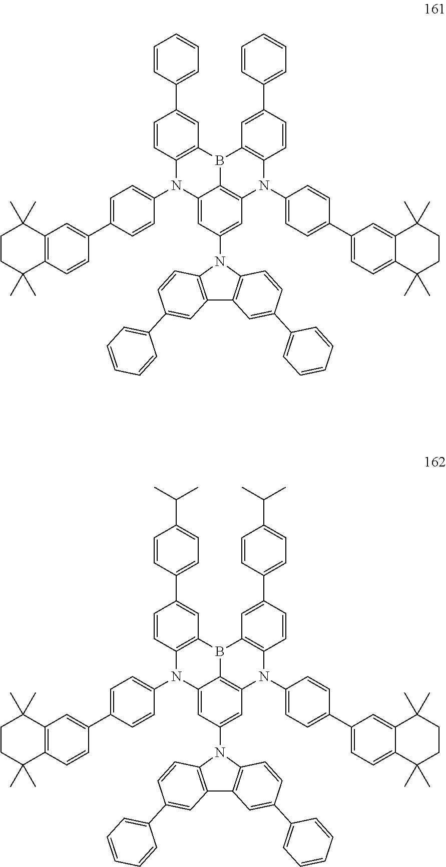 [SMILES: CC1(C)CCC(C)(C)c2cc(-c3ccc(N4c5ccc(-c6ccccc6)cc5B5c6cc(-c7ccccc7)ccc6N(c6ccc(-c7ccc8c(c7)C(C)(C)CCC8(C)C)cc6)c6cc(-n7c8ccc(-c9ccccc9)cc8c8cc(-c9ccccc9)ccc87)cc4c65)cc3)ccc21 ; CC(C)c1ccc(-c2ccc3c(c2)B2c4cc(-c5ccc(C(C)C)cc5)ccc4N(c4ccc(-c5ccc6c(c5)C(C)(C)CCC6(C)C)cc4)c4cc(-n5c6ccc(-c7ccccc7)cc6c6cc(-c7ccccc7)ccc65)cc(c42)N3c2ccc(-c3ccc4c(c3)C(C)(C)CCC4(C)C)cc2)cc1]

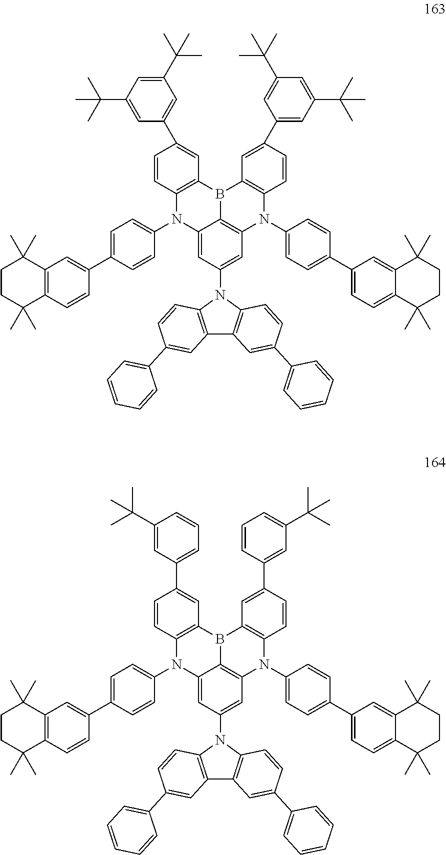 [SMILES: CC(C)(C)c1cc(-c2ccc3c(c2)B2c4cc(-c5cc(C(C)(C)C)cc(C(C)(C)C)c5)ccc4N(c4ccc(-c5ccc6c(c5)C(C)(C)CCC6(C)C)cc4)c4cc(-n5c6ccc(-c7ccccc7)cc6c6cc(-c7ccccc7)ccc65)cc(c42)N3c2ccc(-c3ccc4c(c3)C(C)(C)CCC4(C)C)cc2)cc(C(C)(C)C)c1 ; CC(C)(C)c1cccc(-c2ccc3c(c2)B2c4cc(-c5cccc(C(C)(C)C)c5)ccc4N(c4ccc(-c5ccc6c(c5)C(C)(C)CCC6(C)C)cc4)c4cc(-n5c6ccc(-c7ccccc7)cc6c6cc(-c7ccccc7)ccc65)cc(c42)N3c2ccc(-c3ccc4c(c3)C(C)(C)CCC4(C)C)cc2)c1]

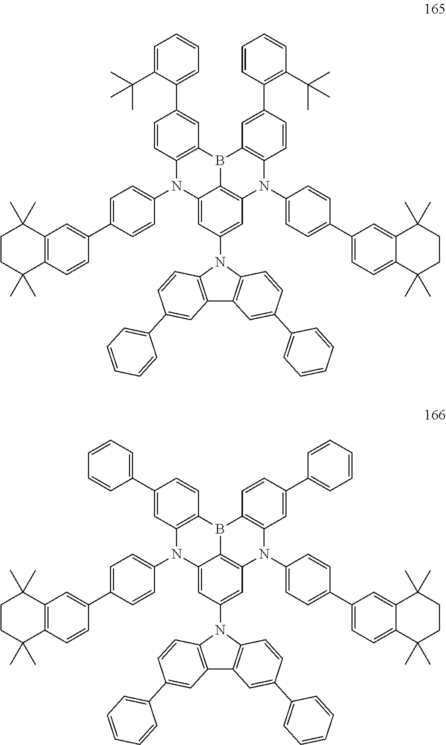 [SMILES: CC(C)(C)c1ccccc1-c1ccc2c(c1)B1c3cc(-c4ccccc4C(C)(C)C)ccc3N(c3ccc(-c4ccc5c(c4)C(C)(C)CCC5(C)C)cc3)c3cc(-n4c5ccc(-c6ccccc6)cc5c5cc(-c6ccccc6)ccc54)cc(c31)N2c1ccc(-c2ccc3c(c2)C(C)(C)CCC3(C)C)cc1 ; CC1(C)CCC(C)(C)c2cc(-c3ccc(N4c5cc(-c6ccccc6)ccc5B5c6ccc(-c7ccccc7)cc6N(c6ccc(-c7ccc8c(c7)C(C)(C)CCC8(C)C)cc6)c6cc(-n7c8ccc(-c9ccccc9)cc8c8cc(-c9ccccc9)ccc87)cc4c65)cc3)ccc21]

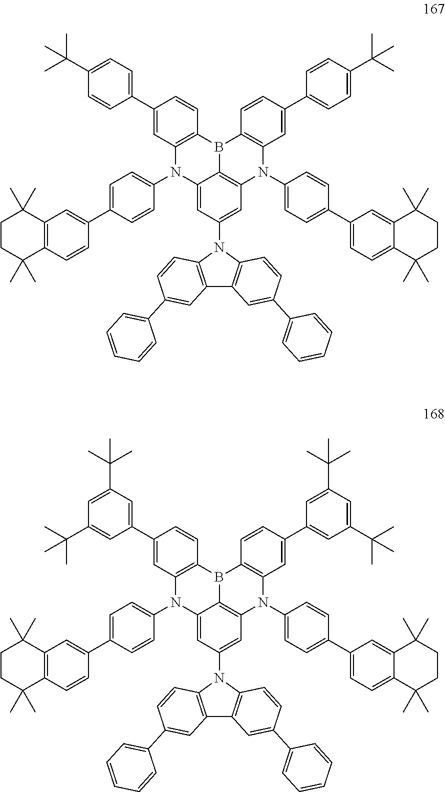 [SMILES: CC(C)(C)c1ccc(-c2ccc3c(c2)N(c2ccc(-c4ccc5c(c4)C(C)(C)CCC5(C)C)cc2)c2cc(-n4c5ccc(-c6ccccc6)cc5c5cc(-c6ccccc6)ccc54)cc4c2B3c2ccc(-c3ccc(C(C)(C)C)cc3)cc2N4c2ccc(-c3ccc4c(c3)C(C)(C)CCC4(C)C)cc2)cc1 ; CC(C)(C)c1cc(-c2ccc3c(c2)N(c2ccc(-c4ccc5c(c4)C(C)(C)CCC5(C)C)cc2)c2cc(-n4c5ccc(-c6ccccc6)cc5c5cc(-c6ccccc6)ccc54)cc4c2B3c2ccc(-c3cc(C(C)(C)C)cc(C(C)(C)C)c3)cc2N4c2ccc(-c3ccc4c(c3)C(C)(C)CCC4(C)C)cc2)cc(C(C)(C)C)c1]

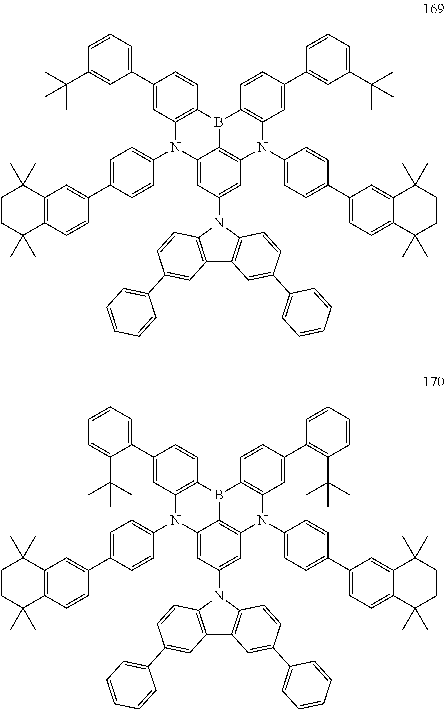 [SMILES: CC(C)(C)c1cccc(-c2ccc3c(c2)N(c2ccc(-c4ccc5c(c4)C(C)(C)CCC5(C)C)cc2)c2cc(-n4c5ccc(-c6ccccc6)cc5c5cc(-c6ccccc6)ccc54)cc4c2B3c2ccc(-c3cccc(C(C)(C)C)c3)cc2N4c2ccc(-c3ccc4c(c3)C(C)(C)CCC4(C)C)cc2)c1 ; CC(C)(C)c1ccccc1-c1ccc2c(c1)N(c1ccc(-c3ccc4c(c3)C(C)(C)CCC4(C)C)cc1)c1cc(-n3c4ccc(-c5ccccc5)cc4c4cc(-c5ccccc5)ccc43)cc3c1B2c1ccc(-c2ccccc2C(C)(C)C)cc1N3c1ccc(-c2ccc3c(c2)C(C)(C)CCC3(C)C)cc1]

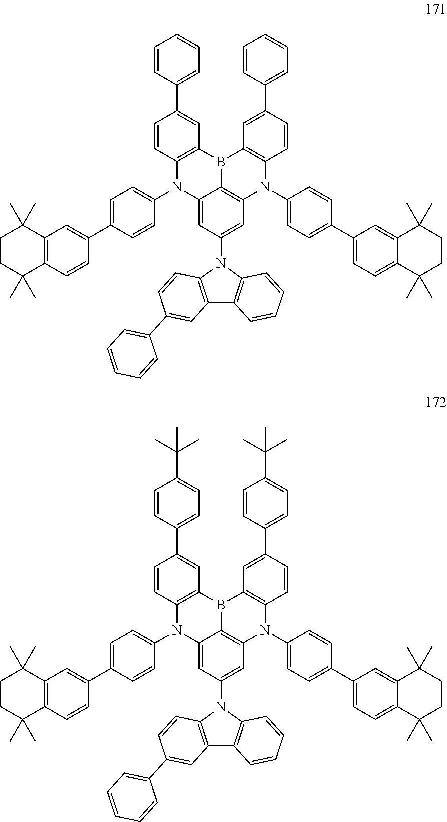 [SMILES: CC1(C)CCC(C)(C)c2cc(-c3ccc(N4c5ccc(-c6ccccc6)cc5B5c6cc(-c7ccccc7)ccc6N(c6ccc(-c7ccc8c(c7)C(C)(C)CCC8(C)C)cc6)c6cc(-n7c8ccccc8c8cc(-c9ccccc9)ccc87)cc4c65)cc3)ccc21 ; CC(C)(C)c1ccc(-c2ccc3c(c2)B2c4cc(-c5ccc(C(C)(C)C)cc5)ccc4N(c4ccc(-c5ccc6c(c5)C(C)(C)CCC6(C)C)cc4)c4cc(-n5c6ccccc6c6cc(-c7ccccc7)ccc65)cc(c42)N3c2ccc(-c3ccc4c(c3)C(C)(C)CCC4(C)C)cc2)cc1]

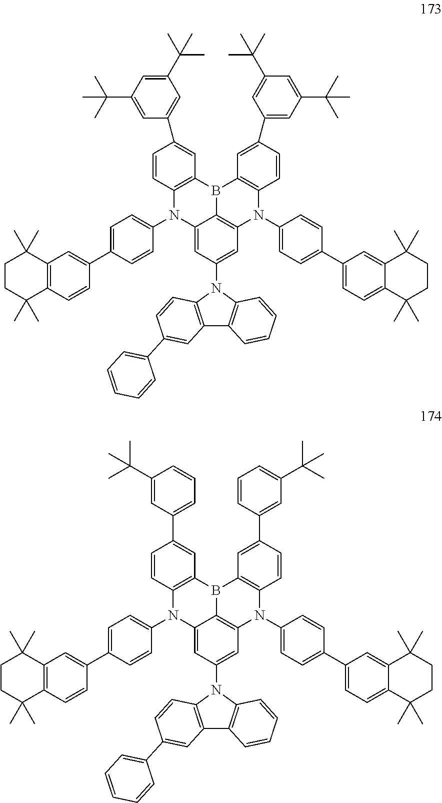 [SMILES: CC(C)(C)c1cc(-c2ccc3c(c2)B2c4cc(-c5cc(C(C)(C)C)cc(C(C)(C)C)c5)ccc4N(c4ccc(-c5ccc6c(c5)C(C)(C)CCC6(C)C)cc4)c4cc(-n5c6ccccc6c6cc(-c7ccccc7)ccc65)cc(c42)N3c2ccc(-c3ccc4c(c3)C(C)(C)CCC4(C)C)cc2)cc(C(C)(C)C)c1 ; CC(C)(C)c1cccc(-c2ccc3c(c2)B2c4cc(-c5cccc(C(C)(C)C)c5)ccc4N(c4ccc(-c5ccc6c(c5)C(C)(C)CCC6(C)C)cc4)c4cc(-n5c6ccccc6c6cc(-c7ccccc7)ccc65)cc(c42)N3c2ccc(-c3ccc4c(c3)C(C)(C)CCC4(C)C)cc2)c1]

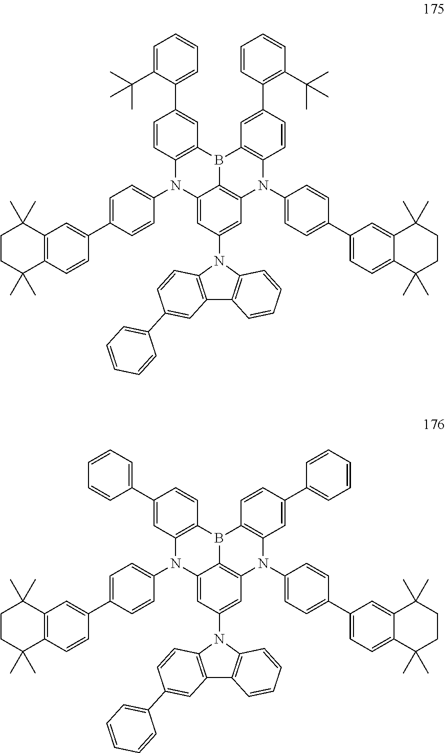 [SMILES: CC(C)(C)c1ccccc1-c1ccc2c(c1)B1c3cc(-c4ccccc4C(C)(C)C)ccc3N(c3ccc(-c4ccc5c(c4)C(C)(C)CCC5(C)C)cc3)c3cc(-n4c5ccccc5c5cc(-c6ccccc6)ccc54)cc(c31)N2c1ccc(-c2ccc3c(c2)C(C)(C)CCC3(C)C)cc1 ; CC1(C)CCC(C)(C)c2cc(-c3ccc(N4c5cc(-c6ccccc6)ccc5B5c6ccc(-c7ccccc7)cc6N(c6ccc(-c7ccc8c(c7)C(C)(C)CCC8(C)C)cc6)c6cc(-n7c8ccccc8c8cc(-c9ccccc9)ccc87)cc4c65)cc3)ccc21]

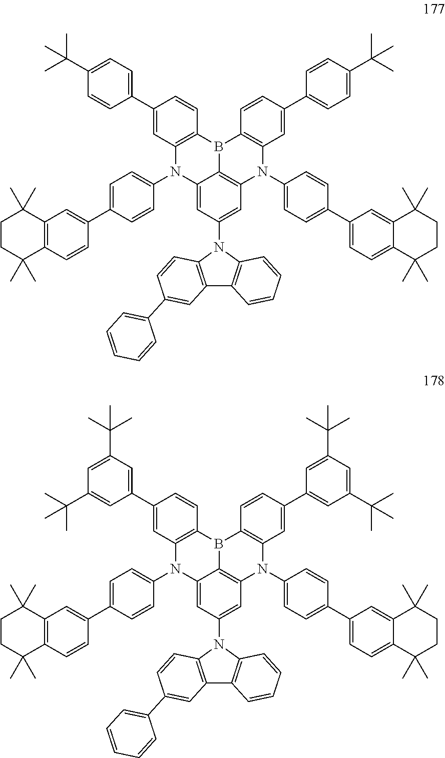 [SMILES: CC(C)(C)c1ccc(-c2ccc3c(c2)N(c2ccc(-c4ccc5c(c4)C(C)(C)CCC5(C)C)cc2)c2cc(-n4c5ccccc5c5cc(-c6ccccc6)ccc54)cc4c2B3c2ccc(-c3ccc(C(C)(C)C)cc3)cc2N4c2ccc(-c3ccc4c(c3)C(C)(C)CCC4(C)C)cc2)cc1 ; CC(C)(C)c1cc(-c2ccc3c(c2)N(c2ccc(-c4ccc5c(c4)C(C)(C)CCC5(C)C)cc2)c2cc(-n4c5ccccc5c5cc(-c6ccccc6)ccc54)cc4c2B3c2ccc(-c3cc(C(C)(C)C)cc(C(C)(C)C)c3)cc2N4c2ccc(-c3ccc4c(c3)C(C)(C)CCC4(C)C)cc2)cc(C(C)(C)C)c1]

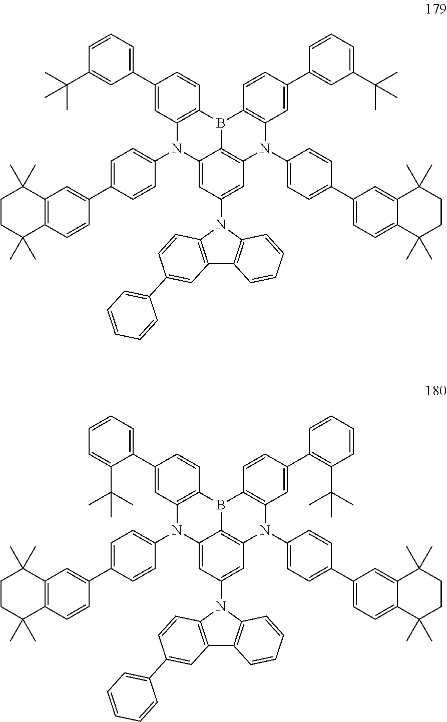 [SMILES: CC(C)(C)c1cccc(-c2ccc3c(c2)N(c2ccc(-c4ccc5c(c4)C(C)(C)CCC5(C)C)cc2)c2cc(-n4c5ccccc5c5cc(-c6ccccc6)ccc54)cc4c2B3c2ccc(-c3cccc(C(C)(C)C)c3)cc2N4c2ccc(-c3ccc4c(c3)C(C)(C)CCC4(C)C)cc2)c1 ; CC(C)(C)c1ccccc1-c1ccc2c(c1)N(c1ccc(-c3ccc4c(c3)C(C)(C)CCC4(C)C)cc1)c1cc(-n3c4ccccc4c4cc(-c5ccccc5)ccc43)cc3c1B2c1ccc(-c2ccccc2C(C)(C)C)cc1N3c1ccc(-c2ccc3c(c2)C(C)(C)CCC3(C)C)cc1]

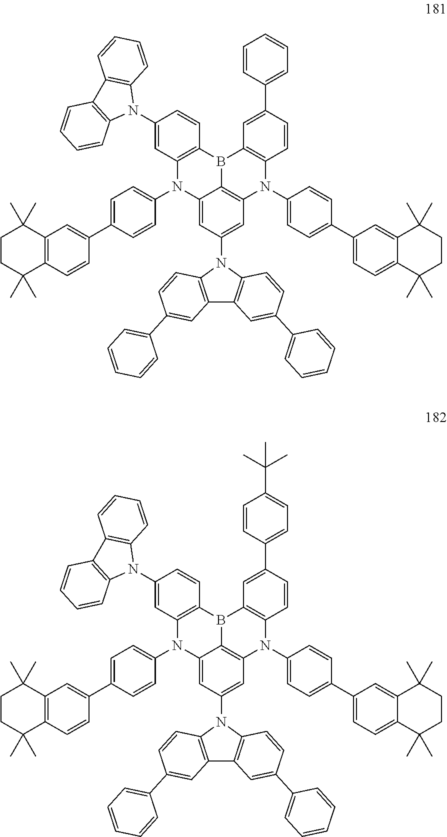 [SMILES: CC1(C)CCC(C)(C)c2cc(-c3ccc(N4c5ccc(-c6ccccc6)cc5B5c6ccc(-n7c8ccccc8c8ccccc87)cc6N(c6ccc(-c7ccc8c(c7)C(C)(C)CCC8(C)C)cc6)c6cc(-n7c8ccc(-c9ccccc9)cc8c8cc(-c9ccccc9)ccc87)cc4c65)cc3)ccc21 ; CC(C)(C)c1ccc(-c2ccc3c(c2)B2c4ccc(-n5c6ccccc6c6ccccc65)cc4N(c4ccc(-c5ccc6c(c5)C(C)(C)CCC6(C)C)cc4)c4cc(-n5c6ccc(-c7ccccc7)cc6c6cc(-c7ccccc7)ccc65)cc(c42)N3c2ccc(-c3ccc4c(c3)C(C)(C)CCC4(C)C)cc2)cc1]

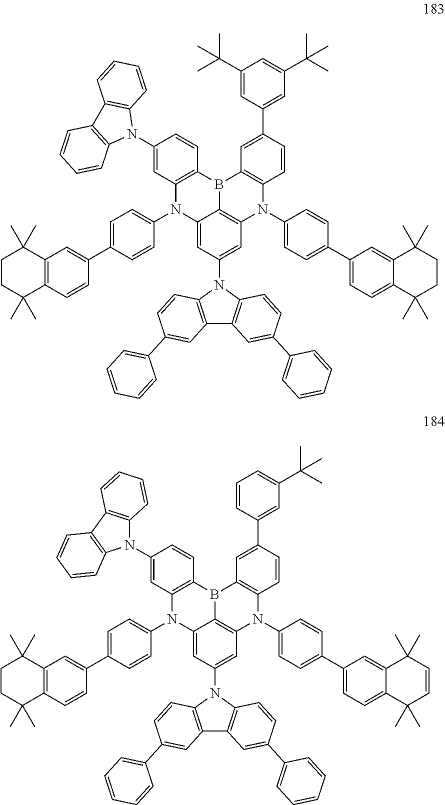

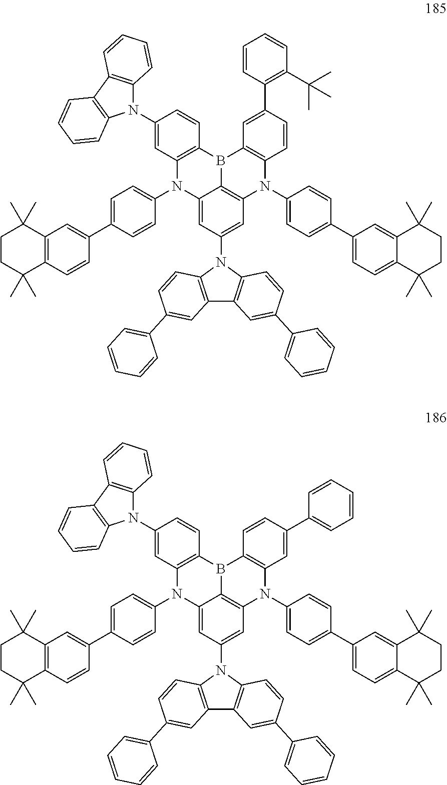 [SMILES: CC(C)(C)c1ccccc1-c1ccc2c(c1)B1c3ccc(-n4c5ccccc5c5ccccc54)cc3N(c3ccc(-c4ccc5c(c4)C(C)(C)CCC5(C)C)cc3)c3cc(-n4c5ccc(-c6ccccc6)cc5c5cc(-c6ccccc6)ccc54)cc(c31)N2c1ccc(-c2ccc3c(c2)C(C)(C)CCC3(C)C)cc1 ; CC1(C)CCC(C)(C)c2cc(-c3ccc(N4c5cc(-c6ccccc6)ccc5B5c6ccc(-n7c8ccccc8c8ccccc87)cc6N(c6ccc(-c7ccc8c(c7)C(C)(C)CCC8(C)C)cc6)c6cc(-n7c8ccc(-c9ccccc9)cc8c8cc(-c9ccccc9)ccc87)cc4c65)cc3)ccc21]

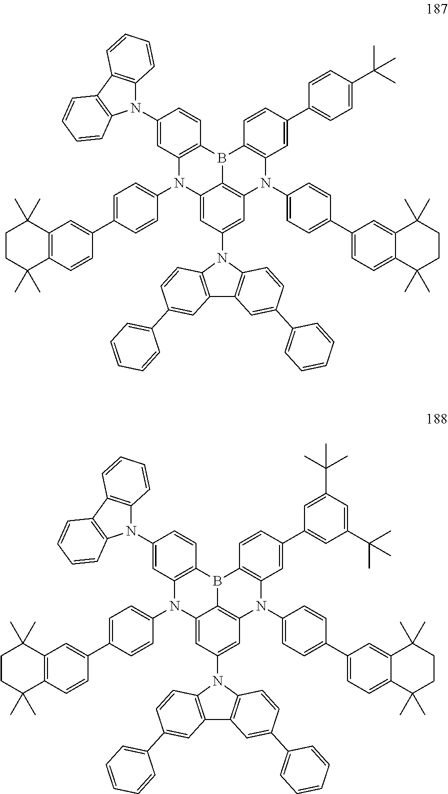

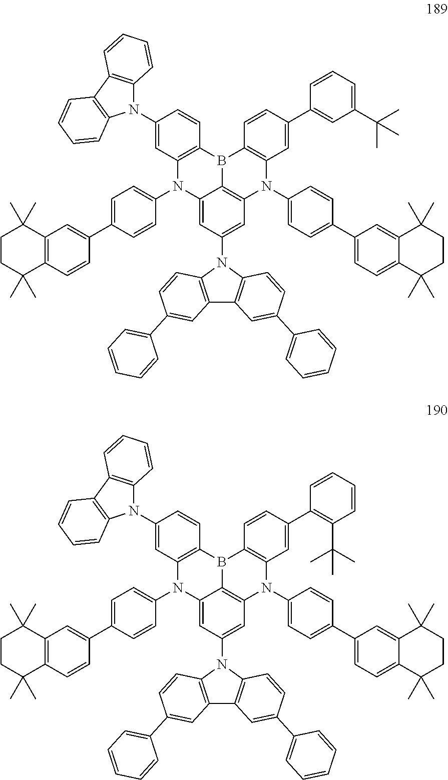 [SMILES: CC(C)(C)c1cccc(-c2ccc3c(c2)N(c2ccc(-c4ccc5c(c4)C(C)(C)CCC5(C)C)cc2)c2cc(-n4c5ccc(-c6ccccc6)cc5c5cc(-c6ccccc6)ccc54)cc4c2B3c2ccc(-n3c5ccccc5c5ccccc53)cc2N4c2ccc(-c3ccc4c(c3)C(C)(C)CCC4(C)C)cc2)c1 ; CC(C)(C)c1ccccc1-c1ccc2c(c1)N(c1ccc(-c3ccc4c(c3)C(C)(C)CCC4(C)C)cc1)c1cc(-n3c4ccc(-c5ccccc5)cc4c4cc(-c5ccccc5)ccc43)cc3c1B2c1ccc(-n2c4ccccc4c4ccccc42)cc1N3c1ccc(-c2ccc3c(c2)C(C)(C)CCC3(C)C)cc1]

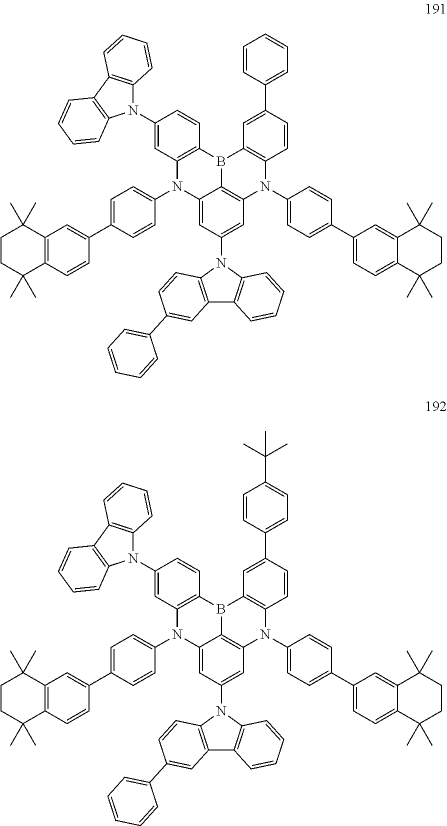 [SMILES: CC1(C)CCC(C)(C)c2cc(-c3ccc(N4c5ccc(-c6ccccc6)cc5B5c6ccc(-n7c8ccccc8c8ccccc87)cc6N(c6ccc(-c7ccc8c(c7)C(C)(C)CCC8(C)C)cc6)c6cc(-n7c8ccccc8c8cc(-c9ccccc9)ccc87)cc4c65)cc3)ccc21 ; CC(C)(C)c1ccc(-c2ccc3c(c2)B2c4ccc(-n5c6ccccc6c6ccccc65)cc4N(c4ccc(-c5ccc6c(c5)C(C)(C)CCC6(C)C)cc4)c4cc(-n5c6ccccc6c6cc(-c7ccccc7)ccc65)cc(c42)N3c2ccc(-c3ccc4c(c3)C(C)(C)CCC4(C)C)cc2)cc1]

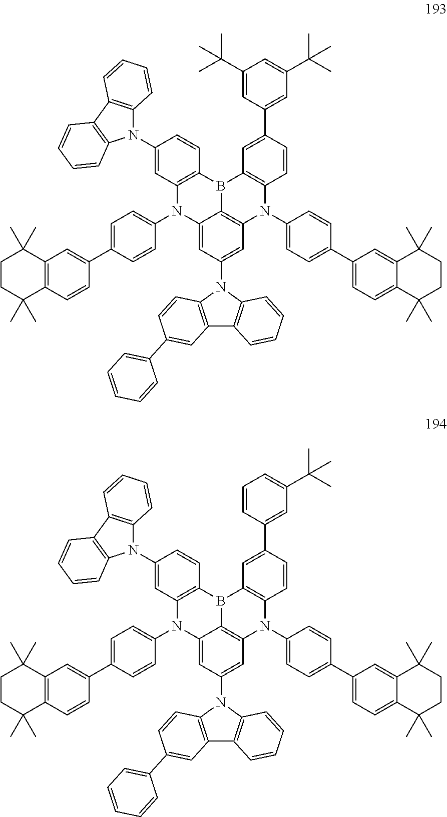 [SMILES: CC(C)(C)c1cc(-c2ccc3c(c2)B2c4ccc(-n5c6ccccc6c6ccccc65)cc4N(c4ccc(-c5ccc6c(c5)C(C)(C)CCC6(C)C)cc4)c4cc(-n5c6ccccc6c6cc(-c7ccccc7)ccc65)cc(c42)N3c2ccc(-c3ccc4c(c3)C(C)(C)CCC4(C)C)cc2)cc(C(C)(C)C)c1 ; CC(C)(C)c1cccc(-c2ccc3c(c2)B2c4ccc(-n5c6ccccc6c6ccccc65)cc4N(c4ccc(-c5ccc6c(c5)C(C)(C)CCC6(C)C)cc4)c4cc(-n5c6ccccc6c6cc(-c7ccccc7)ccc65)cc(c42)N3c2ccc(-c3ccc4c(c3)C(C)(C)CCC4(C)C)cc2)c1]

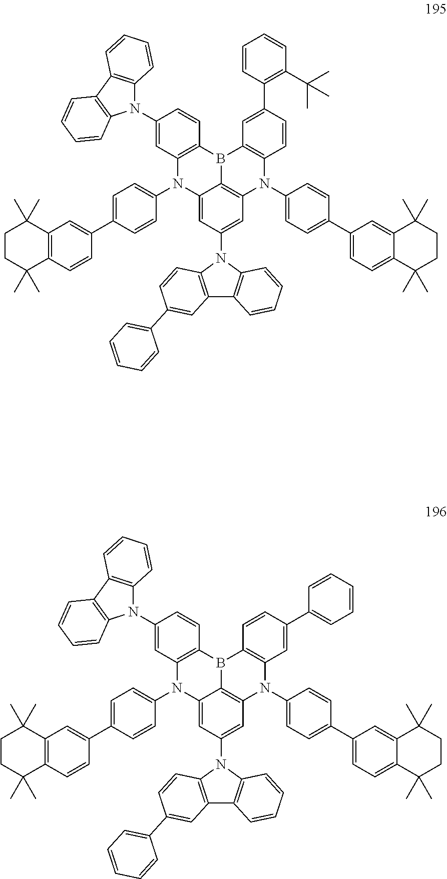 [SMILES: CC(C)(C)c1ccccc1-c1ccc2c(c1)B1c3ccc(-n4c5ccccc5c5ccccc54)cc3N(c3ccc(-c4ccc5c(c4)C(C)(C)CCC5(C)C)cc3)c3cc(-n4c5ccccc5c5cc(-c6ccccc6)ccc54)cc(c31)N2c1ccc(-c2ccc3c(c2)C(C)(C)CCC3(C)C)cc1 ; CC1(C)CCC(C)(C)c2cc(-c3ccc(N4c5cc(-c6ccccc6)ccc5B5c6ccc(-n7c8ccccc8c8ccccc87)cc6N(c6ccc(-c7ccc8c(c7)C(C)(C)CCC8(C)C)cc6)c6cc(-n7c8ccccc8c8cc(-c9ccccc9)ccc87)cc4c65)cc3)ccc21]

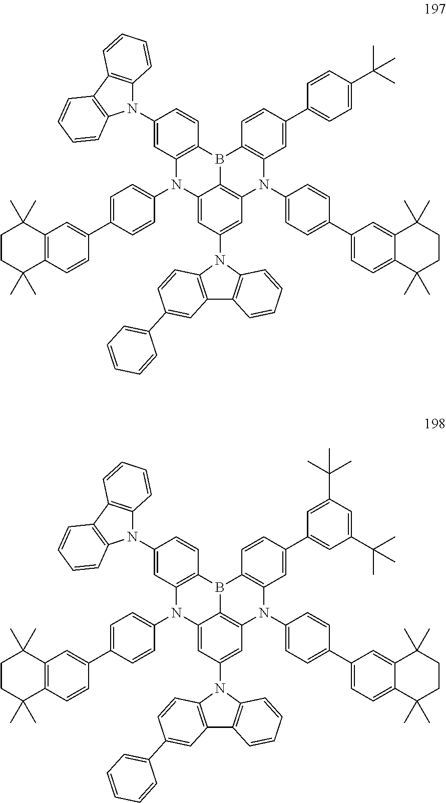 [SMILES: CC(C)(C)c1ccc(-c2ccc3c(c2)N(c2ccc(-c4ccc5c(c4)C(C)(C)CCC5(C)C)cc2)c2cc(-n4c5ccccc5c5cc(-c6ccccc6)ccc54)cc4c2B3c2ccc(-n3c5ccccc5c5ccccc53)cc2N4c2ccc(-c3ccc4c(c3)C(C)(C)CCC4(C)C)cc2)cc1 ; CC(C)(C)c1cc(-c2ccc3c(c2)N(c2ccc(-c4ccc5c(c4)C(C)(C)CCC5(C)C)cc2)c2cc(-n4c5ccccc5c5cc(-c6ccccc6)ccc54)cc4c2B3c2ccc(-n3c5ccccc5c5ccccc53)cc2N4c2ccc(-c3ccc4c(c3)C(C)(C)CCC4(C)C)cc2)cc(C(C)(C)C)c1]

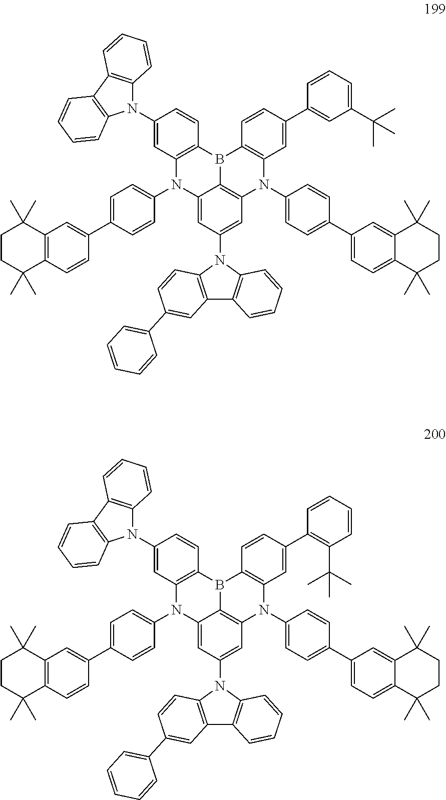 [SMILES: CC(C)(C)c1cccc(-c2ccc3c(c2)N(c2ccc(-c4ccc5c(c4)C(C)(C)CCC5(C)C)cc2)c2cc(-n4c5ccccc5c5cc(-c6ccccc6)ccc54)cc4c2B3c2ccc(-n3c5ccccc5c5ccccc53)cc2N4c2ccc(-c3ccc4c(c3)C(C)(C)CCC4(C)C)cc2)c1 ; CC(C)(C)c1ccccc1-c1ccc2c(c1)N(c1ccc(-c3ccc4c(c3)C(C)(C)CCC4(C)C)cc1)c1cc(-n3c4ccccc4c4cc(-c5ccccc5)ccc43)cc3c1B2c1ccc(-n2c4ccccc4c4ccccc42)cc1N3c1ccc(-c2ccc3c(c2)C(C)(C)CCC3(C)C)cc1]

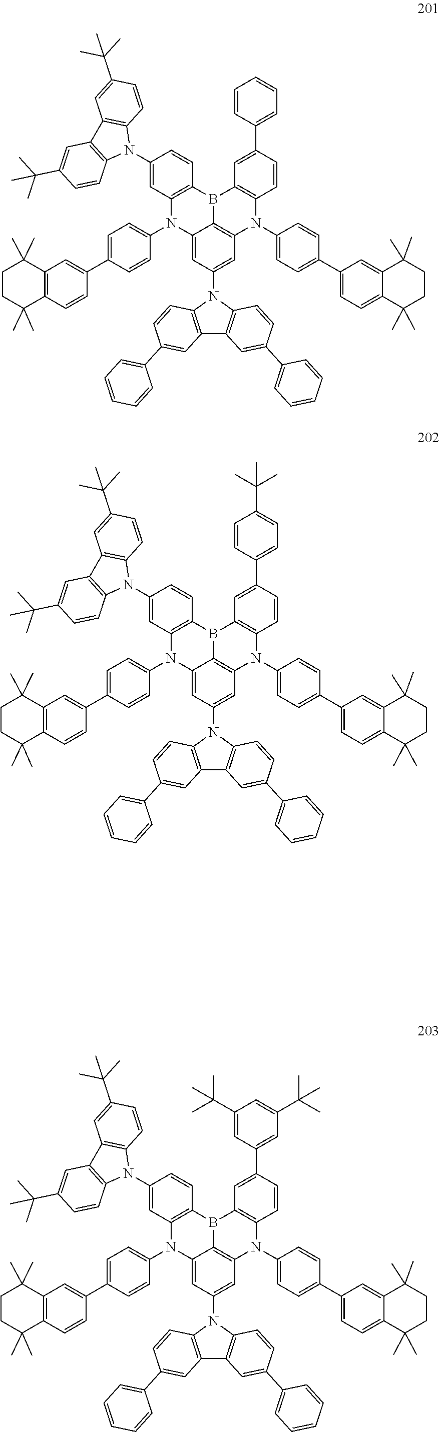

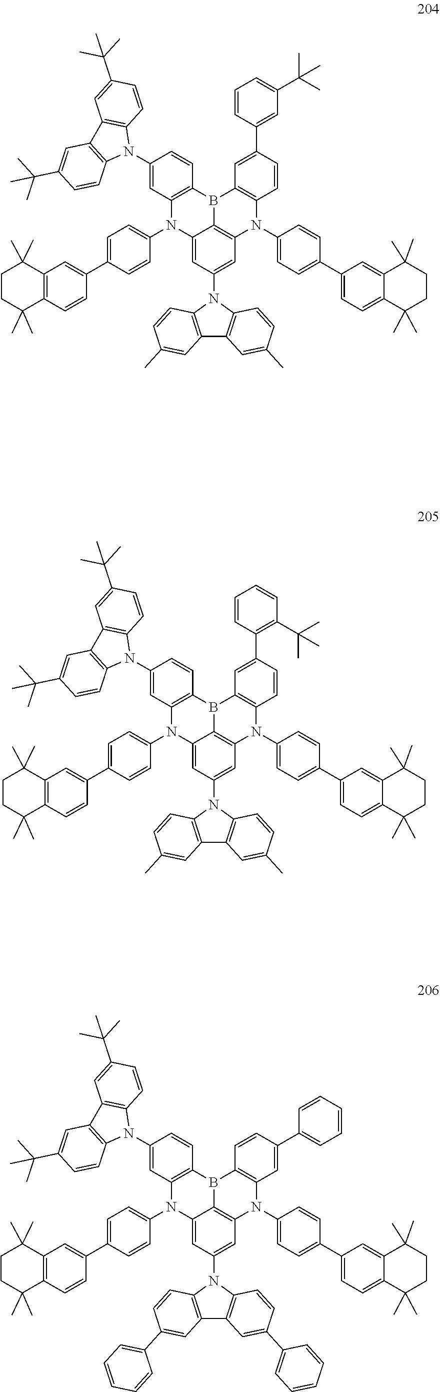

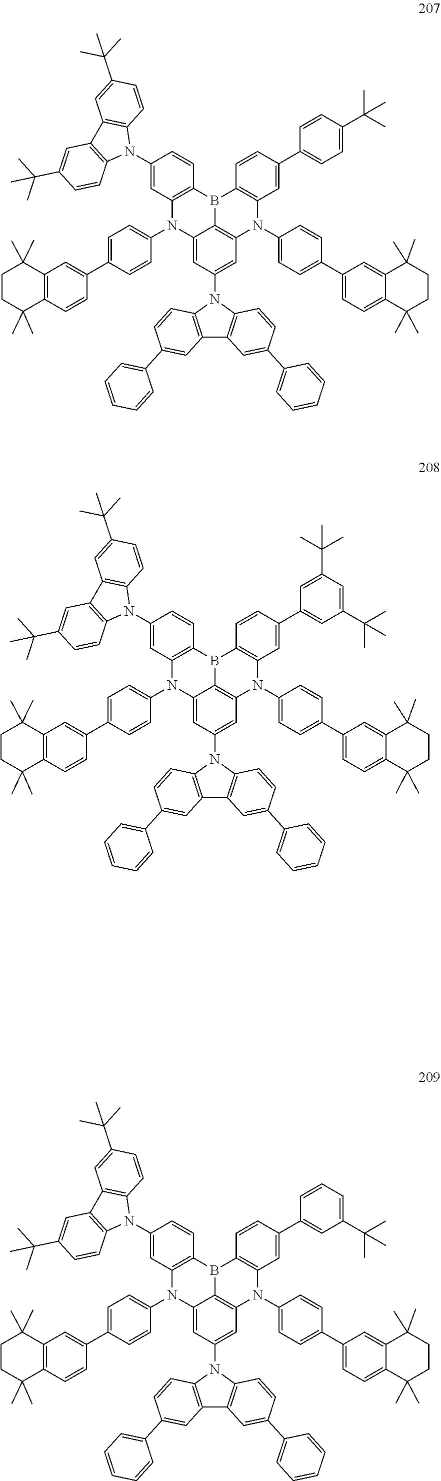

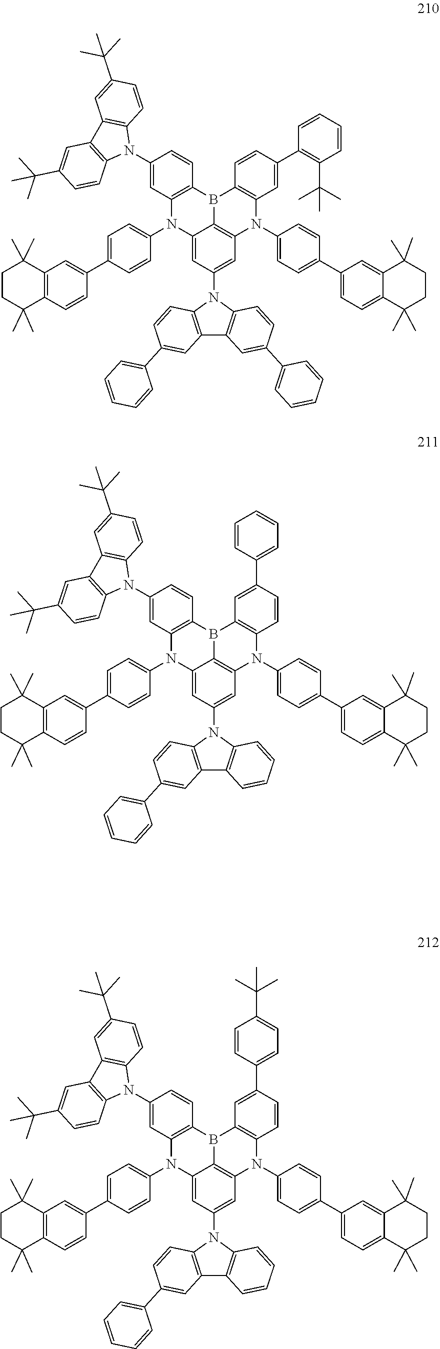

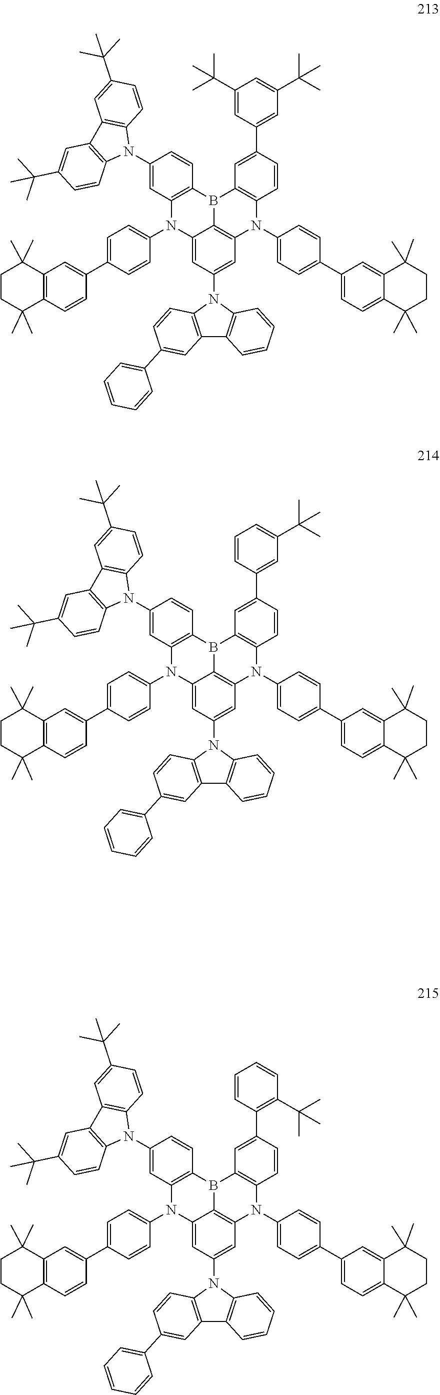

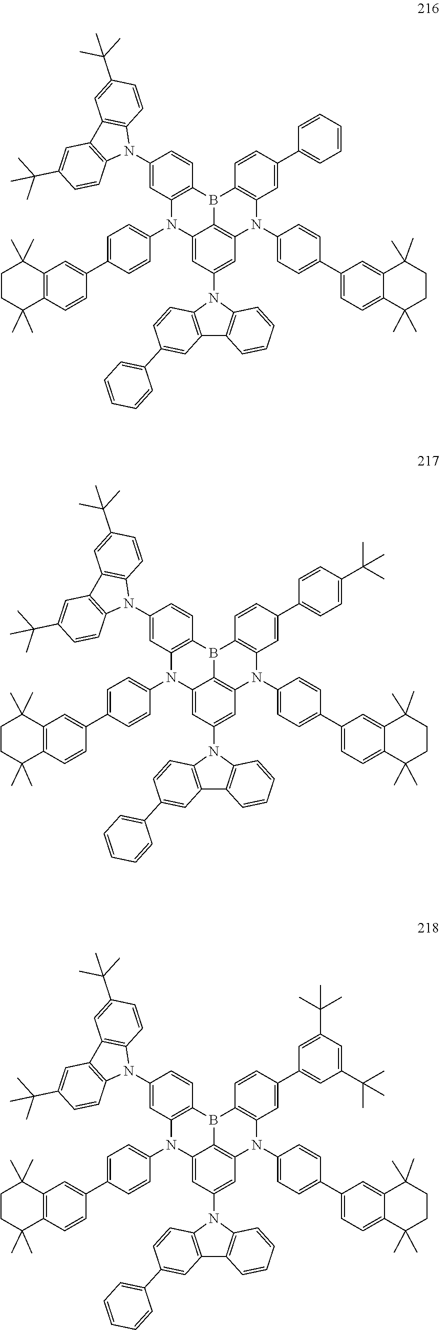

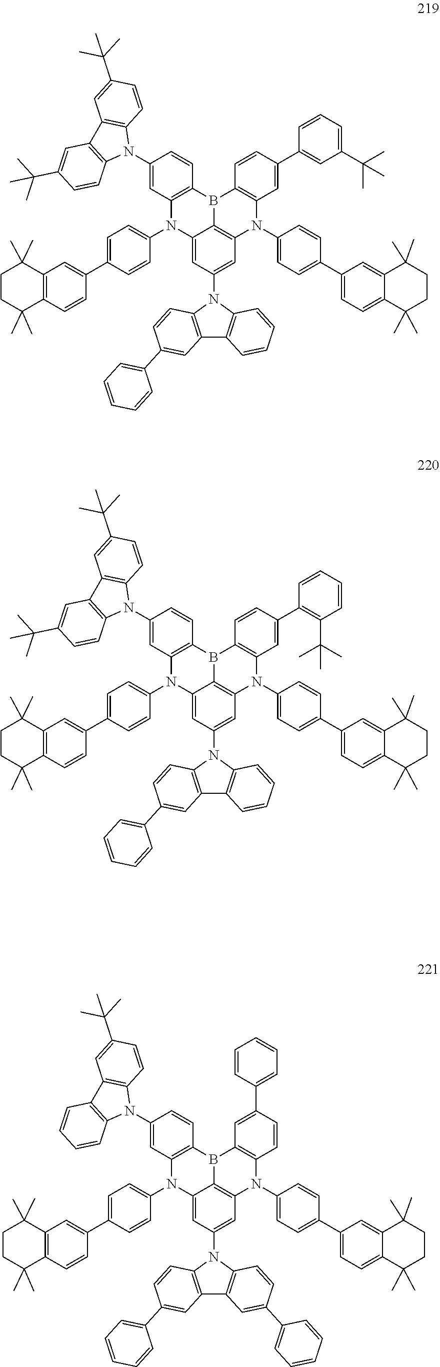

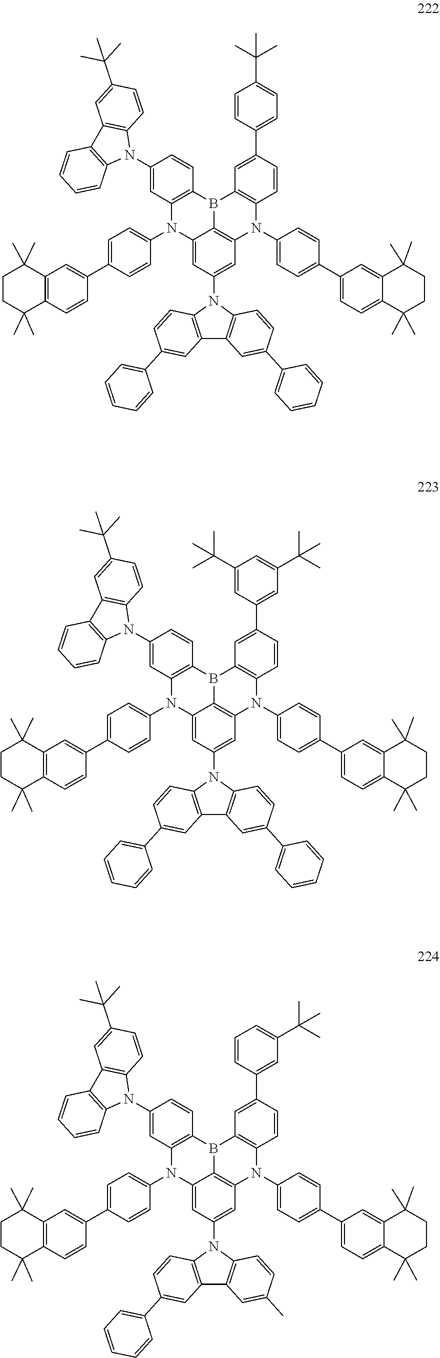

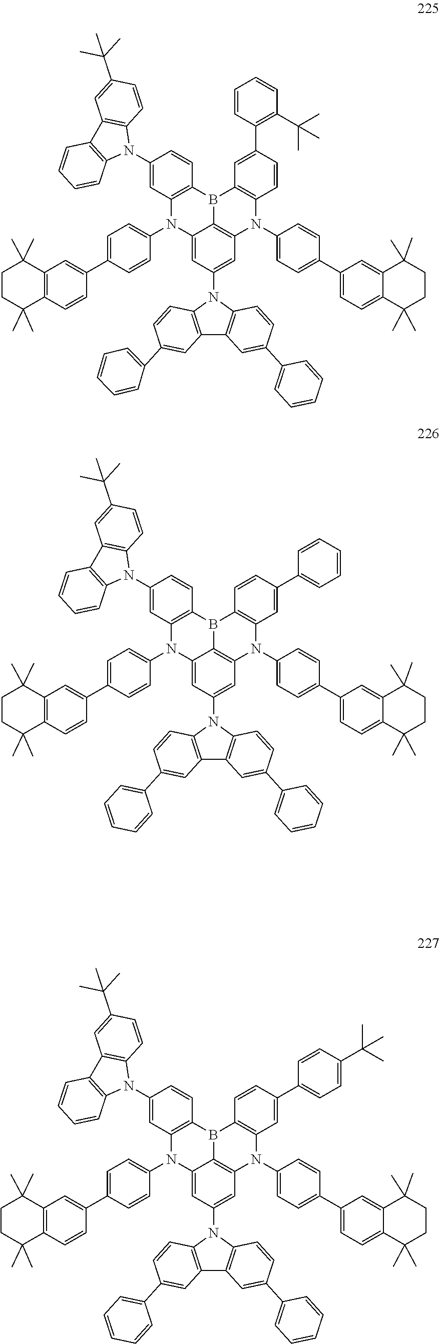

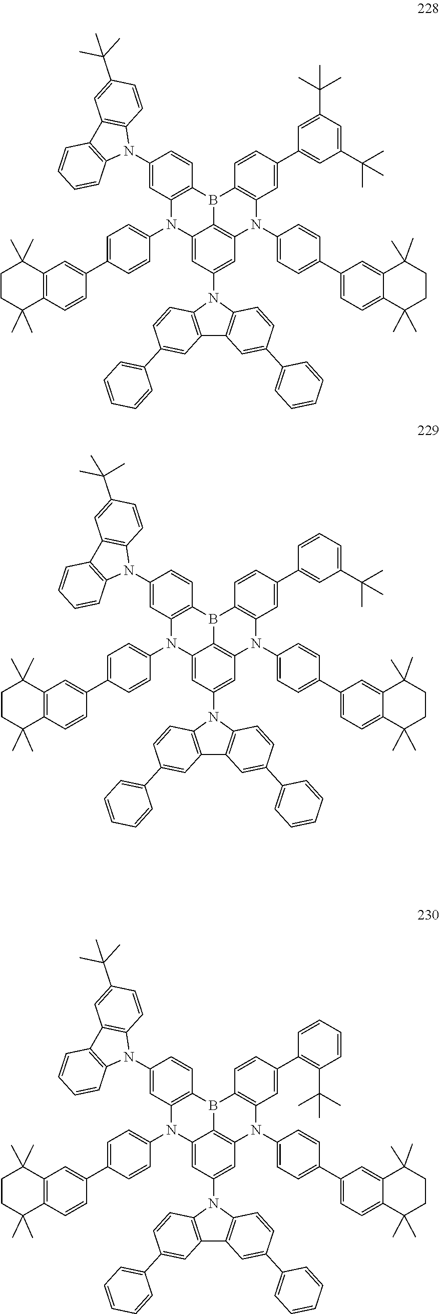

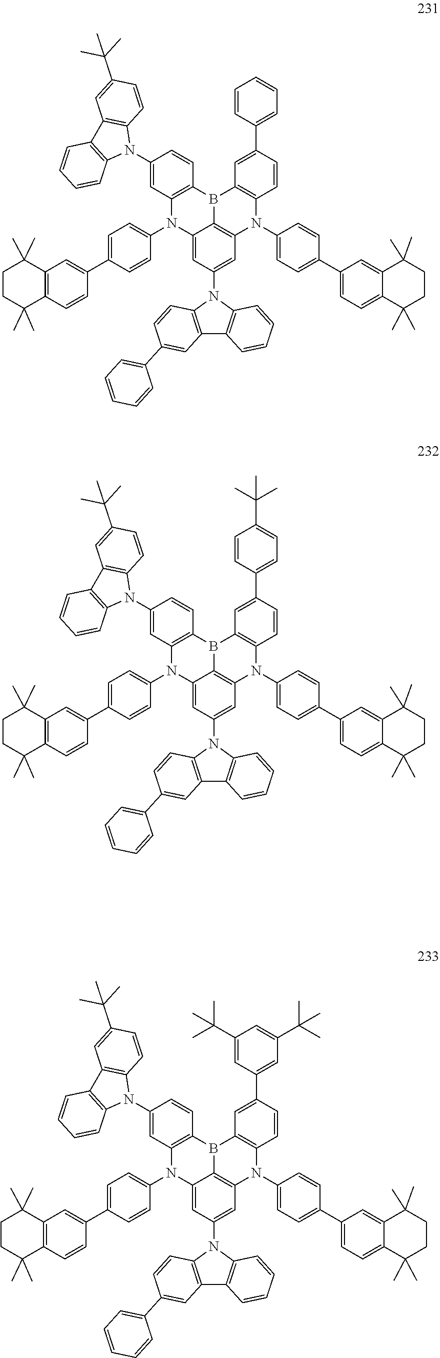

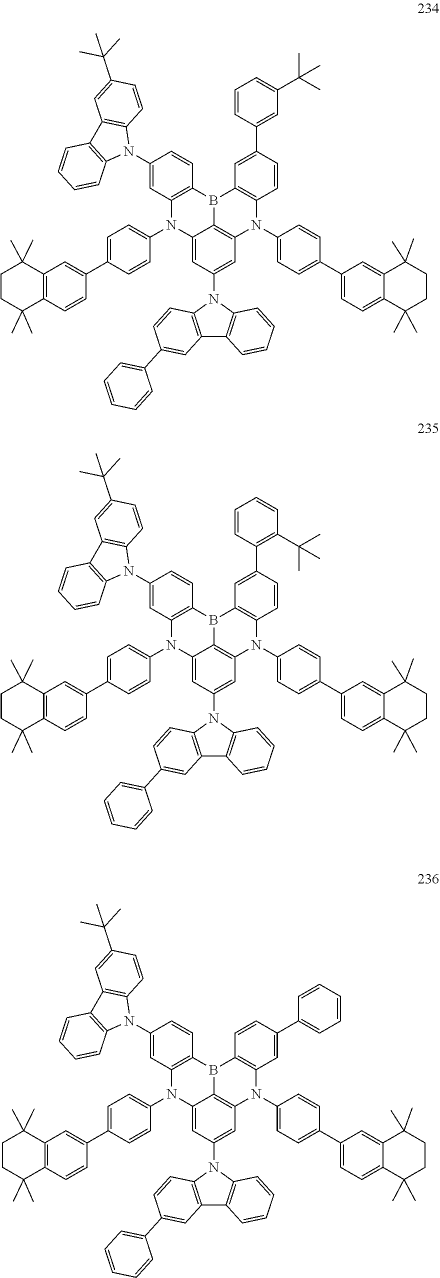

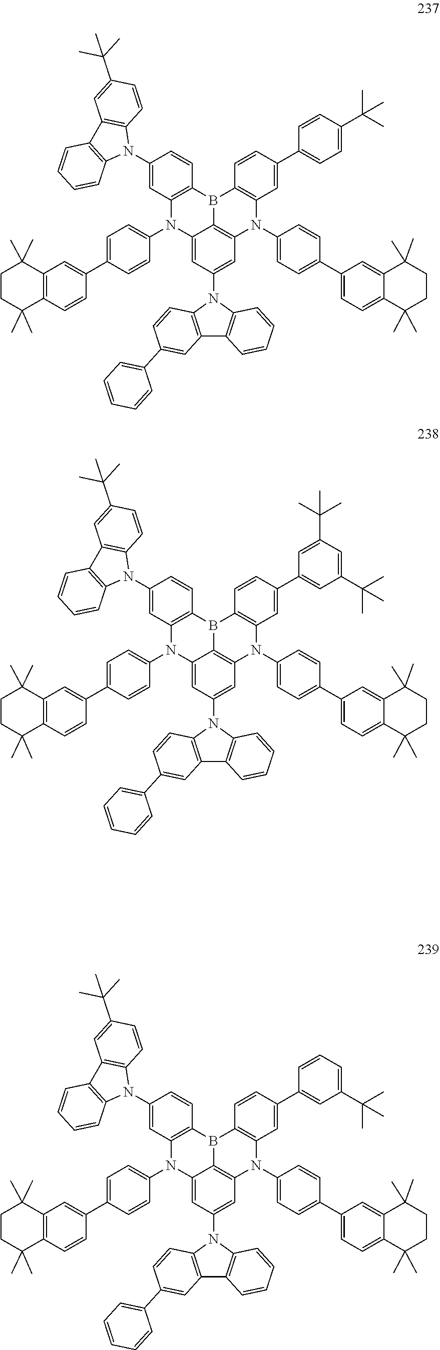

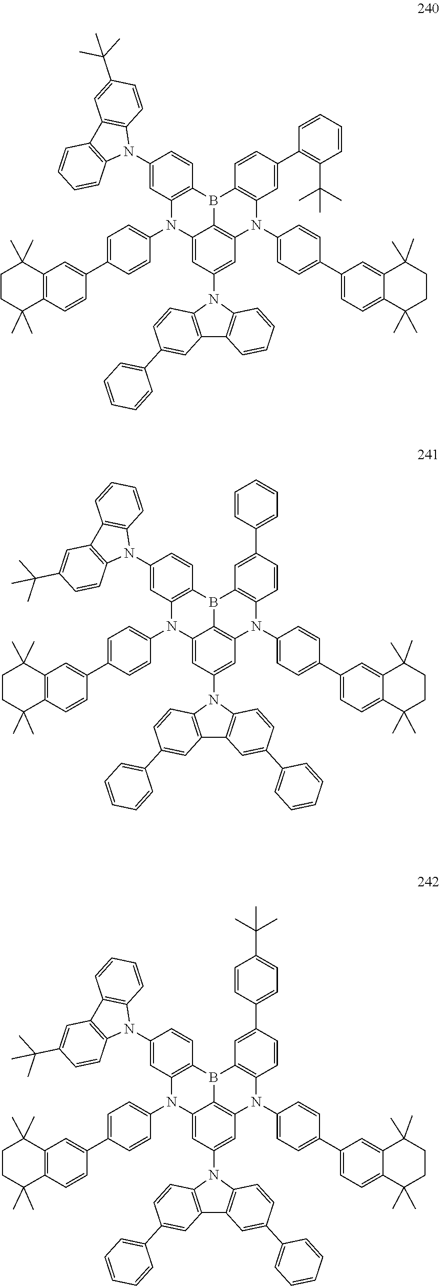

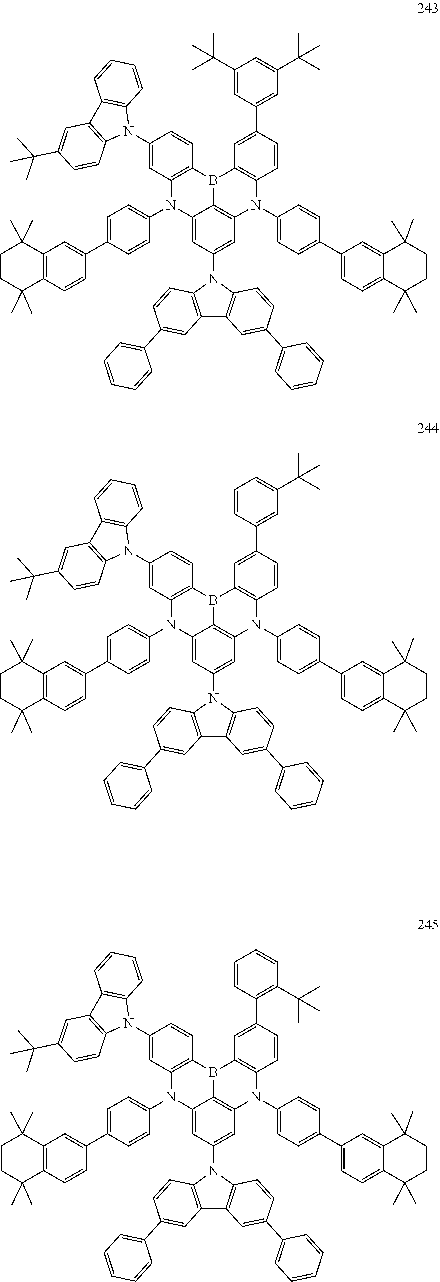

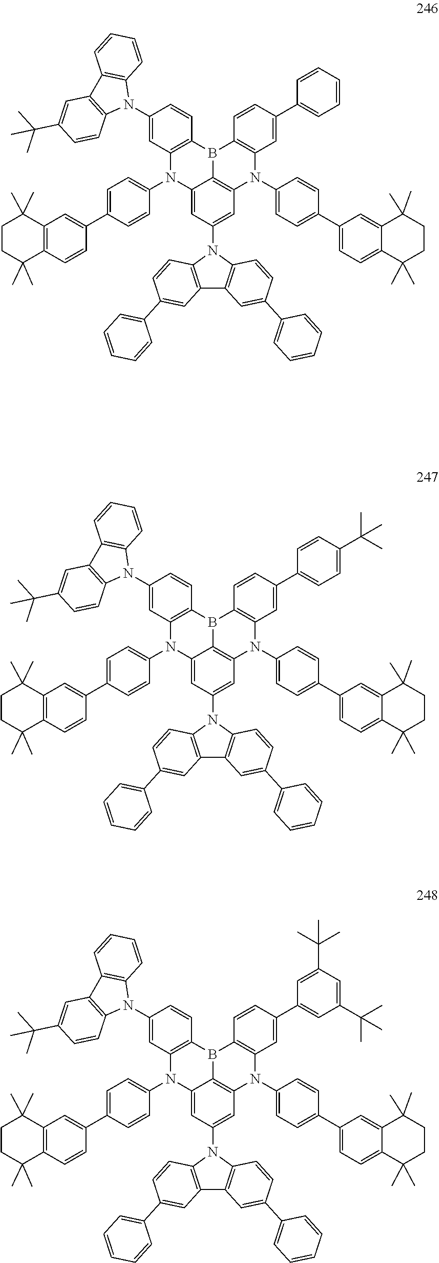

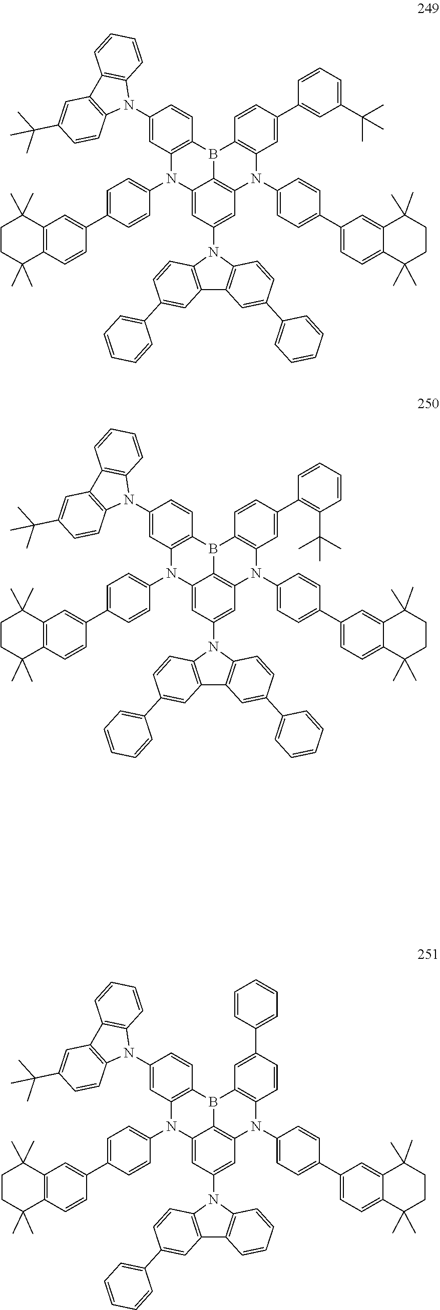

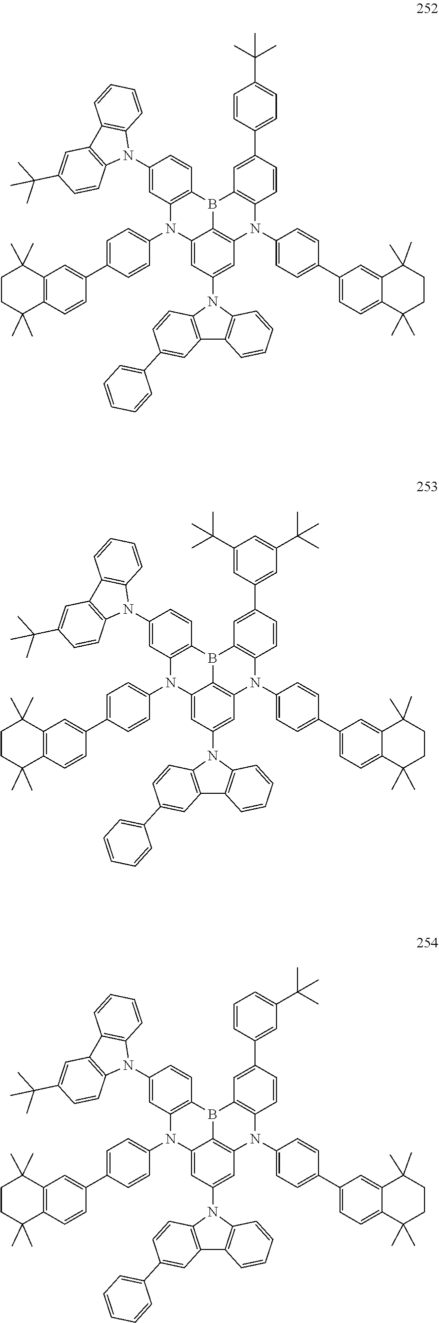

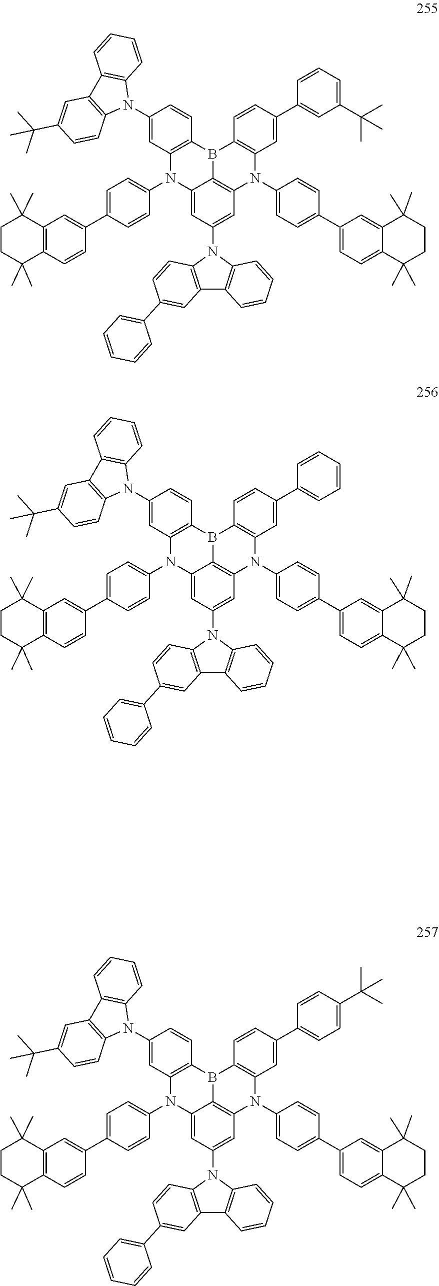

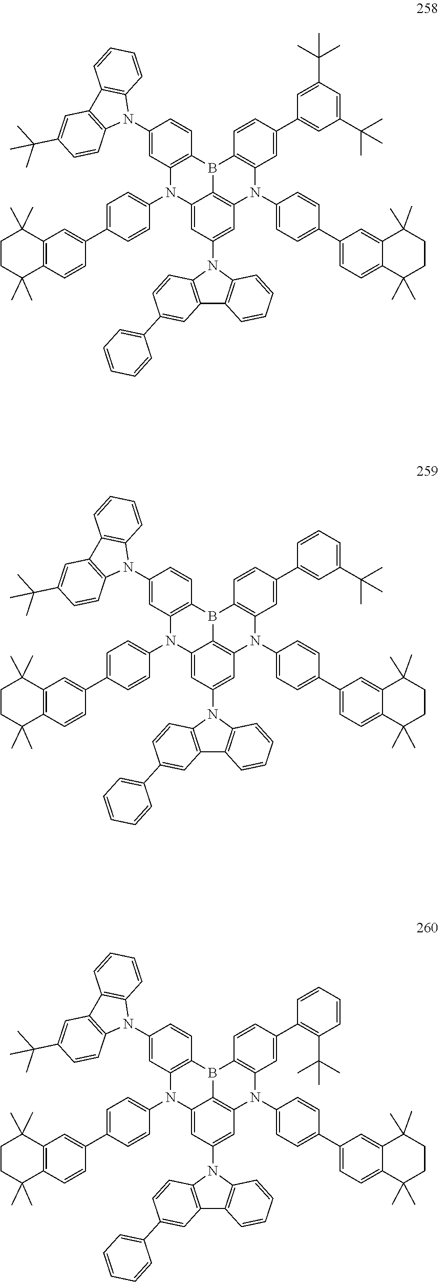

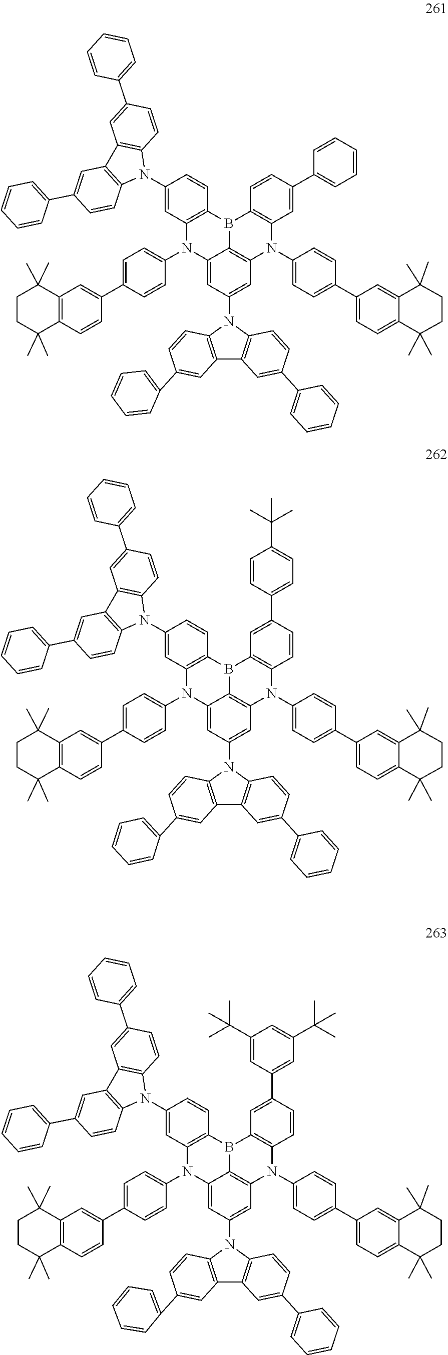

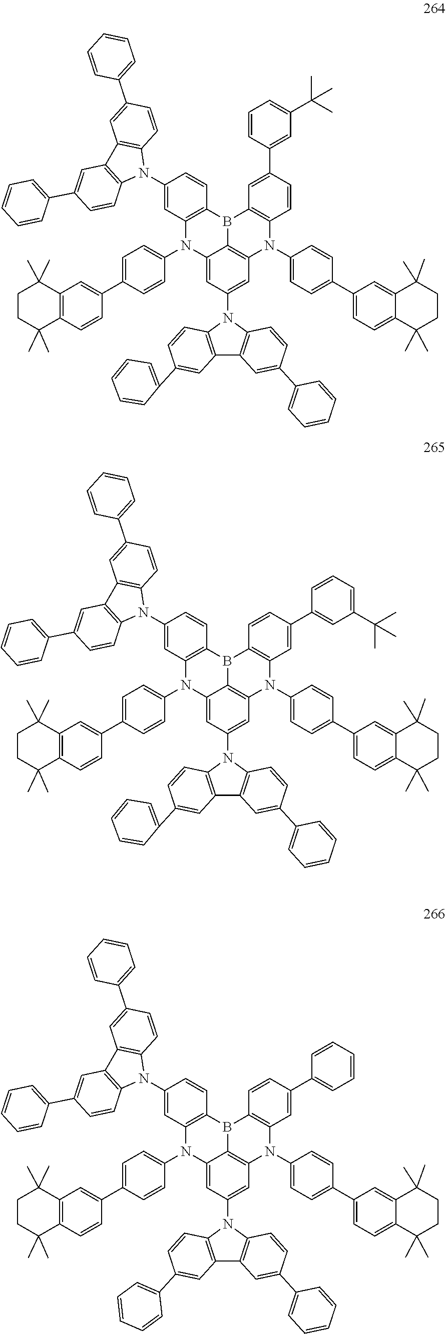

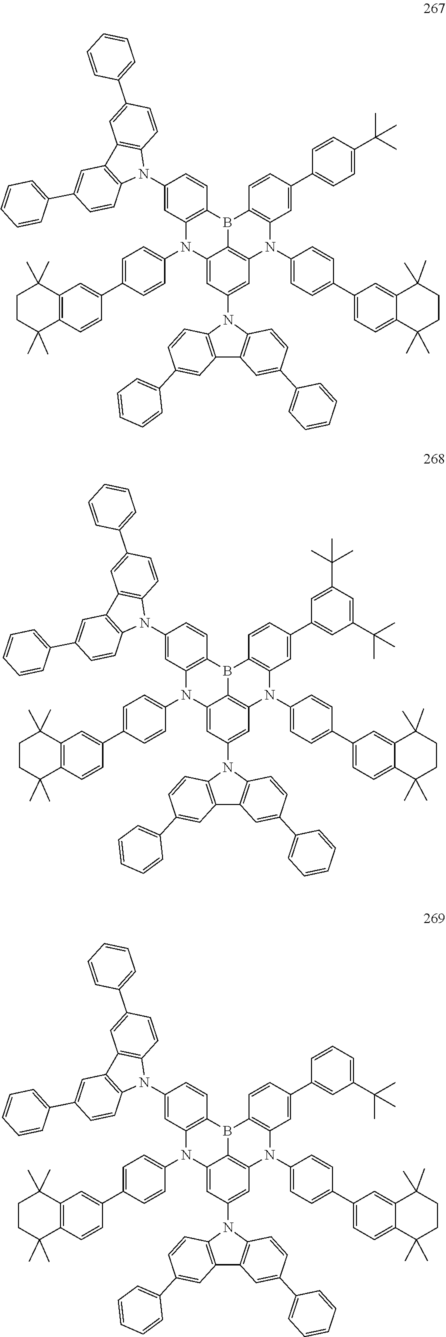

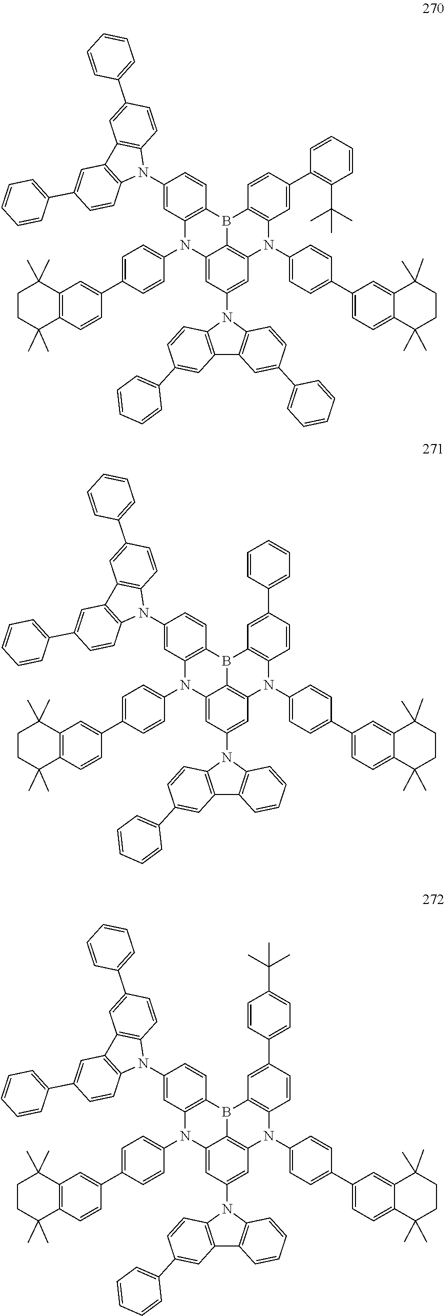

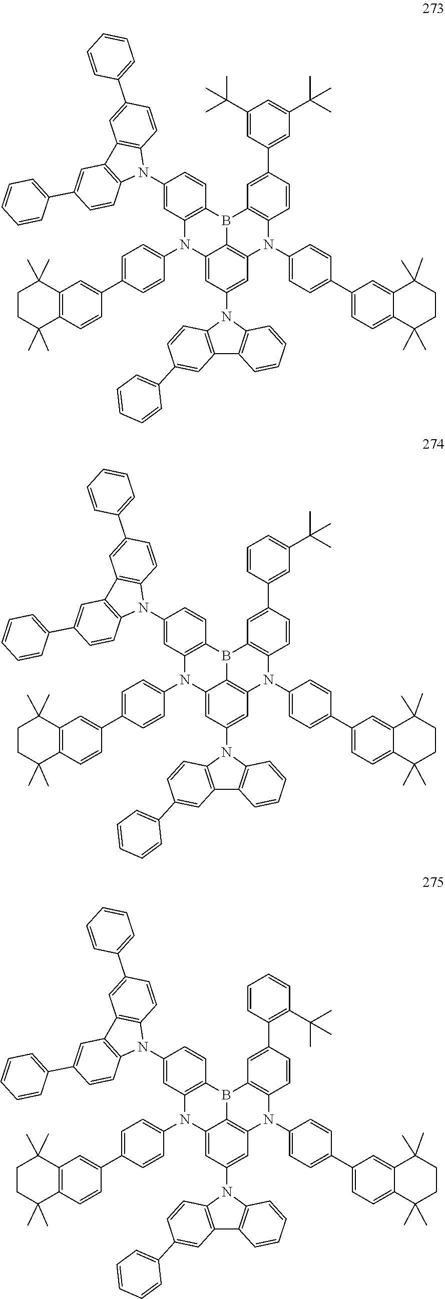

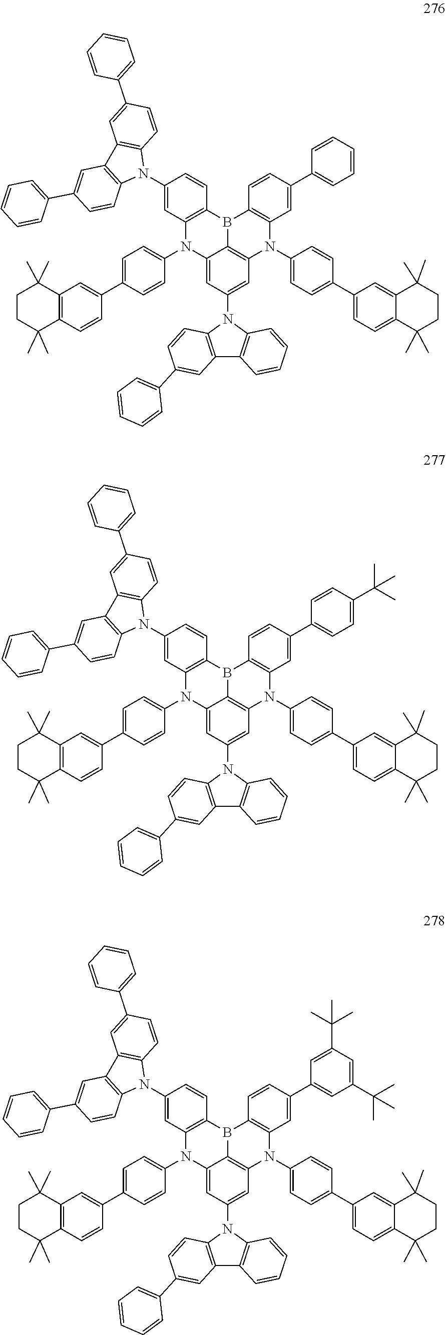

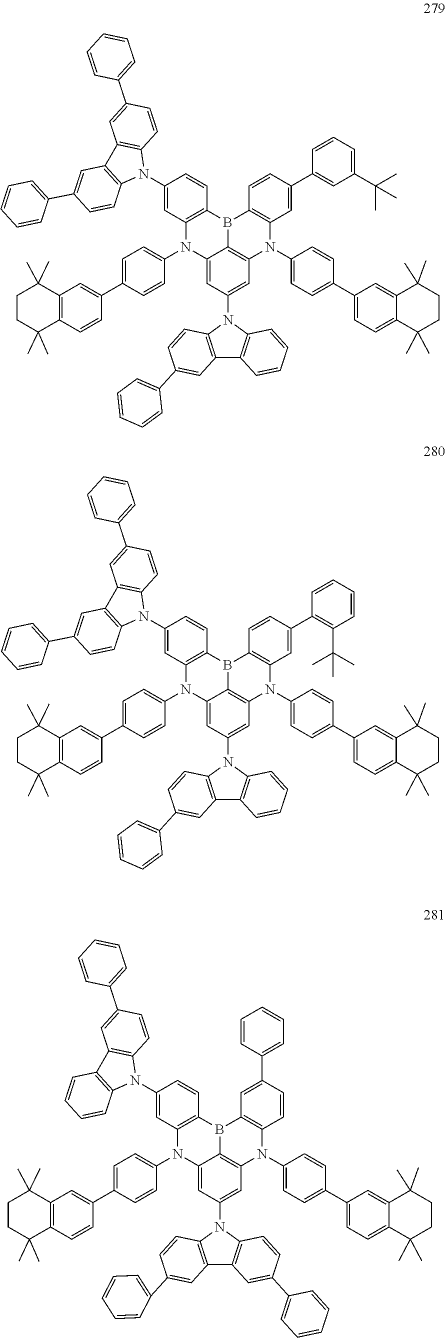

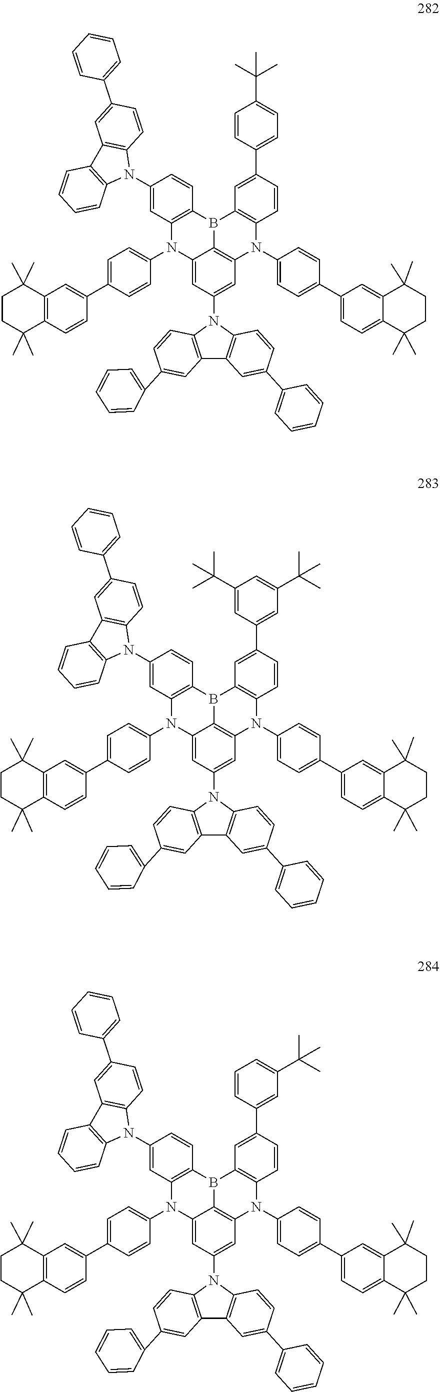

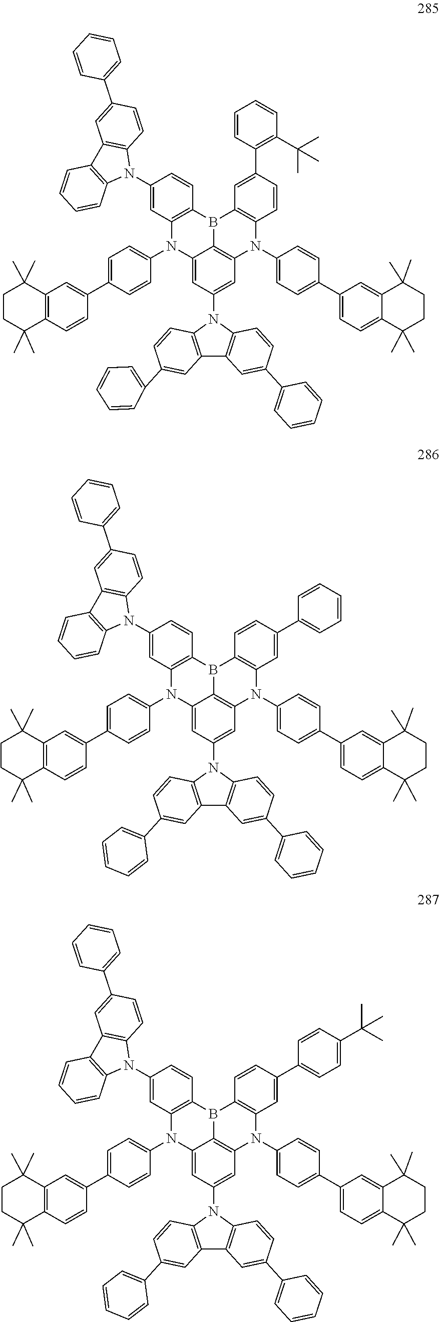

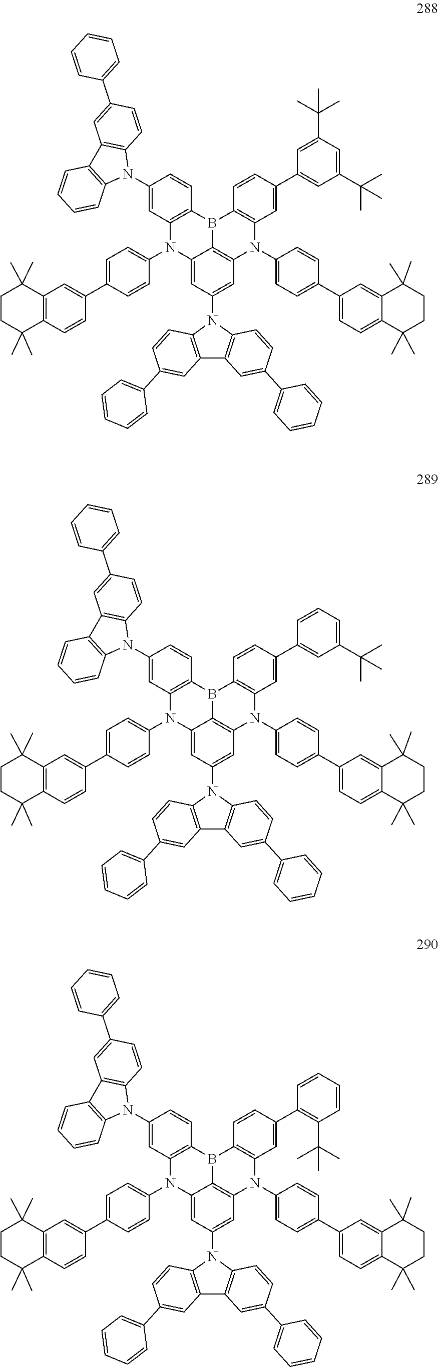

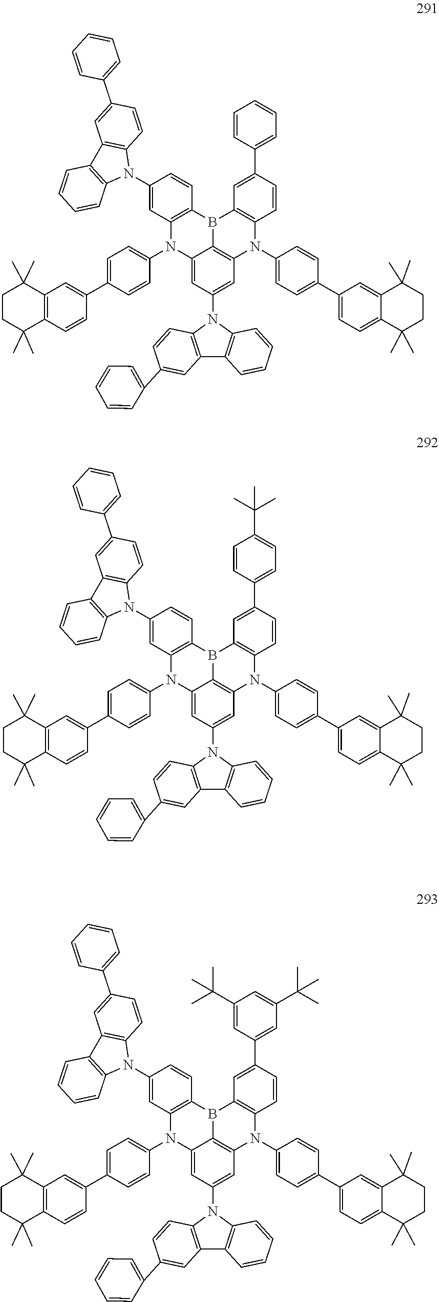

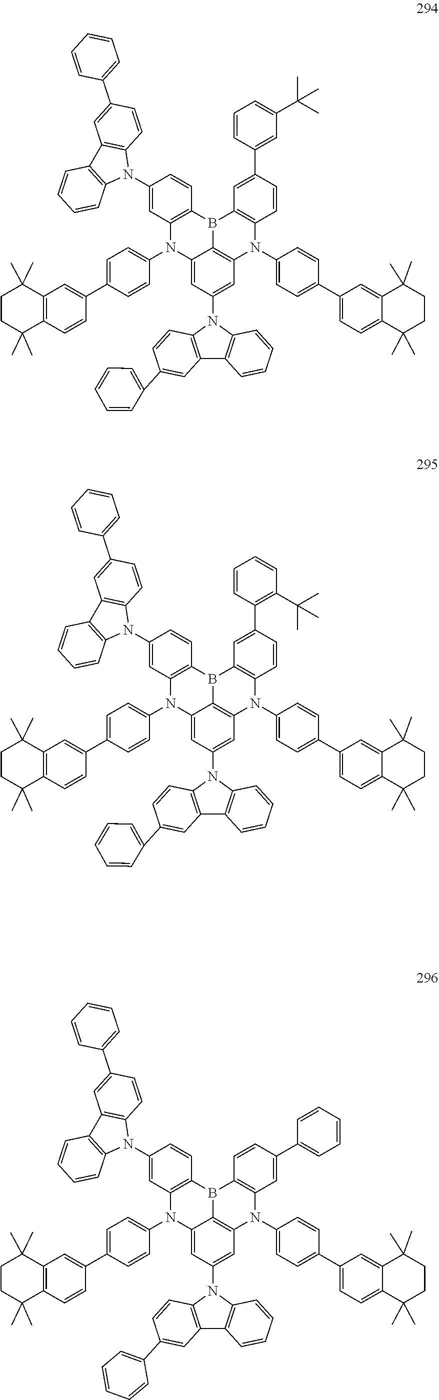

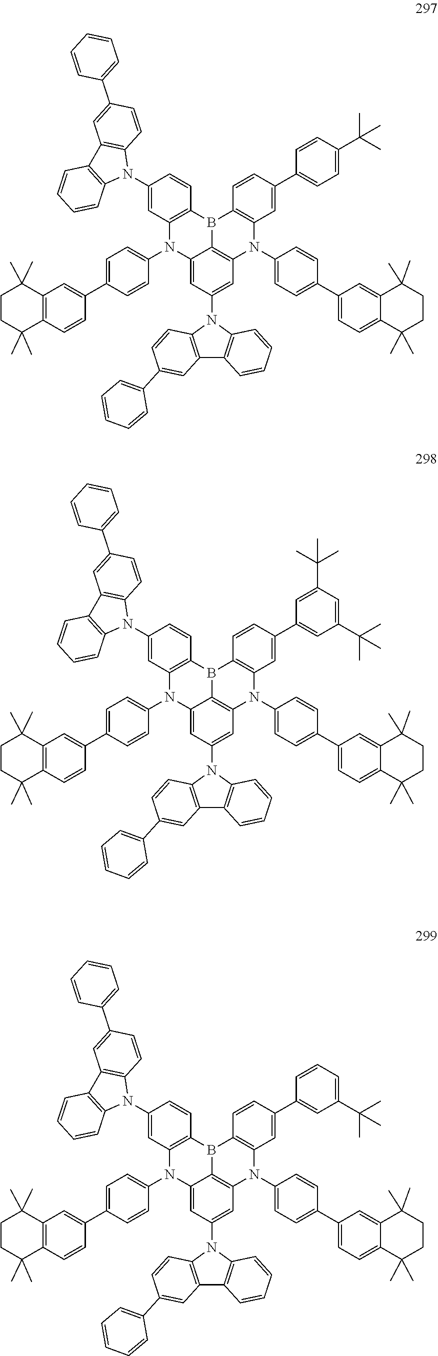

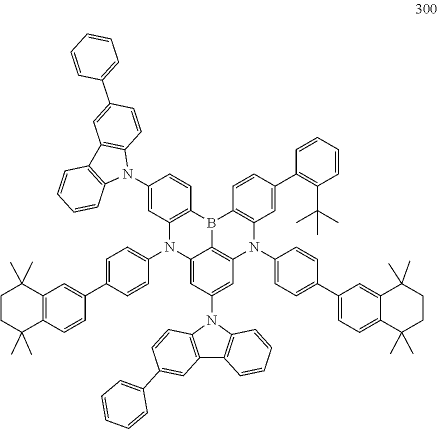 [SMILES: CC(C)(C)c1ccccc1-c1ccc2c(c1)N(c1ccc(-c3ccc4c(c3)C(C)(C)CCC4(C)C)cc1)c1cc(-n3c4ccccc4c4cc(-c5ccccc5)ccc43)cc3c1B2c1ccc(-n2c4ccccc4c4cc(-c5ccccc5)ccc42)cc1N3c1ccc(-c2ccc3c(c2)C(C)(C)CCC3(C)C)cc1]

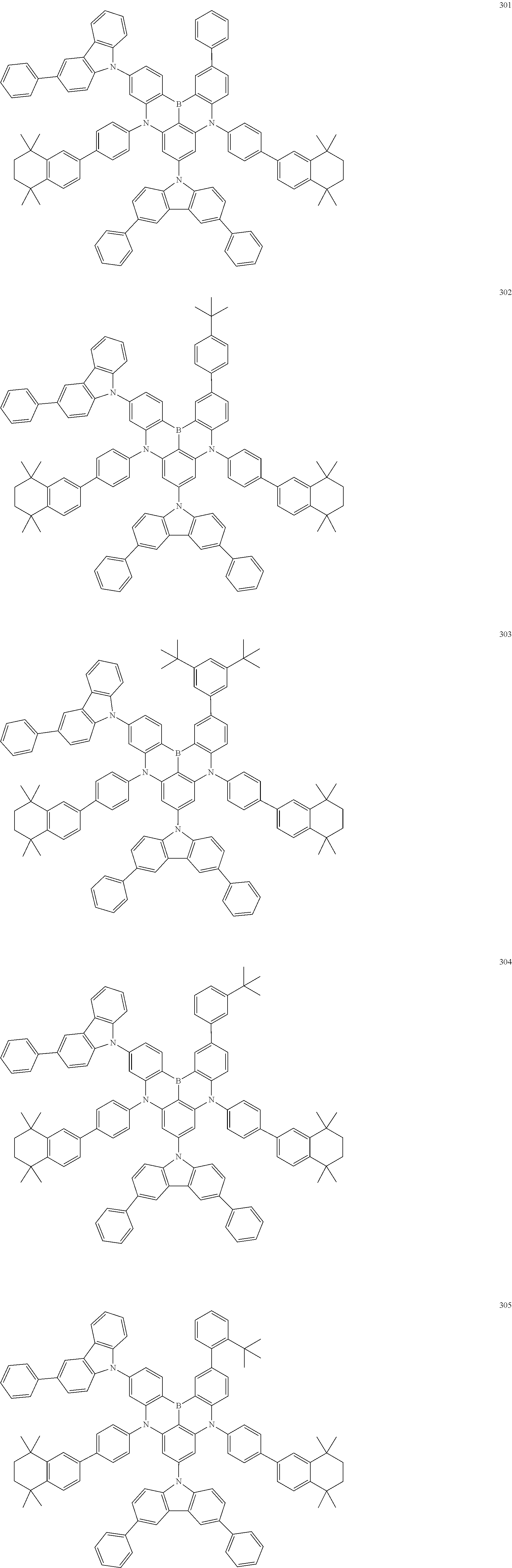

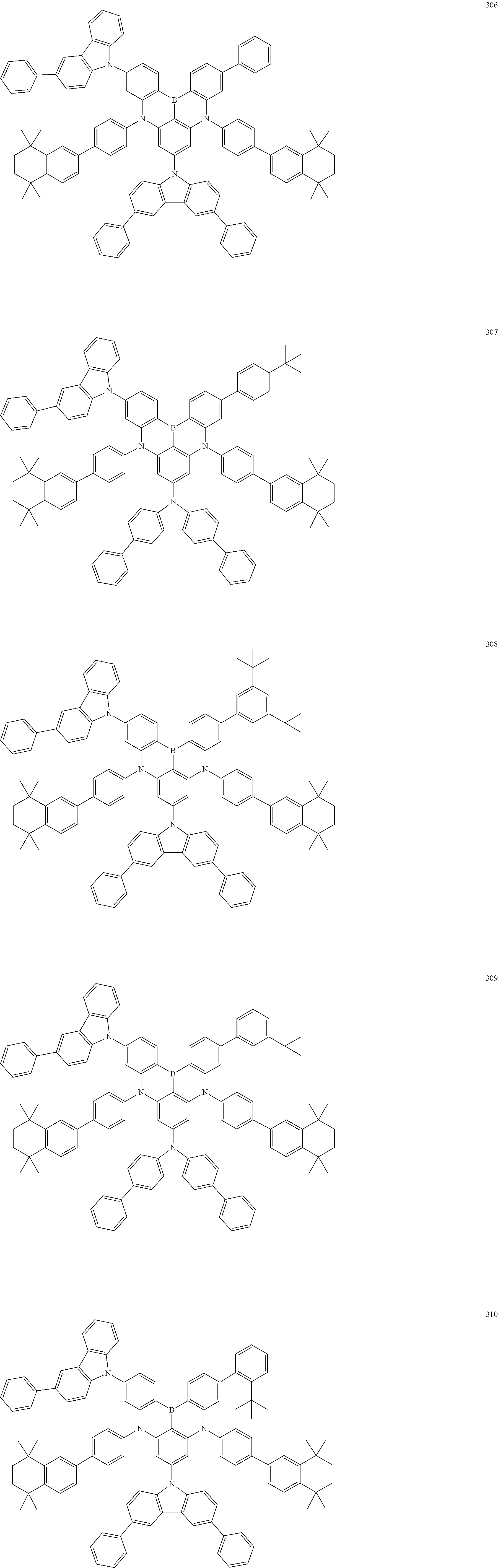

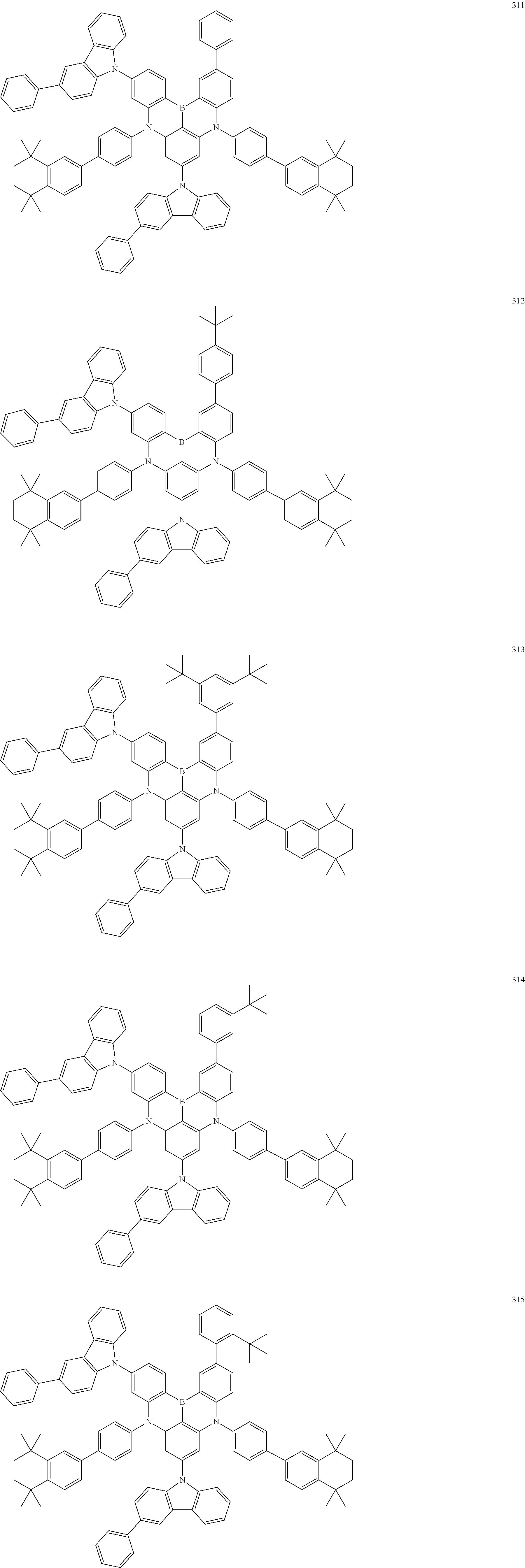

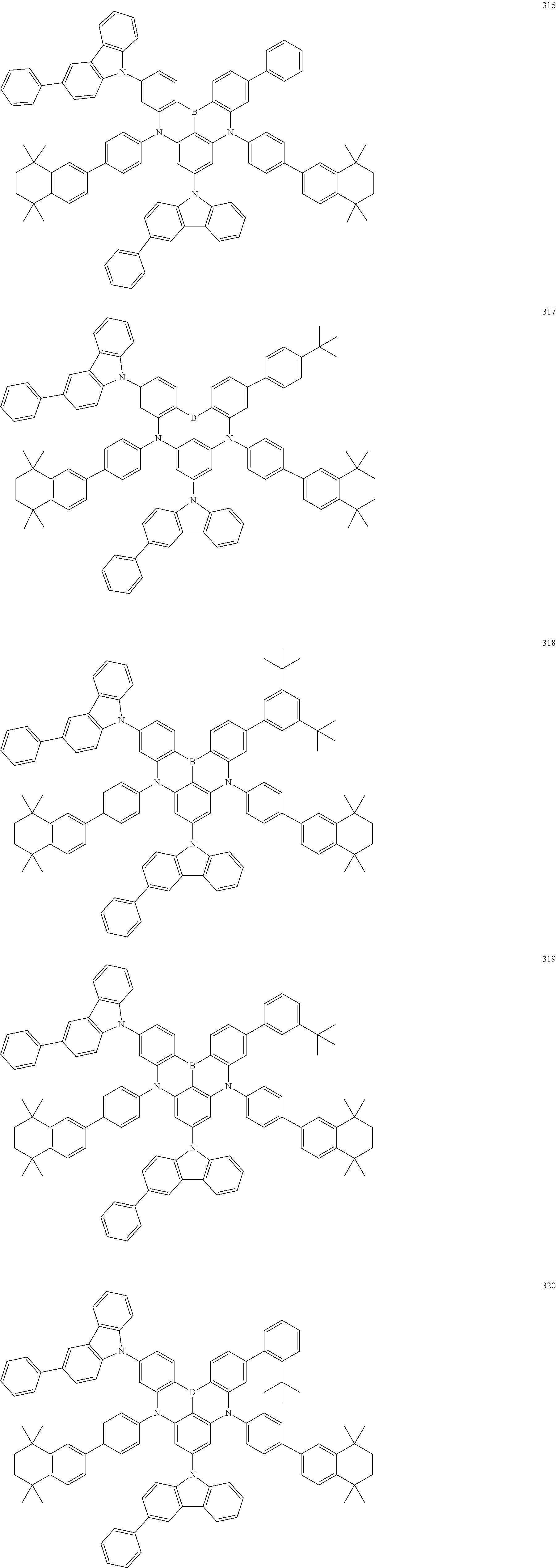

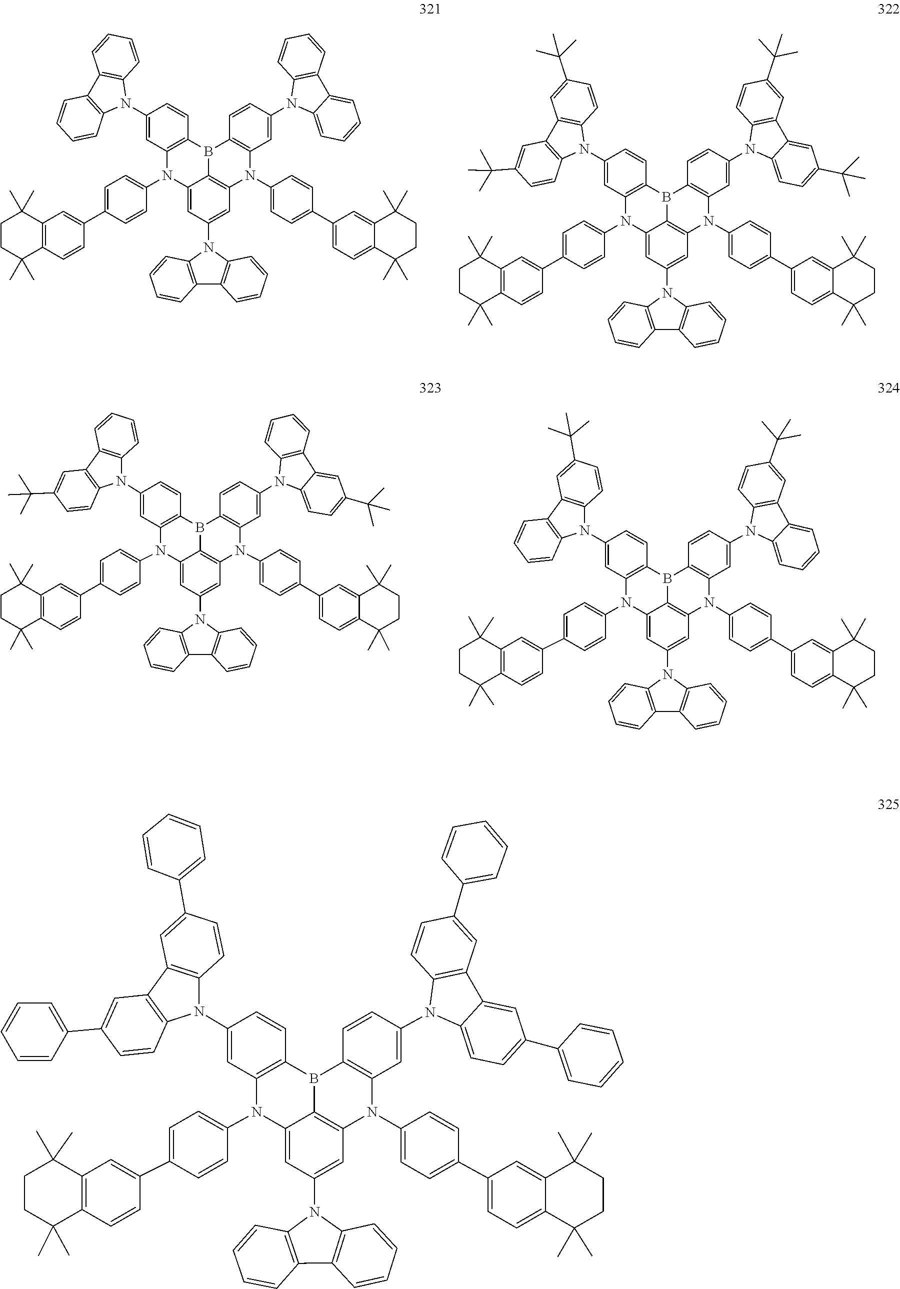

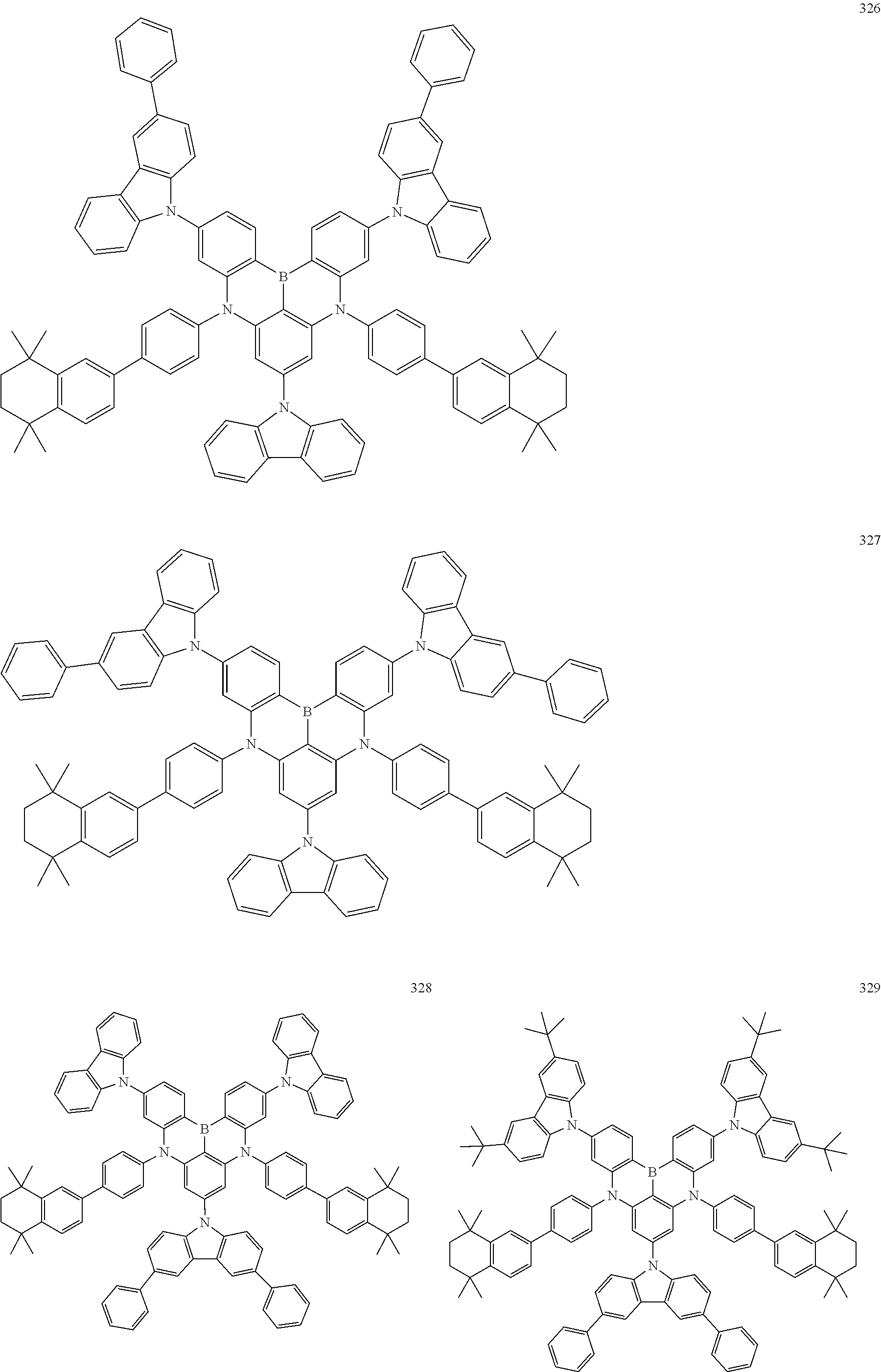

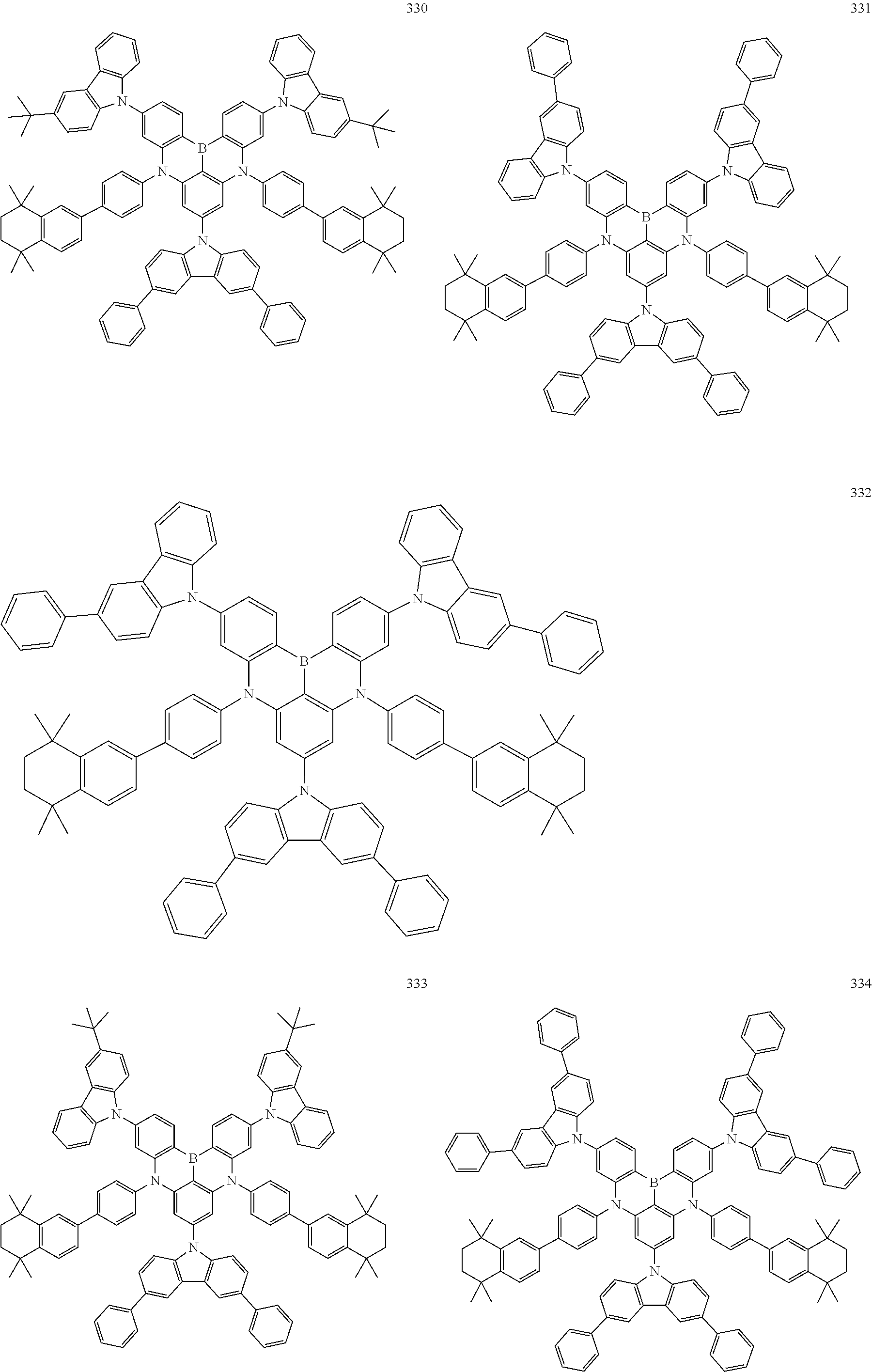

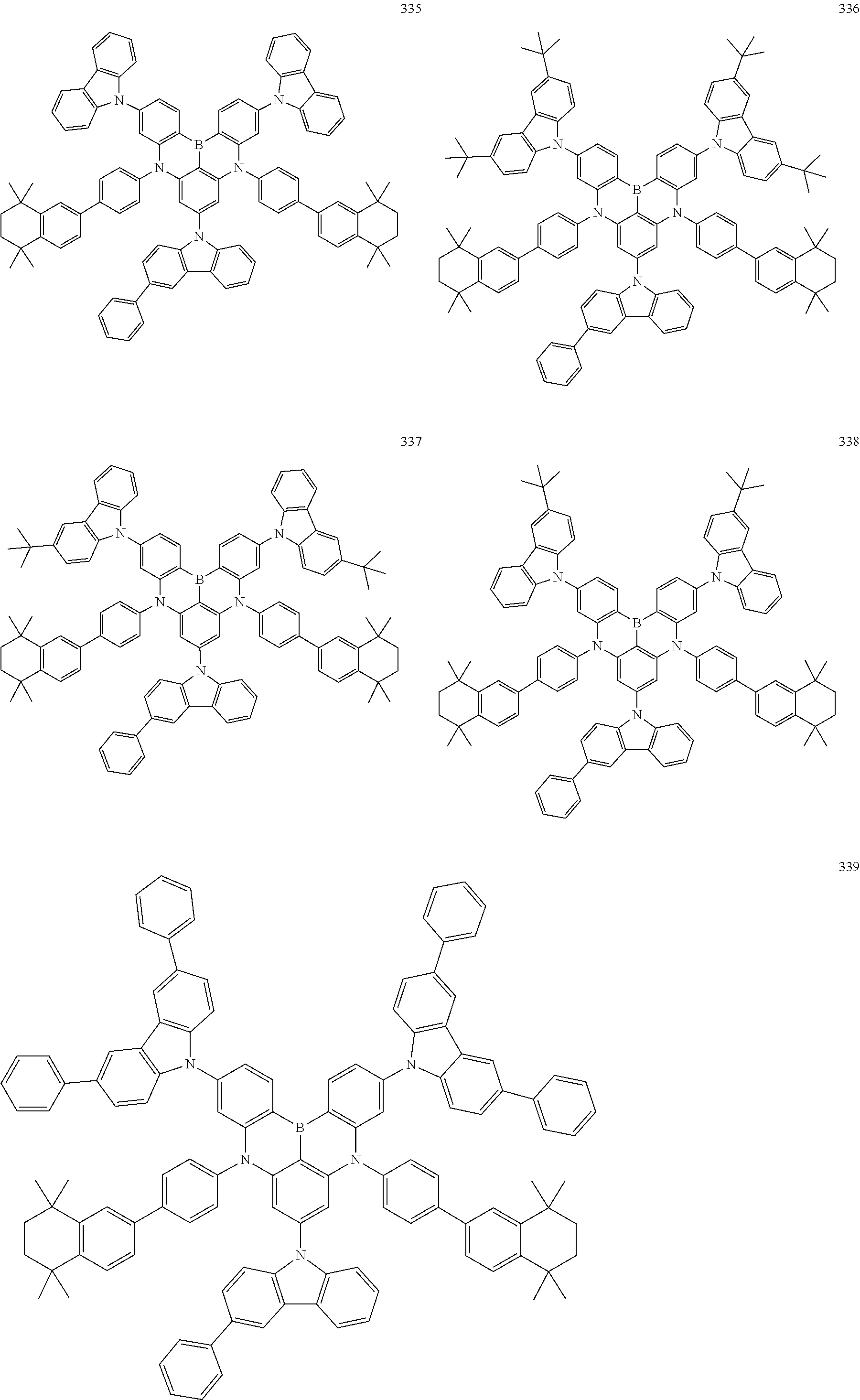

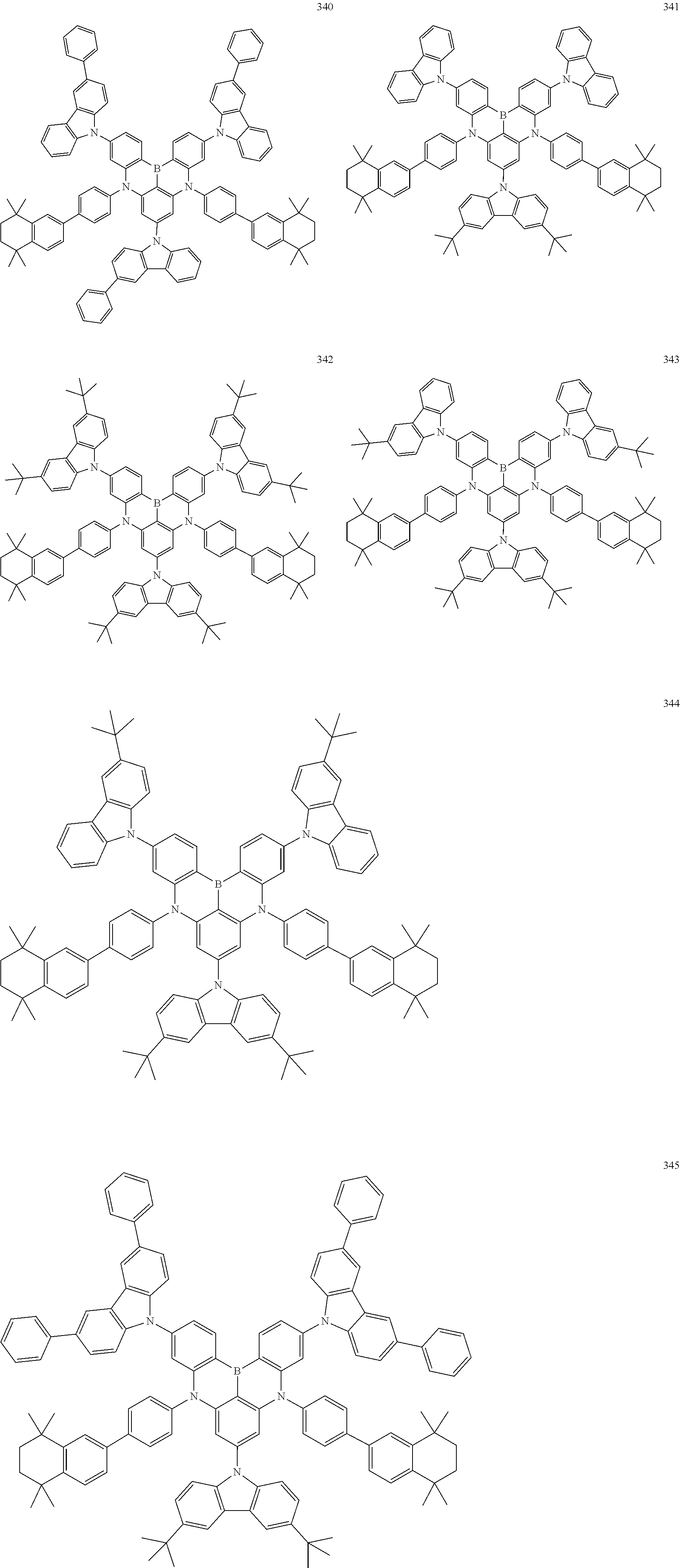

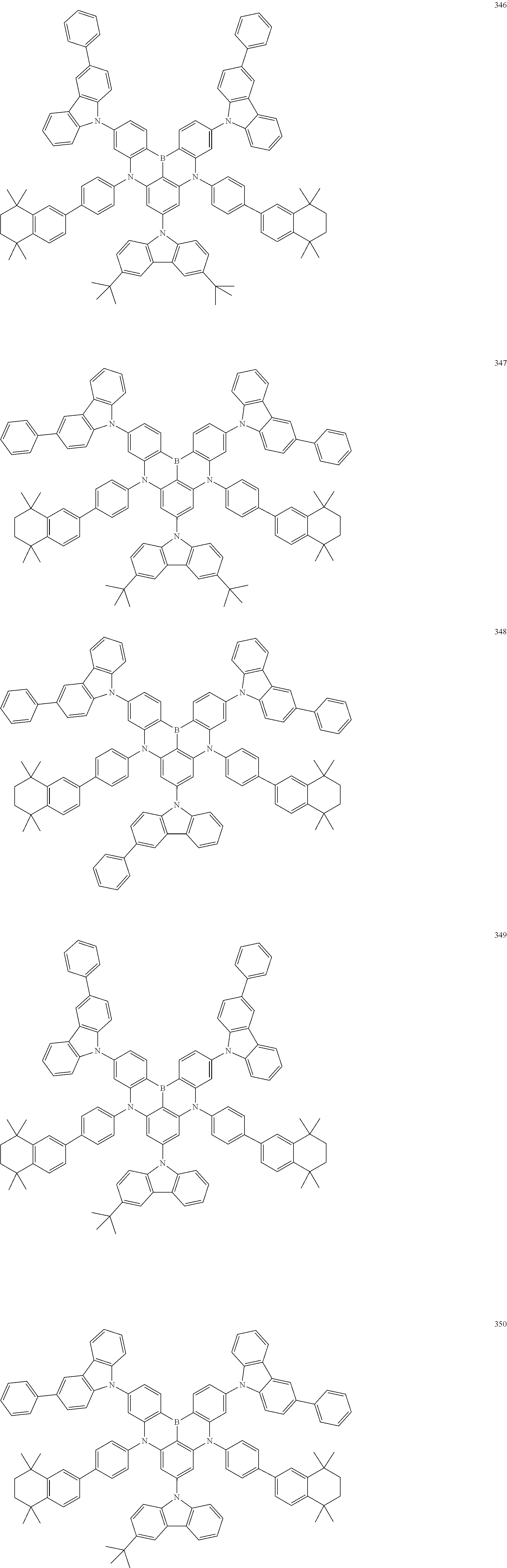

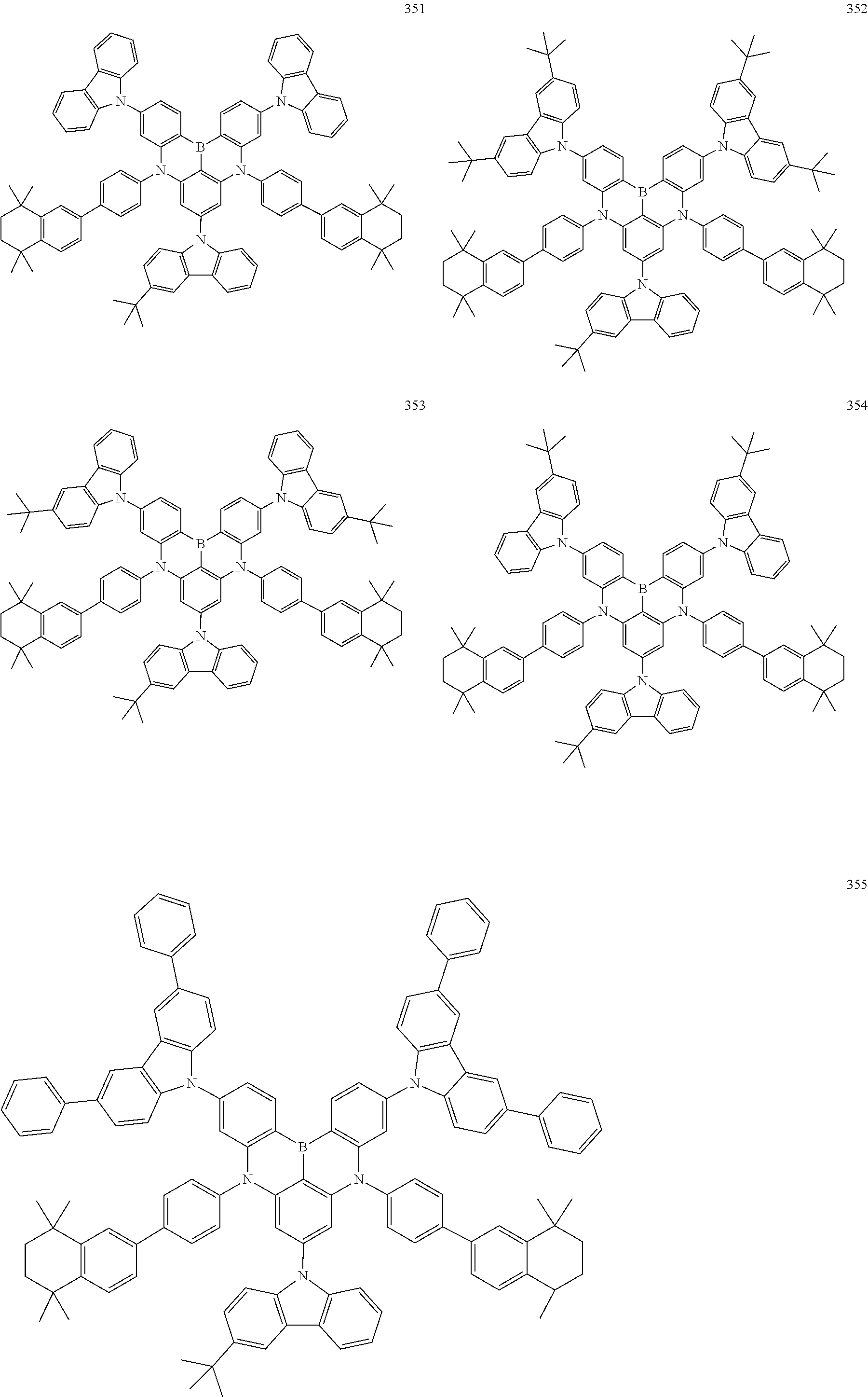

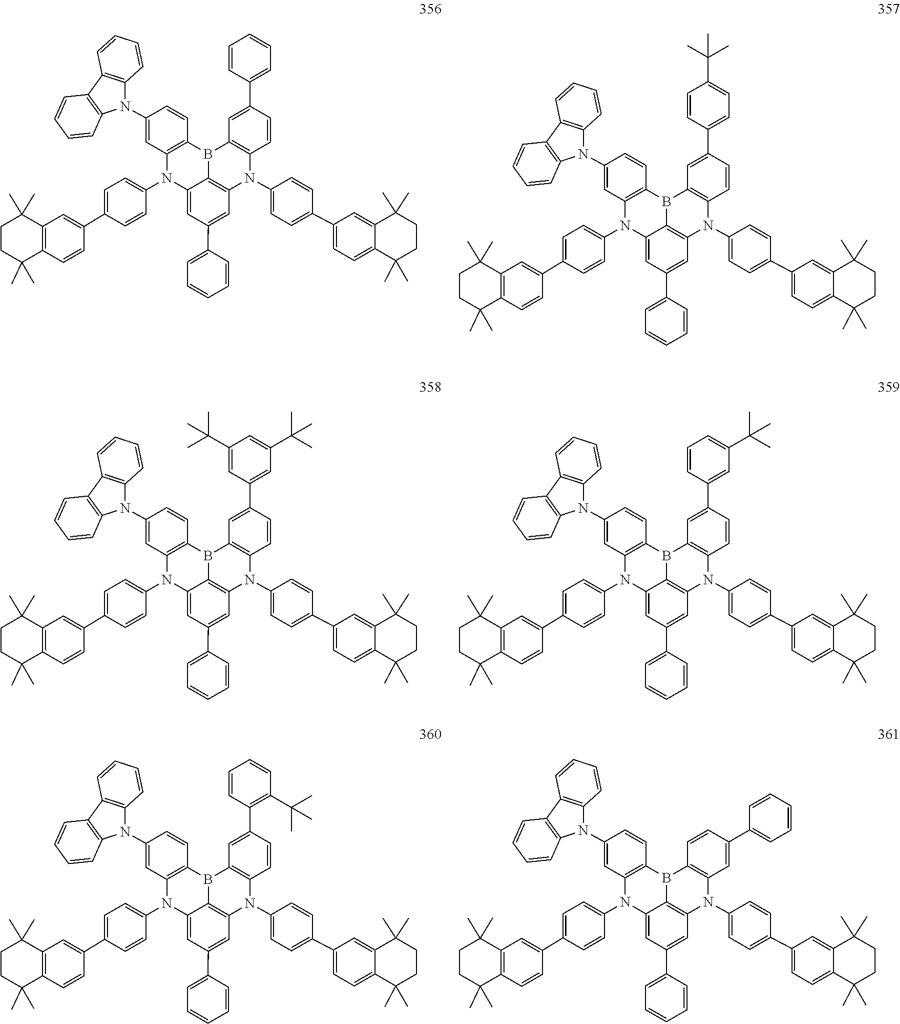

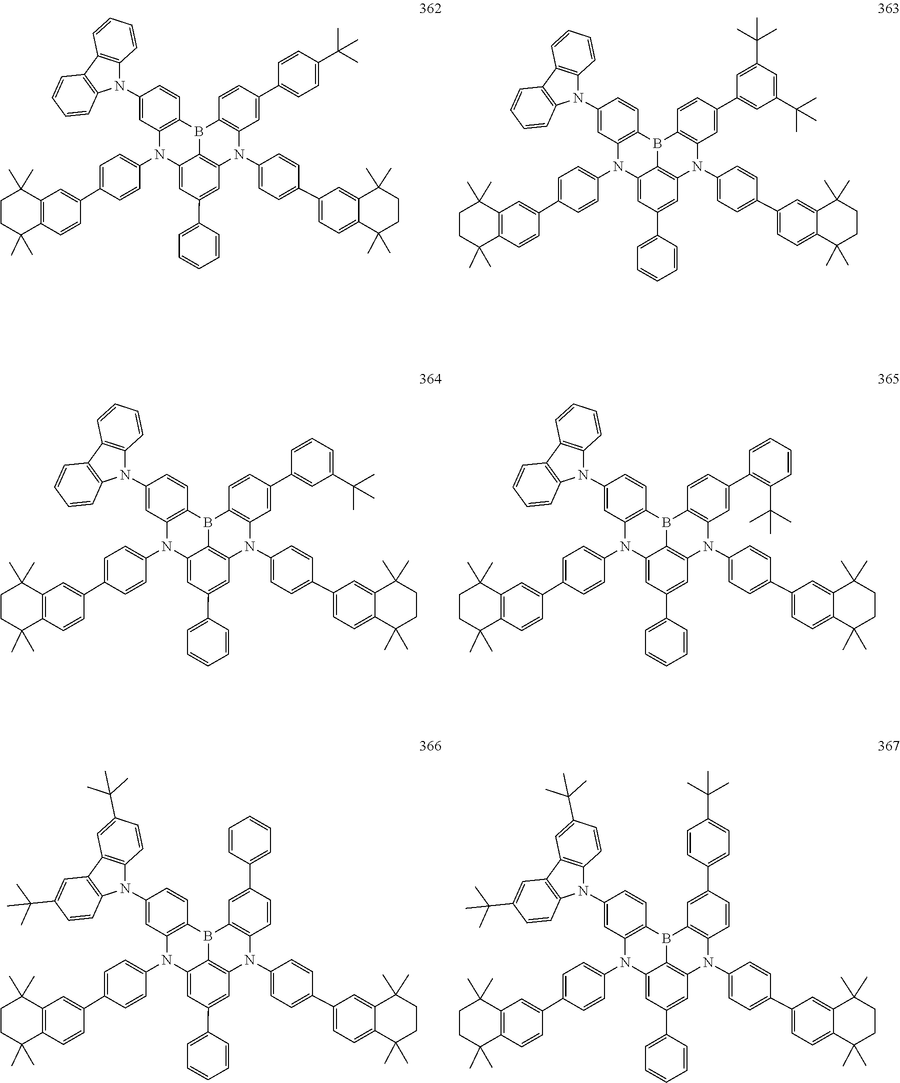

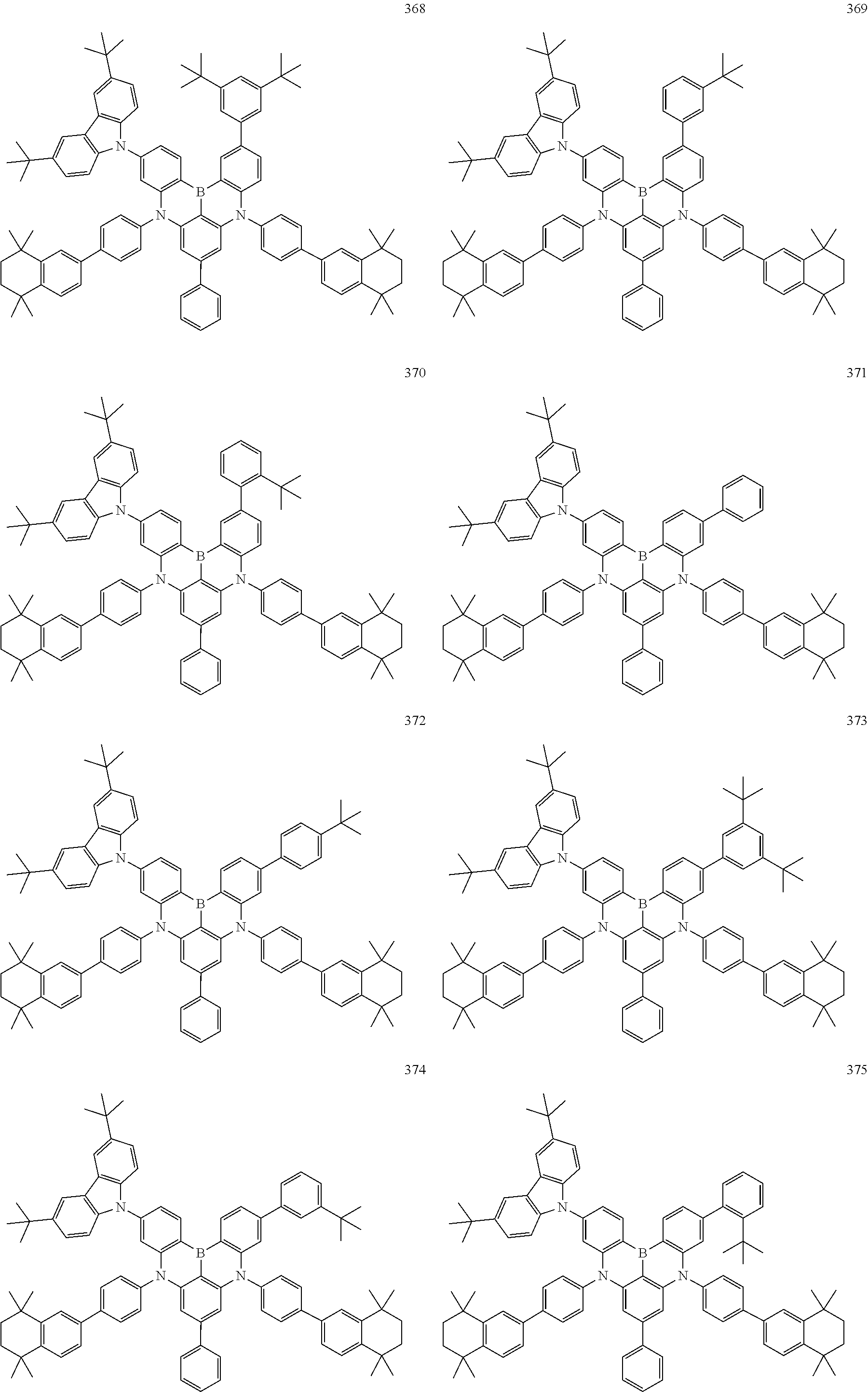

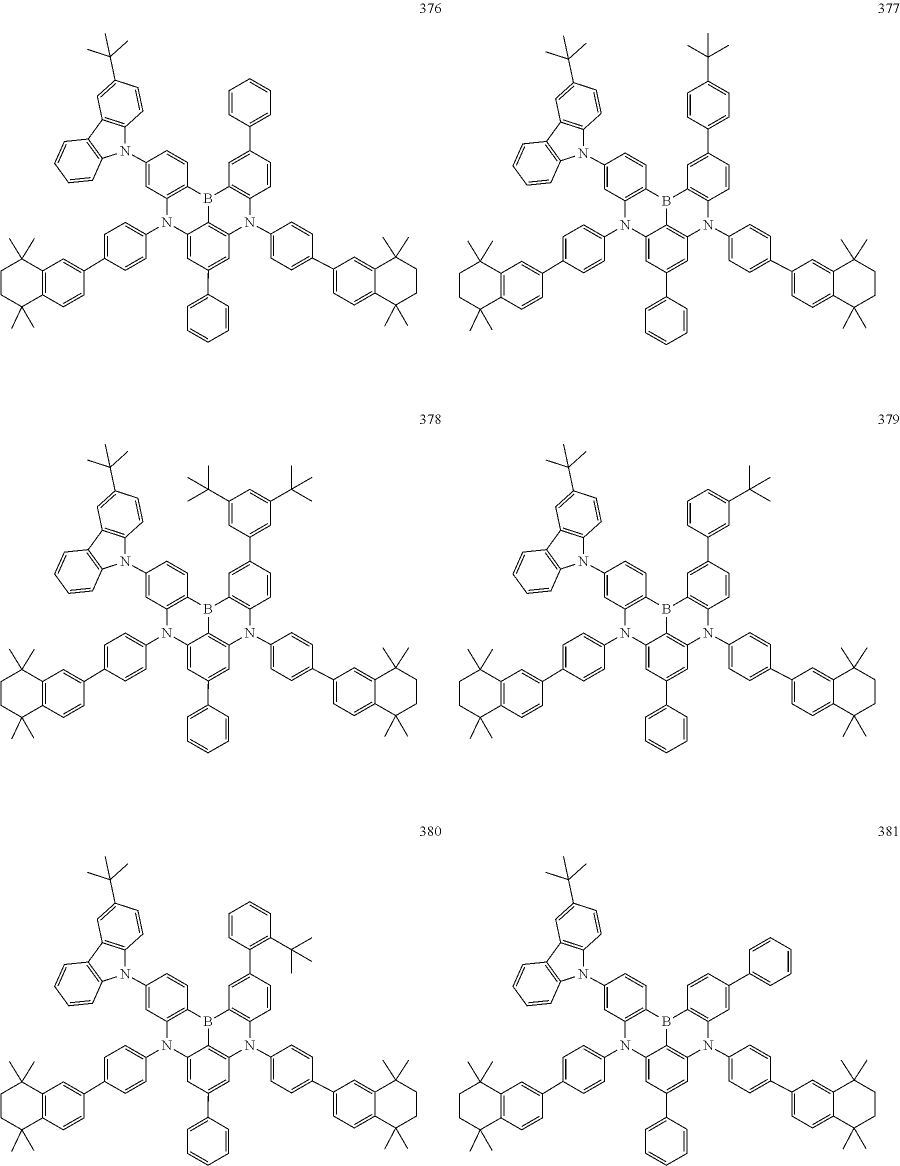

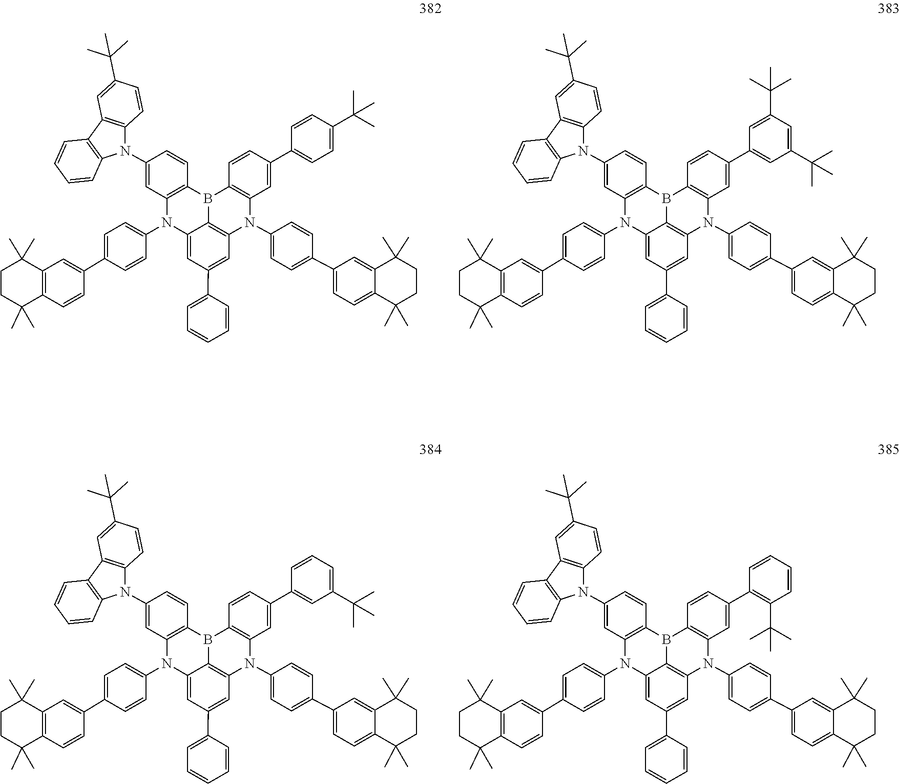

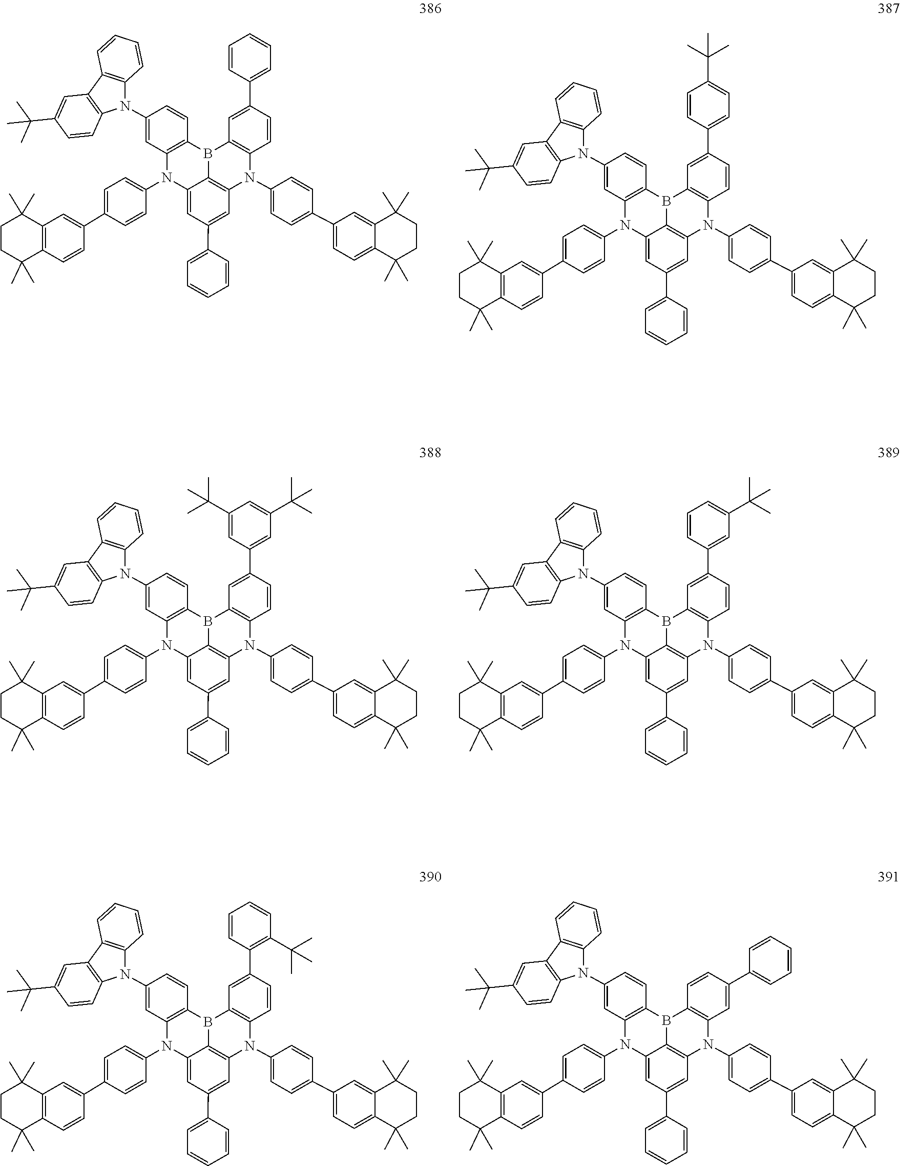

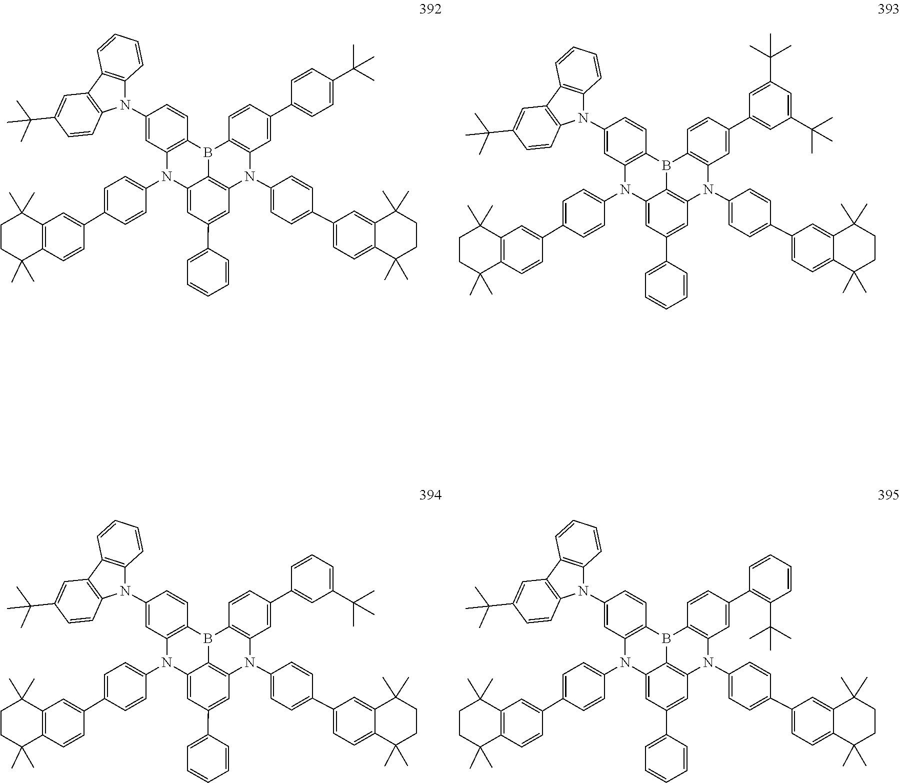

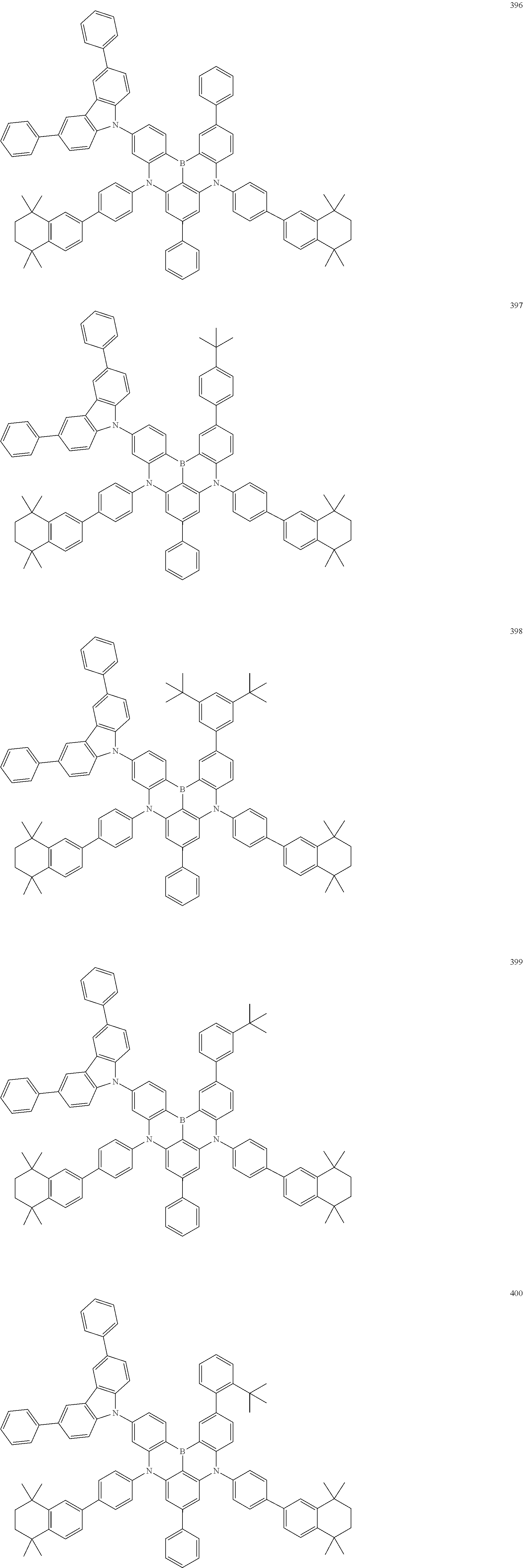

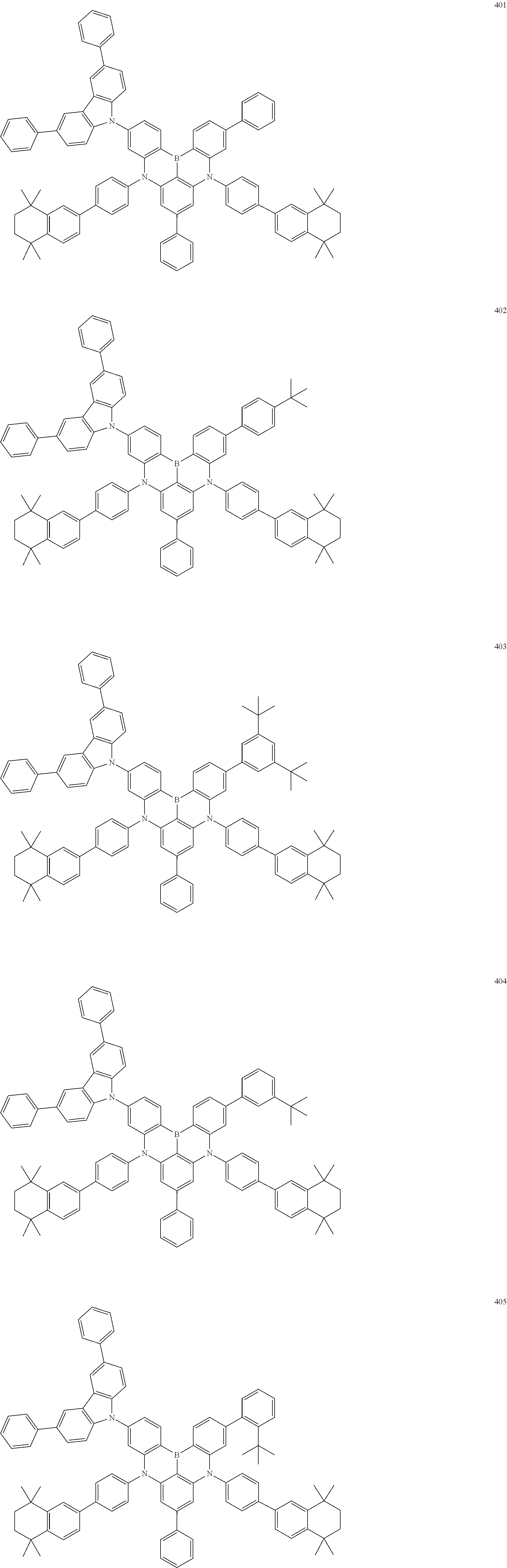

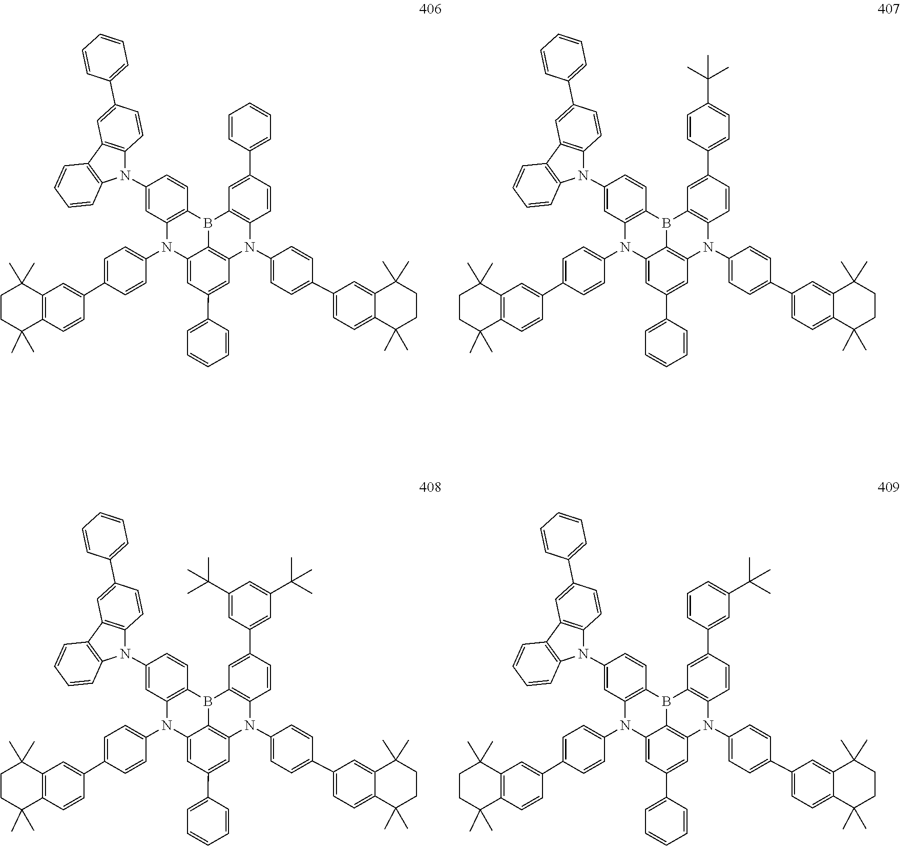

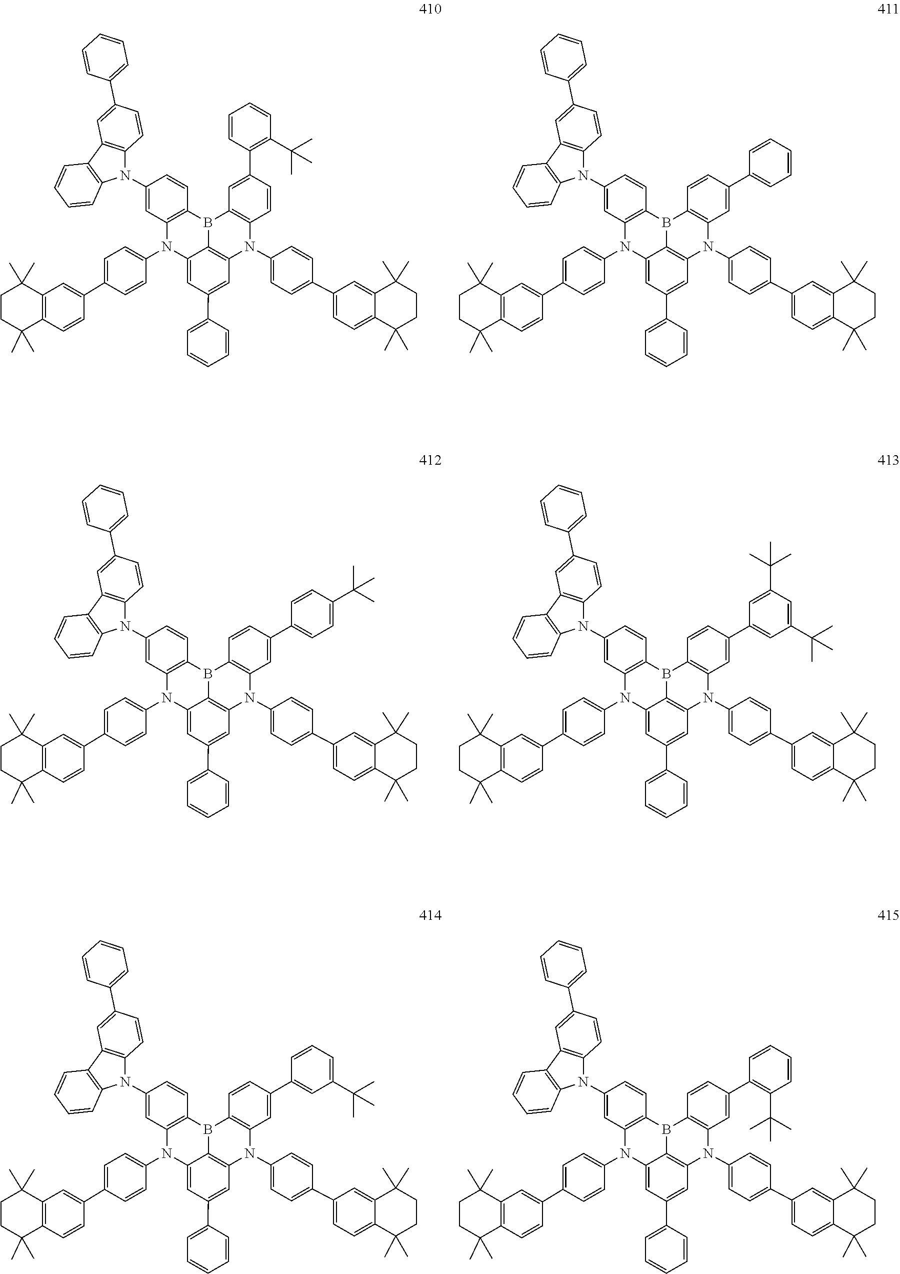

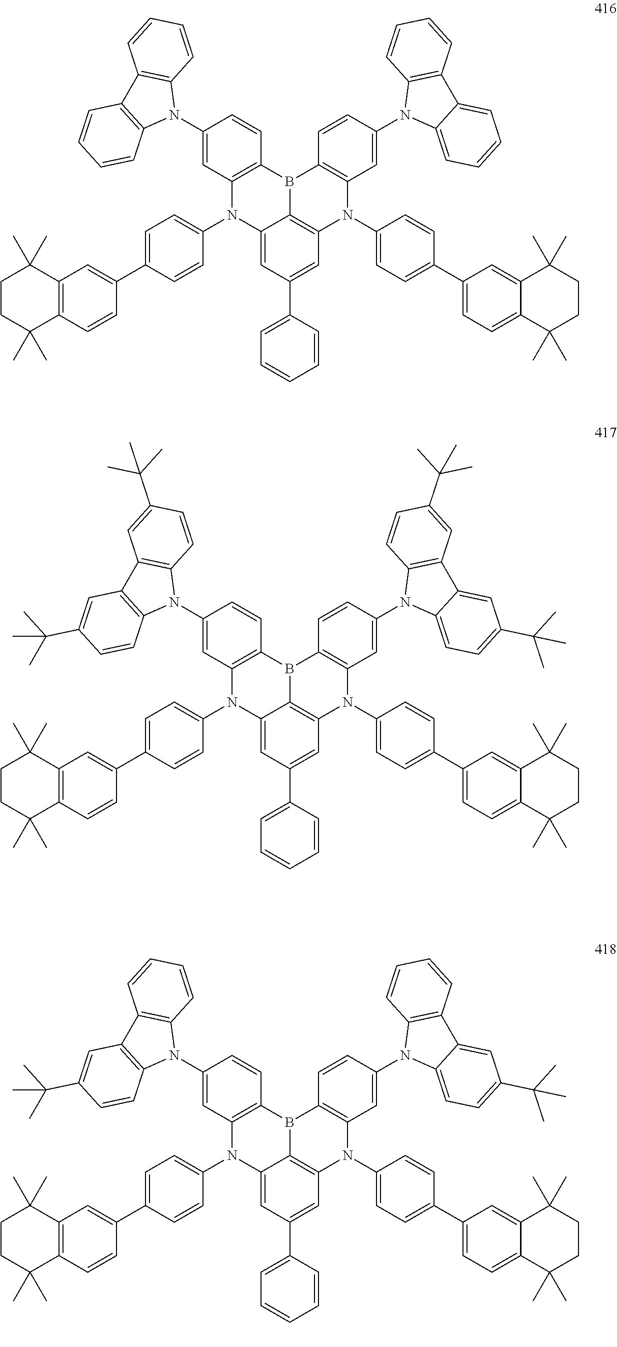

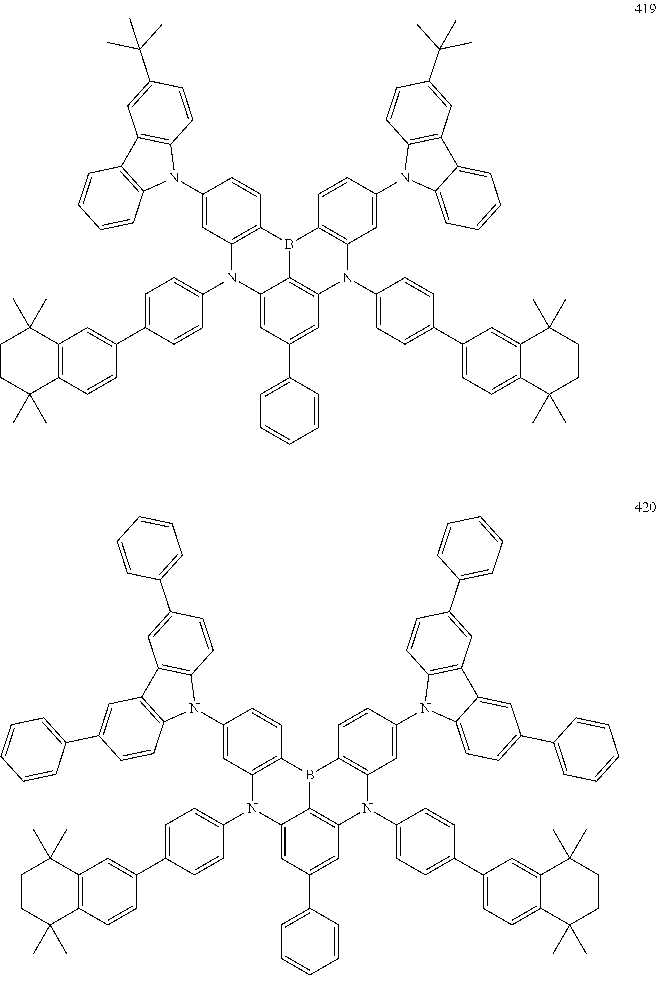

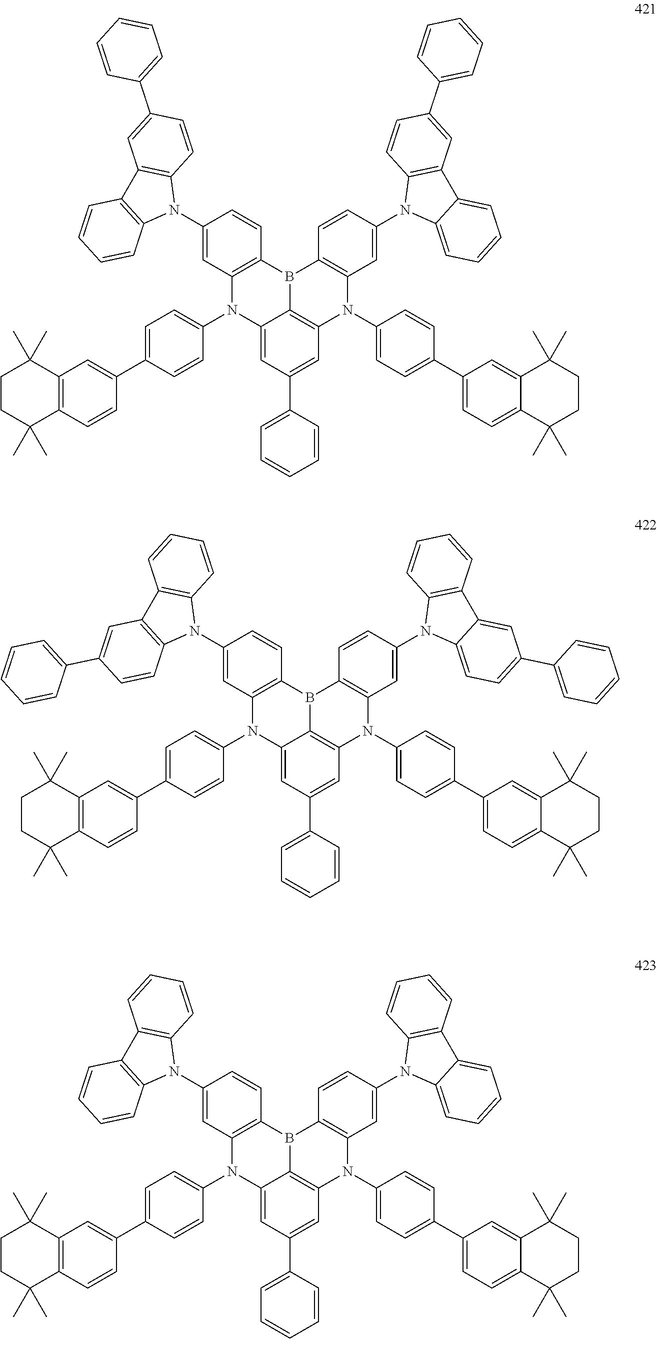

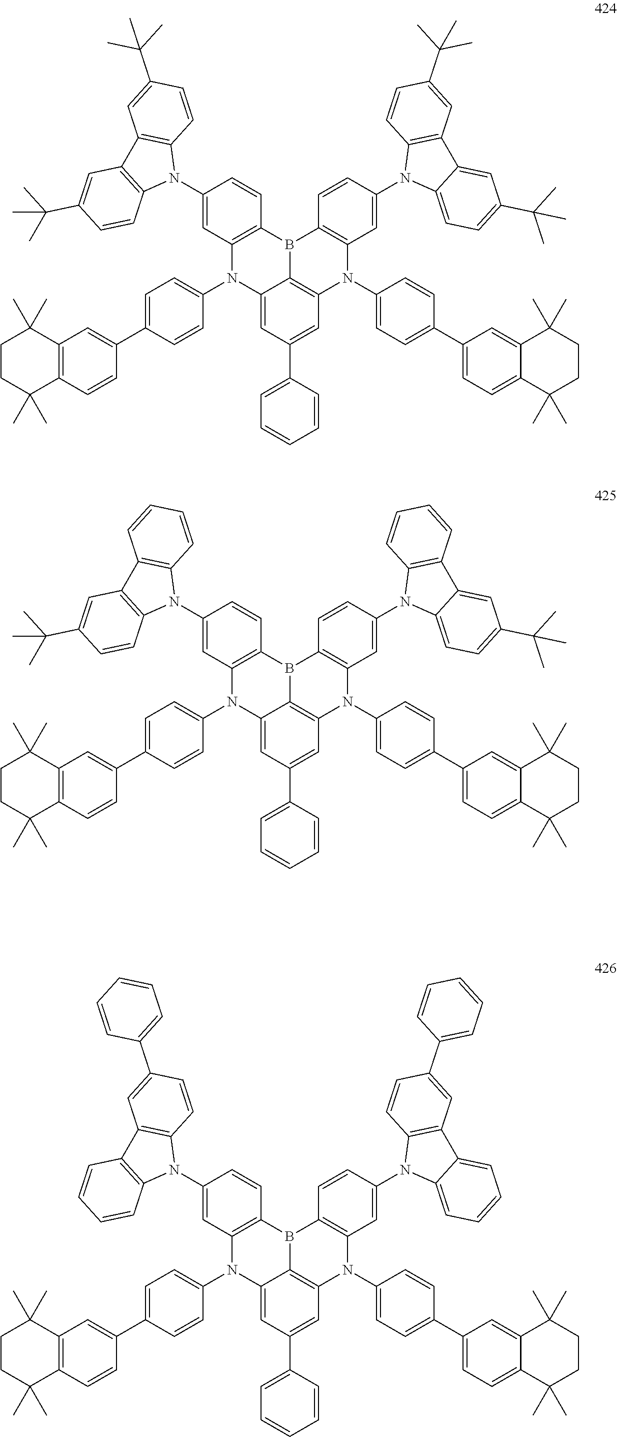

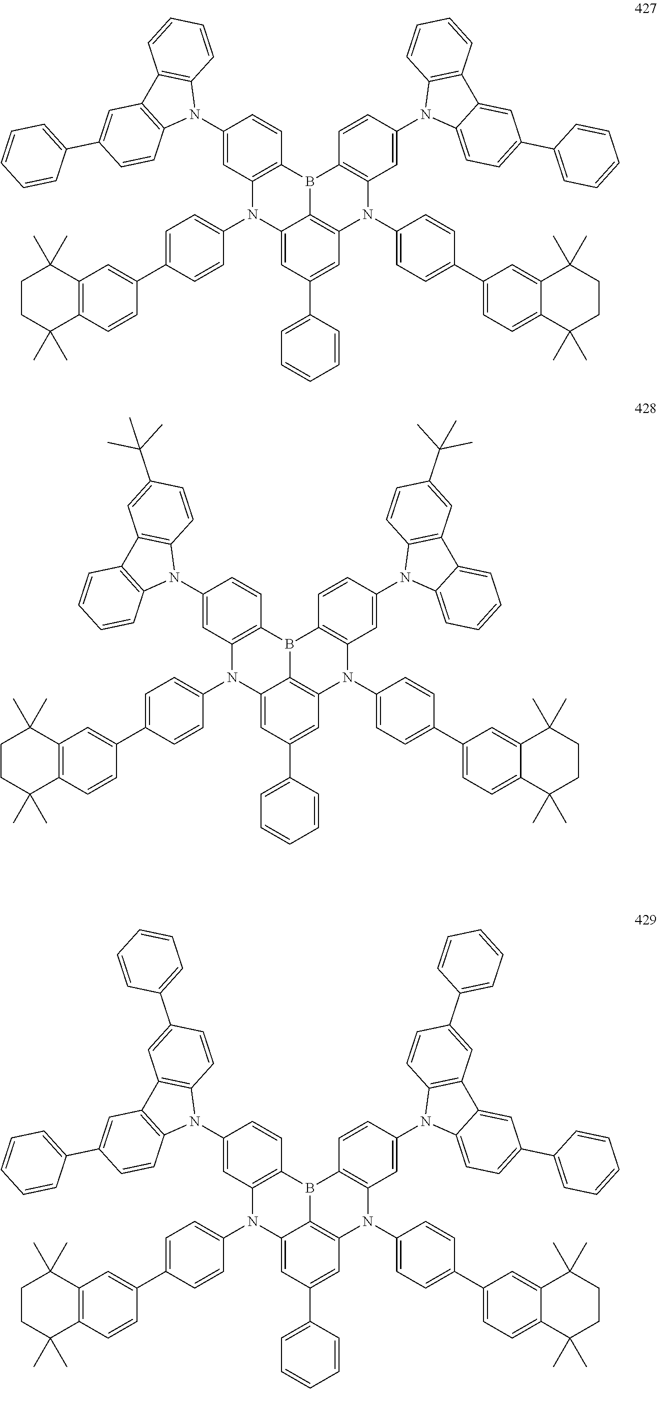

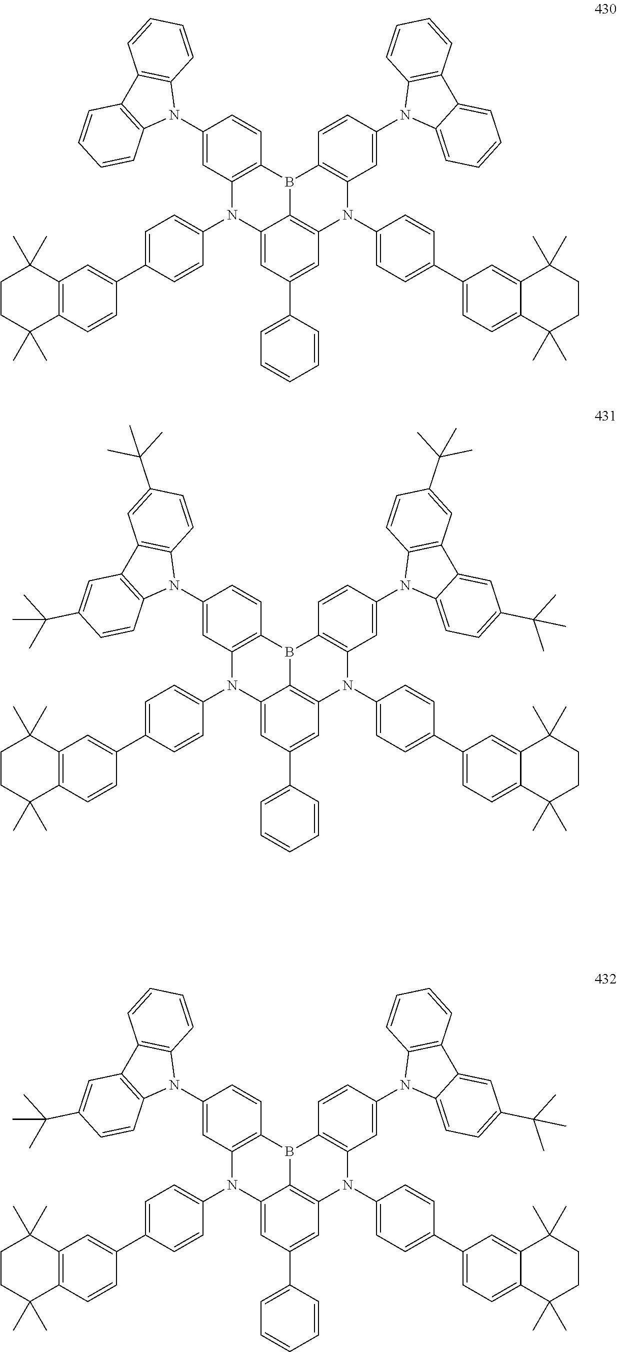

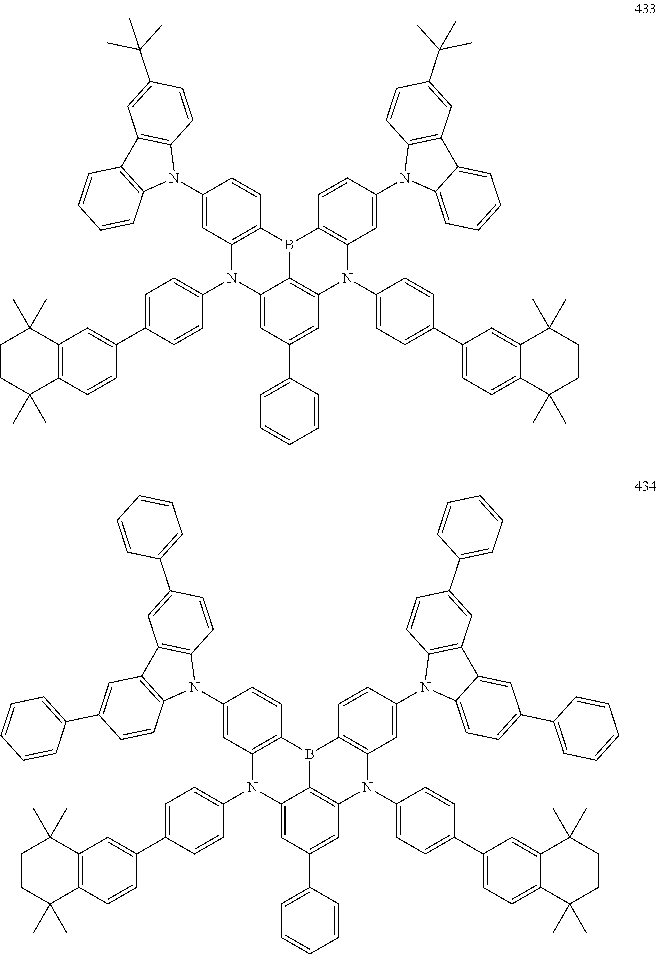

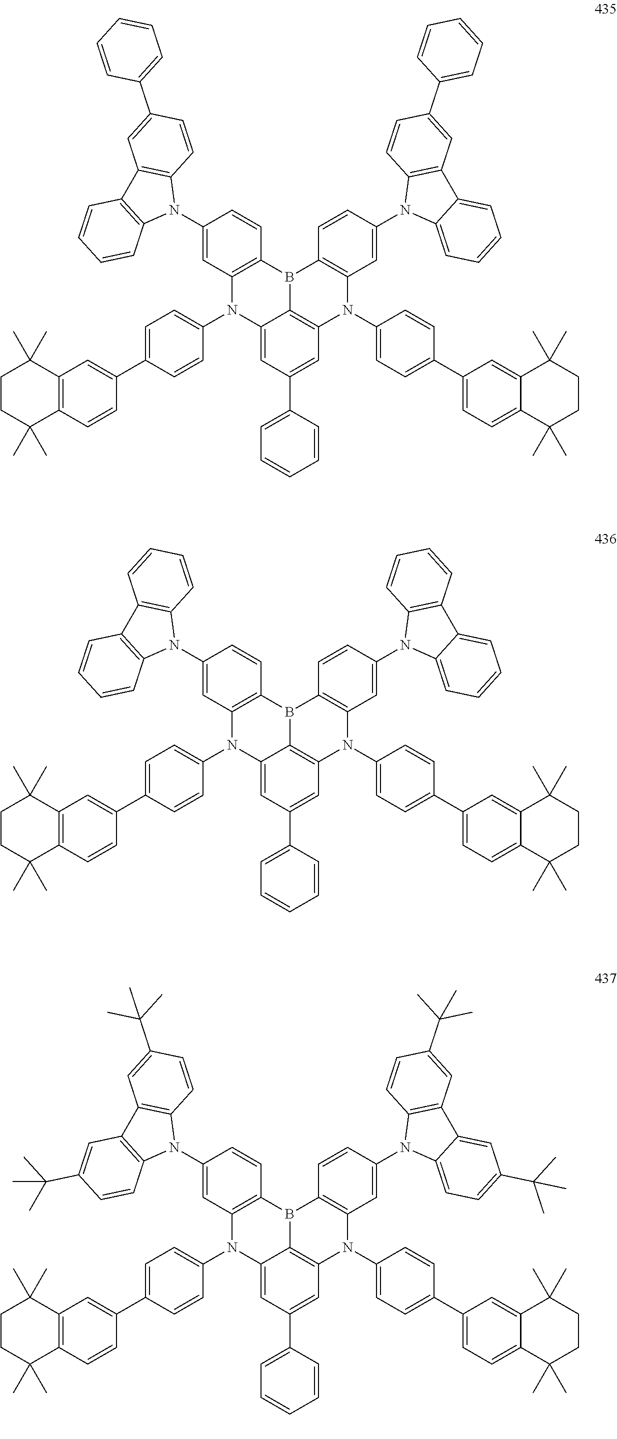

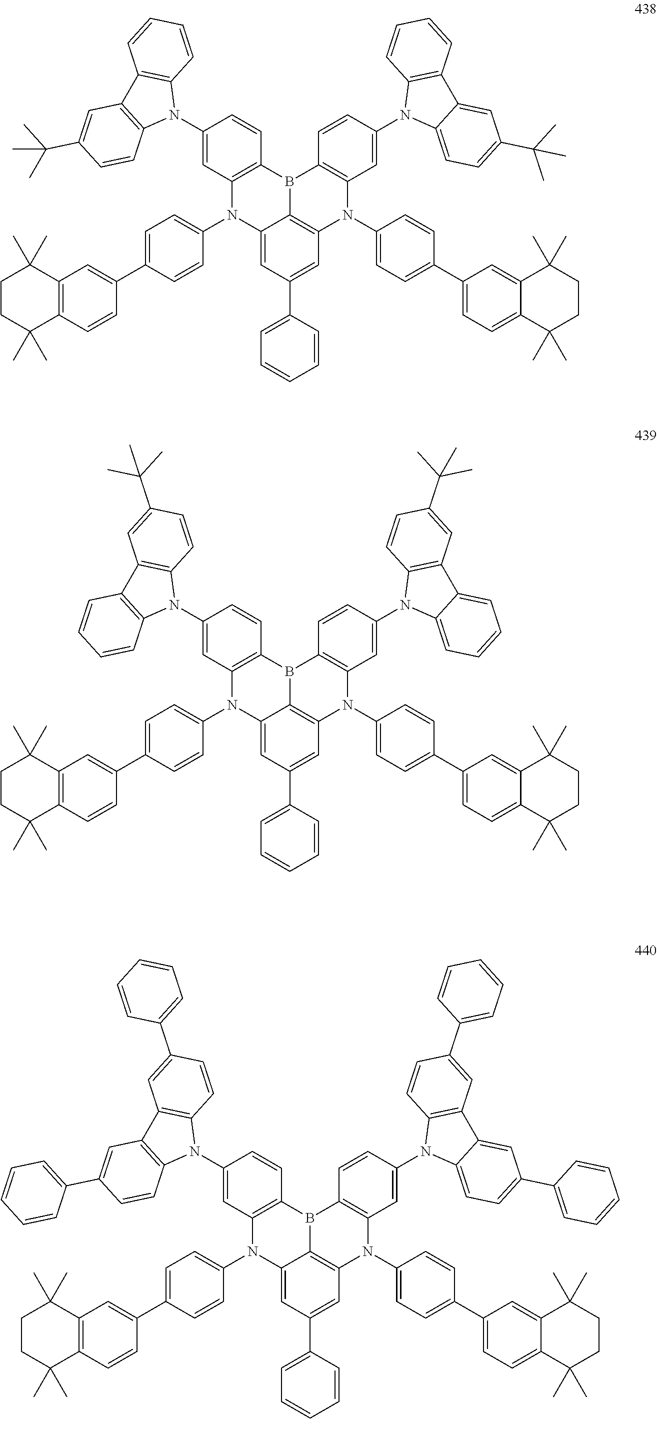

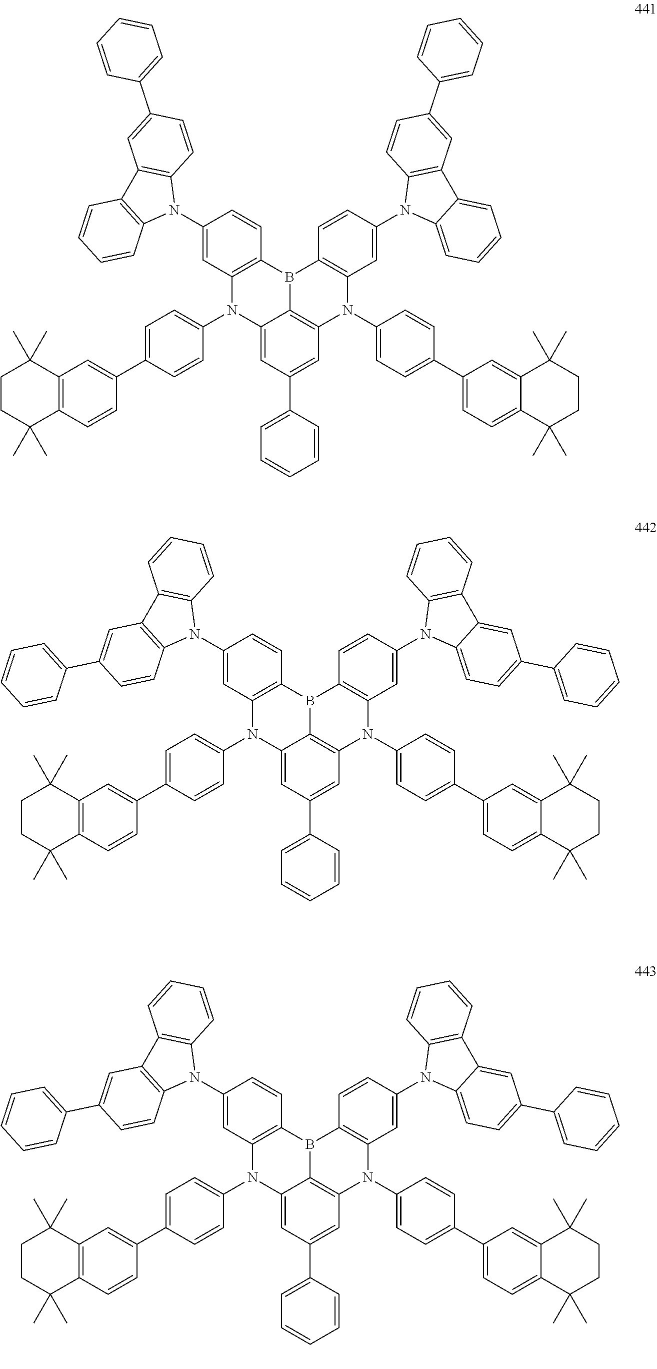

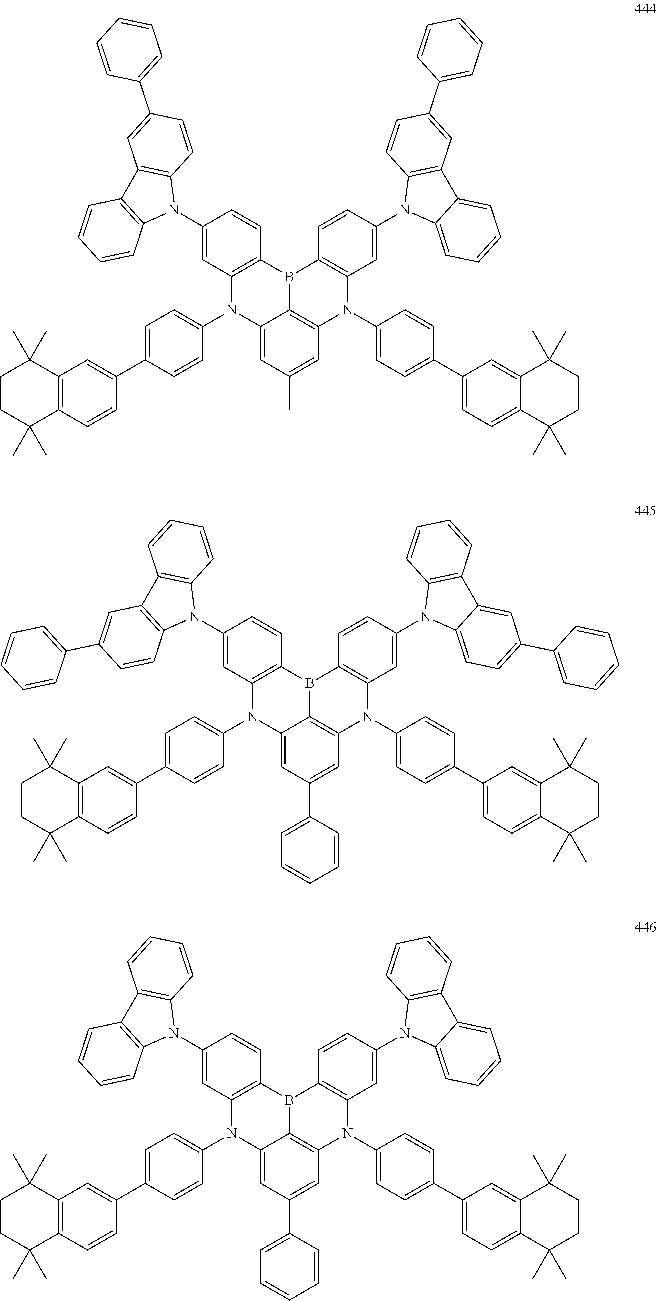

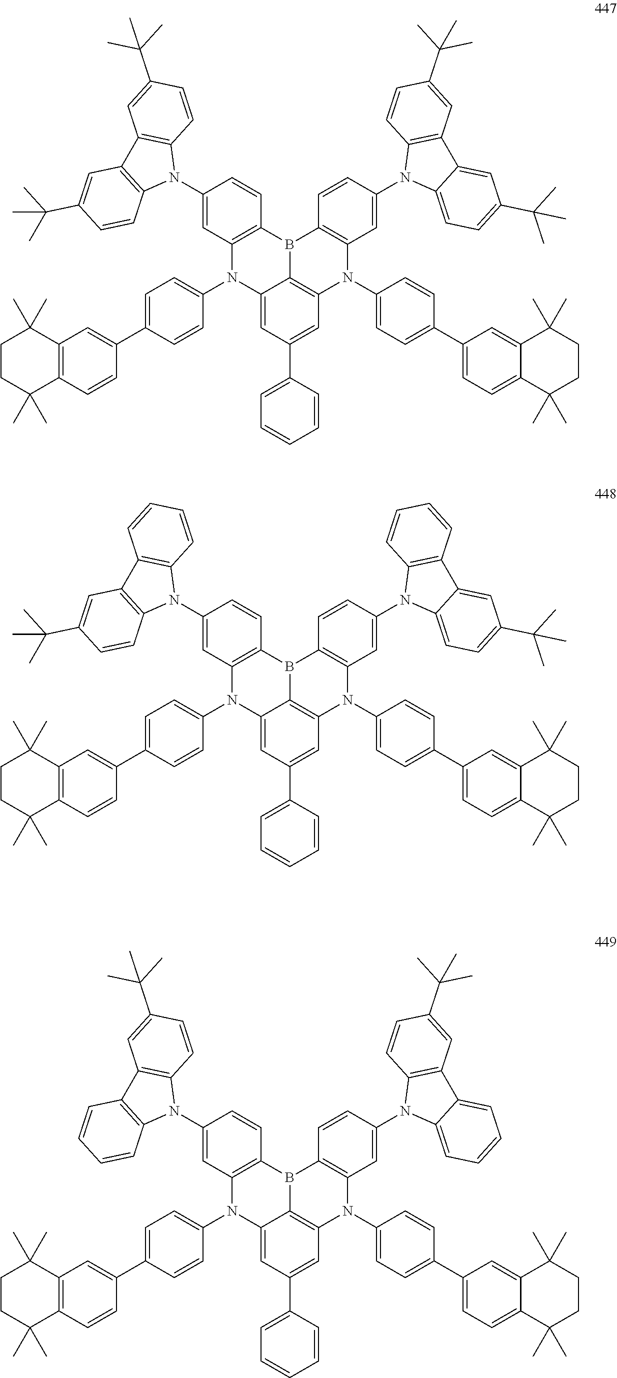

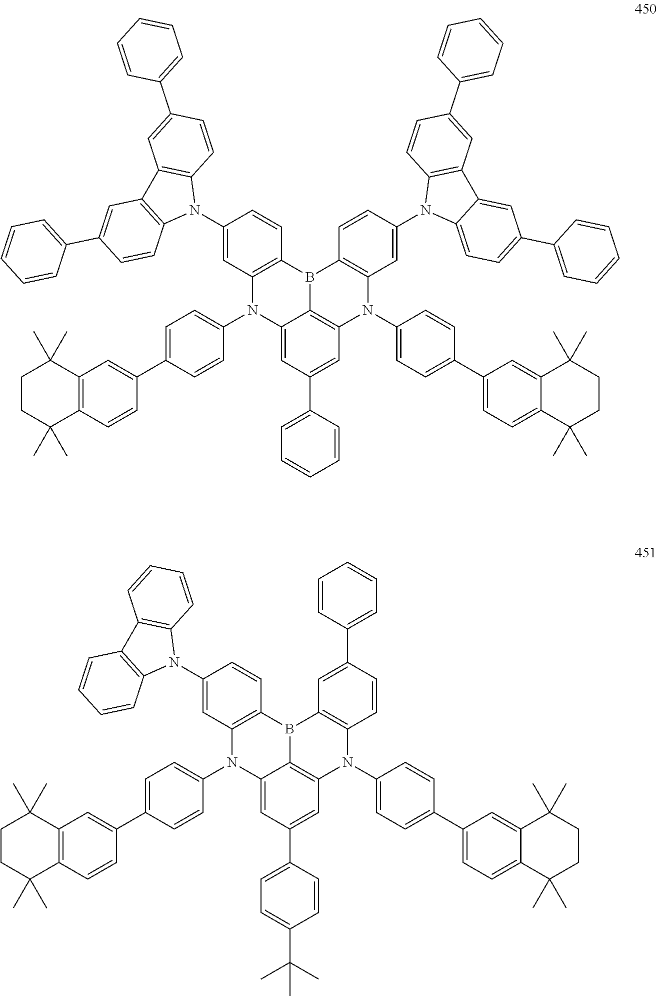 [SMILES: CC1(C)CCC(C)(C)c2cc(-c3ccc(N4c5cc(-n6c7ccc(-c8ccccc8)cc7c7cc(-c8ccccc8)ccc76)ccc5B5c6ccc(-n7c8ccc(-c9ccccc9)cc8c8cc(-c9ccccc9)ccc87)cc6N(c6ccc(-c7ccc8c(c7)C(C)(C)CCC8(C)C)cc6)c6cc(-c7ccccc7)cc4c65)cc3)ccc21 ; CC(C)(C)c1ccc(-c2cc3c4c(c2)N(c2ccc(-c5ccc6c(c5)C(C)(C)CCC6(C)C)cc2)c2cc(-n5c6ccccc6c6ccccc65)ccc2B4c2cc(-c4ccccc4)ccc2N3c2ccc(-c3ccc4c(c3)C(C)(C)CCC4(C)C)cc2)cc1]

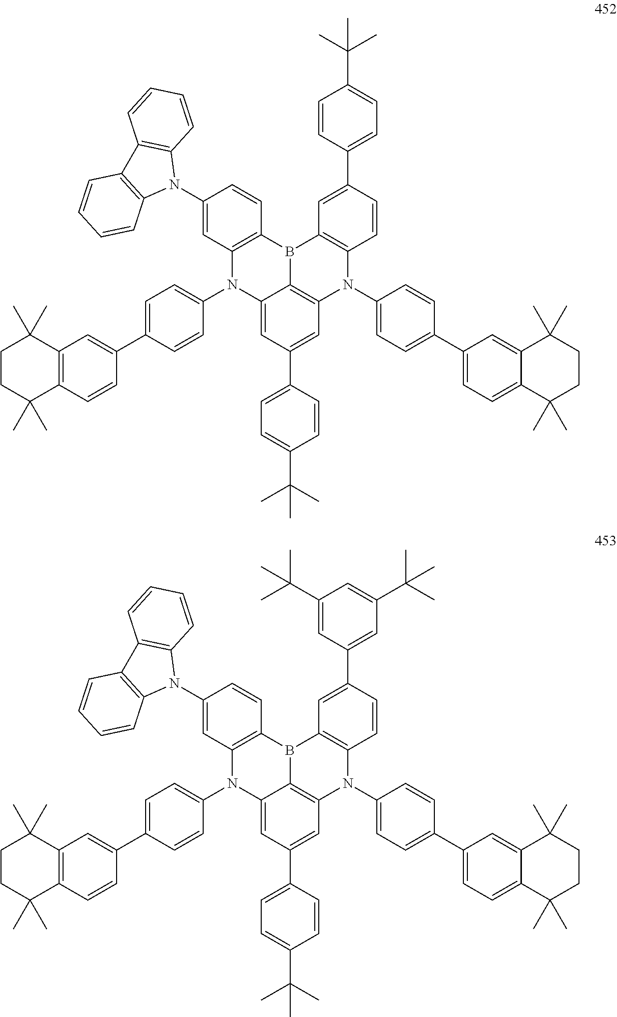

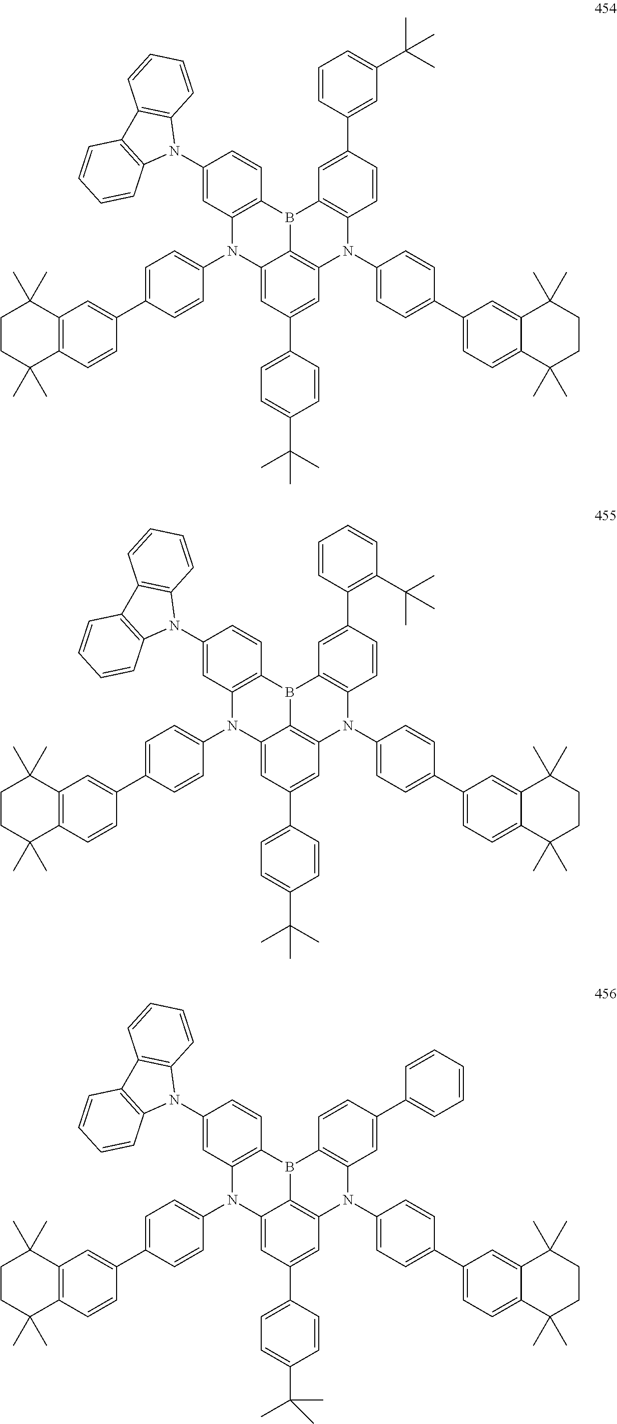

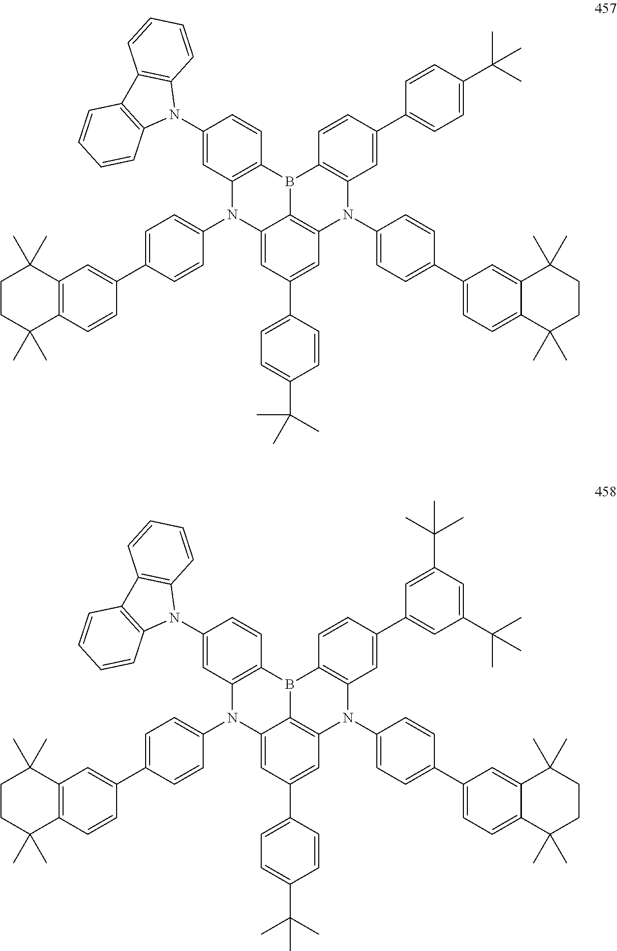 [SMILES: CC(C)(C)c1ccc(-c2ccc3c(c2)N(c2ccc(-c4ccc5c(c4)C(C)(C)CCC5(C)C)cc2)c2cc(-c4ccc(C(C)(C)C)cc4)cc4c2B3c2ccc(-n3c5ccccc5c5ccccc53)cc2N4c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccccc6c6ccccc65)ccc2B4c2ccc(-c4cc(C(C)(C)C)cc(C(C)(C)C)c4)cc2N3c2ccc(-c3ccc4c(c3)C(C)(C)CCC4(C)C)cc2)cc1]

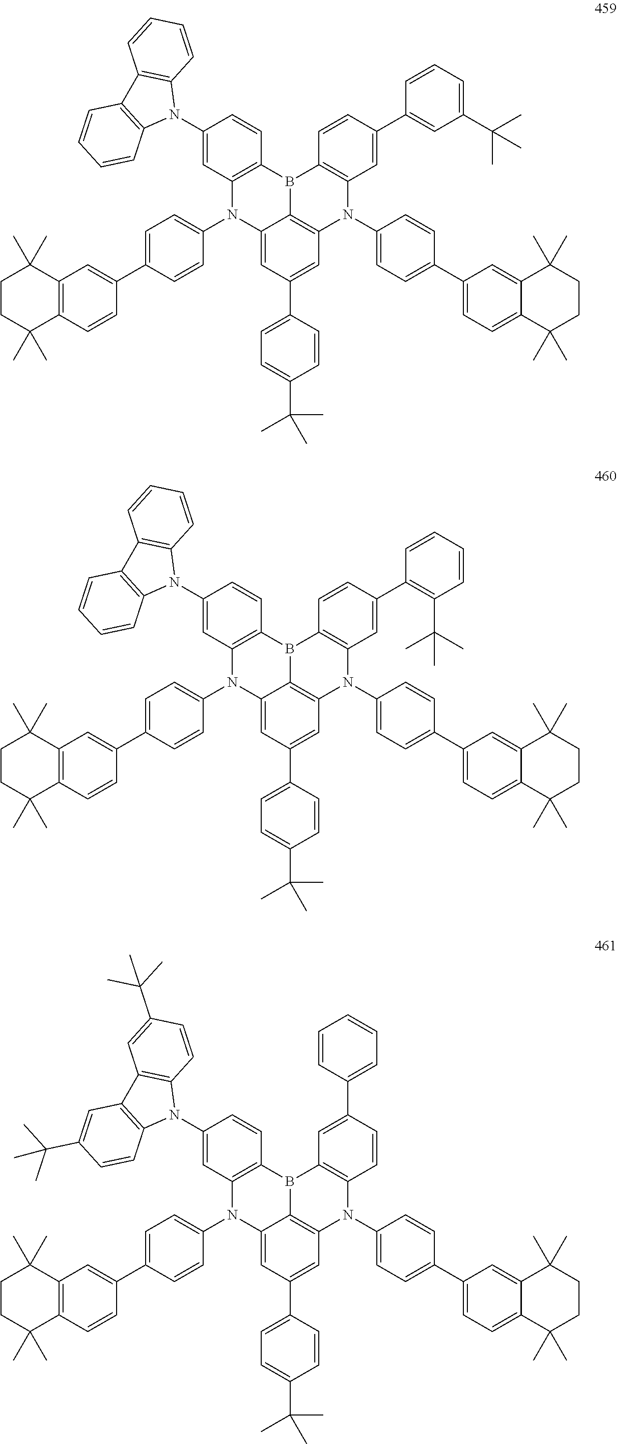

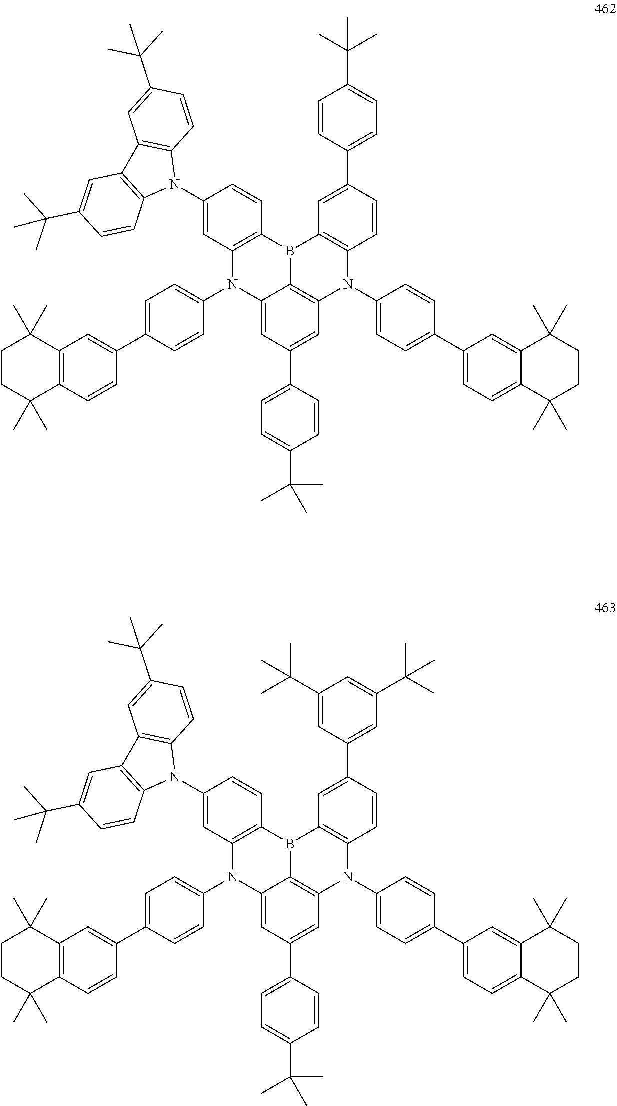 [SMILES: CC(C)(C)c1ccc(-c2ccc3c(c2)B2c4ccc(-n5c6ccc(C(C)(C)C)cc6c6cc(C(C)(C)C)ccc65)cc4N(c4ccc(-c5ccc6c(c5)C(C)(C)CCC6(C)C)cc4)c4cc(-c5ccc(C(C)(C)C)cc5)cc(c42)N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccc(C(C)(C)C)cc6c6cc(C(C)(C)C)ccc65)ccc2B4c2cc(-c4cc(C(C)(C)C)cc(C(C)(C)C)c4)ccc2N3c2ccc(-c3ccc4c(c3)C(C)(C)CCC4(C)C)cc2)cc1]

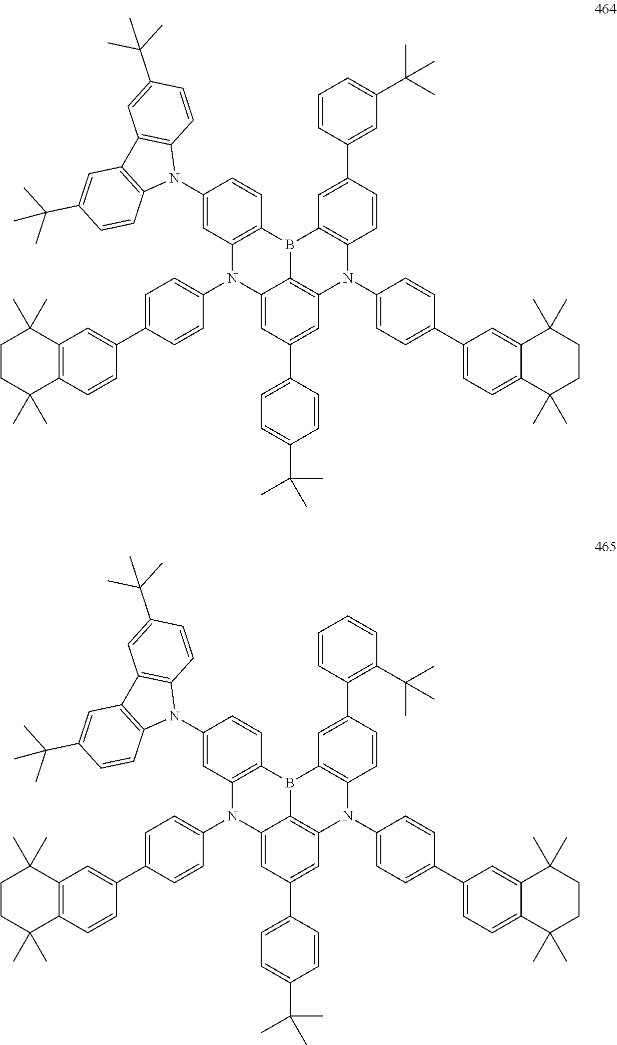 [SMILES: CC(C)(C)c1ccc(-c2cc3c4c(c2)N(c2ccc(-c5ccc6c(c5)C(C)(C)CCC6(C)C)cc2)c2cc(-n5c6ccc(C(C)(C)C)cc6c6cc(C(C)(C)C)ccc65)ccc2B4c2cc(-c4cccc(C(C)(C)C)c4)ccc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccc(C(C)(C)C)cc6c6cc(C(C)(C)C)ccc65)ccc2B4c2cc(-c4ccccc4C(C)(C)C)ccc2N3c2ccc(-c3ccc4c(c3)C(C)(C)CCC4(C)C)cc2)cc1]

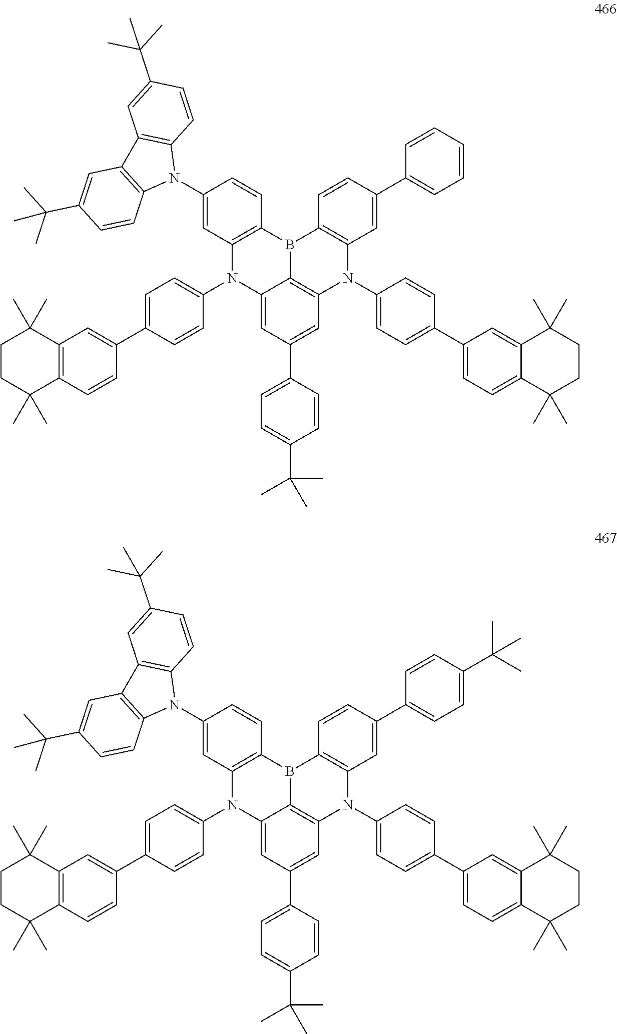 [SMILES: CC(C)(C)c1ccc(-c2cc3c4c(c2)N(c2ccc(-c5ccc6c(c5)C(C)(C)CCC6(C)C)cc2)c2cc(-n5c6ccc(C(C)(C)C)cc6c6cc(C(C)(C)C)ccc65)ccc2B4c2ccc(-c4ccccc4)cc2N3c2ccc(-c3ccc4c(c3)C(C)(C)CCC4(C)C)cc2)cc1 ; CC(C)(C)c1ccc(-c2ccc3c(c2)N(c2ccc(-c4ccc5c(c4)C(C)(C)CCC5(C)C)cc2)c2cc(-c4ccc(C(C)(C)C)cc4)cc4c2B3c2ccc(-n3c5ccc(C(C)(C)C)cc5c5cc(C(C)(C)C)ccc53)cc2N4c2ccc(-c3ccc4c(c3)C(C)(C)CCC4(C)C)cc2)cc1]

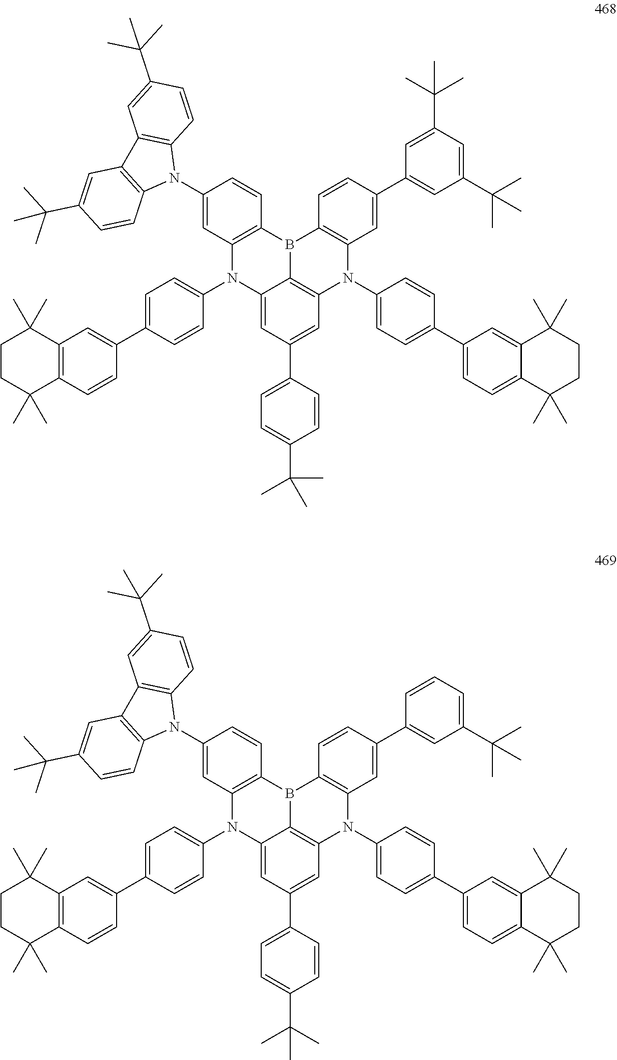 [SMILES: CC(C)(C)c1ccc(-c2cc3c4c(c2)N(c2ccc(-c5ccc6c(c5)C(C)(C)CCC6(C)C)cc2)c2cc(-n5c6ccc(C(C)(C)C)cc6c6cc(C(C)(C)C)ccc65)ccc2B4c2ccc(-c4cc(C(C)(C)C)cc(C(C)(C)C)c4)cc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccc(C(C)(C)C)cc6c6cc(C(C)(C)C)ccc65)ccc2B4c2ccc(-c4cccc(C(C)(C)C)c4)cc2N3c2ccc(-c3ccc4c(c3)C(C)(C)CCC4(C)C)cc2)cc1]

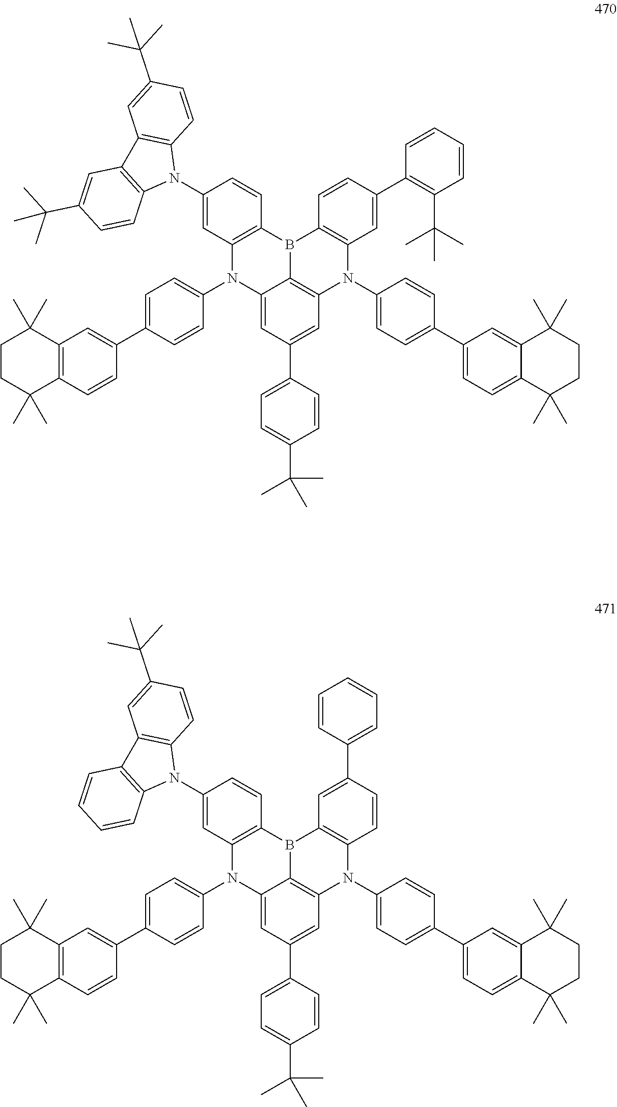 [SMILES: CC(C)(C)c1ccc(-c2cc3c4c(c2)N(c2ccc(-c5ccc6c(c5)C(C)(C)CCC6(C)C)cc2)c2cc(-n5c6ccc(C(C)(C)C)cc6c6cc(C(C)(C)C)ccc65)ccc2B4c2ccc(-c4ccccc4C(C)(C)C)cc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccccc6c6cc(C(C)(C)C)ccc65)ccc2B4c2cc(-c4ccccc4)ccc2N3c2ccc(-c3ccc4c(c3)C(C)(C)CCC4(C)C)cc2)cc1]

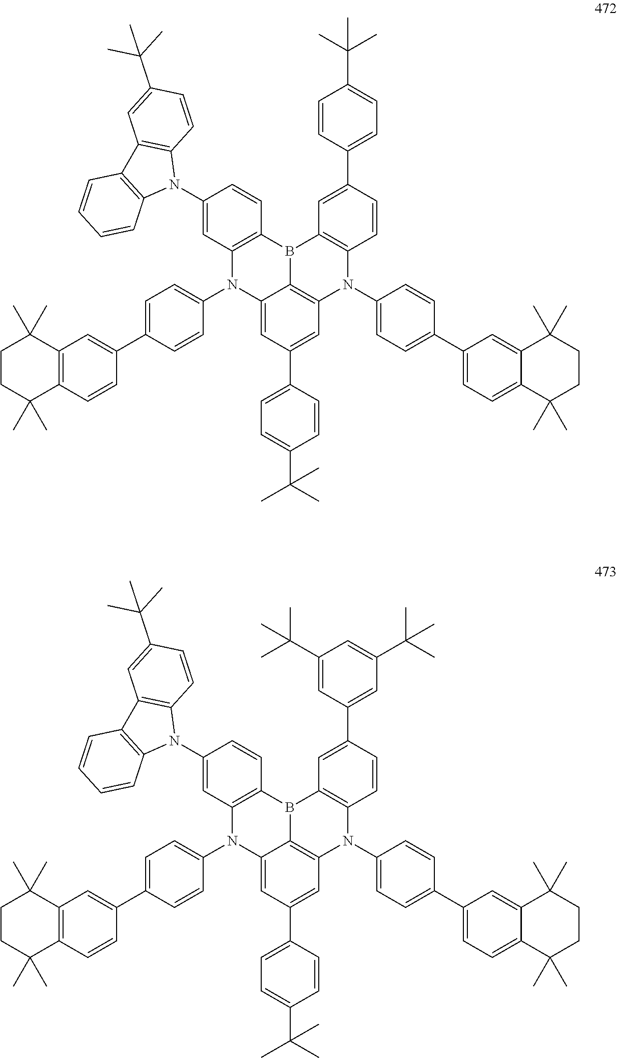 [SMILES: CC(C)(C)c1ccc(-c2ccc3c(c2)B2c4ccc(-n5c6ccccc6c6cc(C(C)(C)C)ccc65)cc4N(c4ccc(-c5ccc6c(c5)C(C)(C)CCC6(C)C)cc4)c4cc(-c5ccc(C(C)(C)C)cc5)cc(c42)N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccccc6c6cc(C(C)(C)C)ccc65)ccc2B4c2cc(-c4cc(C(C)(C)C)cc(C(C)(C)C)c4)ccc2N3c2ccc(-c3ccc4c(c3)C(C)(C)CCC4(C)C)cc2)cc1]

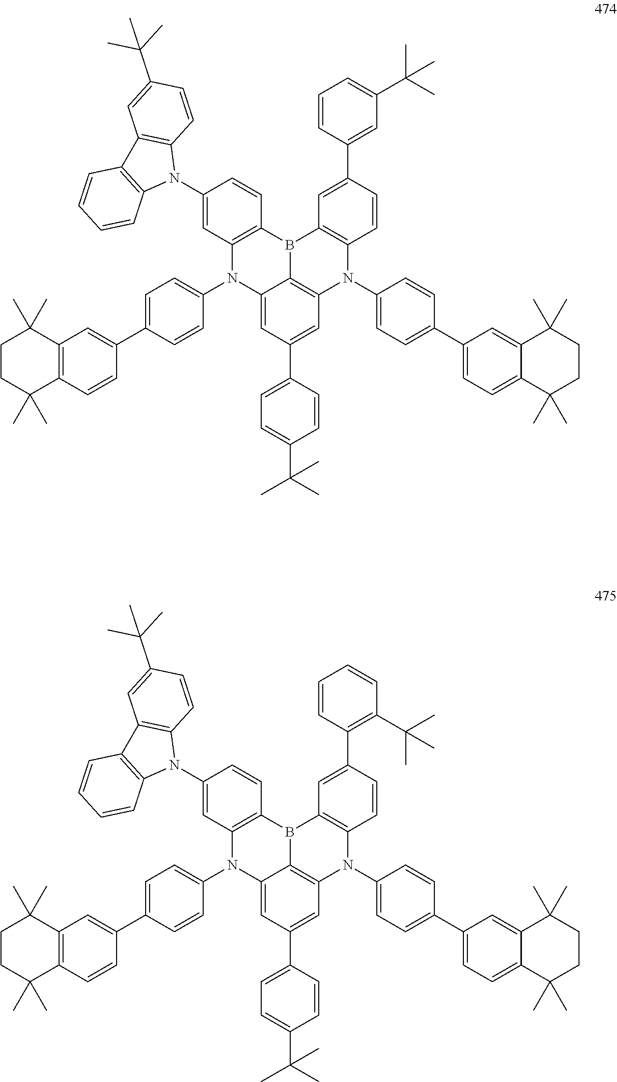 [SMILES: CC(C)(C)c1ccc(-c2cc3c4c(c2)N(c2ccc(-c5ccc6c(c5)C(C)(C)CCC6(C)C)cc2)c2cc(-n5c6ccccc6c6cc(C(C)(C)C)ccc65)ccc2B4c2cc(-c4cccc(C(C)(C)C)c4)ccc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccccc6c6cc(C(C)(C)C)ccc65)ccc2B4c2cc(-c4ccccc4C(C)(C)C)ccc2N3c2ccc(-c3ccc4c(c3)C(C)(C)CCC4(C)C)cc2)cc1]

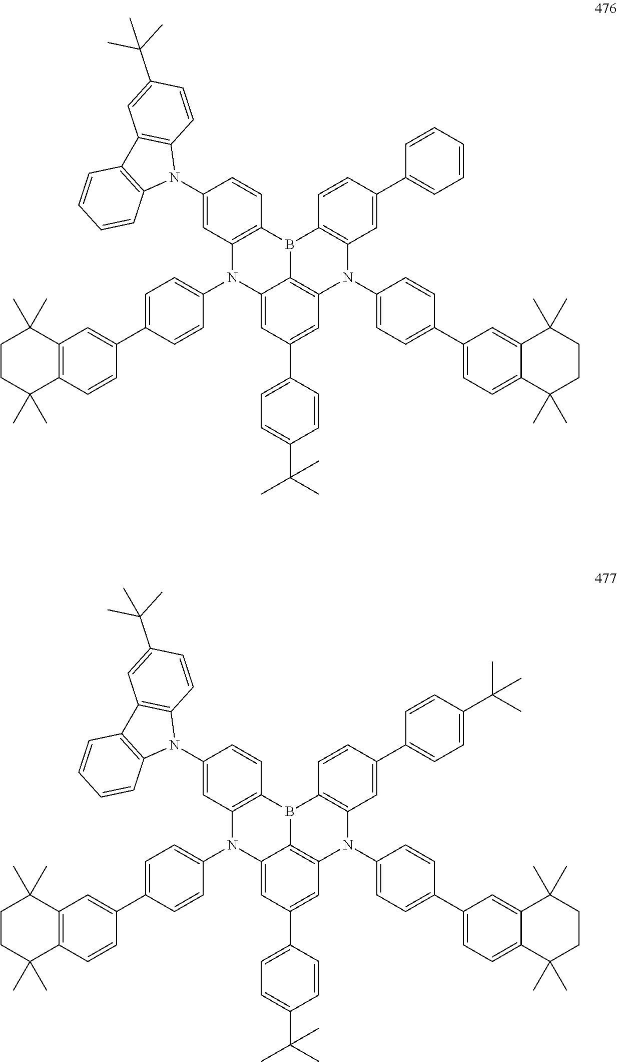 [SMILES: CC(C)(C)c1ccc(-c2cc3c4c(c2)N(c2ccc(-c5ccc6c(c5)C(C)(C)CCC6(C)C)cc2)c2cc(-n5c6ccccc6c6cc(C(C)(C)C)ccc65)ccc2B4c2ccc(-c4ccccc4)cc2N3c2ccc(-c3ccc4c(c3)C(C)(C)CCC4(C)C)cc2)cc1 ; CC(C)(C)c1ccc(-c2ccc3c(c2)N(c2ccc(-c4ccc5c(c4)C(C)(C)CCC5(C)C)cc2)c2cc(-c4ccc(C(C)(C)C)cc4)cc4c2B3c2ccc(-n3c5ccccc5c5cc(C(C)(C)C)ccc53)cc2N4c2ccc(-c3ccc4c(c3)C(C)(C)CCC4(C)C)cc2)cc1]

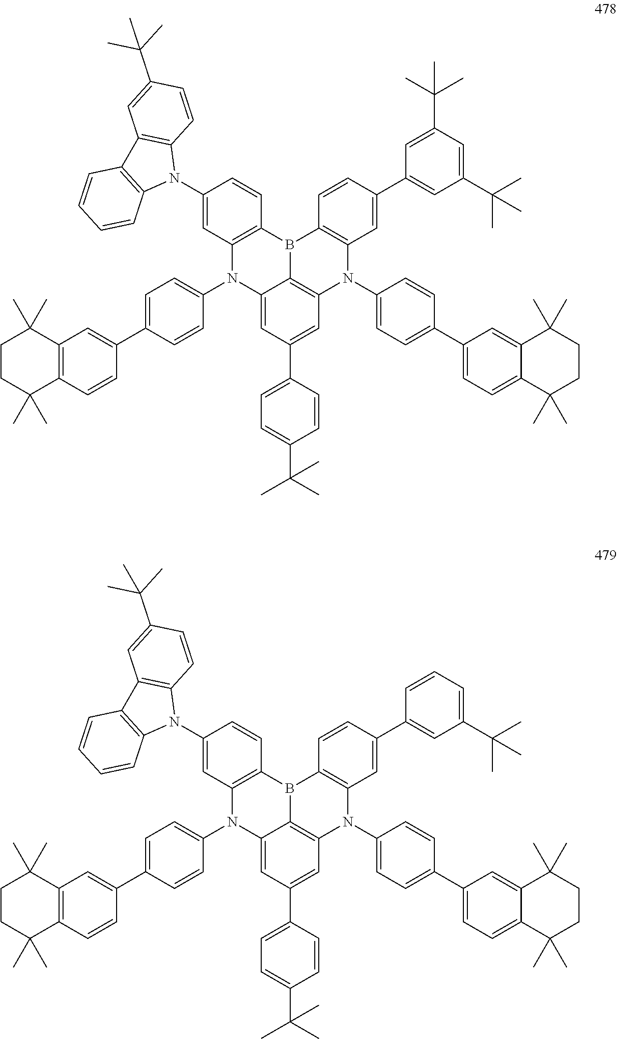 [SMILES: CC(C)(C)c1ccc(-c2cc3c4c(c2)N(c2ccc(-c5ccc6c(c5)C(C)(C)CCC6(C)C)cc2)c2cc(-n5c6ccccc6c6cc(C(C)(C)C)ccc65)ccc2B4c2ccc(-c4cc(C(C)(C)C)cc(C(C)(C)C)c4)cc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccccc6c6cc(C(C)(C)C)ccc65)ccc2B4c2ccc(-c4cccc(C(C)(C)C)c4)cc2N3c2ccc(-c3ccc4c(c3)C(C)(C)CCC4(C)C)cc2)cc1]

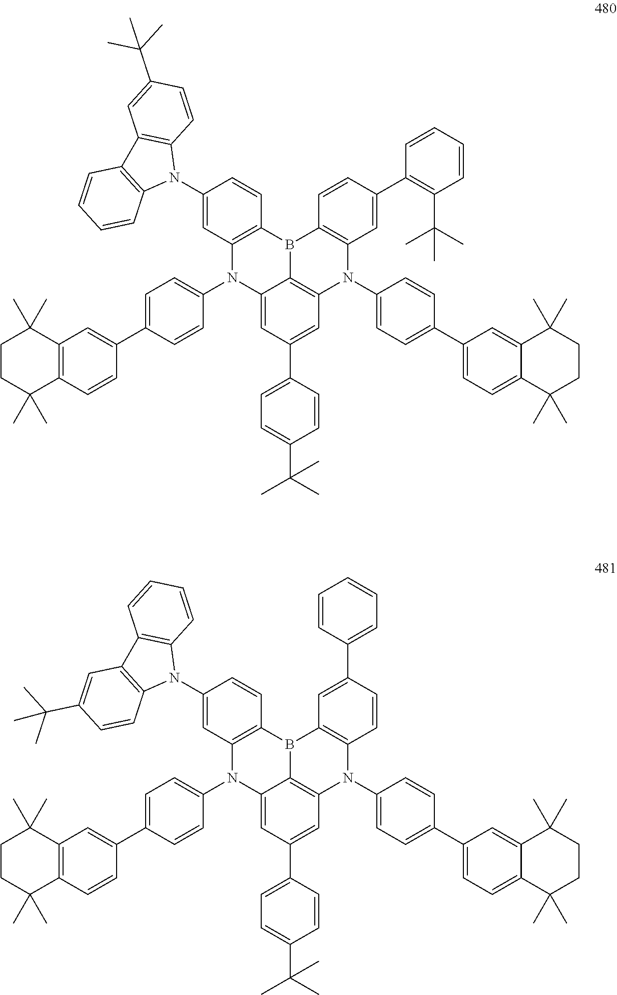 [SMILES: CC(C)(C)c1ccc(-c2cc3c4c(c2)N(c2ccc(-c5ccc6c(c5)C(C)(C)CCC6(C)C)cc2)c2cc(-n5c6ccccc6c6cc(C(C)(C)C)ccc65)ccc2B4c2ccc(-c4ccccc4C(C)(C)C)cc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccccc6c6cc(C(C)(C)C)ccc65)ccc2B4c2cc(-c4ccccc4)ccc2N3c2ccc(-c3ccc4c(c3)C(C)(C)CCC4(C)C)cc2)cc1]

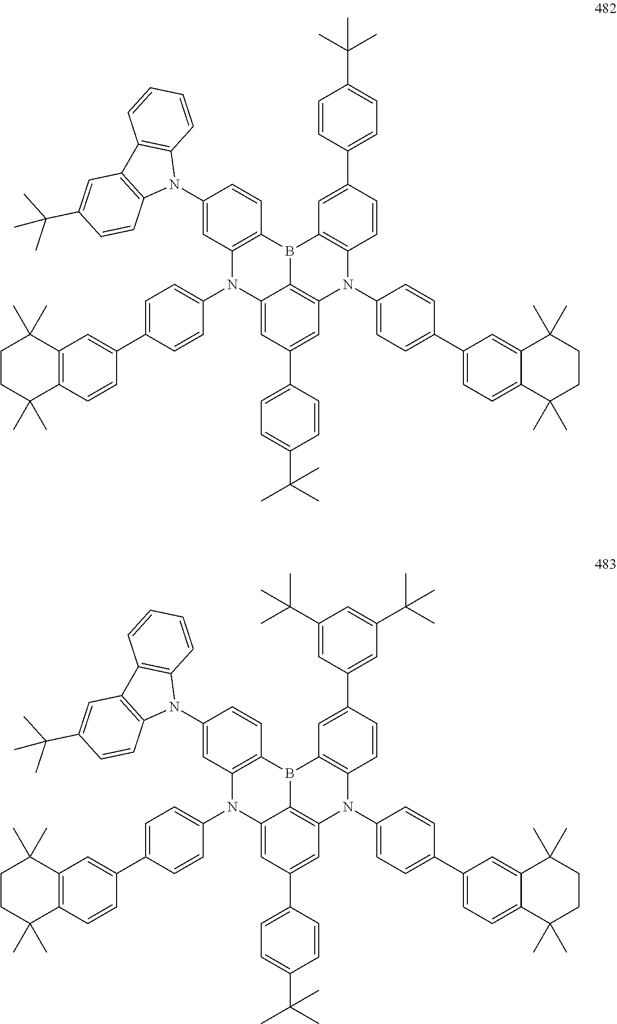 [SMILES: CC(C)(C)c1ccc(-c2ccc3c(c2)B2c4ccc(-n5c6ccccc6c6cc(C(C)(C)C)ccc65)cc4N(c4ccc(-c5ccc6c(c5)C(C)(C)CCC6(C)C)cc4)c4cc(-c5ccc(C(C)(C)C)cc5)cc(c42)N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccccc6c6cc(C(C)(C)C)ccc65)ccc2B4c2cc(-c4cc(C(C)(C)C)cc(C(C)(C)C)c4)ccc2N3c2ccc(-c3ccc4c(c3)C(C)(C)CCC4(C)C)cc2)cc1]

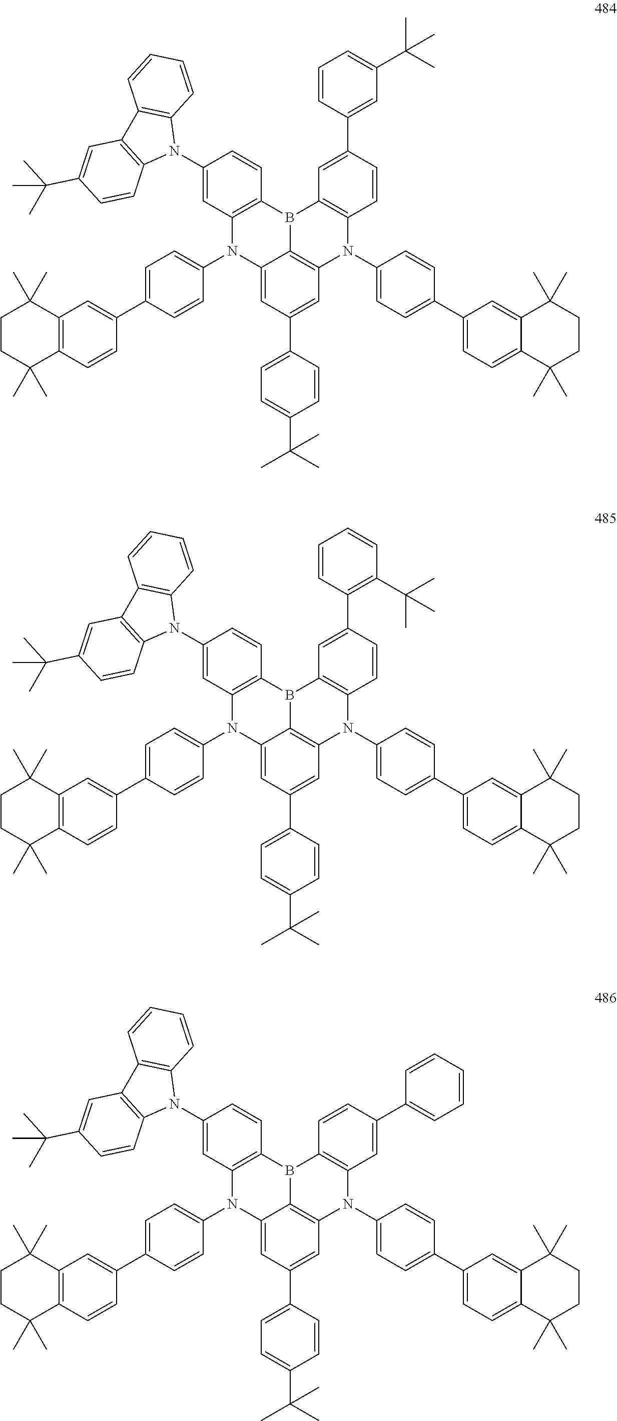

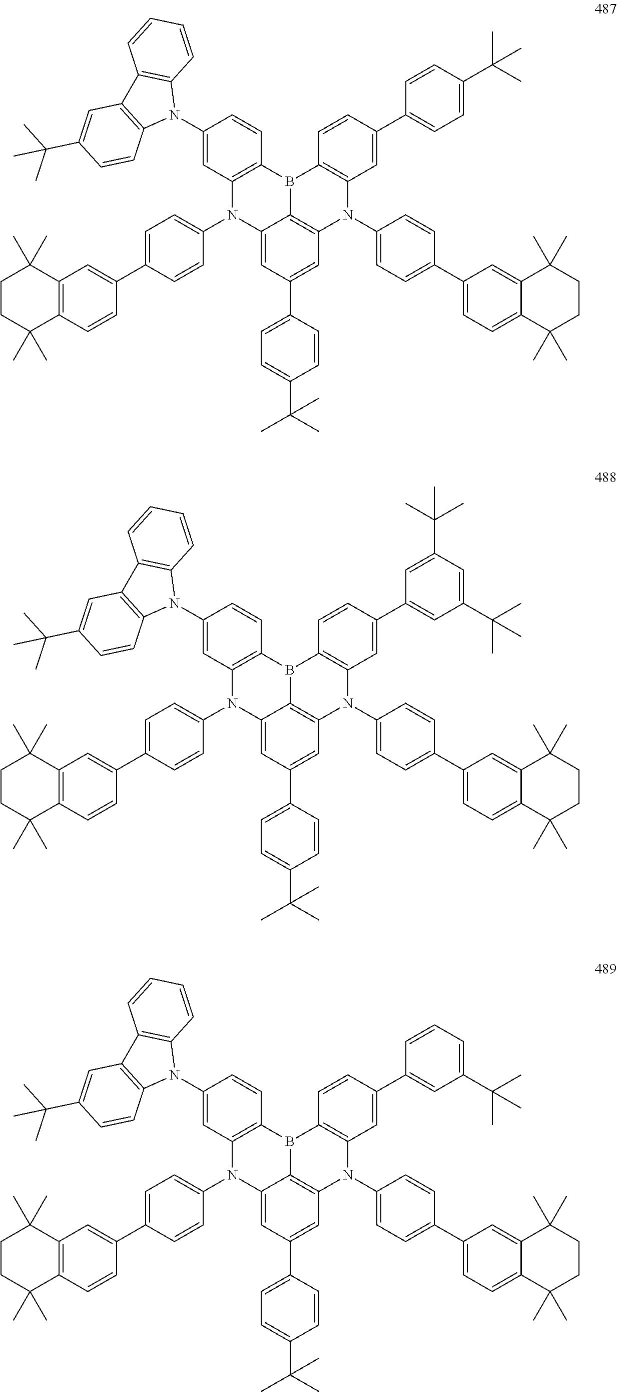

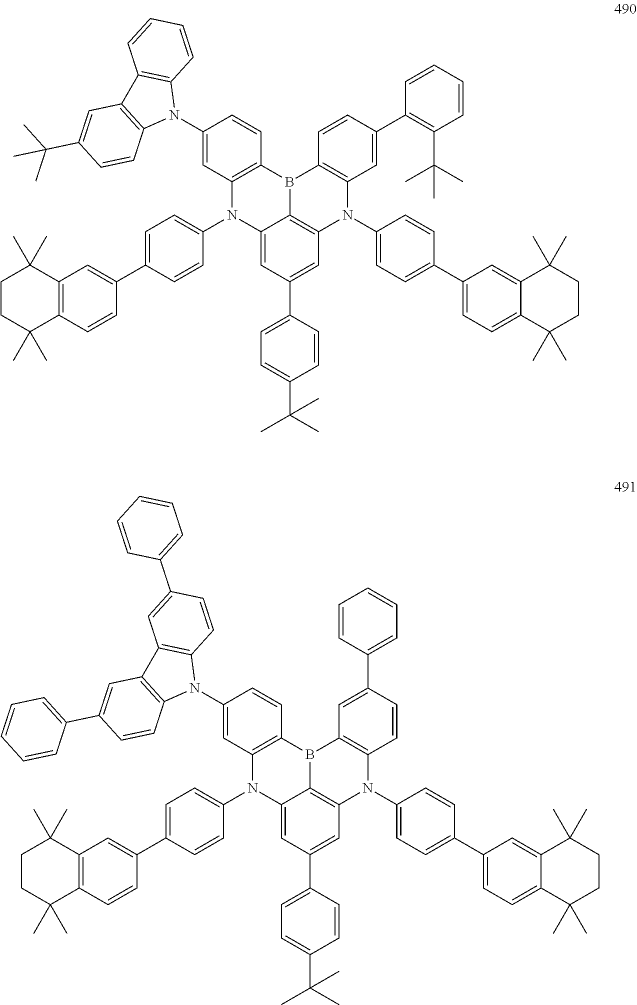 [SMILES: CC(C)(C)c1ccc(-c2cc3c4c(c2)N(c2ccc(-c5ccc6c(c5)C(C)(C)CCC6(C)C)cc2)c2cc(-n5c6ccccc6c6cc(C(C)(C)C)ccc65)ccc2B4c2ccc(-c4ccccc4C(C)(C)C)cc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2cc(-c4ccccc4)ccc2N3c2ccc(-c3ccc4c(c3)C(C)(C)CCC4(C)C)cc2)cc1]

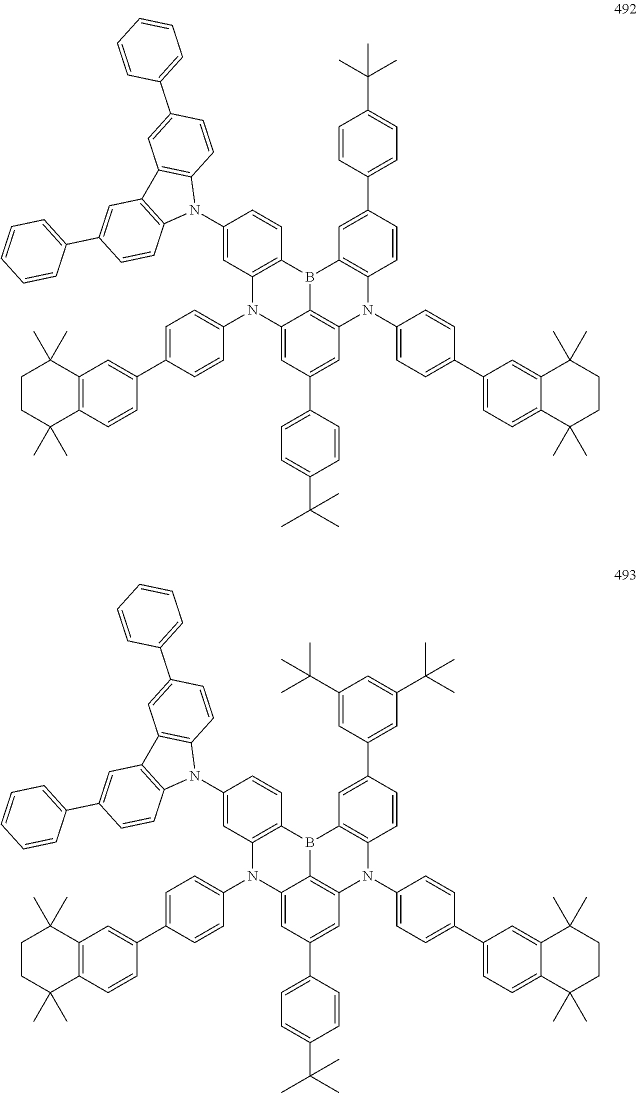 [SMILES: CC(C)(C)c1ccc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CCC6(C)C)cc4)c4cc(-c5ccc(C(C)(C)C)cc5)cc(c42)N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2cc(-c4cc(C(C)(C)C)cc(C(C)(C)C)c4)ccc2N3c2ccc(-c3ccc4c(c3)C(C)(C)CCC4(C)C)cc2)cc1]

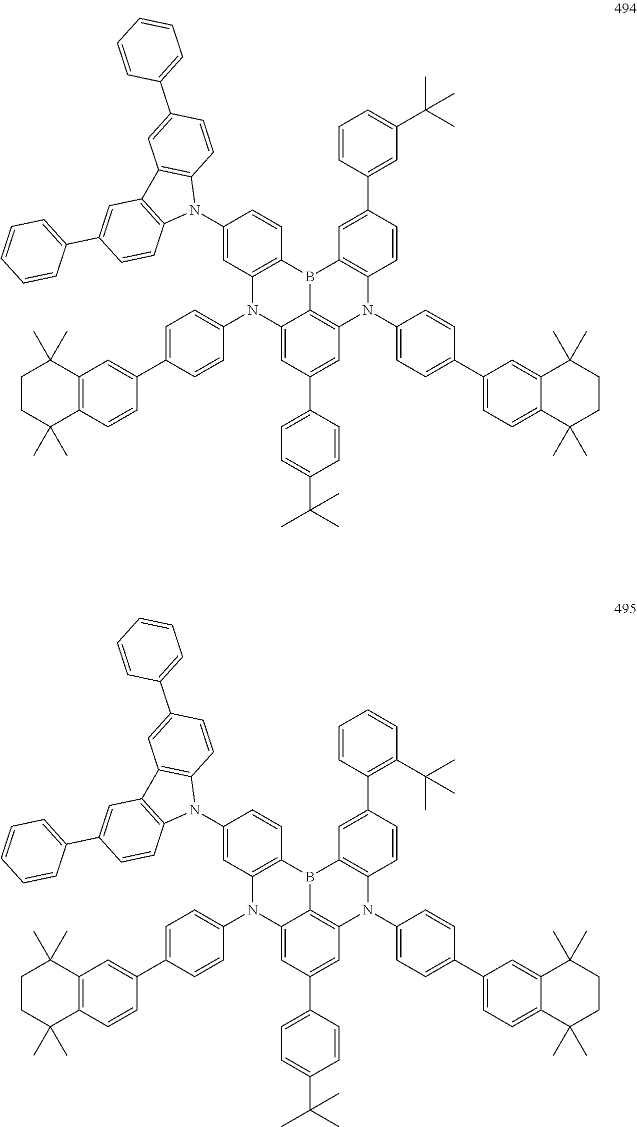 [SMILES: CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2cc(-c4cccc(C(C)(C)C)c4)ccc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2cc(-c4ccccc4C(C)(C)C)ccc2N3c2ccc(-c3ccc4c(c3)C(C)(C)CCC4(C)C)cc2)cc1]

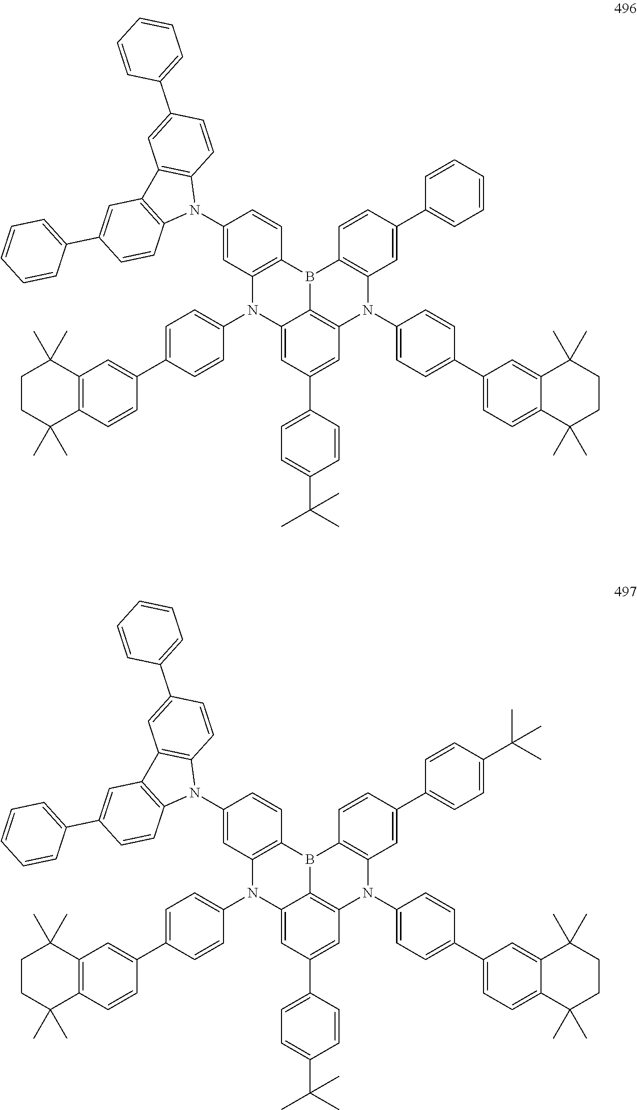 [SMILES: CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2ccc(-c4ccccc4)cc2N3c2ccc(-c3ccc4c(c3)C(C)(C)CCC4(C)C)cc2)cc1 ; CC(C)(C)c1ccc(-c2ccc3c(c2)N(c2ccc(-c4ccc5c(c4)C(C)(C)CCC5(C)C)cc2)c2cc(-c4ccc(C(C)(C)C)cc4)cc4c2B3c2ccc(-n3c5ccc(-c6ccccc6)cc5c5cc(-c6ccccc6)ccc53)cc2N4c2ccc(-c3ccc4c(c3)C(C)(C)CCC4(C)C)cc2)cc1]

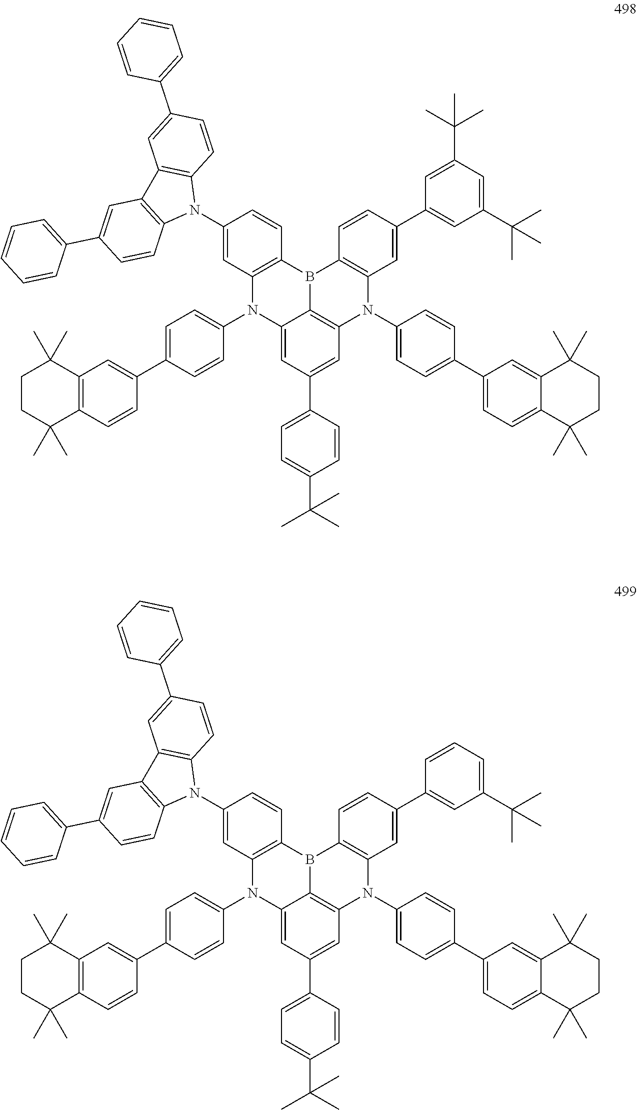 [SMILES: CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2ccc(-c4cc(C(C)(C)C)cc(C(C)(C)C)c4)cc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2ccc(-c4cccc(C(C)(C)C)c4)cc2N3c2ccc(-c3ccc4c(c3)C(C)(C)CCC4(C)C)cc2)cc1]

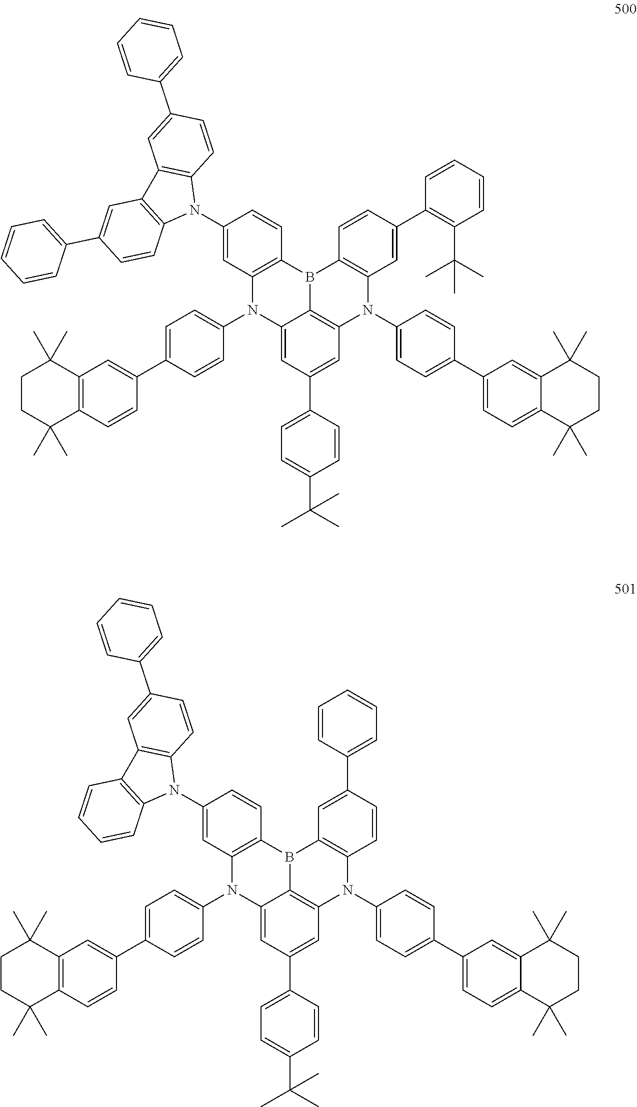 [SMILES: CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2ccc(-c4ccccc4C(C)(C)C)cc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccccc6c6cc(-c7ccccc7)ccc65)ccc2B4c2cc(-c4ccccc4)ccc2N3c2ccc(-c3ccc4c(c3)C(C)(C)CCC4(C)C)cc2)cc1]

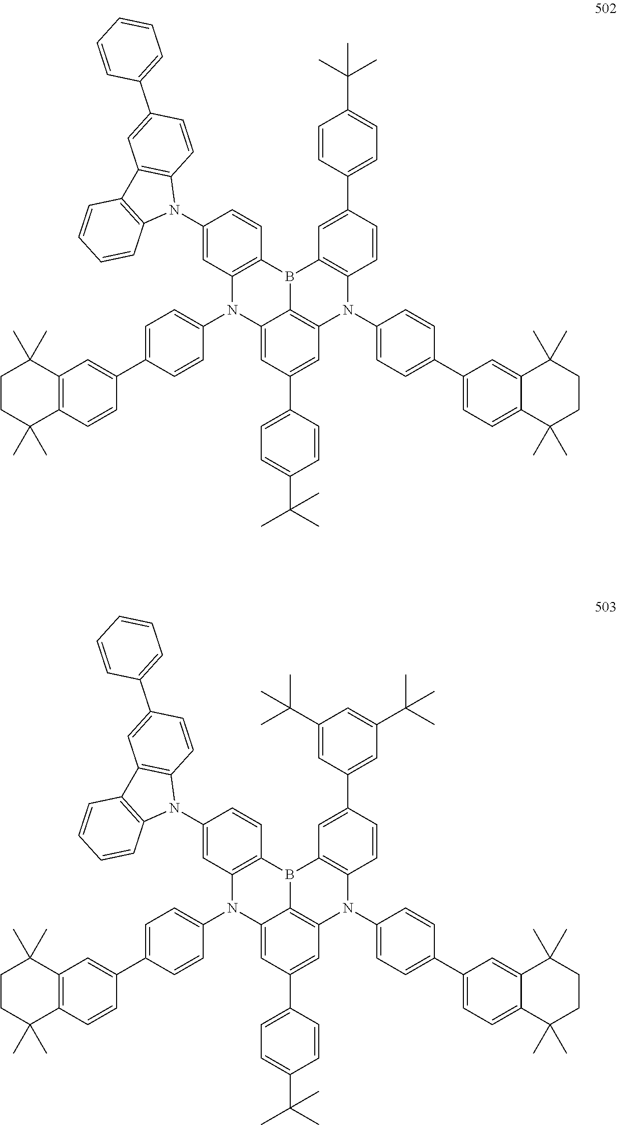 [SMILES: CC(C)(C)c1ccc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-c5ccc(C(C)(C)C)cc5)cc(c42)N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccccc6c6cc(-c7ccccc7)ccc65)ccc2B4c2cc(-c4cc(C(C)(C)C)cc(C(C)(C)C)c4)ccc2N3c2ccc(-c3ccc4c(c3)C(C)(C)CCC4(C)C)cc2)cc1]

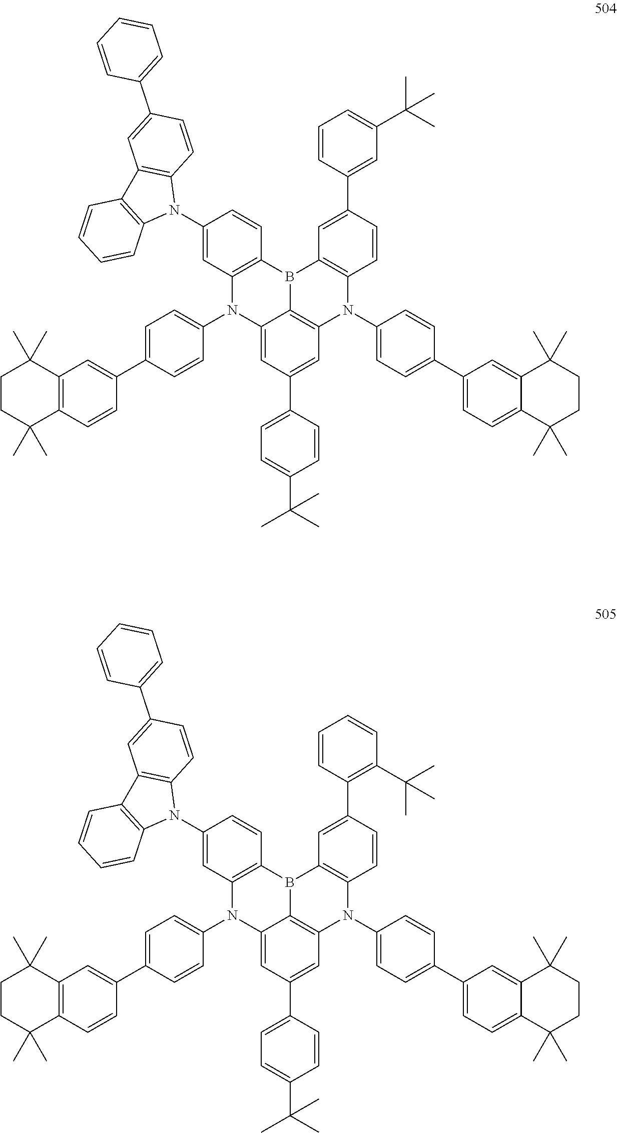 [SMILES: CC(C)(C)c1ccc(-c2cc3c4c(c2)N(c2ccc(-c5ccc6c(c5)C(C)(C)CCC6(C)C)cc2)c2cc(-n5c6ccccc6c6cc(-c7ccccc7)ccc65)ccc2B4c2cc(-c4cccc(C(C)(C)C)c4)ccc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccccc6c6cc(-c7ccccc7)ccc65)ccc2B4c2cc(-c4ccccc4C(C)(C)C)ccc2N3c2ccc(-c3ccc4c(c3)C(C)(C)CCC4(C)C)cc2)cc1]

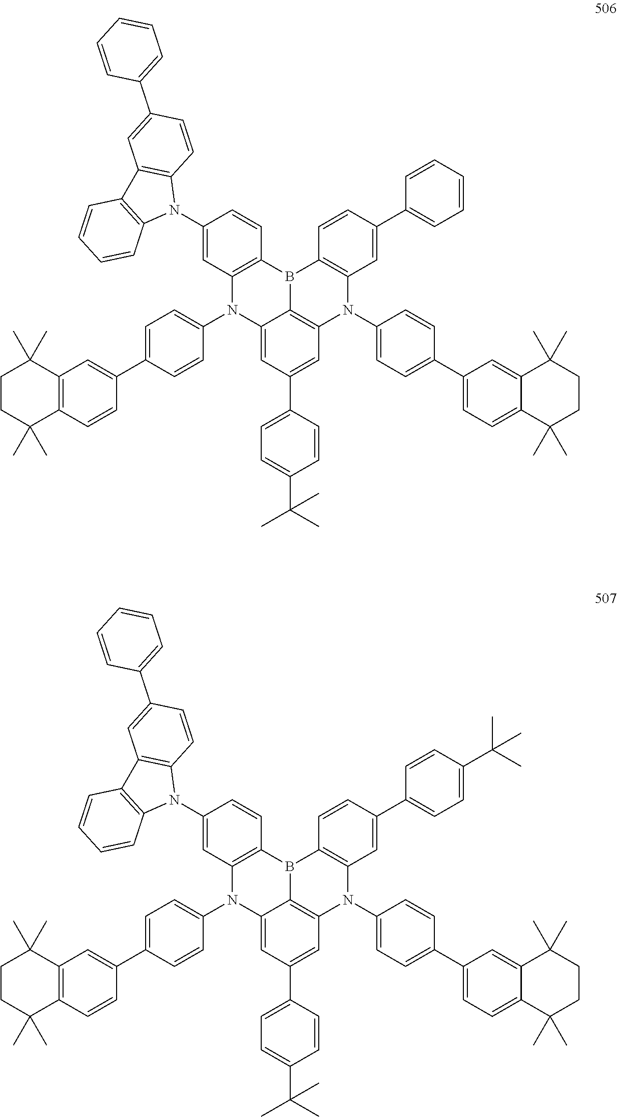 [SMILES: CC(C)(C)c1ccc(-c2cc3c4c(c2)N(c2ccc(-c5ccc6c(c5)C(C)(C)CCC6(C)C)cc2)c2cc(-n5c6ccccc6c6cc(-c7ccccc7)ccc65)ccc2B4c2ccc(-c4ccccc4)cc2N3c2ccc(-c3ccc4c(c3)C(C)(C)CCC4(C)C)cc2)cc1 ; CC(C)(C)c1ccc(-c2ccc3c(c2)N(c2ccc(-c4ccc5c(c4)C(C)(C)CCC5(C)C)cc2)c2cc(-c4ccc(C(C)(C)C)cc4)cc4c2B3c2ccc(-n3c5ccccc5c5cc(-c6ccccc6)ccc53)cc2N4c2ccc(-c3ccc4c(c3)C(C)(C)CCC4(C)C)cc2)cc1]

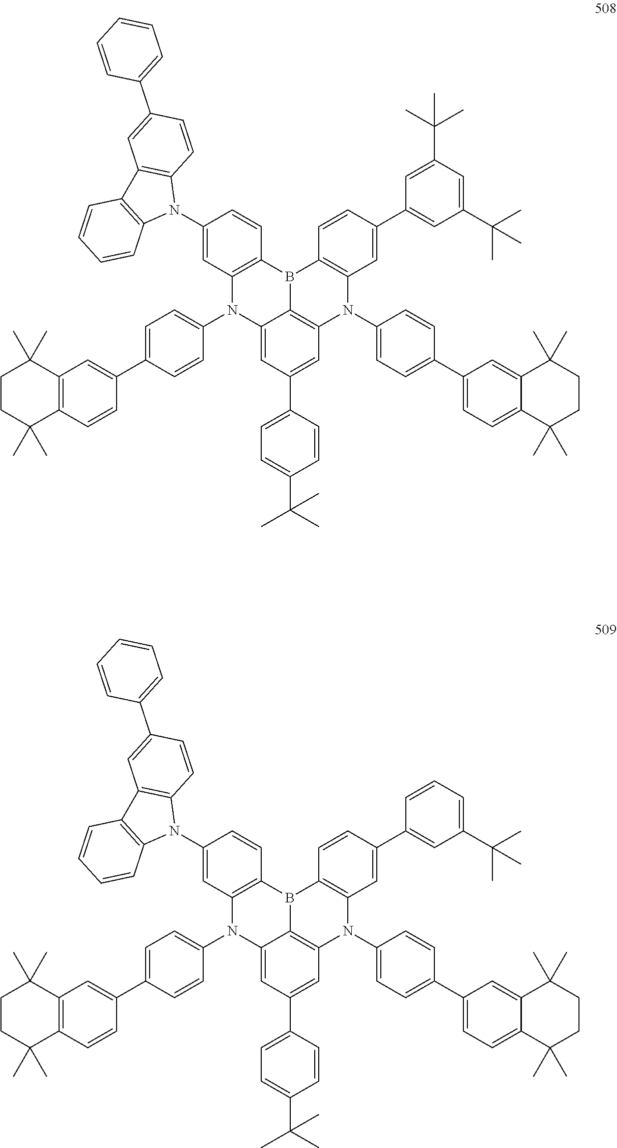 [SMILES: CC(C)(C)c1ccc(-c2cc3c4c(c2)N(c2ccc(-c5ccc6c(c5)C(C)(C)CCC6(C)C)cc2)c2cc(-n5c6ccccc6c6cc(-c7ccccc7)ccc65)ccc2B4c2ccc(-c4cc(C(C)(C)C)cc(C(C)(C)C)c4)cc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccccc6c6cc(-c7ccccc7)ccc65)ccc2B4c2ccc(-c4cccc(C(C)(C)C)c4)cc2N3c2ccc(-c3ccc4c(c3)C(C)(C)CCC4(C)C)cc2)cc1]

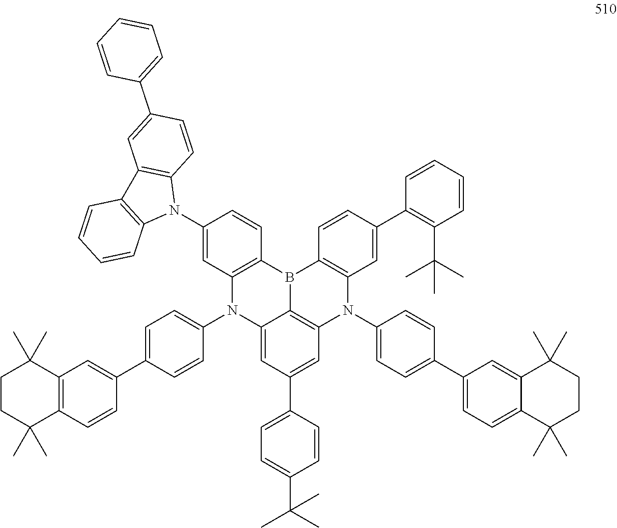 [SMILES: CC(C)(C)c1ccc(-c2cc3c4c(c2)N(c2ccc(-c5ccc6c(c5)C(C)(C)CCC6(C)C)cc2)c2cc(-n5c6ccccc6c6cc(-c7ccccc7)ccc65)ccc2B4c2ccc(-c4ccccc4C(C)(C)C)cc2N3c2ccc(-c3ccc4c(c3)C(C)(C)CCC4(C)C)cc2)cc1]

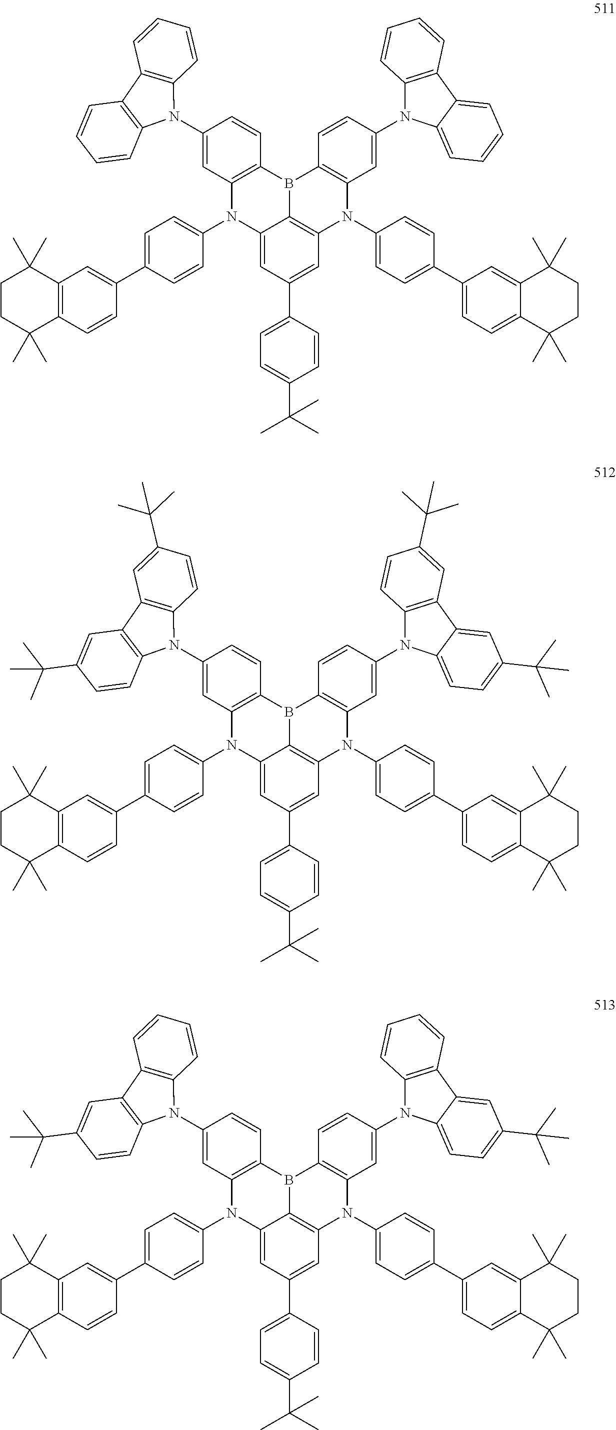

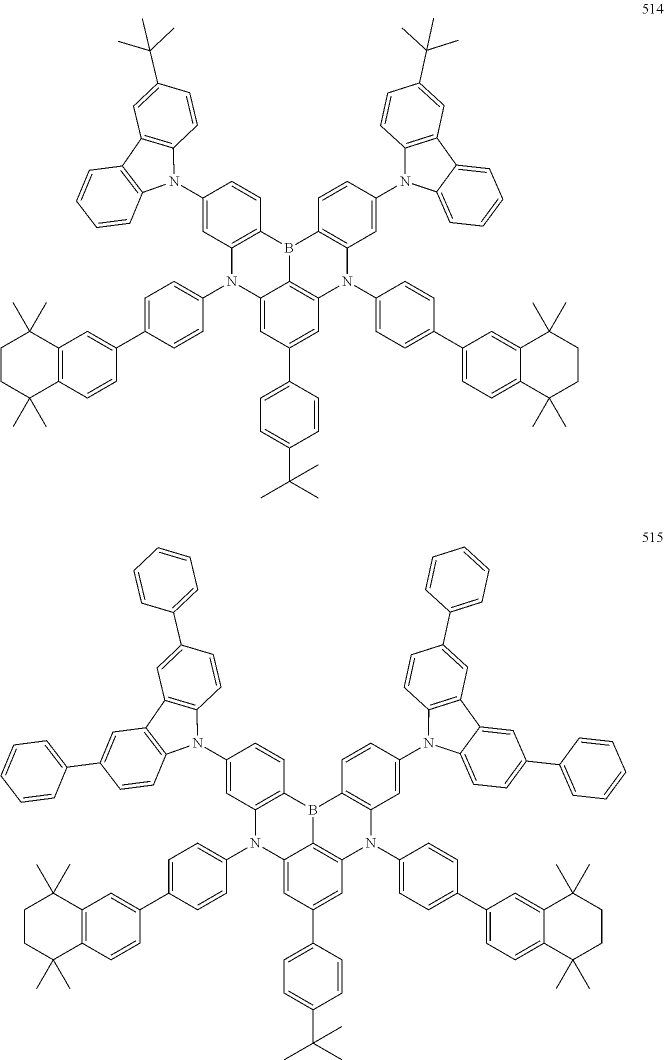 [SMILES: CC(C)(C)c1ccc(-c2cc3c4c(c2)N(c2ccc(-c5ccc6c(c5)C(C)(C)CCC6(C)C)cc2)c2cc(-n5c6ccccc6c6cc(C(C)(C)C)ccc65)ccc2B4c2ccc(-n4c5ccccc5c5cc(C(C)(C)C)ccc54)cc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2ccc(-n4c5ccc(-c6ccccc6)cc5c5cc(-c6ccccc6)ccc54)cc2N3c2ccc(-c3ccc4c(c3)C(C)(C)CCC4(C)C)cc2)cc1]

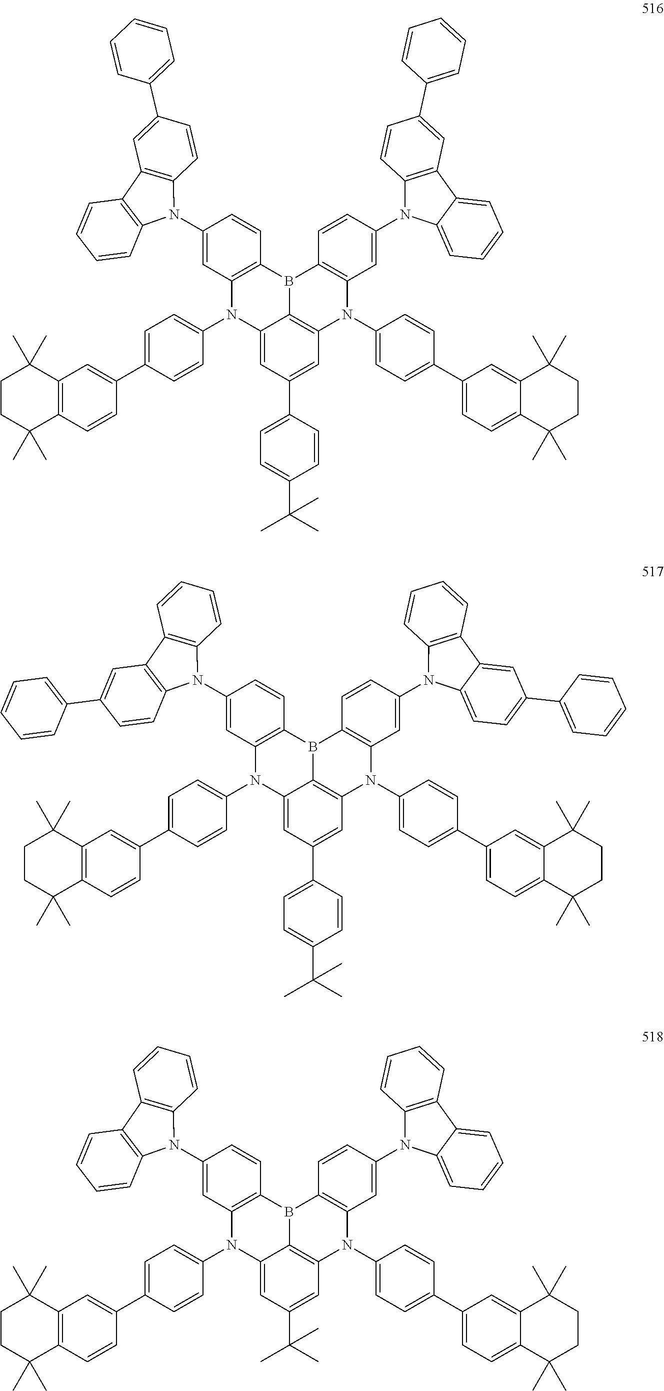

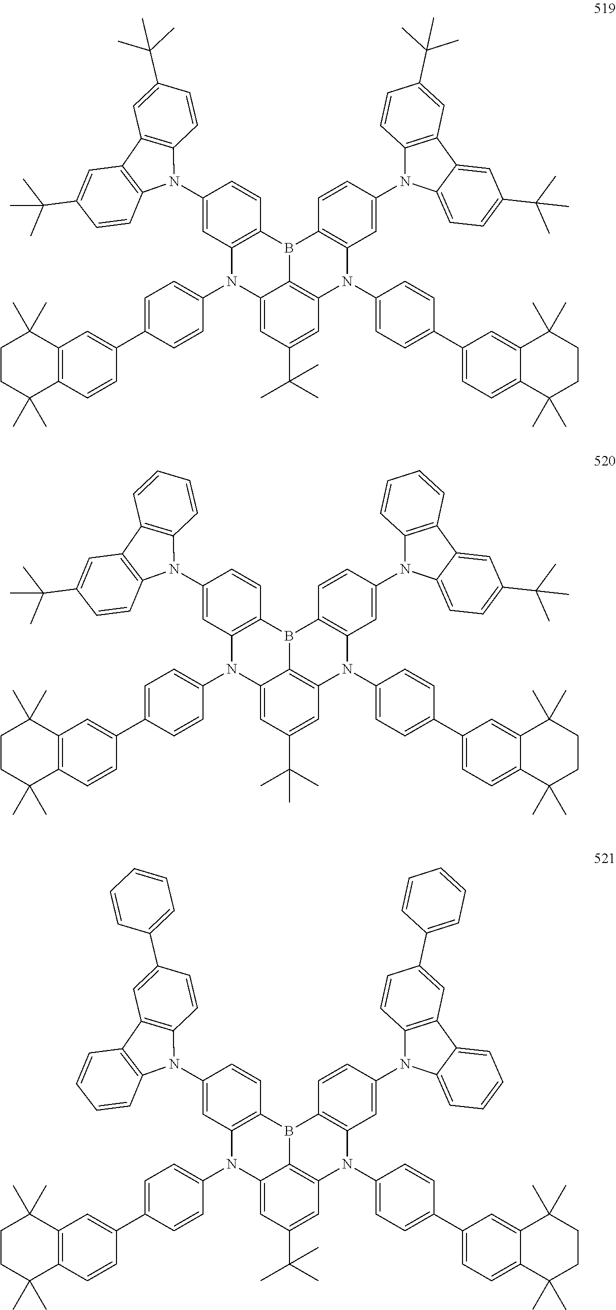

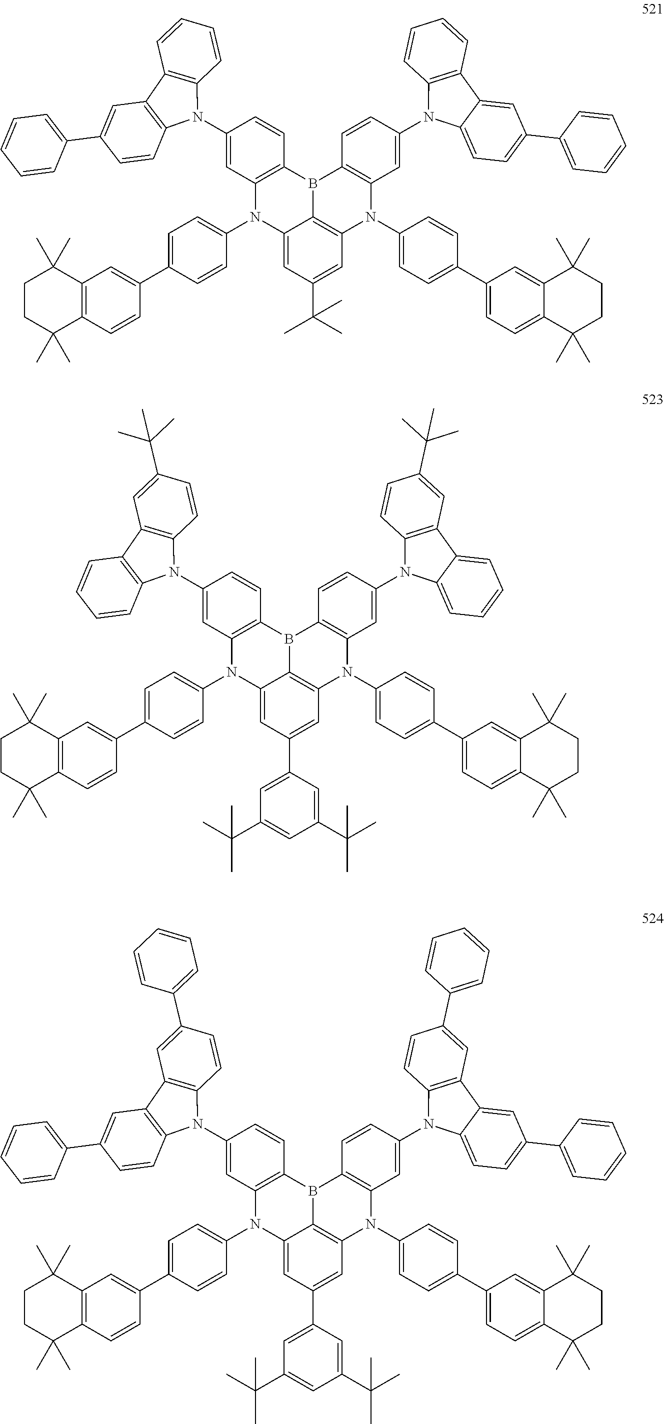

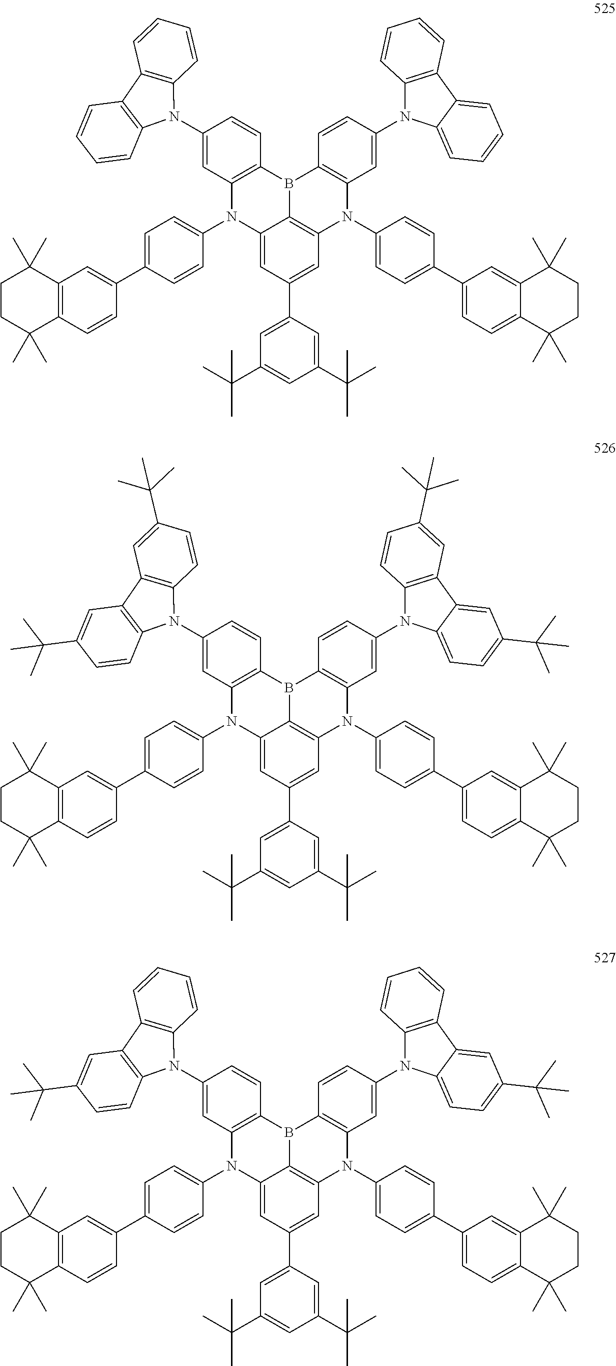

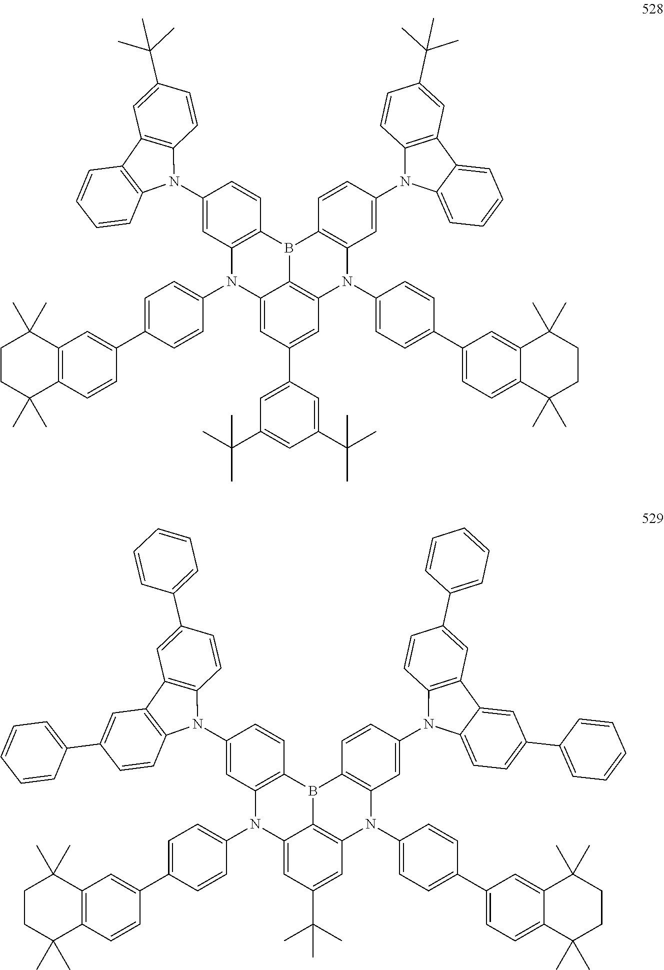 [SMILES: CC(C)(C)c1cc(-c2cc3c4c(c2)N(c2ccc(-c5ccc6c(c5)C(C)(C)CCC6(C)C)cc2)c2cc(-n5c6ccccc6c6cc(C(C)(C)C)ccc65)ccc2B4c2ccc(-n4c5ccccc5c5cc(C(C)(C)C)ccc54)cc2N3c2ccc(-c3ccc4c(c3)C(C)(C)CCC4(C)C)cc2)cc(C(C)(C)C)c1 ; CC(C)(C)c1cc2c3c(c1)N(c1ccc(-c4ccc5c(c4)C(C)(C)CCC5(C)C)cc1)c1cc(-n4c5ccc(-c6ccccc6)cc5c5cc(-c6ccccc6)ccc54)ccc1B3c1ccc(-n3c4ccc(-c5ccccc5)cc4c4cc(-c5ccccc5)ccc43)cc1N2c1ccc(-c2ccc3c(c2)C(C)(C)CCC3(C)C)cc1]

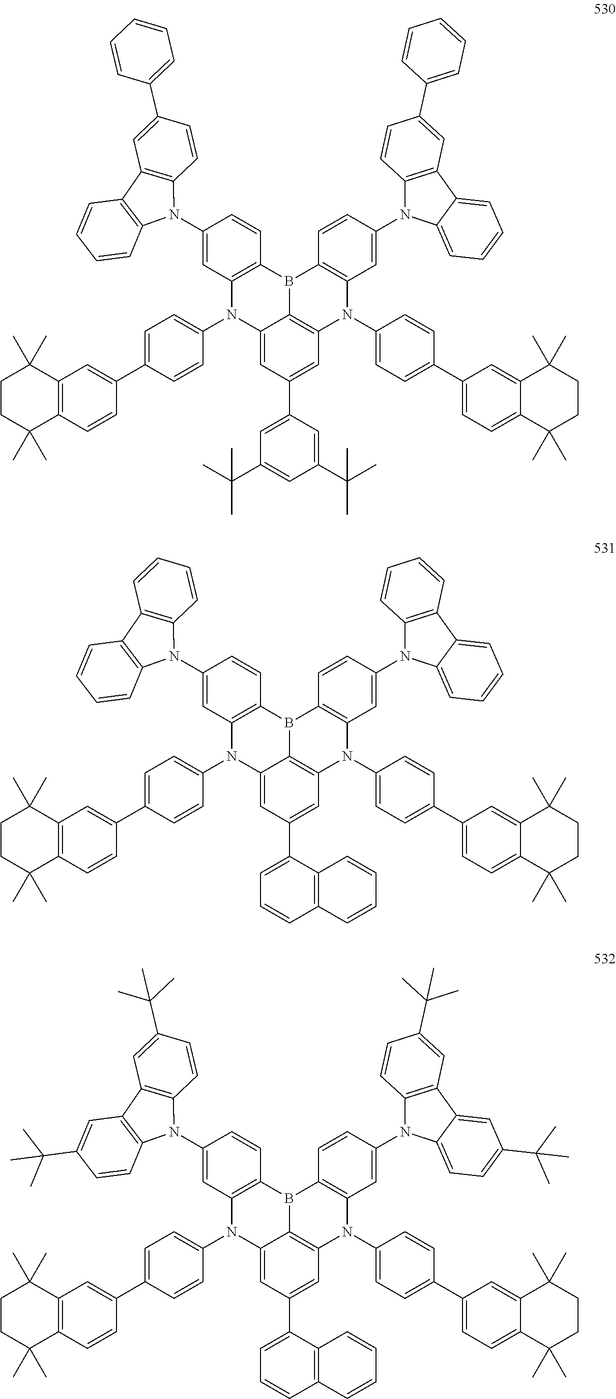

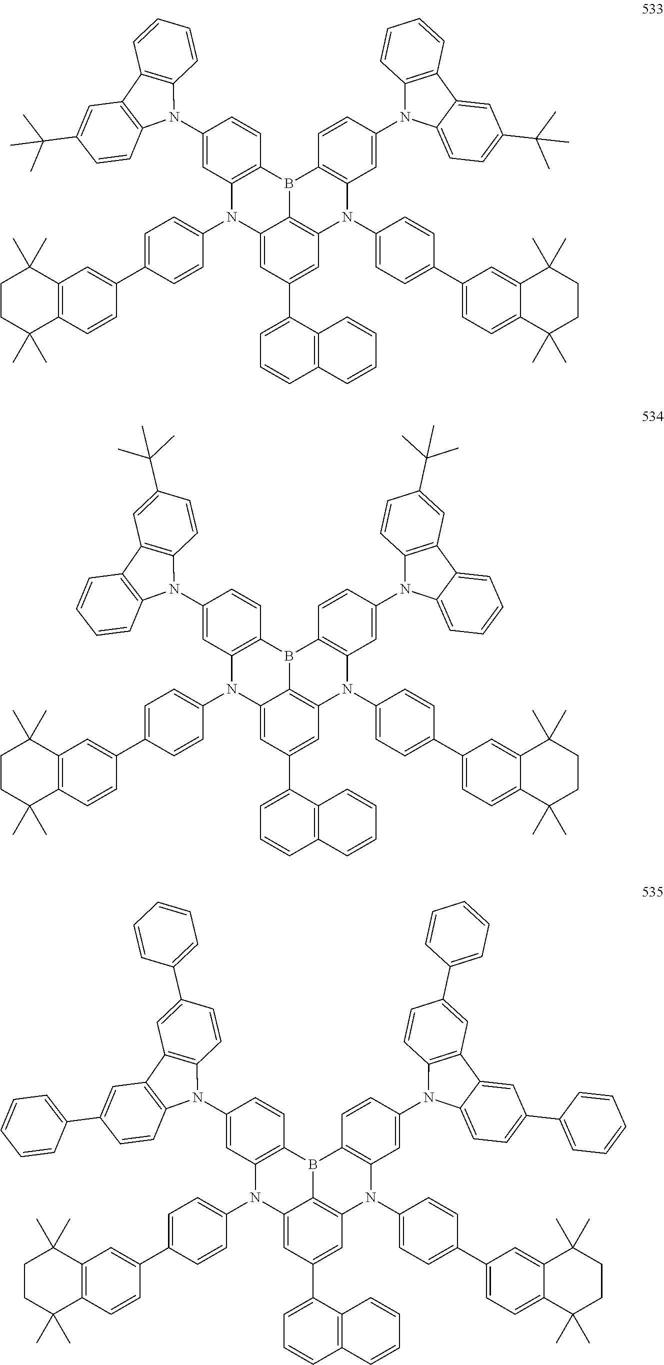

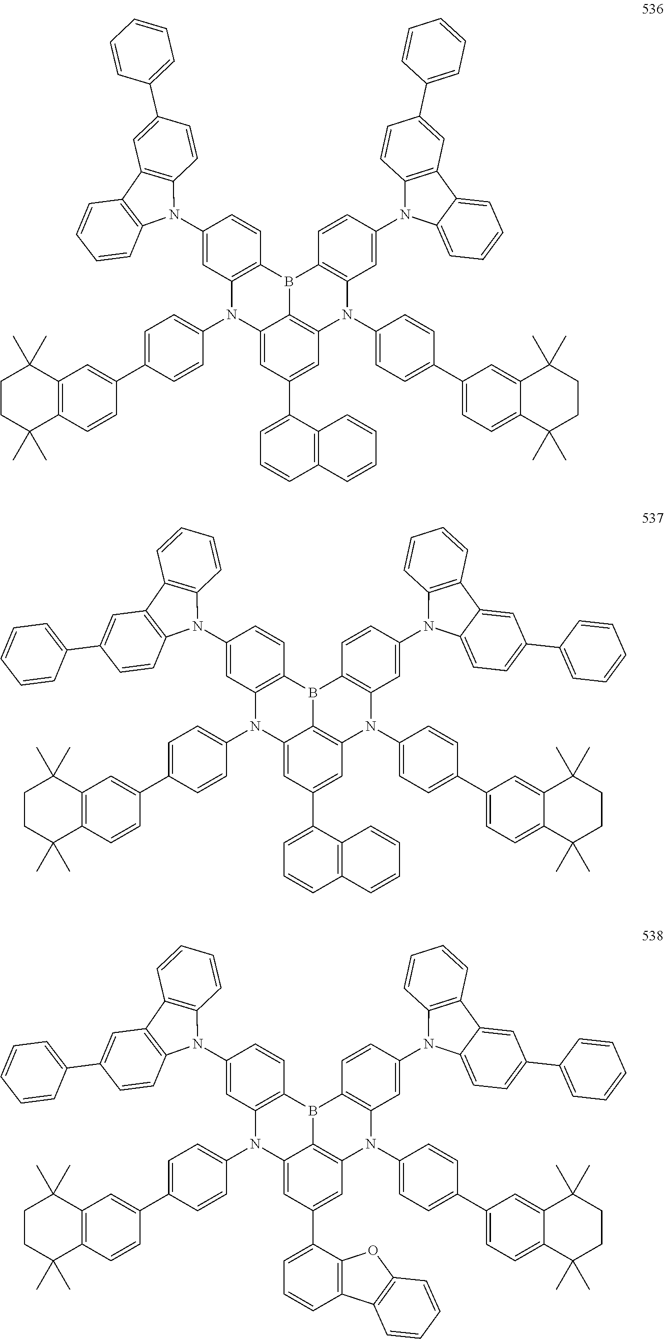

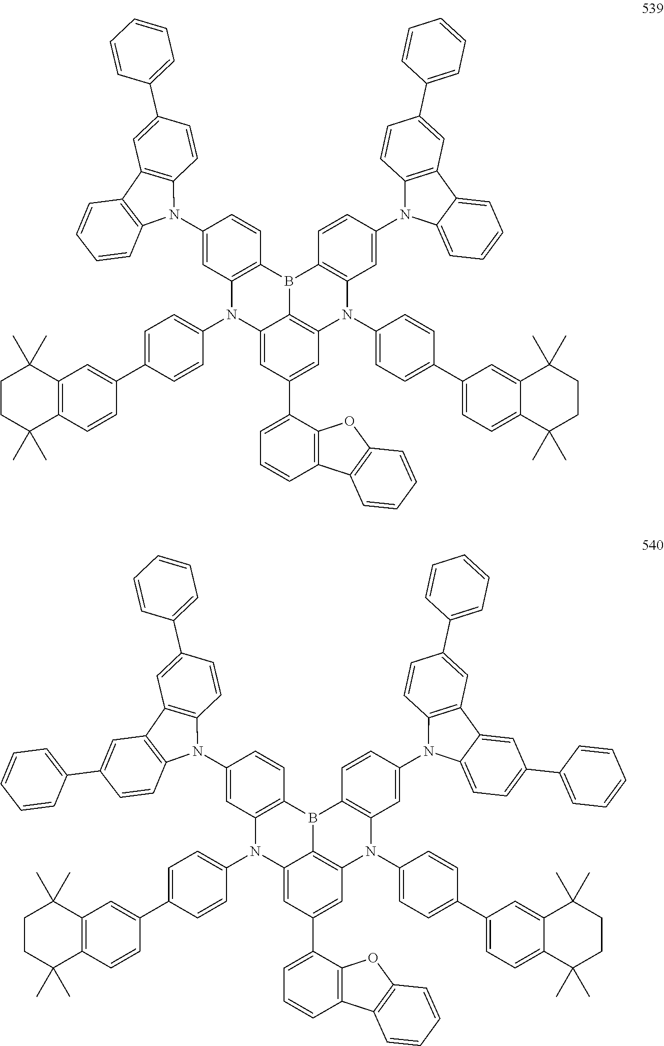 [SMILES: CC1(C)CCC(C)(C)c2cc(-c3ccc(N4c5cc(-n6c7ccccc7c7cc(-c8ccccc8)ccc76)ccc5B5c6ccc(-n7c8ccccc8c8cc(-c9ccccc9)ccc87)cc6N(c6ccc(-c7ccc8c(c7)C(C)(C)CCC8(C)C)cc6)c6cc(-c7cccc8c7oc7ccccc78)cc4c65)cc3)ccc21 ; CC1(C)CCC(C)(C)c2cc(-c3ccc(N4c5cc(-n6c7ccc(-c8ccccc8)cc7c7cc(-c8ccccc8)ccc76)ccc5B5c6ccc(-n7c8ccc(-c9ccccc9)cc8c8cc(-c9ccccc9)ccc87)cc6N(c6ccc(-c7ccc8c(c7)C(C)(C)CCC8(C)C)cc6)c6cc(-c7cccc8c7oc7ccccc78)cc4c65)cc3)ccc21]

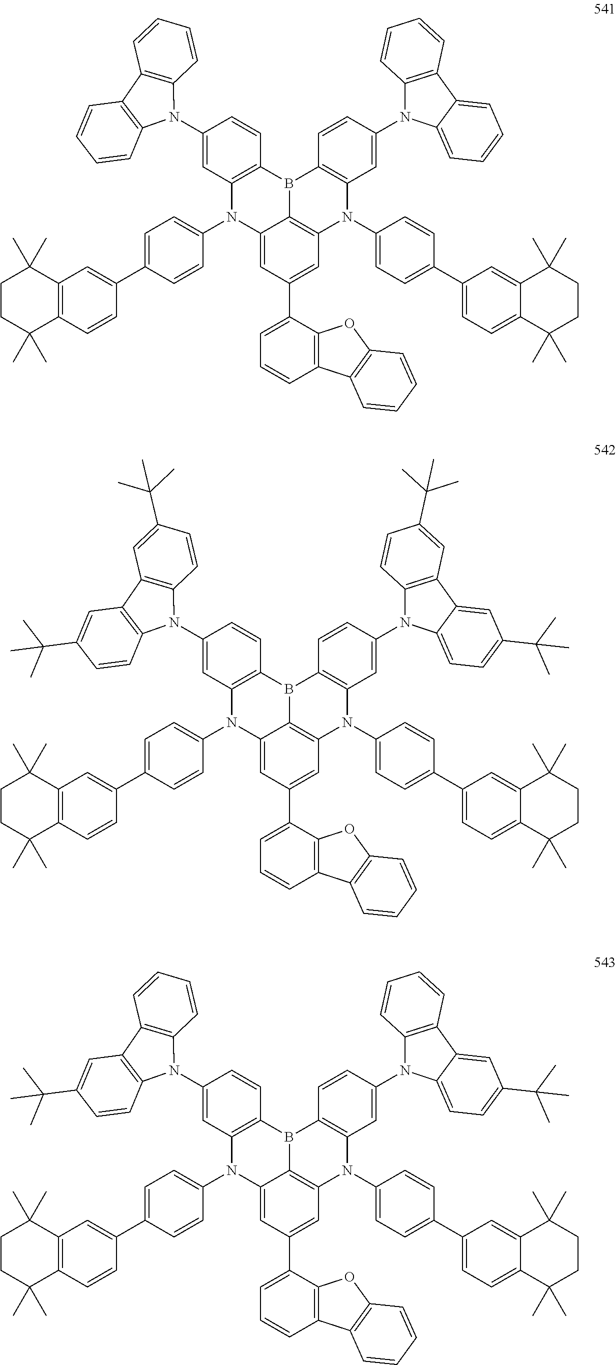

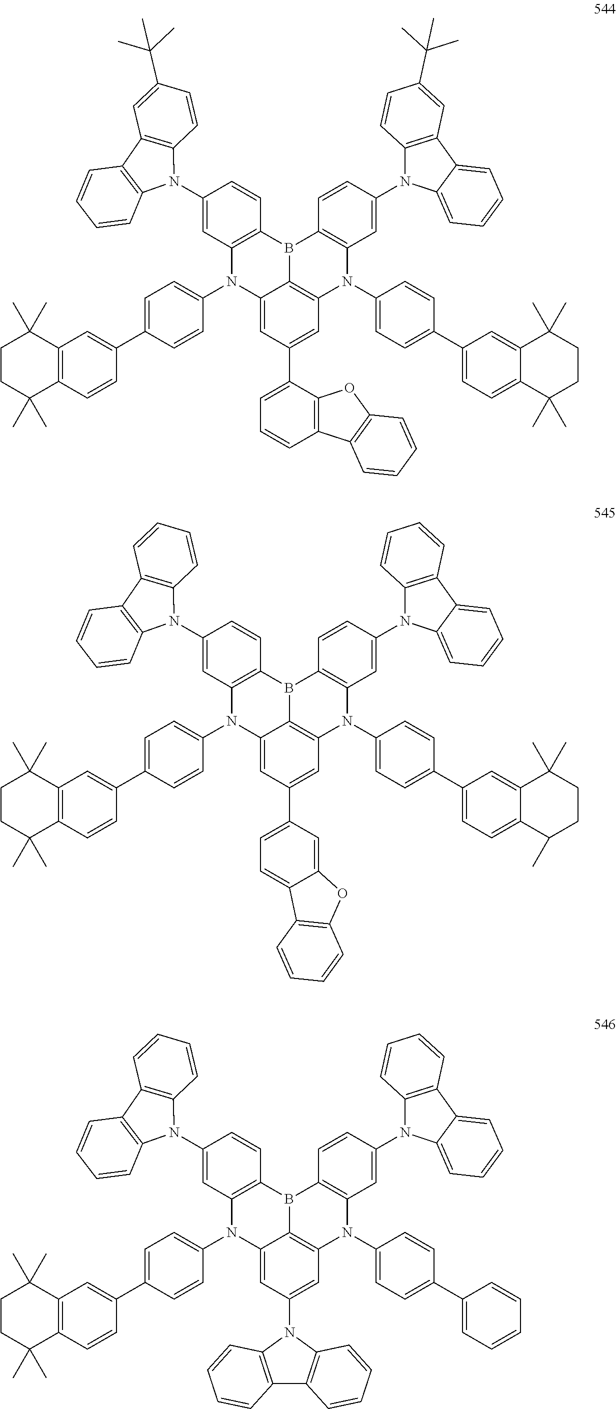

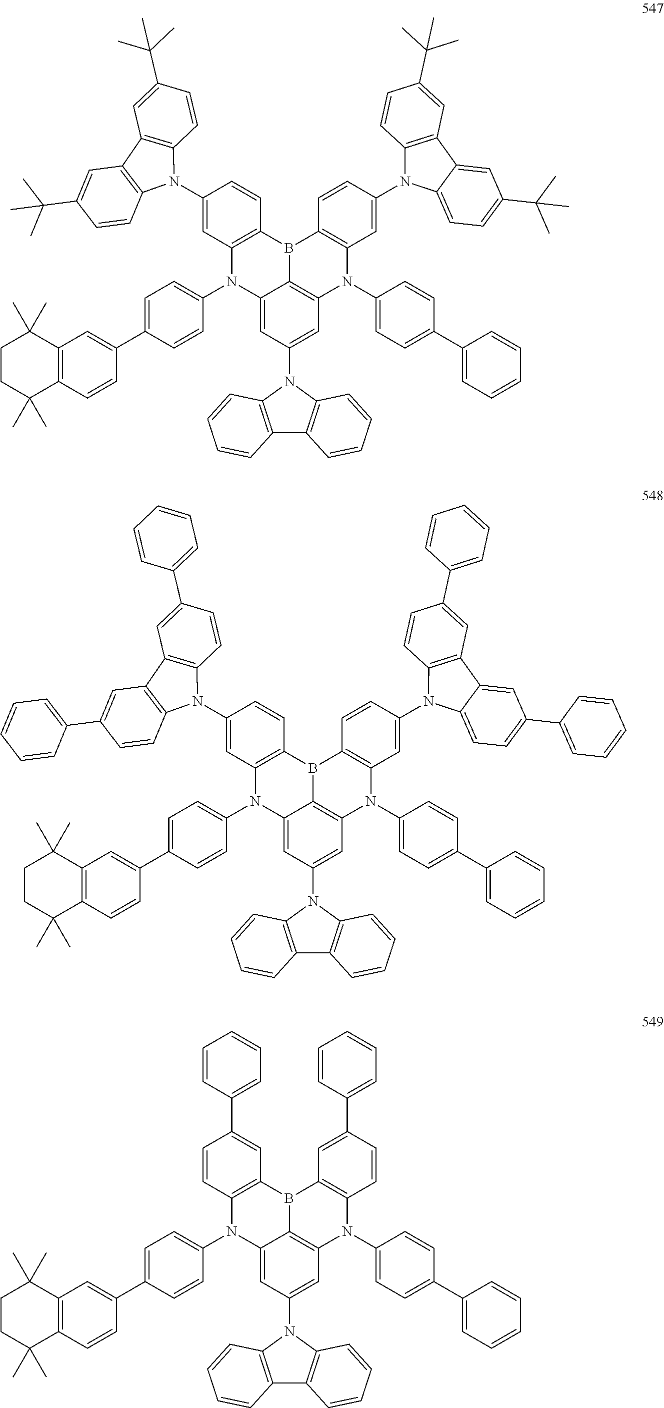

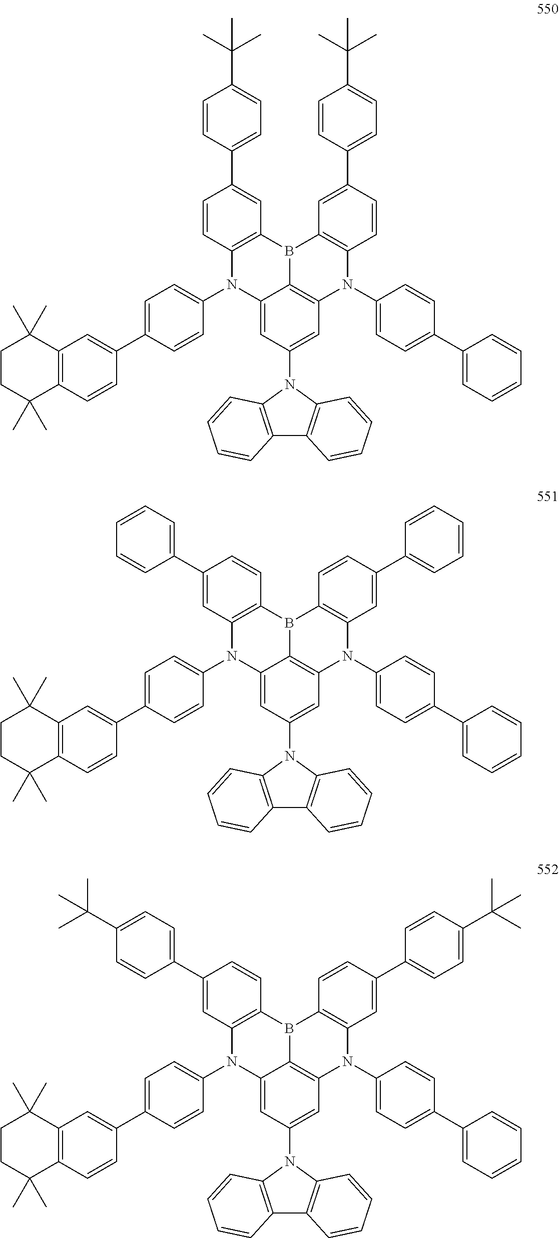 [SMILES: CC(C)(C)c1ccc(-c2ccc3c(c2)B2c4cc(-c5ccc(C(C)(C)C)cc5)ccc4N(c4ccc(-c5ccc6c(c5)C(C)(C)CCC6(C)C)cc4)c4cc(-n5c6ccccc6c6ccccc65)cc(c42)N3c2ccc(-c3ccccc3)cc2)cc1 ; CC1(C)CCC(C)(C)c2cc(-c3ccc(N4c5cc(-c6ccccc6)ccc5B5c6ccc(-c7ccccc7)cc6N(c6ccc(-c7ccccc7)cc6)c6cc(-n7c8ccccc8c8ccccc87)cc4c65)cc3)ccc21 ; CC(C)(C)c1ccc(-c2ccc3c(c2)N(c2ccc(-c4ccccc4)cc2)c2cc(-n4c5ccccc5c5ccccc54)cc4c2B3c2ccc(-c3ccc(C(C)(C)C)cc3)cc2N4c2ccc(-c3ccc4c(c3)C(C)(C)CCC4(C)C)cc2)cc1]

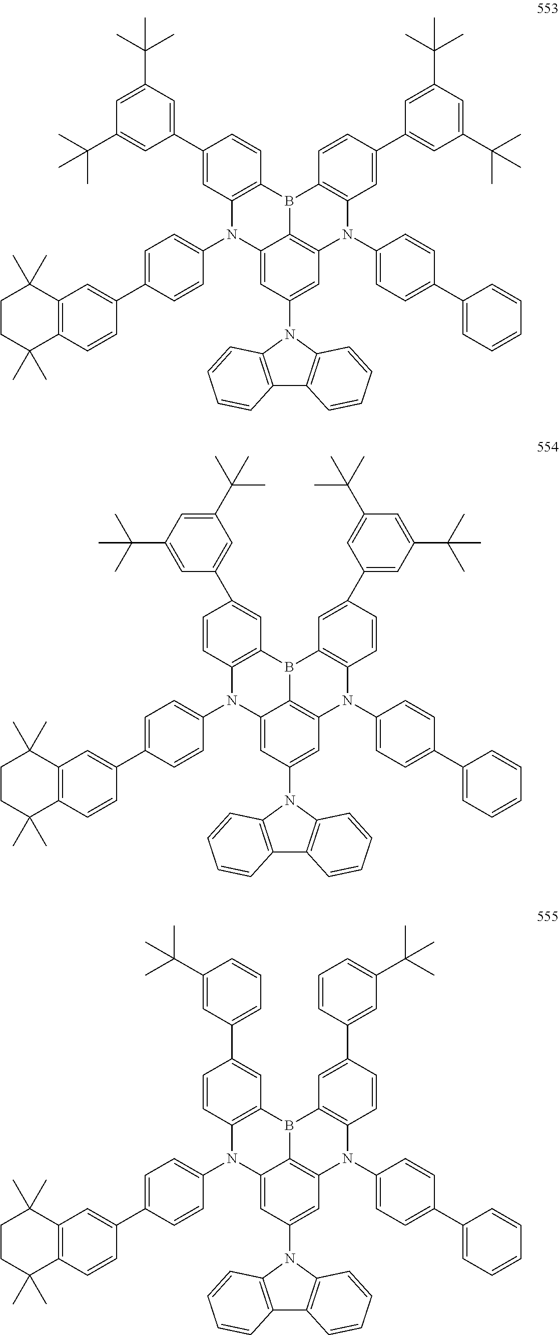

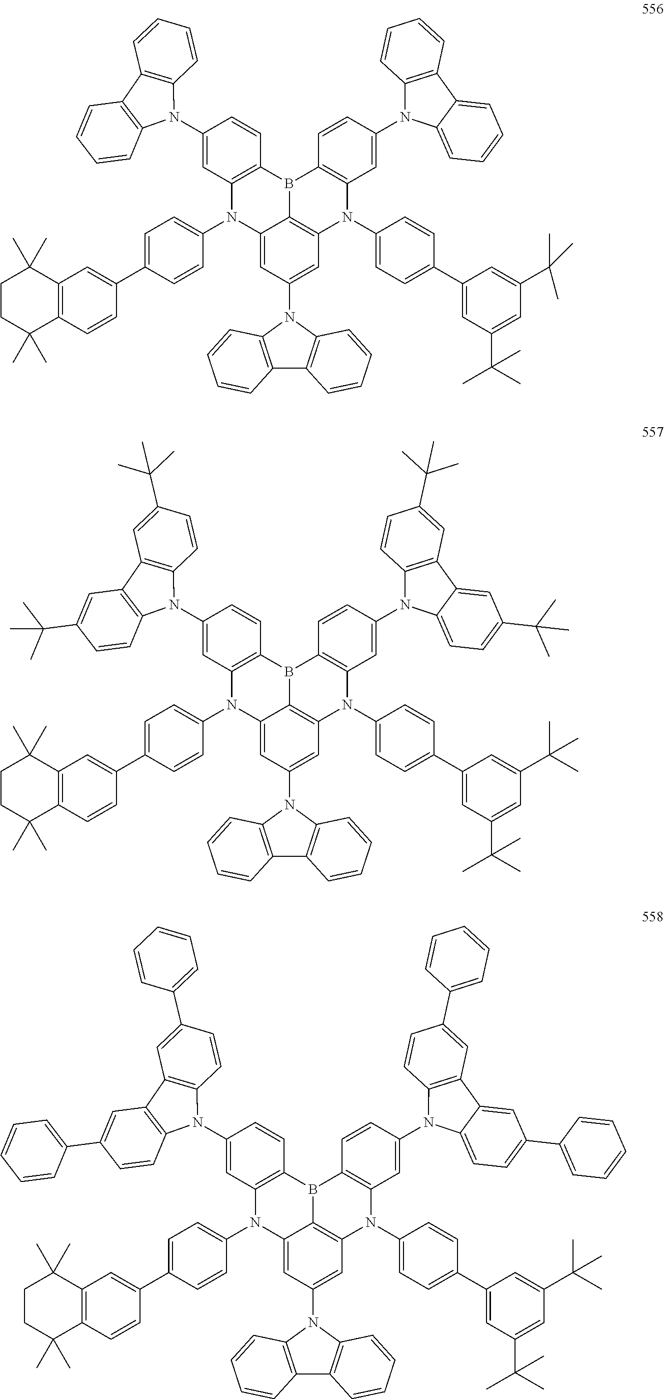

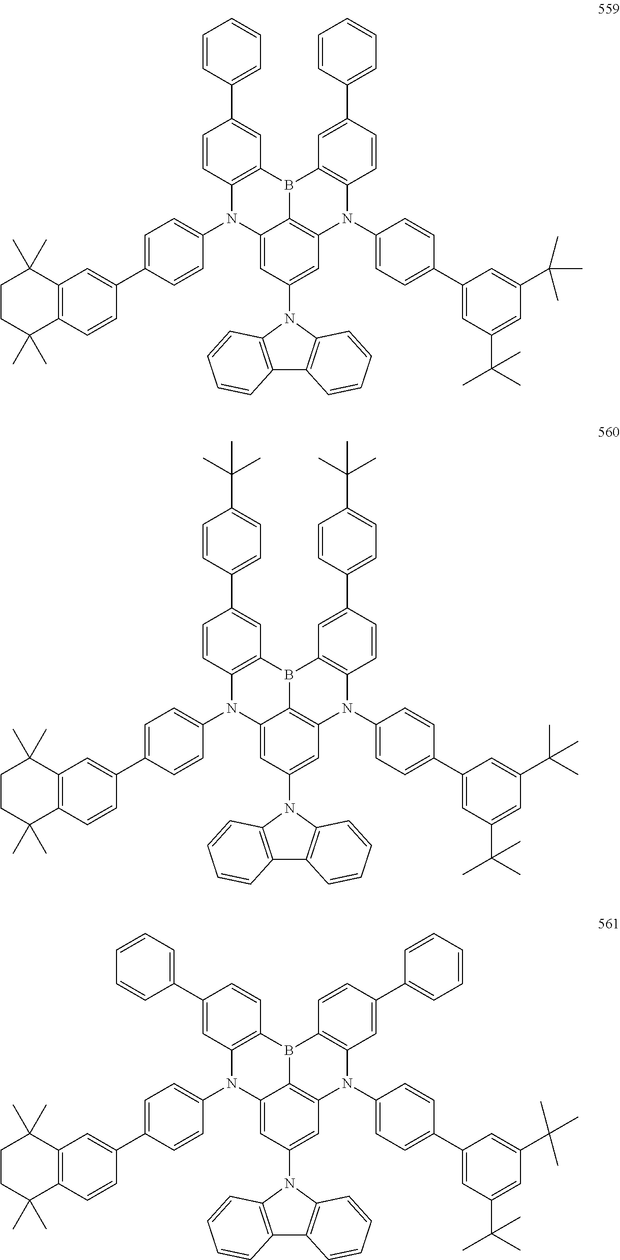 [SMILES: CC(C)(C)c1cc(-c2ccc(N3c4ccc(-c5ccccc5)cc4B4c5cc(-c6ccccc6)ccc5N(c5ccc(-c6ccc7c(c6)C(C)(C)CCC7(C)C)cc5)c5cc(-n6c7ccccc7c7ccccc76)cc3c54)cc2)cc(C(C)(C)C)c1 ; CC(C)(C)c1ccc(-c2ccc3c(c2)B2c4cc(-c5ccc(C(C)(C)C)cc5)ccc4N(c4ccc(-c5ccc6c(c5)C(C)(C)CCC6(C)C)cc4)c4cc(-n5c6ccccc6c6ccccc65)cc(c42)N3c2ccc(-c3cc(C(C)(C)C)cc(C(C)(C)C)c3)cc2)cc1 ; CC(C)(C)c1cc(-c2ccc(N3c4cc(-c5ccccc5)ccc4B4c5ccc(-c6ccccc6)cc5N(c5ccc(-c6ccc7c(c6)C(C)(C)CCC7(C)C)cc5)c5cc(-n6c7ccccc7c7ccccc76)cc3c54)cc2)cc(C(C)(C)C)c1]

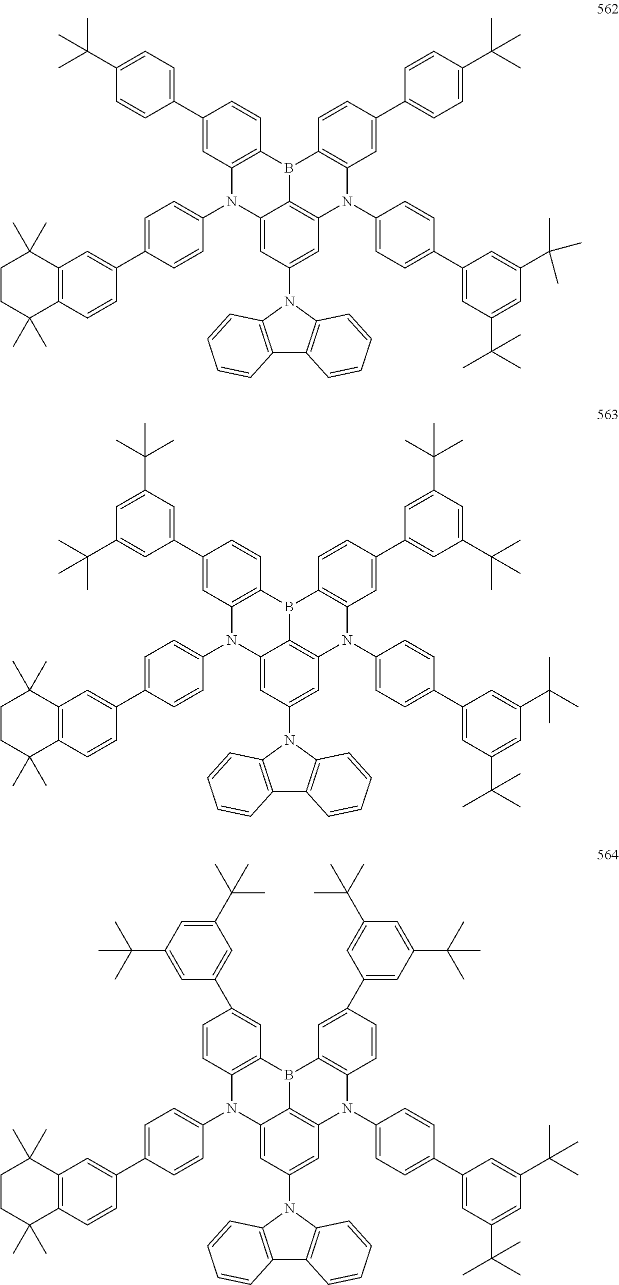

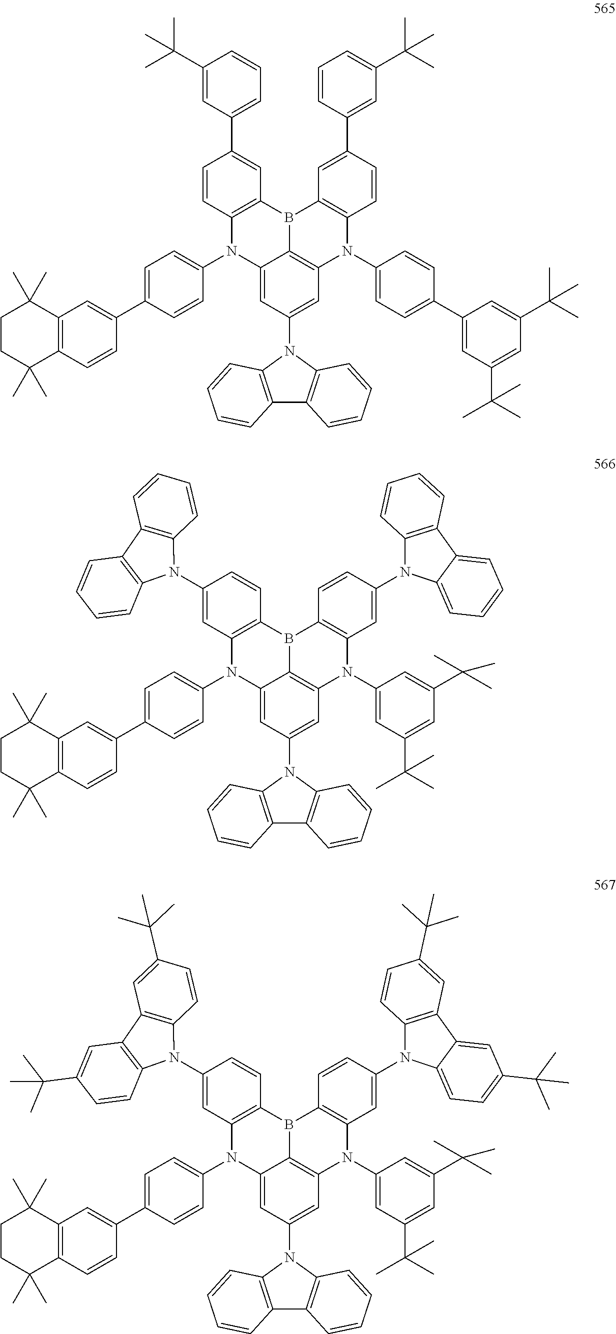

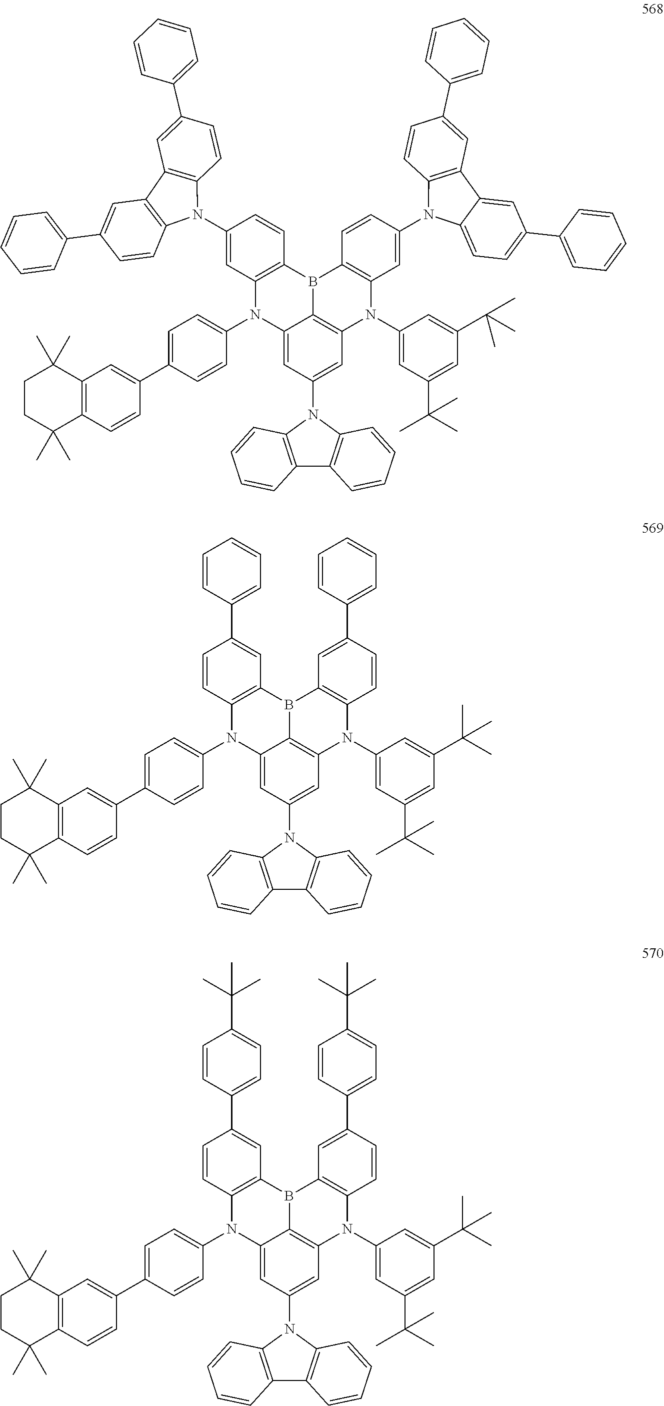

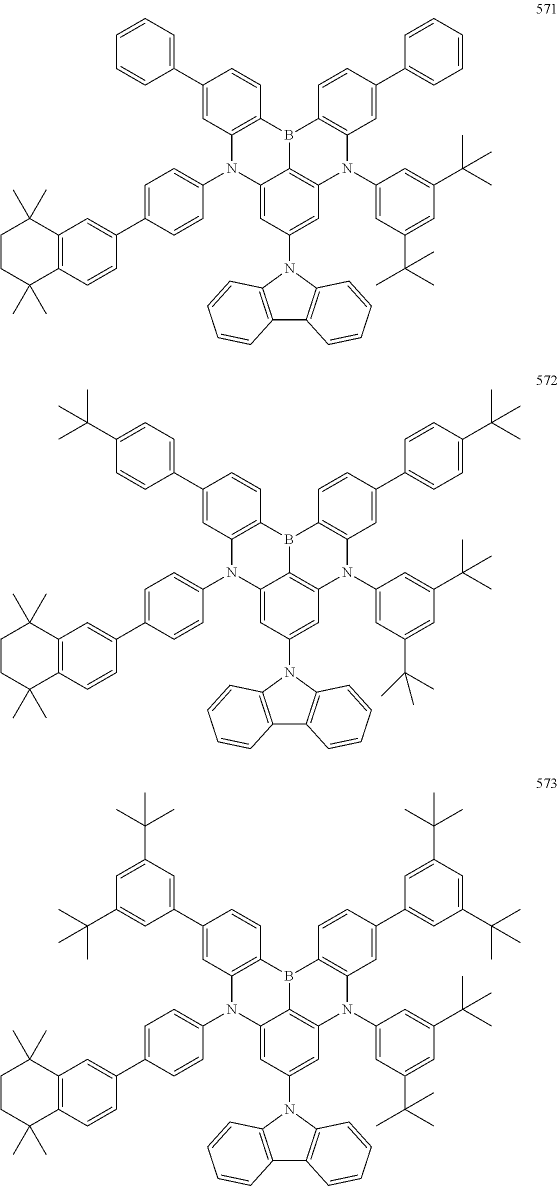 [SMILES: CC(C)(C)c1cc(N2c3cc(-c4ccccc4)ccc3B3c4ccc(-c5ccccc5)cc4N(c4ccc(-c5ccc6c(c5)C(C)(C)CCC6(C)C)cc4)c4cc(-n5c6ccccc6c6ccccc65)cc2c43)cc(C(C)(C)C)c1 ; CC(C)(C)c1ccc(-c2ccc3c(c2)N(c2ccc(-c4ccc5c(c4)C(C)(C)CCC5(C)C)cc2)c2cc(-n4c5ccccc5c5ccccc54)cc4c2B3c2ccc(-c3ccc(C(C)(C)C)cc3)cc2N4c2cc(C(C)(C)C)cc(C(C)(C)C)c2)cc1 ; CC(C)(C)c1cc(-c2ccc3c(c2)N(c2ccc(-c4ccc5c(c4)C(C)(C)CCC5(C)C)cc2)c2cc(-n4c5ccccc5c5ccccc54)cc4c2B3c2ccc(-c3cc(C(C)(C)C)cc(C(C)(C)C)c3)cc2N4c2cc(C(C)(C)C)cc(C(C)(C)C)c2)cc(C(C)(C)C)c1]

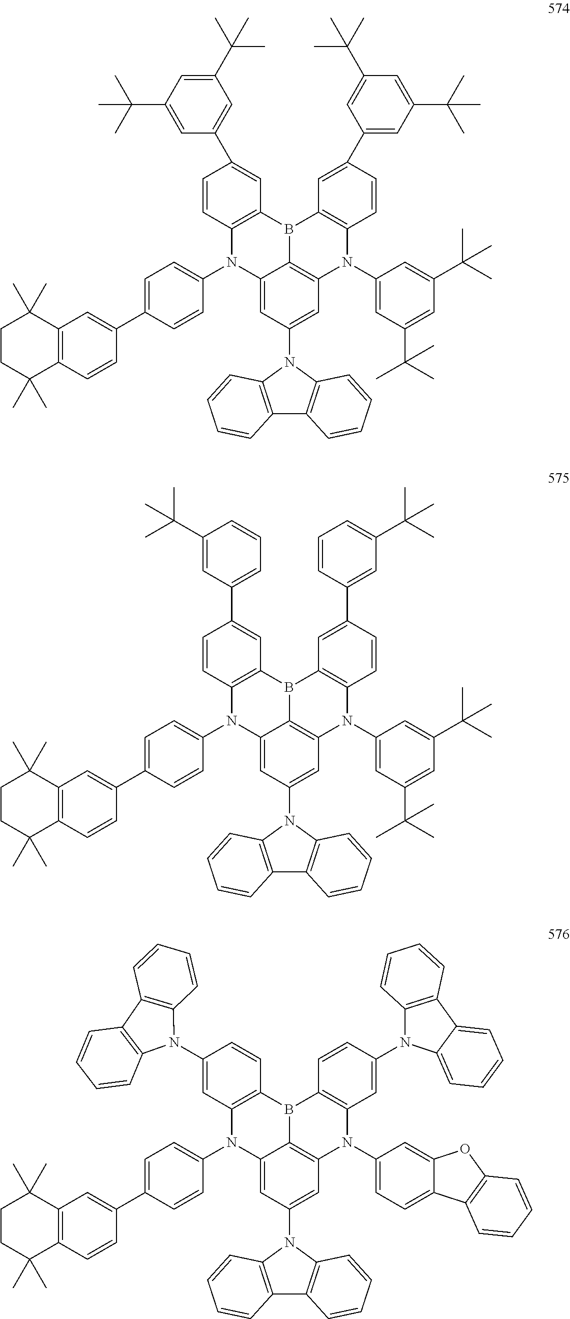

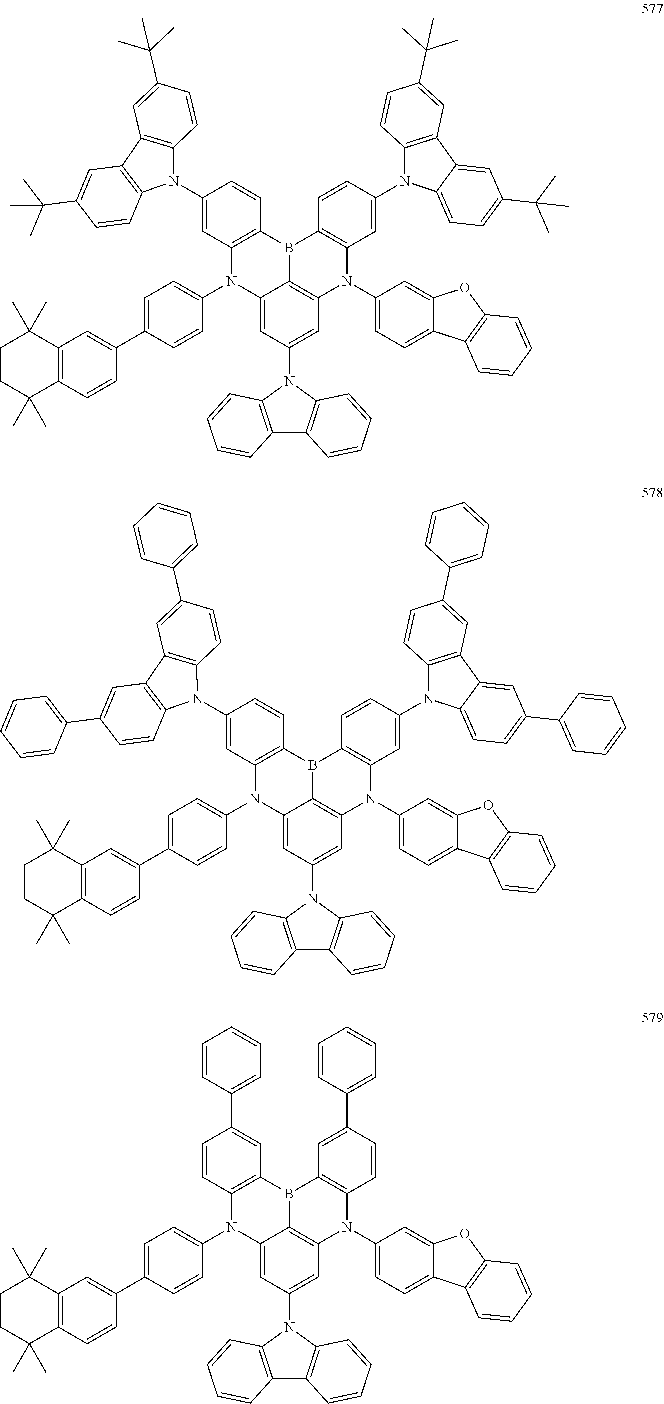

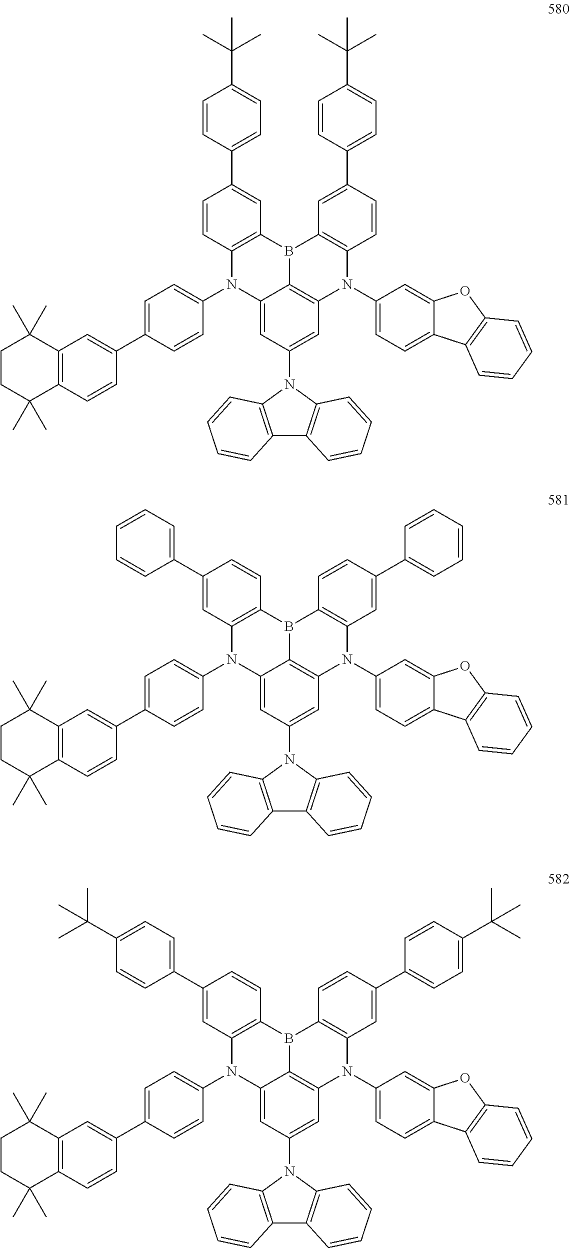 [SMILES: CC(C)(C)c1ccc(-c2ccc3c(c2)B2c4cc(-c5ccc(C(C)(C)C)cc5)ccc4N(c4ccc5c(c4)oc4ccccc45)c4cc(-n5c6ccccc6c6ccccc65)cc(c42)N3c2ccc(-c3ccc4c(c3)C(C)(C)CCC4(C)C)cc2)cc1 ; CC1(C)CCC(C)(C)c2cc(-c3ccc(N4c5cc(-c6ccccc6)ccc5B5c6ccc(-c7ccccc7)cc6N(c6ccc7c(c6)oc6ccccc67)c6cc(-n7c8ccccc8c8ccccc87)cc4c65)cc3)ccc21 ; CC(C)(C)c1ccc(-c2ccc3c(c2)N(c2ccc(-c4ccc5c(c4)C(C)(C)CCC5(C)C)cc2)c2cc(-n4c5ccccc5c5ccccc54)cc4c2B3c2ccc(-c3ccc(C(C)(C)C)cc3)cc2N4c2ccc3c(c2)oc2ccccc23)cc1]

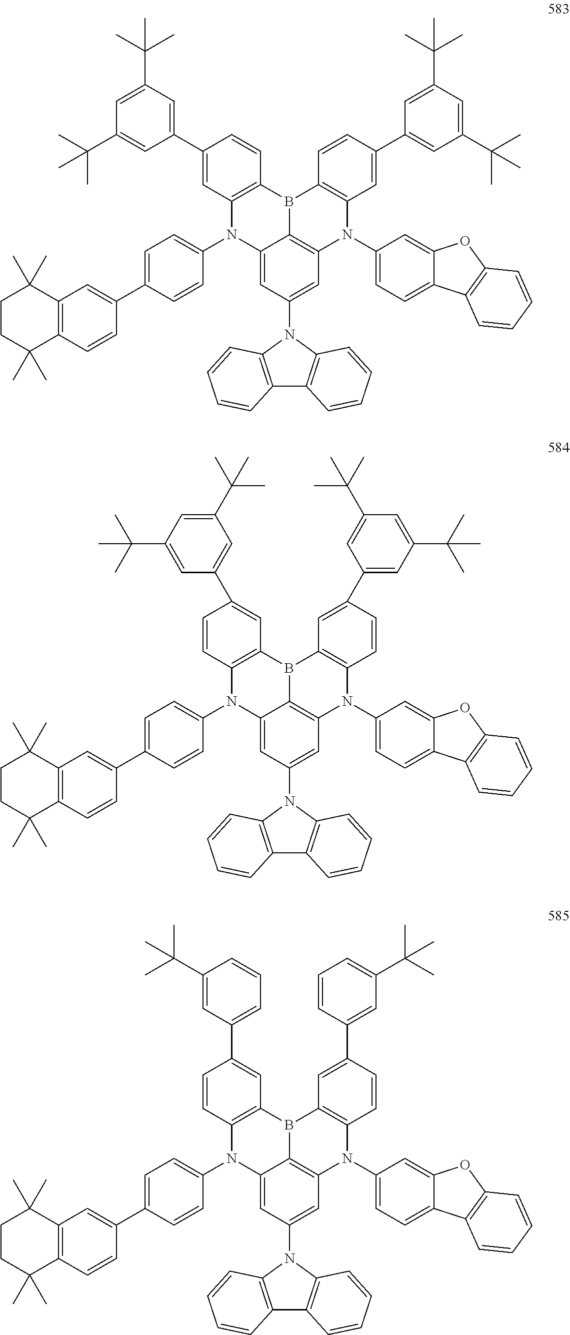

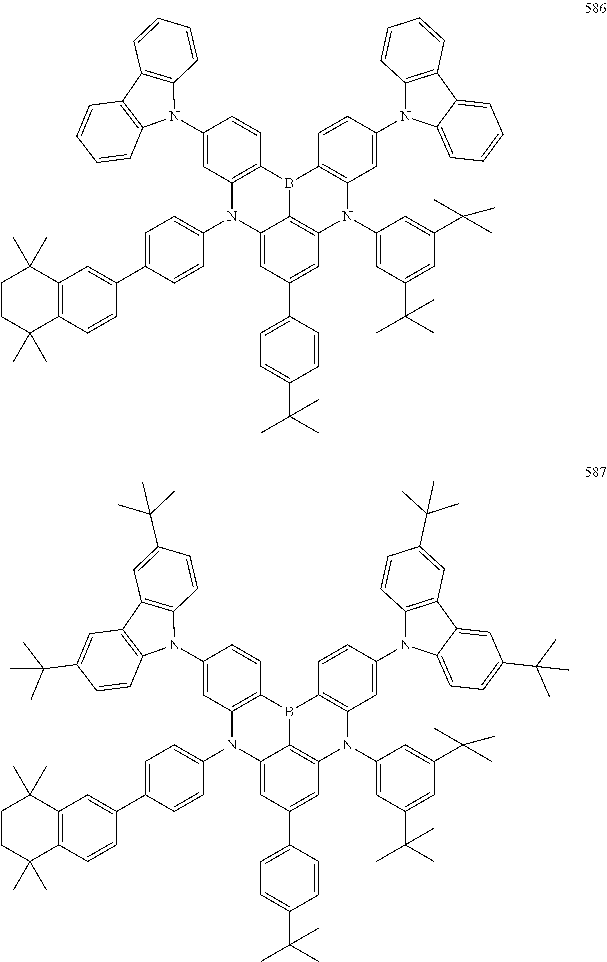 [SMILES: CC(C)(C)c1ccc(-c2cc3c4c(c2)N(c2cc(C(C)(C)C)cc(C(C)(C)C)c2)c2cc(-n5c6ccccc6c6ccccc65)ccc2B4c2ccc(-n4c5ccccc5c5ccccc54)cc2N3c2ccc(-c3ccc4c(c3)C(C)(C)CCC4(C)C)cc2)cc1 ; CC(C)(C)c1ccc(-c2cc3c4c(c2)N(c2cc(C(C)(C)C)cc(C(C)(C)C)c2)c2cc(-n5c6ccc(C(C)(C)C)cc6c6cc(C(C)(C)C)ccc65)ccc2B4c2ccc(-n4c5ccc(C(C)(C)C)cc5c5cc(C(C)(C)C)ccc54)cc2N3c2ccc(-c3ccc4c(c3)C(C)(C)CCC4(C)C)cc2)cc1]

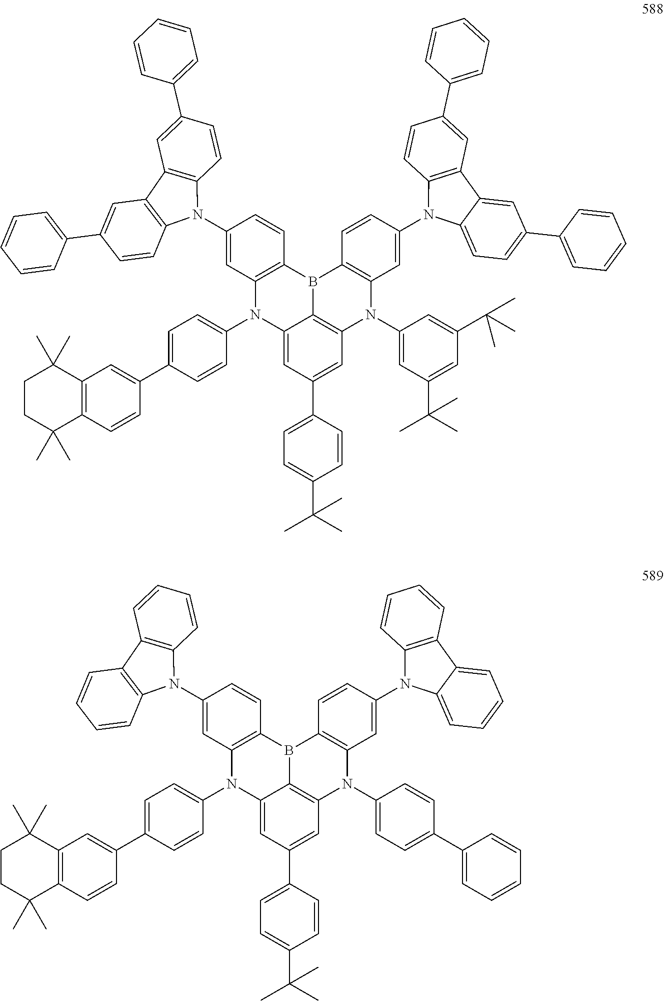 [SMILES: CC(C)(C)c1ccc(-c2cc3c4c(c2)N(c2cc(C(C)(C)C)cc(C(C)(C)C)c2)c2cc(-n5c6ccc(-c7ccccc7)cc6c6cc(-c7ccccc7)ccc65)ccc2B4c2ccc(-n4c5ccc(-c6ccccc6)cc5c5cc(-c6ccccc6)ccc54)cc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccccc6c6ccccc65)ccc2B4c2ccc(-n4c5ccccc5c5ccccc54)cc2N3c2ccc(-c3ccccc3)cc2)cc1]

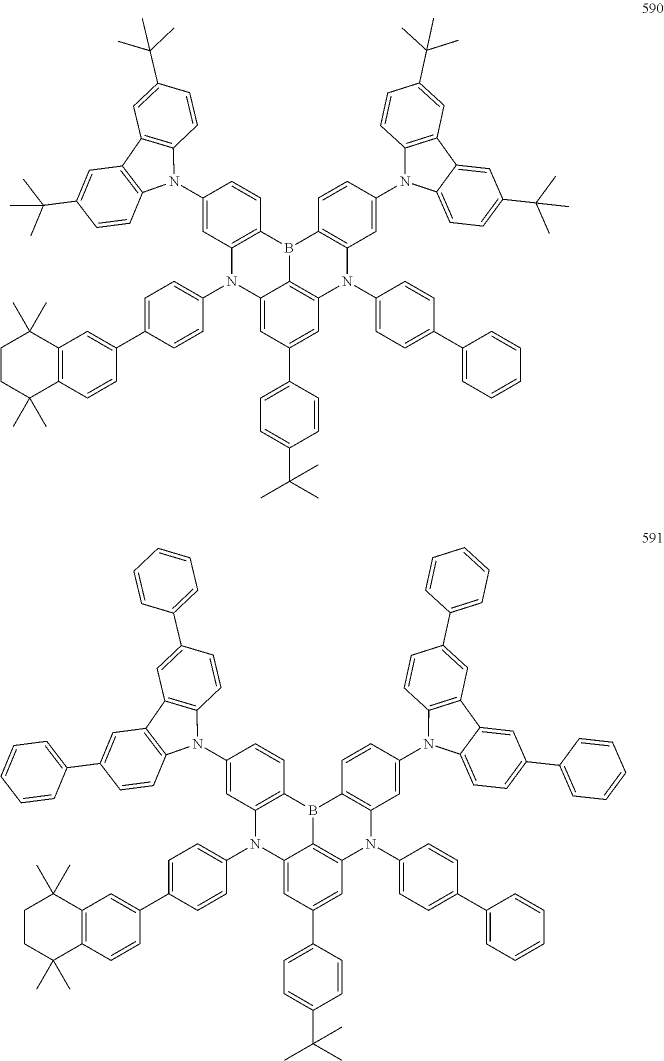 [SMILES: CC(C)(C)c1ccc(-c2cc3c4c(c2)N(c2ccc(-c5ccc6c(c5)C(C)(C)CCC6(C)C)cc2)c2cc(-n5c6ccc(C(C)(C)C)cc6c6cc(C(C)(C)C)ccc65)ccc2B4c2ccc(-n4c5ccc(C(C)(C)C)cc5c5cc(C(C)(C)C)ccc54)cc2N3c2ccc(-c3ccccc3)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2ccc(-n4c5ccc(-c6ccccc6)cc5c5cc(-c6ccccc6)ccc54)cc2N3c2ccc(-c3ccccc3)cc2)cc1]

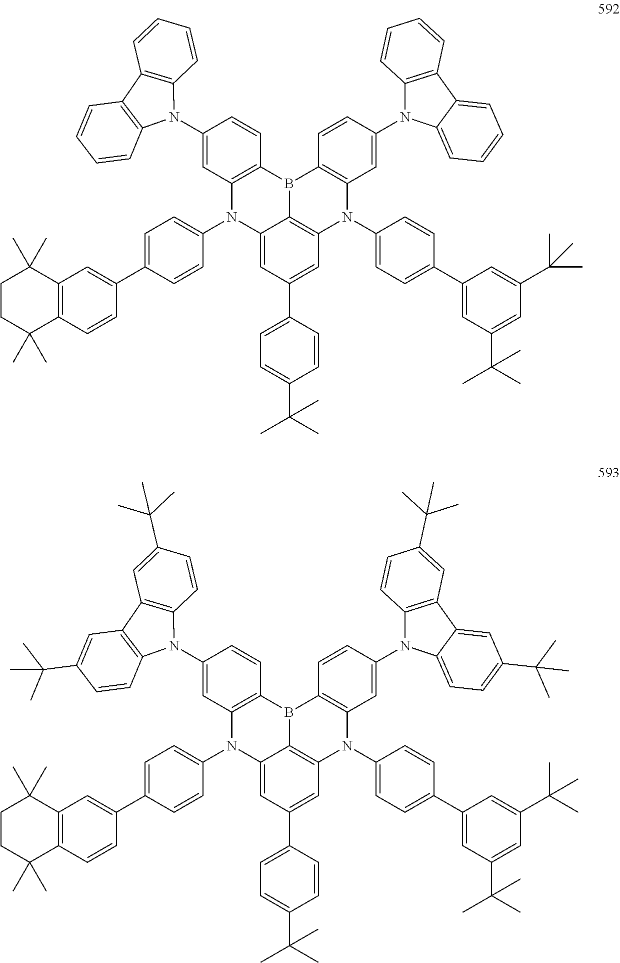 [SMILES: CC(C)(C)c1ccc(-c2cc3c4c(c2)N(c2ccc(-c5ccc6c(c5)C(C)(C)CCC6(C)C)cc2)c2cc(-n5c6ccccc6c6ccccc65)ccc2B4c2ccc(-n4c5ccccc5c5ccccc54)cc2N3c2ccc(-c3cc(C(C)(C)C)cc(C(C)(C)C)c3)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccc(C(C)(C)C)cc6c6cc(C(C)(C)C)ccc65)ccc2B4c2ccc(-n4c5ccc(C(C)(C)C)cc5c5cc(C(C)(C)C)ccc54)cc2N3c2ccc(-c3cc(C(C)(C)C)cc(C(C)(C)C)c3)cc2)cc1]

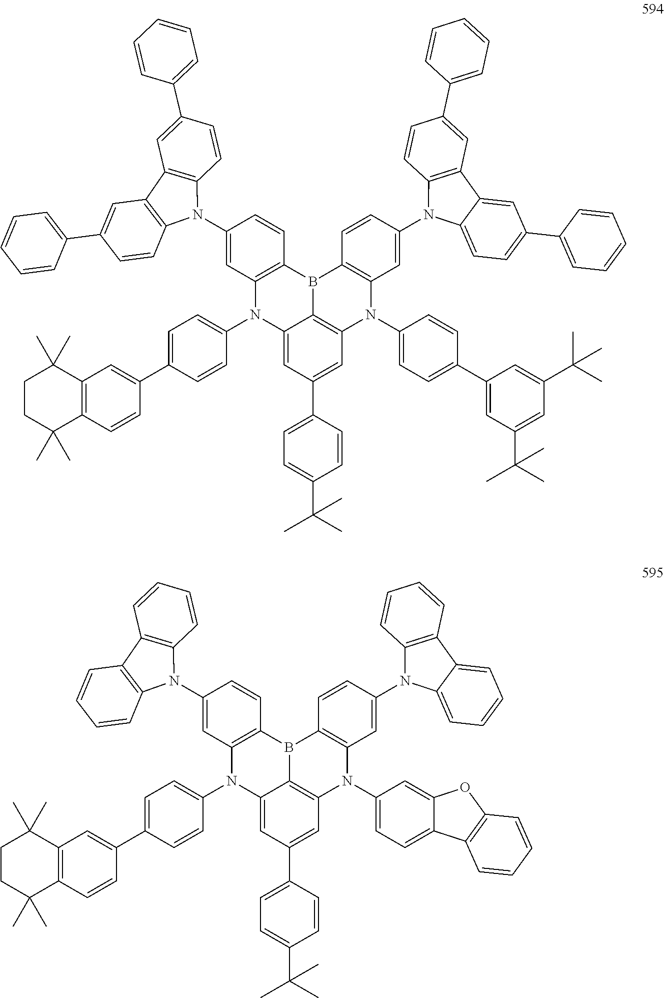 [SMILES: CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2ccc(-n4c5ccc(-c6ccccc6)cc5c5cc(-c6ccccc6)ccc54)cc2N3c2ccc(-c3cc(C(C)(C)C)cc(C(C)(C)C)c3)cc2)cc1 ; CC(C)(C)c1ccc(-c2cc3c4c(c2)N(c2ccc5c(c2)oc2ccccc25)c2cc(-n5c6ccccc6c6ccccc65)ccc2B4c2ccc(-n4c5ccccc5c5ccccc54)cc2N3c2ccc(-c3ccc4c(c3)C(C)(C)CCC4(C)C)cc2)cc1]

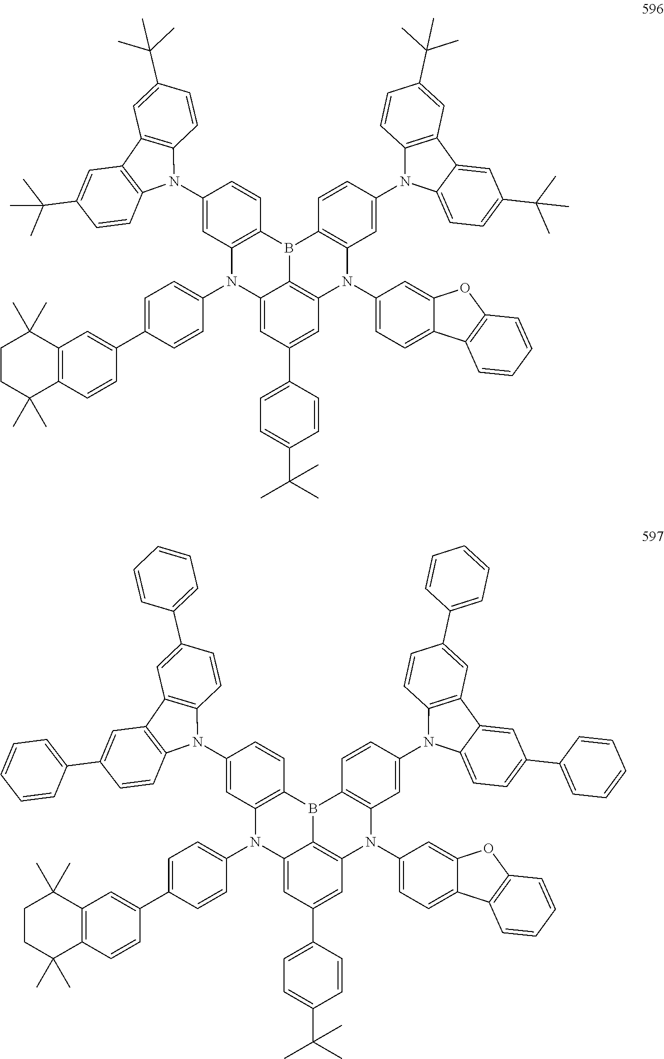 [SMILES: CC(C)(C)c1ccc(-c2cc3c4c(c2)N(c2ccc5c(c2)oc2ccccc25)c2cc(-n5c6ccc(C(C)(C)C)cc6c6cc(C(C)(C)C)ccc65)ccc2B4c2ccc(-n4c5ccc(C(C)(C)C)cc5c5cc(C(C)(C)C)ccc54)cc2N3c2ccc(-c3ccc4c(c3)C(C)(C)CCC4(C)C)cc2)cc1 ; CC(C)(C)c1ccc(-c2cc3c4c(c2)N(c2ccc5c(c2)oc2ccccc25)c2cc(-n5c6ccc(-c7ccccc7)cc6c6cc(-c7ccccc7)ccc65)ccc2B4c2ccc(-n4c5ccc(-c6ccccc6)cc5c5cc(-c6ccccc6)ccc54)cc2N3c2ccc(-c3ccc4c(c3)C(C)(C)CCC4(C)C)cc2)cc1]

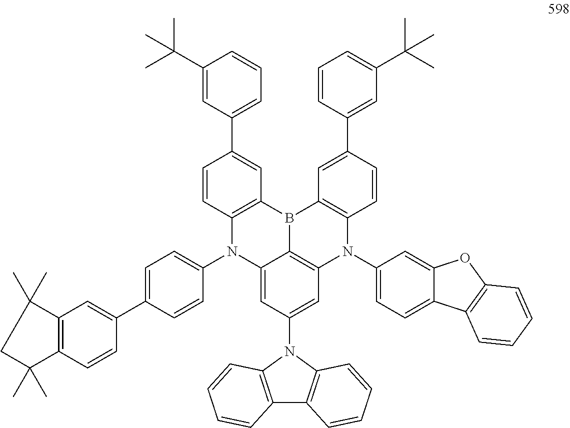 [SMILES: CC(C)(C)c1cccc(-c2ccc3c(c2)B2c4cc(-c5cccc(C(C)(C)C)c5)ccc4N(c4ccc5c(c4)oc4ccccc45)c4cc(-n5c6ccccc6c6ccccc65)cc(c42)N3c2ccc(-c3ccc4c(c3)C(C)(C)CC4(C)C)cc2)c1]

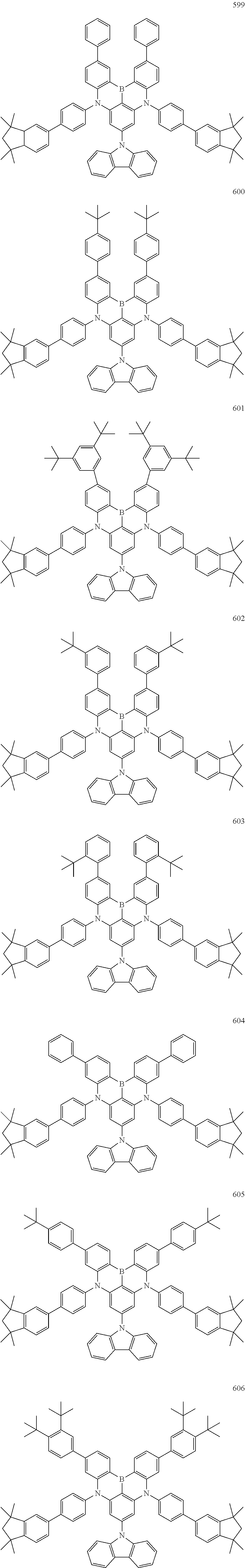

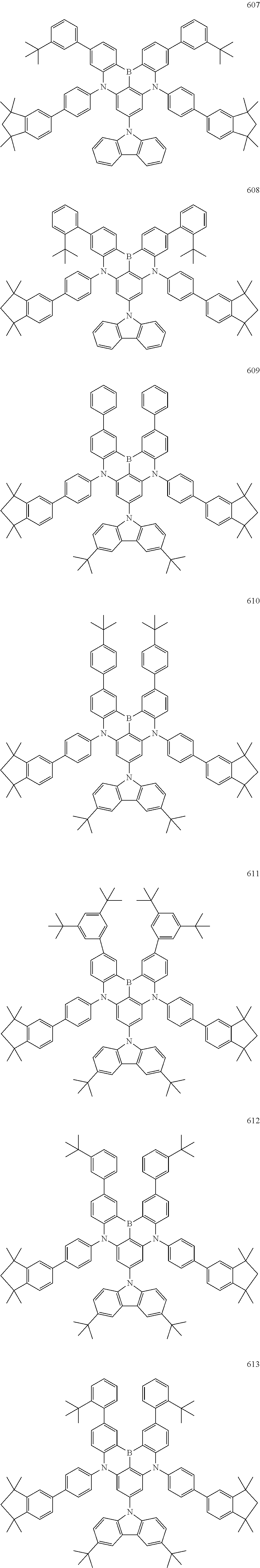

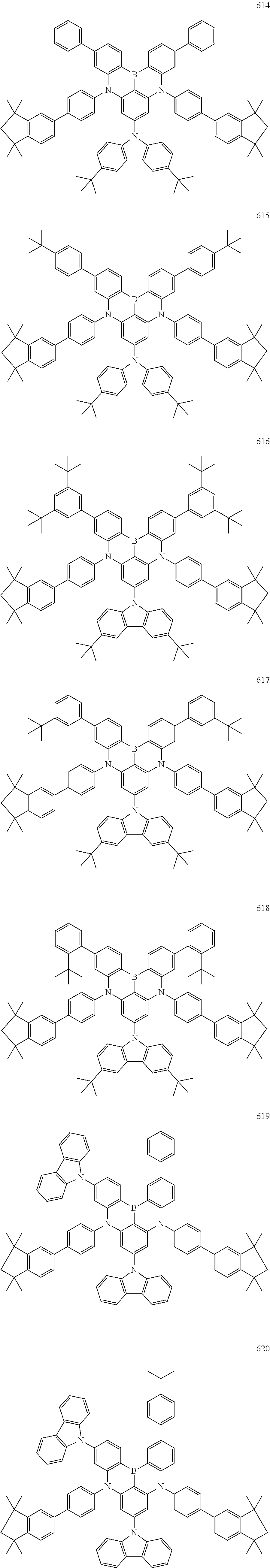

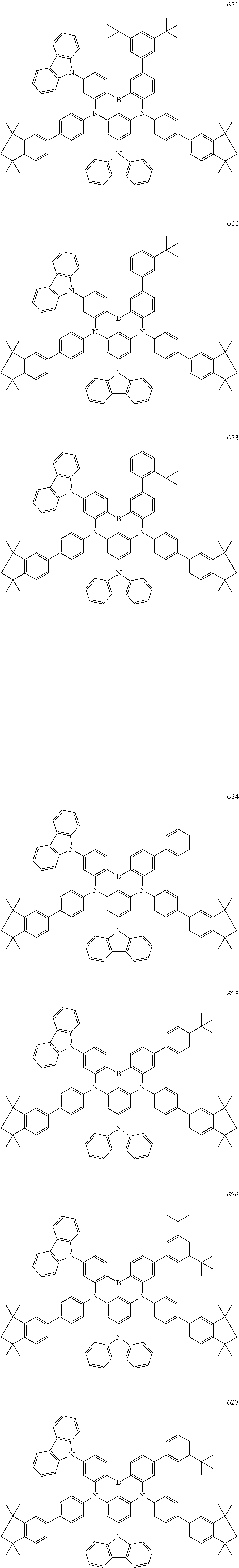

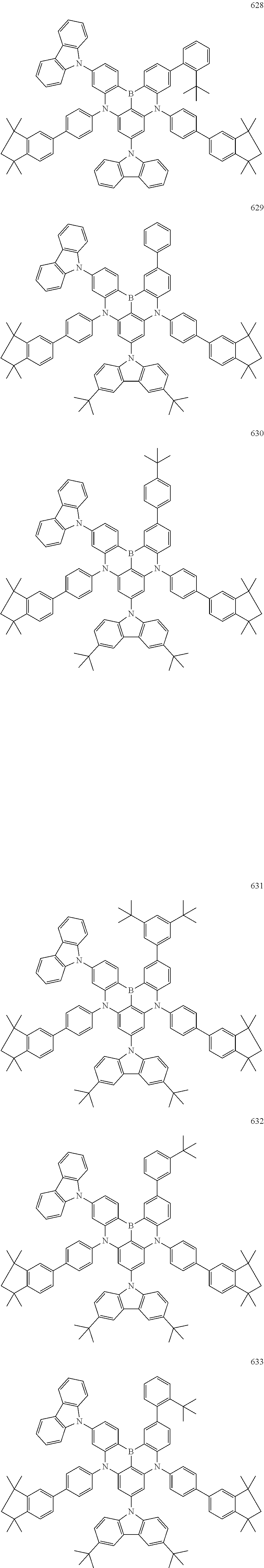

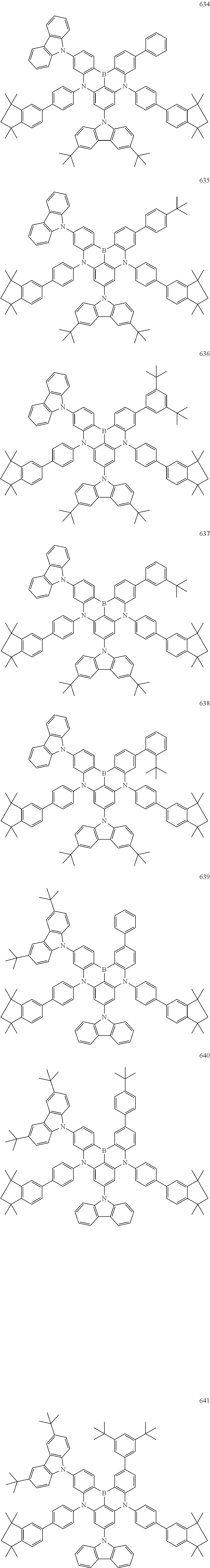

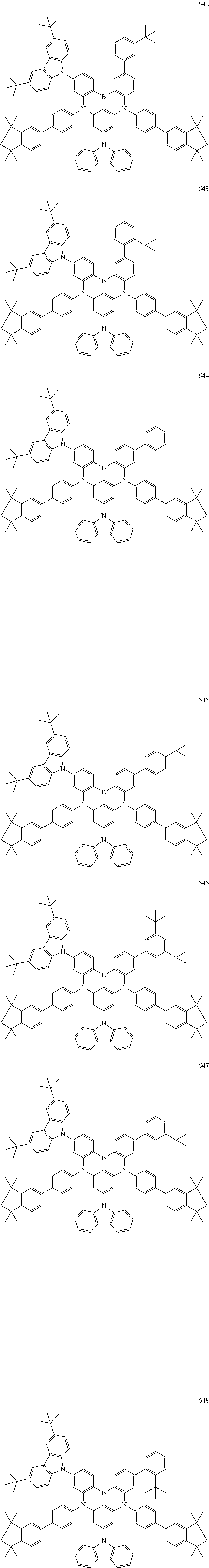

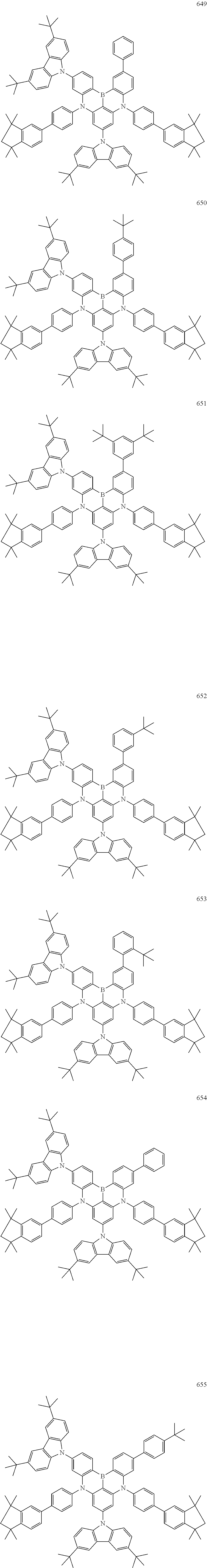

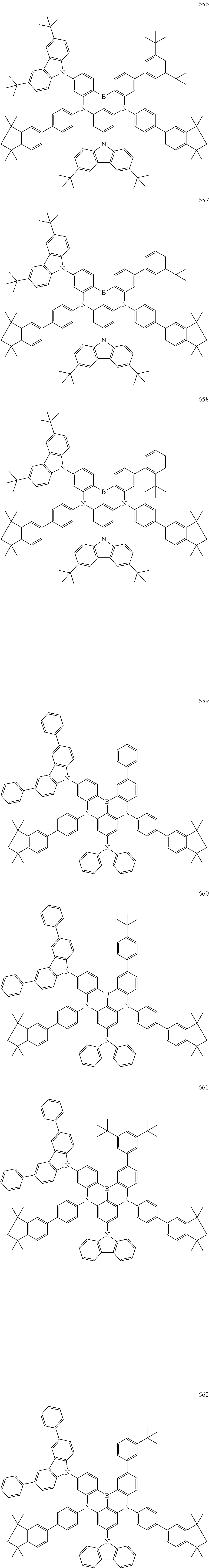

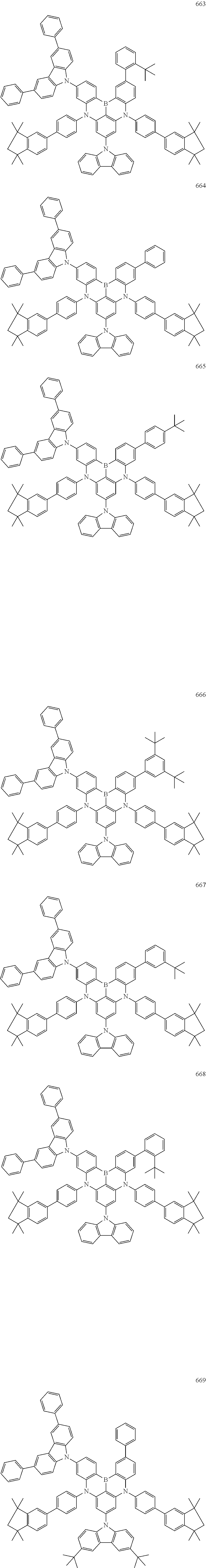

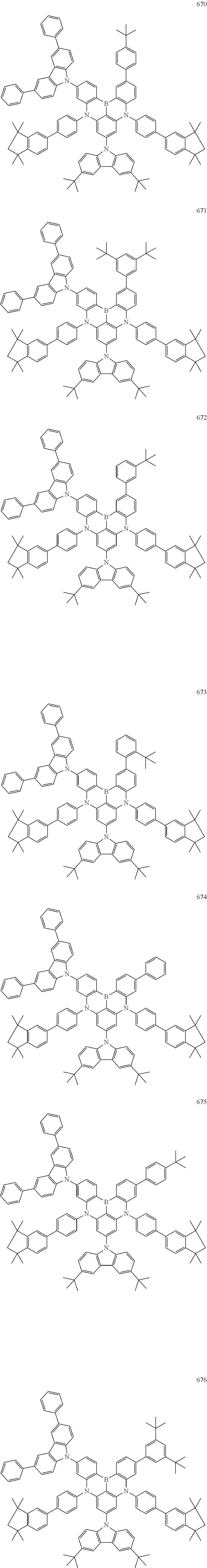

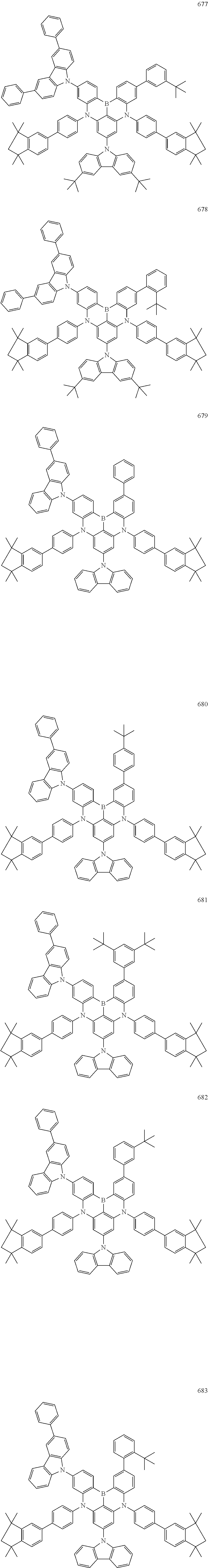

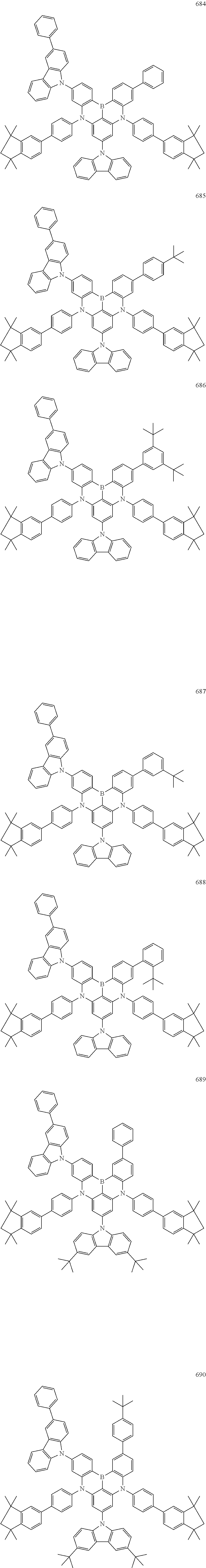

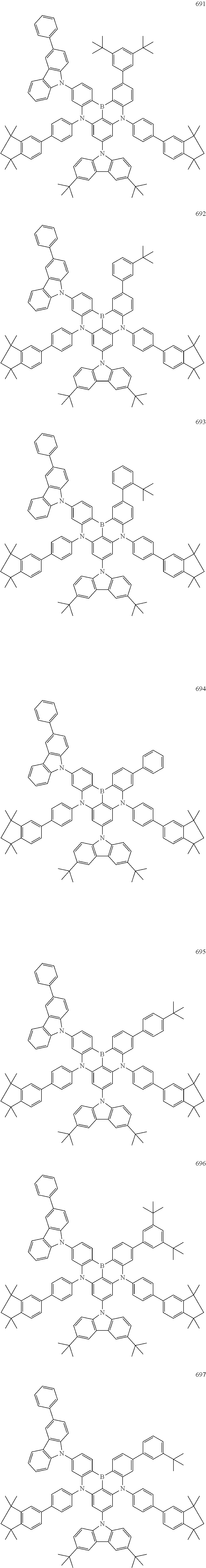

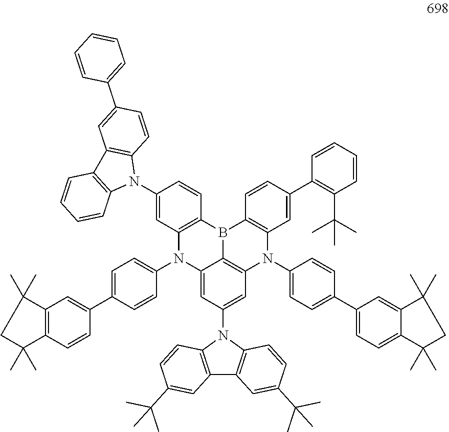 [SMILES: CC(C)(C)c1ccc2c(c1)c1cc(C(C)(C)C)ccc1n2-c1cc2c3c(c1)N(c1ccc(-c4ccc5c(c4)C(C)(C)CC5(C)C)cc1)c1cc(-n4c5ccccc5c5cc(-c6ccccc6)ccc54)ccc1B3c1ccc(-c3ccccc3C(C)(C)C)cc1N2c1ccc(-c2ccc3c(c2)C(C)(C)CC3(C)C)cc1]

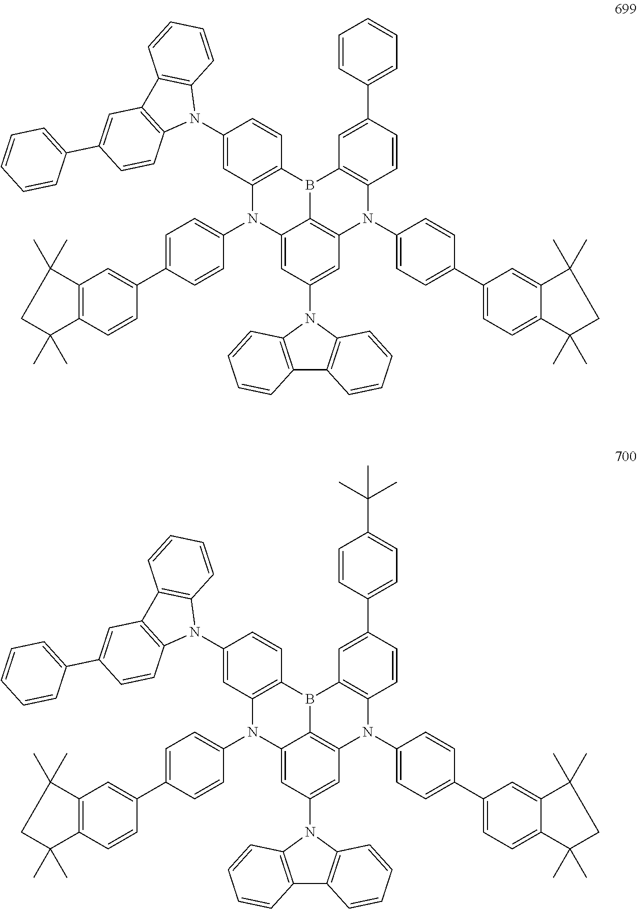 [SMILES: CC1(C)CC(C)(C)c2cc(-c3ccc(N4c5ccc(-c6ccccc6)cc5B5c6ccc(-n7c8ccccc8c8cc(-c9ccccc9)ccc87)cc6N(c6ccc(-c7ccc8c(c7)C(C)(C)CC8(C)C)cc6)c6cc(-n7c8ccccc8c8ccccc87)cc4c65)cc3)ccc21 ; CC(C)(C)c1ccc(-c2ccc3c(c2)B2c4ccc(-n5c6ccccc6c6cc(-c7ccccc7)ccc65)cc4N(c4ccc(-c5ccc6c(c5)C(C)(C)CC6(C)C)cc4)c4cc(-n5c6ccccc6c6ccccc65)cc(c42)N3c2ccc(-c3ccc4c(c3)C(C)(C)CC4(C)C)cc2)cc1]

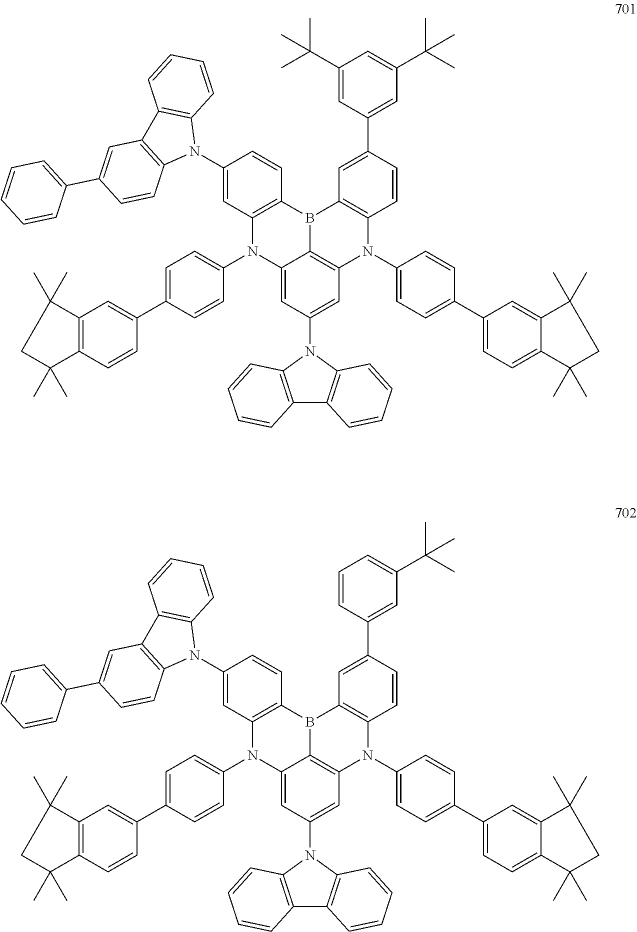 [SMILES: CC(C)(C)c1cc(-c2ccc3c(c2)B2c4ccc(-n5c6ccccc6c6cc(-c7ccccc7)ccc65)cc4N(c4ccc(-c5ccc6c(c5)C(C)(C)CC6(C)C)cc4)c4cc(-n5c6ccccc6c6ccccc65)cc(c42)N3c2ccc(-c3ccc4c(c3)C(C)(C)CC4(C)C)cc2)cc(C(C)(C)C)c1 ; CC(C)(C)c1cccc(-c2ccc3c(c2)B2c4ccc(-n5c6ccccc6c6cc(-c7ccccc7)ccc65)cc4N(c4ccc(-c5ccc6c(c5)C(C)(C)CC6(C)C)cc4)c4cc(-n5c6ccccc6c6ccccc65)cc(c42)N3c2ccc(-c3ccc4c(c3)C(C)(C)CC4(C)C)cc2)c1]

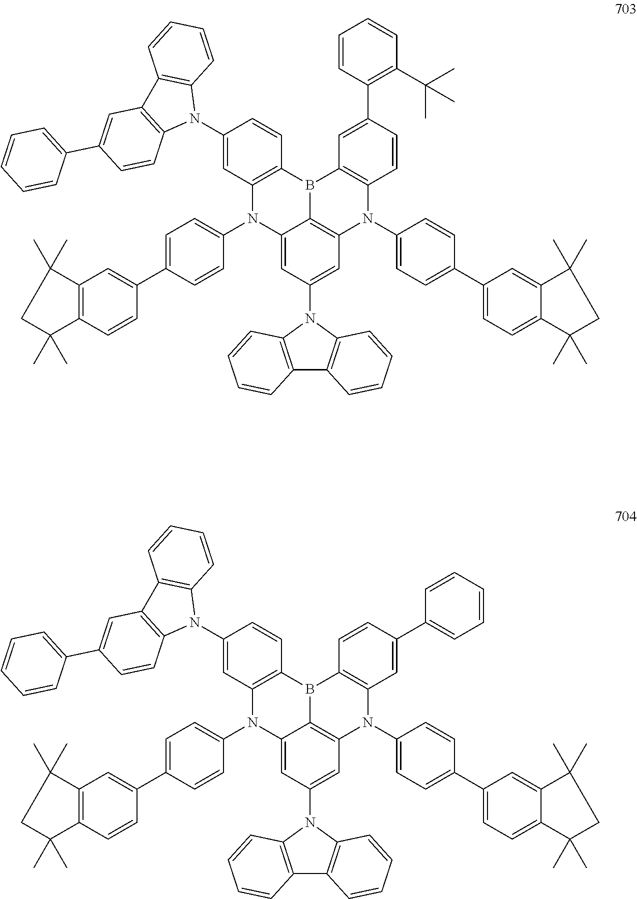 [SMILES: CC(C)(C)c1ccccc1-c1ccc2c(c1)B1c3ccc(-n4c5ccccc5c5cc(-c6ccccc6)ccc54)cc3N(c3ccc(-c4ccc5c(c4)C(C)(C)CC5(C)C)cc3)c3cc(-n4c5ccccc5c5ccccc54)cc(c31)N2c1ccc(-c2ccc3c(c2)C(C)(C)CC3(C)C)cc1 ; CC1(C)CC(C)(C)c2cc(-c3ccc(N4c5cc(-c6ccccc6)ccc5B5c6ccc(-n7c8ccccc8c8cc(-c9ccccc9)ccc87)cc6N(c6ccc(-c7ccc8c(c7)C(C)(C)CC8(C)C)cc6)c6cc(-n7c8ccccc8c8ccccc87)cc4c65)cc3)ccc21]

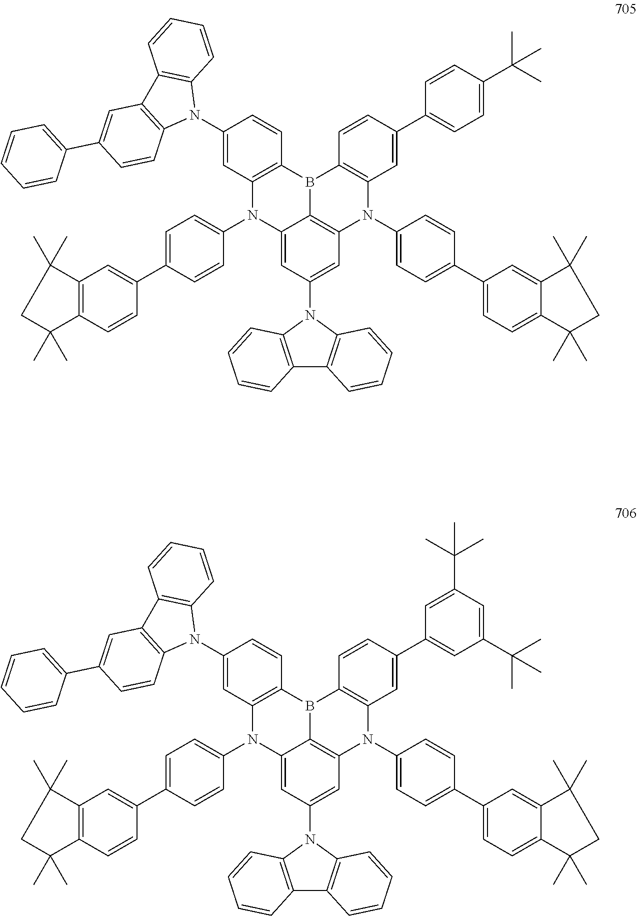 [SMILES: CC(C)(C)c1ccc(-c2ccc3c(c2)N(c2ccc(-c4ccc5c(c4)C(C)(C)CC5(C)C)cc2)c2cc(-n4c5ccccc5c5ccccc54)cc4c2B3c2ccc(-n3c5ccccc5c5cc(-c6ccccc6)ccc53)cc2N4c2ccc(-c3ccc4c(c3)C(C)(C)CC4(C)C)cc2)cc1 ; CC(C)(C)c1cc(-c2ccc3c(c2)N(c2ccc(-c4ccc5c(c4)C(C)(C)CC5(C)C)cc2)c2cc(-n4c5ccccc5c5ccccc54)cc4c2B3c2ccc(-n3c5ccccc5c5cc(-c6ccccc6)ccc53)cc2N4c2ccc(-c3ccc4c(c3)C(C)(C)CC4(C)C)cc2)cc(C(C)(C)C)c1]

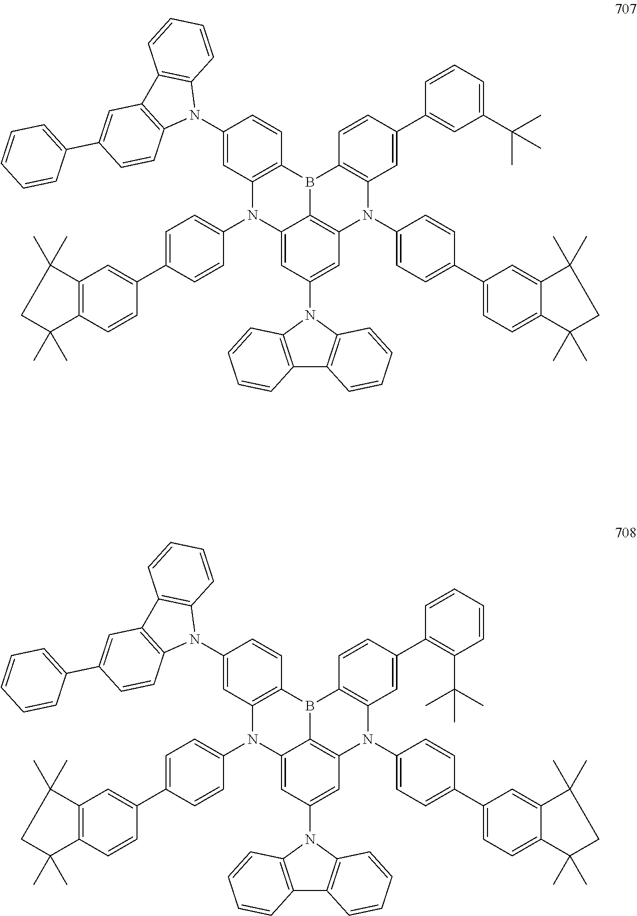 [SMILES: CC(C)(C)c1cccc(-c2ccc3c(c2)N(c2ccc(-c4ccc5c(c4)C(C)(C)CC5(C)C)cc2)c2cc(-n4c5ccccc5c5ccccc54)cc4c2B3c2ccc(-n3c5ccccc5c5cc(-c6ccccc6)ccc53)cc2N4c2ccc(-c3ccc4c(c3)C(C)(C)CC4(C)C)cc2)c1 ; CC(C)(C)c1ccccc1-c1ccc2c(c1)N(c1ccc(-c3ccc4c(c3)C(C)(C)CC4(C)C)cc1)c1cc(-n3c4ccccc4c4ccccc43)cc3c1B2c1ccc(-n2c4ccccc4c4cc(-c5ccccc5)ccc42)cc1N3c1ccc(-c2ccc3c(c2)C(C)(C)CC3(C)C)cc1]

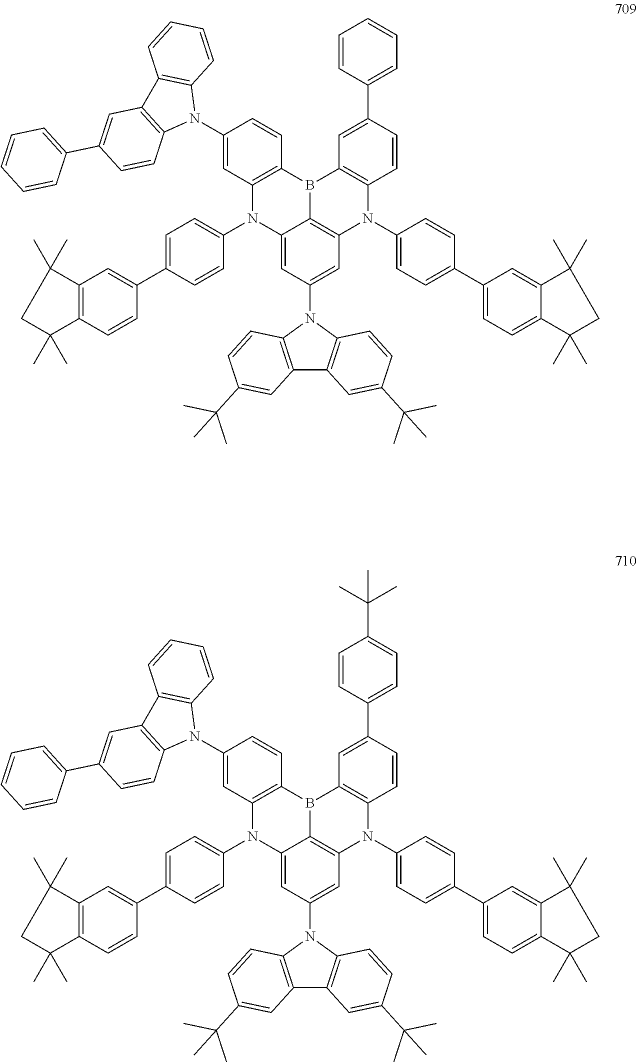 [SMILES: CC(C)(C)c1ccc2c(c1)c1cc(C(C)(C)C)ccc1n2-c1cc2c3c(c1)N(c1ccc(-c4ccc5c(c4)C(C)(C)CC5(C)C)cc1)c1cc(-n4c5ccccc5c5cc(-c6ccccc6)ccc54)ccc1B3c1cc(-c3ccccc3)ccc1N2c1ccc(-c2ccc3c(c2)C(C)(C)CC3(C)C)cc1 ; CC(C)(C)c1ccc(-c2ccc3c(c2)B2c4ccc(-n5c6ccccc6c6cc(-c7ccccc7)ccc65)cc4N(c4ccc(-c5ccc6c(c5)C(C)(C)CC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CC4(C)C)cc2)cc1]

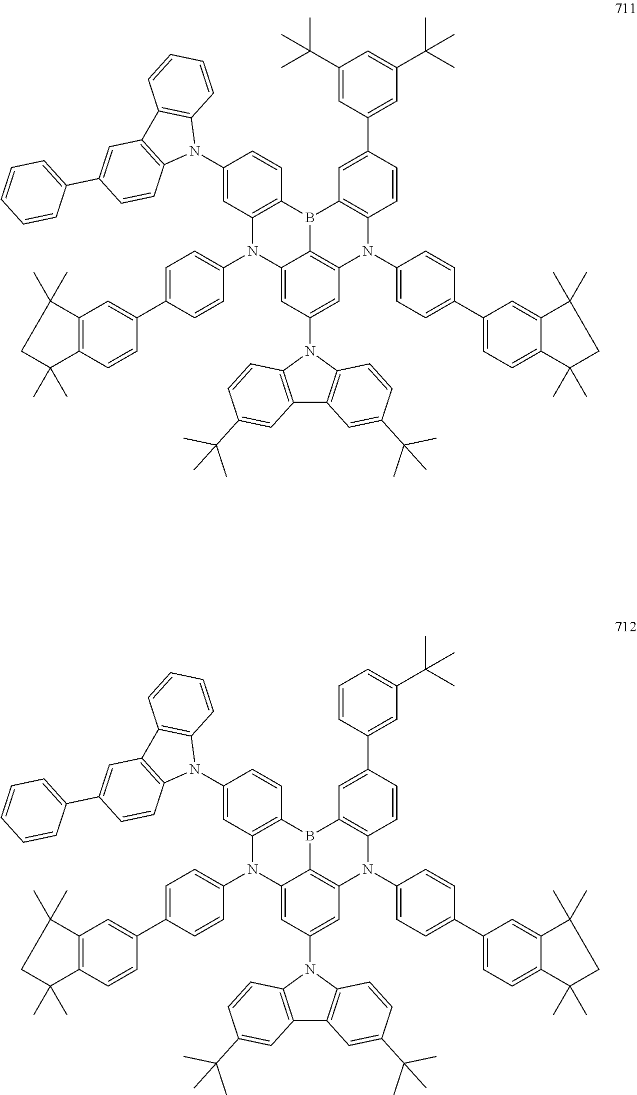 [SMILES: CC(C)(C)c1cc(-c2ccc3c(c2)B2c4ccc(-n5c6ccccc6c6cc(-c7ccccc7)ccc65)cc4N(c4ccc(-c5ccc6c(c5)C(C)(C)CC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CC4(C)C)cc2)cc(C(C)(C)C)c1 ; CC(C)(C)c1cccc(-c2ccc3c(c2)B2c4ccc(-n5c6ccccc6c6cc(-c7ccccc7)ccc65)cc4N(c4ccc(-c5ccc6c(c5)C(C)(C)CC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CC4(C)C)cc2)c1]

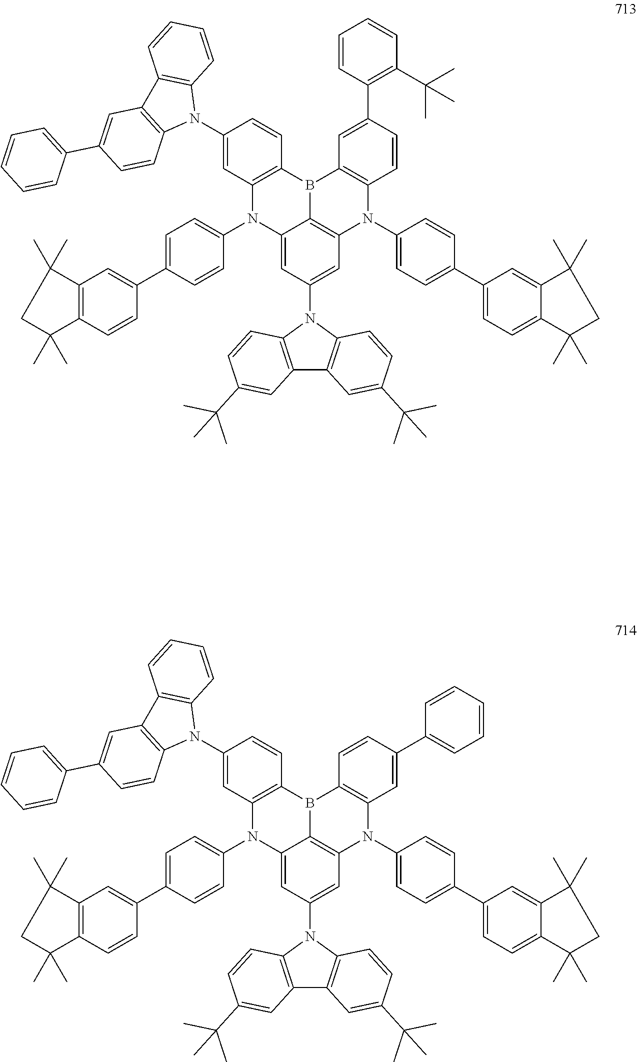 [SMILES: CC(C)(C)c1ccc2c(c1)c1cc(C(C)(C)C)ccc1n2-c1cc2c3c(c1)N(c1ccc(-c4ccc5c(c4)C(C)(C)CC5(C)C)cc1)c1cc(-n4c5ccccc5c5cc(-c6ccccc6)ccc54)ccc1B3c1cc(-c3ccccc3C(C)(C)C)ccc1N2c1ccc(-c2ccc3c(c2)C(C)(C)CC3(C)C)cc1 ; CC(C)(C)c1ccc2c(c1)c1cc(C(C)(C)C)ccc1n2-c1cc2c3c(c1)N(c1ccc(-c4ccc5c(c4)C(C)(C)CC5(C)C)cc1)c1cc(-n4c5ccccc5c5cc(-c6ccccc6)ccc54)ccc1B3c1ccc(-c3ccccc3)cc1N2c1ccc(-c2ccc3c(c2)C(C)(C)CC3(C)C)cc1]

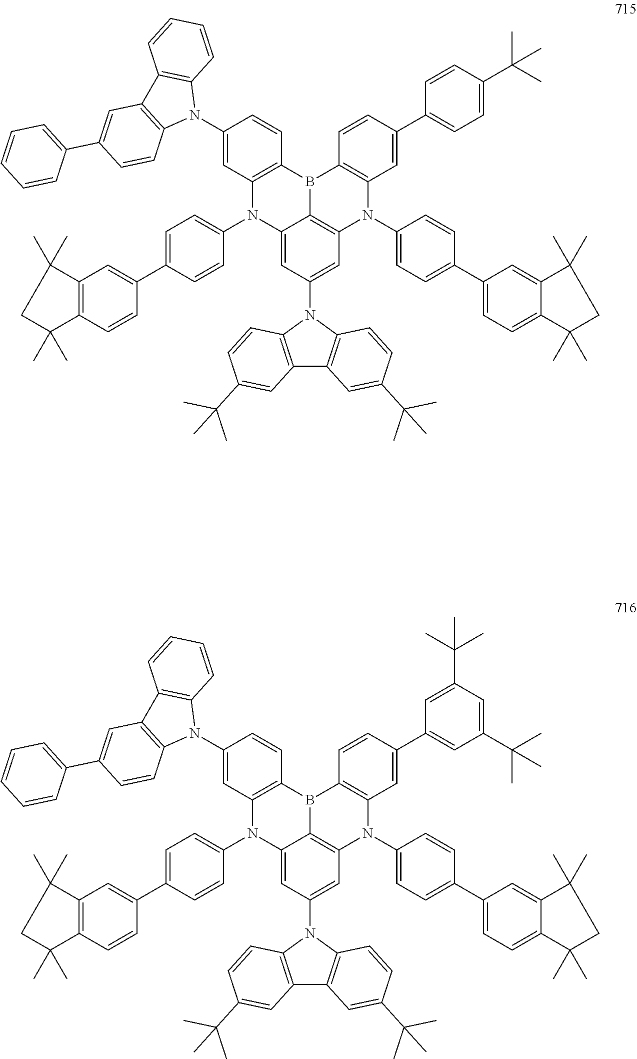 [SMILES: CC(C)(C)c1ccc(-c2ccc3c(c2)N(c2ccc(-c4ccc5c(c4)C(C)(C)CC5(C)C)cc2)c2cc(-n4c5ccc(C(C)(C)C)cc5c5cc(C(C)(C)C)ccc54)cc4c2B3c2ccc(-n3c5ccccc5c5cc(-c6ccccc6)ccc53)cc2N4c2ccc(-c3ccc4c(c3)C(C)(C)CC4(C)C)cc2)cc1 ; CC(C)(C)c1cc(-c2ccc3c(c2)N(c2ccc(-c4ccc5c(c4)C(C)(C)CC5(C)C)cc2)c2cc(-n4c5ccc(C(C)(C)C)cc5c5cc(C(C)(C)C)ccc54)cc4c2B3c2ccc(-n3c5ccccc5c5cc(-c6ccccc6)ccc53)cc2N4c2ccc(-c3ccc4c(c3)C(C)(C)CC4(C)C)cc2)cc(C(C)(C)C)c1]

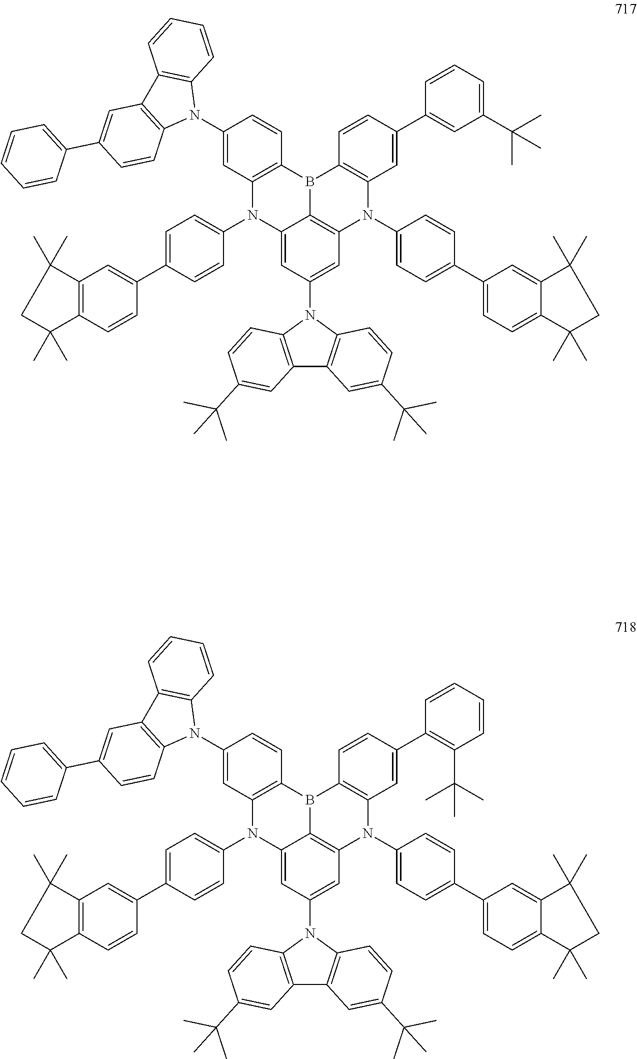 [SMILES: CC(C)(C)c1cccc(-c2ccc3c(c2)N(c2ccc(-c4ccc5c(c4)C(C)(C)CC5(C)C)cc2)c2cc(-n4c5ccc(C(C)(C)C)cc5c5cc(C(C)(C)C)ccc54)cc4c2B3c2ccc(-n3c5ccccc5c5cc(-c6ccccc6)ccc53)cc2N4c2ccc(-c3ccc4c(c3)C(C)(C)CC4(C)C)cc2)c1 ; CC(C)(C)c1ccc2c(c1)c1cc(C(C)(C)C)ccc1n2-c1cc2c3c(c1)N(c1ccc(-c4ccc5c(c4)C(C)(C)CC5(C)C)cc1)c1cc(-n4c5ccccc5c5cc(-c6ccccc6)ccc54)ccc1B3c1ccc(-c3ccccc3C(C)(C)C)cc1N2c1ccc(-c2ccc3c(c2)C(C)(C)CC3(C)C)cc1]

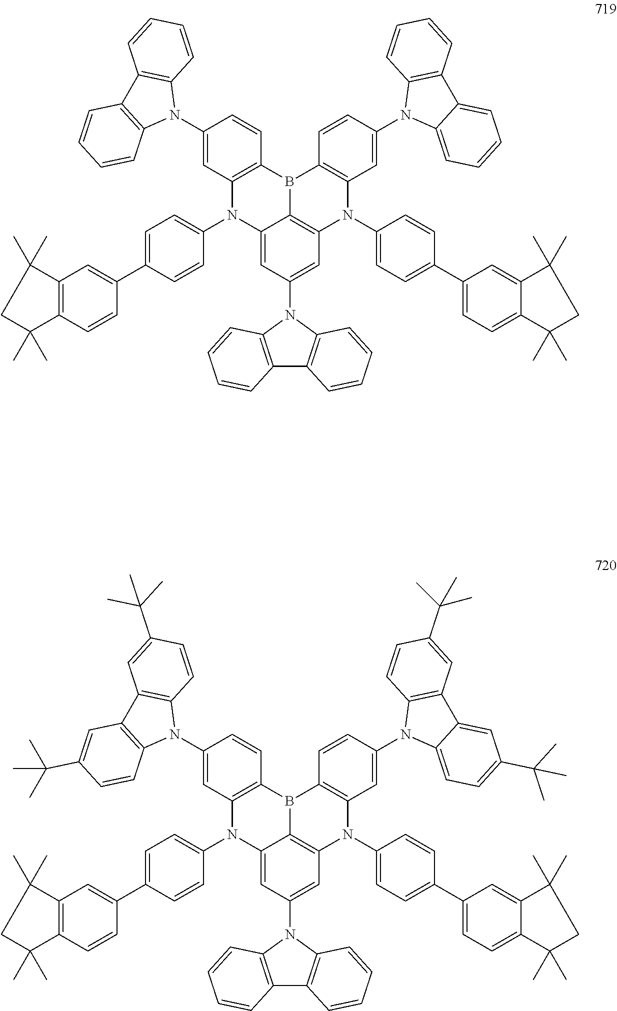 [SMILES: CC1(C)CC(C)(C)c2cc(-c3ccc(N4c5cc(-n6c7ccccc7c7ccccc76)ccc5B5c6ccc(-n7c8ccccc8c8ccccc87)cc6N(c6ccc(-c7ccc8c(c7)C(C)(C)CC8(C)C)cc6)c6cc(-n7c8ccccc8c8ccccc87)cc4c65)cc3)ccc21 ; CC(C)(C)c1ccc2c(c1)c1cc(C(C)(C)C)ccc1n2-c1ccc2c(c1)N(c1ccc(-c3ccc4c(c3)C(C)(C)CC4(C)C)cc1)c1cc(-n3c4ccccc4c4ccccc43)cc3c1B2c1ccc(-n2c4ccc(C(C)(C)C)cc4c4cc(C(C)(C)C)ccc42)cc1N3c1ccc(-c2ccc3c(c2)C(C)(C)CC3(C)C)cc1]

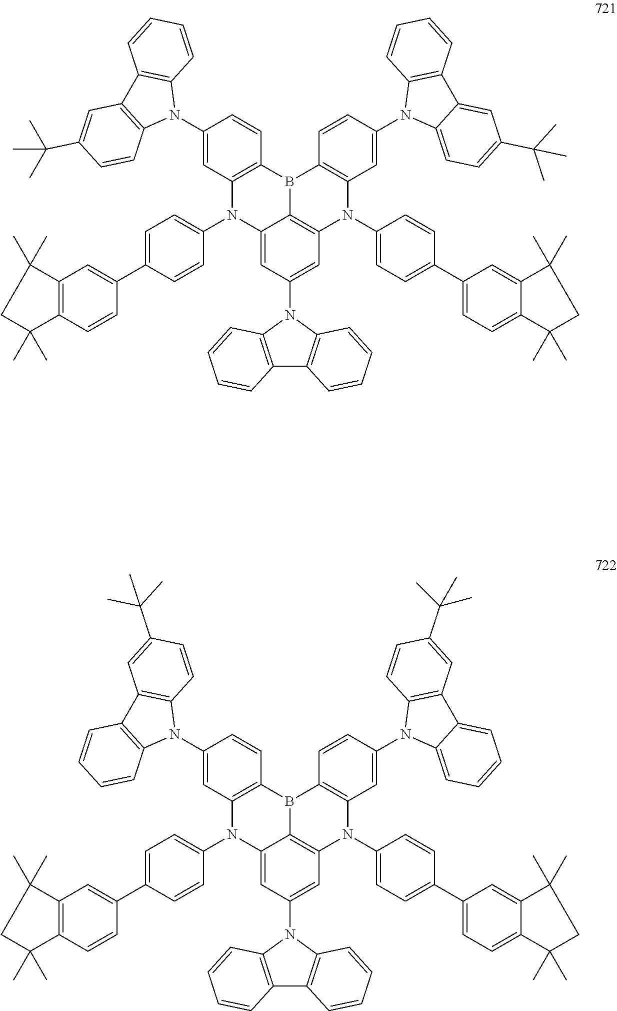 [SMILES: CC(C)(C)c1ccc2c(c1)c1ccccc1n2-c1ccc2c(c1)N(c1ccc(-c3ccc4c(c3)C(C)(C)CC4(C)C)cc1)c1cc(-n3c4ccccc4c4ccccc43)cc3c1B2c1ccc(-n2c4ccccc4c4cc(C(C)(C)C)ccc42)cc1N3c1ccc(-c2ccc3c(c2)C(C)(C)CC3(C)C)cc1 ; CC(C)(C)c1ccc2c(c1)c1ccccc1n2-c1ccc2c(c1)N(c1ccc(-c3ccc4c(c3)C(C)(C)CC4(C)C)cc1)c1cc(-n3c4ccccc4c4ccccc43)cc3c1B2c1ccc(-n2c4ccccc4c4cc(C(C)(C)C)ccc42)cc1N3c1ccc(-c2ccc3c(c2)C(C)(C)CC3(C)C)cc1]

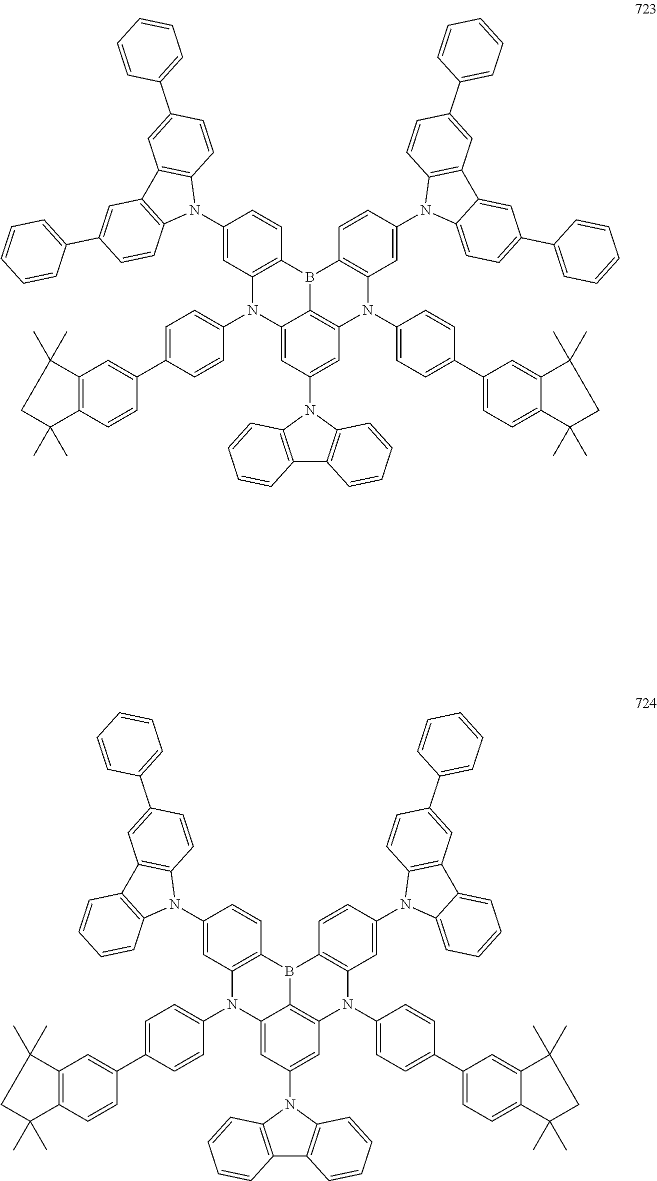 [SMILES: CC1(C)CC(C)(C)c2cc(-c3ccc(N4c5cc(-n6c7ccc(-c8ccccc8)cc7c7cc(-c8ccccc8)ccc76)ccc5B5c6ccc(-n7c8ccc(-c9ccccc9)cc8c8cc(-c9ccccc9)ccc87)cc6N(c6ccc(-c7ccc8c(c7)C(C)(C)CC8(C)C)cc6)c6cc(-n7c8ccccc8c8ccccc87)cc4c65)cc3)ccc21 ; CC1(C)CC(C)(C)c2cc(-c3ccc(N4c5cc(-n6c7ccccc7c7cc(-c8ccccc8)ccc76)ccc5B5c6ccc(-n7c8ccccc8c8cc(-c9ccccc9)ccc87)cc6N(c6ccc(-c7ccc8c(c7)C(C)(C)CC8(C)C)cc6)c6cc(-n7c8ccccc8c8ccccc87)cc4c65)cc3)ccc21]

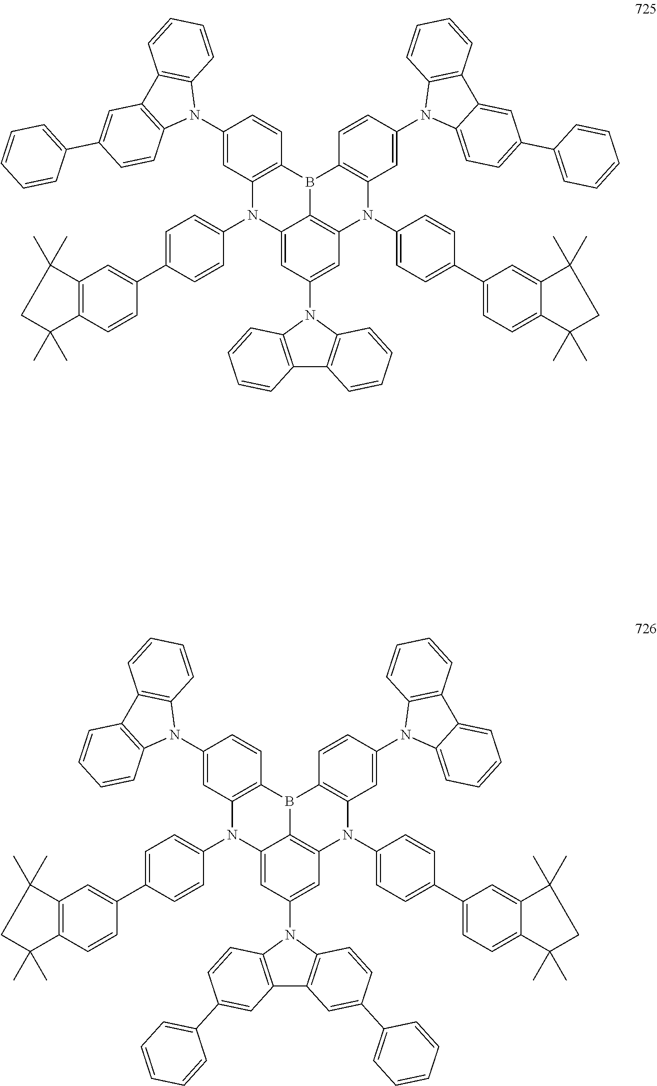 [SMILES: CC1(C)CC(C)(C)c2cc(-c3ccc(N4c5cc(-n6c7ccccc7c7cc(-c8ccccc8)ccc76)ccc5B5c6ccc(-n7c8ccccc8c8cc(-c9ccccc9)ccc87)cc6N(c6ccc(-c7ccc8c(c7)C(C)(C)CC8(C)C)cc6)c6cc(-n7c8ccccc8c8ccccc87)cc4c65)cc3)ccc21 ; CC1(C)CC(C)(C)c2cc(-c3ccc(N4c5cc(-n6c7ccccc7c7ccccc76)ccc5B5c6ccc(-n7c8ccccc8c8ccccc87)cc6N(c6ccc(-c7ccc8c(c7)C(C)(C)CC8(C)C)cc6)c6cc(-n7c8ccc(-c9ccccc9)cc8c8cc(-c9ccccc9)ccc87)cc4c65)cc3)ccc21]

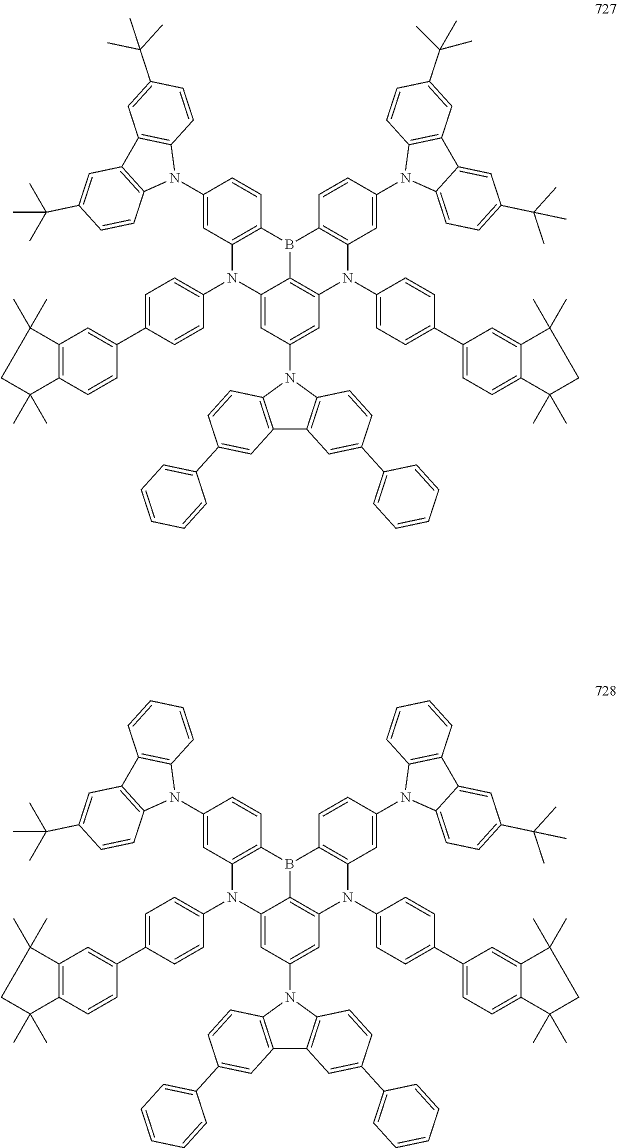 [SMILES: CC(C)(C)c1ccc2c(c1)c1cc(C(C)(C)C)ccc1n2-c1ccc2c(c1)N(c1ccc(-c3ccc4c(c3)C(C)(C)CC4(C)C)cc1)c1cc(-n3c4ccc(-c5ccccc5)cc4c4cc(-c5ccccc5)ccc43)cc3c1B2c1ccc(-n2c4ccc(C(C)(C)C)cc4c4cc(C(C)(C)C)ccc42)cc1N3c1ccc(-c2ccc3c(c2)C(C)(C)CC3(C)C)cc1 ; CC(C)(C)c1ccc2c(c1)c1ccccc1n2-c1ccc2c(c1)N(c1ccc(-c3ccc4c(c3)C(C)(C)CC4(C)C)cc1)c1cc(-n3c4ccc(-c5ccccc5)cc4c4cc(-c5ccccc5)ccc43)cc3c1B2c1ccc(-n2c4ccccc4c4cc(C(C)(C)C)ccc42)cc1N3c1ccc(-c2ccc3c(c2)C(C)(C)CC3(C)C)cc1]

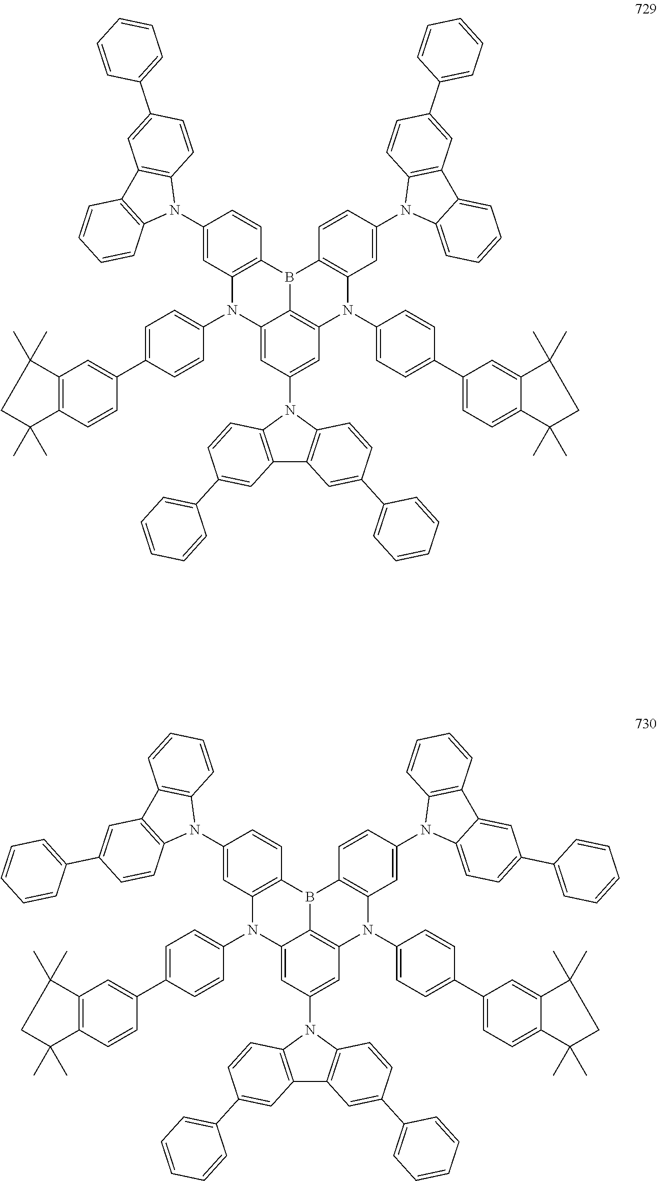 [SMILES: CC1(C)CC(C)(C)c2cc(-c3ccc(N4c5cc(-n6c7ccccc7c7cc(-c8ccccc8)ccc76)ccc5B5c6ccc(-n7c8ccccc8c8cc(-c9ccccc9)ccc87)cc6N(c6ccc(-c7ccc8c(c7)C(C)(C)CC8(C)C)cc6)c6cc(-n7c8ccc(-c9ccccc9)cc8c8cc(-c9ccccc9)ccc87)cc4c65)cc3)ccc21 ; CC1(C)CC(C)(C)c2cc(-c3ccc(N4c5cc(-n6c7ccccc7c7cc(-c8ccccc8)ccc76)ccc5B5c6ccc(-n7c8ccccc8c8cc(-c9ccccc9)ccc87)cc6N(c6ccc(-c7ccc8c(c7)C(C)(C)CC8(C)C)cc6)c6cc(-n7c8ccc(-c9ccccc9)cc8c8cc(-c9ccccc9)ccc87)cc4c65)cc3)ccc21]

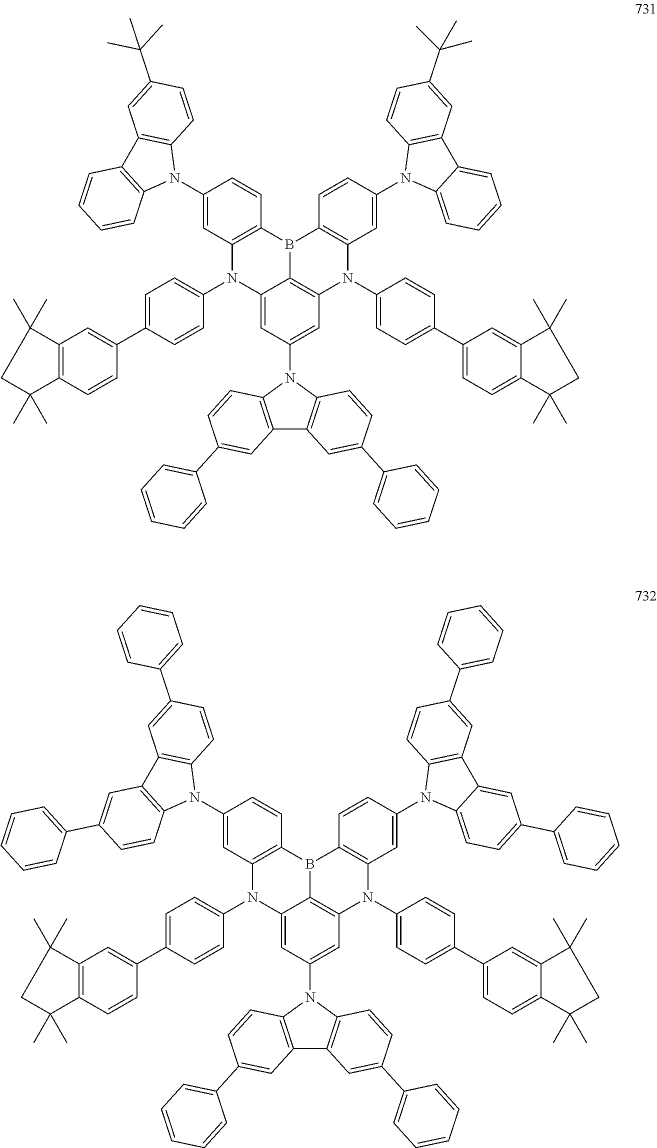 [SMILES: CC(C)(C)c1ccc2c(c1)c1ccccc1n2-c1ccc2c(c1)N(c1ccc(-c3ccc4c(c3)C(C)(C)CC4(C)C)cc1)c1cc(-n3c4ccc(-c5ccccc5)cc4c4cc(-c5ccccc5)ccc43)cc3c1B2c1ccc(-n2c4ccccc4c4cc(C(C)(C)C)ccc42)cc1N3c1ccc(-c2ccc3c(c2)C(C)(C)CC3(C)C)cc1 ; CC1(C)CC(C)(C)c2cc(-c3ccc(N4c5cc(-n6c7ccc(-c8ccccc8)cc7c7cc(-c8ccccc8)ccc76)ccc5B5c6ccc(-n7c8ccc(-c9ccccc9)cc8c8cc(-c9ccccc9)ccc87)cc6N(c6ccc(-c7ccc8c(c7)C(C)(C)CC8(C)C)cc6)c6cc(-n7c8ccc(-c9ccccc9)cc8c8cc(-c9ccccc9)ccc87)cc4c65)cc3)ccc21]

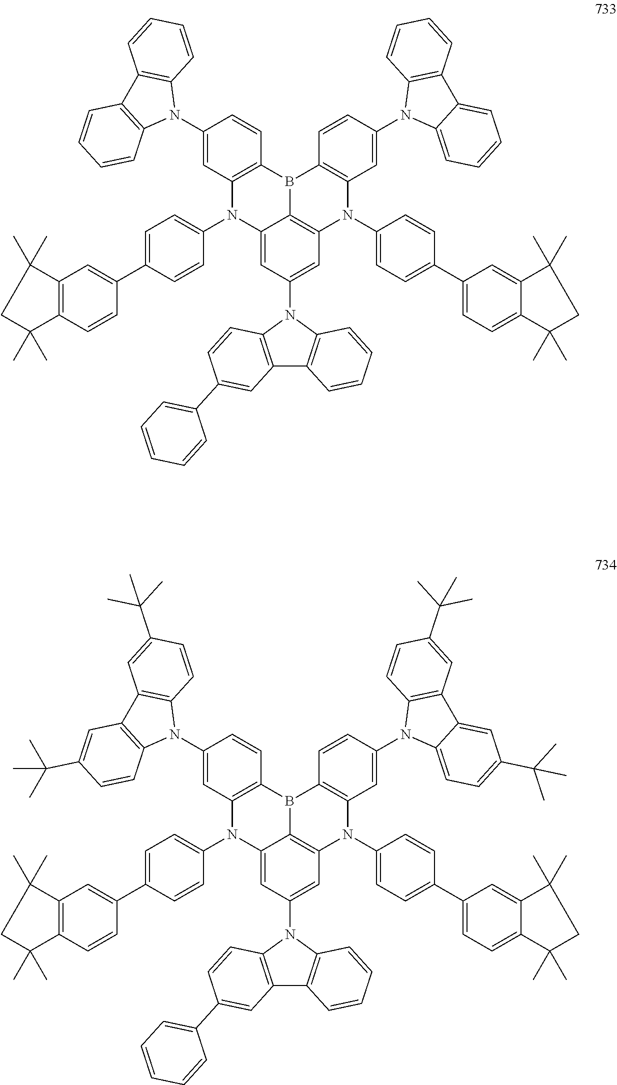 [SMILES: CC1(C)CC(C)(C)c2cc(-c3ccc(N4c5cc(-n6c7ccccc7c7ccccc76)ccc5B5c6ccc(-n7c8ccccc8c8ccccc87)cc6N(c6ccc(-c7ccc8c(c7)C(C)(C)CC8(C)C)cc6)c6cc(-n7c8ccccc8c8cc(-c9ccccc9)ccc87)cc4c65)cc3)ccc21 ; CC(C)(C)c1ccc2c(c1)c1cc(C(C)(C)C)ccc1n2-c1ccc2c(c1)N(c1ccc(-c3ccc4c(c3)C(C)(C)CC4(C)C)cc1)c1cc(-n3c4ccccc4c4cc(-c5ccccc5)ccc43)cc3c1B2c1ccc(-n2c4ccc(C(C)(C)C)cc4c4cc(C(C)(C)C)ccc42)cc1N3c1ccc(-c2ccc3c(c2)C(C)(C)CC3(C)C)cc1]

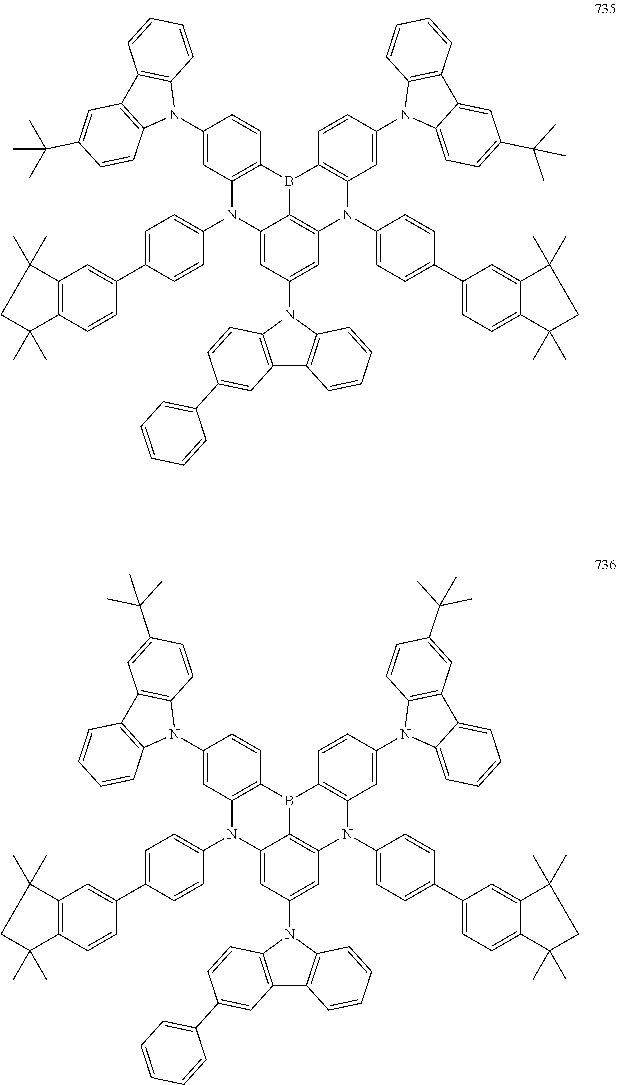 [SMILES: CC(C)(C)c1ccc2c(c1)c1ccccc1n2-c1ccc2c(c1)N(c1ccc(-c3ccc4c(c3)C(C)(C)CC4(C)C)cc1)c1cc(-n3c4ccccc4c4cc(-c5ccccc5)ccc43)cc3c1B2c1ccc(-n2c4ccccc4c4cc(C(C)(C)C)ccc42)cc1N3c1ccc(-c2ccc3c(c2)C(C)(C)CC3(C)C)cc1 ; CC(C)(C)c1ccc2c(c1)c1ccccc1n2-c1ccc2c(c1)N(c1ccc(-c3ccc4c(c3)C(C)(C)CC4(C)C)cc1)c1cc(-n3c4ccccc4c4cc(-c5ccccc5)ccc43)cc3c1B2c1ccc(-n2c4ccccc4c4cc(C(C)(C)C)ccc42)cc1N3c1ccc(-c2ccc3c(c2)C(C)(C)CC3(C)C)cc1]

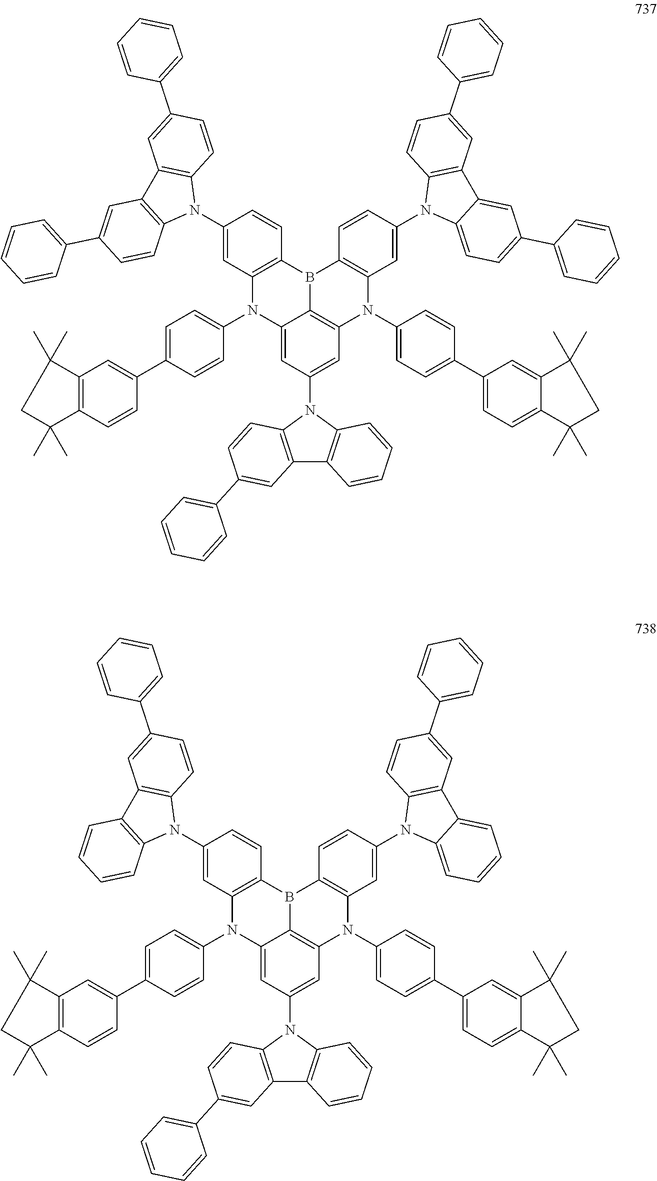 [SMILES: CC1(C)CC(C)(C)c2cc(-c3ccc(N4c5cc(-n6c7ccc(-c8ccccc8)cc7c7cc(-c8ccccc8)ccc76)ccc5B5c6ccc(-n7c8ccc(-c9ccccc9)cc8c8cc(-c9ccccc9)ccc87)cc6N(c6ccc(-c7ccc8c(c7)C(C)(C)CC8(C)C)cc6)c6cc(-n7c8ccccc8c8cc(-c9ccccc9)ccc87)cc4c65)cc3)ccc21 ; CC1(C)CC(C)(C)c2cc(-c3ccc(N4c5cc(-n6c7ccccc7c7cc(-c8ccccc8)ccc76)ccc5B5c6ccc(-n7c8ccccc8c8cc(-c9ccccc9)ccc87)cc6N(c6ccc(-c7ccc8c(c7)C(C)(C)CC8(C)C)cc6)c6cc(-n7c8ccccc8c8cc(-c9ccccc9)ccc87)cc4c65)cc3)ccc21]

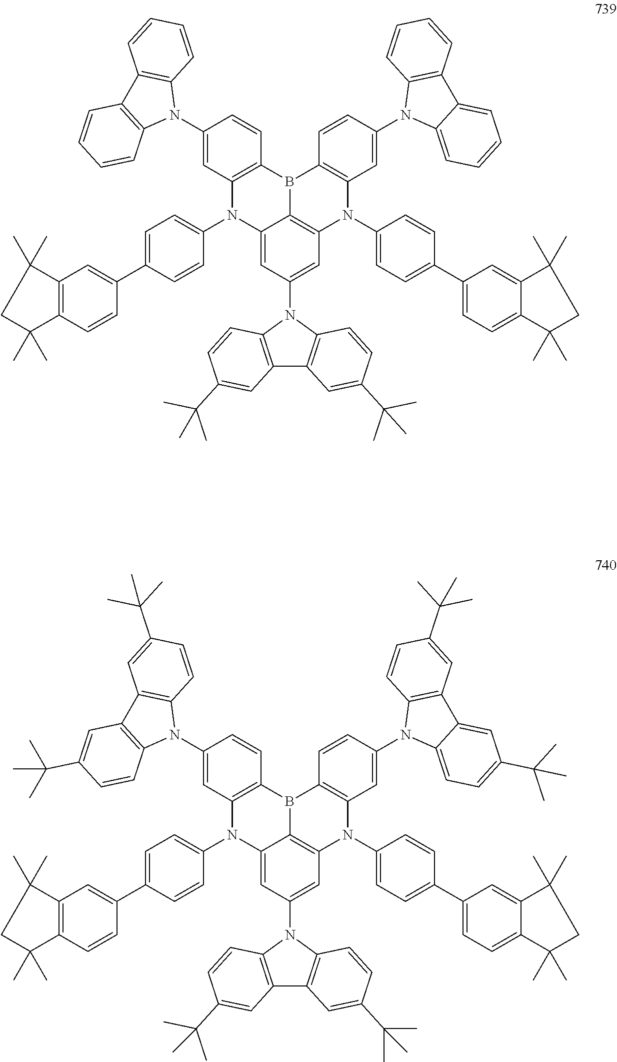 [SMILES: CC(C)(C)c1ccc2c(c1)c1cc(C(C)(C)C)ccc1n2-c1cc2c3c(c1)N(c1ccc(-c4ccc5c(c4)C(C)(C)CC5(C)C)cc1)c1cc(-n4c5ccccc5c5ccccc54)ccc1B3c1ccc(-n3c4ccccc4c4ccccc43)cc1N2c1ccc(-c2ccc3c(c2)C(C)(C)CC3(C)C)cc1 ; CC(C)(C)c1ccc2c(c1)c1cc(C(C)(C)C)ccc1n2-c1ccc2c(c1)N(c1ccc(-c3ccc4c(c3)C(C)(C)CC4(C)C)cc1)c1cc(-n3c4ccc(C(C)(C)C)cc4c4cc(C(C)(C)C)ccc43)cc3c1B2c1ccc(-n2c4ccc(C(C)(C)C)cc4c4cc(C(C)(C)C)ccc42)cc1N3c1ccc(-c2ccc3c(c2)C(C)(C)CC3(C)C)cc1]

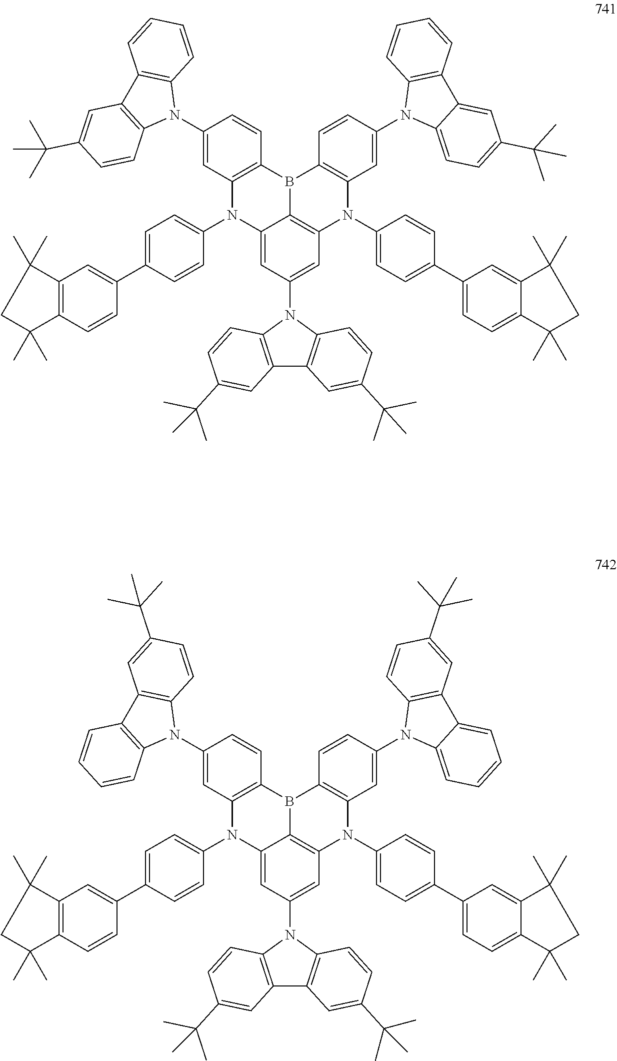 [SMILES: CC(C)(C)c1ccc2c(c1)c1ccccc1n2-c1ccc2c(c1)N(c1ccc(-c3ccc4c(c3)C(C)(C)CC4(C)C)cc1)c1cc(-n3c4ccc(C(C)(C)C)cc4c4cc(C(C)(C)C)ccc43)cc3c1B2c1ccc(-n2c4ccccc4c4cc(C(C)(C)C)ccc42)cc1N3c1ccc(-c2ccc3c(c2)C(C)(C)CC3(C)C)cc1 ; CC(C)(C)c1ccc2c(c1)c1ccccc1n2-c1ccc2c(c1)N(c1ccc(-c3ccc4c(c3)C(C)(C)CC4(C)C)cc1)c1cc(-n3c4ccc(C(C)(C)C)cc4c4cc(C(C)(C)C)ccc43)cc3c1B2c1ccc(-n2c4ccccc4c4cc(C(C)(C)C)ccc42)cc1N3c1ccc(-c2ccc3c(c2)C(C)(C)CC3(C)C)cc1]

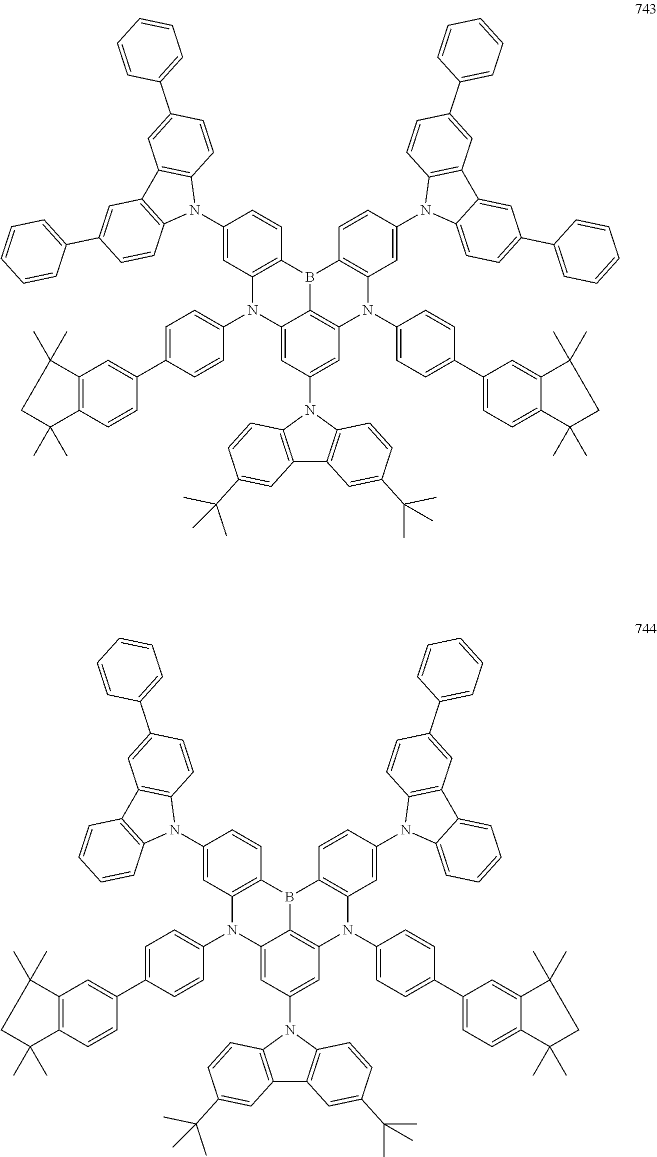 [SMILES: CC(C)(C)c1ccc2c(c1)c1cc(C(C)(C)C)ccc1n2-c1cc2c3c(c1)N(c1ccc(-c4ccc5c(c4)C(C)(C)CC5(C)C)cc1)c1cc(-n4c5ccc(-c6ccccc6)cc5c5cc(-c6ccccc6)ccc54)ccc1B3c1ccc(-n3c4ccc(-c5ccccc5)cc4c4cc(-c5ccccc5)ccc43)cc1N2c1ccc(-c2ccc3c(c2)C(C)(C)CC3(C)C)cc1 ; CC(C)(C)c1ccc2c(c1)c1cc(C(C)(C)C)ccc1n2-c1cc2c3c(c1)N(c1ccc(-c4ccc5c(c4)C(C)(C)CC5(C)C)cc1)c1cc(-n4c5ccccc5c5cc(-c6ccccc6)ccc54)ccc1B3c1ccc(-n3c4ccccc4c4cc(-c5ccccc5)ccc43)cc1N2c1ccc(-c2ccc3c(c2)C(C)(C)CC3(C)C)cc1]

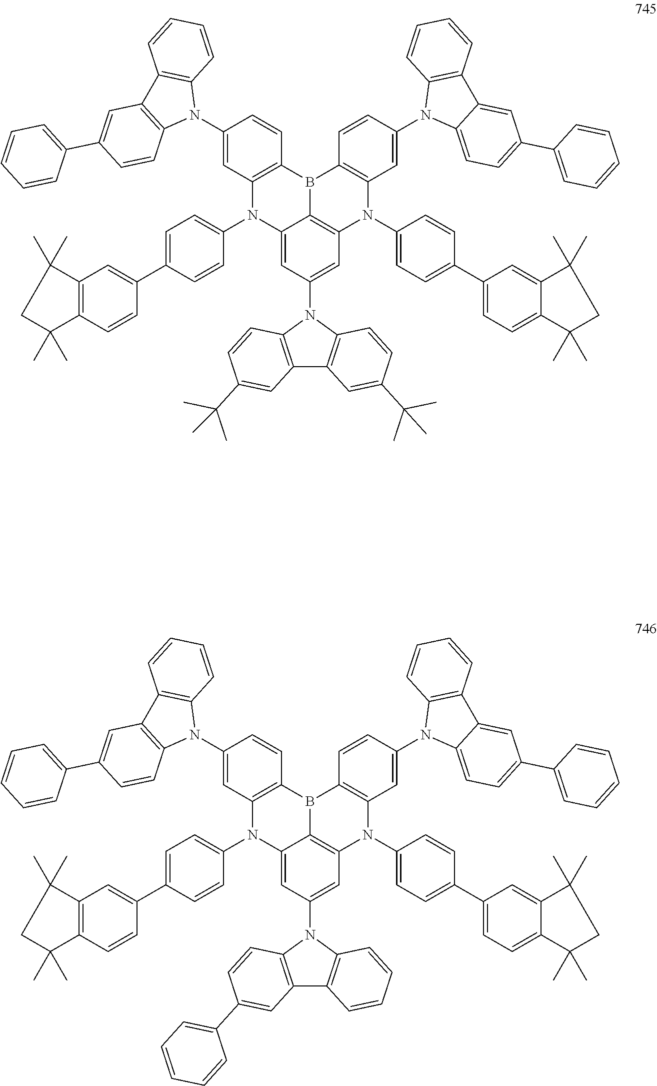 [SMILES: CC(C)(C)c1ccc2c(c1)c1cc(C(C)(C)C)ccc1n2-c1cc2c3c(c1)N(c1ccc(-c4ccc5c(c4)C(C)(C)CC5(C)C)cc1)c1cc(-n4c5ccccc5c5cc(-c6ccccc6)ccc54)ccc1B3c1ccc(-n3c4ccccc4c4cc(-c5ccccc5)ccc43)cc1N2c1ccc(-c2ccc3c(c2)C(C)(C)CC3(C)C)cc1 ; CC1(C)CC(C)(C)c2cc(-c3ccc(N4c5cc(-n6c7ccccc7c7cc(-c8ccccc8)ccc76)ccc5B5c6ccc(-n7c8ccccc8c8cc(-c9ccccc9)ccc87)cc6N(c6ccc(-c7ccc8c(c7)C(C)(C)CC8(C)C)cc6)c6cc(-n7c8ccccc8c8cc(-c9ccccc9)ccc87)cc4c65)cc3)ccc21]

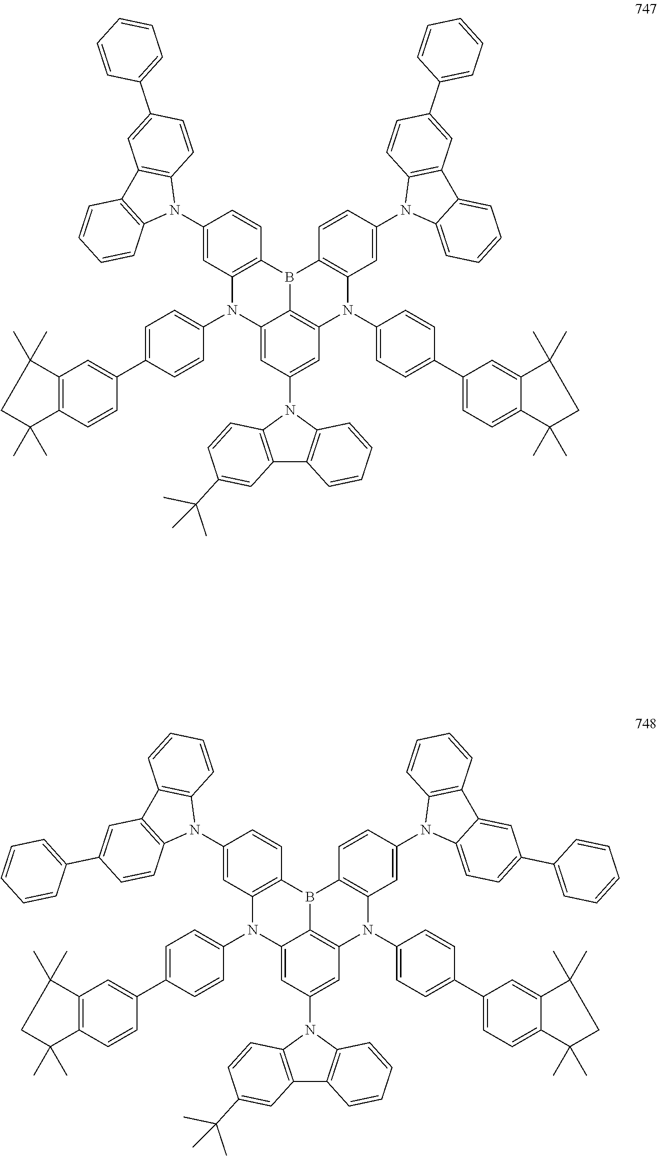 [SMILES: CC(C)(C)c1ccc2c(c1)c1ccccc1n2-c1cc2c3c(c1)N(c1ccc(-c4ccc5c(c4)C(C)(C)CC5(C)C)cc1)c1cc(-n4c5ccccc5c5cc(-c6ccccc6)ccc54)ccc1B3c1ccc(-n3c4ccccc4c4cc(-c5ccccc5)ccc43)cc1N2c1ccc(-c2ccc3c(c2)C(C)(C)CC3(C)C)cc1 ; CC(C)(C)c1ccc2c(c1)c1ccccc1n2-c1cc2c3c(c1)N(c1ccc(-c4ccc5c(c4)C(C)(C)CC5(C)C)cc1)c1cc(-n4c5ccccc5c5cc(-c6ccccc6)ccc54)ccc1B3c1ccc(-n3c4ccccc4c4cc(-c5ccccc5)ccc43)cc1N2c1ccc(-c2ccc3c(c2)C(C)(C)CC3(C)C)cc1]

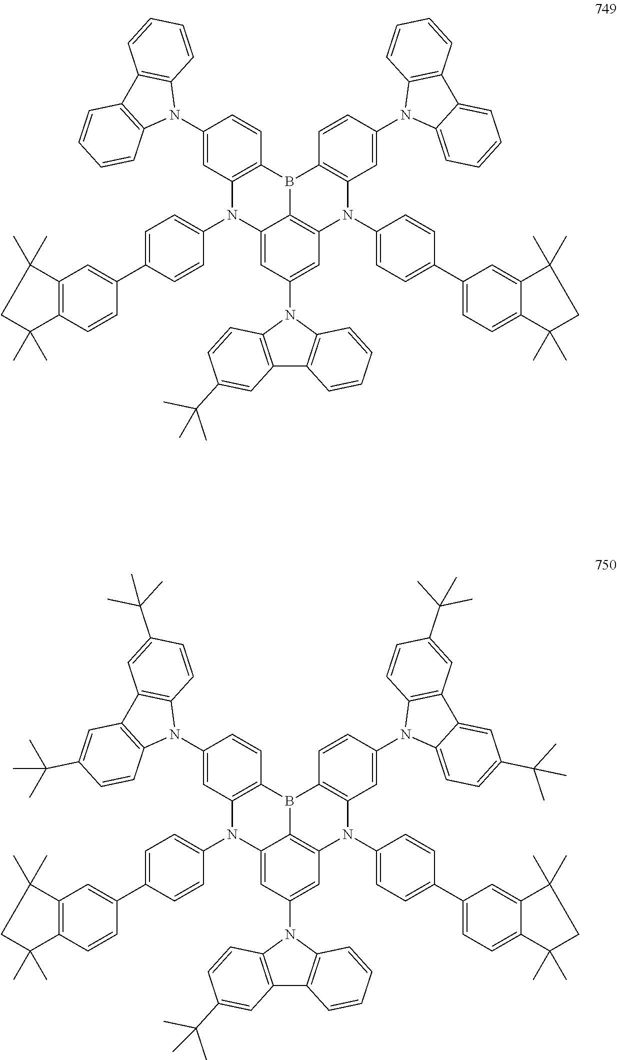 [SMILES: CC(C)(C)c1ccc2c(c1)c1ccccc1n2-c1cc2c3c(c1)N(c1ccc(-c4ccc5c(c4)C(C)(C)CC5(C)C)cc1)c1cc(-n4c5ccccc5c5ccccc54)ccc1B3c1ccc(-n3c4ccccc4c4ccccc43)cc1N2c1ccc(-c2ccc3c(c2)C(C)(C)CC3(C)C)cc1 ; CC(C)(C)c1ccc2c(c1)c1ccccc1n2-c1cc2c3c(c1)N(c1ccc(-c4ccc5c(c4)C(C)(C)CC5(C)C)cc1)c1cc(-n4c5ccc(C(C)(C)C)cc5c5cc(C(C)(C)C)ccc54)ccc1B3c1ccc(-n3c4ccc(C(C)(C)C)cc4c4cc(C(C)(C)C)ccc43)cc1N2c1ccc(-c2ccc3c(c2)C(C)(C)CC3(C)C)cc1]

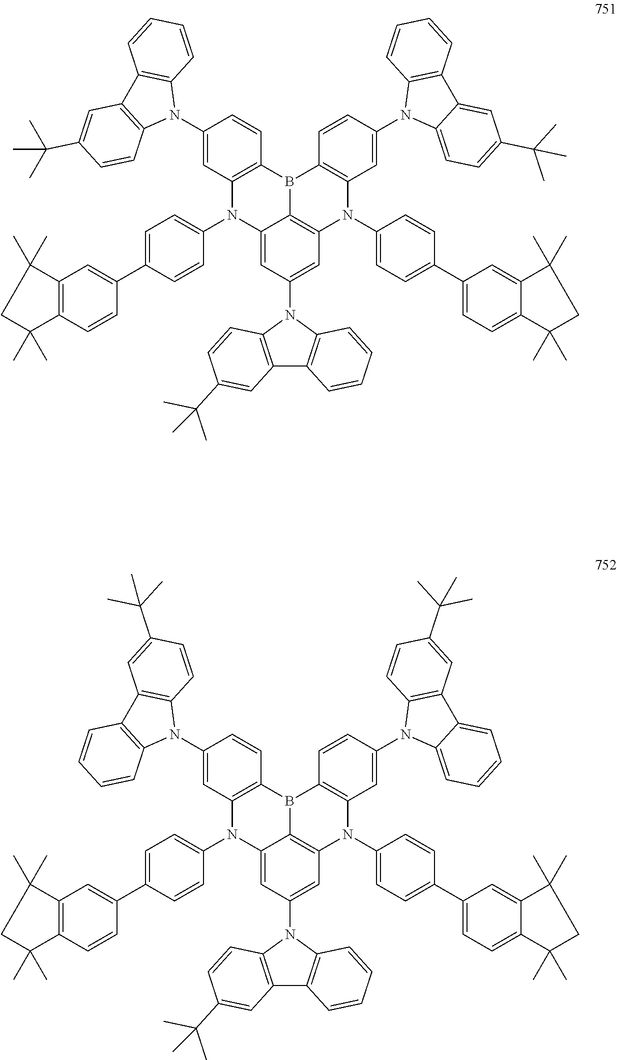 [SMILES: CC(C)(C)c1ccc2c(c1)c1ccccc1n2-c1ccc2c(c1)N(c1ccc(-c3ccc4c(c3)C(C)(C)CC4(C)C)cc1)c1cc(-n3c4ccccc4c4cc(C(C)(C)C)ccc43)cc3c1B2c1ccc(-n2c4ccccc4c4cc(C(C)(C)C)ccc42)cc1N3c1ccc(-c2ccc3c(c2)C(C)(C)CC3(C)C)cc1 ; CC(C)(C)c1ccc2c(c1)c1ccccc1n2-c1ccc2c(c1)N(c1ccc(-c3ccc4c(c3)C(C)(C)CC4(C)C)cc1)c1cc(-n3c4ccccc4c4cc(C(C)(C)C)ccc43)cc3c1B2c1ccc(-n2c4ccccc4c4cc(C(C)(C)C)ccc42)cc1N3c1ccc(-c2ccc3c(c2)C(C)(C)CC3(C)C)cc1]

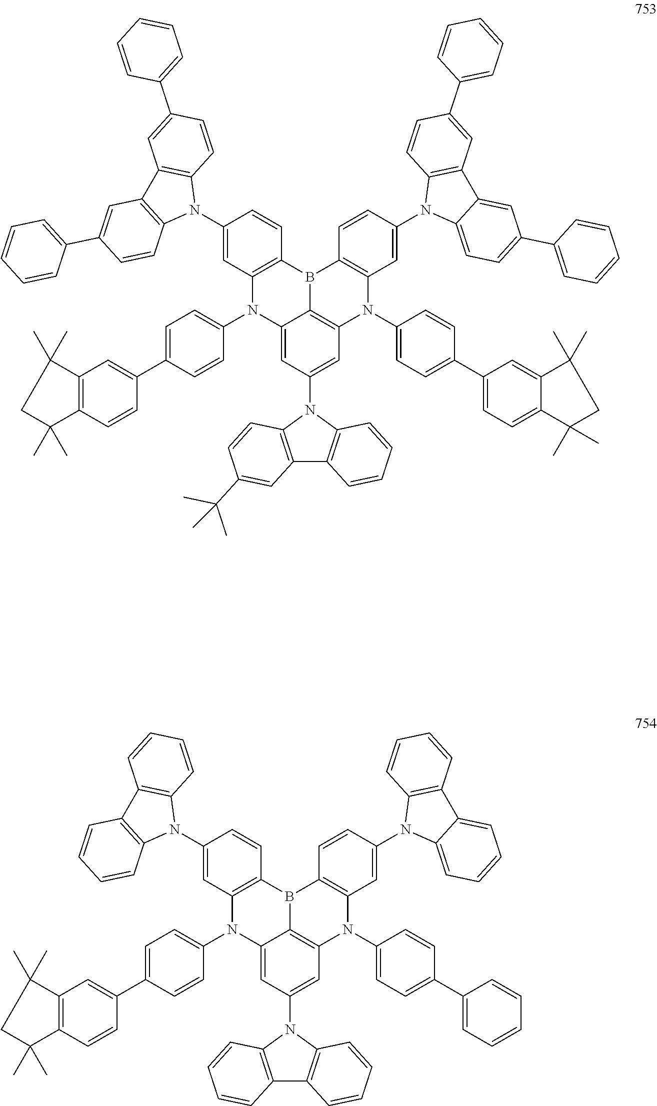 [SMILES: CC(C)(C)c1ccc2c(c1)c1ccccc1n2-c1cc2c3c(c1)N(c1ccc(-c4ccc5c(c4)C(C)(C)CC5(C)C)cc1)c1cc(-n4c5ccc(-c6ccccc6)cc5c5cc(-c6ccccc6)ccc54)ccc1B3c1ccc(-n3c4ccc(-c5ccccc5)cc4c4cc(-c5ccccc5)ccc43)cc1N2c1ccc(-c2ccc3c(c2)C(C)(C)CC3(C)C)cc1 ; CC1(C)CC(C)(C)c2cc(-c3ccc(N4c5cc(-n6c7ccccc7c7ccccc76)ccc5B5c6ccc(-n7c8ccccc8c8ccccc87)cc6N(c6ccc(-c7ccccc7)cc6)c6cc(-n7c8ccccc8c8ccccc87)cc4c65)cc3)ccc21]

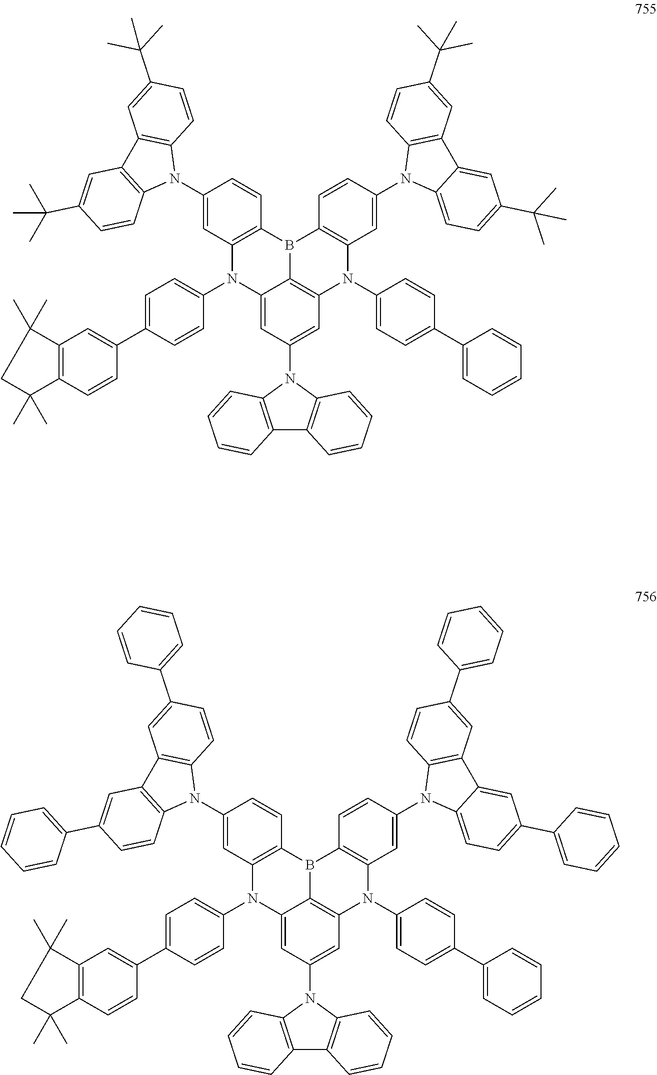 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1ccc(-n2c4ccc(C(C)(C)C)cc4c4cc(C(C)(C)C)ccc42)cc1N3c1ccc(-c2ccc3c(c2)C(C)(C)CC3(C)C)cc1 ; CC1(C)CC(C)(C)c2cc(-c3ccc(N4c5cc(-n6c7ccc(-c8ccccc8)cc7c7cc(-c8ccccc8)ccc76)ccc5B5c6ccc(-n7c8ccc(-c9ccccc9)cc8c8cc(-c9ccccc9)ccc87)cc6N(c6ccc(-c7ccccc7)cc6)c6cc(-n7c8ccccc8c8ccccc87)cc4c65)cc3)ccc21]

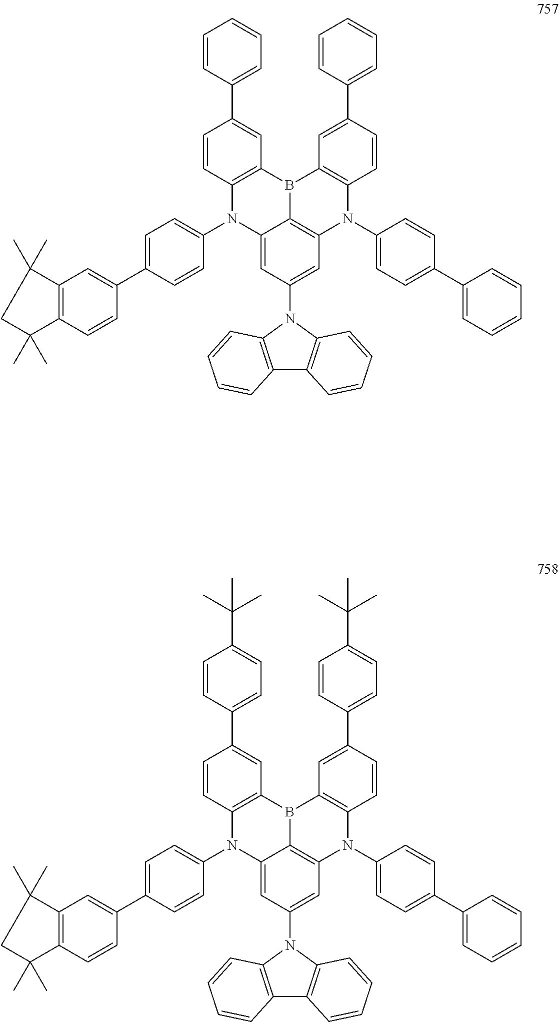 [SMILES: CC1(C)CC(C)(C)c2cc(-c3ccc(N4c5ccc(-c6ccccc6)cc5B5c6cc(-c7ccccc7)ccc6N(c6ccc(-c7ccccc7)cc6)c6cc(-n7c8ccccc8c8ccccc87)cc4c65)cc3)ccc21 ; CC(C)(C)c1ccc(-c2ccc3c(c2)B2c4cc(-c5ccc(C(C)(C)C)cc5)ccc4N(c4ccc(-c5ccc6c(c5)C(C)(C)CC6(C)C)cc4)c4cc(-n5c6ccccc6c6ccccc65)cc(c42)N3c2ccc(-c3ccccc3)cc2)cc1]

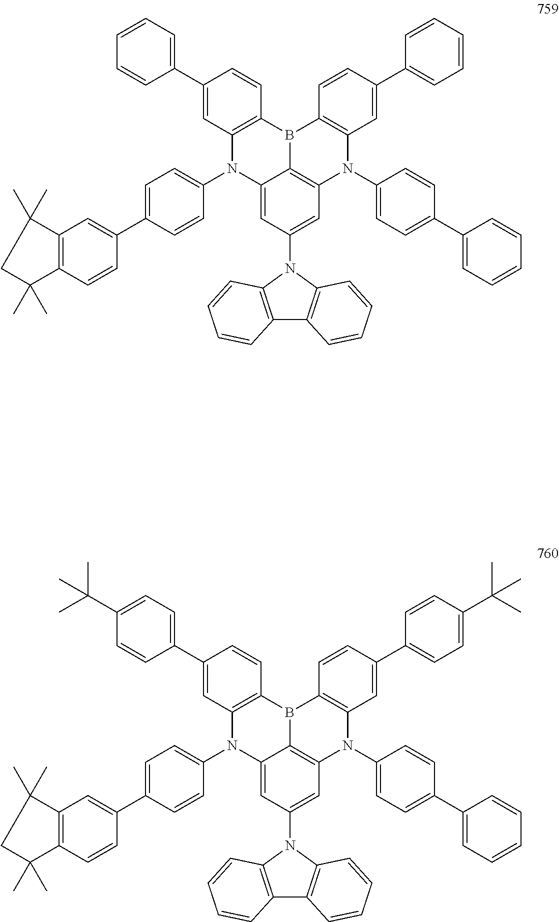 [SMILES: CC1(C)CC(C)(C)c2cc(-c3ccc(N4c5cc(-c6ccccc6)ccc5B5c6ccc(-c7ccccc7)cc6N(c6ccc(-c7ccccc7)cc6)c6cc(-n7c8ccccc8c8ccccc87)cc4c65)cc3)ccc21 ; CC(C)(C)c1ccc(-c2ccc3c(c2)N(c2ccc(-c4ccccc4)cc2)c2cc(-n4c5ccccc5c5ccccc54)cc4c2B3c2ccc(-c3ccc(C(C)(C)C)cc3)cc2N4c2ccc(-c3ccc4c(c3)C(C)(C)CC4(C)C)cc2)cc1]

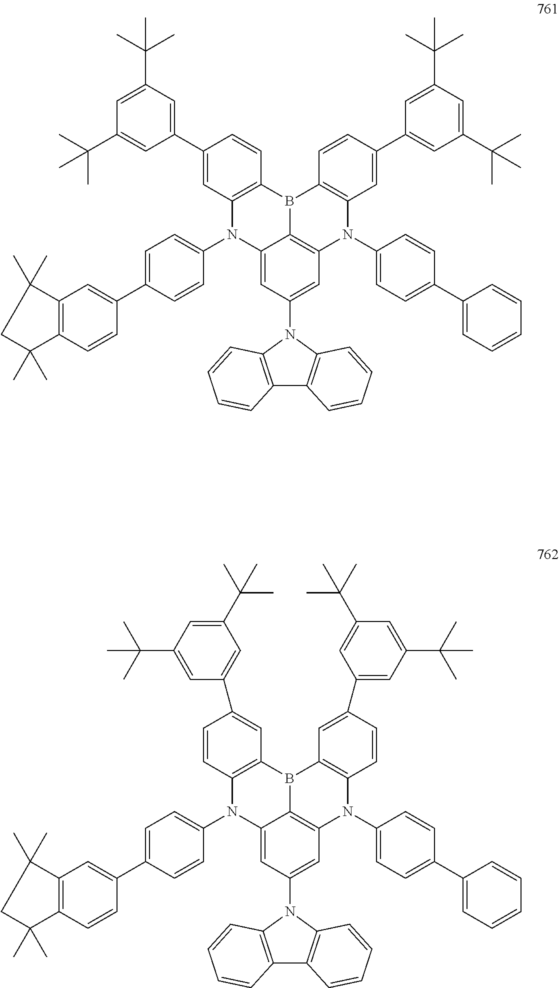 [SMILES: CC(C)(C)c1cc(-c2ccc3c(c2)N(c2ccc(-c4ccccc4)cc2)c2cc(-n4c5ccccc5c5ccccc54)cc4c2B3c2ccc(-c3cc(C(C)(C)C)cc(C(C)(C)C)c3)cc2N4c2ccc(-c3ccc4c(c3)C(C)(C)CC4(C)C)cc2)cc(C(C)(C)C)c1 ; CC(C)(C)c1cc(-c2ccc3c(c2)B2c4cc(-c5cc(C(C)(C)C)cc(C(C)(C)C)c5)ccc4N(c4ccc(-c5ccc6c(c5)C(C)(C)CC6(C)C)cc4)c4cc(-n5c6ccccc6c6ccccc65)cc(c42)N3c2ccc(-c3ccccc3)cc2)cc(C(C)(C)C)c1]

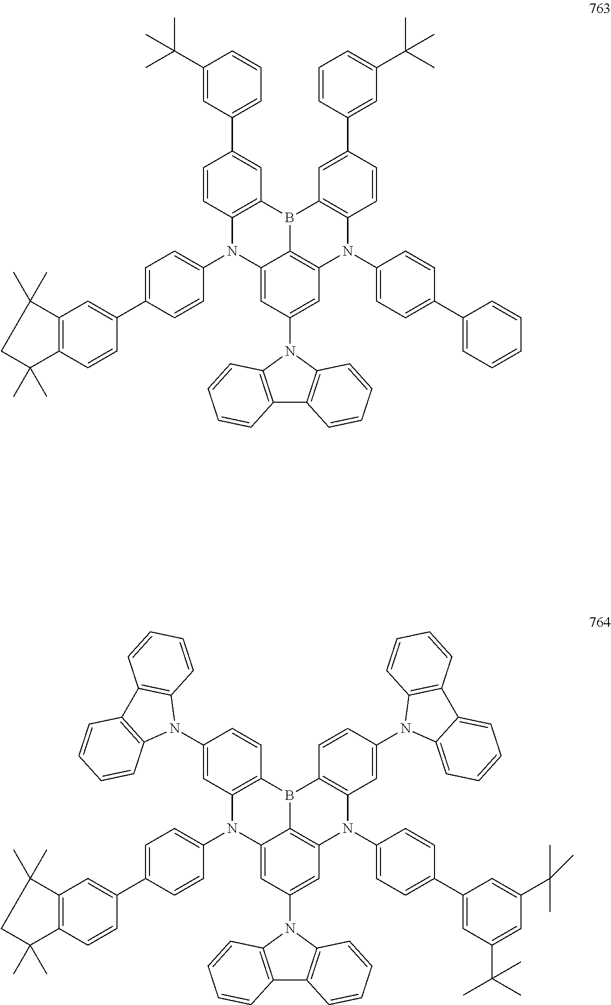 [SMILES: CC(C)(C)c1cccc(-c2ccc3c(c2)B2c4cc(-c5cccc(C(C)(C)C)c5)ccc4N(c4ccc(-c5ccc6c(c5)C(C)(C)CC6(C)C)cc4)c4cc(-n5c6ccccc6c6ccccc65)cc(c42)N3c2ccc(-c3ccccc3)cc2)c1 ; CC(C)(C)c1cc(-c2ccc(N3c4cc(-n5c6ccccc6c6ccccc65)ccc4B4c5ccc(-n6c7ccccc7c7ccccc76)cc5N(c5ccc(-c6ccc7c(c6)C(C)(C)CC7(C)C)cc5)c5cc(-n6c7ccccc7c7ccccc76)cc3c54)cc2)cc(C(C)(C)C)c1]

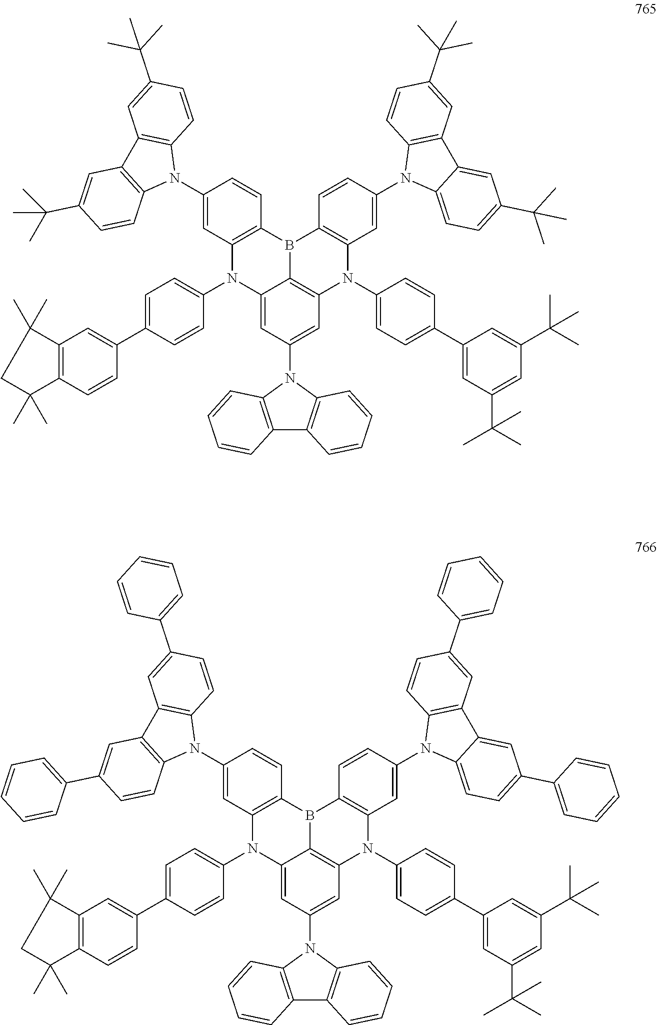 [SMILES: CC(C)(C)c1cc(-c2ccc(N3c4cc(-n5c6ccc(C(C)(C)C)cc6c6cc(C(C)(C)C)ccc65)ccc4B4c5ccc(-n6c7ccc(C(C)(C)C)cc7c7cc(C(C)(C)C)ccc76)cc5N(c5ccc(-c6ccc7c(c6)C(C)(C)CC7(C)C)cc5)c5cc(-n6c7ccccc7c7ccccc76)cc3c54)cc2)cc(C(C)(C)C)c1 ; CC(C)(C)c1cc(-c2ccc(N3c4cc(-n5c6ccc(-c7ccccc7)cc6c6cc(-c7ccccc7)ccc65)ccc4B4c5ccc(-n6c7ccc(-c8ccccc8)cc7c7cc(-c8ccccc8)ccc76)cc5N(c5ccc(-c6ccc7c(c6)C(C)(C)CC7(C)C)cc5)c5cc(-n6c7ccccc7c7ccccc76)cc3c54)cc2)cc(C(C)(C)C)c1]

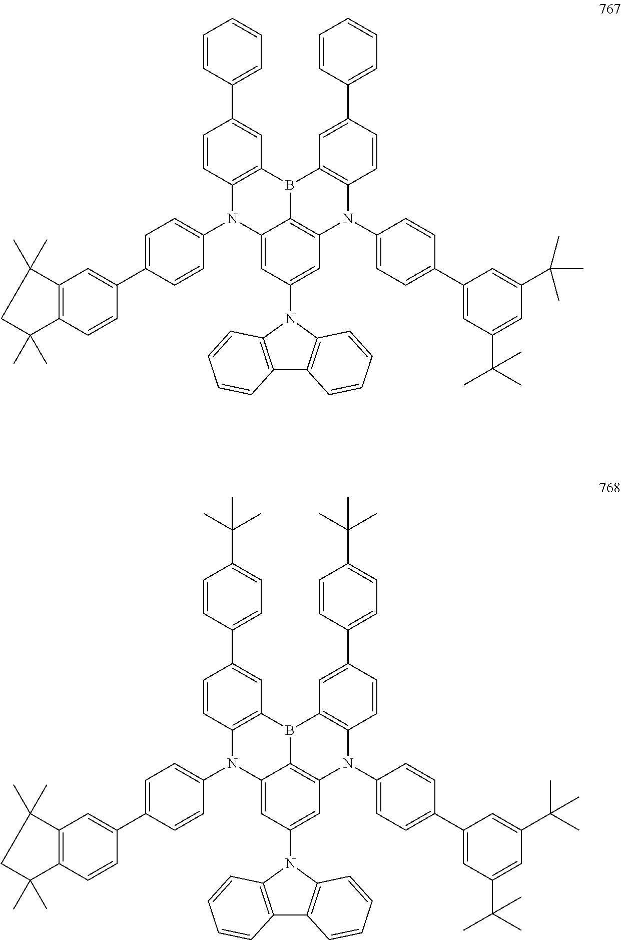 [SMILES: CC(C)(C)c1cc(-c2ccc(N3c4ccc(-c5ccccc5)cc4B4c5cc(-c6ccccc6)ccc5N(c5ccc(-c6ccc7c(c6)C(C)(C)CC7(C)C)cc5)c5cc(-n6c7ccccc7c7ccccc76)cc3c54)cc2)cc(C(C)(C)C)c1 ; CC(C)(C)c1ccc(-c2ccc3c(c2)B2c4cc(-c5ccc(C(C)(C)C)cc5)ccc4N(c4ccc(-c5ccc6c(c5)C(C)(C)CC6(C)C)cc4)c4cc(-n5c6ccccc6c6ccccc65)cc(c42)N3c2ccc(-c3cc(C(C)(C)C)cc(C(C)(C)C)c3)cc2)cc1]

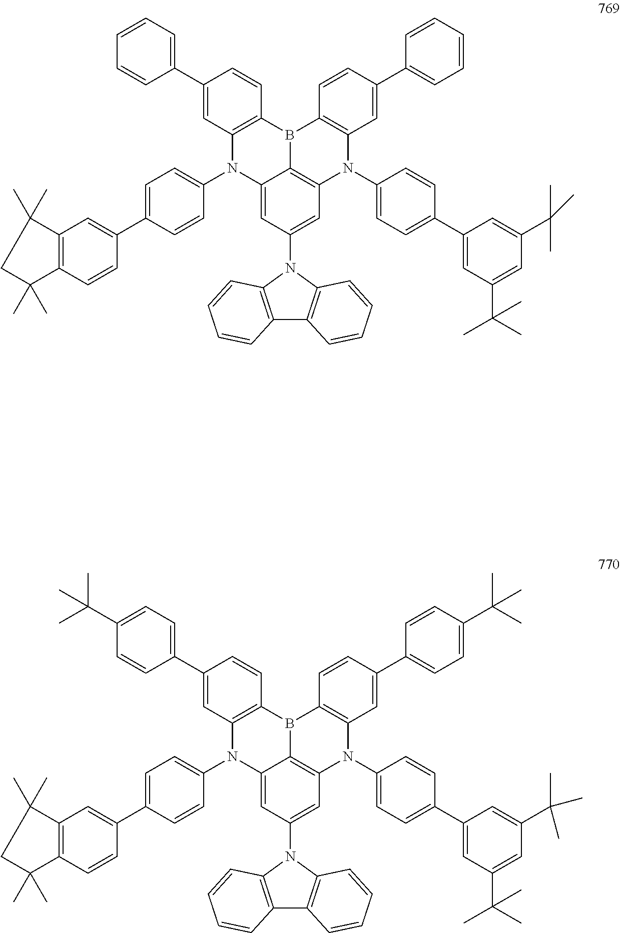 [SMILES: CC(C)(C)c1cc(-c2ccc(N3c4cc(-c5ccccc5)ccc4B4c5ccc(-c6ccccc6)cc5N(c5ccc(-c6ccc7c(c6)C(C)(C)CC7(C)C)cc5)c5cc(-n6c7ccccc7c7ccccc76)cc3c54)cc2)cc(C(C)(C)C)c1 ; CC(C)(C)c1ccc(-c2ccc3c(c2)N(c2ccc(-c4cc(C(C)(C)C)cc(C(C)(C)C)c4)cc2)c2cc(-n4c5ccccc5c5ccccc54)cc4c2B3c2ccc(-c3ccc(C(C)(C)C)cc3)cc2N4c2ccc(-c3ccc4c(c3)C(C)(C)CC4(C)C)cc2)cc1]

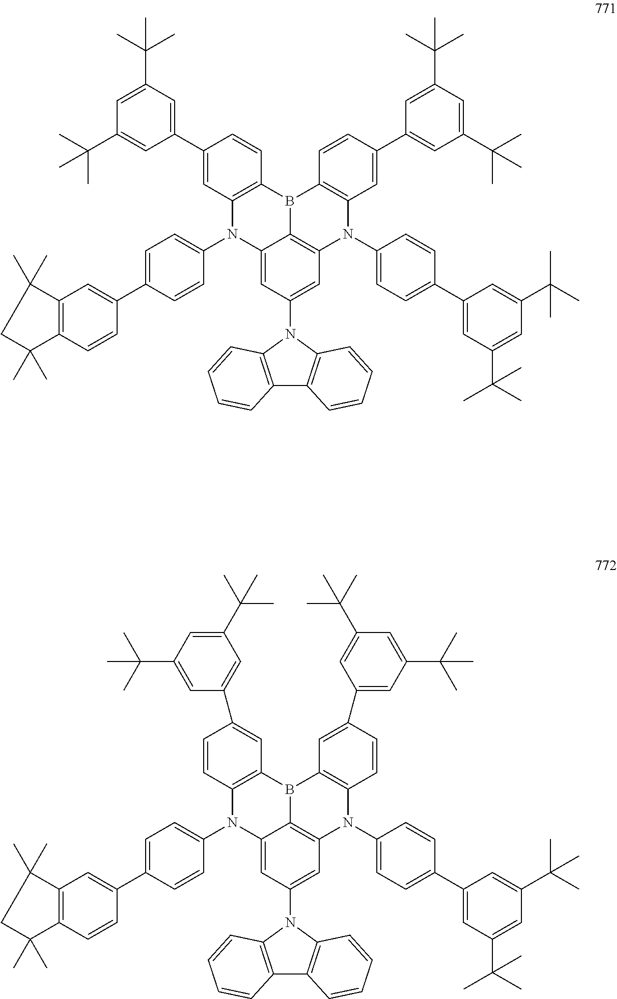 [SMILES: CC(C)(C)c1cc(-c2ccc(N3c4cc(-c5cc(C(C)(C)C)cc(C(C)(C)C)c5)ccc4B4c5ccc(-c6cc(C(C)(C)C)cc(C(C)(C)C)c6)cc5N(c5ccc(-c6ccc7c(c6)C(C)(C)CC7(C)C)cc5)c5cc(-n6c7ccccc7c7ccccc76)cc3c54)cc2)cc(C(C)(C)C)c1 ; CC(C)(C)c1cc(-c2ccc(N3c4ccc(-c5cc(C(C)(C)C)cc(C(C)(C)C)c5)cc4B4c5cc(-c6cc(C(C)(C)C)cc(C(C)(C)C)c6)ccc5N(c5ccc(-c6ccc7c(c6)C(C)(C)CC7(C)C)cc5)c5cc(-n6c7ccccc7c7ccccc76)cc3c54)cc2)cc(C(C)(C)C)c1]

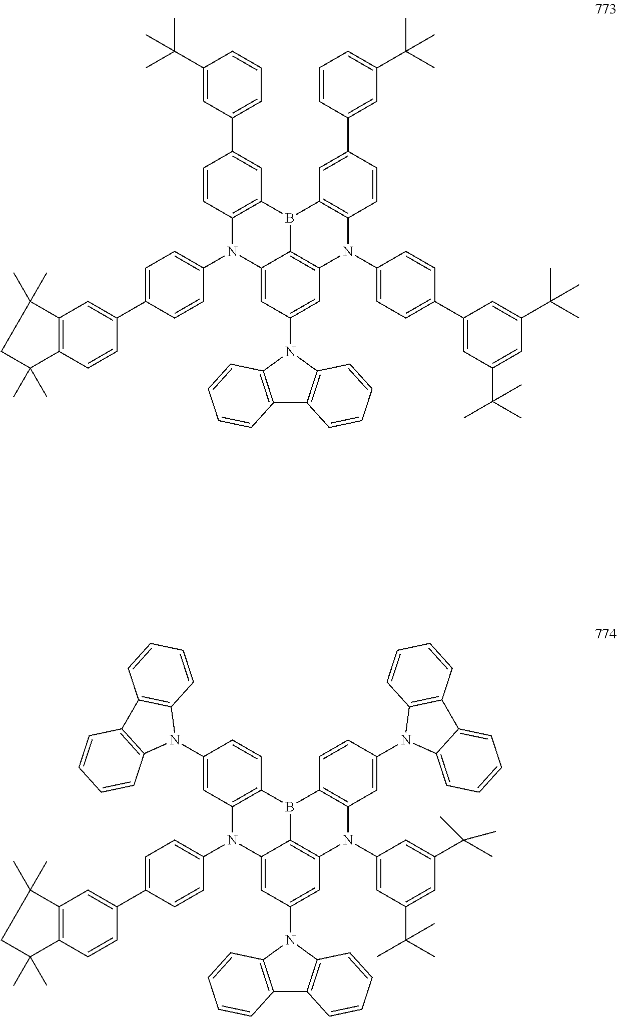 [SMILES: CC(C)(C)c1cccc(-c2ccc3c(c2)B2c4cc(-c5cccc(C(C)(C)C)c5)ccc4N(c4ccc(-c5ccc6c(c5)C(C)(C)CC6(C)C)cc4)c4cc(-n5c6ccccc6c6ccccc65)cc(c42)N3c2ccc(-c3cc(C(C)(C)C)cc(C(C)(C)C)c3)cc2)c1 ; CC(C)(C)c1cc(N2c3cc(-n4c5ccccc5c5ccccc54)ccc3B3c4ccc(-n5c6ccccc6c6ccccc65)cc4N(c4ccc(-c5ccc6c(c5)C(C)(C)CC6(C)C)cc4)c4cc(-n5c6ccccc6c6ccccc65)cc2c43)cc(C(C)(C)C)c1]

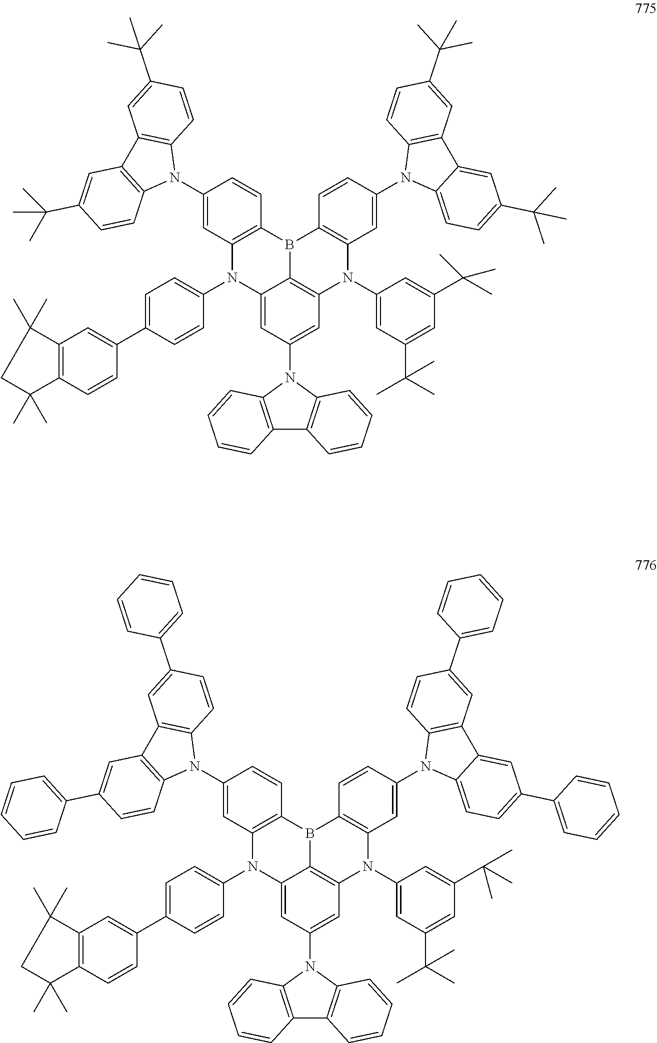 [SMILES: CC(C)(C)c1cc(N2c3cc(-n4c5ccc(C(C)(C)C)cc5c5cc(C(C)(C)C)ccc54)ccc3B3c4ccc(-n5c6ccc(C(C)(C)C)cc6c6cc(C(C)(C)C)ccc65)cc4N(c4ccc(-c5ccc6c(c5)C(C)(C)CC6(C)C)cc4)c4cc(-n5c6ccccc6c6ccccc65)cc2c43)cc(C(C)(C)C)c1 ; CC(C)(C)c1cc(N2c3cc(-n4c5ccc(-c6ccccc6)cc5c5cc(-c6ccccc6)ccc54)ccc3B3c4ccc(-n5c6ccc(-c7ccccc7)cc6c6cc(-c7ccccc7)ccc65)cc4N(c4ccc(-c5ccc6c(c5)C(C)(C)CC6(C)C)cc4)c4cc(-n5c6ccccc6c6ccccc65)cc2c43)cc(C(C)(C)C)c1]

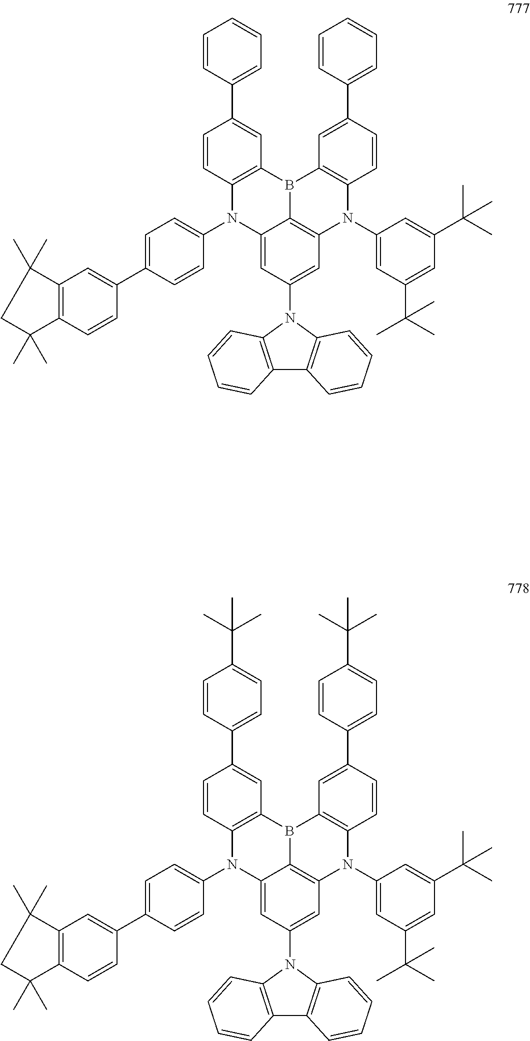 [SMILES: CC(C)(C)c1cc(N2c3ccc(-c4ccccc4)cc3B3c4cc(-c5ccccc5)ccc4N(c4ccc(-c5ccc6c(c5)C(C)(C)CC6(C)C)cc4)c4cc(-n5c6ccccc6c6ccccc65)cc2c43)cc(C(C)(C)C)c1 ; CC(C)(C)c1ccc(-c2ccc3c(c2)B2c4cc(-c5ccc(C(C)(C)C)cc5)ccc4N(c4cc(C(C)(C)C)cc(C(C)(C)C)c4)c4cc(-n5c6ccccc6c6ccccc65)cc(c42)N3c2ccc(-c3ccc4c(c3)C(C)(C)CC4(C)C)cc2)cc1]

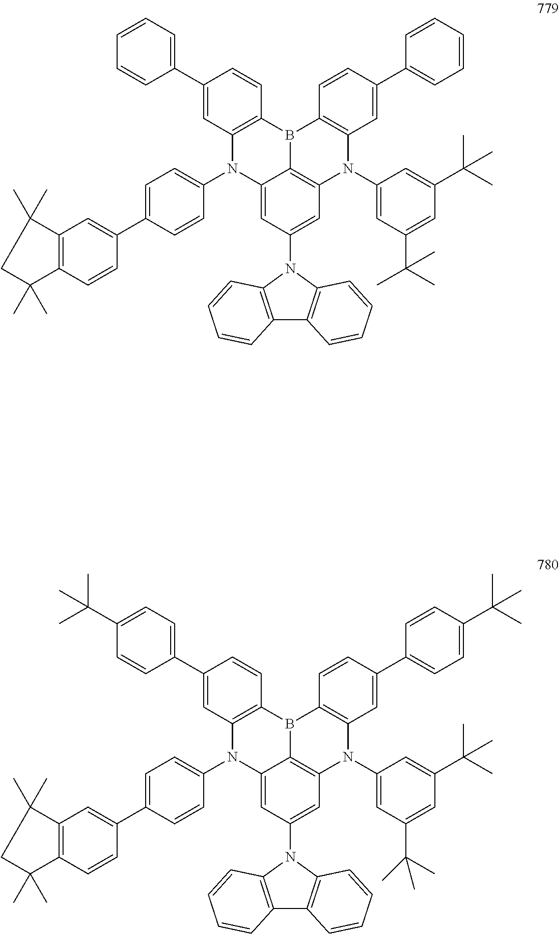 [SMILES: CC(C)(C)c1cc(N2c3cc(-c4ccccc4)ccc3B3c4ccc(-c5ccccc5)cc4N(c4ccc(-c5ccc6c(c5)C(C)(C)CC6(C)C)cc4)c4cc(-n5c6ccccc6c6ccccc65)cc2c43)cc(C(C)(C)C)c1 ; CC(C)(C)c1ccc(-c2ccc3c(c2)N(c2ccc(-c4ccc5c(c4)C(C)(C)CC5(C)C)cc2)c2cc(-n4c5ccccc5c5ccccc54)cc4c2B3c2ccc(-c3ccc(C(C)(C)C)cc3)cc2N4c2cc(C(C)(C)C)cc(C(C)(C)C)c2)cc1]

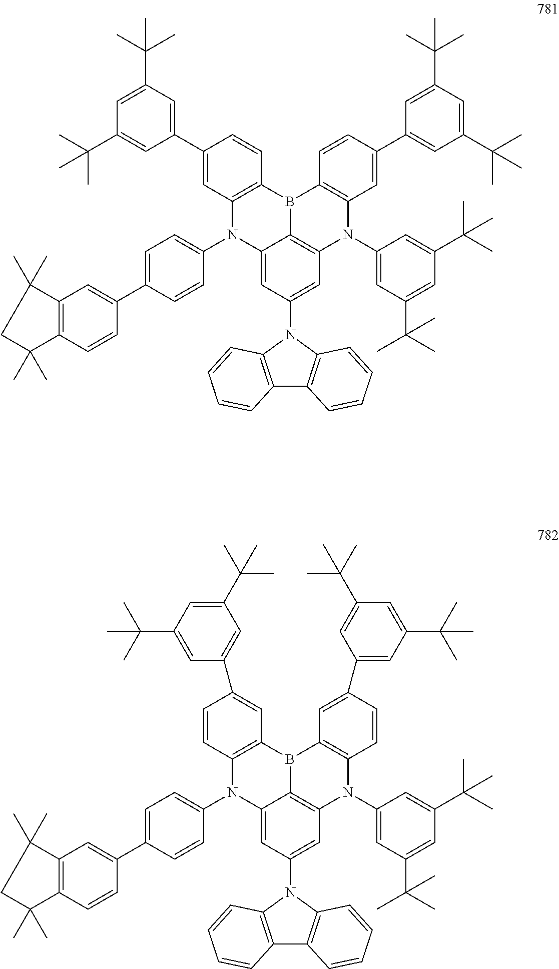 [SMILES: CC(C)(C)c1cc(-c2ccc3c(c2)N(c2ccc(-c4ccc5c(c4)C(C)(C)CC5(C)C)cc2)c2cc(-n4c5ccccc5c5ccccc54)cc4c2B3c2ccc(-c3cc(C(C)(C)C)cc(C(C)(C)C)c3)cc2N4c2cc(C(C)(C)C)cc(C(C)(C)C)c2)cc(C(C)(C)C)c1 ; CC(C)(C)c1cc(-c2ccc3c(c2)B2c4cc(-c5cc(C(C)(C)C)cc(C(C)(C)C)c5)ccc4N(c4cc(C(C)(C)C)cc(C(C)(C)C)c4)c4cc(-n5c6ccccc6c6ccccc65)cc(c42)N3c2ccc(-c3ccc4c(c3)C(C)(C)CC4(C)C)cc2)cc(C(C)(C)C)c1]

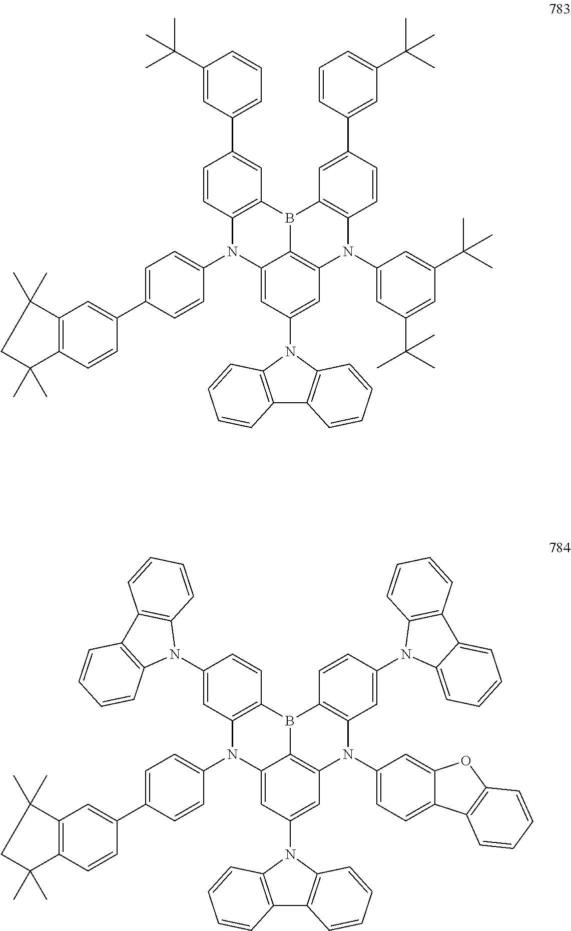 [SMILES: CC(C)(C)c1cccc(-c2ccc3c(c2)B2c4cc(-c5cccc(C(C)(C)C)c5)ccc4N(c4cc(C(C)(C)C)cc(C(C)(C)C)c4)c4cc(-n5c6ccccc6c6ccccc65)cc(c42)N3c2ccc(-c3ccc4c(c3)C(C)(C)CC4(C)C)cc2)c1 ; CC1(C)CC(C)(C)c2cc(-c3ccc(N4c5cc(-n6c7ccccc7c7ccccc76)ccc5B5c6ccc(-n7c8ccccc8c8ccccc87)cc6N(c6ccc7c(c6)oc6ccccc67)c6cc(-n7c8ccccc8c8ccccc87)cc4c65)cc3)ccc21]

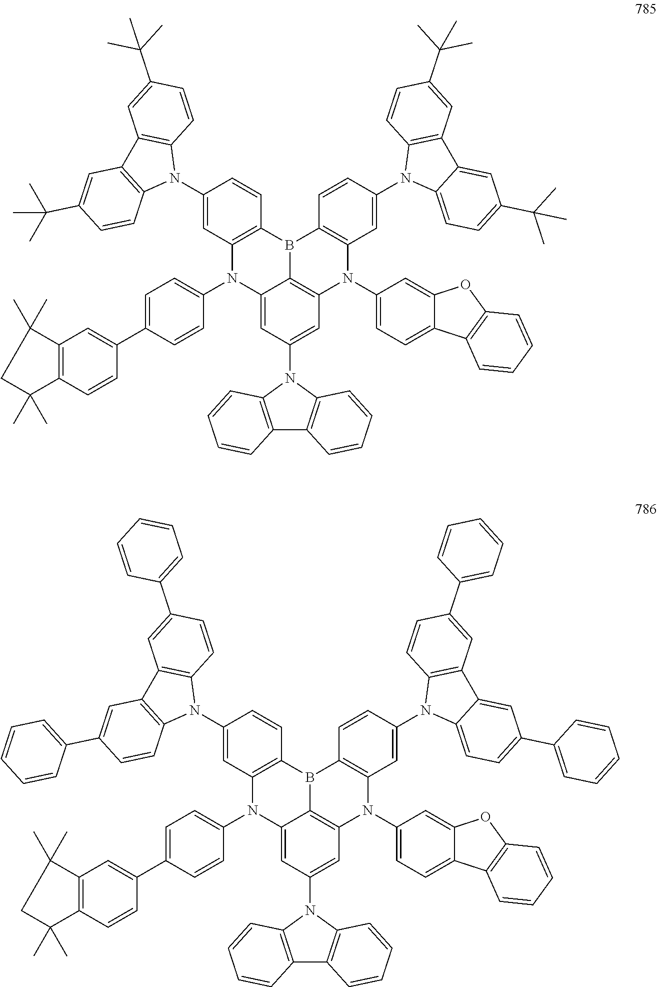 [SMILES: CC(C)(C)c1ccc2c(c1)c1cc(C(C)(C)C)ccc1n2-c1ccc2c(c1)N(c1ccc(-c3ccc4c(c3)C(C)(C)CC4(C)C)cc1)c1cc(-n3c4ccccc4c4ccccc43)cc3c1B2c1ccc(-n2c4ccc(C(C)(C)C)cc4c4cc(C(C)(C)C)ccc42)cc1N3c1ccc2c(c1)oc1ccccc12 ; CC1(C)CC(C)(C)c2cc(-c3ccc(N4c5cc(-n6c7ccc(-c8ccccc8)cc7c7cc(-c8ccccc8)ccc76)ccc5B5c6ccc(-n7c8ccc(-c9ccccc9)cc8c8cc(-c9ccccc9)ccc87)cc6N(c6ccc7c(c6)oc6ccccc67)c6cc(-n7c8ccccc8c8ccccc87)cc4c65)cc3)ccc21]

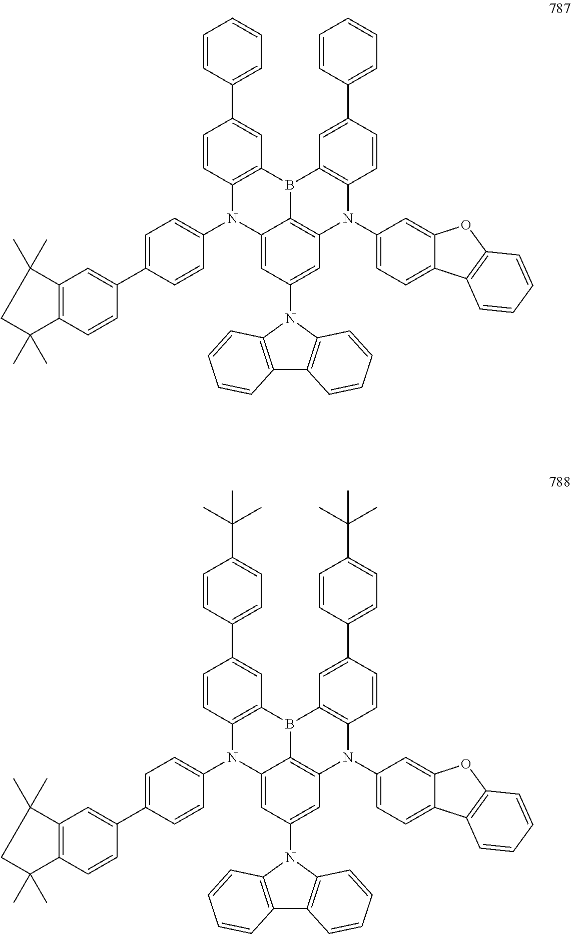 [SMILES: CC1(C)CC(C)(C)c2cc(-c3ccc(N4c5ccc(-c6ccccc6)cc5B5c6cc(-c7ccccc7)ccc6N(c6ccc7c(c6)oc6ccccc67)c6cc(-n7c8ccccc8c8ccccc87)cc4c65)cc3)ccc21 ; CC(C)(C)c1ccc(-c2ccc3c(c2)B2c4cc(-c5ccc(C(C)(C)C)cc5)ccc4N(c4ccc5c(c4)oc4ccccc45)c4cc(-n5c6ccccc6c6ccccc65)cc(c42)N3c2ccc(-c3ccc4c(c3)C(C)(C)CC4(C)C)cc2)cc1]

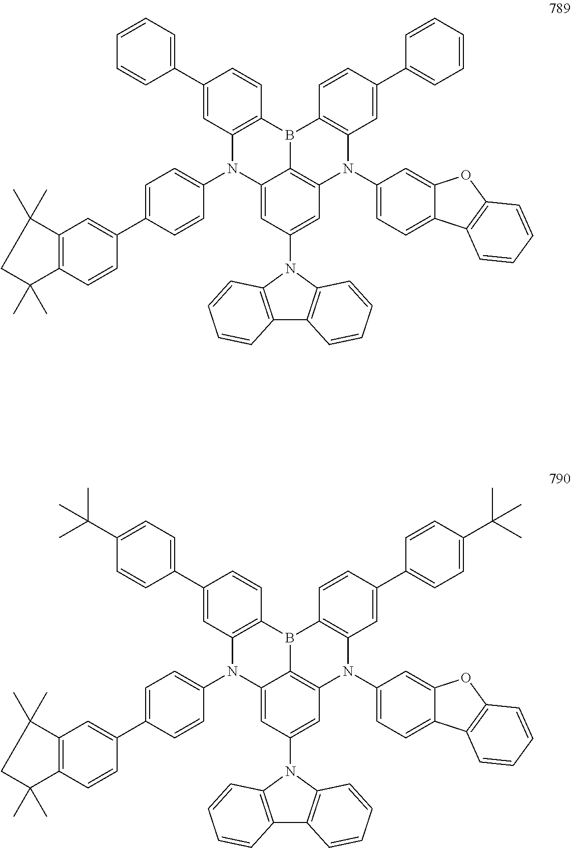 [SMILES: CC1(C)CC(C)(C)c2cc(-c3ccc(N4c5cc(-c6ccccc6)ccc5B5c6ccc(-c7ccccc7)cc6N(c6ccc7c(c6)oc6ccccc67)c6cc(-n7c8ccccc8c8ccccc87)cc4c65)cc3)ccc21 ; CC(C)(C)c1ccc(-c2ccc3c(c2)N(c2ccc(-c4ccc5c(c4)C(C)(C)CC5(C)C)cc2)c2cc(-n4c5ccccc5c5ccccc54)cc4c2B3c2ccc(-c3ccc(C(C)(C)C)cc3)cc2N4c2ccc3c(c2)oc2ccccc23)cc1]

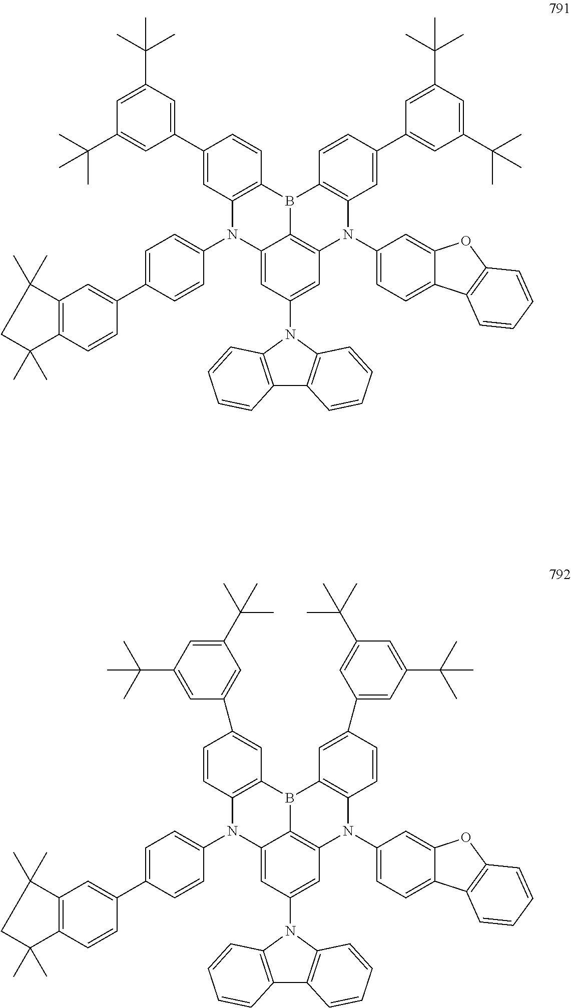 [SMILES: CC(C)(C)c1cc(-c2ccc3c(c2)N(c2ccc(-c4ccc5c(c4)C(C)(C)CC5(C)C)cc2)c2cc(-n4c5ccccc5c5ccccc54)cc4c2B3c2ccc(-c3cc(C(C)(C)C)cc(C(C)(C)C)c3)cc2N4c2ccc3c(c2)oc2ccccc23)cc(C(C)(C)C)c1 ; CC(C)(C)c1cc(-c2ccc3c(c2)B2c4cc(-c5cc(C(C)(C)C)cc(C(C)(C)C)c5)ccc4N(c4ccc5c(c4)oc4ccccc45)c4cc(-n5c6ccccc6c6ccccc65)cc(c42)N3c2ccc(-c3ccc4c(c3)C(C)(C)CC4(C)C)cc2)cc(C(C)(C)C)c1]

The heterocyclic compound represented by Formula 1 satisfies the structure of Formula 1 described above, and includes a structure in which a group represented by Formula 2 is substituted in ring CY₄ of Formula 1. Due to this structure, the heterocyclic compound represented by Formula 1 has excellent luminescent characteristics, and in particular, may implement deep blue light emission having a short wavelength.

Although not limited by a particular theory, the heterocyclic compound that satisfies the structure as described above may have high oscillator strength (f), and thus, when the heterocyclic compound is applied to an electronic device, efficiency of the electronic device may increase. In addition, since the heterocyclic compound includes a substituent having a bulky structure represented by Formula 2 as described above, the heterocyclic compound may have a high S₁ energy level and a high photoalignment. Accordingly, when the heterocyclic compound is used as a luminescent material, Dexter energy transfer is suppressed and excellent lifespan characteristics may be exhibited.

Therefore, an electronic device, for example, an organic light-emitting device, including the heterocyclic compound represented by Formula 1 may have high efficiency and long lifespan.

The highest occupied molecular orbital (HOMO) energy level, lowest unoccupied molecular orbital (LUMO) energy level, S₁ energy level, T₁ energy level, and oscillator strength (f) of some of the heterocyclic compounds represented by Formula 1 were evaluated using the Gaussian 09 program with the molecular structure optimization obtained by B3LYP-based density functional theory (DFT), and results thereof are shown in Table 1.

TABLE 1

| | HOMO (eV) | LUMO (eV) | S₁ (eV) | T₁ (eV) | f |
|---|---|---|---|---|---|
| Compound | −4.873 | −1.351 | 2.684 | 2.541 | 0.588 |
| 1 | | | | | |
| Compound | −4.754 | −1.092 | 2.737 | 2.567 | 0.309 |
| A | | | | | |
| Compound | −4.642 | −0.997 | 2.678 | 2.546 | 0.334 |
| B | | | | | |
| Compound | −4.70 | −1.12 | 2.74 | 2.61 | 0.355 |
| C | | | | | |
| Compound | −4.66 | −1.05 | 2.76 | 2.64 | 0.335 |
| D | | | | | |

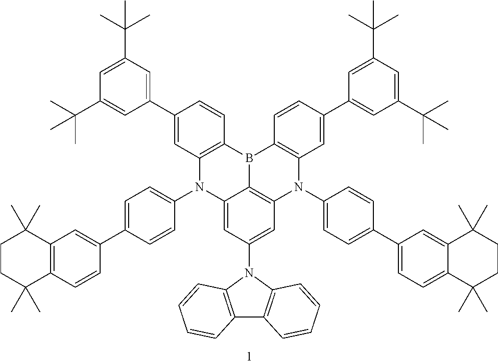 [SMILES: CC(C)(C)c1cc(-c2ccc3c(c2)N(c2ccc(-c4ccc5c(c4)C(C)(C)CCC5(C)C)cc2)c2cc(-n4c5ccccc5c5ccccc54)cc4c2B3c2ccc(-c3cc(C(C)(C)C)cc(C(C)(C)C)c3)cc2N4c2ccc(-c3ccc4c(c3)C(C)(C)CCC4(C)C)cc2)cc(C(C)(C)C)c1]

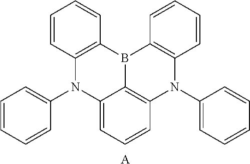

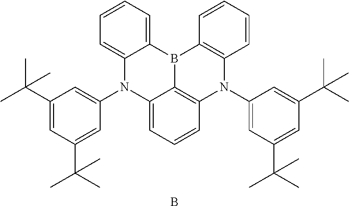

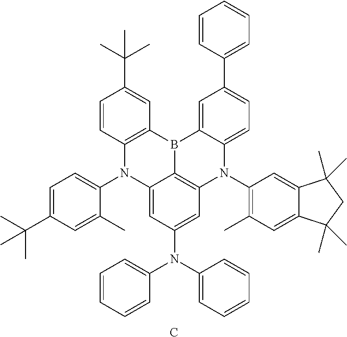

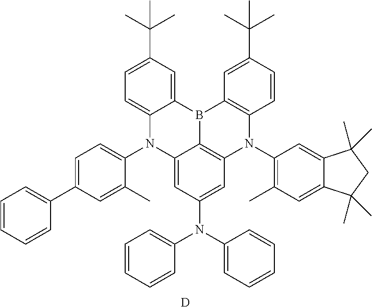

From the Table 1, it may be seen that the heterocyclic compound represented by Formula 1 has electric characteristics suitable for use as a dopant (for example, an emitter or a sensitizer) in an electronic device, for example, an organic light-emitting device. For example, the heterocyclic compound has a deeper HOMO value and a significantly higher f value, as compared to Compounds A to D as comparative compounds.

In an embodiment, a full width at half maximum (FWHM) of an emission peak of an emission spectrum or an electroluminescence (EL) spectrum of the heterocyclic compound may be 60 nm or less. For example, an FWHM of an emission peak of an emission spectrum or an EL spectrum of the heterocyclic compound may be 5 nm to 50 nm, 7 nm to 40 nm, or 10 nm to 30 nm.

Synthesis method of the heterocyclic compound represented by Formula 1 may be recognized by those skilled in the art with reference to the following Synthesis Examples.

A method of identifying a structure of the heterocyclic compound represented by Formula 1 is not particularly limited. In an embodiment, the structure of the heterocyclic compound may be identified by a known method (for example, nuclear magnetic resonance (NMR) or liquid chromatography-mass spectrometry (LC-MS)).

### [Organic Light-Emitting Device]

According to one or more embodiments, an organic light-emitting device includes the heterocyclic compound.

In an embodiment, the organic light-emitting device may include: a first electrode; a second electrode; and an organic layer arranged between the first electrode and the second electrode and including an emission layer, and the organic layer may include the heterocyclic compound represented by Formula 1.

In an embodiment, the emission layer may include the heterocyclic compound.

In an embodiment, the emission layer may include a host and an emitter, and the emitter may include the heterocyclic compound.

In an embodiment, in the emission layer, an amount of the host may be greater than an amount of the heterocyclic compound based on the weight.

In an embodiment, the emission layer may further include a sensitizer.

In an embodiment, the sensitizer may include a phosphorescent compound, a delayed fluorescence compound, or any combination thereof.

The host, the emitter, and the sensitizer are each the same as described in the present specification.

Since the organic light-emitting device includes the emission layer including the heterocyclic compound represented by Formula 1 as described above, the organic light-emitting device may have a relatively narrow FWHM of an emission peak of an EL spectrum, excellent efficiency, and excellent lifespan characteristics.

In an embodiment, the heterocyclic compound may act as a dopant (for example, an emitter or a sensitizer) in the emission layer, and the emission layer may further include a host (in other words, in the emission layer, an amount of the heterocyclic compound represented by Formula 1 may be less than an amount of the host).

In an embodiment, the emission layer may emit blue light. For example, the emission layer may emit blue light having a maximum emission wavelength of about 410 nm to about 490 nm.

The wording “(emission layer) includes at least one heterocyclic compound” as used herein may be to mean that the (emission layer) may include one kind of heterocyclic compound represented by Formula 1 or two or more different kinds of heterocyclic compounds, each represented by Formula 1.

For example, the emission layer may include, as the heterocyclic compound, only Compound 1. In this regard, Compound 1 may be included in the emission layer of the organic light-emitting device. In an embodiment, the emission layer may include, as the heterocyclic compound, Compound 1 and Compound 2.

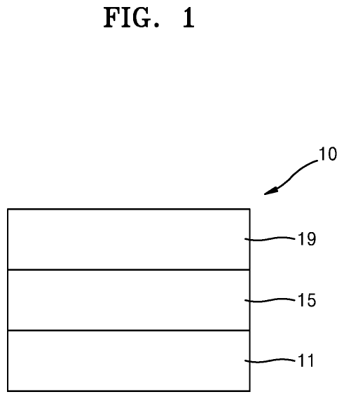

### Description of FIG. 1

FIG. 1 is a schematic cross-sectional view of an organic light-emitting device 10 according to an embodiment. Hereinafter, referring to FIG. 1, a structure and manufacturing method of an organic light-emitting device according to an embodiment are described as follows.

In FIG. 1, the organic light-emitting device 10 includes a first electrode 11, a second electrode 19 facing the first electrode 11, and an organic layer 15 between the first electrode 11 and the second electrode 19.

The organic layer 15 includes an emission layer, a hole transport region may be arranged between the first electrode 11 and the emission layer, and an electron transport region may be arranged between the emission layer and the second electrode 19.

A substrate may be additionally disposed under the first electrode 11 or on the second electrode 19. The substrate may be a conventional substrate used in organic light-emitting devices, e.g., a glass substrate or a transparent plastic substrate, each having excellent mechanical strength, thermal stability, transparency, surface smoothness, ease of handling, and water repellency.

### [First Electrode 11]

The first electrode 11 may be produced by depositing or sputtering, onto the substrate, a material for forming the first electrode 11. The first electrode 11 may be an anode. The material for forming the first electrode 11 may be selected from materials with a high work function for easy hole injection.

The first electrode 11 may be a reflective electrode, a semi-transmissive electrode, or a transmissive electrode. When the first electrode 11 is a transmissive electrode, a material for forming the first electrode 11 may be indium tin oxide (ITO), indium zinc oxide (IZO), tin oxide (SnO₂), zinc oxide (ZnO), or any combination thereof, but embodiments are not limited thereto. In some embodiments, when the first electrode 11 is a semi-transmissive electrode or a reflective electrode, as a material for forming the first electrode 11, at least one of magnesium (Mg), silver (Ag), aluminum (Al), aluminum-lithium (Al—Li), calcium (Ca), magnesium-indium (Mg—In), magnesium-silver (Mg—Ag), or any combination thereof may be used, but embodiments are not limited thereto.

The first electrode 11 may have a single-layered structure or a multilayer structure including a plurality of layers.

### [Emission Layer]

The emission layer may include the heterocyclic compound.

A thickness of the emission layer may be in a range of about 100 Å to about 1,000 Å, for example, about 200 Å to about 600 Å. When the thickness of the emission layer is within the range, excellent light-emission characteristics may be obtained without a substantial increase in driving voltage.

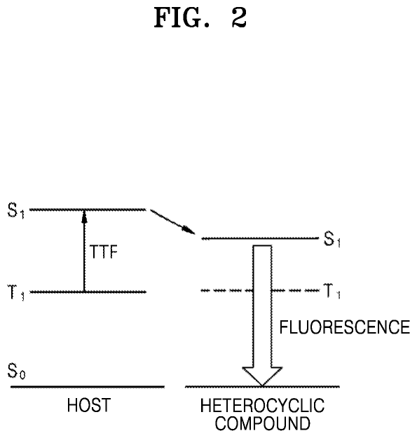

### Description of FIG. 2

In an embodiment, the heterocyclic compound may be a fluorescent emitter.

In an embodiment, the emission layer may further include a host (hereinafter, referred to as ‘Host A’, and Host A may not be identical to the heterocyclic compound). Host A may be understood by referring to the description of a host material described below, but embodiments are not limited thereto. Host A may be a fluorescent host.

Referring to FIG. 2, energy transfer according to an embodiment is described as follows.

Singlet excitons are formed in Host A in the emission layer, and the singlet excitons formed in Host A are transferred to a fluorescent emitter through Förster energy transfer (or, Förster resonance energy transfer (FRET)).

A proportion of the singlet excitons formed in Host A may be only 25%, and thus, 75% of triplet excitons formed in Host A may be fused to each other to be converted into singlet excitons. Thus, efficiency of the organic light-emitting device may be further improved. In other words, the efficiency of the organic light-emitting device may be further improved using a triplet-triplet fusion (TTF) mechanism.

In an embodiment, among total emission components emitted from the emission layer, a proportion of emission components emitted from the heterocyclic compound may be greater than or equal to 80%, for example, greater than or equal to 90%. For example, among total emission components emitted from the emission layer, a proportion of emission components emitted from the heterocyclic compound may be greater than or equal to 95%.

The heterocyclic compound may emit fluorescence, and the host may not emit light.

In an embodiment, when the emission layer further includes Host A, in addition to the heterocyclic compound, an amount of the heterocyclic compound may be about 50 parts by weight or less, for example, about 30 parts by weight or less, about 10 parts by weight or less, or about 1 part by weight to about 5 parts by weight, based on 100 parts by weight of the emission layer, and an amount of Host A in the emission layer may be about 50 parts by weight or greater, for example, about 70 parts by weight or greater, about 90 parts by weight or greater, or about 95 part by weight to about 99 parts by weight, based on 100 parts by weight of the emission layer, but embodiments are not limited thereto.

In an embodiment, when the emission layer further includes Host A, in addition to the heterocyclic compound, Host A and the heterocyclic compound may satisfy Condition A:

E(H_{A})_{S1}>E_{S1} Condition A
wherein, in Condition A,
E(H_{A})_{S1} indicates a lowest excited singlet energy level of Host A, and
E_{S1} indicates a lowest excited singlet energy level of the heterocyclic compound.

Here, E(H_{A})_{S1} and E_{S1} are evaluated using the DFT method of the Gaussian program, wherein structure optimization is performed at B3LYP/6-31G(d,p) level.

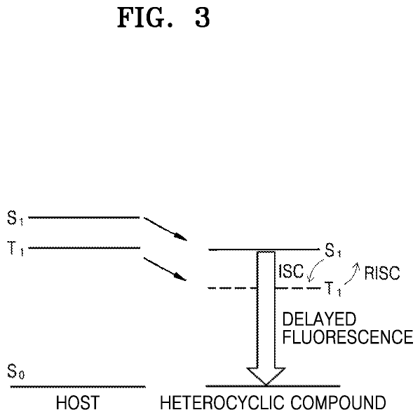

### Description for FIG. 3

In an embodiment, the heterocyclic compound may be a delayed fluorescence emitter.

In an embodiment, the emission layer may further include a host (hereinafter, referred to as ‘Host B’, and Host B is not identical to the heterocyclic compound). Host B may be understood by referring to the description of a host material described below, but embodiments are not limited thereto.

Referring to FIG. 3, energy transfer according to an embodiment is described as follows.

25% of singlet excitons formed in Host B in the emission layer are transferred to a delayed fluorescence emitter through FRET. In addition, 75% of triplet excitons formed in Host B in the emission layer are transferred to a delayed fluorescence emitter through Dexter energy transfer. At least a portion of energy of a singlet state of the delayed fluorescence emitter may be transferred to a triplet state by intersystem crossing (ISC). The energy transferred to the triplet state of the delayed fluorescence emitter may be transferred to the singlet state by reverse intersystem crossing (RISC). Accordingly, by transferring all the singlet excitons and triplet excitons generated in the emission layer to the heterocyclic compound, the organic light-emitting device having improved efficiency may be obtained.

Therefore, in an embodiment, among total emission components emitted from the emission layer, a proportion of emission components emitted from the heterocyclic compound may be greater than or equal to 80%, for example, greater than or equal to 90%. For example, among total emission components emitted from the emission layer, a proportion of emission components emitted from the heterocyclic compound may be greater than or equal to 95%.

Here, the heterocyclic compound may emit fluorescence and/or delayed fluorescence, and the emission components of the heterocyclic compound may be a total of prompt emission components of the heterocyclic compound and delayed fluorescence components by RISC of the heterocyclic compound. In addition, Host B may not emit light.

In an embodiment, when the emission layer further includes Host B, in addition to the heterocyclic compound, an amount of the heterocyclic compound may be about 50 parts by weight or less, for example, about 30 parts by weight or less, based on 100 parts by weight of the emission layer, and an amount of Host B in the emission layer may be about 50 parts by weight or greater, for example, about 70 parts by weight or greater, based on 100 parts by weight of the emission layer, but embodiments are not limited thereto.

In an embodiment, when the emission layer further includes Host B, in addition to the heterocyclic compound, Host B and the heterocyclic compound may satisfy Condition B:

E(H_{B})_{S1}>E_{S1} Condition B
wherein, in Condition B,
E(H_{B})_{S1} indicates a lowest excited singlet energy level of Host B, and
E_{S1} indicates a lowest excited singlet energy level of the heterocyclic compound.

Here, E(H_{B})_{S1} and E_{S1} are evaluated using the DFT method of the Gaussian program, wherein structure optimization is performed at B3LYP/6-31G(d,p) level.

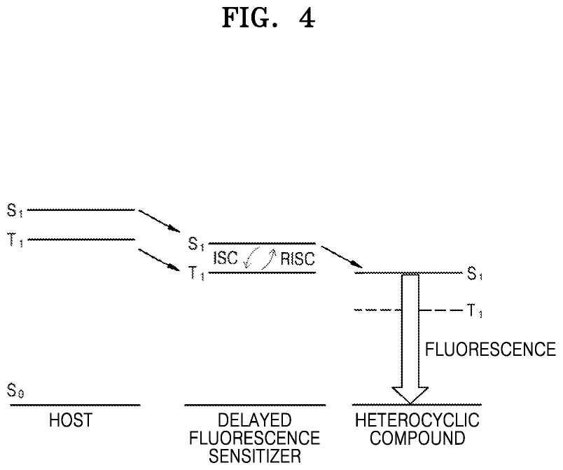

### Description of FIG. 4

In an embodiment, the heterocyclic compound may be used as a fluorescent emitter, and the emission layer may further include a sensitizer, for example, a delayed fluorescence sensitizer. In an embodiment, the emission layer may further include a host (hereinafter, referred to as ‘Host C’, and Host C is not identical to the heterocyclic compound and the sensitizer) and a sensitizer (hereinafter, referred to as ‘Sensitizer A’, and Sensitizer A is not identical to Host C and the heterocyclic compound). Host C and Sensitizer A may respectively be understood by referring to the descriptions of a host material and a sensitizer material described below, but embodiments are not limited thereto.

In an embodiment, among total emission components emitted from the emission layer, a proportion of emission components of the heterocyclic compound may be greater than or equal to 80%, for example, greater than or equal to 90% (or for example, greater than or equal to 95%). For example, the heterocyclic compound may emit fluorescence. In addition, Host C and Sensitizer A may not each emit light.

Referring to FIG. 4, energy transfer according to an embodiment is described as follows.

Singlet and triplet excitons are formed in Host C in the emission layer, and the singlet and triplet excitons formed in Host C may be transferred to Sensitizer A and then to the heterocyclic compound through FRET. 25% of the singlet excitons are formed in Host C and are transferred to Sensitizer A through FRET, and 75% of the triplet excitons are formed in Host C and energy thereof is transferred to a singlet state and triplet state of Sensitizer A. At least a portion of energy of the singlet state of Sensitizer A may be transferred to the triplet state by ISC. The energy transferred to the triplet state of Sensitizer A is transferred to the singlet state by RISC, and then, the singlet state energy of Sensitizer A is transferred to the heterocyclic compound through FRET.

Accordingly, by transferring all the singlet excitons and triplet excitons generated in the emission layer to a dopant (for example, an emitter), the organic light-emitting device having improved efficiency may be obtained. In addition, since the organic light-emitting device with significantly reduced energy loss may be obtained, lifespan characteristics of the organic light-emitting device may be improved.

Referring to FIG. 4, when the emission layer further includes Host C and Sensitizer A, in addition to the heterocyclic compound, Host C, Sensitizer A and the heterocyclic compound may satisfy Condition C-1 and/or C-2:

S₁(H_{C})≥S₁(S_{A}) Condition C-1

S₁(S_{A})≥S₁(HC) Condition C-2
wherein, in Conditions C-1 and C-2,
S₁(H_{C}) indicates a lowest excited singlet energy level of Host C,
S₁(S_{A}) indicates a lowest excited singlet energy level of Sensitizer A, and
S₁(HC) indicates a lowest excited singlet energy level of the heterocyclic compound.

S₁(H_{C}), S₁(S_{A}), and S₁(HC) are evaluated using the DFT method of the Gaussian program, wherein structure optimization is performed at B3LYP/6-31G(d,p) level.

When Host C, Sensitizer A, and the heterocyclic compound satisfy Condition C-1 and/or C-2, FRET from Sensitizer A to the heterocyclic compound may be facilitated, and accordingly, the organic light-emitting device may have improved emission efficiency.

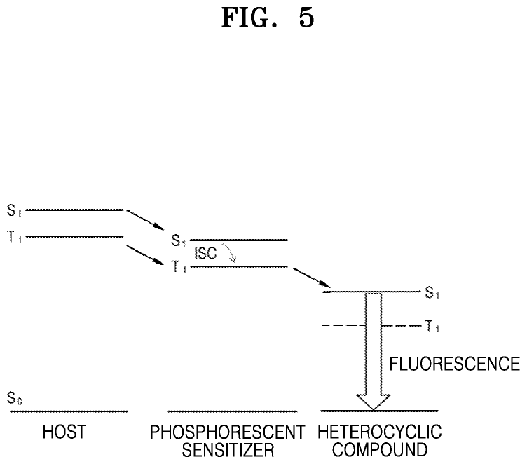

### Description of FIG. 5

In an embodiment, the heterocyclic compound may be used as a fluorescent emitter, and the emission layer may further include a sensitizer, for example, a phosphorescent sensitizer.

In an embodiment, the emission layer may further include a host (hereinafter, referred to as ‘Host D’, and Host D is not identical to the heterocyclic compound and the sensitizer) and a sensitizer (hereinafter, referred to as ‘Sensitizer B’, and Sensitizer B is not identical to Host D and the heterocyclic compound). Host D and Sensitizer B may respectively be understood by referring to the descriptions of a host material and a sensitizer material described below, but embodiments are not limited thereto.

In an embodiment, among total emission components emitted from the emission layer, a proportion of emission components of the heterocyclic compound may be greater than or equal to 80%, for example, greater than or equal to 90% (or for example, greater than or equal to 95%). For example, the heterocyclic compound may emit fluorescence. In addition, Host D and Sensitizer B may not each emit light.

Referring to FIG. 5, energy transfer according to an embodiment is described as follows.

75% of triplet excitons are formed in Host D in the emission layer and are transferred to Sensitizer B through Dexter energy transfer, and 25% of singlet excitons are formed in Host D and energy thereof is transferred to a singlet state and triplet state of Sensitizer B. The energy transferred to the singlet state of Sensitizer B is transferred to the triplet state by ISC, and then, the triplet energy of Sensitizer B is transferred to the heterocyclic compound through FRET.

Accordingly, by transferring all the singlet excitons and triplet excitons generated in the emission layer to a dopant (for example, an emitter), the organic light-emitting device having improved efficiency may be obtained. In addition, since the organic light-emitting device with significantly reduced energy loss may be obtained, lifespan characteristics of the organic light-emitting device may be improved.

In an embodiment, when the emission layer further includes Host D and Sensitizer B, in addition to the heterocyclic compound, Host D, Sensitizer B, and the heterocyclic compound may satisfy Condition D-1 and/or D-2:

T₁(H_{D})≥T₁(S_{B}) Condition D-1

T₁(S_{B})≥S₁(HC) Condition D-2
wherein, in Conditions D-1 and D-2,
T₁(H_{D}) indicates a lowest excited triplet energy level of Host D,
T₁(S_{B}) indicates a lowest excited triplet energy level of Sensitizer B, and
S₁(HC) indicates a lowest excited singlet energy level of the heterocyclic compound.

T₁(H_{D}), T₁(S_{B}), and S₁(HC) are evaluated using the DFT method of the Gaussian program, wherein structure optimization is performed at B3LYP/6-31G(d,p) level.

When Host D, Sensitizer B, and the heterocyclic compound satisfy Condition D-1 and/or D-2, FRET from Sensitizer B to the heterocyclic compound may be facilitated, and accordingly, the organic light-emitting device may have improved emission efficiency.

In an embodiment, an amount of Sensitizer B in the emission layer may be about 5 wt % to about 50 wt %, for example, about 10 wt % to about 30 wt %. When the amount is within the range described above, effective energy transfer in the emission layer may be achieved. Thus, the organic light-emitting device may have high efficiency and long lifespan.

In an embodiment, an amount of the heterocyclic compound in the emission layer may be about 0.01 wt % to about 15 wt %, for example, about 0.05 wt % to about 3 wt %, but embodiments are not limited thereto.

In an embodiment, the heterocyclic compound may further satisfy Condition 5:

0 μs<T_{decay}(HC)<5 μs Condition 5
wherein, in Condition 5,
T_{decay}(HC) indicates a decay time of the heterocyclic compound.

The decay time of the heterocyclic compound is a value calculated from a time-resolved photoluminescence (TRPL) spectrum at room temperature of a film (hereinafter, referred to as “Film (HC)”) having a thickness of about 40 nm, which is obtained by vacuum-codepositing the host and the heterocyclic compound included in the emission layer on a quartz substrate at a weight ratio of 90:10 at a vacuum pressure of 10⁻⁷ torr.

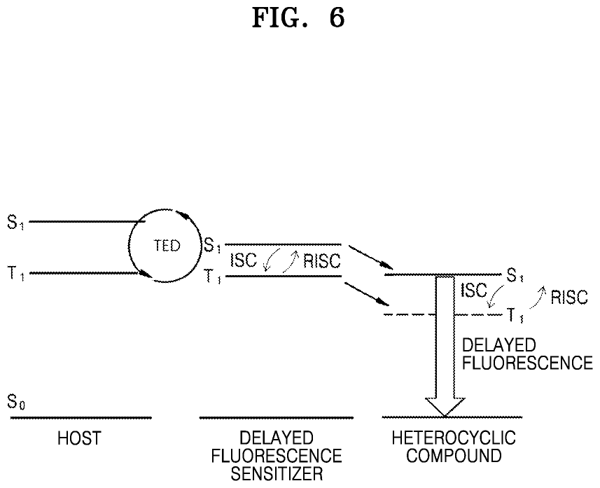

### Description of FIG. 6

In an embodiment, the heterocyclic compound may be used as a delayed fluorescence emitter, and the emission layer may further include a sensitizer, for example, a delayed fluorescence sensitizer.

In an embodiment, the emission layer may further include a host (hereinafter, referred to as ‘Host E’, and Host E is not identical to the heterocyclic compound and a sensitizer) and a sensitizer (hereinafter, referred to as ‘Sensitizer C’, and Sensitizer C is not identical to Host E and the heterocyclic compound). Host E and Sensitizer C may respectively be understood by referring to the description of a host material and a sensitizer material described below, but embodiments are not limited thereto.

In an embodiment, among total emission components emitted from the emission layer, a proportion of emission components of the heterocyclic compound may be greater than or equal to about 80%, for example, greater than or equal to about 90% (or for example, greater than or equal to about 95%). For example, the heterocyclic compound may emit fluorescence and/or delayed fluorescence. In addition, Host E and Sensitizer C may not each emit light.

Here, the heterocyclic compound emits fluorescence and/or delayed fluorescence, and the emission components of the heterocyclic compound are a total of prompt emission components of the heterocyclic compound and delayed fluorescence components of the heterocyclic compound by RISC.

Referring to FIG. 6, energy transfer according to an embodiment is described as follows.

25% of singlet excitons are formed in Host E in the emission layer and are transferred to a singlet of Sensitizer C through FRET, and 75% of triplet excitons are formed in Host E and energy thereof is transferred to a triplet of Sensitizer C. Then, the singlet energy of Sensitizer C is transferred to the heterocyclic compound through FRET, and the triplet energy of Sensitizer C is transferred to the heterocyclic compound through Dexter energy transfer. The energy transferred to the triplet of Sensitizer C may be transferred to the singlet by RISC. In addition, in the case of Sensitizer C, the energy of the triplet state formed in Sensitizer C is reversely transferred to Host E (triplet exciton distributing (TED)), and then transferred to the heterocyclic compound to emit light through RISC.

Accordingly, by transferring all the singlet excitons and triplet excitons generated in the emission layer to a dopant (for example, an emitter), the organic light-emitting device having improved efficiency may be obtained. In addition, since the organic light-emitting device with significantly reduced energy loss is obtained, lifespan characteristics of the organic light-emitting device may be improved.

In an embodiment, when the emission layer further includes Host E and Sensitizer C, in addition to the heterocyclic compound, Host E, Sensitizer C, and the heterocyclic compound may satisfy Condition E-1, E-2, and/or E-3:

S₁(H_{E})≥S₁(S_{C}) Condition E-1

S₁(S_{C})≥S₁(HC) Condition E-2

T₁(S_{C})≥T₁(HC) Condition E-3
wherein, in Conditions E-1, E-2, and E-3,
S₁(H_{E}) indicates a lowest excited singlet energy level of Host E,
S₁(S_{C}) indicates a lowest excited singlet energy level of Sensitizer C,
S₁(HC) indicates a lowest excited singlet energy level of the heterocyclic compound,
T₁(S_{C}) indicates a lowest excited triplet energy level of Sensitizer C, and T₁(HC) indicates a lowest excited triplet energy level of the heterocyclic compound.

S₁(HE), S₁(S_{C}), S₁(HC), T₁(S_{C}), and T₁(HC) are evaluated using the DFT method of the Gaussian program, wherein structure optimization is performed at B3LYP/6-31G(d,p) level.

When Host E, Sensitizer C, and the heterocyclic compound satisfy Condition E-1, E-2, and/or E-3, Dexter energy transfer and FRET from Sensitizer C to the heterocyclic compound may be facilitated, and accordingly, the organic light-emitting device may have improved emission efficiency.

In an embodiment, an amount of Sensitizer C in the emission layer may be about 5 wt % to about 50 wt %, for example, about 10 wt % to about 30 wt %. When the amount is within the range described above, effective energy transfer in the emission layer may be achieved. Thus, the organic light-emitting device may have high efficiency and long lifespan.

In an embodiment, an amount of the heterocyclic compound in the emission layer may be about 0.01 wt % to about 15 wt %, for example, about 0.05 wt % to about 3 wt %, but embodiments are not limited thereto.

### [Host in Emission Layer]

In an embodiment, the host may not include a metal atom.

In an embodiment, the host may include at least one compound of a fluorene-containing compound, a carbazole-containing compound, a dibenzofuran-containing compound, a dibenzothiophene-containing compound, an indenocarbazole-containing compound, an indolocarbazole-containing compound, a benzofurocarbazole-containing compound, a benzothienocarbazole-containing compound, an acridine-containing compound, a dihydroacridine-containing compound, a triindolobenzene-containing compound, a pyridine-containing compound, a pyrimidine-containing compound, a triazine-containing compound, a silicon-containing compound, a cyano group-containing compound, a phosphine oxide-containing compound, a sulfoxide-containing compound, a sulfonyl-containing compound, or any combination thereof.

For example, the host may be a compound including at least one carbazole ring and at least one cyano group, or a phosphine oxide-containing compound.

In an embodiment, the host may consist of one kind of host. When the host consists of one kind of host, the one kind of host may be a bipolar host, an electron-transporting host, or a hole-transporting host, which will be described later.

In an embodiment, the host may be a mixture of two or more different kinds of hosts. For example, the host may be a mixture of an electron-transporting host and a hole-transporting host, a mixture of two different kinds of electron-transporting hosts, or a mixture of two different kinds of hole-transporting hosts. The electron-transporting host and the hole-transporting host may respectively be the same as those described below.

In an embodiment, the host may include an electron-transporting host including at least one electron-transporting moiety and a hole-transporting host that does not include an electron-transporting moiety.

The electron-transporting moiety used herein may be a cyano group, a π electron-deficient nitrogen-containing cyclic group, a group represented by one of the following Formulae, or a combination thereof:

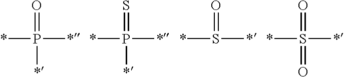

wherein, in Formulae, *, *′, and *″ may each indicate a binding site to any neighboring atom.

In an embodiment, the electron-transporting host in the emission layer may include at least one of a cyano group, a π electron-deficient nitrogen-containing cyclic group, or a combination thereof.

In an embodiment, the electron-transporting host in the emission layer may include at least one cyano group.

In an embodiment, the electron-transporting host in the emission layer may include at least one cyano group and at least one π electron-deficient nitrogen-containing cyclic group.

In an embodiment, the host may include an electron-transporting host and a hole-transporting host, wherein the electron-transporting host may include at least one π electron-deficient nitrogen-free cyclic group and at least one electron-transporting moiety, and the hole-transporting host may include at least one π electron-deficient nitrogen-free cyclic group and may not include an electron-transporting moiety.

The term “π electron-deficient nitrogen-containing cyclic group” used herein refers to a cyclic group having at least one *—N=*′ moiety, and for example, may be selected from: an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, and an azacarbazole group; and a condensed ring in which two or more π electron-deficient nitrogen-containing cyclic groups are condensed with each other.

Meanwhile, the π electron-deficient nitrogen-free cyclic group may be: a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, a triindolobenzene group, a condensed ring in which two or more π electron-deficient nitrogen-free cyclic groups are condensed with each other, or any combination thereof, but embodiments are not limited thereto.

In an embodiment, when the host is a mixture of an electron-transporting host and a hole-transporting host, then then a weight ratio of the electron-transporting host and the hole-transporting host may be 1:9 to 9:1, for example, 2:8 to 8:2, for example, 4:6 to 6:4, for example, 5:5. When the weight ratio of the electron-transporting host and the hole-transporting host satisfies the range described above, then a balance of hole and electron transport in the emission layer may be achieved.

The host may include at least one of TPBi, TBADN, ADN (also referred to as “DNA”), CBP, CDBP, TCP, mCP, Compound H50, Compound H51, or any combination thereof:

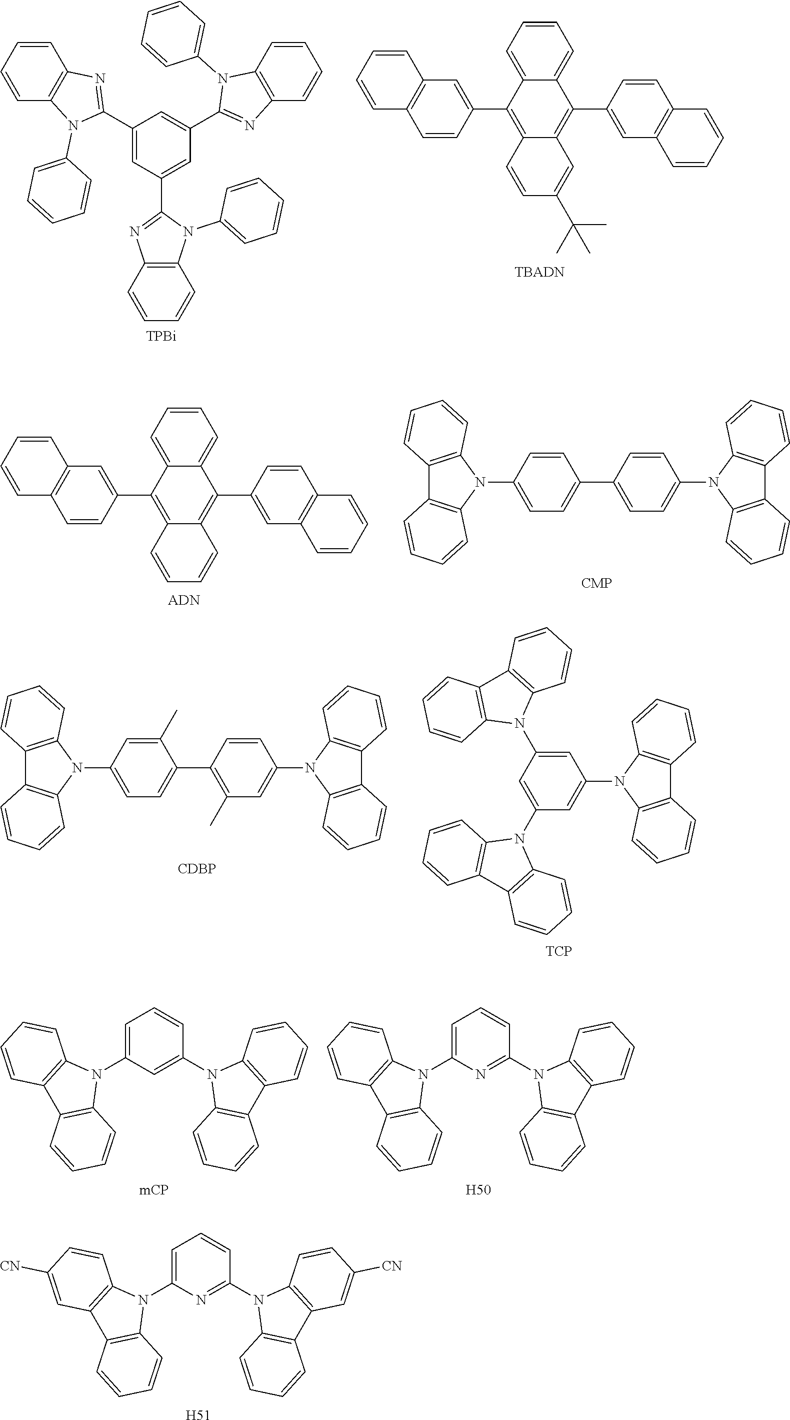

In an embodiment, the host may further include a compound represented by Formula 301:

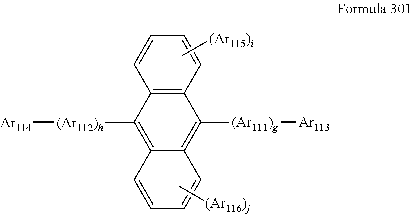

wherein, in Formula 301, Ar₁₁₁ and Ar₁₁₂ may each independently be:
a phenylene group, a naphthalene group, a phenanthrenylene group, or a pyrenylene group; or
a phenylene group, a naphthalene group, a phenanthrenylene group, or a pyrenylene group, each substituted with at least one phenyl group, naphthyl group, anthracenyl group, or any combination thereof.

Ar₁₁₃ to Ar₁₁₆ in Formula 301 may each independently be:
a C₁-C₁₀ alkyl group, a phenyl group, a naphthyl group, a phenanthrenyl group, or a pyrenyl group; or
a phenyl group, a naphthyl group, a phenanthrenyl group, and a pyrenyl group, each substituted with at least one of phenyl group, naphthyl group, anthracenyl group, or any combination thereof.

The designations g, h, i, and j in Formula 301 may each independently be an integer from 0 to 4, and may be, for example, 0, 1, or 2.

In Formula 301, Ar₁₁₃ to Ar₁₁₆ may each independently be:
a C₁-C₁₀ alkyl group that is substituted with at least one of a phenyl group, a naphthyl group, an anthracenyl group, or any combination thereof;
a phenyl group, a naphthyl group, an anthracenyl group, a pyrenyl, a phenanthrenyl group, or a fluorenyl group;
a phenyl group, a naphthyl group, an anthracenyl group, a pyrenyl group, a phenanthrenyl group, or a fluorenyl group, each substituted with at least one deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a phenyl group, a naphthyl group, an anthracenyl group, a pyrenyl group, a phenanthrenyl group, a fluorenyl group; a group shown below, or a combination thereof, but embodiments are not limited thereto.

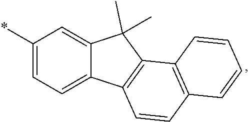 [SMILES: *c1ccc2c(c1)C(C)(C)c1c-2ccc2ccccc12]

In an embodiment, the host may include a compound represented by Formula 302:

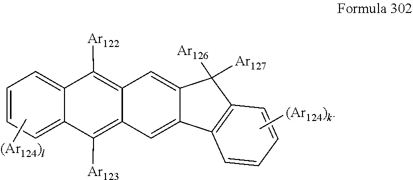

Ar₁₂₂ to Ar₁₂₅ in Formula 302 are the same as described in detail in connection with Ar₁₁₃ in Formula 301.

Ar₁₂₆ and Ar₁₂₇ in Formula 302 may each independently be a C₁-C₁₀ alkyl group (for example, a methyl group, an ethyl group, or a propyl group).

The designations k and l in Formula 302 may each independently be an integer from 0 to 4. For example, k and l may be 0, 1, or 2.

In an embodiment, the host may include at least one of Compounds H1 to H25:

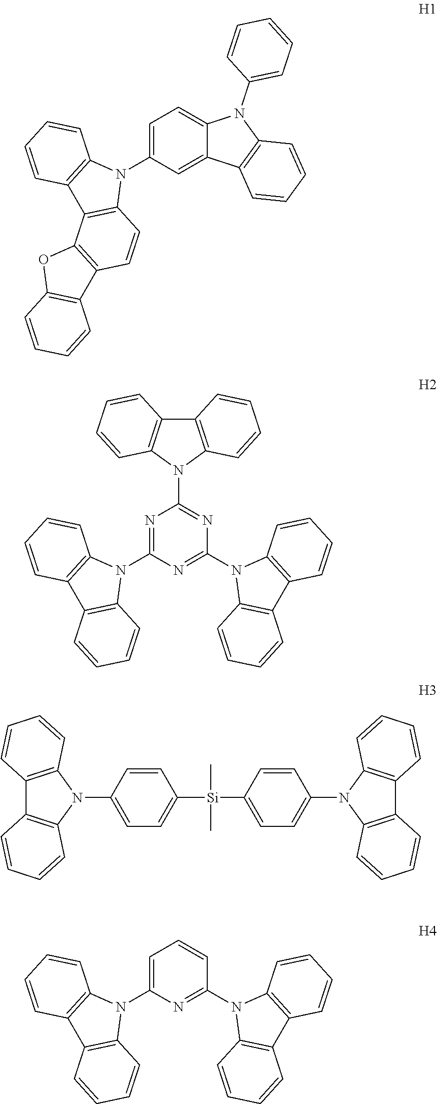 [SMILES: c1ccc(-n2c3ccccc3c3cc(-n4c5ccccc5c5c6oc7ccccc7c6ccc54)ccc32)cc1 ; c1ccc2c(c1)c1ccccc1n2-c1nc(-n2c3ccccc3c3ccccc32)nc(-n2c3ccccc3c3ccccc32)n1 ; C[Si](C)(c1ccc(-n2c3ccccc3c3ccccc32)cc1)c1ccc(-n2c3ccccc3c3ccccc32)cc1 ; c1cc(-n2c3ccccc3c3ccccc32)nc(-n2c3ccccc3c3ccccc32)c1]

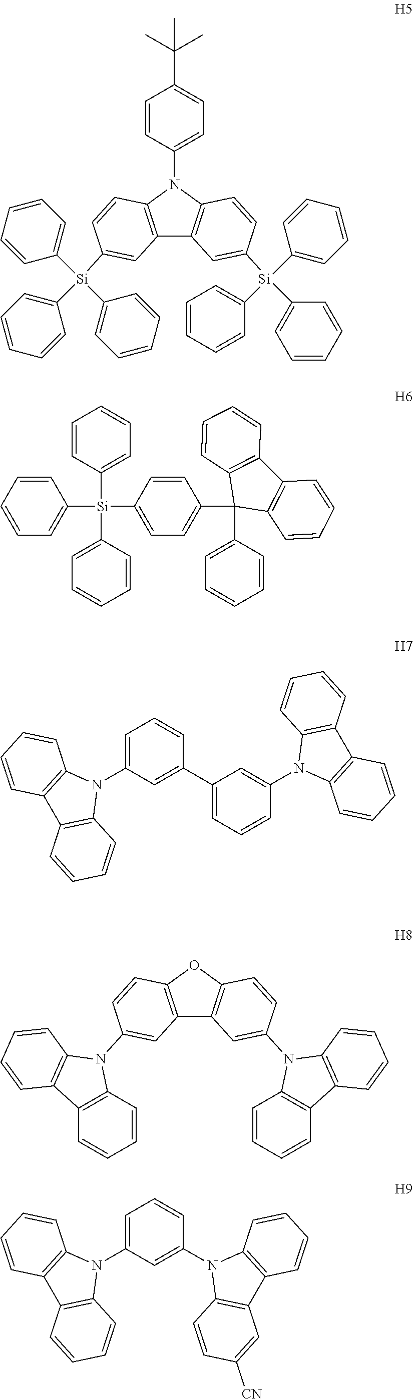 [SMILES: CC(C)(C)c1ccc(-n2c3ccc([Si](c4ccccc4)(c4ccccc4)c4ccccc4)cc3c3cc([Si](c4ccccc4)(c4ccccc4)c4ccccc4)ccc32)cc1 ; c1ccc(C2(c3ccc([Si](c4ccccc4)(c4ccccc4)c4ccccc4)cc3)c3ccccc3-c3ccccc32)cc1 ; c1cc(-c2cccc(-n3c4ccccc4c4ccccc43)c2)cc(-n2c3ccccc3c3ccccc32)c1 ; c1ccc2c(c1)c1ccccc1n2-c1ccc2oc3ccc(-n4c5ccccc5c5ccccc54)cc3c2c1 ; N#Cc1ccc2c(c1)c1ccccc1n2-c1cccc(-n2c3ccccc3c3ccccc32)c1]

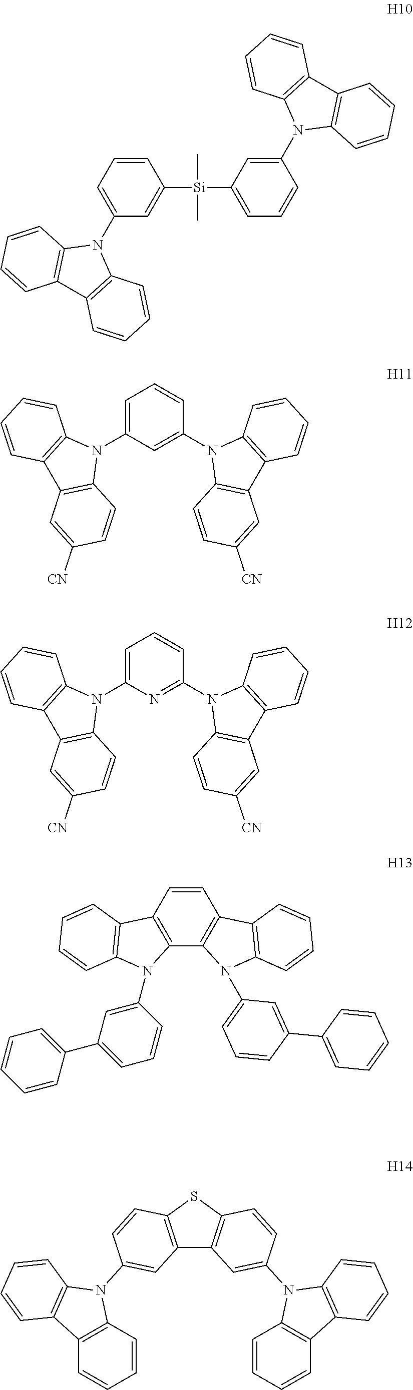

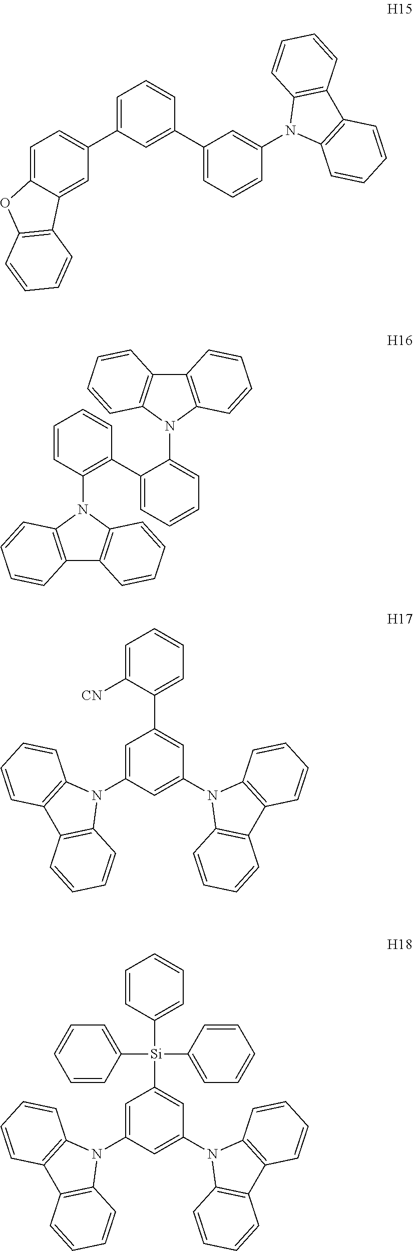

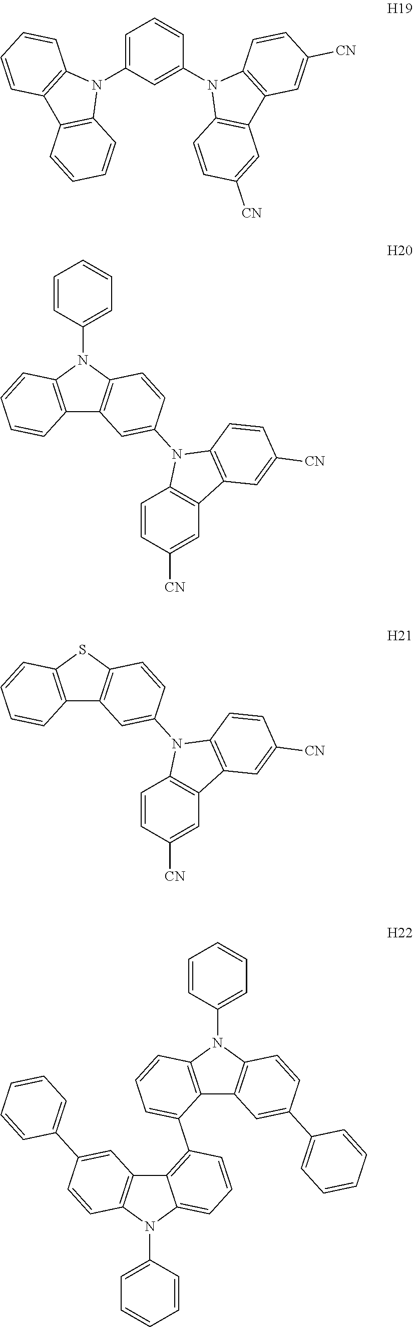 [SMILES: N#Cc1ccc2c(c1)c1cc(C#N)ccc1n2-c1cccc(-n2c3ccccc3c3ccccc32)c1 ; N#Cc1ccc2c(c1)c1cc(C#N)ccc1n2-c1ccc2c(c1)c1ccccc1n2-c1ccccc1 ; N#Cc1ccc2c(c1)c1cc(C#N)ccc1n2-c1ccc2sc3ccccc3c2c1 ; c1ccc(-c2ccc3c(c2)c2c(-c4cccc5c4c4cc(-c6ccccc6)ccc4n5-c4ccccc4)cccc2n3-c2ccccc2)cc1]

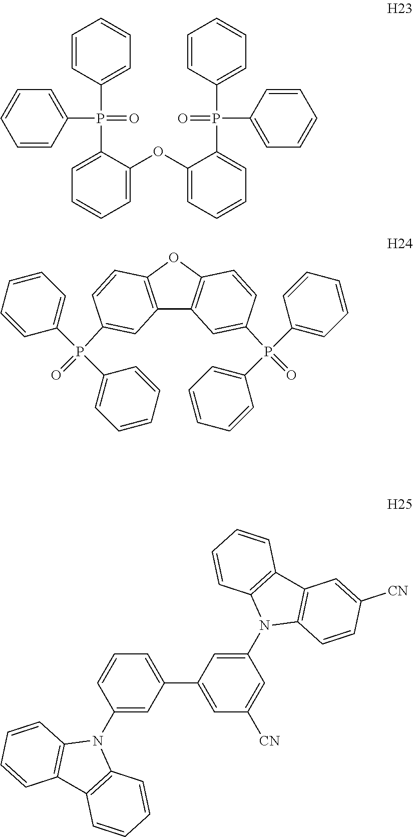 [SMILES: O=P(c1ccccc1)(c1ccccc1)c1ccccc1Oc1ccccc1P(=O)(c1ccccc1)c1ccccc1 ; O=P(c1ccccc1)(c1ccccc1)c1ccc2oc3ccc(P(=O)(c4ccccc4)c4ccccc4)cc3c2c1 ; N#Cc1cc(-c2cccc(-n3c4ccccc4c4ccccc43)c2)cc(-n2c3ccccc3c3cc(C#N)ccc32)c1]

In an embodiment, the host may consist of one kind of compound. For example, the one kind of compound may be a first material (hole-transporting host), a second material (electron-transporting host) as described above, or any combination thereof.

In an embodiment, the host may include two or more kinds of compounds. For example, the host may include two or more different kinds of hole-transporting hosts, or two or more different kinds of electron-transporting hosts, or a combination of at least one hole-transporting host and at least one electron-transporting host.

### [Emitter in Emission Layer]

The emitter includes the heterocyclic compound.

### [Sensitizer in Emission Layer]

In an embodiment, the sensitizer may include a phosphorescent compound.

In an embodiment, the phosphorescent compound may include an organometallic compound including at least one metal.

In an embodiment, the organometallic compound may include: at least one transition metal (M₁₁); and an organic ligand (L₁₁), wherein L₁₁ and M₁₁ may form one cyclometalated ring, two cyclometalated rings, three cyclometalated rings, or four cyclometalated rings.

In an embodiment, the organometallic compound may be represented by Formula 101:

M₁₁(L₁₁)ₙ₁₁(L₁₂)ₙ₁₂. Formula 101
wherein, in Formula 101
M₁₁ may be a transition metal, and
L₁₁ may be a ligand represented by one of Formulae 1-1 to 1-4; and
L₁₂ may be a monodentate ligand or a bidentate ligand,
n11 may be 1, and
n12 may be selected from 0, 1, and 2,

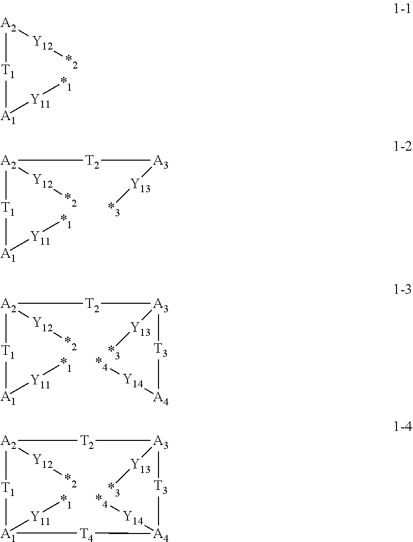

wherein, in Formulae 1-1 to 1-4,
A₁ to A₄ may each independently be a substituted or unsubstituted C₅-C₃₀ carbocyclic group, a substituted or unsubstituted C₁-C₃₀ heterocyclic group, or a non-cyclic group,
Y₁₁ to Y₁₄ may each independently be a chemical bond, O, S, N(R₉₁), B(R₉₁), P(R₉₁), or C(R₉₁)(R₉₂),
T₁ to T₄ may each independently be a single bond, a double bond, *—N(R₉₃)—*, *—B(R₉₃)—*′, *—P(R₉₃)—*′, *—C(R₉₃)(R₉₄)—*′, *—S₁(R₉₃)(R₉₄)—*′, *—Ge(R₉₃)(R₉₄)—*′, *—S—*′, *—Se—*′, *—O—*′, *—C(═O)—*′, *—S(═O)—*′, *—S(═O)₂—*, *—C(R₉₃)=*′, *═C(R₉₃)—*′, *—C(R₉₃)═C(R₉₄)—*, *—C(═S)—*′, or *—C≡C—*′,
a substituent of the substituted C₅-C₃₀ carbocyclic group, a substituent of the substituted C₁-C₃₀ heterocyclic group, and R₉₁ to R₉₄ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent aromatic condensed polycyclic group, a substituted or unsubstituted monovalent aromatic condensed heteropolycyclic group, a substituted or unsubstituted monovalent non-aromatic condensed polycyclic group, a substituted or unsubstituted monovalent non-aromatic condensed heteropolycyclic group, —S₁(Q₁)(Q₂)(Q₃), —Ge(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂), wherein the substituent of the substituted C₅-C₃₀ carbocyclic group and the substituent of the substituted C₁-C₃₀ heterocyclic group are not hydrogen,
*₁, *₂, *₃, and *₄ each indicate a binding site to M₁₁, and
Q₁ to Q₃ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ alkylheteroaryl group, a C₁-C₆₀ heteroaryloxy group, a C₁-C₆₀ heteroarylthio group, a monovalent aromatic condensed polycyclic group, a monovalent aromatic condensed heteropolycyclic group, a monovalent non-aromatic condensed polycyclic group, a monovalent non-aromatic condensed heteropolycyclic group, a C₁-C₆₀ alkyl group that is substituted with at least one deuterium, —F, a cyano group, a C₁-C₆₀ alkyl group, a C₆-C₆₀ aryl group, or any combination thereof, or a C₆-C₆₀ aryl group that is substituted with at least one deuterium, —F, a cyano group, a C₁-C₆₀ alkyl group, a C₆-C₆₀ aryl group, or any combination thereof.

In an embodiment, the transition metal may be platinum (Pt), palladium (Pd), gold (Au), iridium (Ir), osmium (Os), titanium (T₁), zirconium (Zr), hafnium (Hf), europium (Eu), terbium (Tb), thulium (Tm), or rhodium (Rh).

In an embodiment, the sensitizer may include a delayed fluorescence compound.

In an embodiment, the delayed fluorescence compound may be represented by Formula 101 or 102.

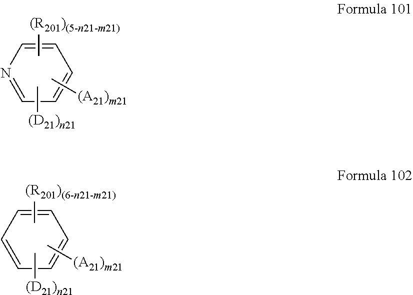

wherein, in Formulae 101 and 102,
A₂₁ may be an acceptor group,
D₂₁ may be a donor group,
m21 may be 1, 2, or 3, n21 may be 1, 2, or, 3, and
the sum of n21 and m21 in Formula 101 may be 5 or less, and the sum of n21 and m21 in Formula 102 may be 6 or less,
R₂₀₁ may be hydrogen, deuterium, —F, —Cl, —Br, —I, —SF₅, a hydroxyl group, a cyano group, a nitro group, an amidino group, a hydrazino group, a hydrazono group, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkylheteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —S₁(Q₁)(Q₂)(Q₃), —Ge(Q₁)(Q₂)(Q₃), —C(Q₁)(Q₂)(Q₃), —B(Q₁)(Q₂), —N(Q₁)(Q₂), —P(Q₁)(Q₂), —C(═O)(Q₁), —S(═O)(Q₁), —S(═O)₂(Q₁), —P(═O)(Q₁)(Q₂), or —P(═S)(Q₁)(Q₂), and a plurality of R₂₀₁(s) may optionally be bonded together to form a substituted or unsubstituted C₅-C₃₀ carbocyclic group or a substituted or unsubstituted C₁-C₃₀ heterocyclic group,
wherein Q₁ to Q₃ may each independently be hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkylaryl group, a C₆-C₆₀ aryloxy group, a C₆-C₆₀ arylthio group, a C₁-C₆₀ heteroaryl group, a C₂-C₆₀ alkylheteroaryl group, a C₁-C₆₀ heteroaryloxy group, a C₁-C₆₀ heteroarylthio group, a monovalent aromatic condensed polycyclic group, a monovalent aromatic condensed heteropolycyclic group, a monovalent non-aromatic condensed polycyclic group, a monovalent non-aromatic condensed heteropolycyclic group, a C₁-C₆₀ alkyl group that is substituted with at least one deuterium, —F, a cyano group, a C₁-C₆₀ alkyl group, a C₆-C₆₀ aryl group, or any combination thereof, or a C₆-C₆₀ aryl group that is substituted with at least one deuterium, —F, a cyano group, a C₁-C₆₀ alkyl group, a C₆-C₆₀ aryl group, or any combination thereof.

In an embodiment, in Formulae 101 and 102, A₂₁ may be a substituted or unsubstituted π electron-deficient nitrogen-free cyclic group.

In detail, the π electron-deficient nitrogen-free cyclic group may be: a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, or a triindolobenzene group; or a condensed ring in which two or more π electron-deficient nitrogen-free cyclic groups are condensed with each other, but embodiments are not limited thereto.

For example, in Formulae 101 and 102, D₂₁ may be: —F, a cyano group, or a π electron-deficient nitrogen-containing cyclic group;
a C₁-C₆₀ alkyl group, a π electron-deficient nitrogen-containing cyclic group, or a π electron-deficient nitrogen-free cyclic group, each substituted with at least one of —F, a cyano group, or any combination thereof; or
a π electron-deficient nitrogen-containing cyclic group, each substituted with at least one of deuterium, a C₁-C₆₀ alkyl group, a π electron-deficient nitrogen-containing cyclic group, or a π electron-deficient nitrogen-free cyclic group.

In detail, the π electron-deficient nitrogen-free cyclic group is the same as described above.

In detail, the π electron-deficient nitrogen-containing cyclic group refers to a cyclic group having at least one *—N=*′ moiety, and, for example, may be: an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, an azacarbazole group, or a benzimidazolobenzimidazole group; or a condensed ring in which two or more π electron-deficient nitrogen-containing cyclic groups are condensed with each other.

In an embodiment, in the organic layer, an amount of the sensitizer may be greater than an amount of the emitter. For example, a volume ratio of the sensitizer and the emitter may be about 30:0.1 to about 10:3, or about 10:0.1 to about 20:5. As another example, a weight ratio of the sensitizer and the emitter may be about 10:0.1 to about 20:5. Meanwhile, a volume ratio of the host and the sensitizer in the organic layer may be about 60:40 to about 95:5 or about 70:30 to about 90:10. Alternatively, a weight ratio of the host and the sensitizer may be about 60:40 to about 95:5. When the amount is within the range described above, the organic light-emitting device may have improved emission efficiency and/or lifespan characteristics.

FIG. 1 is a schematic cross-sectional view of the organic light-emitting device 10 according to an embodiment. Hereinafter, referring to FIG. 1, the structure and manufacturing method of the organic light-emitting device 10 according to an embodiment are described as follows. The organic light-emitting device 10 includes a first electrode 11, an organic layer 15, and a second electrode 19, which are sequentially stacked.

A substrate may be additionally disposed under the first electrode 11 or on the second electrode 19. For use as the substrate, any substrate that is used in organic light-emitting devices available in the art may be used, and for example, a glass substrate or a transparent plastic substrate, each having excellent mechanical strength, thermal stability, transparency, surface smoothness, ease of handling, and water resistance, may be used.

The first electrode 11 may be formed by depositing or sputtering, for example, onto the substrate, a material for forming the first electrode 11. The first electrode 11 may be an anode. The material for forming the first electrode 11 may be a material with a high work function for easy hole injection. The first electrode 11 may be a reflective electrode, a semi-transmissive electrode, or a transmissive electrode. The material for forming the first electrode 11 may be indium tin oxide (ITO), indium zinc oxide (IZO), tin oxide (SnO₂), or zinc oxide (ZnO). Alternatively, the material for forming the first electrode 11 may be metal, such as magnesium (Mg), aluminum (Al), aluminum-lithium (Al—Li), calcium (Ca), magnesium-indium (Mg—In), or magnesium-silver (Mg—Ag).

The first electrode 11 may have a single-layered structure or a multilayer structure including two or more layers. For example, the first electrode 11 may have a three-layered structure of ITO/Ag/ITO, but embodiments are not limited thereto.

The organic layer 15 is disposed above the first electrode 11.

The organic layer 15 may include a hole transport region, an emission layer, or an electron transport region.

The hole transport region may be between the first electrode 11 and the emission layer.

The hole transport region may include a hole injection layer, a hole transport layer, an electron blocking layer, a buffer layer, or any combination thereof.

The hole transport region may include only either a hole injection layer or a hole transport layer. Alternatively, the hole transport region may have a hole injection layer/hole transport layer structure, a hole injection layer/hole transport layer/electron blocking layer structure, or a hole injection layer/a first hole transport layer/a second hole transport layer/an electron blocking layer structure, wherein, for each structure, respective layers are sequentially stacked in this stated order from the first electrode 11.

When the hole transport region includes a hole injection layer, the hole injection layer may be formed on the first electrode 11 using one or more suitable methods, for example, vacuum deposition, spin coating, casting, and/or Langmuir-Blodgett (LB) deposition.

When a hole injection layer is formed by vacuum deposition, the deposition condition may vary according to a compound used as a material to form the hole injection layer, and the structure and thermal characteristics of the hole injection layer. For example, the deposition condition may include a deposition temperature of about 100° C. to about 500° C., a vacuum pressure of about 10⁻⁸ torr to about 10⁻³ torr, and a deposition rate of about 0.01 Å/sec to about 100 Å/sec. However, the deposition condition are not limited thereto.

When the hole injection layer is formed by spin coating, the coating condition may vary according to a compound used as a material to form the hole injection layer, and the structure and thermal characteristics of the hole injection layer. For example, the coating condition may include a coating speed of about 2,000 rpm to about 5,000 rpm, and a heat treatment temperature of about 80° C. to about 200° C. for removing a solvent after coating. However, the coating condition are not limited thereto.

The conditions for forming the hole transport layer and the electron blocking layer may be the same as the conditions for forming the hole injection layer.

The hole transport region may include at least one of m-MTDATA, TDATA, 2-TNATA, NPB, β-NPB, TPD, spiro-TPD, spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), a compound represented by Formula 201, a compound represented by Formula 202, or any combination thereof:

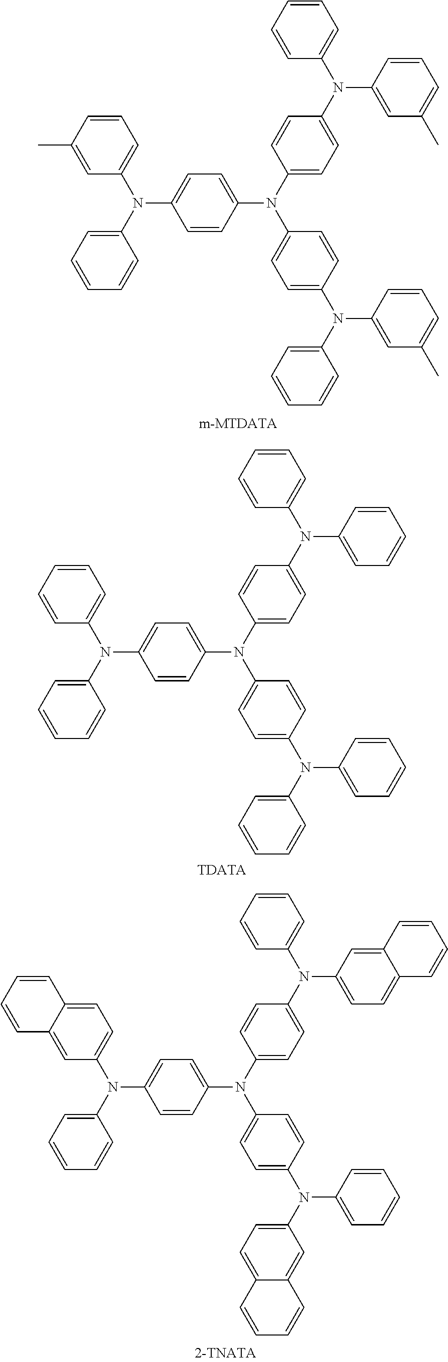

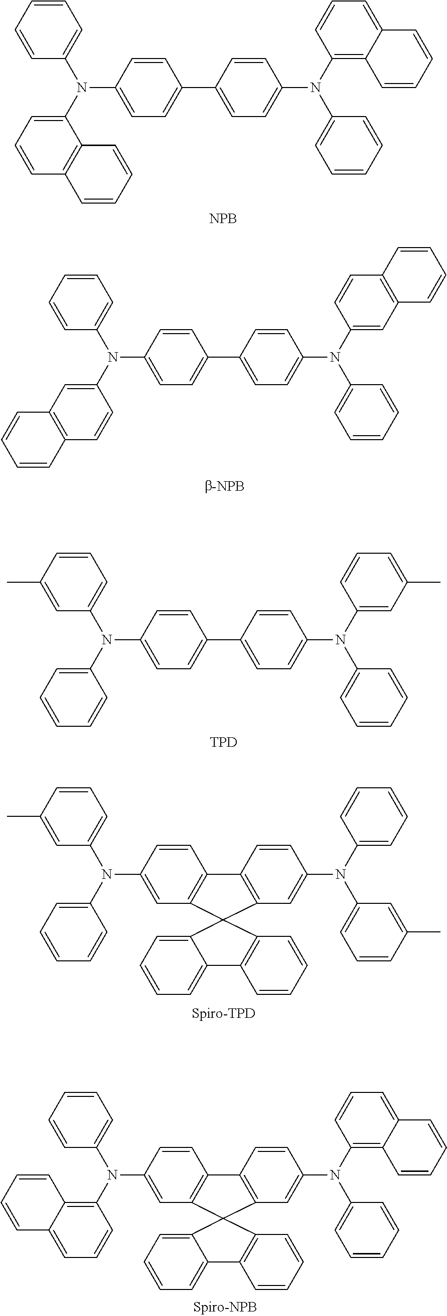

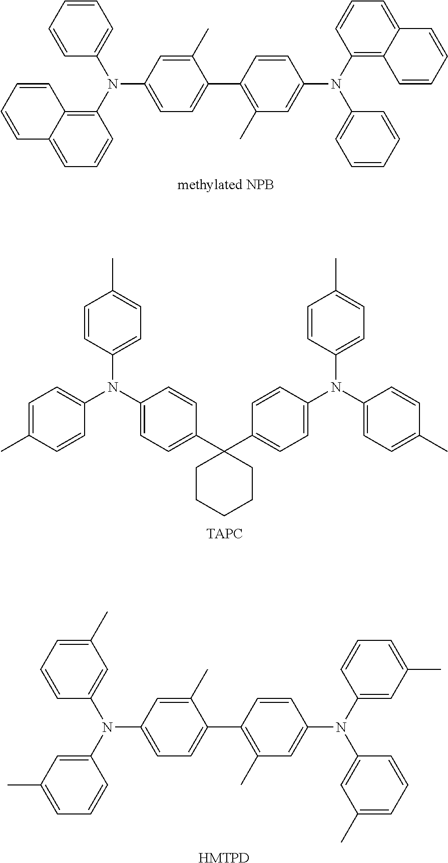

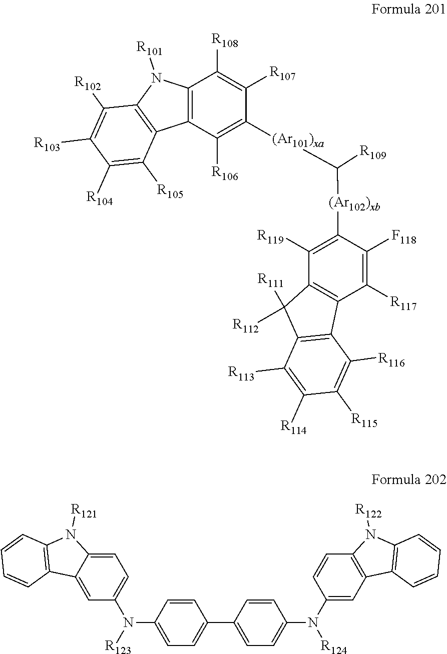

wherein, in Formula 201, Ar₁₀₁ and Ar₁₀₂ may each independently be:
a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, or a pentacenylene group; or
a phenylene group, a pentalenylene group, an indenylene group, a naphthylene group, an azulenylene group, a heptalenylene group, an acenaphthylene group, a fluorenylene group, a phenalenylene group, a phenanthrenylene group, an anthracenylene group, a fluoranthenylene group, a triphenylenylene group, a pyrenylene group, a chrysenylene group, a naphthacenylene group, a picenylene group, a perylenylene group, or a pentacenylene group, each substituted with deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₃-C₁₀ cycloalkenyl group, a C₁-C₁₀ heterocycloalkyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, or any combination thereof.

In Formula 201, xa and xb may each independently be an integer from 0 to 5, or may each independently be or 0, 1, or 2. For example, xa may be 1 and xb may be 0, but xa and xb are not limited thereto.

In Formulae 201 and 202, R₁₀₁ to R₁₀₈, R₁₁₁ to R₁₁₉, and R₁₂₁ to R₁₂₄ may each independently be:
hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group (for example, a methyl group, an ethyl group, a propyl group, a butyl group, a pentyl group, or a hexyl group), or a C₁-C₁₀ alkoxy group (for example, a methoxy group, an ethoxy group, a propoxy group, a butoxy group, or a pentoxy group);
a C₁-C₁₀ alkyl group or a C₁-C₁₀ alkoxy group, each substituted with deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, or a combination thereof;
a phenyl group, a naphthyl group, an anthracenyl group, a fluorenyl group, or a pyrenyl group; or
a phenyl group, a naphthyl group, an anthracenyl group, a fluorenyl group, or a pyrenyl group, each substituted with deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₁₀ alkyl group, a C₁-C₁₀ alkoxy group, or a combination thereof, but embodiments are not limited thereto.

In Formula 201, R₁₀₉ may be:
a phenyl group, a naphthyl group, an anthracenyl group, or a pyridinyl group; or
a phenyl group, a naphthyl group, an anthracenyl group, or a pyridinyl group, each substituted with deuterium, —F, —Cl, —Br, —I, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₂₀ alkyl group, a C₁-C₂₀ alkoxy group, a phenyl group, a naphthyl group, an anthracenyl group, a pyridinyl group, or any combination thereof.

In an embodiment, the compound represented by Formula 201 may be represented by Formula 201A, but embodiments are not limited thereto:

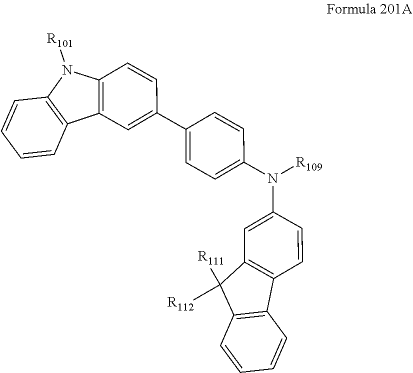

wherein, in Formula 201A, R₁₀₁, R₁₁₁, R₁₁₂, and R₁₀₉ are each the same as described above.

For example, the compound represented by Formula 201 and the compound represented by Formula 202 may include Compounds HT1 to HT20, but embodiments are not limited thereto:

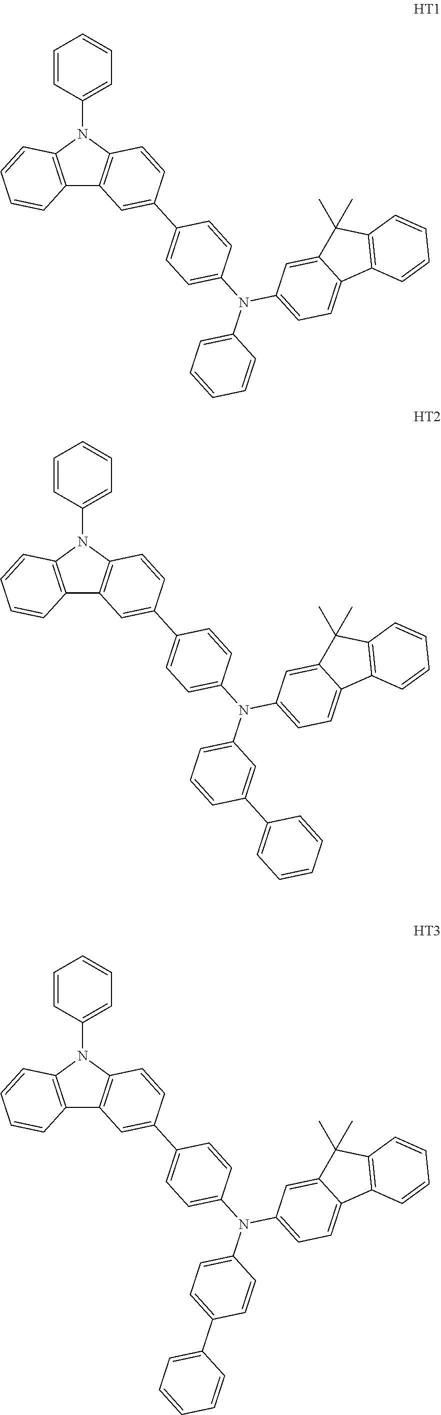 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21]

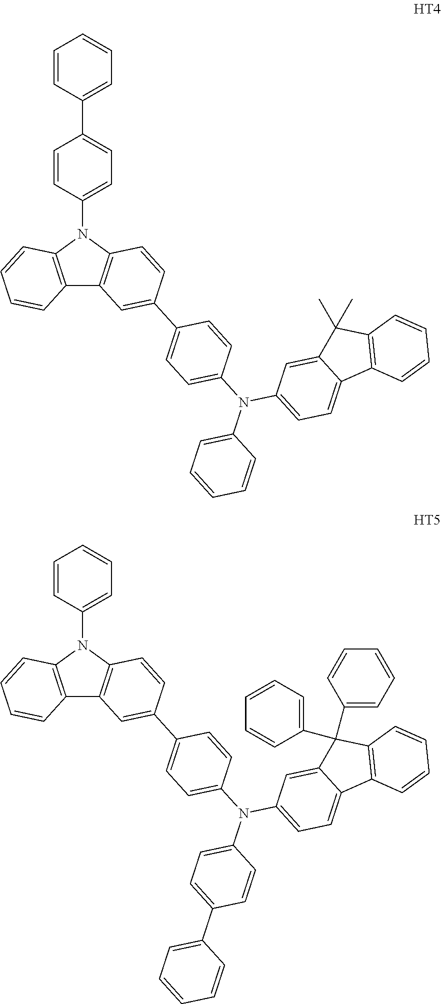 [SMILES: CC1(C)c2ccccc2-c2ccc(N(c3ccccc3)c3ccc(-c4ccc5c(c4)c4ccccc4n5-c4ccc(-c5ccccc5)cc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

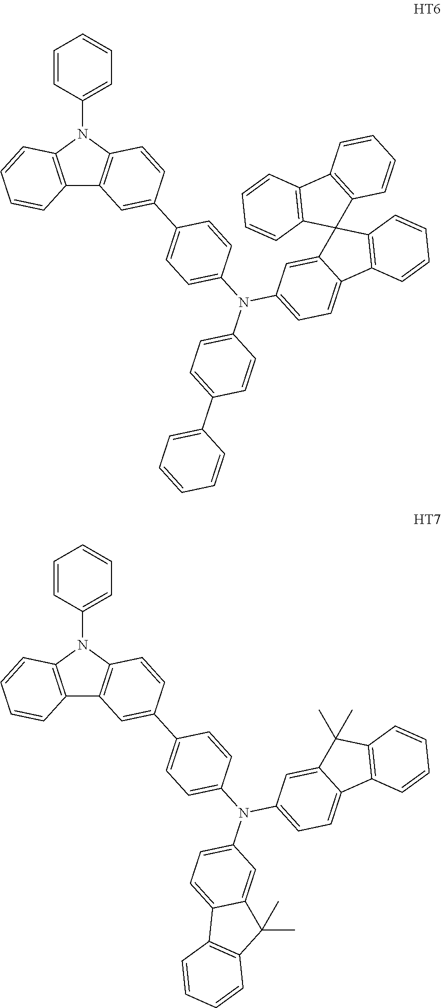 [SMILES: c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21]

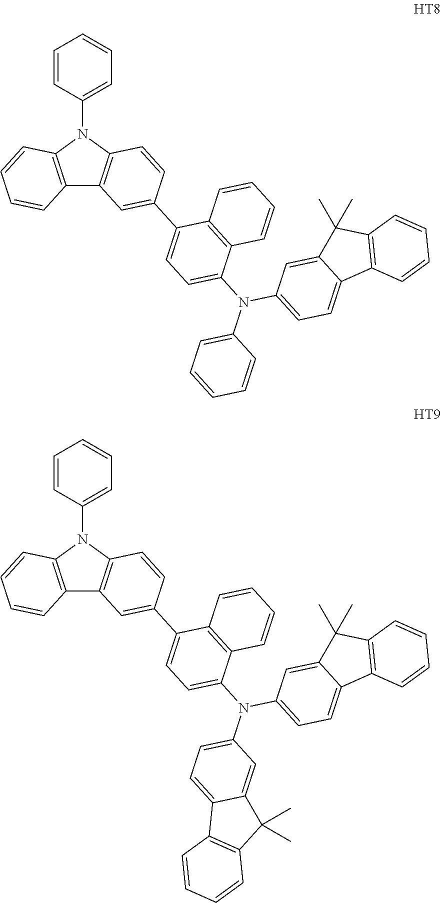 [SMILES: CC1(C)c2ccccc2-c2ccc(N(c3ccccc3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc(-c4ccc5c(c4)c4ccccc4n5-c4ccccc4)c4ccccc34)cc21]

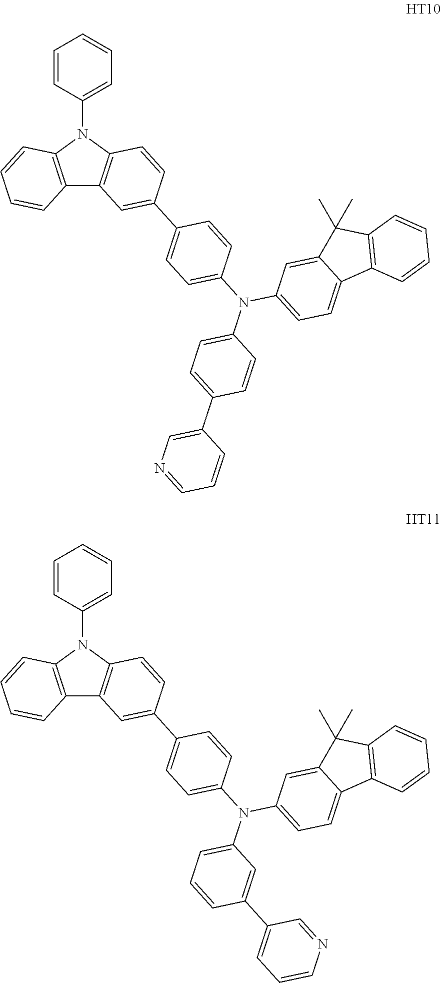 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21]

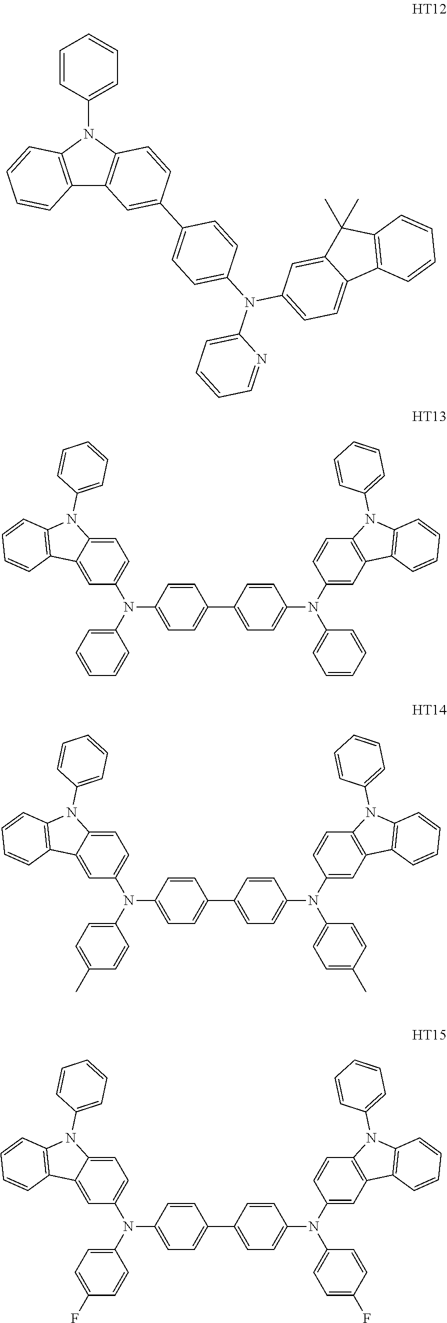 [SMILES: CC1(C)c2ccccc2-c2ccc(N(c3ccc(-c4ccc5c(c4)c4ccccc4n5-c4ccccc4)cc3)c3ccccn3)cc21 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; Cc1ccc(N(c2ccc(-c3ccc(N(c4ccc(C)cc4)c4ccc5c(c4)c4ccccc4n5-c4ccccc4)cc3)cc2)c2ccc3c(c2)c2ccccc2n3-c2ccccc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

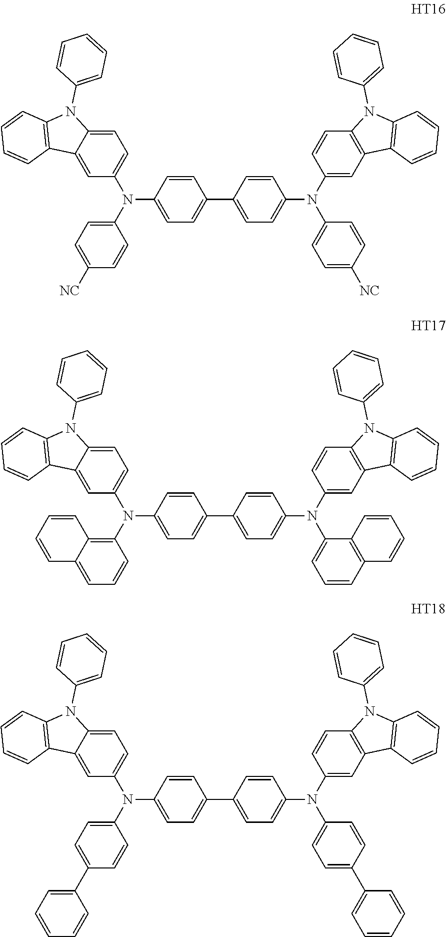

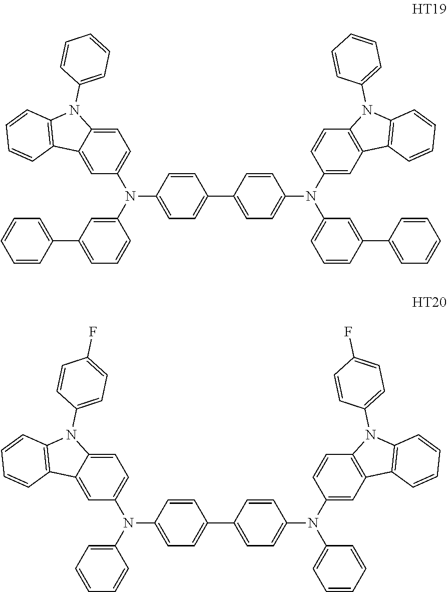 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; Fc1ccc(-n2c3ccccc3c3cc(N(c4ccccc4)c4ccc(-c5ccc(N(c6ccccc6)c6ccc7c(c6)c6ccccc6n7-c6ccc(F)cc6)cc5)cc4)ccc32)cc1]

A thickness of the hole transport region may be about 100 Å to about 10,000 Å, for example, about 100 Å to about 1,000 Å. When the hole transport region includes at least one of a hole injection layer and a hole transport layer, a thickness of the hole injection layer may be about 100 Å to about 10,000 Å, for example, about 100 Å to about 1,000 Å, and a thickness of the hole transport layer may be about 50 Å to about 2,000 Å, for example, about 100 Å to about 1,500 Å. When the thicknesses of the hole transport region, the hole injection layer, and the hole transport layer are within these ranges as described above, satisfactory hole transporting characteristics may be obtained without a substantial increase in driving voltage.

The hole transport region may further include, in addition to these materials, a charge-generation material for the improvement of conductive properties. The charge-generation material may be homogeneously or non-homogeneously dispersed in the hole transport region.

The charge-generation material may be, for example, a p-dopant. The p-dopant may be one of a quinone derivative, a metal oxide, and a cyano group-containing compound, but embodiments are not limited thereto. For example, examples of the p-dopant are: a quinone derivative, such as tetracyanoquinonedimethane (TCNQ) and 2,3,5,6-tetrafluoro-tetracyano-1,4-benzoquinonedimethane (F4-TCNQ); a metal oxide, such as a tungsten oxide or a molybdenum oxide; or a cyano group-containing compound, such as Compound HT-D1 or F12, but embodiments are not limited thereto:

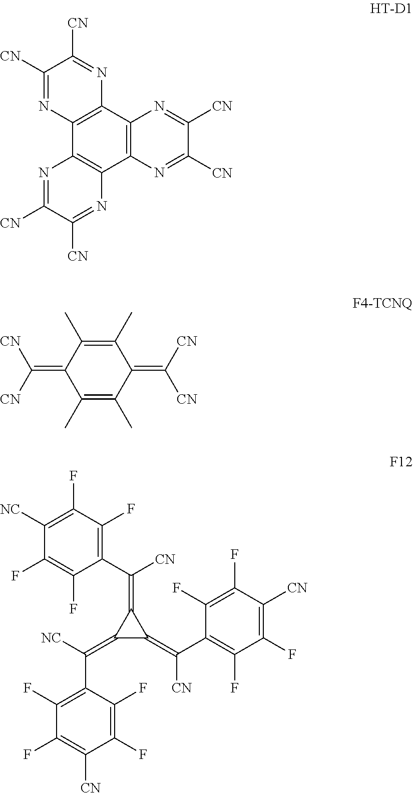

The hole transport region may include a buffer layer.

The buffer layer may compensate for an optical resonance distance according to a wavelength of light emitted from the emission layer, and thus, efficiency of the organic light-emitting device may be improved.

Meanwhile, when the hole transport region includes an electron blocking layer, a material for the electron blocking layer may be selected from materials for the hole transport region described above and materials for a host described above. However, the material for the electron blocking layer is not limited thereto. For example, when the hole transport region includes an electron blocking layer, examples of a material for the electron blocking layer may include mCP described below.

Then, an emission layer may be formed on the hole transport region using vacuum deposition, spin coating, casting, LB deposition, or the like. When the emission layer is formed by vacuum deposition or spin coating, the deposition condition and the coating condition may vary according to a compound used as a material to form the emission layer, but the deposition condition and the coating condition may be similar to those applied in forming the hole injection layer.

When the organic light-emitting device is a full-color organic light-emitting device, the emission layer may be patterned into a red emission layer, a green emission layer, and a blue emission layer. Alternatively, the emission layer has a structure in which a red emission layer, a green emission layer, and/or a blue emission layer are stacked, and thus, may emit white light, and various modifications may be made.

When the emission layer includes a host and a dopant, an amount of the dopant may be about 0.01 parts by weight to about 15 parts by weight based on 100 parts by weight of the host, but embodiments are not limited thereto.

A thickness of the emission layer may be about 100 Å to about 1,000 Å, for example, about 200 Å to about 600 Å. When the thickness of the emission layer is within the range, excellent light-emission characteristics may be obtained without a substantial increase in driving voltage.

Next, an electron transport region may be disposed above the emission layer.

The electron transport region may include a hole blocking layer, an electron transport layer, an electron injection layer, or a combination thereof.

For example, the electron transport region may have a hole blocking layer/electron transport layer/electron injection layer structure or an electron transport layer/electron injection layer structure, but embodiments are not limited thereto. The electron transport layer may have a single-layered structure or a multilayer structure including two or more different materials.

Conditions for forming the hole blocking layer, the electron transport layer, and the electron injection layer which constitute the electron transport region may be understood by referring to the conditions for forming the hole injection layer.

When the electron transport region includes a hole blocking layer, the hole blocking layer may include, for example, at least one of BCP, Bphen, BAlq, or any combination thereof, but embodiments are not limited thereto:

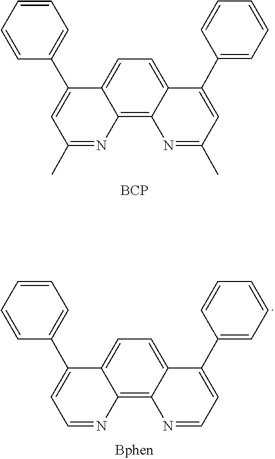

A thickness of the hole blocking layer may be about 20 Å to about 1,000 Å, for example, about 30 Å to about 300 Å. When the thickness of the hole blocking layer is within the range as described above, excellent hole blocking characteristics may be obtained without a substantial increase in driving voltage.

The electron transport layer may include at least one of BCP, Bphen, Alq₃, BAlq, TAZ, NTAZ, or any combination thereof:

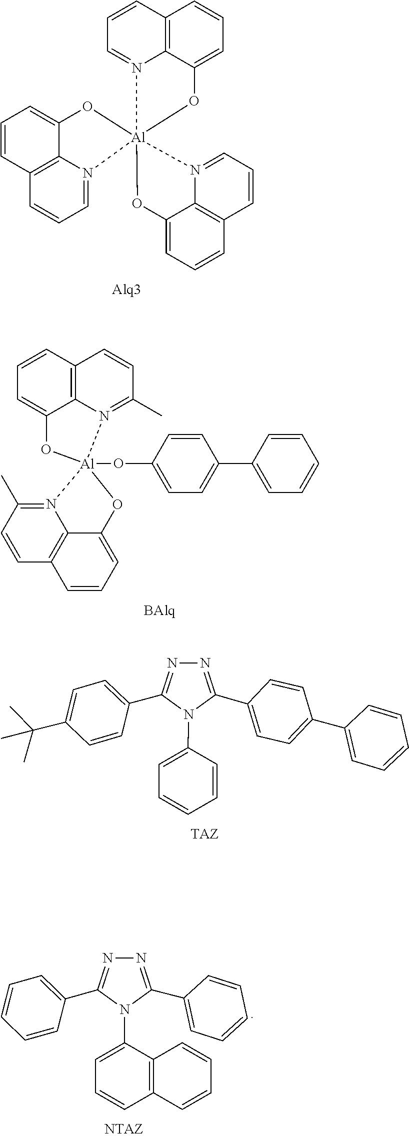

Alternatively, the electron transport layer may include at least one of Compounds ET1 to ET25, but embodiments are not limited thereto:

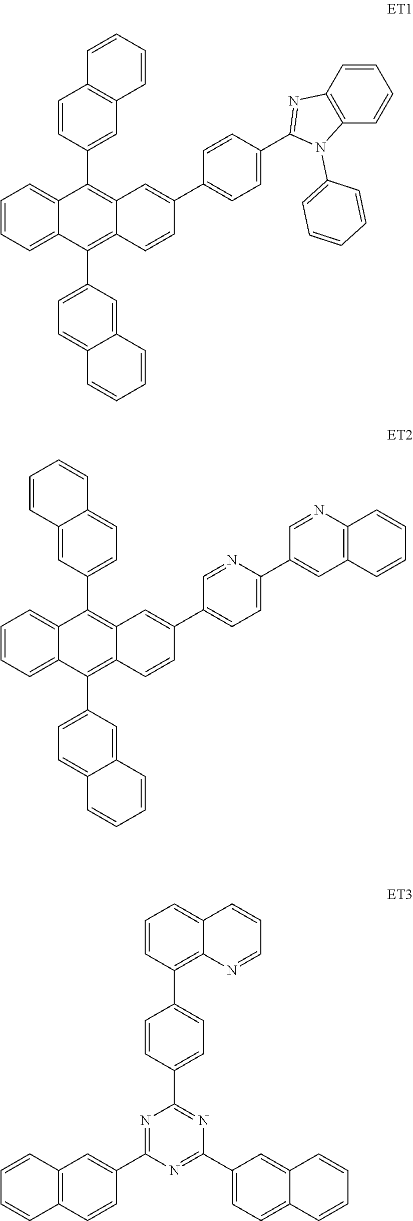 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc2cc(-c3nc(-c4ccc(-c5cccc6cccnc56)cc4)nc(-c4ccc5ccccc5c4)n3)ccc2c1]

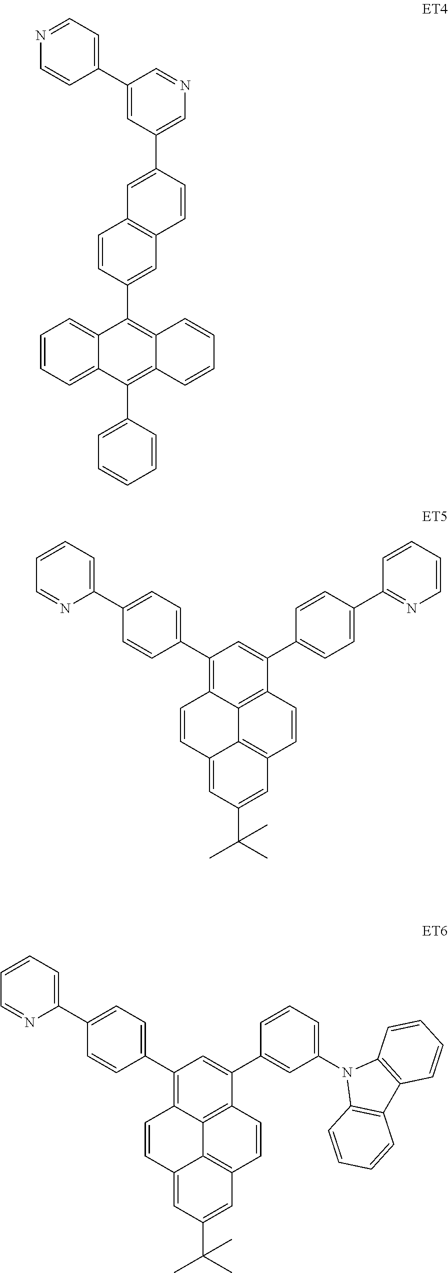 [SMILES: c1ccc(-c2c3ccccc3c(-c3ccc4cc(-c5cncc(-c6ccncc6)c5)ccc4c3)c3ccccc23)cc1 ; CC(C)(C)c1cc2ccc3c(-c4ccc(-c5ccccn5)cc4)cc(-c4ccc(-c5ccccn5)cc4)c4ccc(c1)c2c34 ; CC(C)(C)c1cc2ccc3c(-c4ccc(-c5ccccn5)cc4)cc(-c4cccc(-n5c6ccccc6c6ccccc65)c4)c4ccc(c1)c2c34]

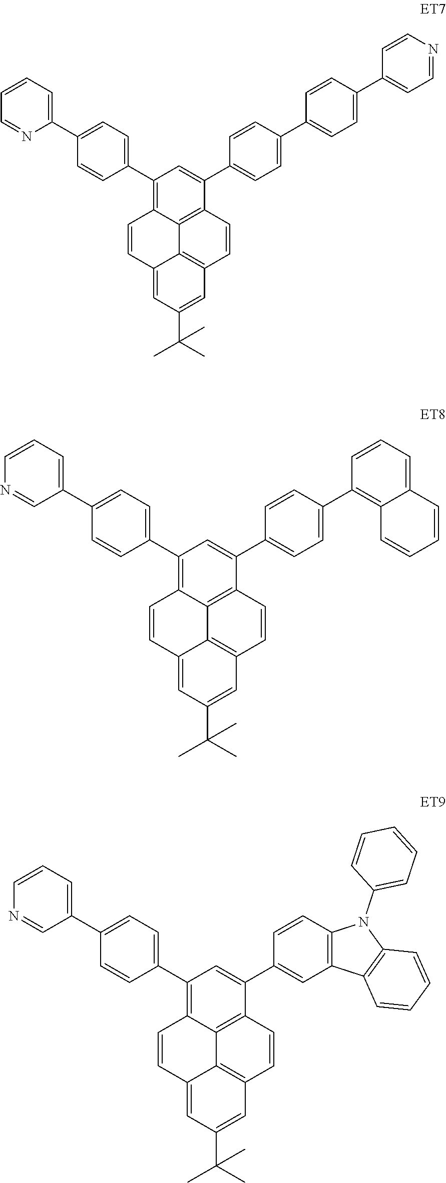 [SMILES: CC(C)(C)c1cc2ccc3c(-c4ccc(-c5ccc(-c6ccncc6)cc5)cc4)cc(-c4ccc(-c5ccccn5)cc4)c4ccc(c1)c2c34 ; CC(C)(C)c1cc2ccc3c(-c4ccc(-c5cccnc5)cc4)cc(-c4ccc(-c5cccc6ccccc56)cc4)c4ccc(c1)c2c34 ; CC(C)(C)c1cc2ccc3c(-c4ccc(-c5cccnc5)cc4)cc(-c4ccc5c(c4)c4ccccc4n5-c4ccccc4)c4ccc(c1)c2c34]

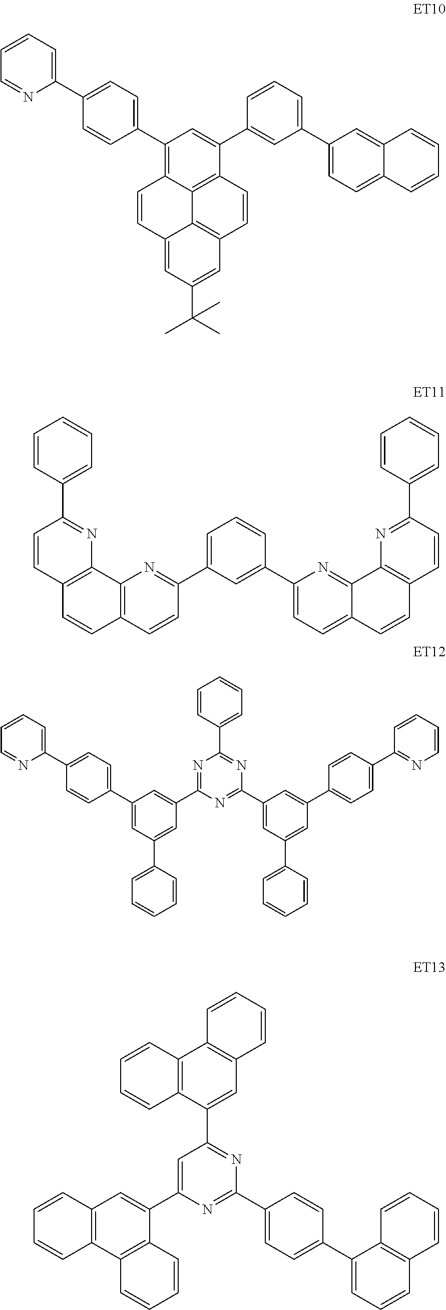 [SMILES: CC(C)(C)c1cc2ccc3c(-c4ccc(-c5ccccn5)cc4)cc(-c4cccc(-c5ccc6ccccc6c5)c4)c4ccc(c1)c2c34 ; c1ccc(-c2ccc3ccc4ccc(-c5cccc(-c6ccc7ccc8ccc(-c9ccccc9)nc8c7n6)c5)nc4c3n2)cc1 ; c1ccc(-c2cc(-c3ccc(-c4ccccn4)cc3)cc(-c3nc(-c4ccccc4)nc(-c4cc(-c5ccccc5)cc(-c5ccc(-c6ccccn6)cc5)c4)n3)c2)cc1 ; c1ccc2c(-c3ccc(-c4nc(-c5cc6ccccc6c6ccccc56)cc(-c5cc6ccccc6c6ccccc56)n4)cc3)cccc2c1]

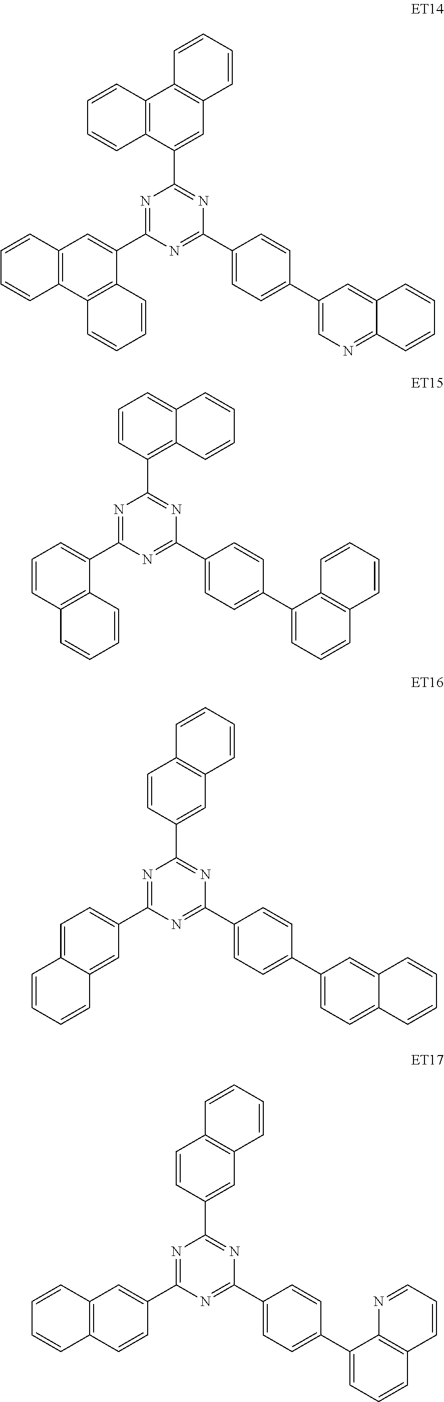 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc2c(-c3ccc(-c4nc(-c5cccc6ccccc56)nc(-c5cccc6ccccc56)n4)cc3)cccc2c1 ; c1ccc2cc(-c3ccc(-c4nc(-c5ccc6ccccc6c5)nc(-c5ccc6ccccc6c5)n4)cc3)ccc2c1 ; c1ccc2cc(-c3nc(-c4ccc(-c5cccc6cccnc56)cc4)nc(-c4ccc5ccccc5c4)n3)ccc2c1]

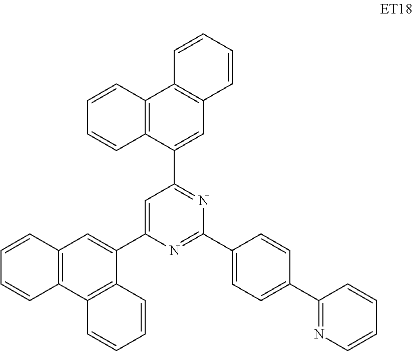 [SMILES: c1ccc(-c2ccc(-c3nc(-c4cc5ccccc5c5ccccc45)cc(-c4cc5ccccc5c5ccccc45)n3)cc2)nc1]

A thickness of the electron transport layer may be about 100 Å to about 1,000 Å, for example, about 150 Å to about 500 Å. When the thickness of the electron transport layer is within the range as described above, satisfactory electron transporting characteristics may be obtained without a substantial increase in driving voltage.

The electron transport layer may include a metal-containing material in addition to the material as described above.

The metal-containing material may include a Li complex. The Li complex may include, for example, Compound ET-D1 (lithium quinolate, LiQ) or ET-D2:

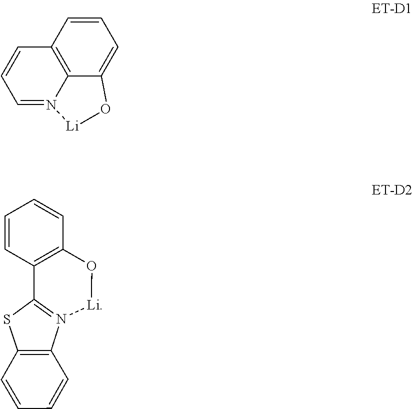

In addition, the electron transport region may include an electron injection layer that facilitates injection of electrons from the second electrode 19 thereinto.

The electron injection layer may include LiF, NaCl, CsF, Li₂O, BaO, or a combination thereof.

A thickness of the electron injection layer may be about 1 Å to about 100 Å, for example, about 3 Å to about 90 Å. When the thickness of the electron injection layer is within the range as described above, satisfactory electron injection characteristics may be obtained without a substantial increase in driving voltage.

The second electrode 19 is disposed above the organic layer 15. The second electrode 19 may be a cathode. A material for forming the second electrode 19 may include a metal, an alloy, an electrically conductive compound, or a combination thereof, which have a relatively low work function. For example, lithium (Li), magnesium (Mg), aluminum (Al), aluminum-lithium (Al—Li), calcium (Ca), magnesium-indium (Mg—In), or magnesium-silver (Mg—Ag) may be used as the material for forming the second electrode 19. Alternatively, a transmissive formed using ITO or IZO may be used as the second electrode 19 in order to manufacture a top-emission type light-emitting device,

Hereinbefore, the organic light-emitting device has been described with reference to FIG. 1, but embodiments are not limited thereto.

According to one or more embodiments, there is provided an electronic apparatus including the organic light-emitting device.

The electronic apparatus may further include a thin-film transistor in addition to the organic light-emitting device as described above. The thin-film transistor may include a source electrode, a drain electrode, and an active layer, wherein any one of the source electrode and the drain electrode may be electrically connected to one of the first electrode and the second electrode of the organic light-emitting device.

According to one or more embodiments, there is provided a diagnostic composition including the heterocyclic compound represented by Formula 1.

The diagnostic composition may include at least one heterocyclic compound represented by Formula 1.

The heterocyclic compound represented by Formula 1 provides high emission efficiency, and accordingly, the diagnostic composition including the heterocyclic compound may have high diagnostic efficiency.

The diagnostic composition may be used in various applications including a diagnosis kit, a diagnosis reagent, a biosensor, and a biomarker.

The C₁-C₆₀ alkyl group as used herein refers to a linear or branched saturated aliphatic hydrocarbon monovalent group having 1 to 60 carbon atoms, and examples thereof include a methyl group, an ethyl group, a propyl group, an isobutyl group, a sec-butyl group, a tert-butyl group, a pentyl group, an isoamyl group, and a hexyl group. The C₁-C₆₀ alkylene group as used herein refers to a divalent group having the same structure as the C₁-C₆₀ alkyl group.

Examples of the C₁-C₆₀ alkyl group, the C₁-C₂₀ alkyl group, and/or the C₁-C₁₀ alkyl group as used herein may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, or a tert-decyl group, each unsubstituted or substituted with a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, or a tert-decyl group. For example, Formula 9-33 is a branched C₆ alkyl group, for example, a tert-butyl group that is substituted with two methyl groups.

The C₁-C₆₀ alkoxy group used herein refers to a monovalent group represented by —OA₁₀₁ (wherein A₁₀₁ is the C₁-C₆ alkyl group), and examples thereof include a methoxy group, an ethoxy group, and an isopropoxy group.

Examples of the C₁-C₆₀ alkoxy group, the C₁-C₂₀ alkoxy group, or the C₁-C₁₀ alkoxy group as used herein may include a methoxy group, an ethoxy group, a propoxy group, a butoxy group, and a pentoxy group.

The C₂-C₆₀ alkenyl group as used herein has a structure including at least one carbon-carbon double bond in the middle or at the terminus of the C₂-C₆₀ alkyl group, and examples thereof include an ethenyl group, a propenyl group, and a butenyl group. The C₂-C₆₀ alkenylene group as used herein refers to a divalent group having the same structure as the C₂-C₆₀ alkenyl group.

The C₂-C₆₀ alkynyl group as used herein has a structure including at least one carbon-carbon triple bond in the middle or at the terminus of the C₂-C₆₀ alkyl group, and examples thereof include an ethynyl group and a propynyl group. The C₂-C₆₀ alkynylene group as used herein refers to a divalent group having the same structure as the C₂-C₆₀ alkynyl group.

The C₃-C₁₀ cycloalkyl group as used herein refers to a monovalent saturated hydrocarbon cyclic group having 3 to 10 carbon atoms, and examples thereof include a cyclopropyl group, a cyclobutyl group, a cyclopentyl group, a cyclohexyl group, and a cycloheptyl group. The C₃-C₁₀ cycloalkylene group as used herein refers to a divalent group having the same structure as the C₃-C₁₀ cycloalkyl group.

The C₃-C₁₀ cycloalkyl group as used herein may include a cyclopropyl group, a cyclobutyl group, a cyclopentyl group, a cyclohexyl group, a cycloheptyl group, a cyclooctyl group, an adamantanyl group, a bicyclo[1.1.1]pentyl group, a bicyclo[2.1.1]hexyl group, a bicyclo[2.2.1]heptyl group (norbornanyl group), a bicyclo[2.2.2]octyl group, and the like.

The C₁-C₁₀ heterocycloalkyl group as used herein refers to a monovalent saturated cyclic group having at least one heteroatom, for example, N, O, P, S₁, B, Ti, S, Se, Te, Ge, or any combination thereof as a ring-forming atom and 1 to 10 carbon atoms, and examples thereof include a tetrahydrofuranyl group and a tetrahydrothiophenyl group.

The C₁-C₁₀ heterocycloalkylene group as used herein refers to a divalent group having the same structure as the C₁-C₁₀ heterocycloalkyl group.

Examples of the C₁-C₁₀ heterocycloalkyl group as used herein may include a silolanyl group, a silinanyl group, a tetrahydrofuranyl group, a tetrahydro-2H-pyranyl group, and a tetrahydrothiophenyl group.

The C₃-C₁₀ cycloalkenyl group as used herein refers to a monovalent cyclic group that has 3 to 10 carbon atoms, at least one carbon-carbon double bond in its ring, and no aromaticity, and examples thereof include a cyclopentenyl group, a cyclohexenyl group, and a cycloheptenyl group. The C₃-C₁₀ cycloalkenylene group as used herein refers to a divalent group having the same structure as the C₃-C₁₀ cycloalkenyl group.

The C₁-C₁₀ heterocycloalkenyl group as used herein refers to a monovalent cyclic group that has at least one hetero atom, for example, N, O, P, S₁, B, Ti, S, Se, Te, Ge, or any combination thereof as a ring-forming atom, 2 to 10 carbon atoms, and at least one carbon-carbon double bond in its ring. Examples of the C₁-C₁₀ heterocycloalkenyl group include a 2,3-dihydrofuranyl group and a 2,3-dihydrothiophenyl group. The C₂-C₁₀ heterocycloalkenylene group as used herein refers to a divalent group having the same structure as the C₁-C₁₀ heterocycloalkenyl group.

The C₆-C₆₀ aryl group as used herein refers to a monovalent group having a carbocyclic aromatic system having 6 to 60 carbon atoms, and the C₆-C₆₀ arylene group as used herein refers to a divalent group having a carbocyclic aromatic system having 6 to 60 carbon atoms. Examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each include two or more rings, the two or more rings may be fused to each other.

The C₇-C₆₀ alkylaryl group as used herein refers to a C₆-C₆₀ aryl group that is substituted with at least one C₁-C₆₀ alkyl group.

The C₁-C₆₀ heteroaryl group as used herein refers to a monovalent group having a cyclic aromatic system that has at least one heteroatom, for example, N, O, P, S₁, B, Ti, S, Se, Te, Ge, or any combination thereof as a ring-forming atom, and 1 to 60 carbon atoms. The C₁-C₆₀ heteroarylene group as used herein refers to a divalent group having a carbocyclic aromatic system that has at least one heteroatom, for example, N, O, P, S₁, B, Ti, S, Se, Te, Ge, or any combination thereof as a ring-forming atom, and 1 to 60 carbon atoms. Examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₆-C₆₀ heteroaryl group and the C₆-C₆₀ heteroarylene group each include two or more rings, the two or more rings may be fused to each other.

The C₂-C₆₀ alkylheteroaryl group as used herein refers to a C₁-C₆₀ heteroaryl group that is substituted with at least one C₁-C₆₀ alkyl group.

The C₆-C₆₀ aryloxy group as used herein indicates —OA₁₀₂ (wherein A₁₀₂ is the C₆-C₆₀ aryl group), and the C₆-C₆₀ arylthio group as used herein indicates —SA₁₀₃ (wherein A₁₀₃ is the C₆-C₆₀ aryl group).

The C₁-C₆₀ heteroaryloxy group used herein indicates —OA₁₀₄ (wherein A₁₀₄ is a C₁-C₆₀ heteroaryl group), and the C₁-C₆₀ heteroarylthio group indicates —SA₁₀₅ (wherein A₁₀₅ is the C₁-C₆ heteroaryl group).

The monovalent non-aromatic condensed polycyclic group as used herein refers to a monovalent group having two or more rings condensed with each other, only carbon atoms (for example, having 8 to 60 carbon atoms) as ring-forming atoms, and no aromaticity in its molecular structure when considered as a whole. Examples of the monovalent non-aromatic condensed polycyclic group include a fluorenyl group. The divalent non-aromatic condensed polycyclic group as used herein refers to a divalent group having the same structure as a monovalent non-aromatic condensed polycyclic group.

The monovalent non-aromatic condensed heteropolycyclic group as used herein refers to a monovalent group having two or more rings condensed with each other, a heteroatom, for example, N, O, P, S₁, B, Ti, S, Se, Te, Ge, or any combination thereof, other than carbon atoms (for example, having 2 to 60 carbon atoms), as a ring-forming atom, and no aromaticity in its molecular structure when considered as a whole. Examples of the monovalent non-aromatic condensed heteropolycyclic group include a carbazolyl group. The divalent non-aromatic condensed heteropolycyclic group as used herein refers to a divalent group having the same structure as a monovalent non-aromatic condensed heteropolycyclic group.

The C₅-C₃₀ carbocyclic group as used herein refers to a saturated or unsaturated cyclic group having, as a ring-forming atom, 5 to 30 carbon atoms only. The C₅-C₃₀ carbocyclic group may be a monocyclic group or a polycyclic group. Examples of the “C₅-C₃₀ carbocyclic group (that is unsubstituted or substituted with at least one R₁ₐ)” may include an adamantane group, a norbornene group, a bicyclo[1.1.1]pentane group, a bicyclo[2.1.1]hexane group, a bicyclo[2.2.1]heptane group (norbornane group), a bicyclo[2.2.2]octane group, a cyclopentane group, a cyclohexane group, a cyclohexene group, a benzene group, a naphthalene group, an anthracene group, a phenanthrene group, a triphenylene group, a pyrene group, a chrysene group, a 1,2,3,4-tetrahydronaphthalene group, a cyclopentadiene group, a silole group, and a fluorene group (each unsubstituted or substituted with at least one R₁ₐ).

The C₁-C₃₀ heterocyclic group as used herein refers to a saturated or unsaturated cyclic group having at least one heteroatom, for example, N, O, S₁, P, B, Ti, S, Se, Te, Ge, or any combination thereof, other than 1 to 30 carbon atoms, as a ring-forming atom. The C₁-C₃₀ heterocyclic group may be a monocyclic group or a polycyclic group. Examples of the “C₁-C₃₀ heterocyclic group (that is unsubstituted or substituted with at least one R₁ₐ)” may include a thiophene group, a furan group, a pyrrole group, a silole group, a borole group, a phosphole group, a selenophene group, a germole group, a benzothiophene group, a benzofuran group, an indole group, an indene group, a benzosilole group, a benzoborole group, a benzophosphole group, a benzoselenophene group, a benzogermole group, a dibenzothiophene group, a dibenzofuran group, a carbazole group, a dibenzosilole group, a dibenzoborole group, a dibenzophosphole group, a dibenzoselenophene group, a dibenzogermole group, a dibenzothiophene 5-oxide group, a 9H-fluoren-9-one group, a dibenzothiophene 5,5-dioxide group, an azabenzothiophene group, an azabenzofuran group, an azaindole group, an azaindene group, an azabenzosilole group, an azabenzoborole group, an azabenzophosphole group, an azabenzoselenophene group, an azabenzogermole group, an azadibenzothiophene group, an azadibenzofuran group, an azacarbazole group, an azafluorene group, an azadibenzosilole group, an azadibenzoborole group, an azadibenzophosphole group, an azadibenzoselenophene group, an azadibenzogermole group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group (each unsubstituted or substituted with at least one R₁ₐ).

Herein, TMS represents *—S₁(CH₃)₃, and TMG represents *—Ge(CH₃)₃.

As used herein, the number of carbons in each group that is substituted (e.g., C₁-C₆₀) excludes the number of carbons in the substituent. For example, a C₁-C₆₀ alkyl group can be substituted with a C₁-C₆₀ alkyl group. The total number of carbons included in the C₁-C₆₀ alkyl group substituted with the C₁-C₆₀ alkyl group is not limited to 60 carbons. In addition, more than one C₁-C₆₀ alkyl substituent may be present on the C₁-C₆₀ alkyl group. This definition is not limited to the C₁-C₆₀ alkyl group and applies to all substituted groups that recite a carbon range.

At least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group may be:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —S₁(Q₁₁)(Q₁₂)(Q₁₃), —Ge(Q₁₁)(Q₁₂)(Q₁₃), —N(Q₁₄)(Q₁₅), —B(Q₁₆)(Q₁₇), —P(═O)(Q₁₈)(Q₁₉), or any combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —S₁(Q₂₁)(Q₂₂)(Q₂₃), —Ge(Q₂₁)(Q₂₂)(Q₂₃), —N(Q₂₄)(Q₂₅), —B(Q₂₆)(Q₂₇), —P(═O)(Q₂₈)(Q₂₉), or any combination thereof; or
—S₁(Q₃₁)(Q₃₂)(Q₃₃), —Ge(Q₃₁)(Q₃₂)(Q₃₃), —N(Q₃₄)(Q₃₅), —B(Q₃₆)(Q₃₇), or —P(═O)(Q₃₈)(Q₃₉),
wherein Q₁ to Q, Q₁₁ to Q₁₉, Q₂₁ to Q₂₉, and Q₃₁ to Q₃₉ may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a C₁-C₆₀ heteroaryloxy group, a C₁-C₆₀ heteroarylthio group, a substituted or unsubstituted monovalent non-aromatic condensed polycyclic group, or a substituted or unsubstituted monovalent non-aromatic condensed heteropolycyclic group.

Hereinafter, a compound and an organic light-emitting device according to an embodiment are described in detail with reference to Synthesis Examples and Examples. However, the disclosure is not limited to the following Synthesis Examples and Examples. The wording “B was used instead of A” used in describing Synthesis Examples means that an amount of B used was identical to an amount of A used based on molar equivalence.

### EXAMPLES

### Synthesis Example

### Synthesis Example 1: Synthesis of Compound 1

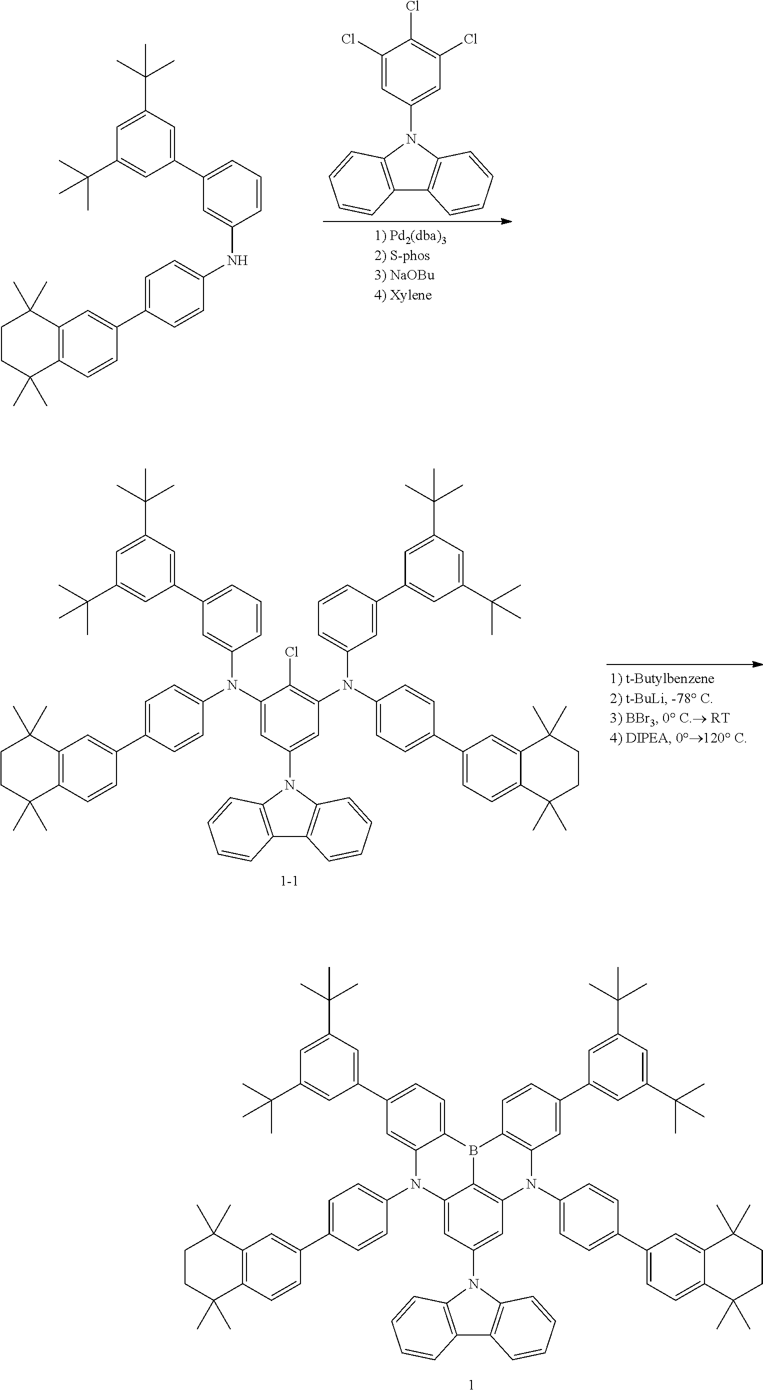

### (1) Synthesis of Compound 1-1

Under nitrogen, 9-(3,4,5-trichlorophenyl)-9H-carbazole (3 g, 8.65 mmol), 3′,5′-di-tert-butyl-N-(4-(5,5,8,8-tetramethyl-5,6,7,8-tetrahydronaphthalen-2-yl)phenyl)-[1,1′-biphenyl]-3-amine (9.69 g, 17.31 mmol), Pd₂(dba)₃ (0.79 g, 0.86 mmol), S-phos (0.710 g, 1.73 mmol), and Nat-OBu (2.91 g, 30.28 mmol) were added to 300 ml of xylene and refluxed for 30 minutes, and then a saturated ammonium chloride solution was used to terminate the reaction. An organic layer was extracted using ethyl acetate and then dried using MgSO₄. The dried organic layer was concentrated in vacuo to remove a solvent therefrom, followed by purification through column chromatography using methylene chloride (MC):hexane (1:3), to thereby obtain 10 g of Compound 1-1 which was a white solid.

LCMS (m/z) calculated: 1361.63 g/mol, found: [M+] 1359.81 g/mol

### (2) Synthesis of Compound 1

Under nitrogen, Compound 1-1 (5 g, 3.6 mmol) was dissolved in 90 ml of t-butylbenzene, the mixture was cooled to −78° C., t-BuLi (6.2 ml, 8.99 mmol) was added thereto, and then the mixture was stirred at 60° C. for 1 hour and 30 minutes. The mixture was cooled to 0° C., boron tribromide (0.69 ml, 7.20 mmol) was added thereto, and then the mixture was stirred at room temperature for an hour and 30 minutes. The mixture was cooled to 0° C., N,N-diisopropylethyl amine (1.22 ml, 7.20 mmol) was added thereto, the mixture was heated at 120° C. for 4 hours, and then a sodium acetate solution (1.0 M in water) was used to terminate the reaction. An organic layer was extracted and dried using MgSO₄. The dried organic layer was concentrated in vacuo to remove a solvent therefrom, followed by purification through column chromatography using MC:hexane (1:4), to thereby obtain 1 g of Compound 1 which was a solid. (yield: 25%)

LCMS (m/z) calculated: 1334.694 g/mol, found: [M⁺] 1333.74 g/mol.

### EXAMPLES

### Example 1

An ITO glass substrate was cut to a size of 50 mm×50 mm×0.5 mm and then, sonicated in acetone isopropyl alcohol and pure water, each for 15 minutes, and then, cleaned by exposure to UV ozone for 30 minutes.

Then, HAT-CN was deposited on the ITO electrode (anode) on the glass substrate to form a hole injection layer having a thickness of 100 Å, NPB was deposited on the hole injection layer to form a first hole transport layer having a thickness of 500 Å, TCTA was deposited on the first hole transport layer to form a second hole transport layer having a thickness of 50 Å, and mCP was deposited on the second hole transport layer to form an electron blocking layer having a thickness of 50 Å.

A first host (H16), a second host (H25), and an emitter (Compound 1) were co-deposited on the electron blocking layer to form an emission layer having a thickness of 400 Å. In this regard, the first host and the second host were mixed at a weight ratio of 60:40, and the emitter was adjusted to be 3 wt % based on the total weight of the first host, the second host, and the emitter.

DBFPO was deposited on the emission layer to form a hole blocking layer having a thickness of 100 Å, and DBFPO and LiQ were co-deposited thereon at a weight ratio of 5:5 to form an electron transport layer having a thickness of 300 Å, LiQ was deposited on the electron transport layer to form an electron injection layer having a thickness of 10 Å, and then Al was deposited on the electron injection layer to form a cathode having a thickness of 1,000 Å, thereby completing the manufacture of an organic light-emitting device.

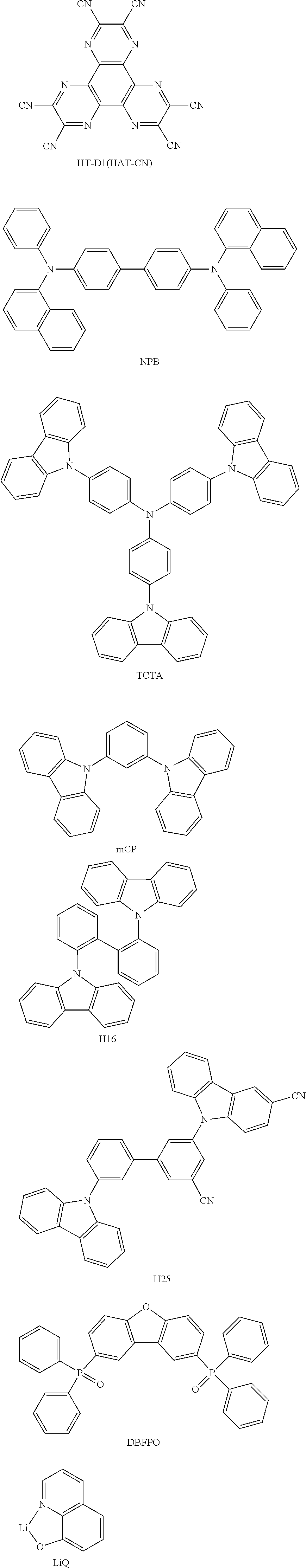

### Comparative Examples 1 and 2

Organic light-emitting devices were manufactured in the same manner as in Example 1, except that compounds shown in Table 2 were each used instead of the emitter in forming the emission layer.

The lifespan T₉₅ (at 1,200 cd/m², hr), FWHM (nm) of EL spectrum, and external quantum efficiency (EQE, %) of the organic light-emitting devices manufactured in Example 1 and Comparative Examples 1 and 2 were measured and evaluated using a current-voltage meter (Keithley 2400) and a luminance meter (Minolta Cs-1000A). The lifespan T₉₅ (at 1,200 cd/m², hr) is a time taken for the luminance of the organic light-emitting devices to reduce from initial luminance of 100% to 95% thereof. The results are shown in Table 2.

TABLE 2

| | Emitter in emission layer | FWHM (nm) | EQE (%) | T₉₅ (hr) |
|---|---|---|---|---|
| Example 1 | Compound 1 | 21 | 21.1 | 48 |
| Comparative Example 1 | Compound A | 22 | 16.3 | 6.1 |
| Comparative Example 2 | Compound B | 23 | 20.7 | 7.1 |

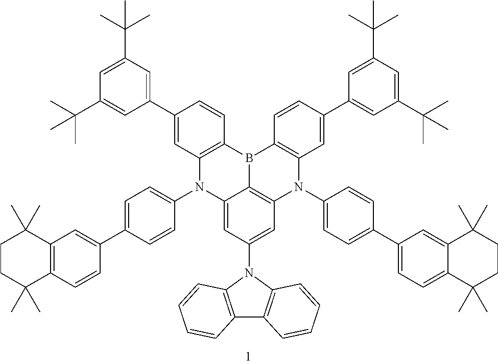 [SMILES: CC(C)(C)c1cc(-c2ccc3c(c2)N(c2ccc(-c4ccc5c(c4)C(C)(C)CCC5(C)C)cc2)c2cc(-n4c5ccccc5c5ccccc54)cc4c2B3c2ccc(-c3cc(C(C)(C)C)cc(C(C)(C)C)c3)cc2N4c2ccc(-c3ccc4c(c3)C(C)(C)CCC4(C)C)cc2)cc(C(C)(C)C)c1]

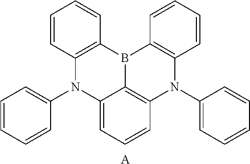

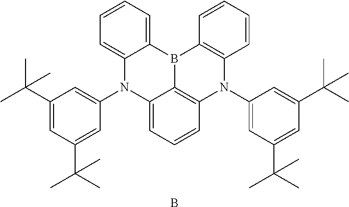

From Table 2, it could be seen that the organic light-emitting device according to an embodiment had excellent emission efficiency, FWHM, and lifespan characteristics. In addition, it could be seen that the organic light-emitting device of Example 1 had higher external quantum efficiency, narrower FWHM, and significantly longer lifespan than those of the organic light-emitting devices of Comparative Examples 1 and 2.

According to the one or more embodiments, a heterocyclic compound has excellent luminescent characteristics and charge mobility characteristics, and thus, an electronic device, for example, an organic light-emitting device, including the heterocyclic compound may have low driving voltage, high efficiency, and long lifespan characteristics. Therefore, a high-quality organic light-emitting device may be implemented using the heterocyclic compound.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A heterocyclic compound represented by Formula 1: 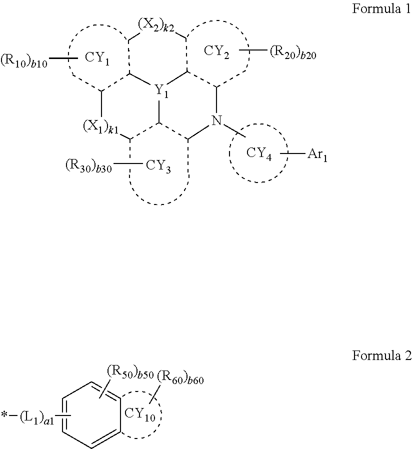
wherein, in Formulae 1 and 2,
Y₁ is B, N, P, P(═O), or P(═S),
X₁ and X₂ are each independently a single bond, O, S, Se, N[(CY₅)—Ar₂], N(R₁), C(R₁)(R₂), S₁(R₁)(R₂), Ge(R₁)(R₂), B(R₁), P(R₁), P(═O)(R₁), S(═O)₂, or C(═O),
k1 is 0 or 1,
k2 is 0 or 1,
the sum of k1 and k2 is greater than or equal to 1,
CY₁ to CY₃ are each independently a C₅-C₃₀ carbocyclic group or a substituted or unsubstituted C₁-C₃₀ heterocyclic group,
CY₄ and CY₅ are each independently a C₅-C₃₀ carbocyclic group that is unsubstituted or substituted with at least one R₄₀ or a C₁-C₃₀ heterocyclic group that is unsubstituted or substituted with at least one R₄₀,
Ar₁ and Ar₂ are each independently a group represented by Formula 2,
L₁ is a single bond, a substituted or unsubstituted C₅-C₃₀ carbocyclic group or a substituted or unsubstituted C₁-C₃₀ heterocyclic group,
a1 is 1, 2, or 3,
CY₁₀ is a C₃-C₁₀ non-aromatic carbocyclic group or a C₁-C₁₀ non-aromatic heterocyclic group,
R₁, R₂, R₁₀, R₂₀, R₃₀, and R₄₀ are each independently a group represented by Formula 2, hydrogen, deuterium, —F, —Cl, —Br, —I, —SF₅, a hydroxyl group, a cyano group, a nitro group, an amidino group, a hydrazino group, a hydrazono group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkylheteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —S₁(Q₁)(Q₂)(Q₃), —Ge(Q₁)(Q₂)(Q₃), —C(═O)(Q₁), —S(═O)(Q₁), —S(═O)₂(Q₁), —B(Q₁)(Q₂), —P(Q₁)(Q₂), —P(═O)(Q₁)(Q₂), —P(═S)(Q₁)(Q₂), or a group represented by Formula 3,
R₅₀ and R₆₀ are each independently hydrogen, deuterium, —F, —Cl, —Br, —I, —SF₅, a hydroxyl group, a cyano group, a nitro group, an amidino group, a hydrazino group, a hydrazono group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkylheteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —S₁(Q₁)(Q₂)(Q₃), —Ge(Q₁)(Q₂)(Q₃), —C(═O)(Q₁), —S(═O)(Q₁), —S(═O)₂(Q₁), —B(Q₁)(Q₂), —P(Q₁)(Q₂), —P(═O)(Q₁)(Q₂), —P(═S)(Q₁)(Q₂), or a group represented by Formula 3, 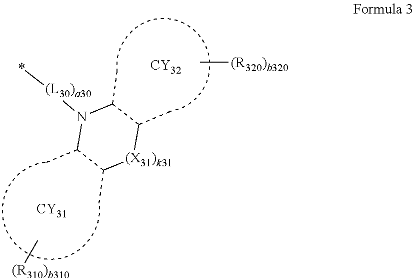
wherein, in Formula 3
X₃₁ is a single bond, O, S, N(R₃₀₁), or C(R₃₀₁)(R₃₀₂),
k31 is 0 or 1,
CY₃₁ and CY₃₂ are each independently a C₅-C₃₀ carbocyclic group or a C₁-C₃₀ heterocyclic group,
L₃₀ is a single bond, a substituted or unsubstituted C₅-C₃₀ carbocyclic group or a substituted or unsubstituted C₁-C₃₀ heterocyclic group,
a30 is 0, 1, 2, or 3,
R₃₀₁, R₃₀₂, R₃₁₀, and R₃₂₀ are each independently hydrogen, deuterium, —F, —Cl, —Br, —I, —SF₅, a hydroxyl group, a cyano group, a nitro group, an amidino group, a hydrazino group, a hydrazono group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkylaryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkylheteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —S₁(Q₁)(Q₂)(Q₃), —Ge(Q₁)(Q₂)(Q₃), —C(═O)(Q₁), —S(═O)(Q₁), —S(═O)₂(Q₁), —B(Q₁)(Q₂), —P(Q₁)(Q₂), —P(═O)(Q₁)(Q₂), or —P(═S)(Q₁)(Q₂),
b310 and b320 are each independently 1, 2, 3, 4, 5, 6, 7, or 8,
b10, b20, b30, b40, and b60 are each independently 1, 2, 3, 4, 5, 6, 7, or 8,
b50 is 1, 2, or 3,
a substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkylaryl group, the substituted C₆-C₆₀ aryloxy group, the substituted C₆-C₆₀ arylthio group, the substituted C₁-C₆₀ heteroaryl group, the C₂-C₆₀ alkylheteroaryl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group is:
deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —S₁(Q₁₁)(Q₁₂)(Q₁₃), —Ge(Q₁₁)(Q₁₂)(Q₁₃), —C(═O)(Q₁₁), —S(═O)(Q₁₁), —S(═O)₂(Q₁₁), —B(Q₁₁)(Q₁₂), —P(Q₁₁)(Q₁₂), —P(═O)(Q₁₁)(Q₁₂), —P(═S)(Q₁₁)(Q₁₂), or any combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —S₁(Q₂₁)(Q₂₂)(Q₂₃), —Ge(Q₂₁)(Q₂₂)(Q₂₃), —C(═O)(Q₂₁), —S(═O)(Q₂₁), —S(═O)₂(Q₂₁), —B(Q₂₁)(Q₂₂), —P(Q₂₁)(Q₂₂), —P(═O)(Q₂₁)(Q₂₂), —P(═S)(Q₂₁)(Q₂₂), or any combination thereof;
—N(Q₃₁)(Q₃₂), —S₁(Q₃₁)(Q₃₂)(Q₃₃), —Ge(Q₃₁)(Q₃₂)(Q₃₃), —C(═O)(Q₃₁), —S(═O)(Q₃₁), —S(═O)₂(Q₃₁), —B(Q₃₁)(Q₃₂), —P(Q₃₁)(Q₃₂), —P(═O)(Q₃₁)(Q₃₂), or —P(Q₃₁)(Q₃₂);
Q₁ to Q₃, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; an amidino group; a hydrazine group; a hydrazone group; a carboxylic acid or a salt thereof; a sulfonic acid or a salt thereof; a phosphoric acid or a salt thereof; a C₁-C₆₀ alkyl group that is unsubstituted or substituted with deuterium, a C₁-C₆₀ alkyl group, a C₆-C₆₀ aryl group, or any combination thereof; a C₂-C₆₀ alkenyl group; a C₂-C₆₀ alkynyl group; a C₁-C₆₀ alkoxy group; a C₃-C₁₀ cycloalkyl group; a C₁-C₁₀ heterocycloalkyl group; a C₃-C₁₀ cycloalkenyl group; a C₁-C₁₀ heterocycloalkenyl group; a C₆-C₆₀ aryl group that is unsubstituted or substituted with deuterium, a C₁-C₆₀ alkyl group, a C₆-C₆₀ aryl group, or any combination thereof; a C₆-C₆₀ aryloxy group; a C₆-C₆₀ arylthio group; a C₁-C₆₀ heteroaryl group; a monovalent non-aromatic condensed polycyclic group; or a monovalent non-aromatic condensed heteropolycyclic group, and
* indicates a binding site to a neighboring atom.

2. The heterocyclic compound of claim 1, wherein the heterocyclic compound is represented by Formula 1A or 1B: 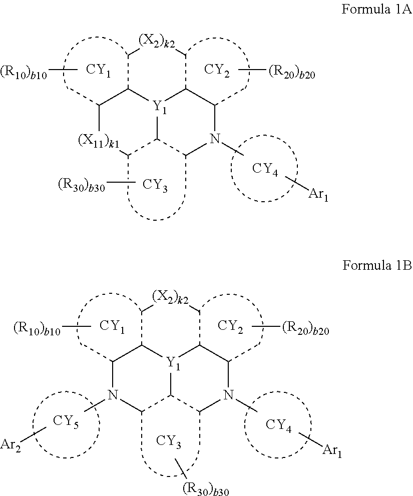
wherein, in Formulae 1A and 1B,
X₁₁ is a single bond, O, S, Se, N(R₁), C(R₁)(R₂), S₁(R₁)(R₂), Ge(R₁)(R₂), B(R₁), P(R₁), P(═O)(R₁), S(═O)₂, or C(═O).

3. The heterocyclic compound of claim 1, wherein CY₁ to CY₃ are each independently a C₆-C₃₀ aromatic carbocyclic group or a C₁-C₃₀ aromatic heterocyclic group.

4. The heterocyclic compound of claim 1, wherein CY₄ and CY₅ are each independently represented by one of Formulae 4-1 to 4-3: 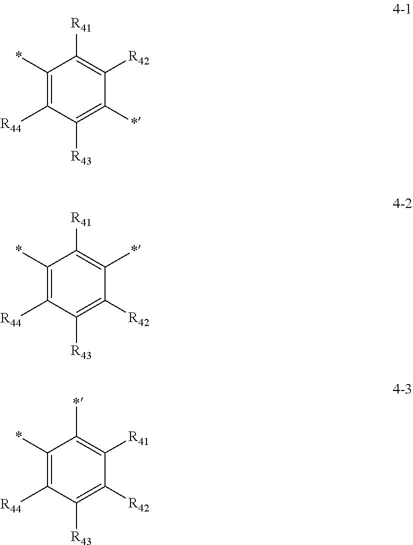
wherein, in Formulae 4-1 to 4-3,
R₄₁ to R₄₄ are each independently the same as described in connection with R₄₀ in claim 1, and
* and *′ each indicate a binding site to a neighboring atom.

5. The heterocyclic compound of claim 1, wherein CY₁₀ is a cyclopentane group, a cyclohexane group, a cycloheptane group, a cyclopentene group, a cyclohexene group, or a cycloheptene group.

6. The heterocyclic compound of claim 1, wherein Ar₁ and Ar₂ are each independently a group represented by Formula 2A or 2B: 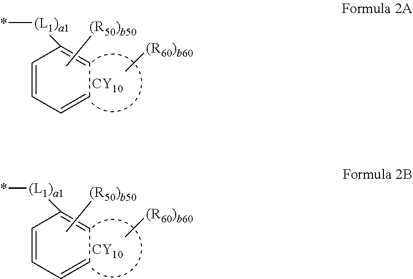
wherein, in Formulae 2A and 2B,
* indicates a binding site to a neighboring atom.

7. The heterocyclic compound of claim 1, wherein Ar₁ and Ar₂ are each independently a group represented by one of Formulae 5-1 to 5-4: 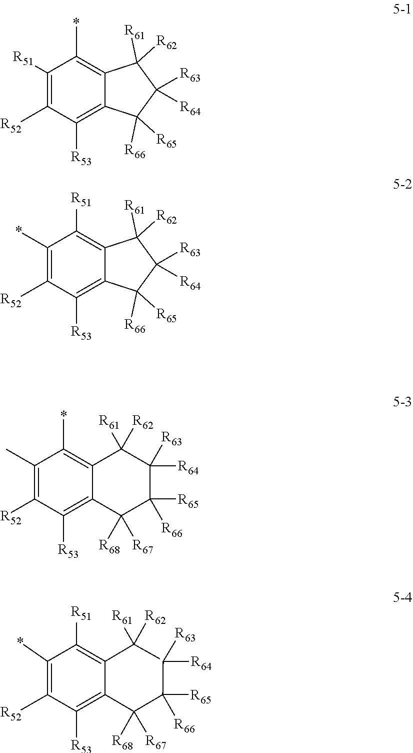
wherein, in Formulae 5-1 to 5-4,
R₅₁ to R₅₃ are each the same as described in connection with R₅₀ in claim 1,
R₆₁ to R₆₈ are each the same as described in connection with R₆₀ in claim 1, and
* indicates a binding site to a neighboring atom.

8. The heterocyclic compound of claim 1, wherein at least one of R₁₀, at least one of R₂₀, and/or at least one of R₃₀ is a group represented by Formula 3.

9. The heterocyclic compound of claim 1, wherein a group represented by Formula 3 is a group represented by one of Formulae 3-1 to 3-3: 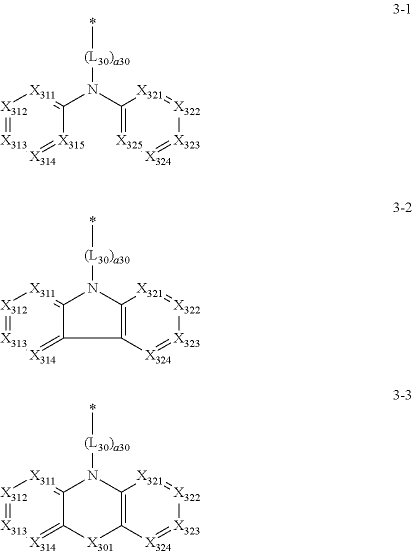
wherein, in Formulae 3-1 to 3-3,
X₃₀₁ is the same as described in connection with X₃₁ in claim 1,
X₃₁₁ is C(R₃₁₁) or N, X₃₁₂ is C(R₃₁₂) or N, X₃₁₃ is C(R₃₁₃) or N, X₃₁₄ is C(R₃₁₄) or N,
X₃₂₁ is C(R₃₂₁) or N, X₃₂₂ is C(R₃₂₂) or N, X₃₂₃ is C(R₃₂₃) or N, X₃₂₄ is C(R₃₂₄) or N,
R₃₁₁ to R₃₁₄ are each independently the same as described in connection with R₃₁₀ in claim 1,
R₃₂₁ to R₃₂₄ are each independently the same as described in connection with R₃₂₀ in claim 1, and
* indicates a binding site to a neighboring atom.

10. The heterocyclic compound of claim 1, wherein the heterocyclic compound is represented by one of Formulae 11-1 to 11-9: 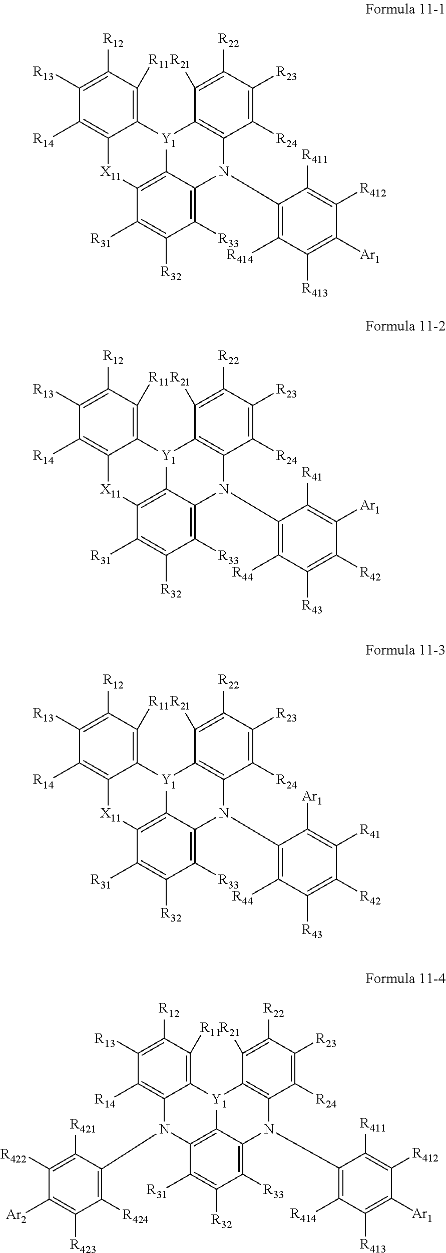 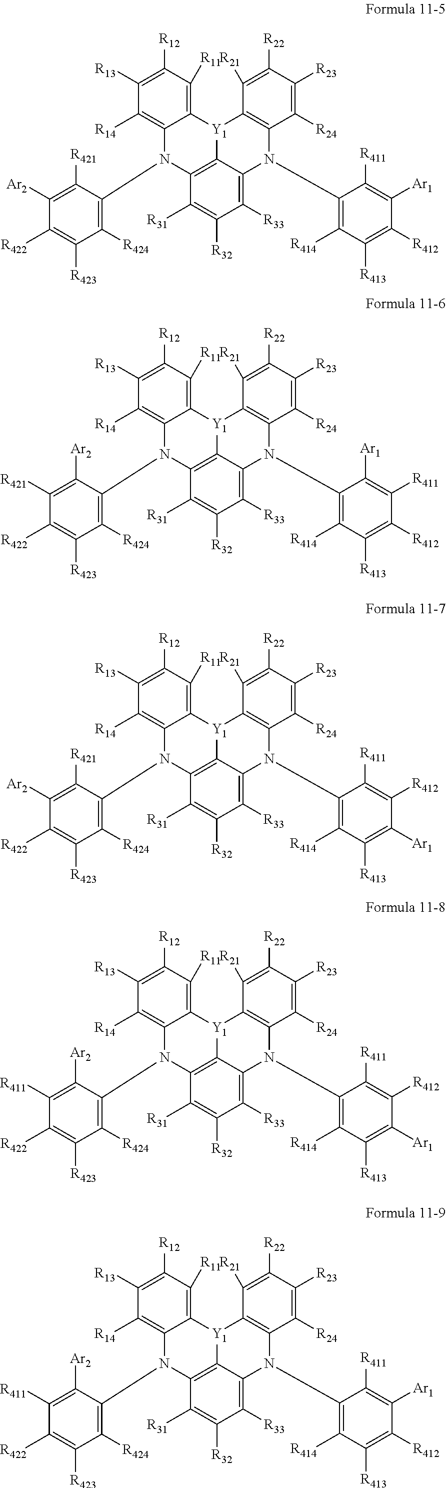
wherein, in Formulae 11-1 to 11-9,
X₁₁ is a single bond, O, S, Se, N(R₁), C(R₁)(R₂), S₁(R₁)(R₂), Ge(R₁)(R₂), B(R₁), P(R₁), P(═O)(R₁), S(═O)₂, or C(═O),
R₁₁ to R₁₄ are each independently the same as described in connection with R₁₀ in claim 1,
R₂₁ to R₂₄ are each independently the same as described in connection with R₂₀ in claim 1,
R₃₁ to R₃₃ are each independently the same as described in connection with R₃₀ in claim 1, and
R₄₁₁ to R₄₁₄ and R₄₂₁ to R₄₂₄ are each independently the same as described in connection with R₄₀ in claim 1.

11. The heterocyclic compound of claim 1, wherein the heterocyclic compound has a symmetric structure or an asymmetric structure.

12. The heterocyclic compound of claim 1, wherein the heterocyclic compound is one of Compounds 1 to 792:

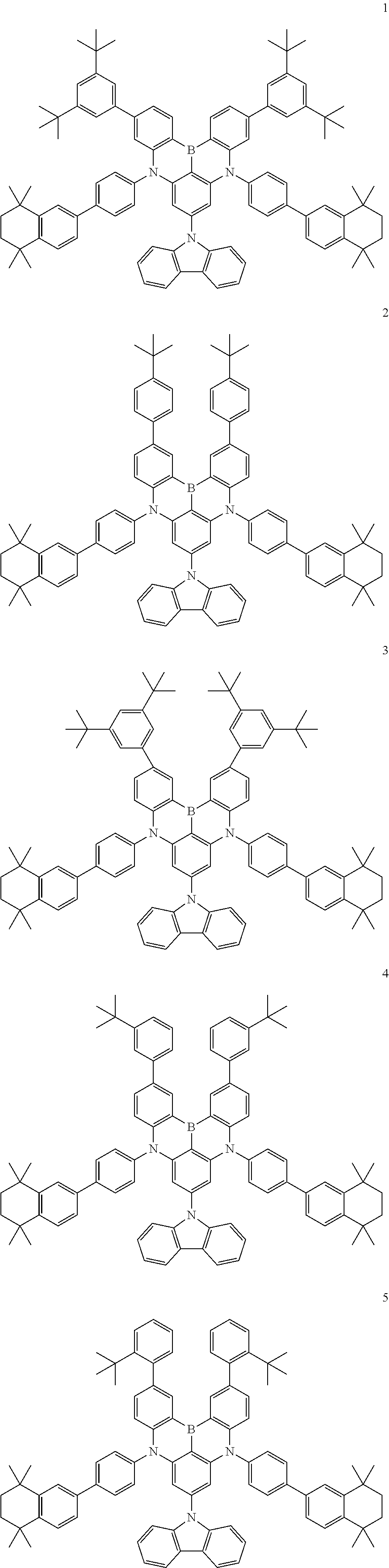

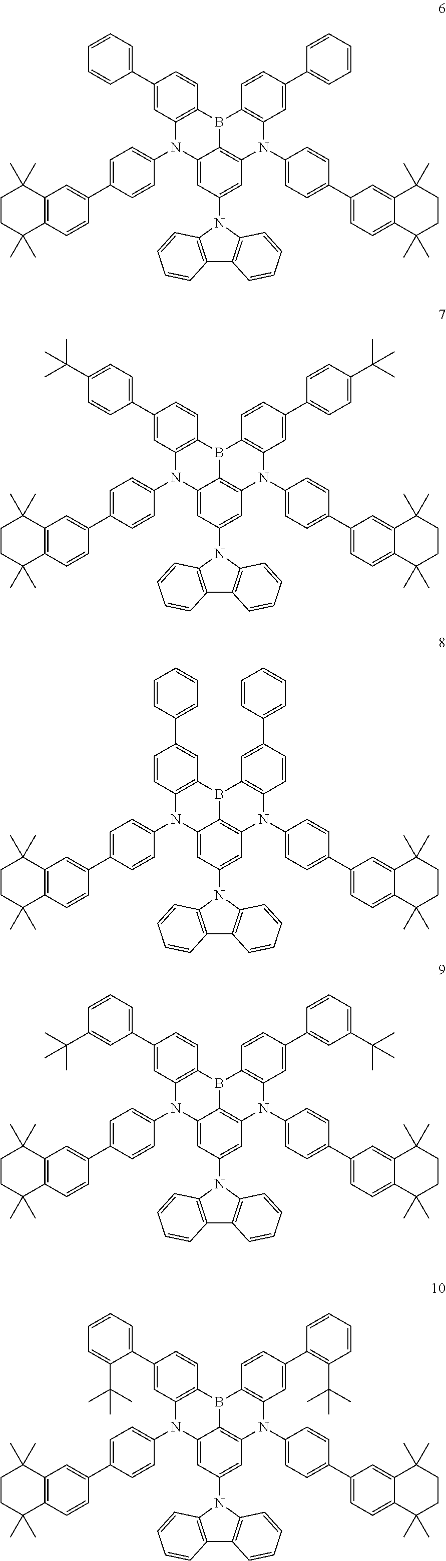

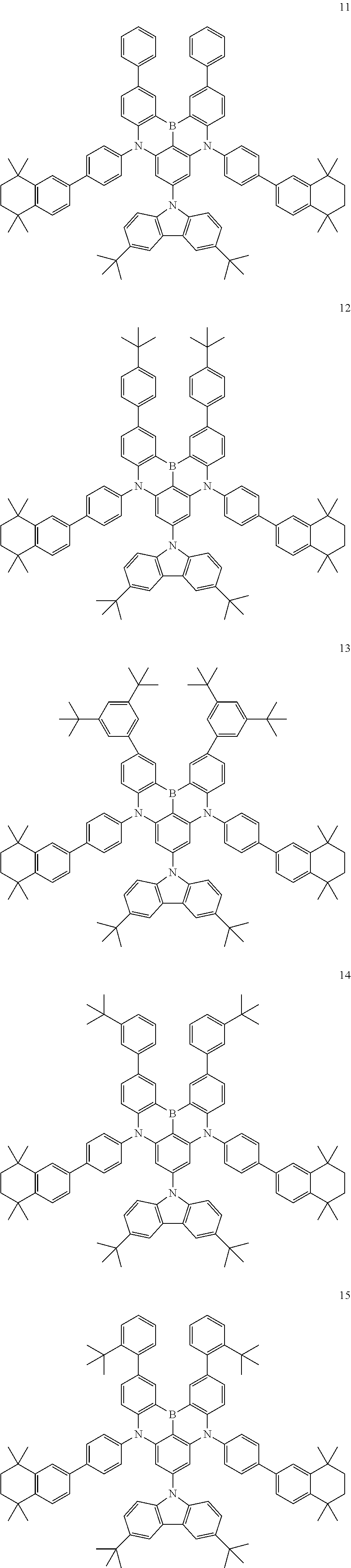

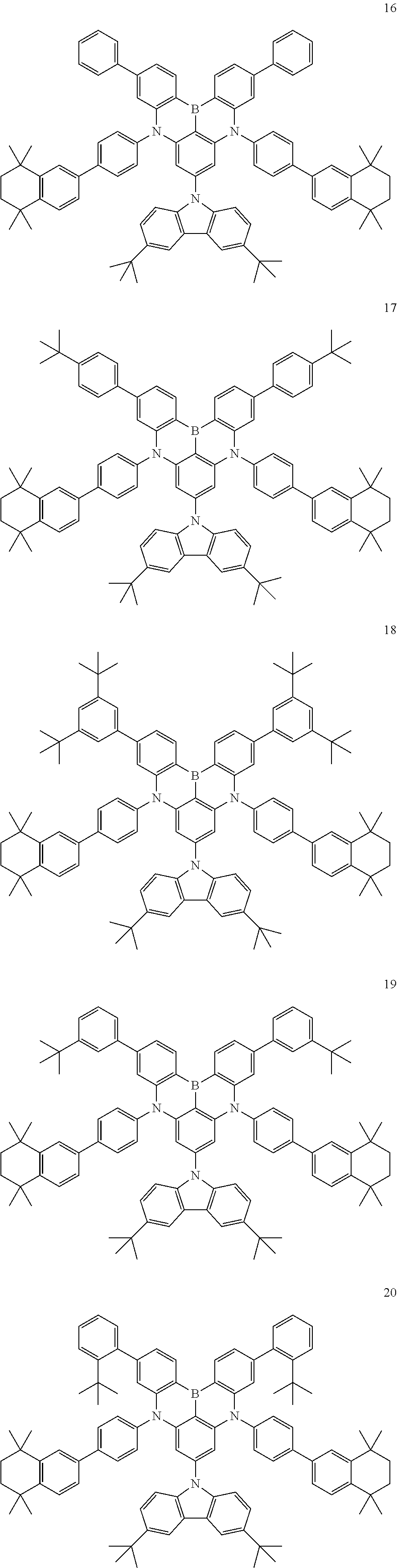

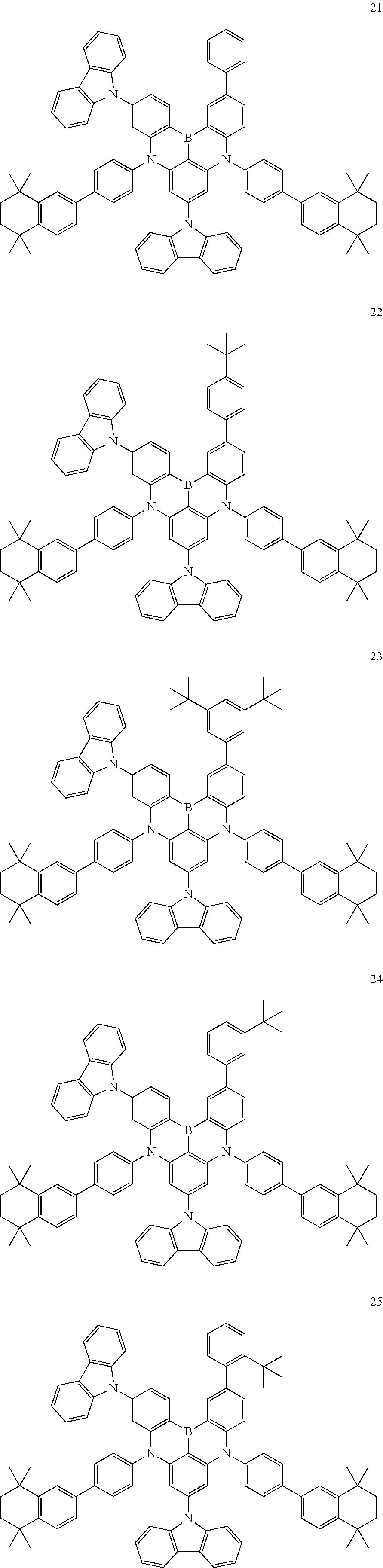

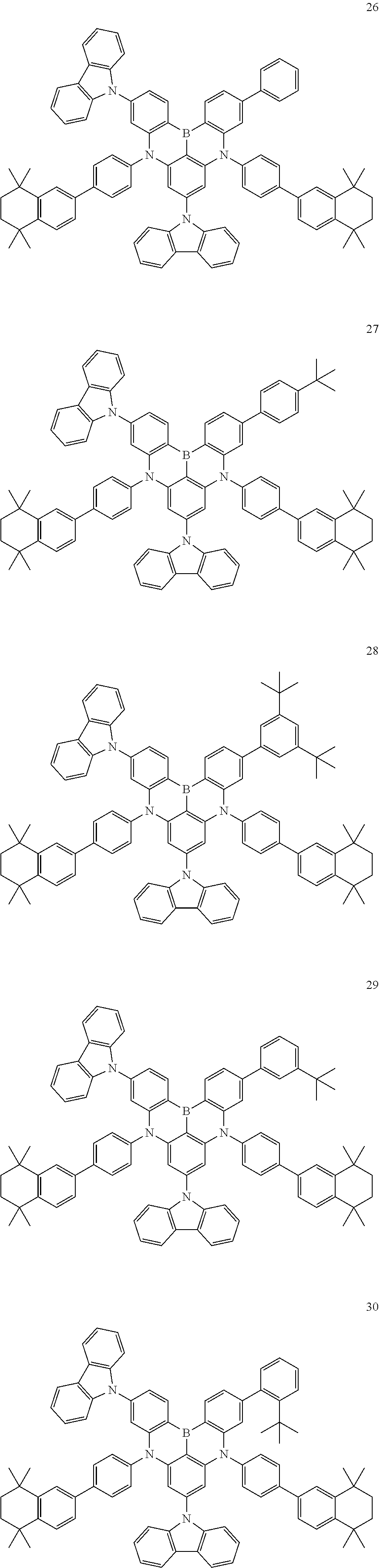

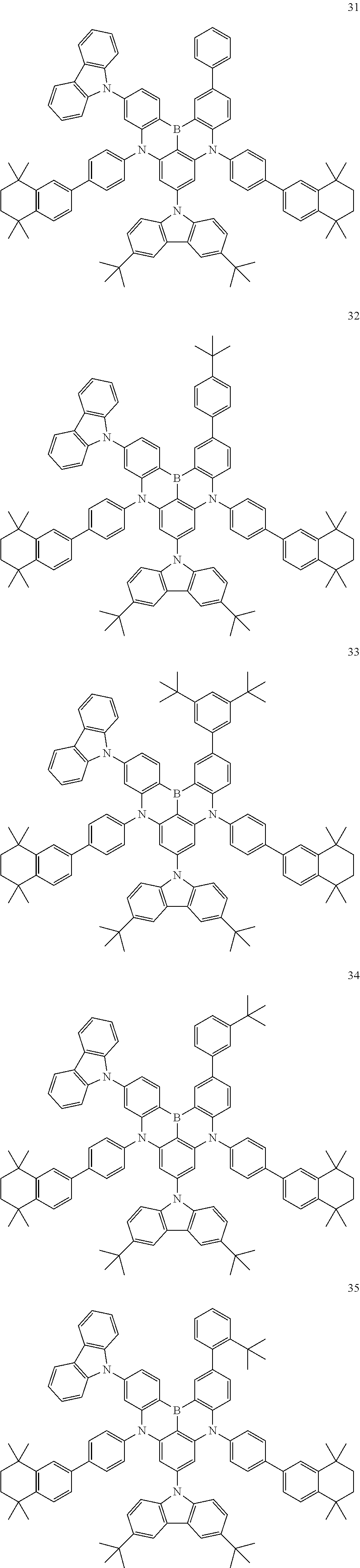

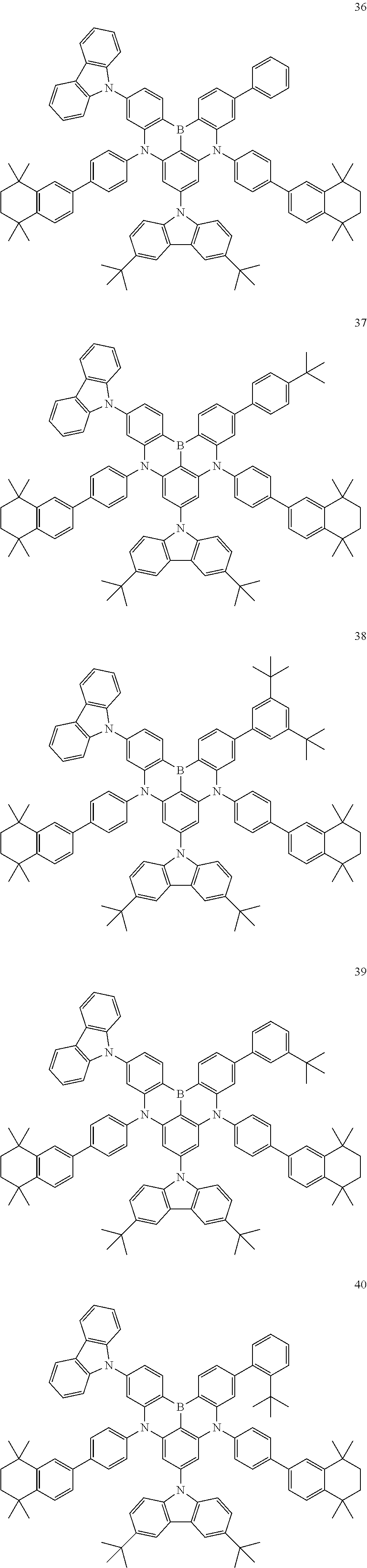

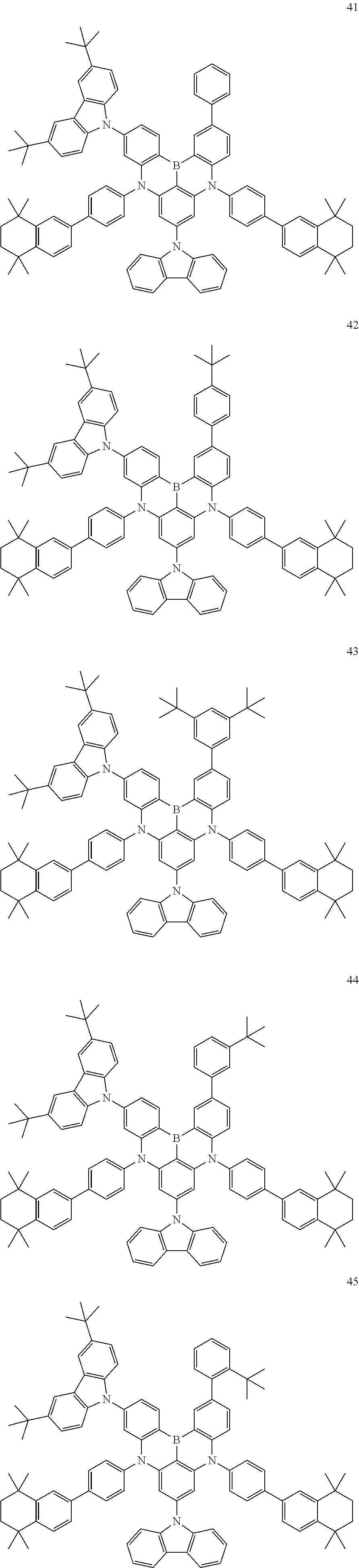

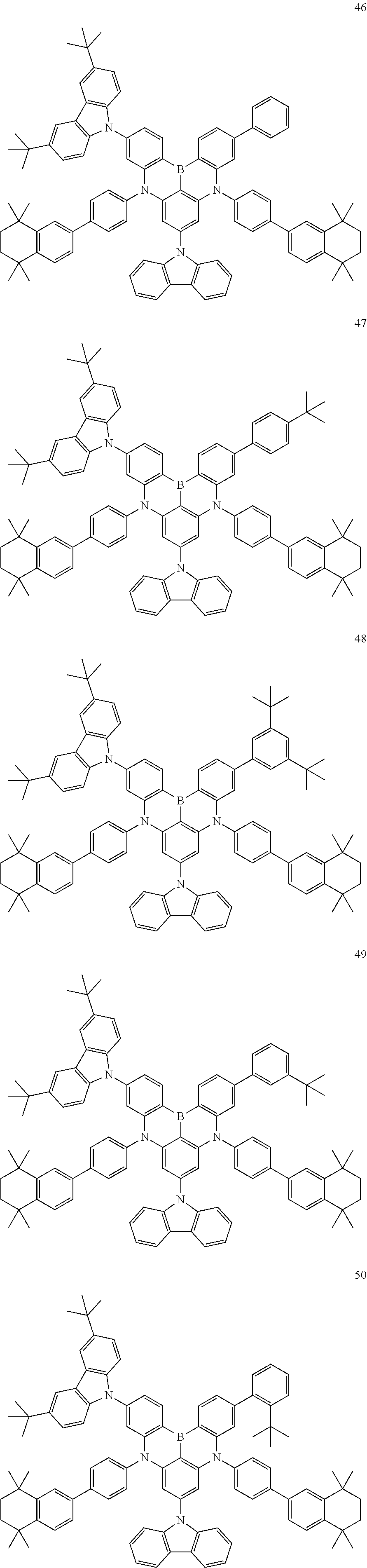

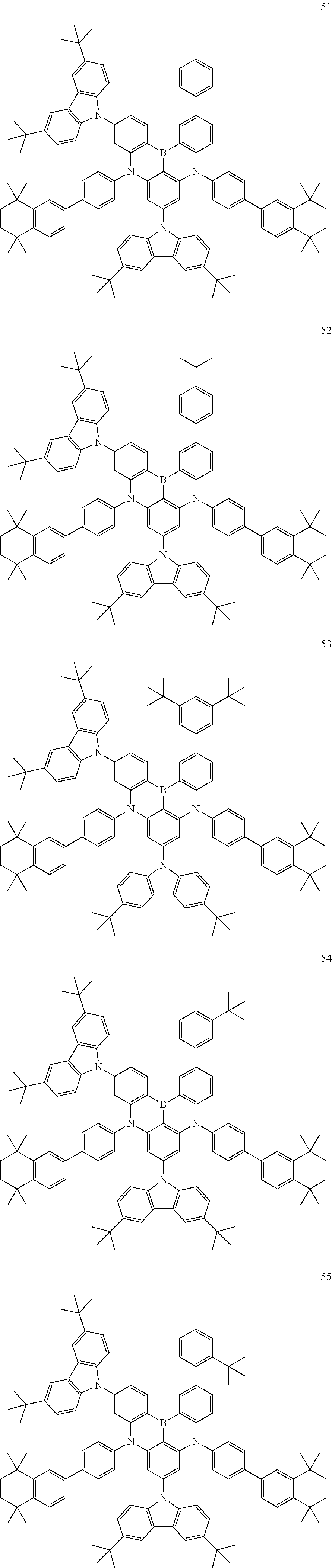

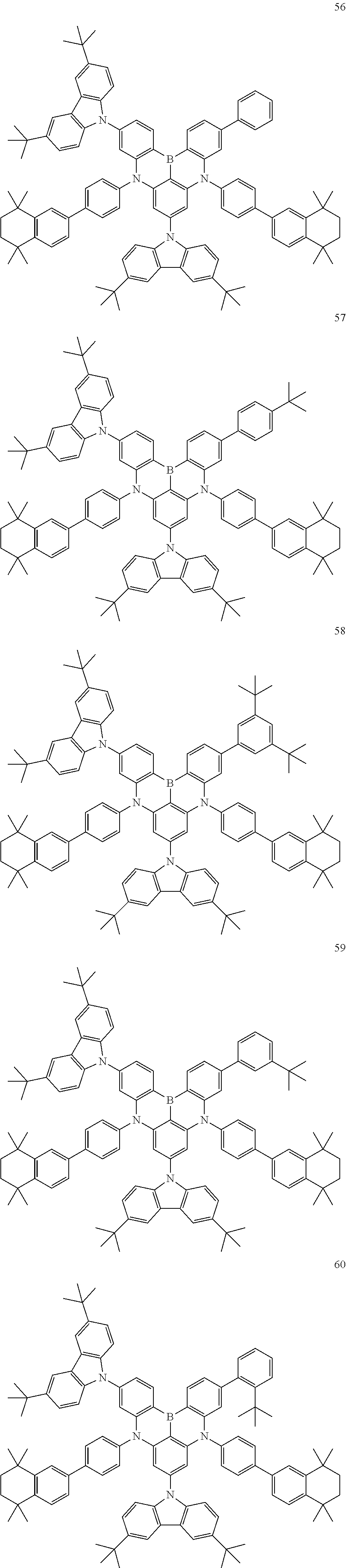

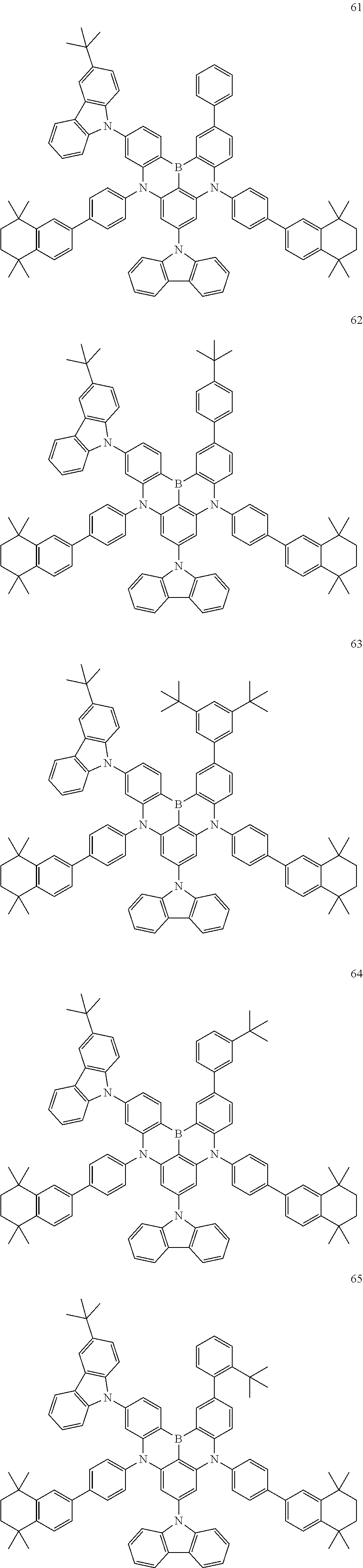

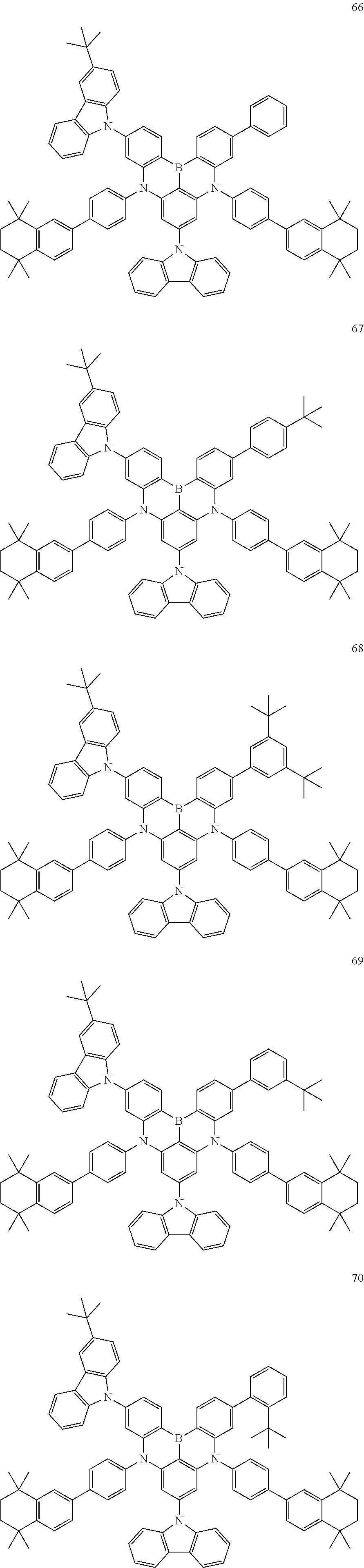

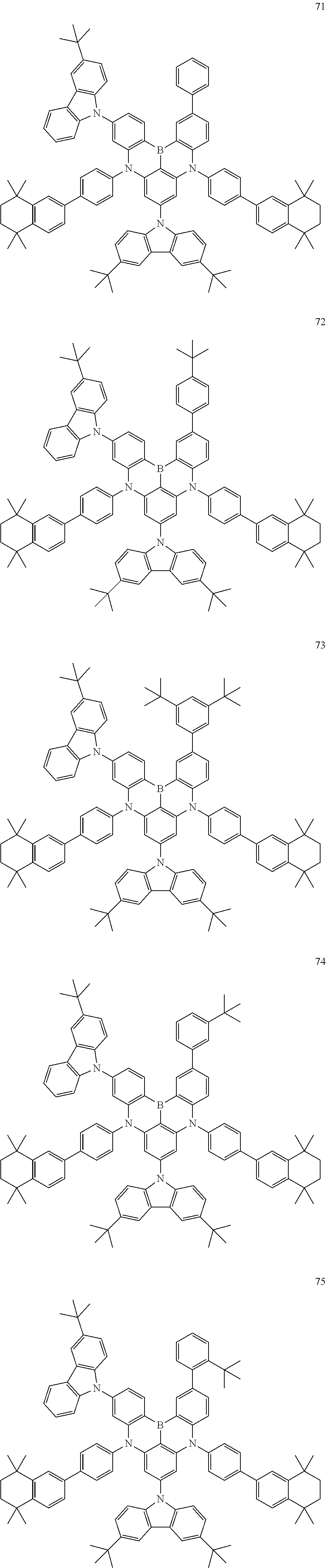

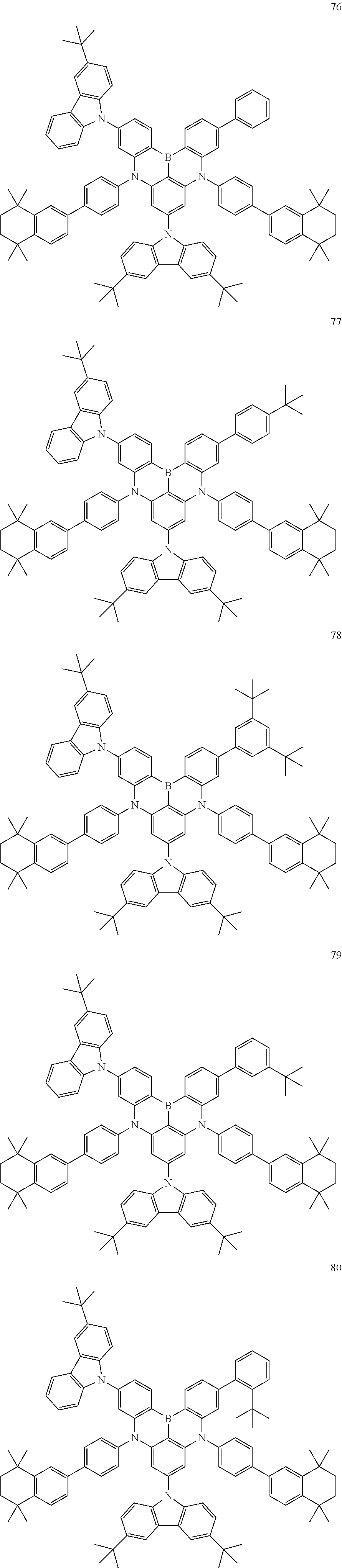

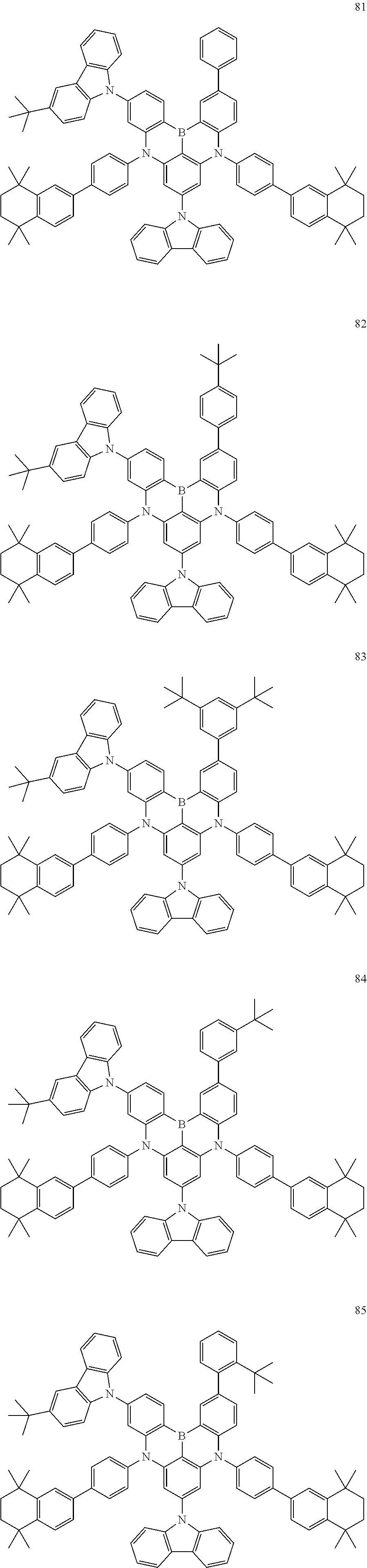

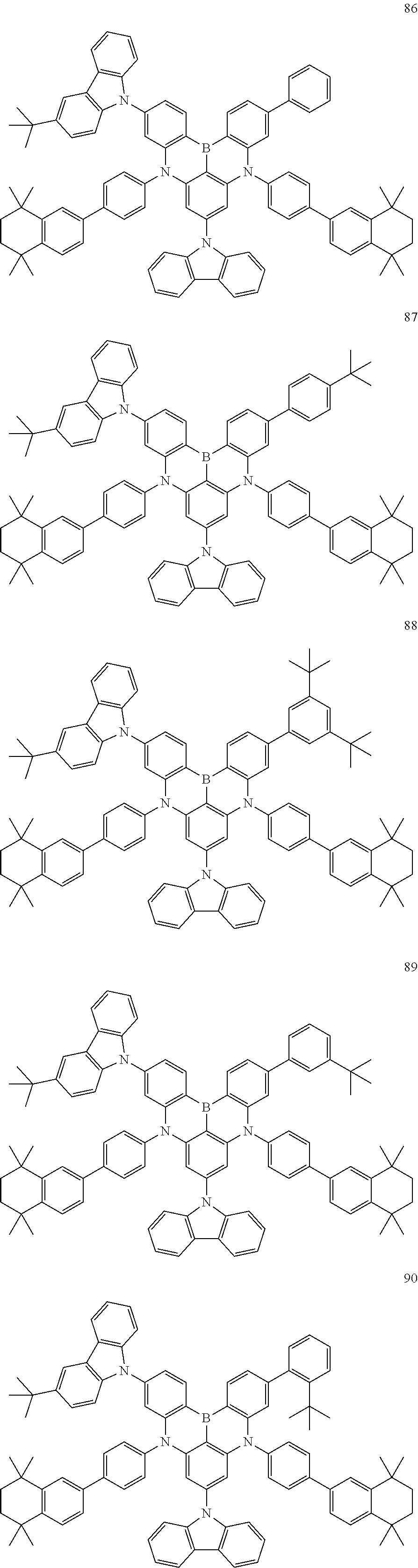

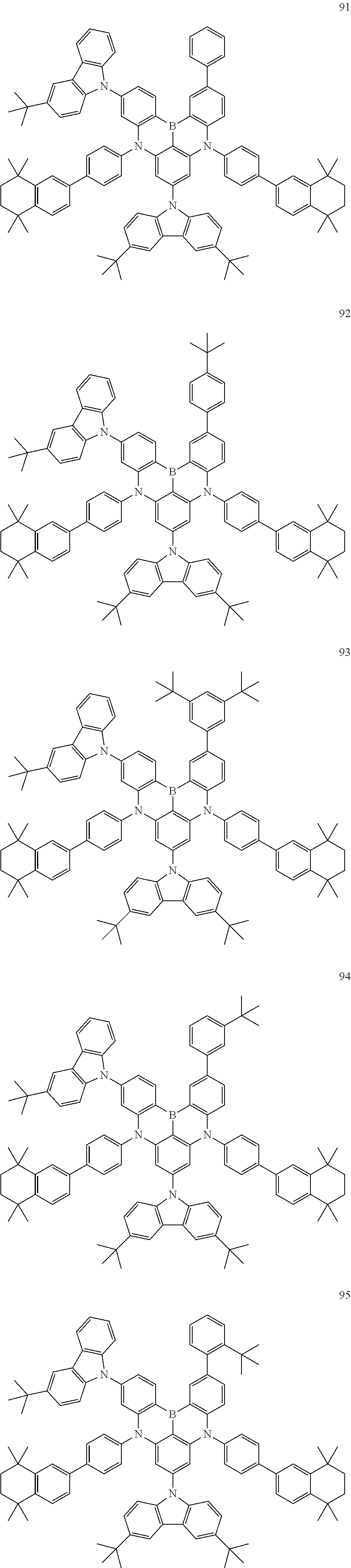

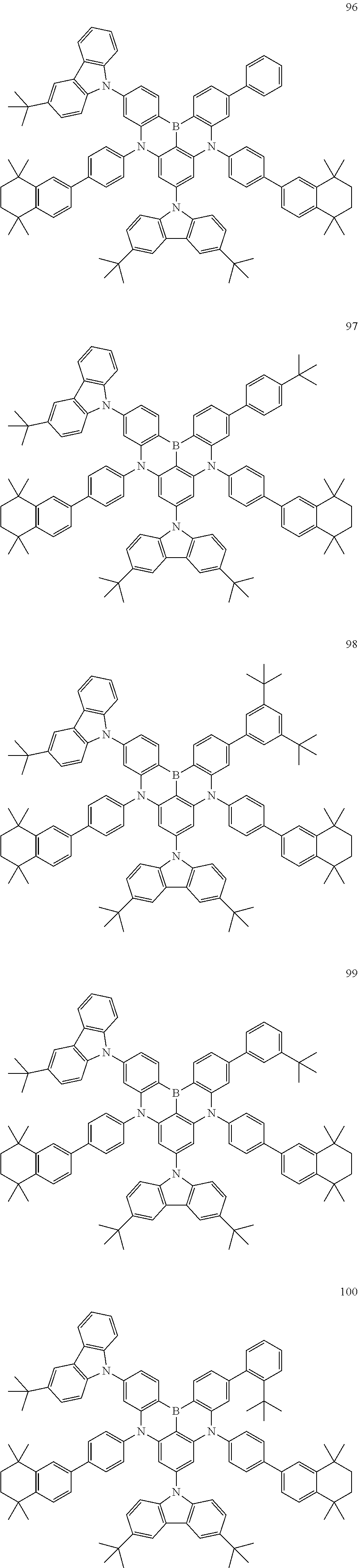

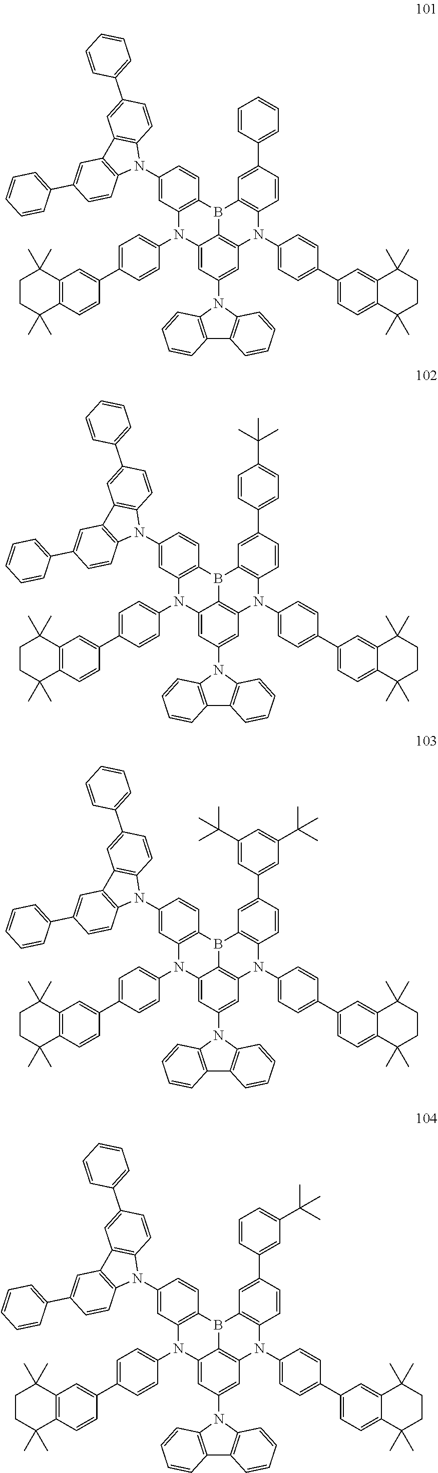

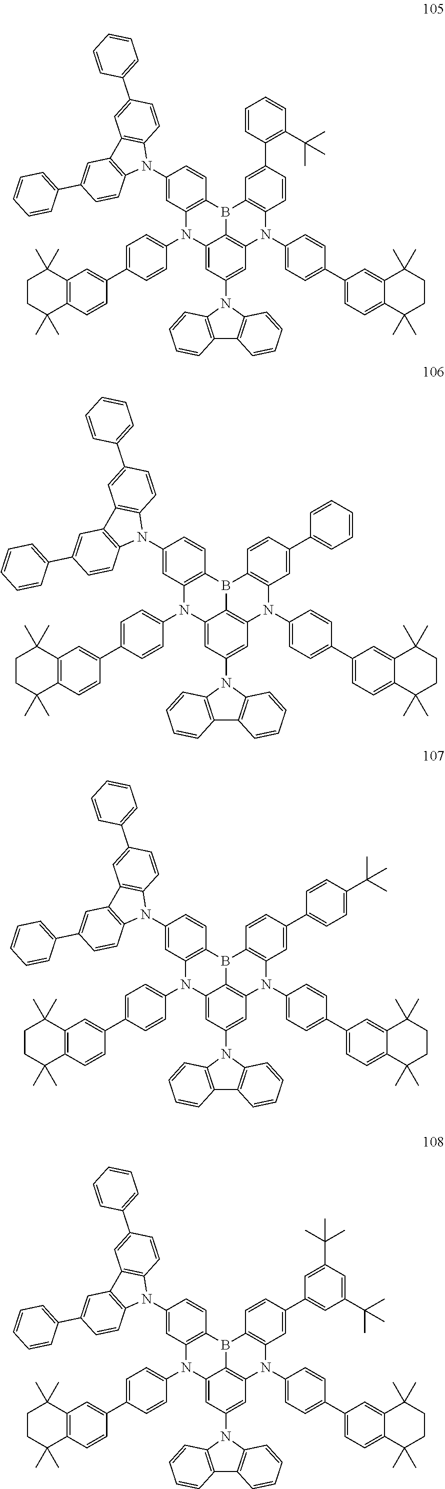

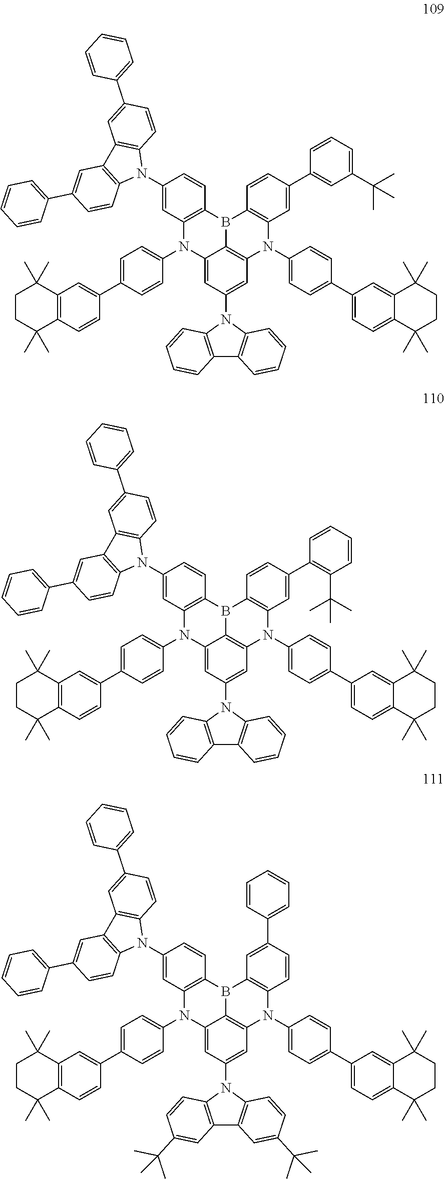

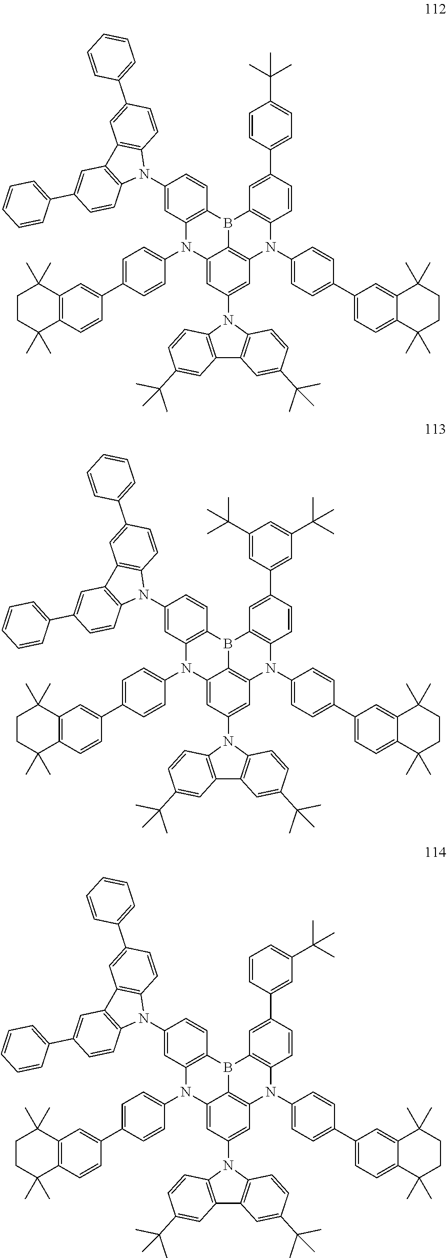

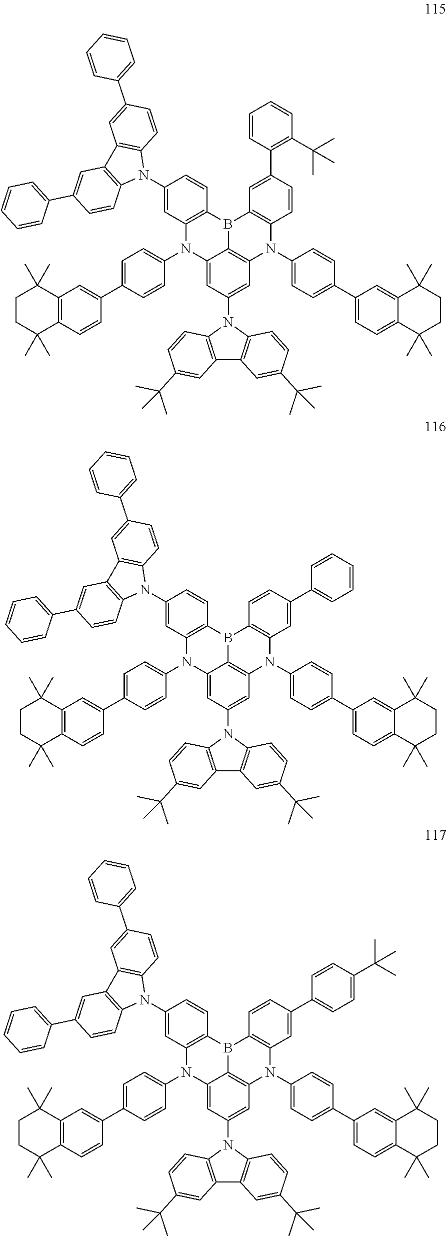

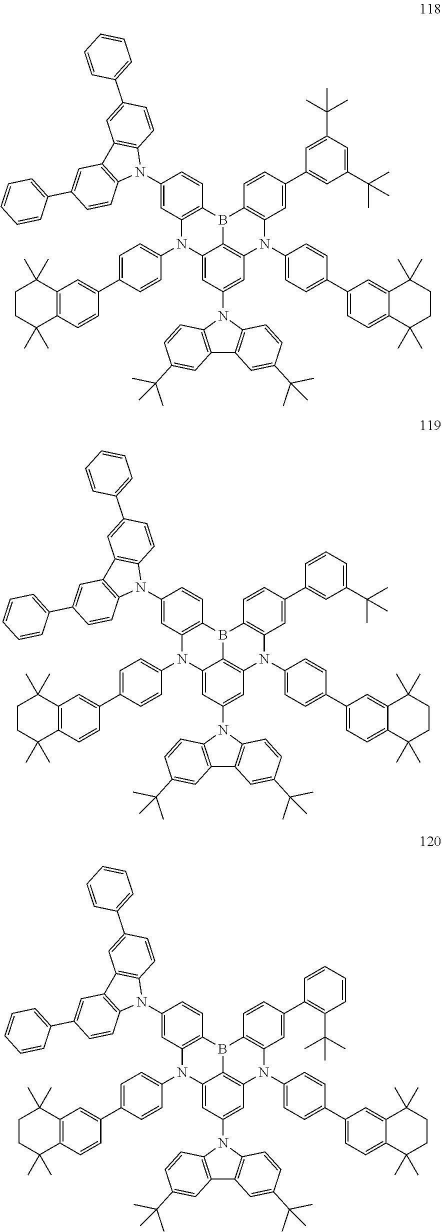

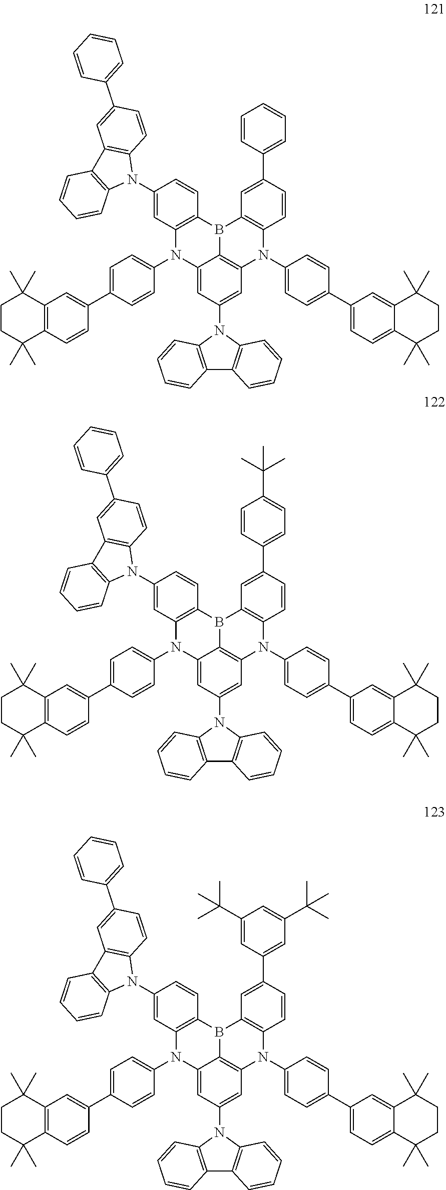

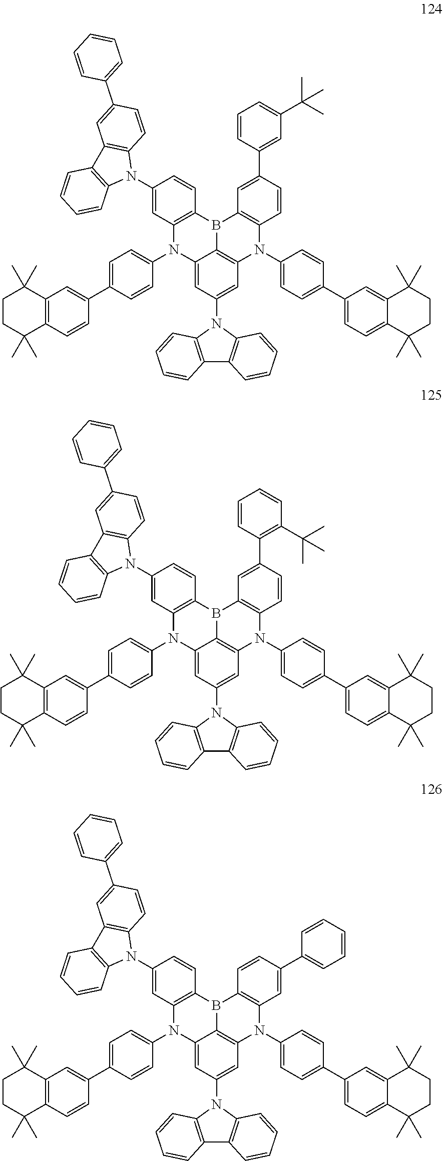

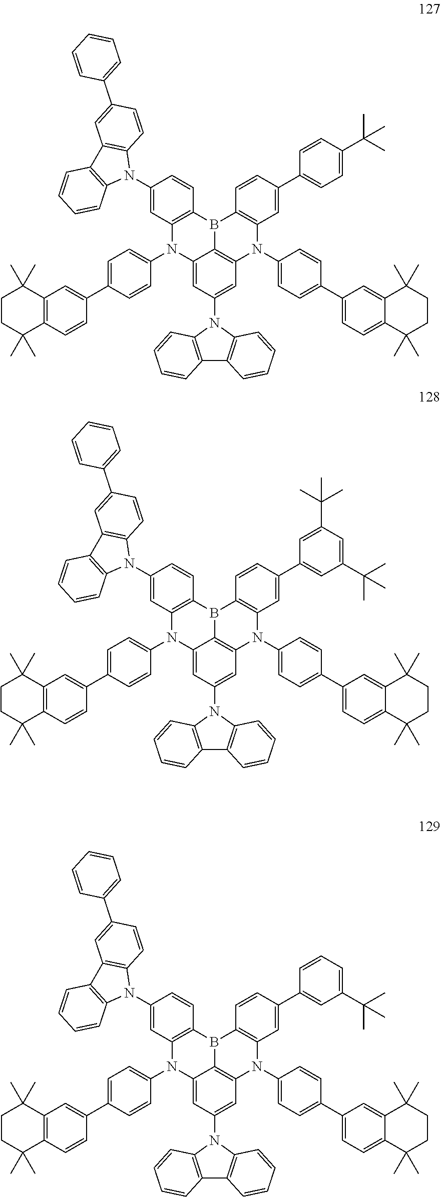

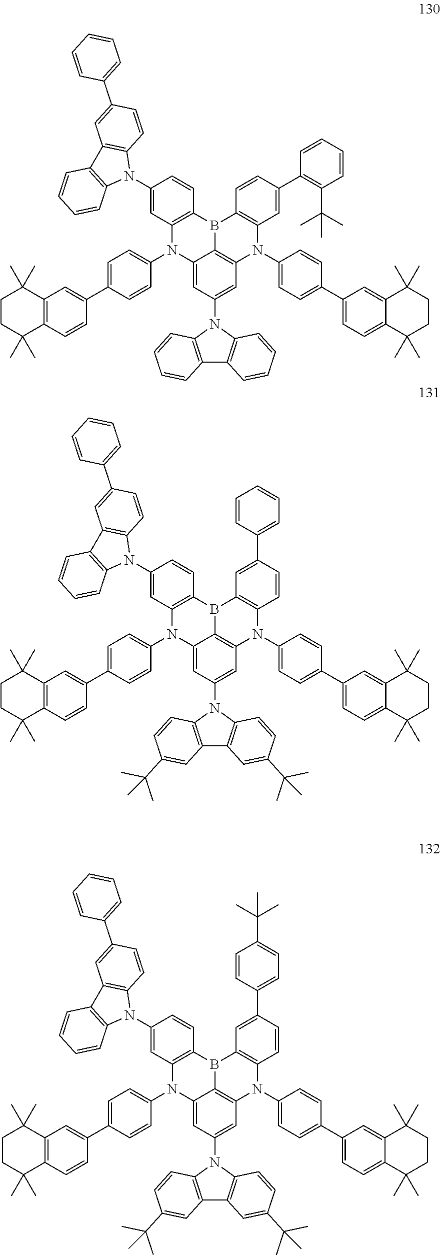

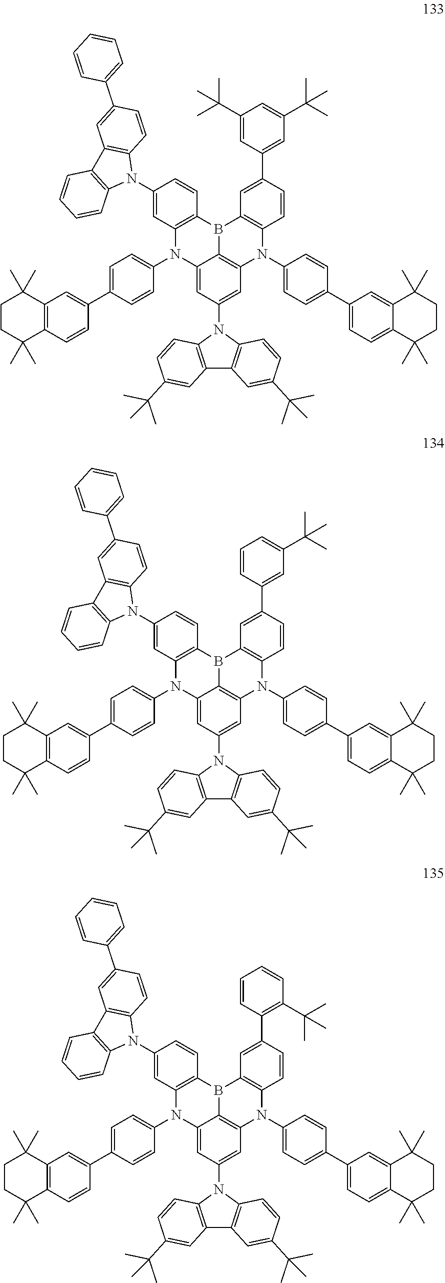

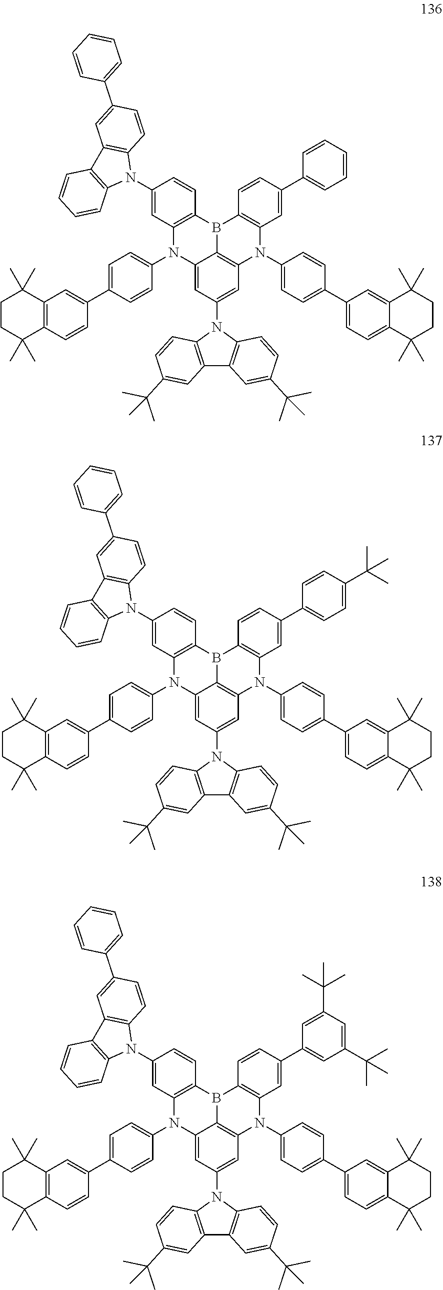

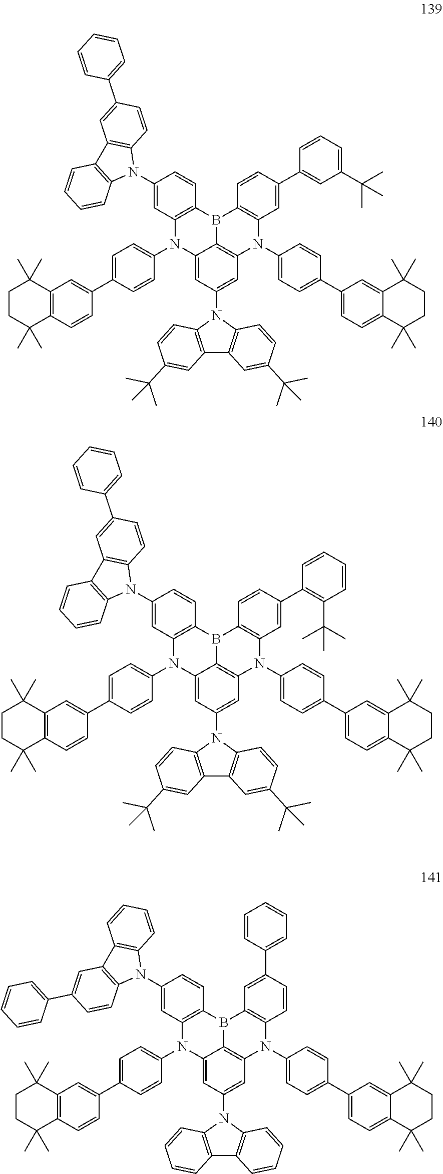

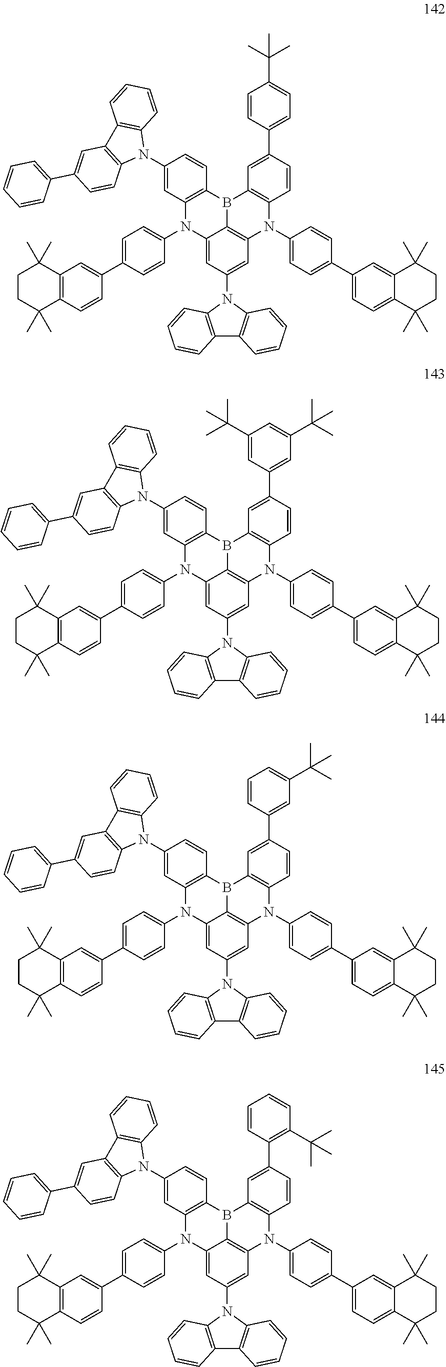

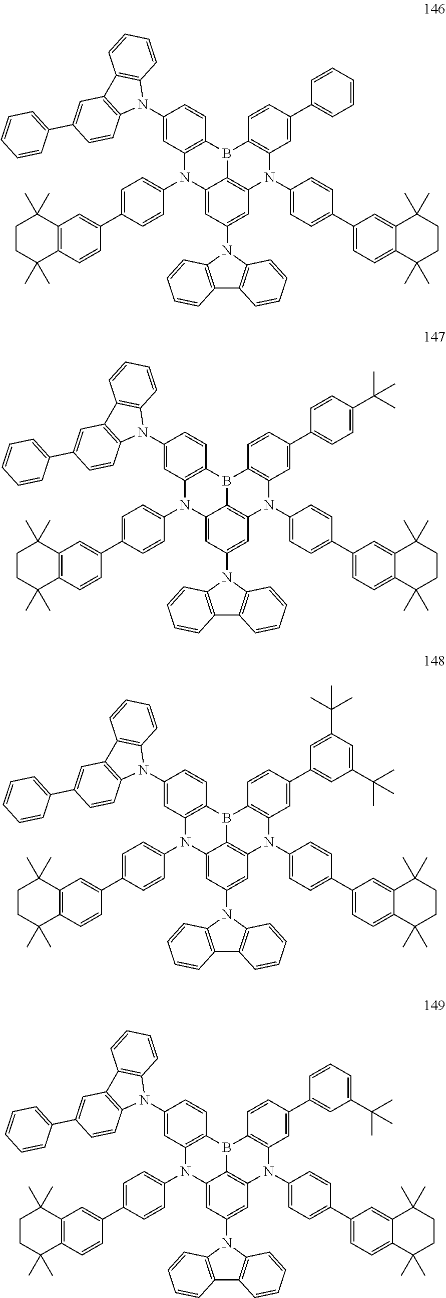

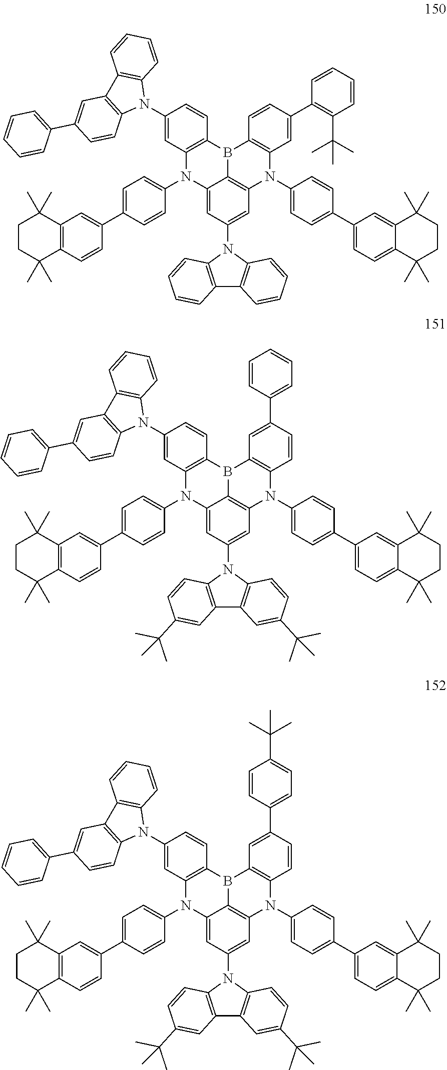

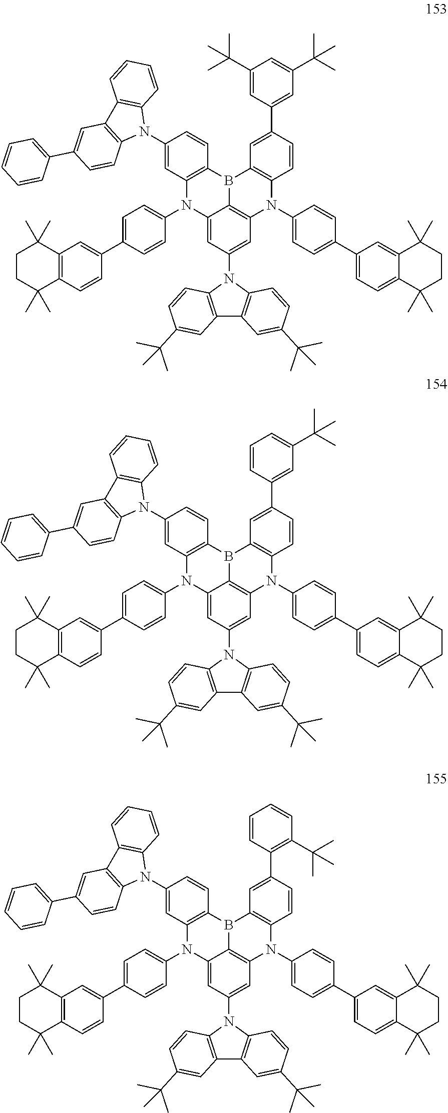

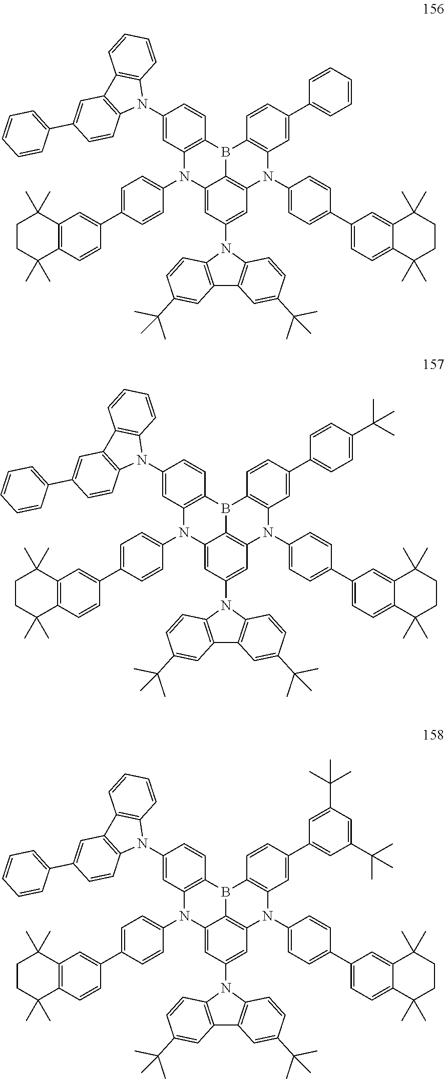

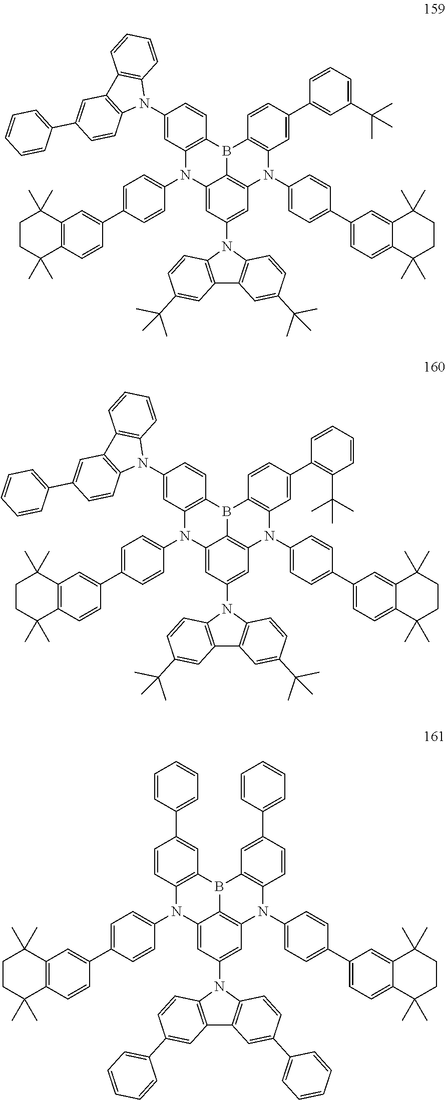

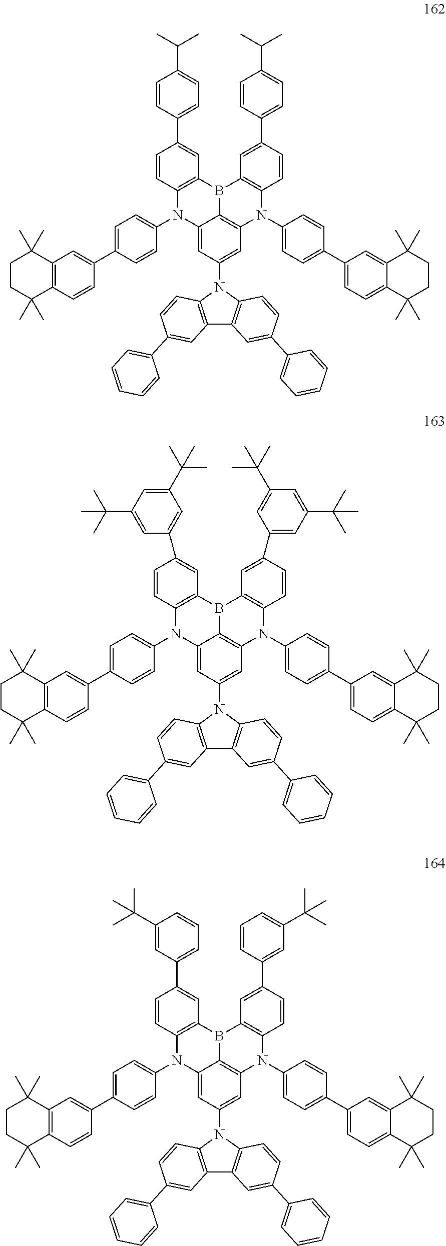

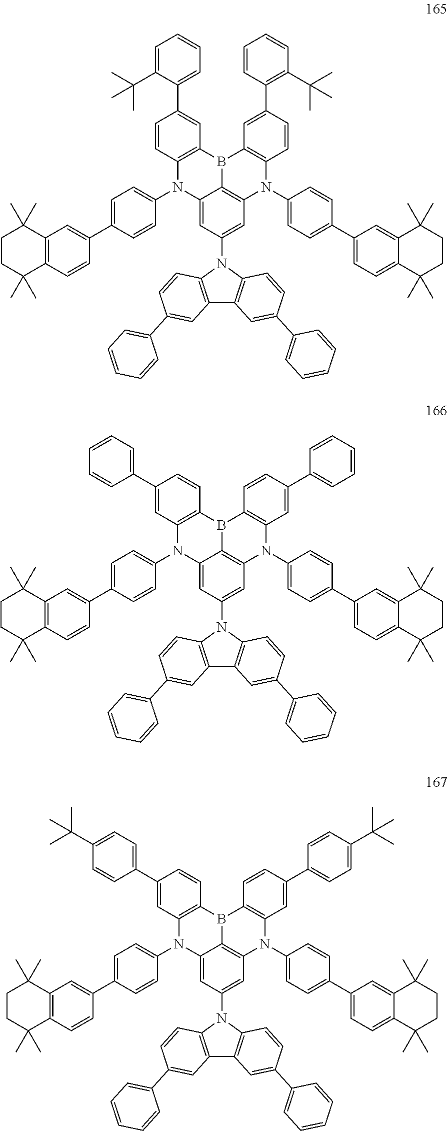

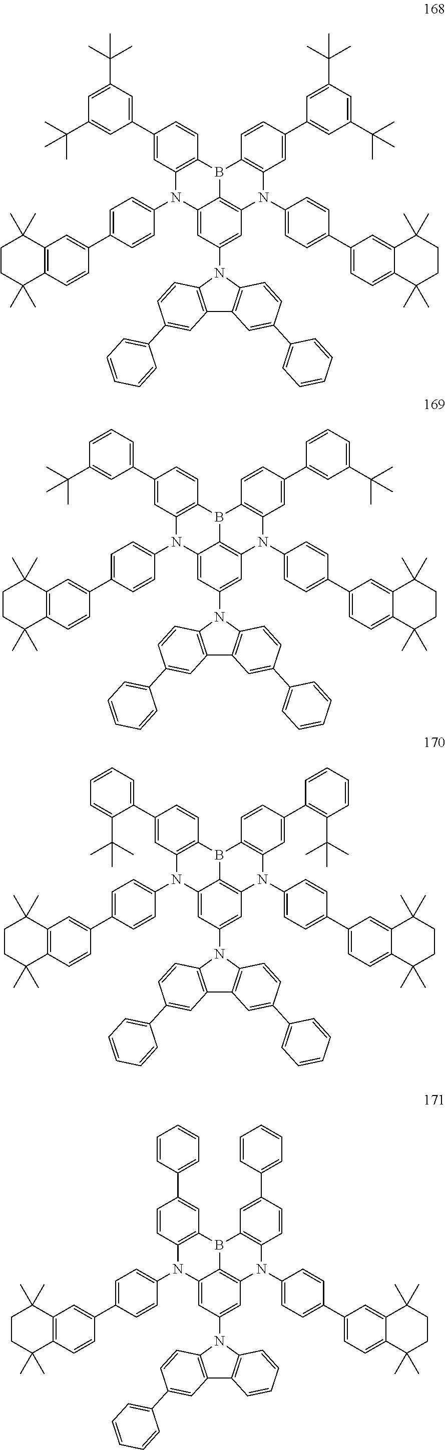

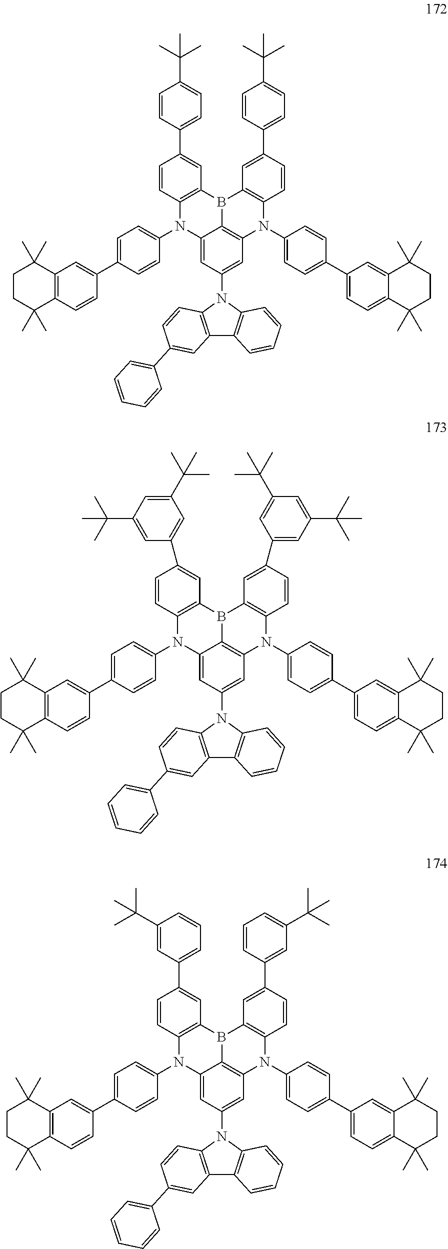

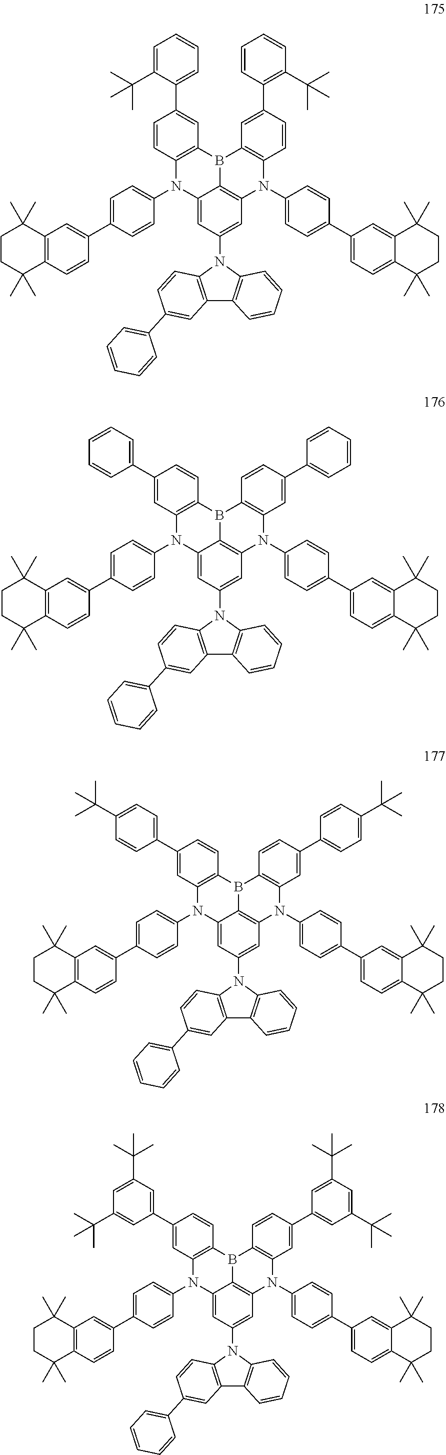

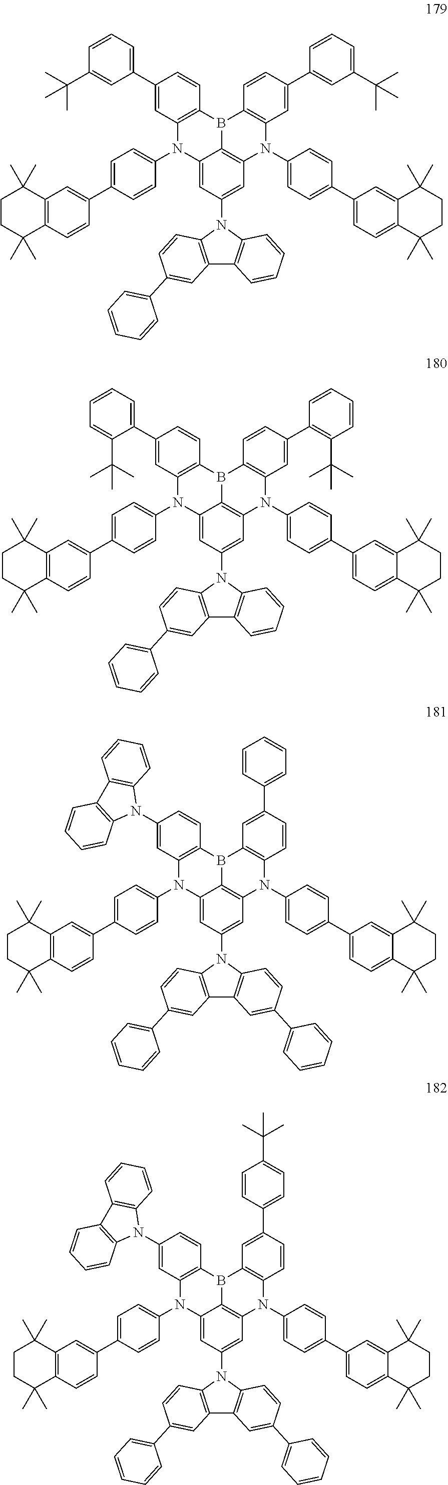

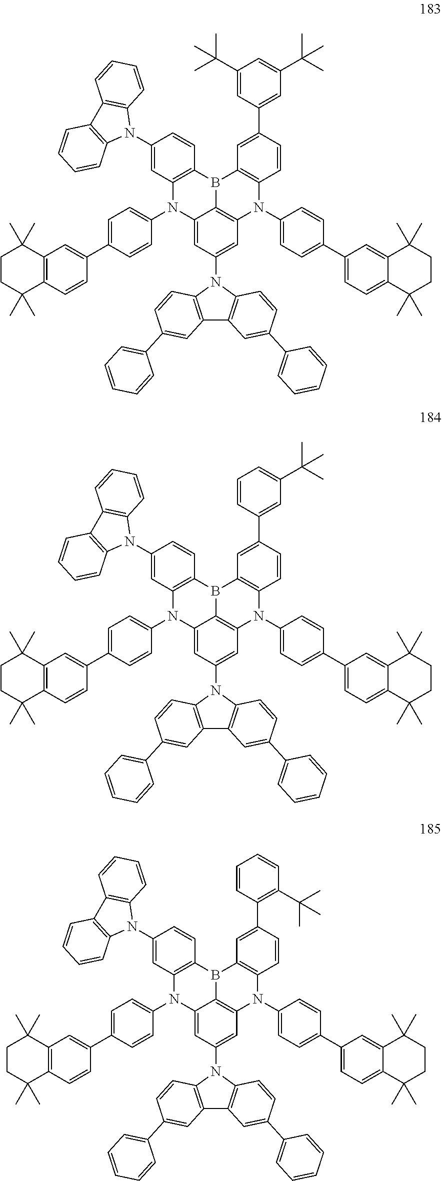

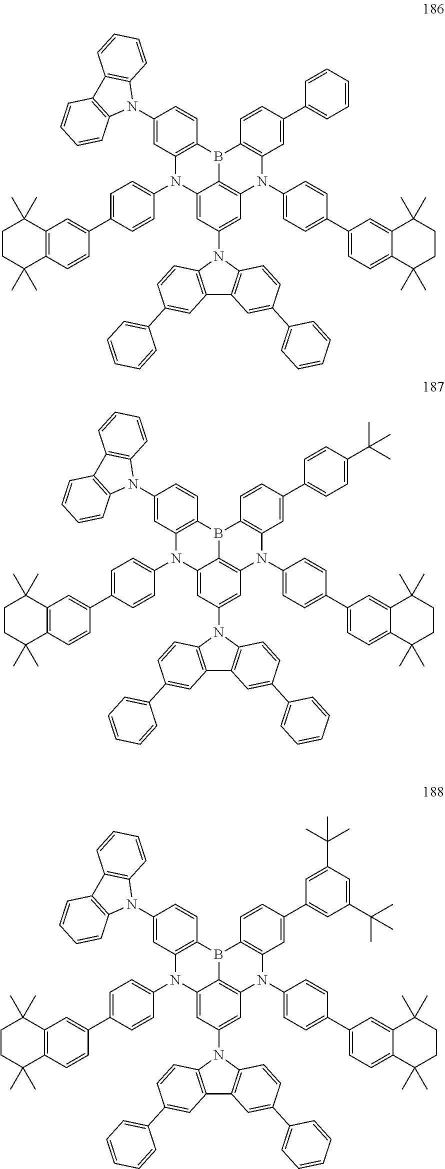

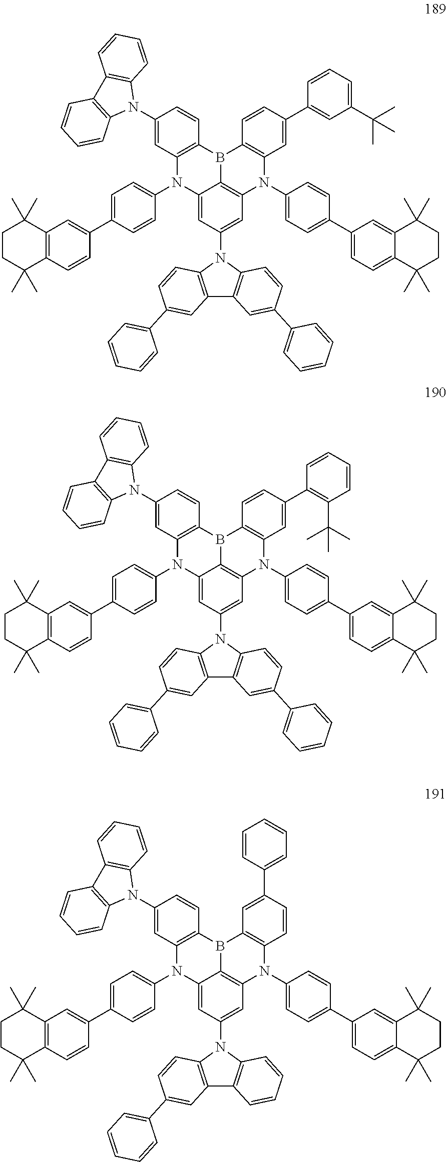

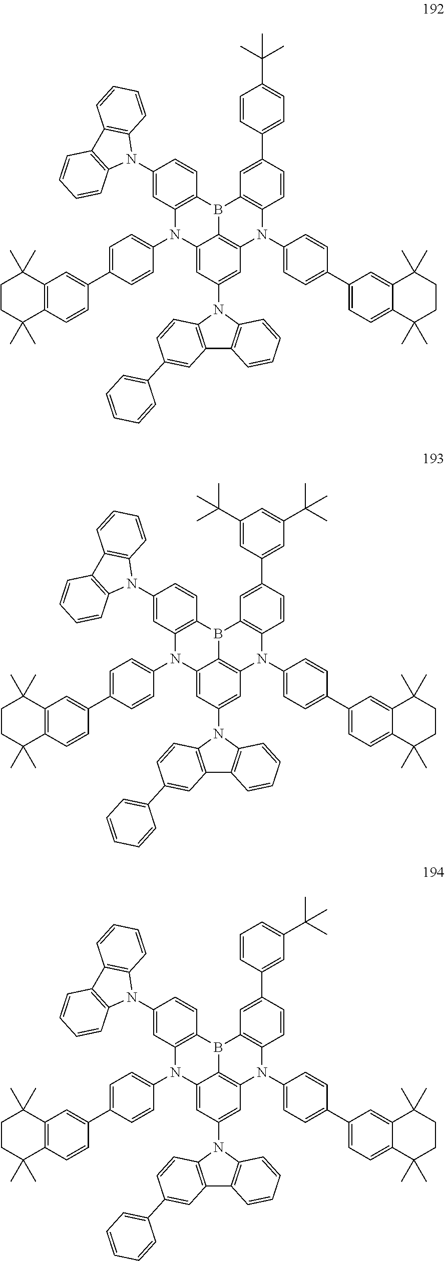

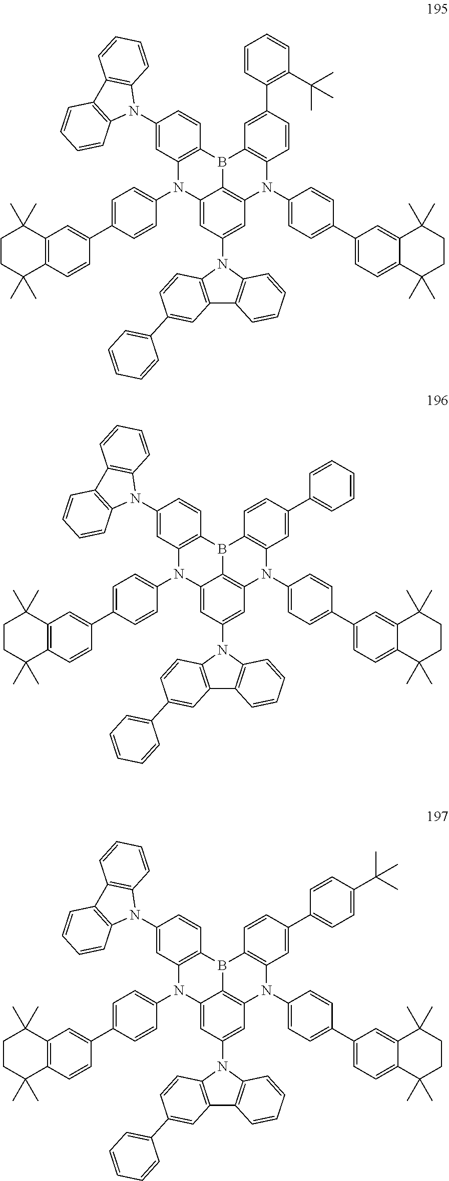

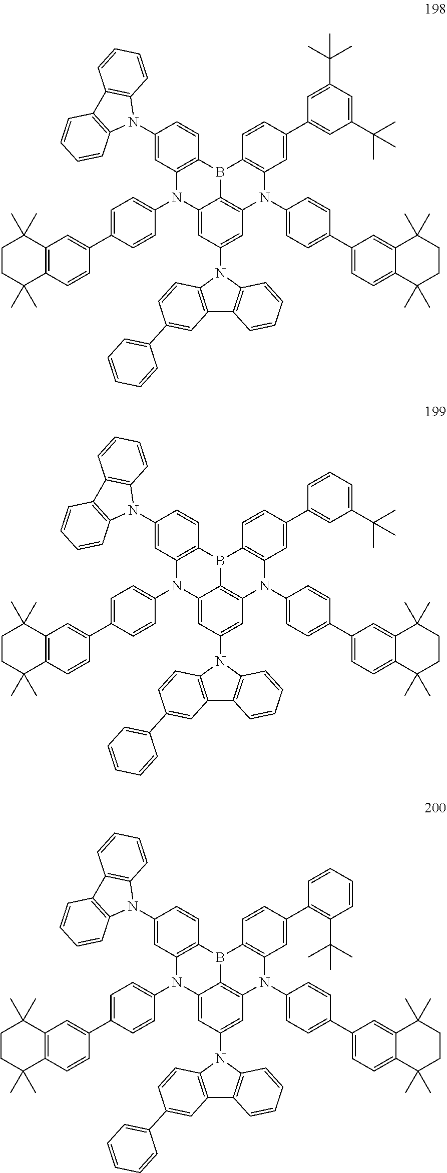

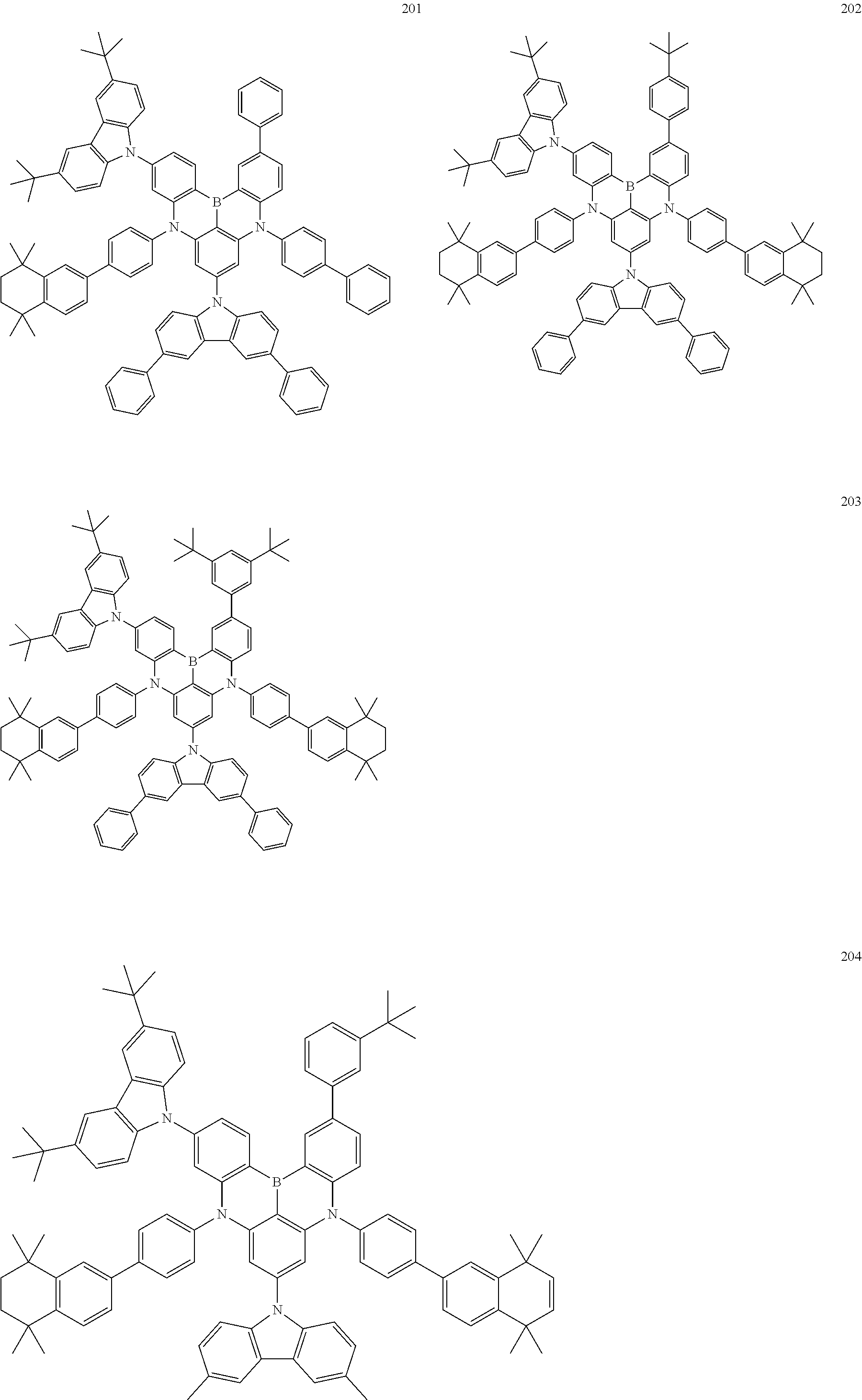

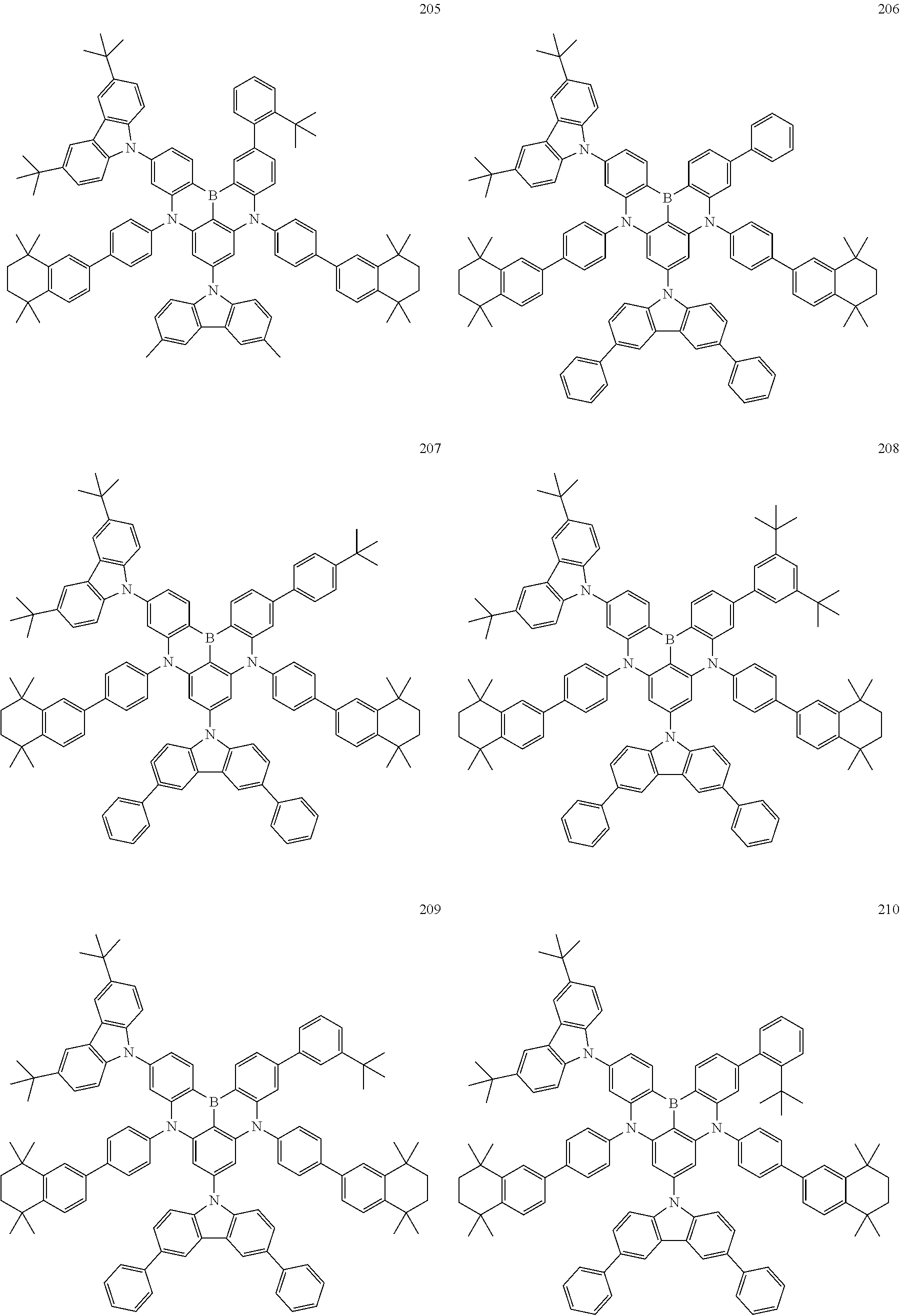

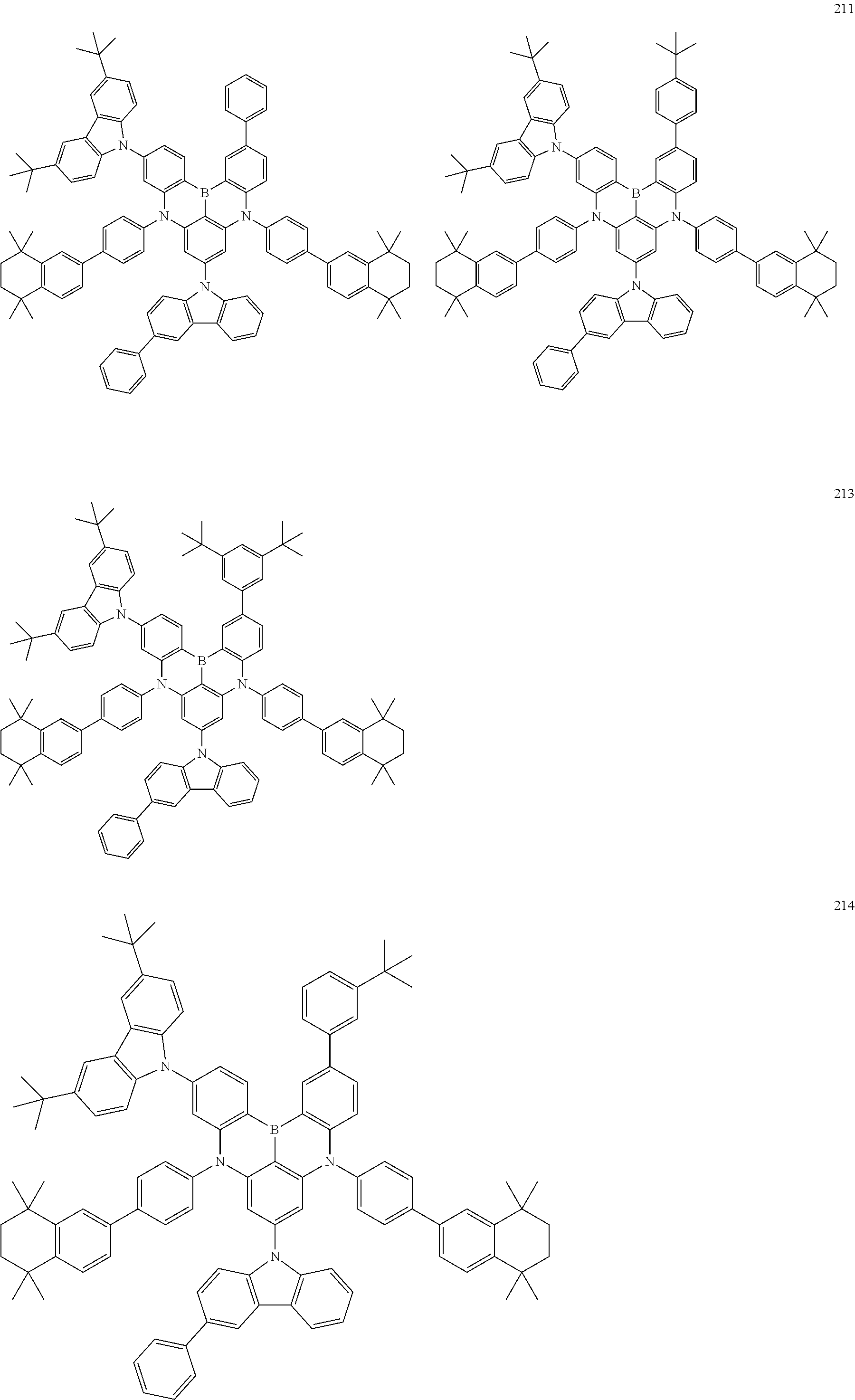

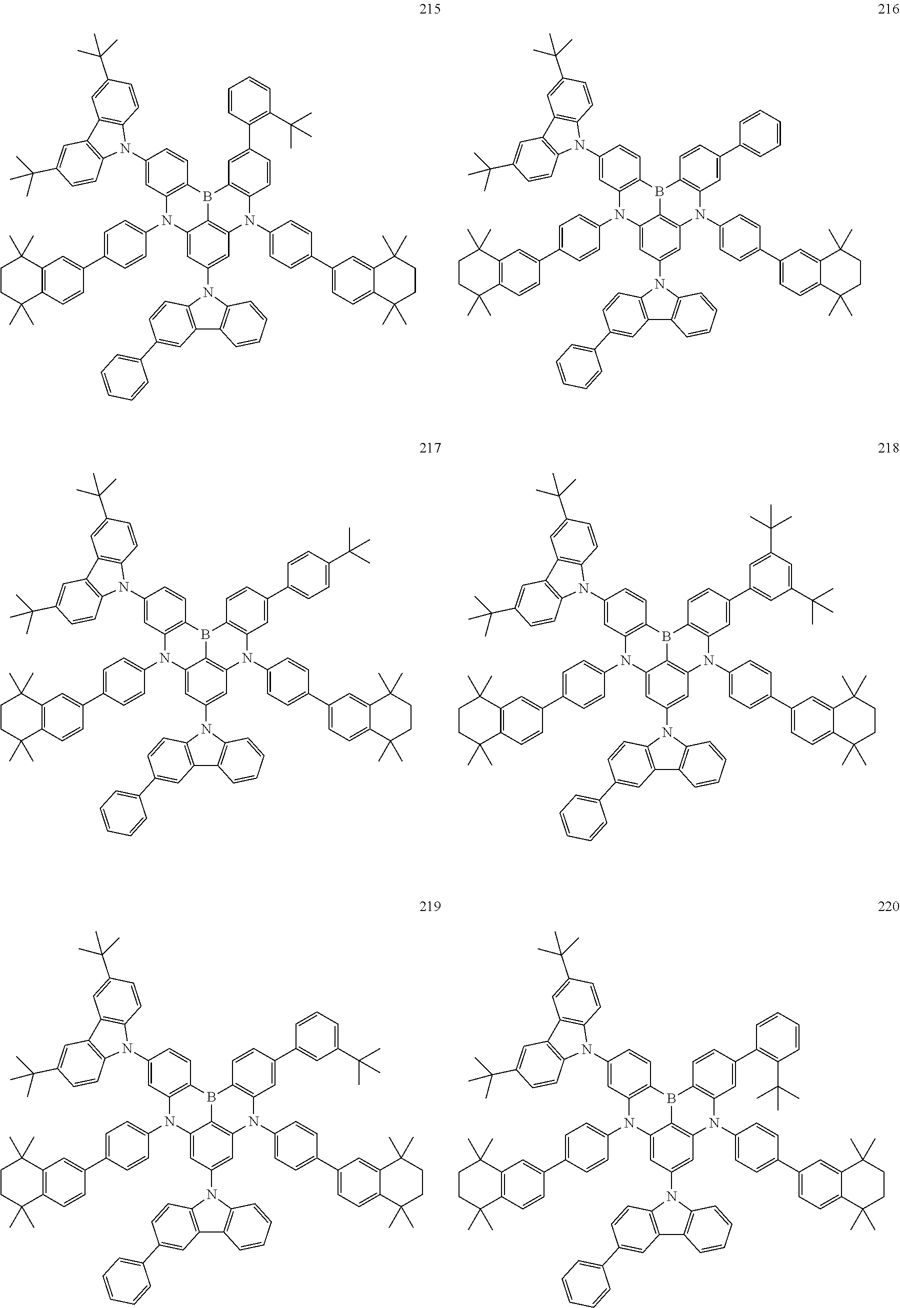

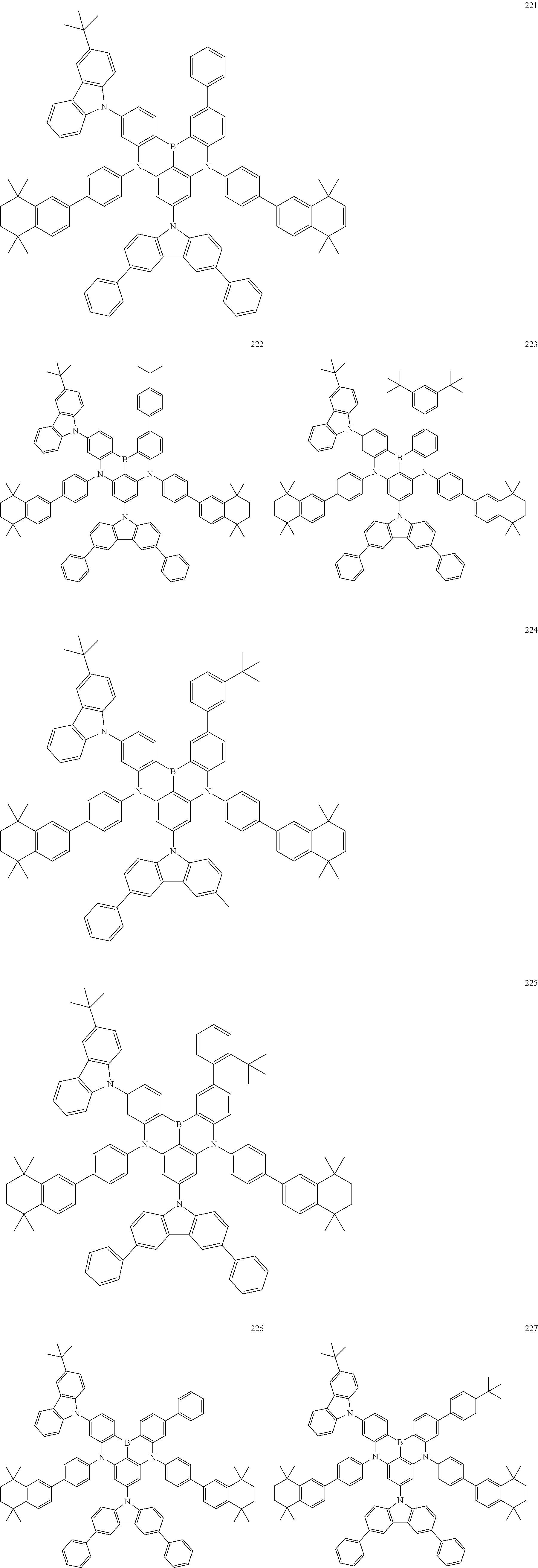

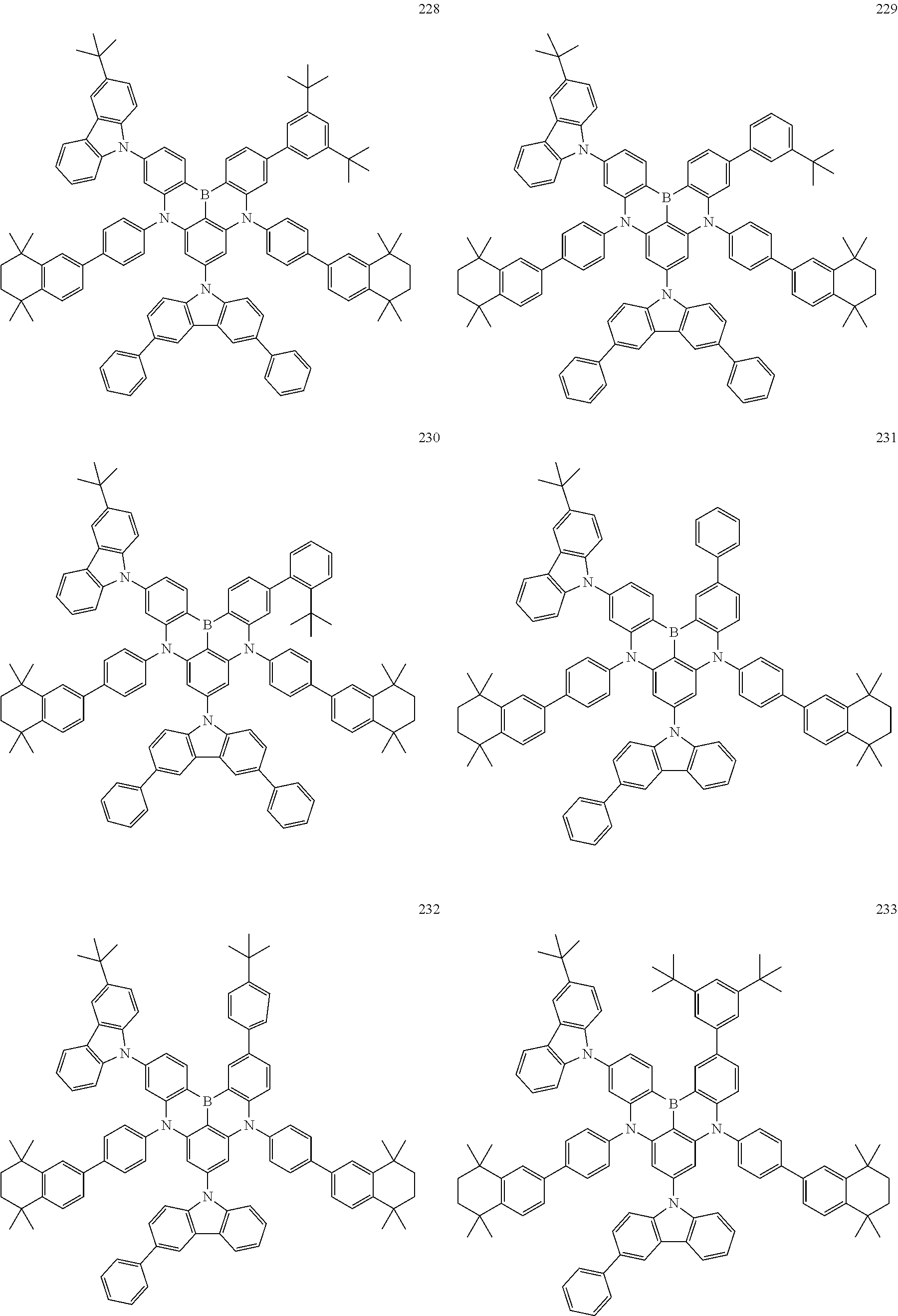

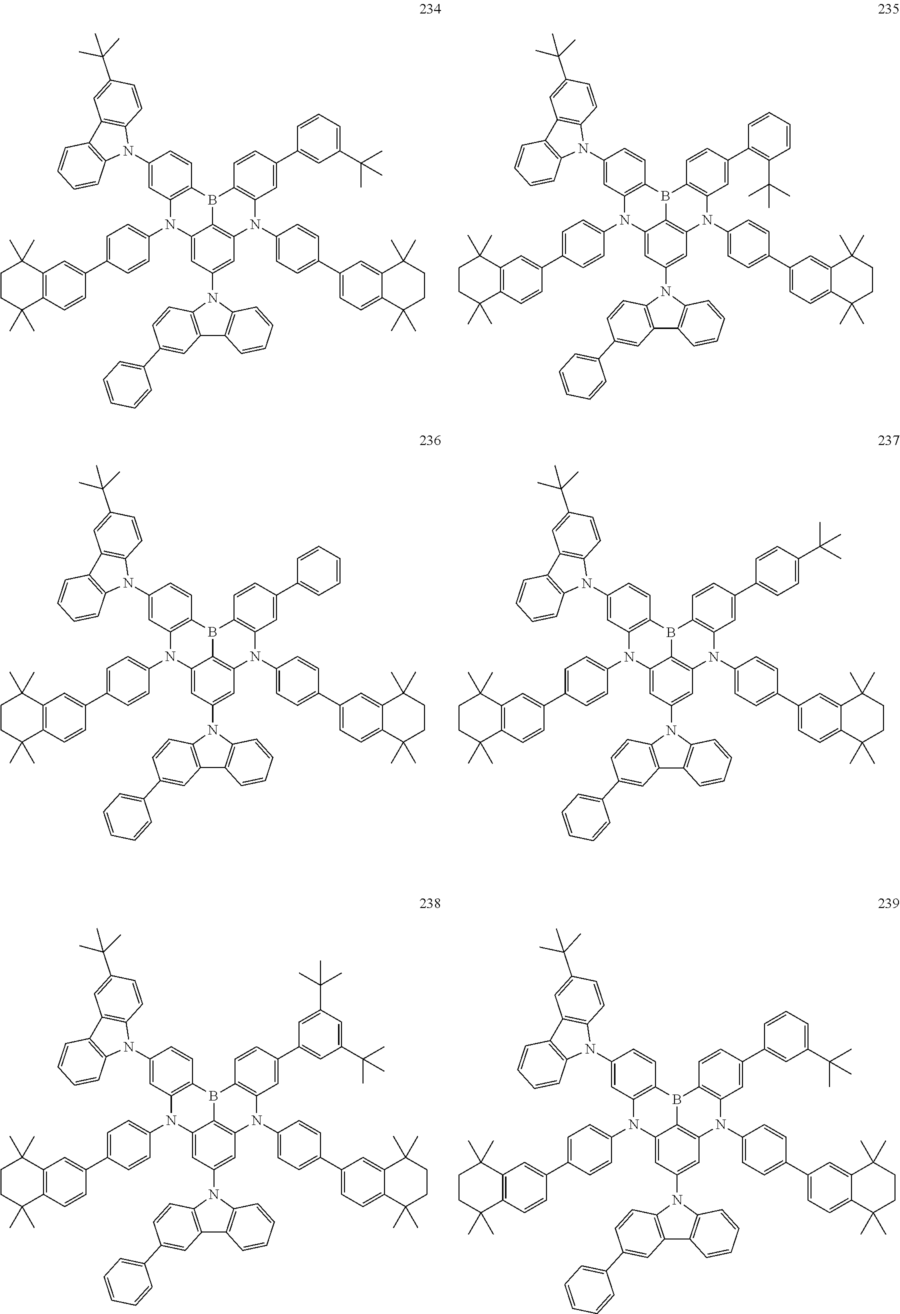

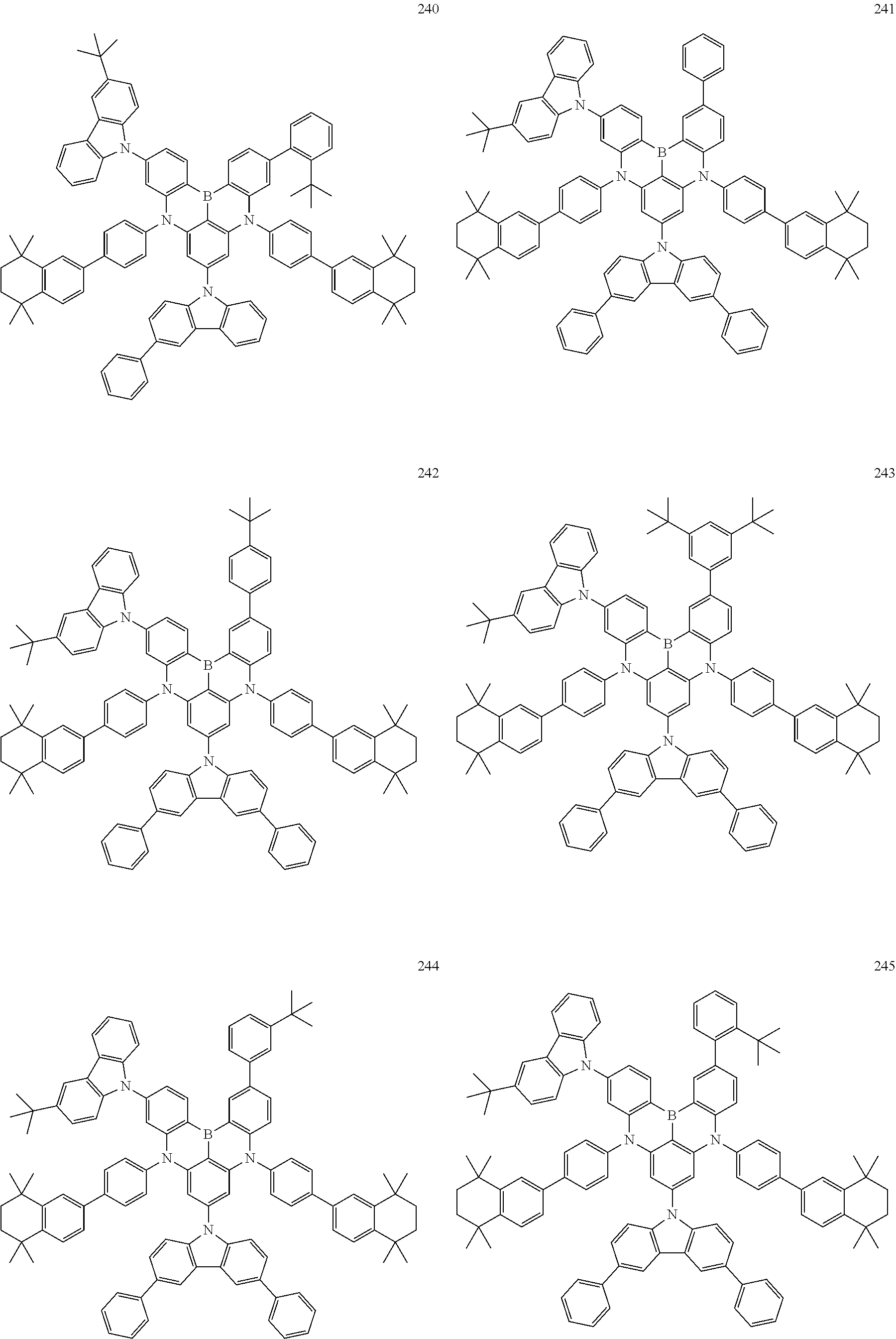

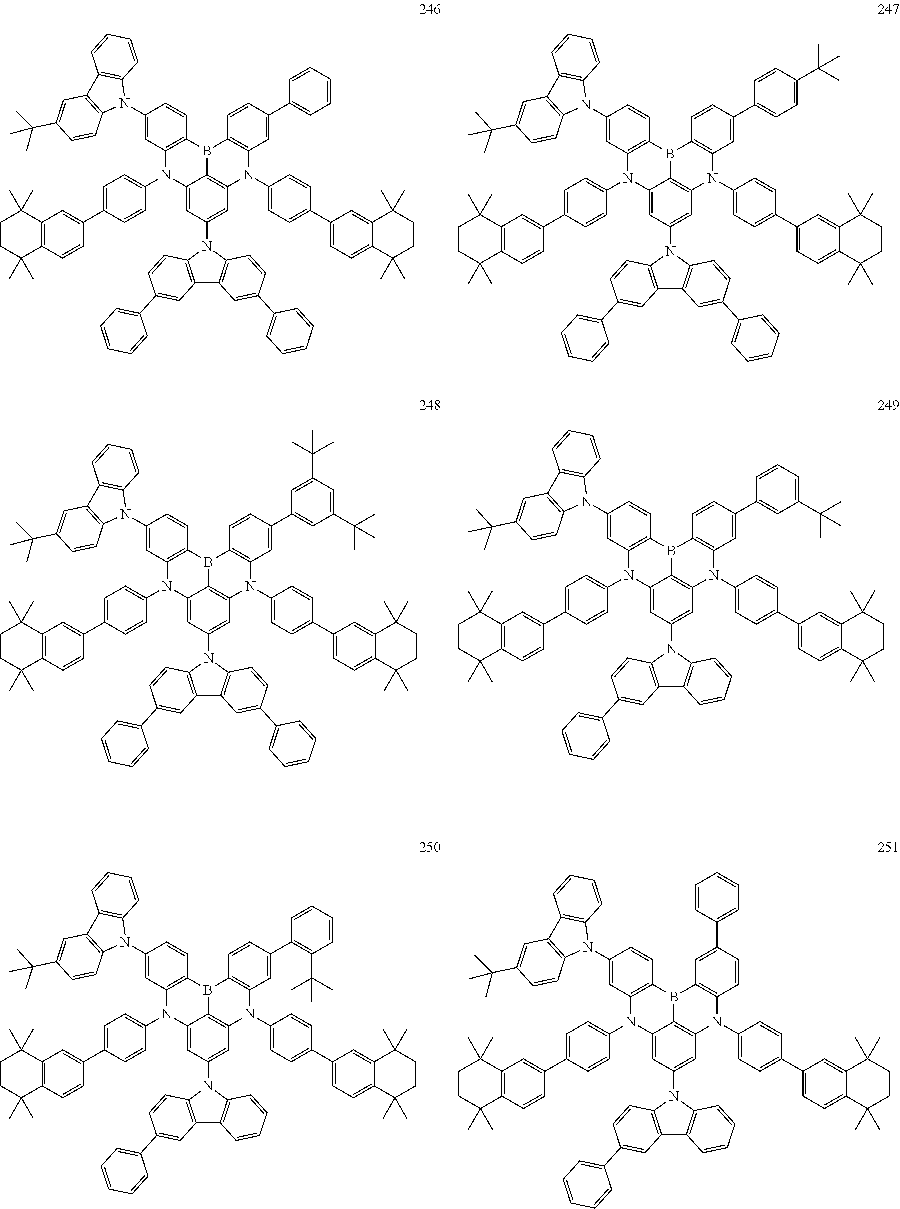

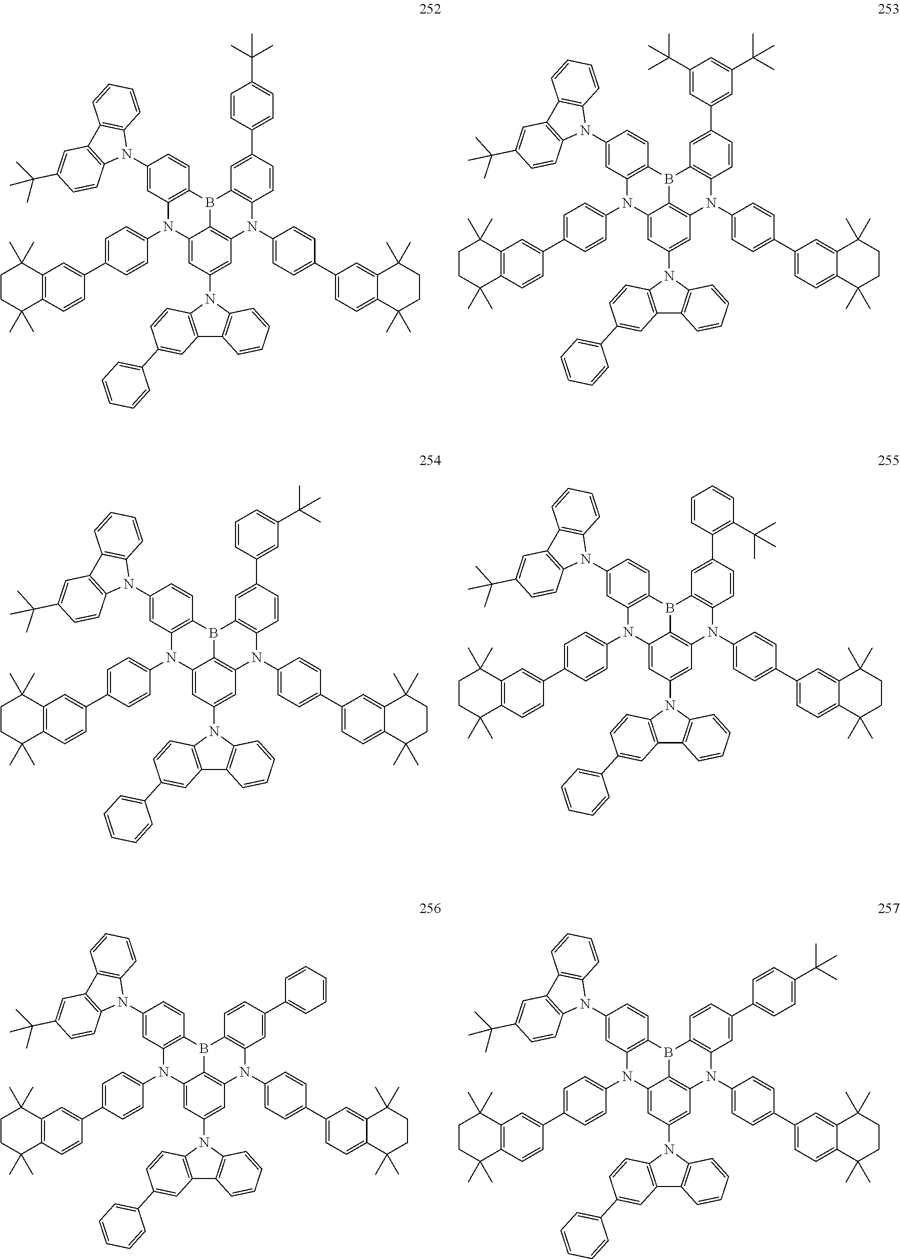

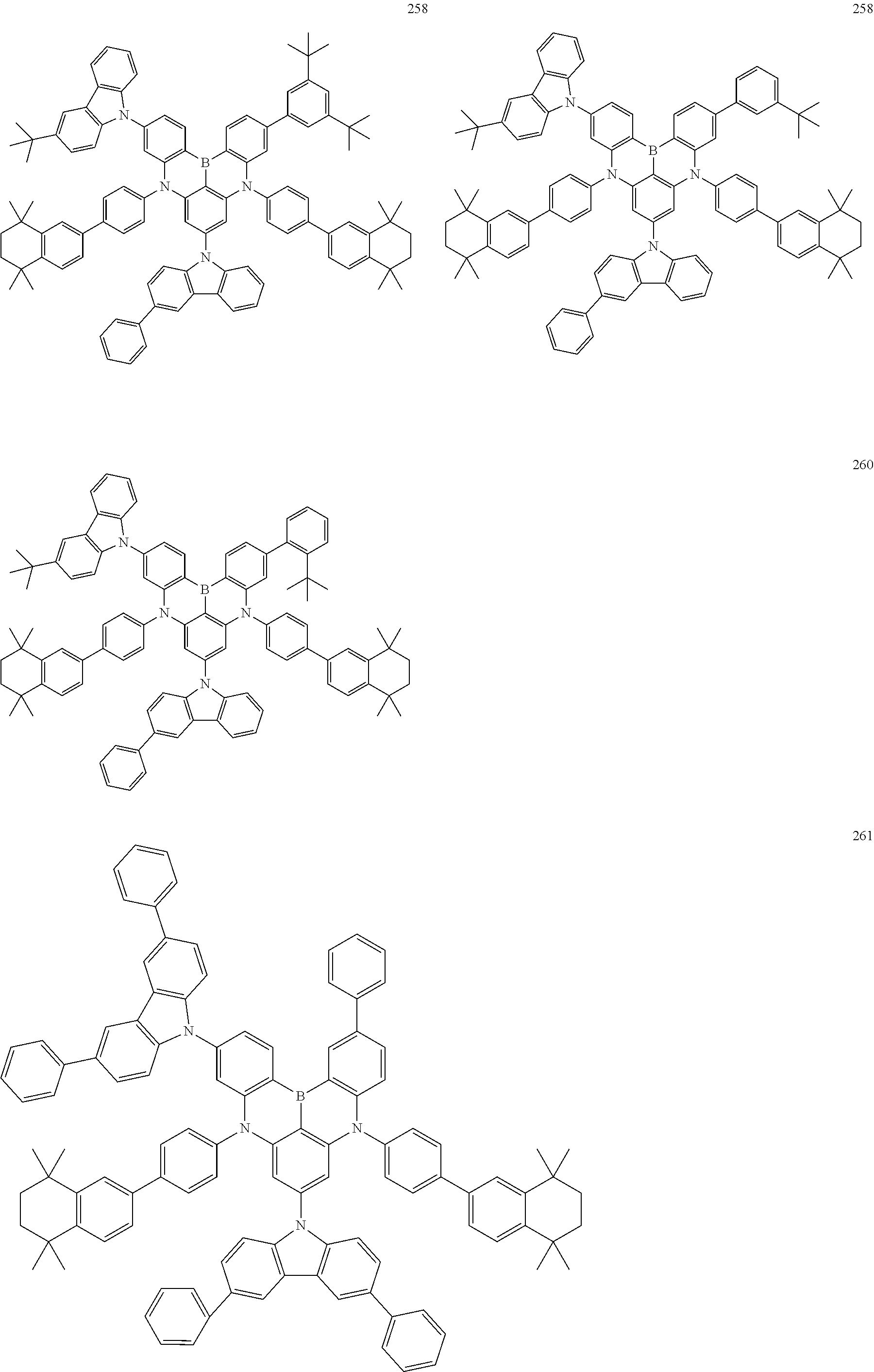

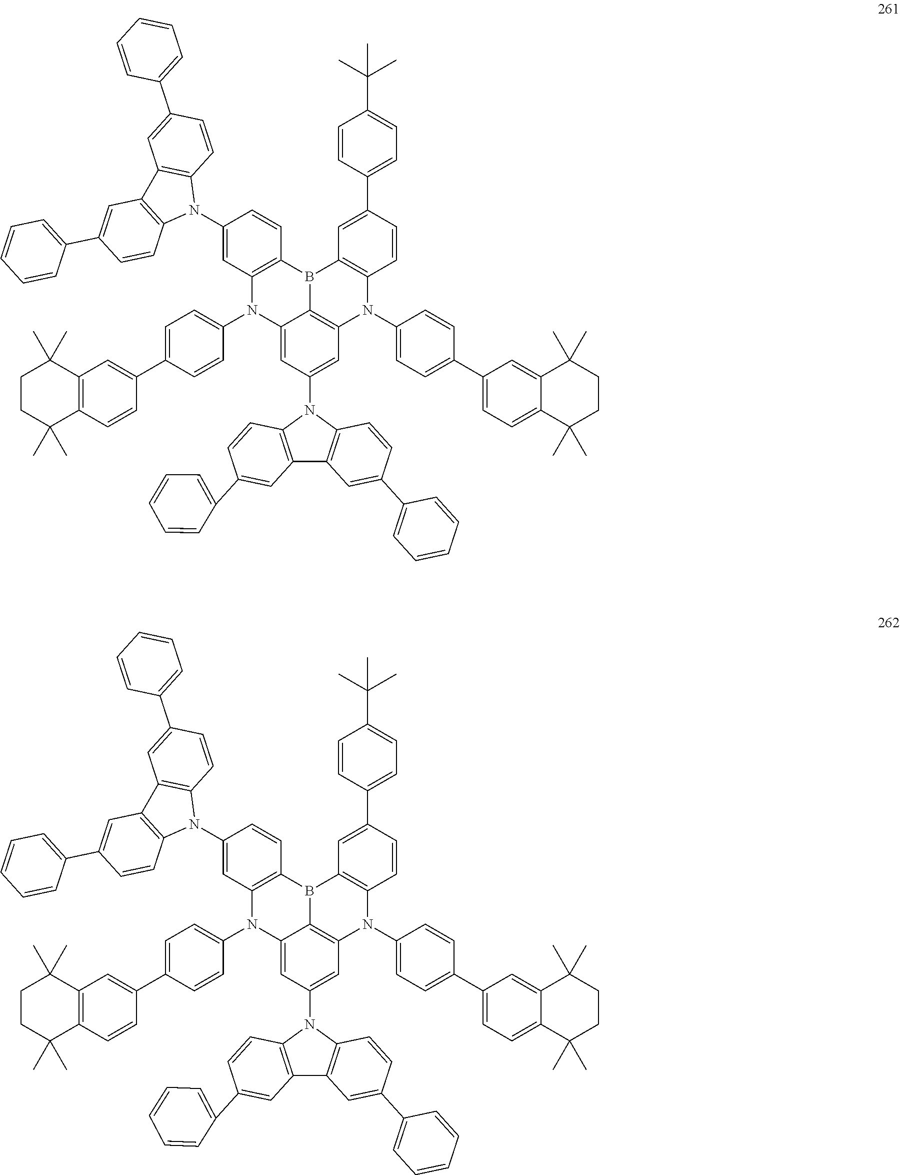

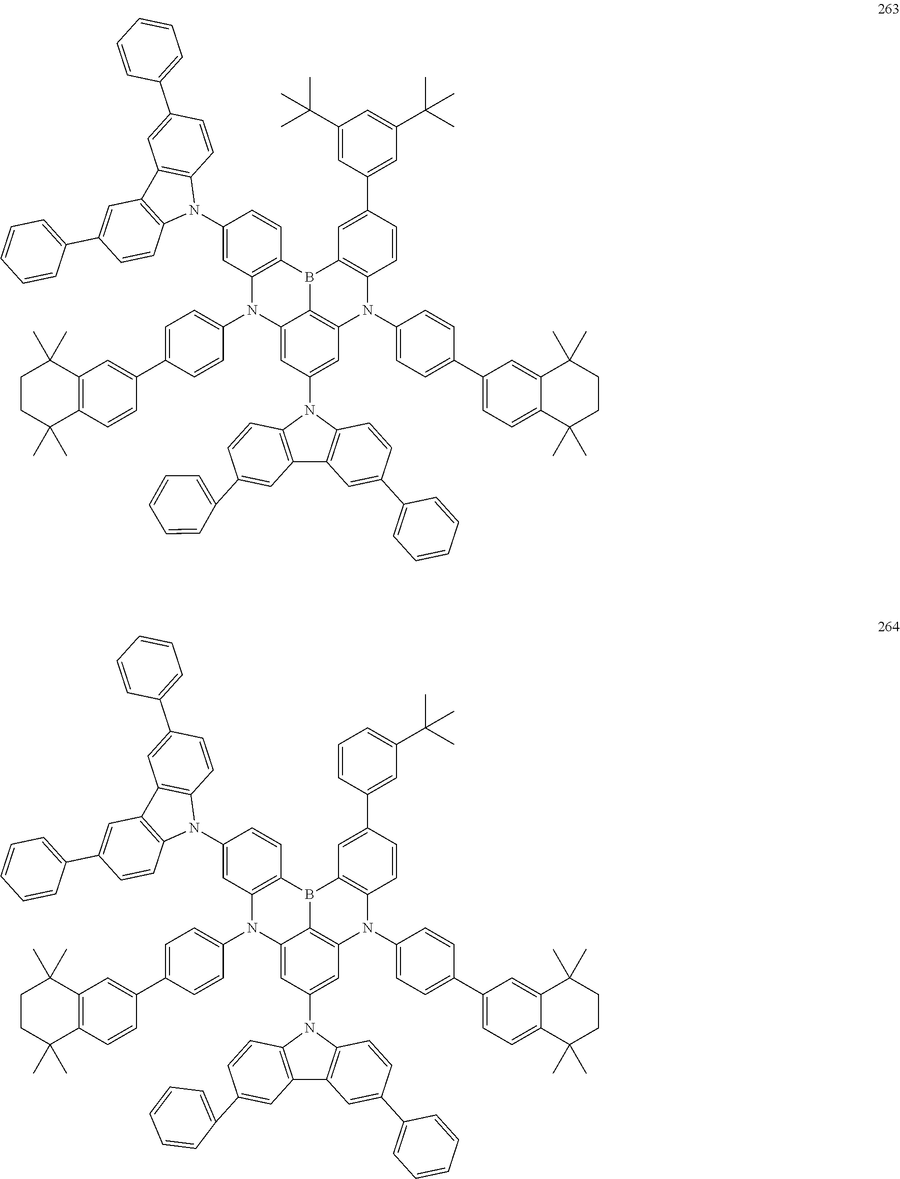 [SMILES: CC(C)(C)c1cc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CCC6(C)C)cc4)c4cc(-n5c6ccc(-c7ccccc7)cc6c6cc(-c7ccccc7)ccc65)cc(c42)N3c2ccc(-c3ccc4c(c3)C(C)(C)CCC4(C)C)cc2)cc(C(C)(C)C)c1 ; CC(C)(C)c1cccc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CCC6(C)C)cc4)c4cc(-n5c6ccc(-c7ccccc7)cc6c6cc(-c7ccccc7)ccc65)cc(c42)N3c2ccc(-c3ccc4c(c3)C(C)(C)CCC4(C)C)cc2)c1]

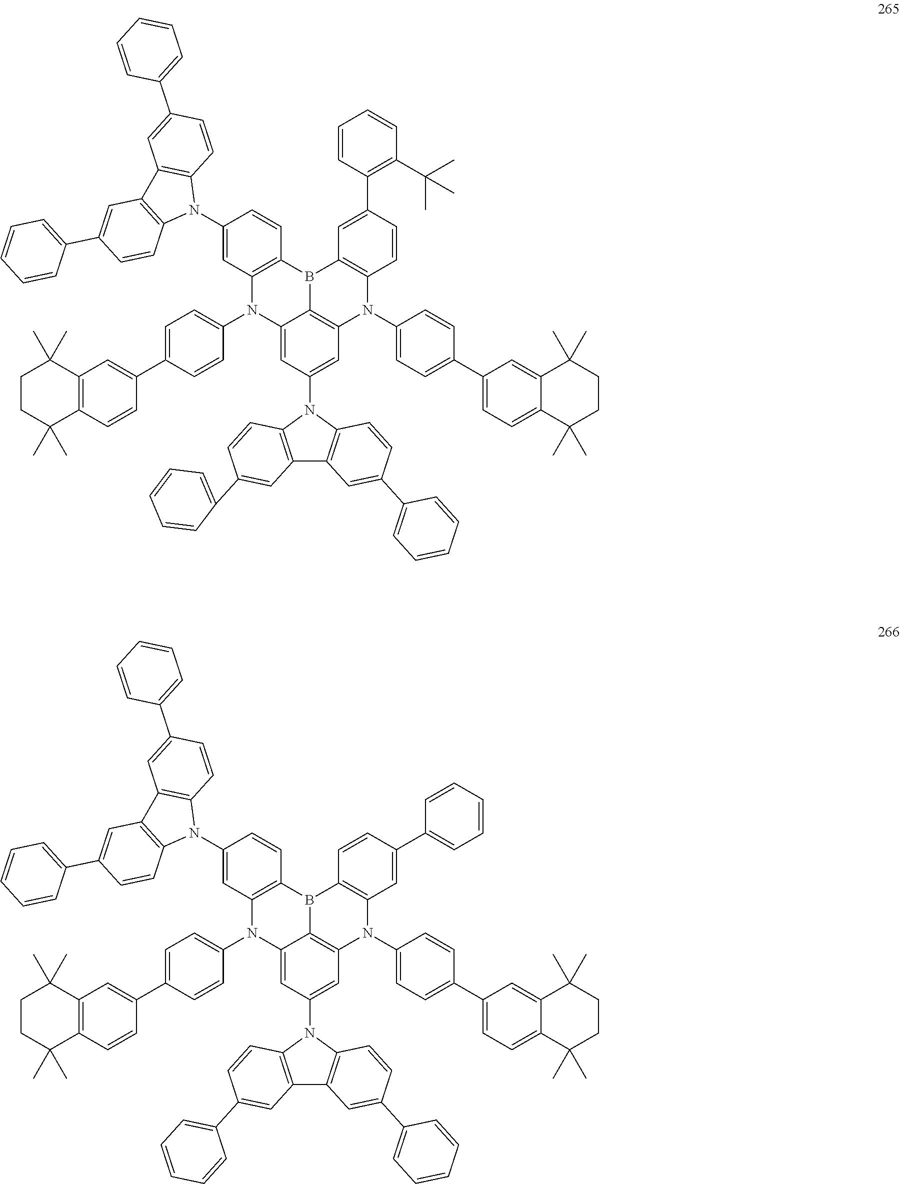 [SMILES: CC(C)(C)c1ccccc1-c1ccc2c(c1)B1c3ccc(-n4c5ccc(-c6ccccc6)cc5c5cc(-c6ccccc6)ccc54)cc3N(c3ccc(-c4ccc5c(c4)C(C)(C)CCC5(C)C)cc3)c3cc(-n4c5ccc(-c6ccccc6)cc5c5cc(-c6ccccc6)ccc54)cc(c31)N2c1ccc(-c2ccc3c(c2)C(C)(C)CCC3(C)C)cc1 ; CC1(C)CCC(C)(C)c2cc(-c3ccc(N4c5cc(-c6ccccc6)ccc5B5c6ccc(-n7c8ccc(-c9ccccc9)cc8c8cc(-c9ccccc9)ccc87)cc6N(c6ccc(-c7ccc8c(c7)C(C)(C)CCC8(C)C)cc6)c6cc(-n7c8ccc(-c9ccccc9)cc8c8cc(-c9ccccc9)ccc87)cc4c65)cc3)ccc21]

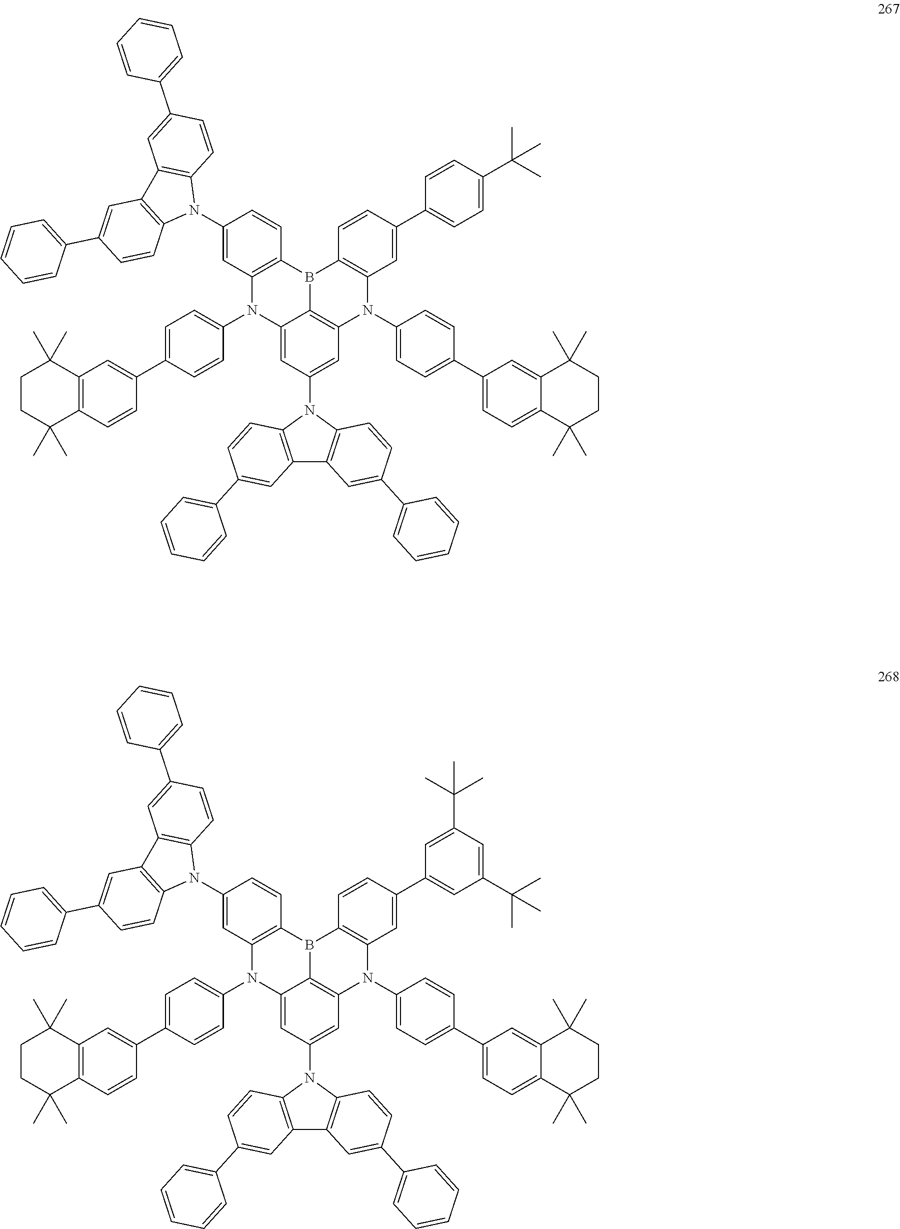 [SMILES: CC(C)(C)c1ccc(-c2ccc3c(c2)N(c2ccc(-c4ccc5c(c4)C(C)(C)CCC5(C)C)cc2)c2cc(-n4c5ccc(-c6ccccc6)cc5c5cc(-c6ccccc6)ccc54)cc4c2B3c2ccc(-n3c5ccc(-c6ccccc6)cc5c5cc(-c6ccccc6)ccc53)cc2N4c2ccc(-c3ccc4c(c3)C(C)(C)CCC4(C)C)cc2)cc1 ; CC(C)(C)c1cc(-c2ccc3c(c2)N(c2ccc(-c4ccc5c(c4)C(C)(C)CCC5(C)C)cc2)c2cc(-n4c5ccc(-c6ccccc6)cc5c5cc(-c6ccccc6)ccc54)cc4c2B3c2ccc(-n3c5ccc(-c6ccccc6)cc5c5cc(-c6ccccc6)ccc53)cc2N4c2ccc(-c3ccc4c(c3)C(C)(C)CCC4(C)C)cc2)cc(C(C)(C)C)c1]

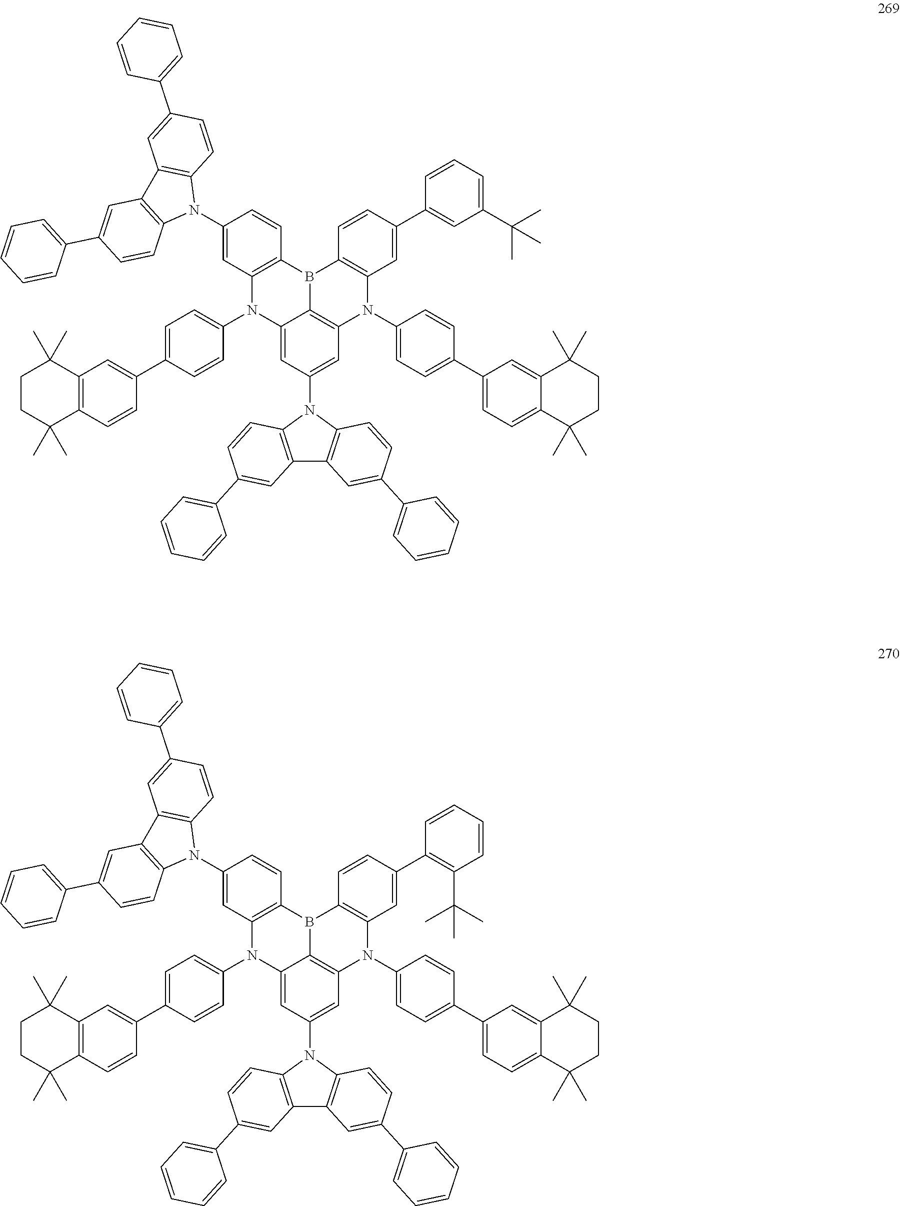 [SMILES: CC(C)(C)c1cccc(-c2ccc3c(c2)N(c2ccc(-c4ccc5c(c4)C(C)(C)CCC5(C)C)cc2)c2cc(-n4c5ccc(-c6ccccc6)cc5c5cc(-c6ccccc6)ccc54)cc4c2B3c2ccc(-n3c5ccc(-c6ccccc6)cc5c5cc(-c6ccccc6)ccc53)cc2N4c2ccc(-c3ccc4c(c3)C(C)(C)CCC4(C)C)cc2)c1 ; CC(C)(C)c1ccccc1-c1ccc2c(c1)N(c1ccc(-c3ccc4c(c3)C(C)(C)CCC4(C)C)cc1)c1cc(-n3c4ccc(-c5ccccc5)cc4c4cc(-c5ccccc5)ccc43)cc3c1B2c1ccc(-n2c4ccc(-c5ccccc5)cc4c4cc(-c5ccccc5)ccc42)cc1N3c1ccc(-c2ccc3c(c2)C(C)(C)CCC3(C)C)cc1]

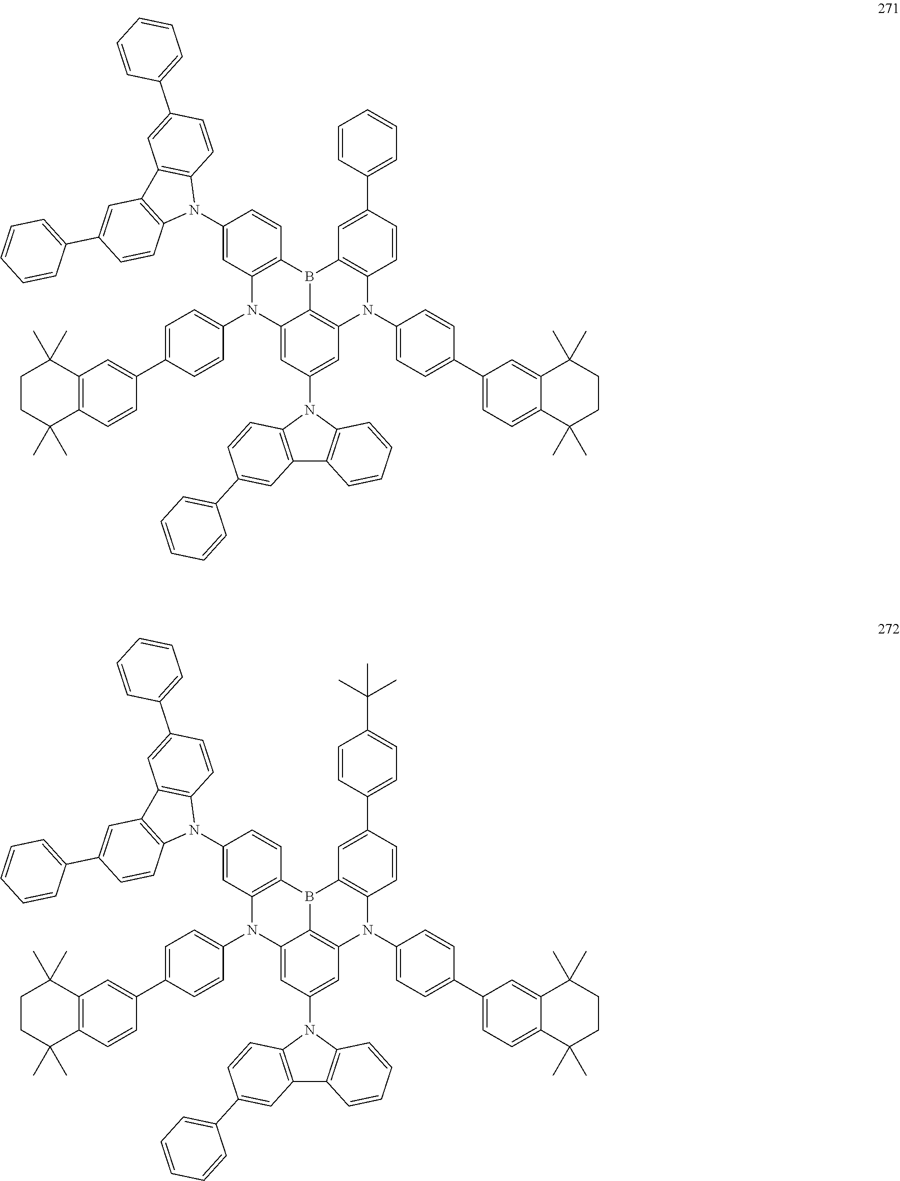 [SMILES: CC1(C)CCC(C)(C)c2cc(-c3ccc(N4c5ccc(-c6ccccc6)cc5B5c6ccc(-n7c8ccc(-c9ccccc9)cc8c8cc(-c9ccccc9)ccc87)cc6N(c6ccc(-c7ccc8c(c7)C(C)(C)CCC8(C)C)cc6)c6cc(-n7c8ccccc8c8cc(-c9ccccc9)ccc87)cc4c65)cc3)ccc21 ; CC(C)(C)c1ccc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CCC6(C)C)cc4)c4cc(-n5c6ccccc6c6cc(-c7ccccc7)ccc65)cc(c42)N3c2ccc(-c3ccc4c(c3)C(C)(C)CCC4(C)C)cc2)cc1]

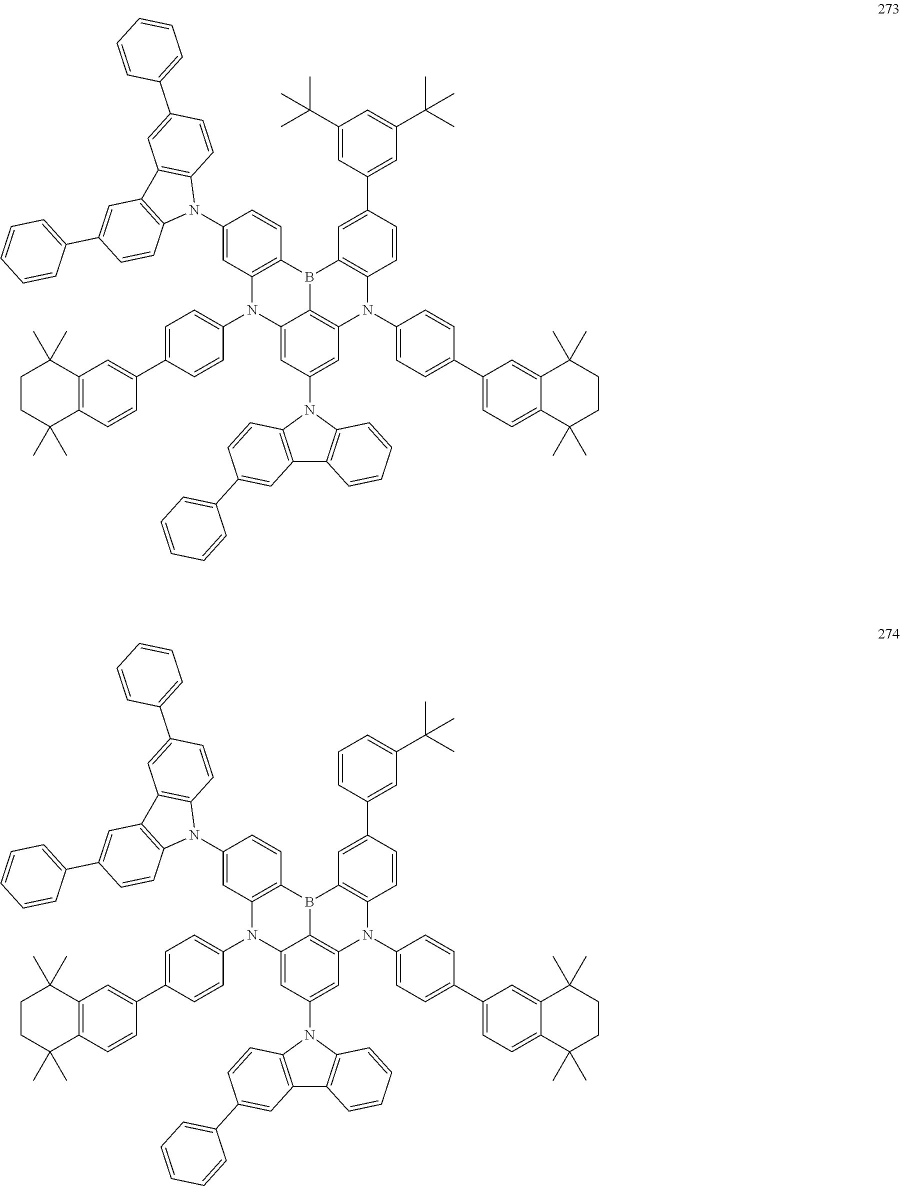 [SMILES: CC(C)(C)c1cc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CCC6(C)C)cc4)c4cc(-n5c6ccccc6c6cc(-c7ccccc7)ccc65)cc(c42)N3c2ccc(-c3ccc4c(c3)C(C)(C)CCC4(C)C)cc2)cc(C(C)(C)C)c1 ; CC(C)(C)c1cccc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CCC6(C)C)cc4)c4cc(-n5c6ccccc6c6cc(-c7ccccc7)ccc65)cc(c42)N3c2ccc(-c3ccc4c(c3)C(C)(C)CCC4(C)C)cc2)c1]

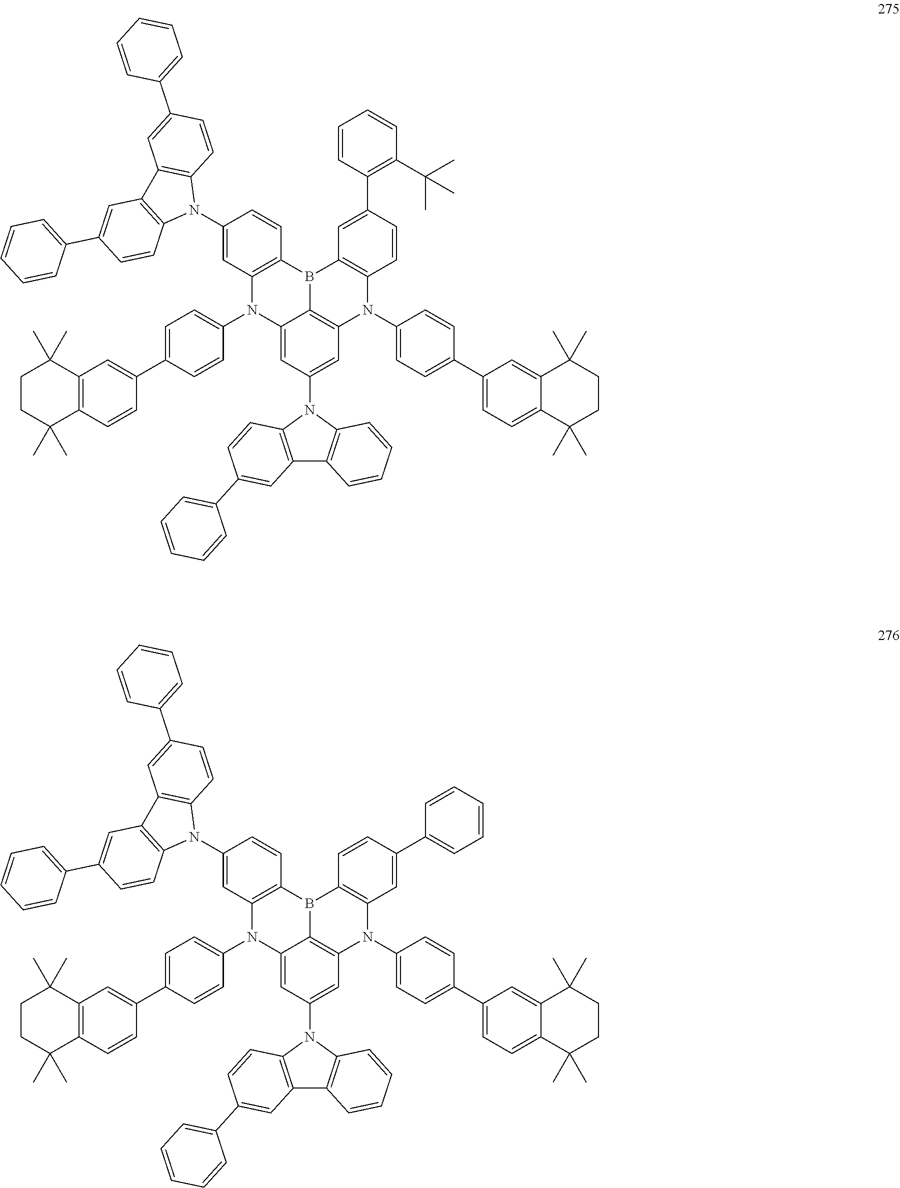 [SMILES: CC(C)(C)c1ccccc1-c1ccc2c(c1)B1c3ccc(-n4c5ccc(-c6ccccc6)cc5c5cc(-c6ccccc6)ccc54)cc3N(c3ccc(-c4ccc5c(c4)C(C)(C)CCC5(C)C)cc3)c3cc(-n4c5ccccc5c5cc(-c6ccccc6)ccc54)cc(c31)N2c1ccc(-c2ccc3c(c2)C(C)(C)CCC3(C)C)cc1 ; CC1(C)CCC(C)(C)c2cc(-c3ccc(N4c5cc(-c6ccccc6)ccc5B5c6ccc(-n7c8ccc(-c9ccccc9)cc8c8cc(-c9ccccc9)ccc87)cc6N(c6ccc(-c7ccc8c(c7)C(C)(C)CCC8(C)C)cc6)c6cc(-n7c8ccccc8c8cc(-c9ccccc9)ccc87)cc4c65)cc3)ccc21]

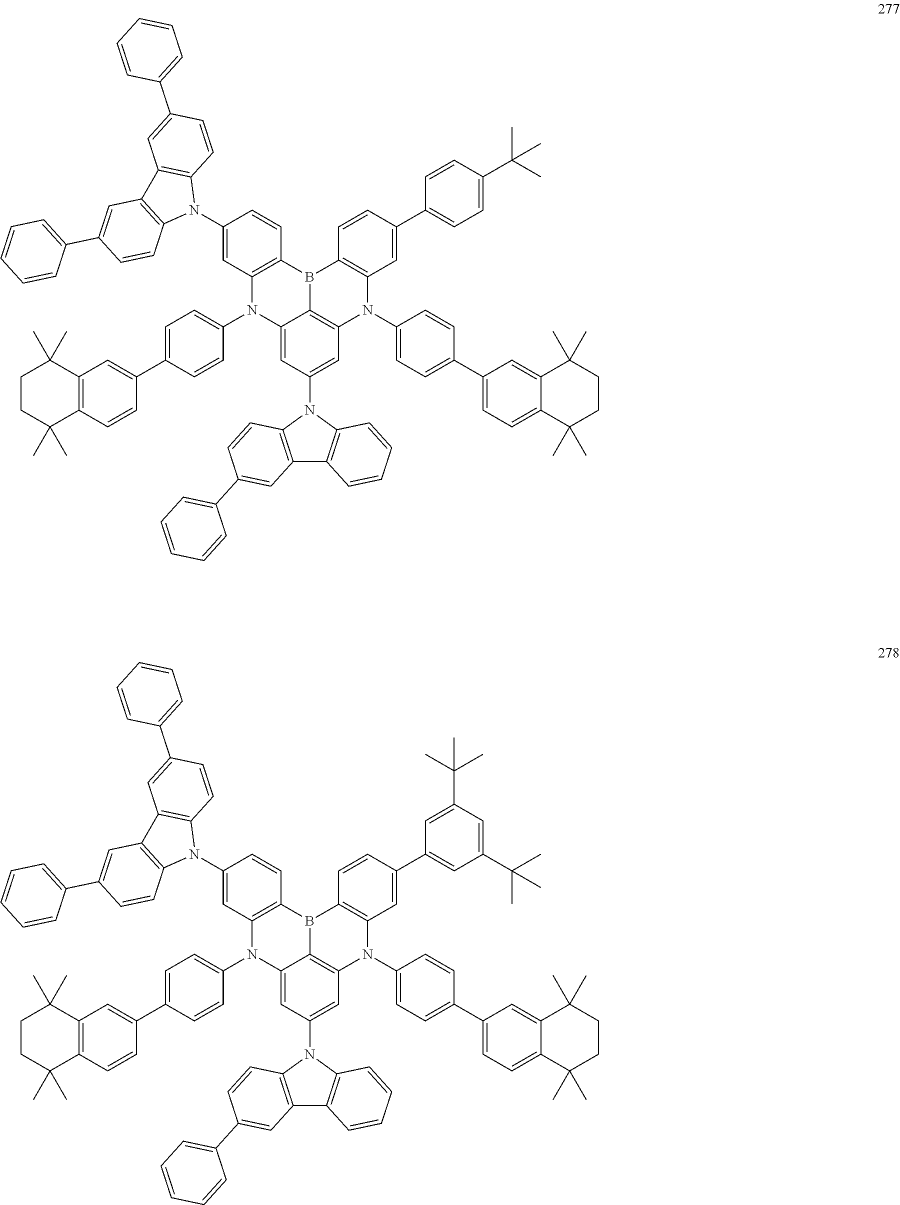 [SMILES: CC(C)(C)c1ccc(-c2ccc3c(c2)N(c2ccc(-c4ccc5c(c4)C(C)(C)CCC5(C)C)cc2)c2cc(-n4c5ccccc5c5cc(-c6ccccc6)ccc54)cc4c2B3c2ccc(-n3c5ccc(-c6ccccc6)cc5c5cc(-c6ccccc6)ccc53)cc2N4c2ccc(-c3ccc4c(c3)C(C)(C)CCC4(C)C)cc2)cc1 ; CC(C)(C)c1cc(-c2ccc3c(c2)N(c2ccc(-c4ccc5c(c4)C(C)(C)CCC5(C)C)cc2)c2cc(-n4c5ccccc5c5cc(-c6ccccc6)ccc54)cc4c2B3c2ccc(-n3c5ccc(-c6ccccc6)cc5c5cc(-c6ccccc6)ccc53)cc2N4c2ccc(-c3ccc4c(c3)C(C)(C)CCC4(C)C)cc2)cc(C(C)(C)C)c1]

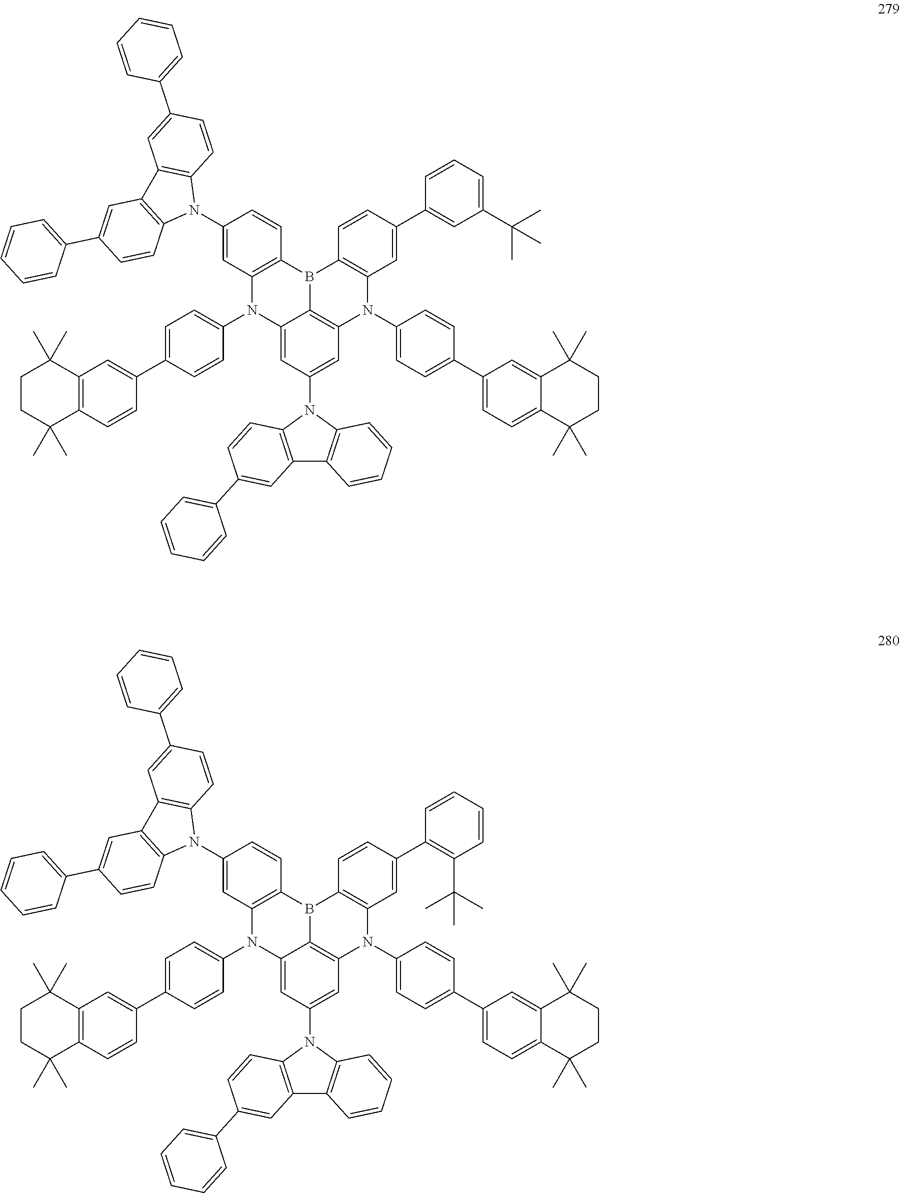 [SMILES: CC(C)(C)c1cccc(-c2ccc3c(c2)N(c2ccc(-c4ccc5c(c4)C(C)(C)CCC5(C)C)cc2)c2cc(-n4c5ccccc5c5cc(-c6ccccc6)ccc54)cc4c2B3c2ccc(-n3c5ccc(-c6ccccc6)cc5c5cc(-c6ccccc6)ccc53)cc2N4c2ccc(-c3ccc4c(c3)C(C)(C)CCC4(C)C)cc2)c1 ; CC(C)(C)c1ccccc1-c1ccc2c(c1)N(c1ccc(-c3ccc4c(c3)C(C)(C)CCC4(C)C)cc1)c1cc(-n3c4ccccc4c4cc(-c5ccccc5)ccc43)cc3c1B2c1ccc(-n2c4ccc(-c5ccccc5)cc4c4cc(-c5ccccc5)ccc42)cc1N3c1ccc(-c2ccc3c(c2)C(C)(C)CCC3(C)C)cc1]

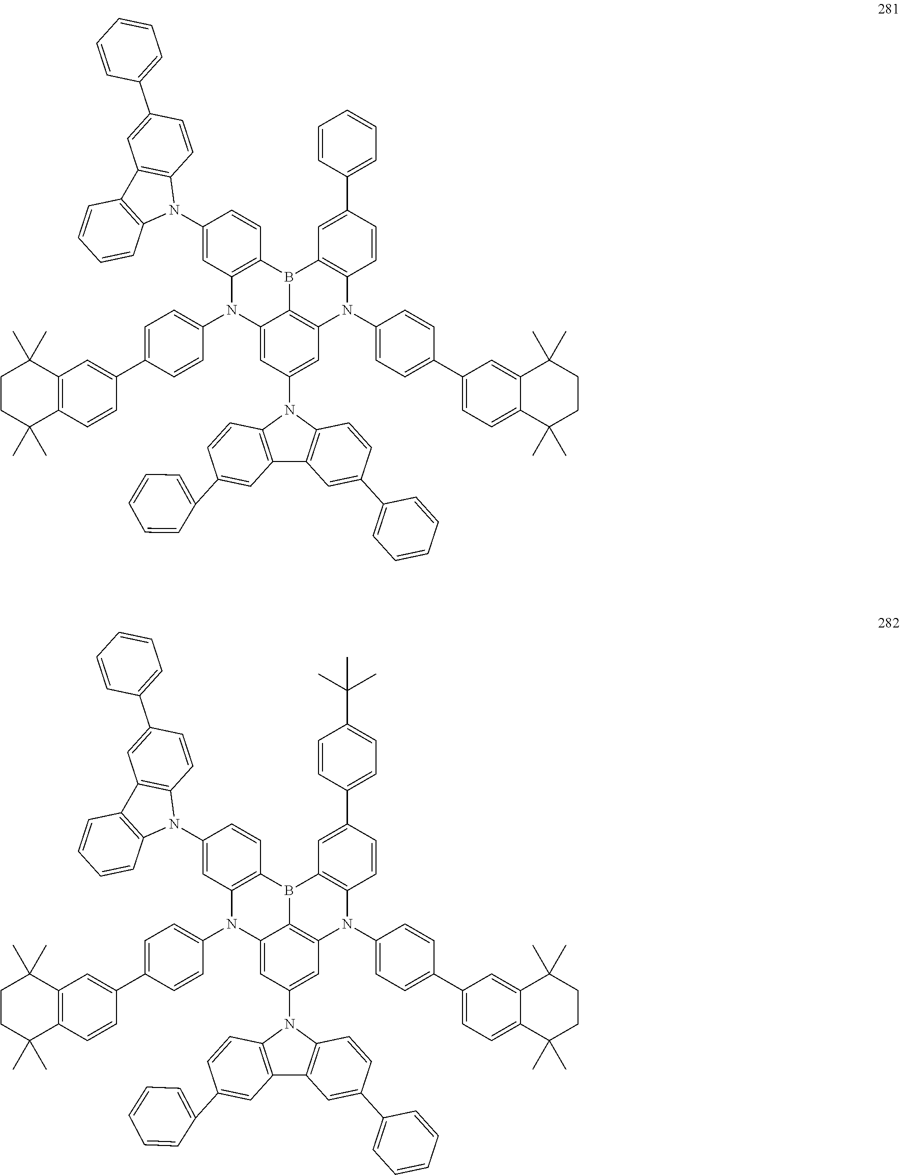

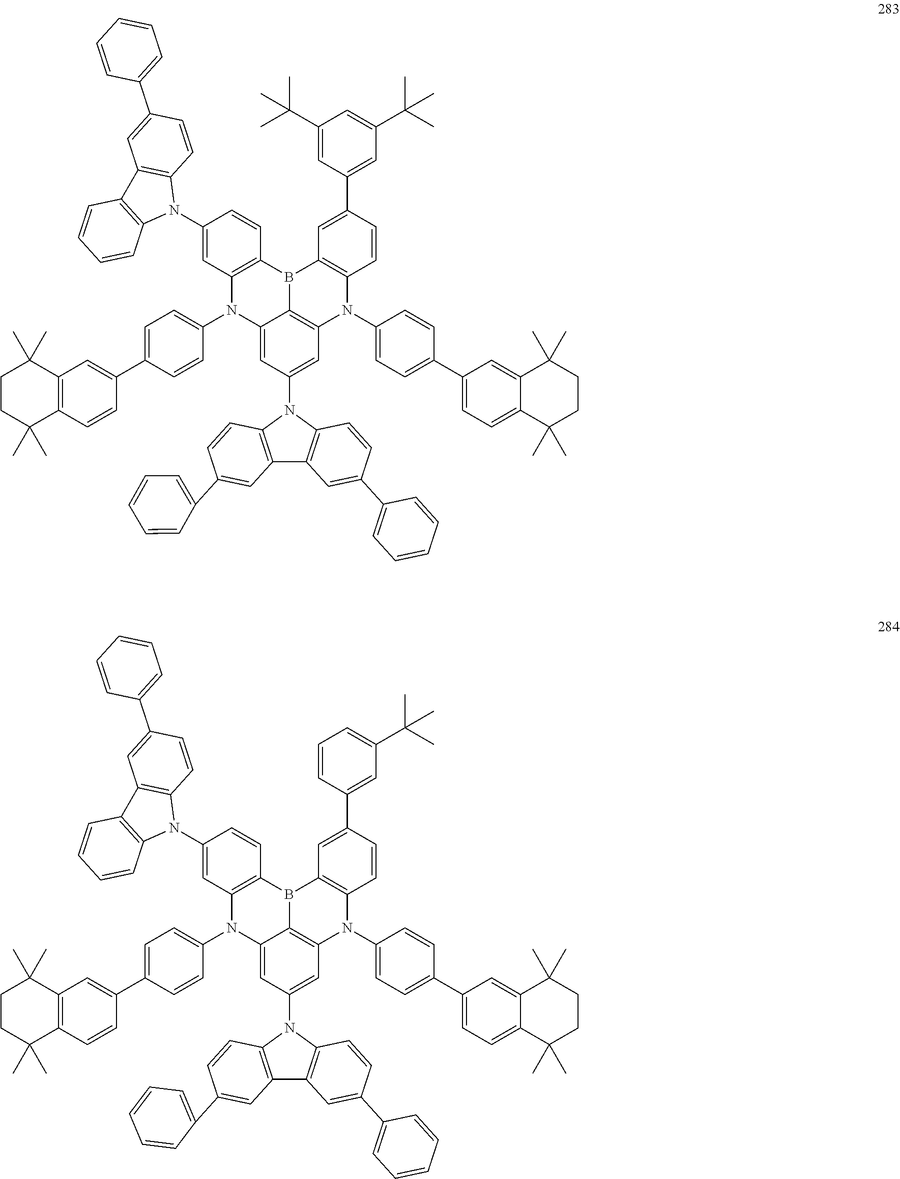 [SMILES: CC(C)(C)c1cc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccc(-c7ccccc7)cc6c6cc(-c7ccccc7)ccc65)cc(c42)N3c2ccc(-c3ccc4c(c3)C(C)(C)CCC4(C)C)cc2)cc(C(C)(C)C)c1 ; CC(C)(C)c1cccc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccc(-c7ccccc7)cc6c6cc(-c7ccccc7)ccc65)cc(c42)N3c2ccc(-c3ccc4c(c3)C(C)(C)CCC4(C)C)cc2)c1]

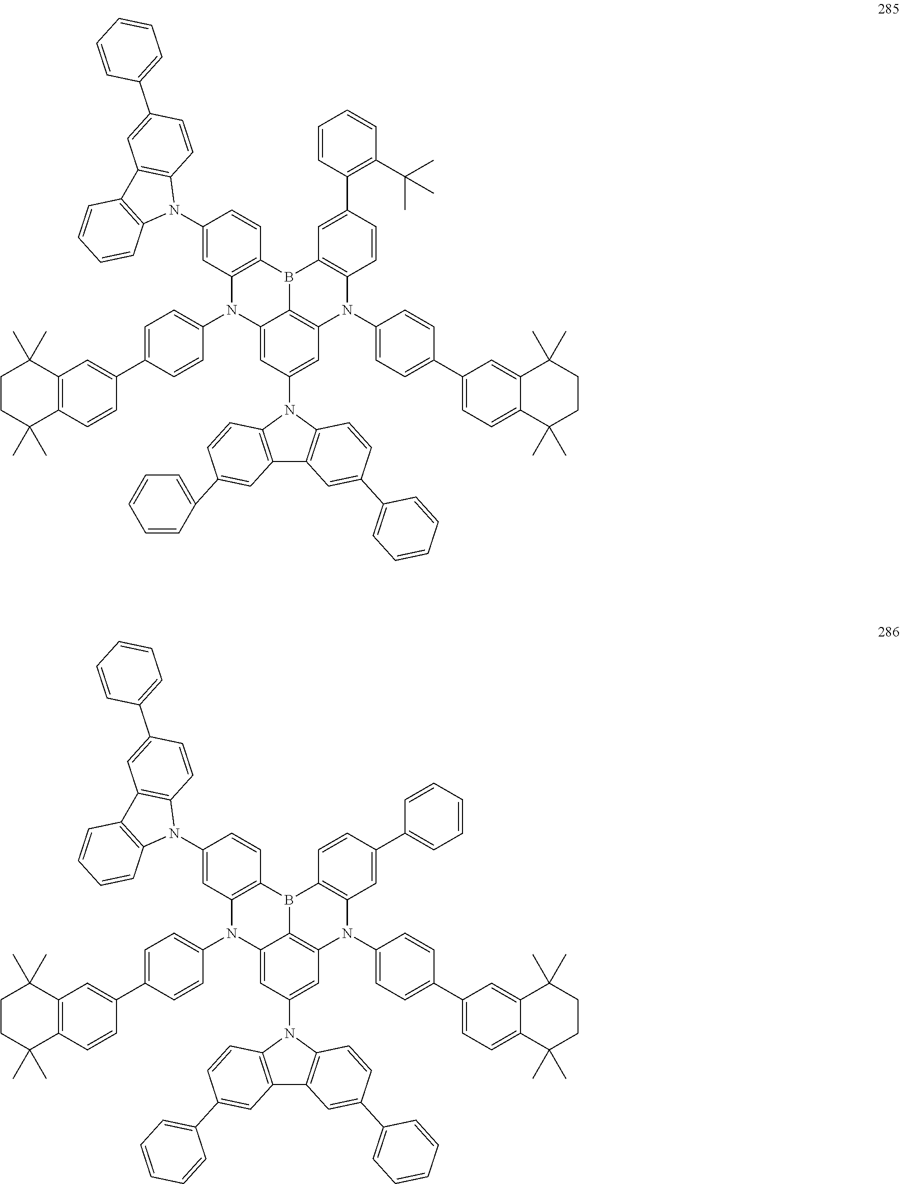 [SMILES: CC(C)(C)c1ccccc1-c1ccc2c(c1)B1c3ccc(-n4c5ccccc5c5cc(-c6ccccc6)ccc54)cc3N(c3ccc(-c4ccc5c(c4)C(C)(C)CCC5(C)C)cc3)c3cc(-n4c5ccc(-c6ccccc6)cc5c5cc(-c6ccccc6)ccc54)cc(c31)N2c1ccc(-c2ccc3c(c2)C(C)(C)CCC3(C)C)cc1 ; CC1(C)CCC(C)(C)c2cc(-c3ccc(N4c5cc(-c6ccccc6)ccc5B5c6ccc(-n7c8ccccc8c8cc(-c9ccccc9)ccc87)cc6N(c6ccc(-c7ccc8c(c7)C(C)(C)CCC8(C)C)cc6)c6cc(-n7c8ccc(-c9ccccc9)cc8c8cc(-c9ccccc9)ccc87)cc4c65)cc3)ccc21]

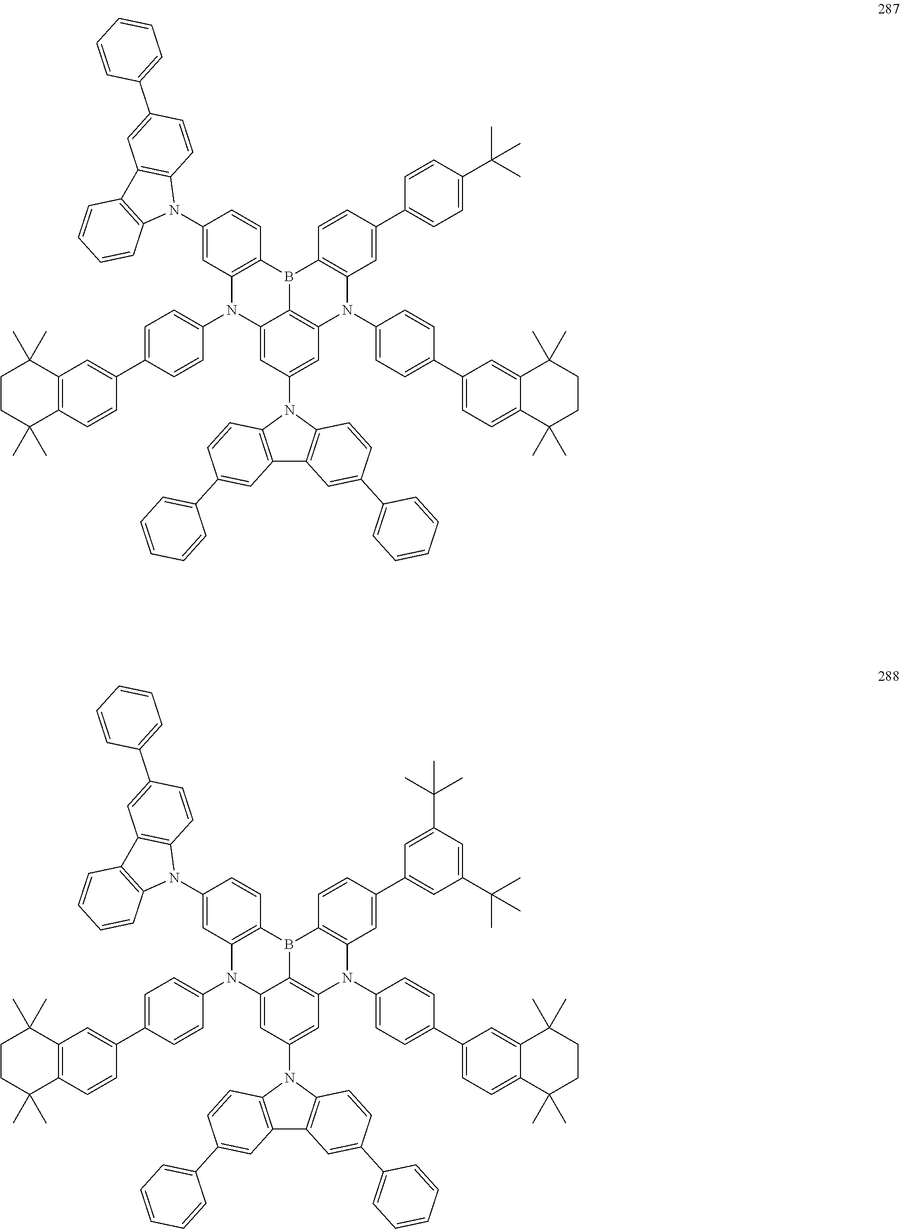 [SMILES: CC(C)(C)c1ccc(-c2ccc3c(c2)N(c2ccc(-c4ccc5c(c4)C(C)(C)CCC5(C)C)cc2)c2cc(-n4c5ccc(-c6ccccc6)cc5c5cc(-c6ccccc6)ccc54)cc4c2B3c2ccc(-n3c5ccccc5c5cc(-c6ccccc6)ccc53)cc2N4c2ccc(-c3ccc4c(c3)C(C)(C)CCC4(C)C)cc2)cc1 ; CC(C)(C)c1cc(-c2ccc3c(c2)N(c2ccc(-c4ccc5c(c4)C(C)(C)CCC5(C)C)cc2)c2cc(-n4c5ccc(-c6ccccc6)cc5c5cc(-c6ccccc6)ccc54)cc4c2B3c2ccc(-n3c5ccccc5c5cc(-c6ccccc6)ccc53)cc2N4c2ccc(-c3ccc4c(c3)C(C)(C)CCC4(C)C)cc2)cc(C(C)(C)C)c1]

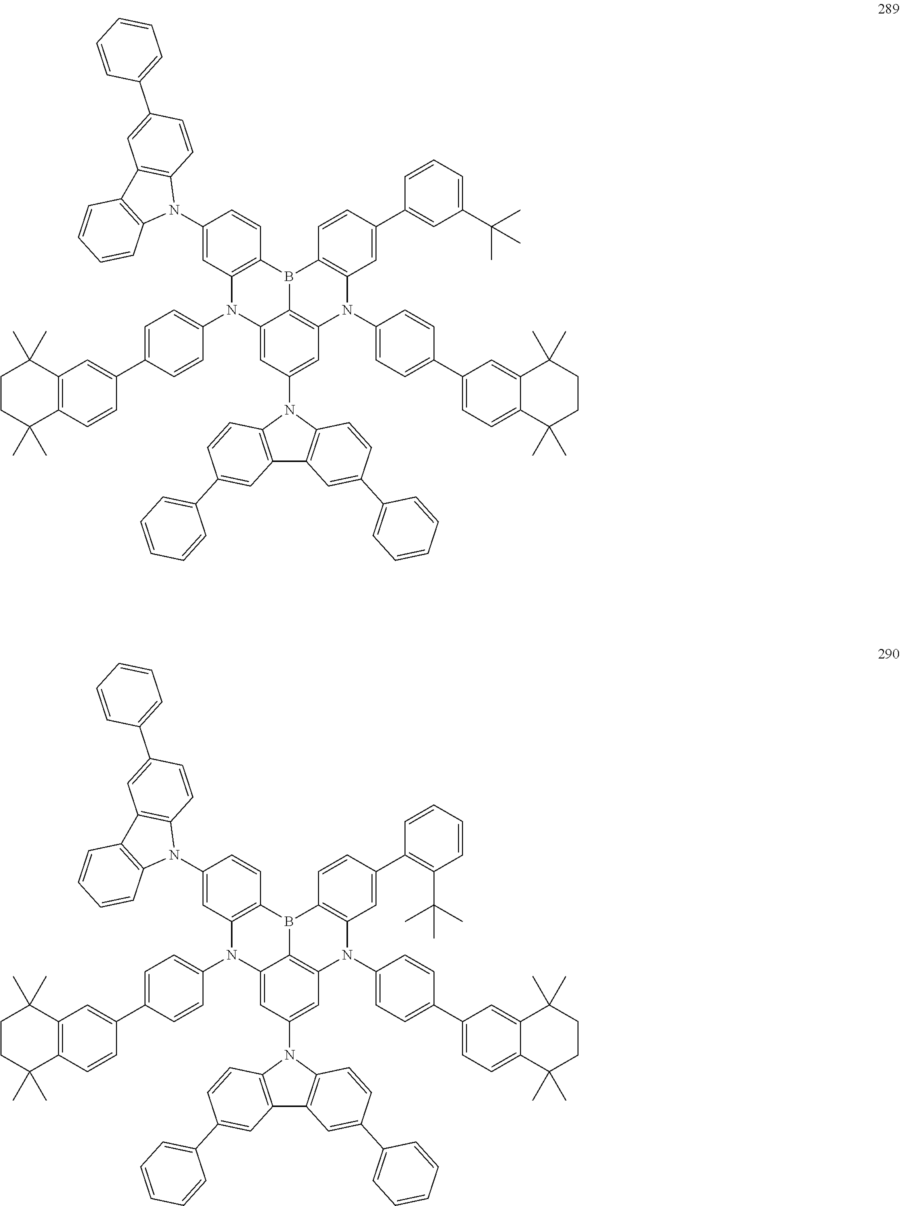 [SMILES: CC(C)(C)c1cccc(-c2ccc3c(c2)N(c2ccc(-c4ccc5c(c4)C(C)(C)CCC5(C)C)cc2)c2cc(-n4c5ccc(-c6ccccc6)cc5c5cc(-c6ccccc6)ccc54)cc4c2B3c2ccc(-n3c5ccccc5c5cc(-c6ccccc6)ccc53)cc2N4c2ccc(-c3ccc4c(c3)C(C)(C)CCC4(C)C)cc2)c1 ; CC(C)(C)c1ccccc1-c1ccc2c(c1)N(c1ccc(-c3ccc4c(c3)C(C)(C)CCC4(C)C)cc1)c1cc(-n3c4ccc(-c5ccccc5)cc4c4cc(-c5ccccc5)ccc43)cc3c1B2c1ccc(-n2c4ccccc4c4cc(-c5ccccc5)ccc42)cc1N3c1ccc(-c2ccc3c(c2)C(C)(C)CCC3(C)C)cc1]

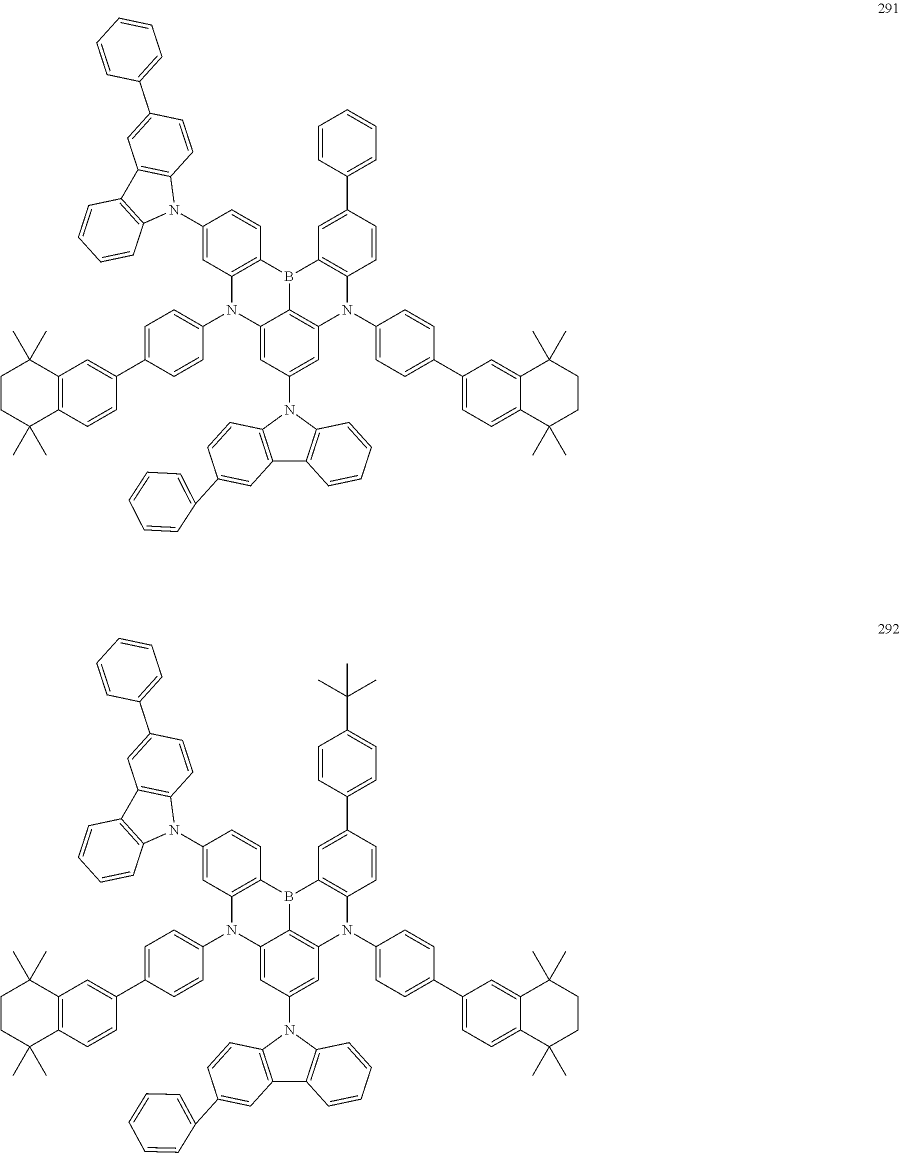 [SMILES: CC1(C)CCC(C)(C)c2cc(-c3ccc(N4c5ccc(-c6ccccc6)cc5B5c6ccc(-n7c8ccccc8c8cc(-c9ccccc9)ccc87)cc6N(c6ccc(-c7ccc8c(c7)C(C)(C)CCC8(C)C)cc6)c6cc(-n7c8ccccc8c8cc(-c9ccccc9)ccc87)cc4c65)cc3)ccc21 ; CC(C)(C)c1ccc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccccc6c6cc(-c7ccccc7)ccc65)cc(c42)N3c2ccc(-c3ccc4c(c3)C(C)(C)CCC4(C)C)cc2)cc1]

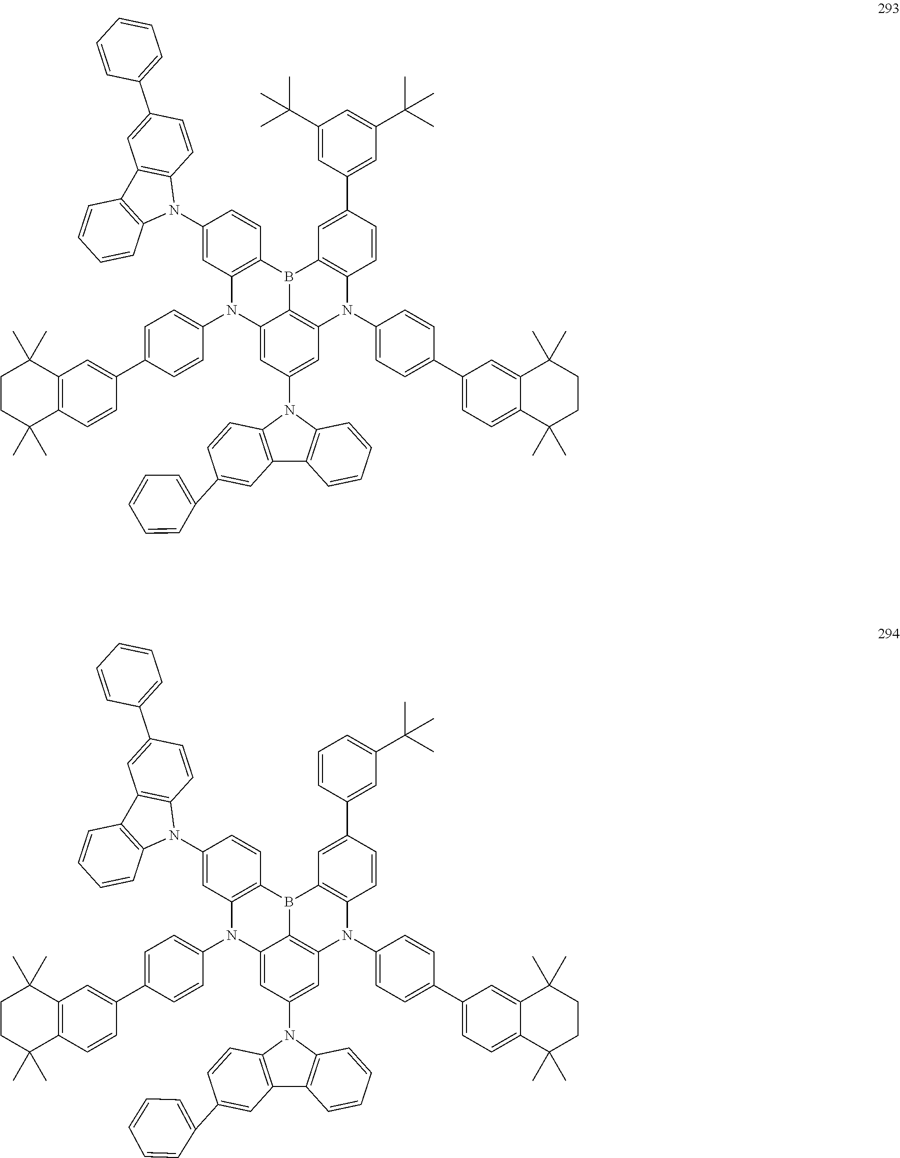 [SMILES: CC(C)(C)c1cc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccccc6c6cc(-c7ccccc7)ccc65)cc(c42)N3c2ccc(-c3ccc4c(c3)C(C)(C)CCC4(C)C)cc2)cc(C(C)(C)C)c1 ; CC(C)(C)c1cccc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccccc6c6cc(-c7ccccc7)ccc65)cc(c42)N3c2ccc(-c3ccc4c(c3)C(C)(C)CCC4(C)C)cc2)c1]

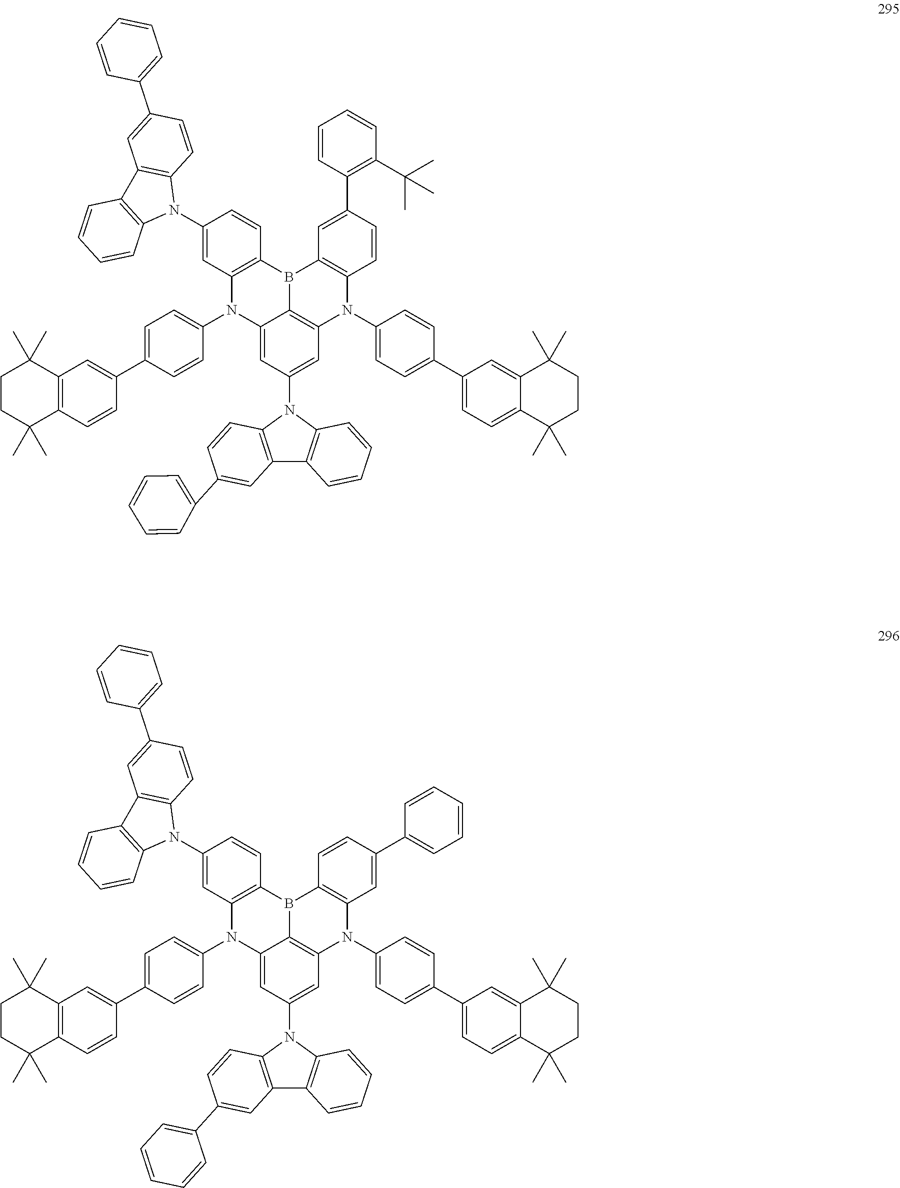 [SMILES: CC(C)(C)c1ccccc1-c1ccc2c(c1)B1c3ccc(-n4c5ccccc5c5cc(-c6ccccc6)ccc54)cc3N(c3ccc(-c4ccc5c(c4)C(C)(C)CCC5(C)C)cc3)c3cc(-n4c5ccccc5c5cc(-c6ccccc6)ccc54)cc(c31)N2c1ccc(-c2ccc3c(c2)C(C)(C)CCC3(C)C)cc1 ; CC1(C)CCC(C)(C)c2cc(-c3ccc(N4c5cc(-c6ccccc6)ccc5B5c6ccc(-n7c8ccccc8c8cc(-c9ccccc9)ccc87)cc6N(c6ccc(-c7ccc8c(c7)C(C)(C)CCC8(C)C)cc6)c6cc(-n7c8ccccc8c8cc(-c9ccccc9)ccc87)cc4c65)cc3)ccc21]

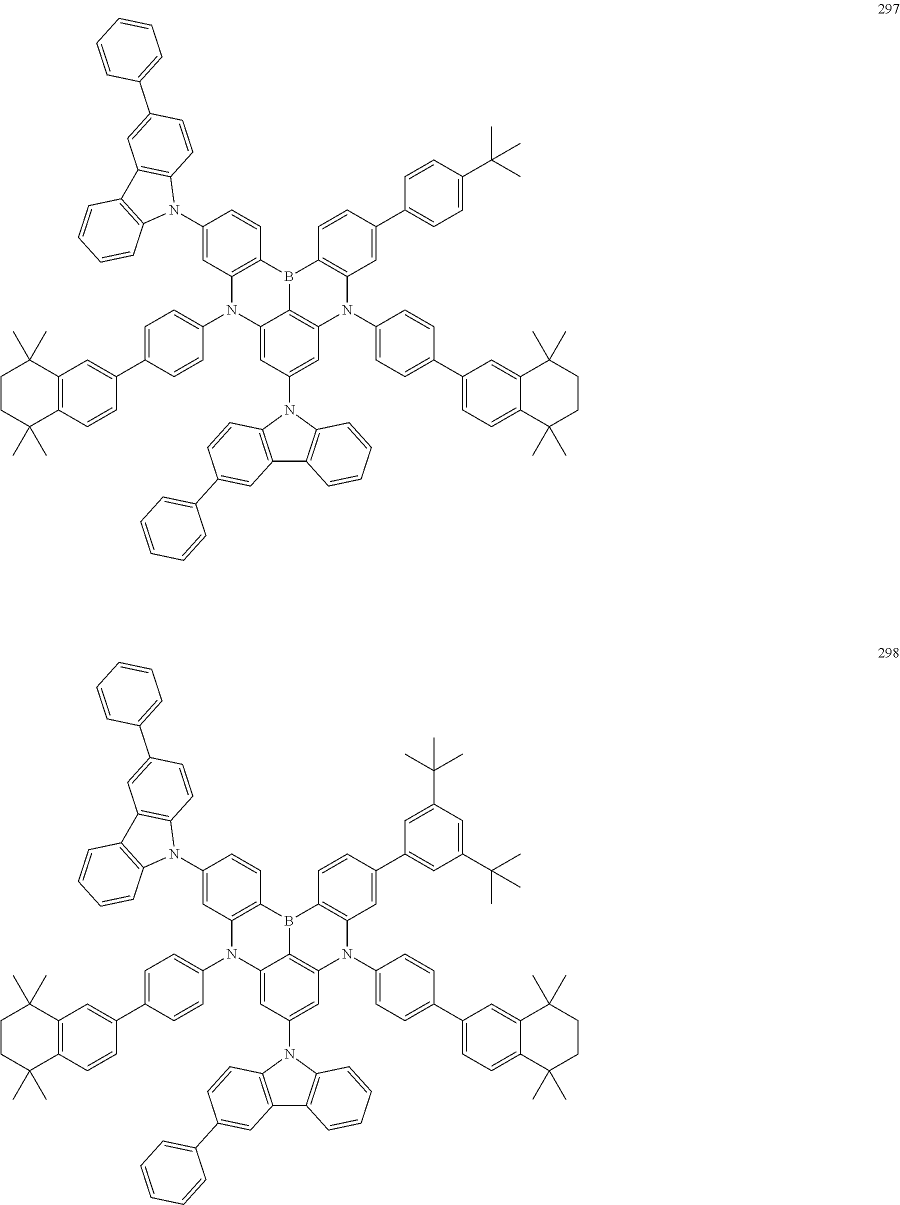 [SMILES: CC(C)(C)c1ccc(-c2ccc3c(c2)N(c2ccc(-c4ccc5c(c4)C(C)(C)CCC5(C)C)cc2)c2cc(-n4c5ccccc5c5cc(-c6ccccc6)ccc54)cc4c2B3c2ccc(-n3c5ccccc5c5cc(-c6ccccc6)ccc53)cc2N4c2ccc(-c3ccc4c(c3)C(C)(C)CCC4(C)C)cc2)cc1 ; CC(C)(C)c1cc(-c2ccc3c(c2)N(c2ccc(-c4ccc5c(c4)C(C)(C)CCC5(C)C)cc2)c2cc(-n4c5ccccc5c5cc(-c6ccccc6)ccc54)cc4c2B3c2ccc(-n3c5ccccc5c5cc(-c6ccccc6)ccc53)cc2N4c2ccc(-c3ccc4c(c3)C(C)(C)CCC4(C)C)cc2)cc(C(C)(C)C)c1]

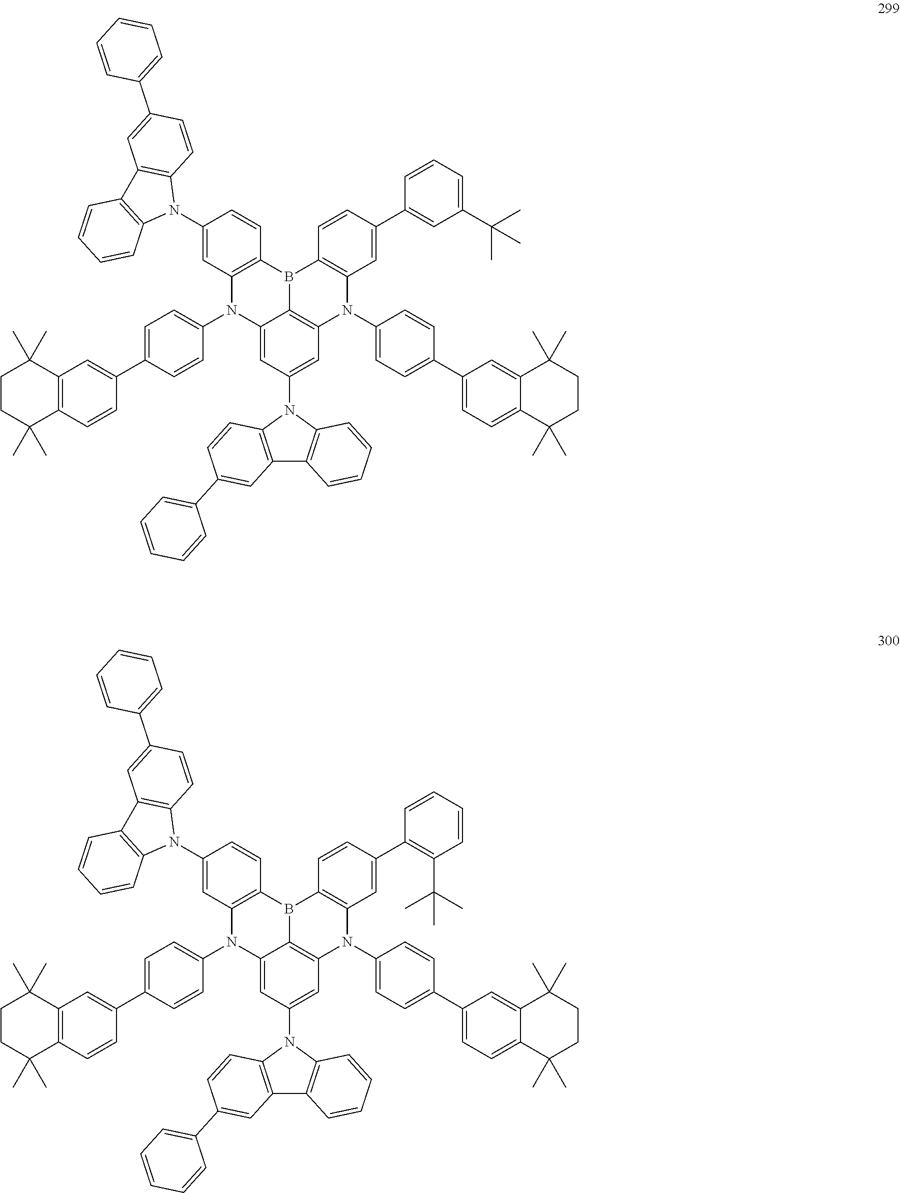 [SMILES: CC(C)(C)c1cccc(-c2ccc3c(c2)N(c2ccc(-c4ccc5c(c4)C(C)(C)CCC5(C)C)cc2)c2cc(-n4c5ccccc5c5cc(-c6ccccc6)ccc54)cc4c2B3c2ccc(-n3c5ccccc5c5cc(-c6ccccc6)ccc53)cc2N4c2ccc(-c3ccc4c(c3)C(C)(C)CCC4(C)C)cc2)c1 ; CC(C)(C)c1ccccc1-c1ccc2c(c1)N(c1ccc(-c3ccc4c(c3)C(C)(C)CCC4(C)C)cc1)c1cc(-n3c4ccccc4c4cc(-c5ccccc5)ccc43)cc3c1B2c1ccc(-n2c4ccccc4c4cc(-c5ccccc5)ccc42)cc1N3c1ccc(-c2ccc3c(c2)C(C)(C)CCC3(C)C)cc1]

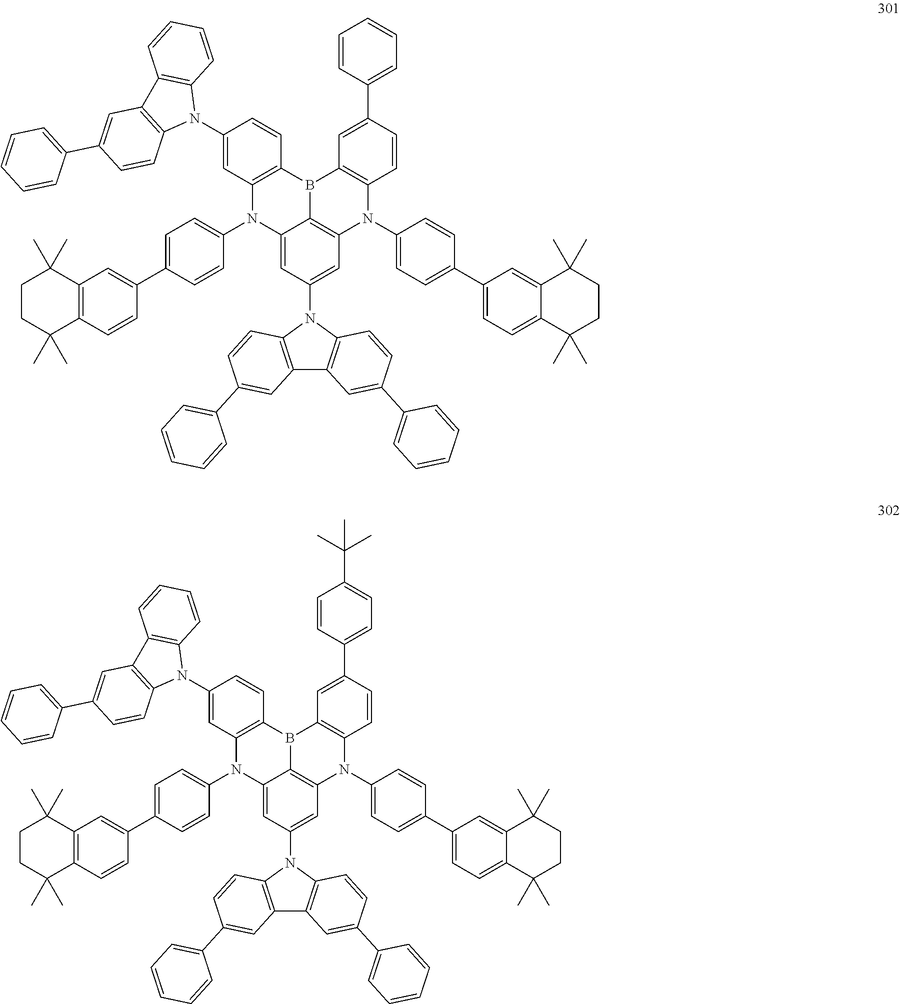 [SMILES: CC1(C)CCC(C)(C)c2cc(-c3ccc(N4c5ccc(-c6ccccc6)cc5B5c6ccc(-n7c8ccccc8c8cc(-c9ccccc9)ccc87)cc6N(c6ccc(-c7ccc8c(c7)C(C)(C)CCC8(C)C)cc6)c6cc(-n7c8ccc(-c9ccccc9)cc8c8cc(-c9ccccc9)ccc87)cc4c65)cc3)ccc21 ; CC(C)(C)c1ccc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccc(-c7ccccc7)cc6c6cc(-c7ccccc7)ccc65)cc(c42)N3c2ccc(-c3ccc4c(c3)C(C)(C)CCC4(C)C)cc2)cc1]

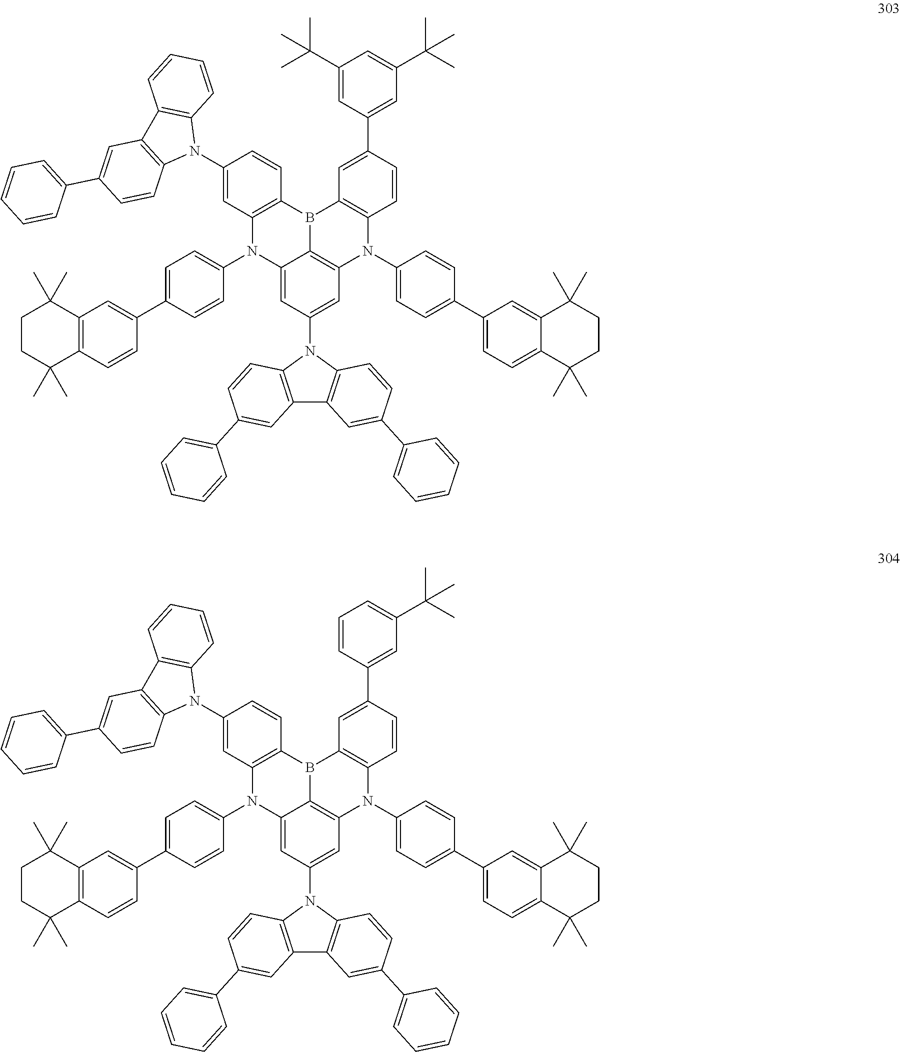 [SMILES: CC(C)(C)c1cc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccc(-c7ccccc7)cc6c6cc(-c7ccccc7)ccc65)cc(c42)N3c2ccc(-c3ccc4c(c3)C(C)(C)CCC4(C)C)cc2)cc(C(C)(C)C)c1 ; CC(C)(C)c1cccc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccc(-c7ccccc7)cc6c6cc(-c7ccccc7)ccc65)cc(c42)N3c2ccc(-c3ccc4c(c3)C(C)(C)CCC4(C)C)cc2)c1]

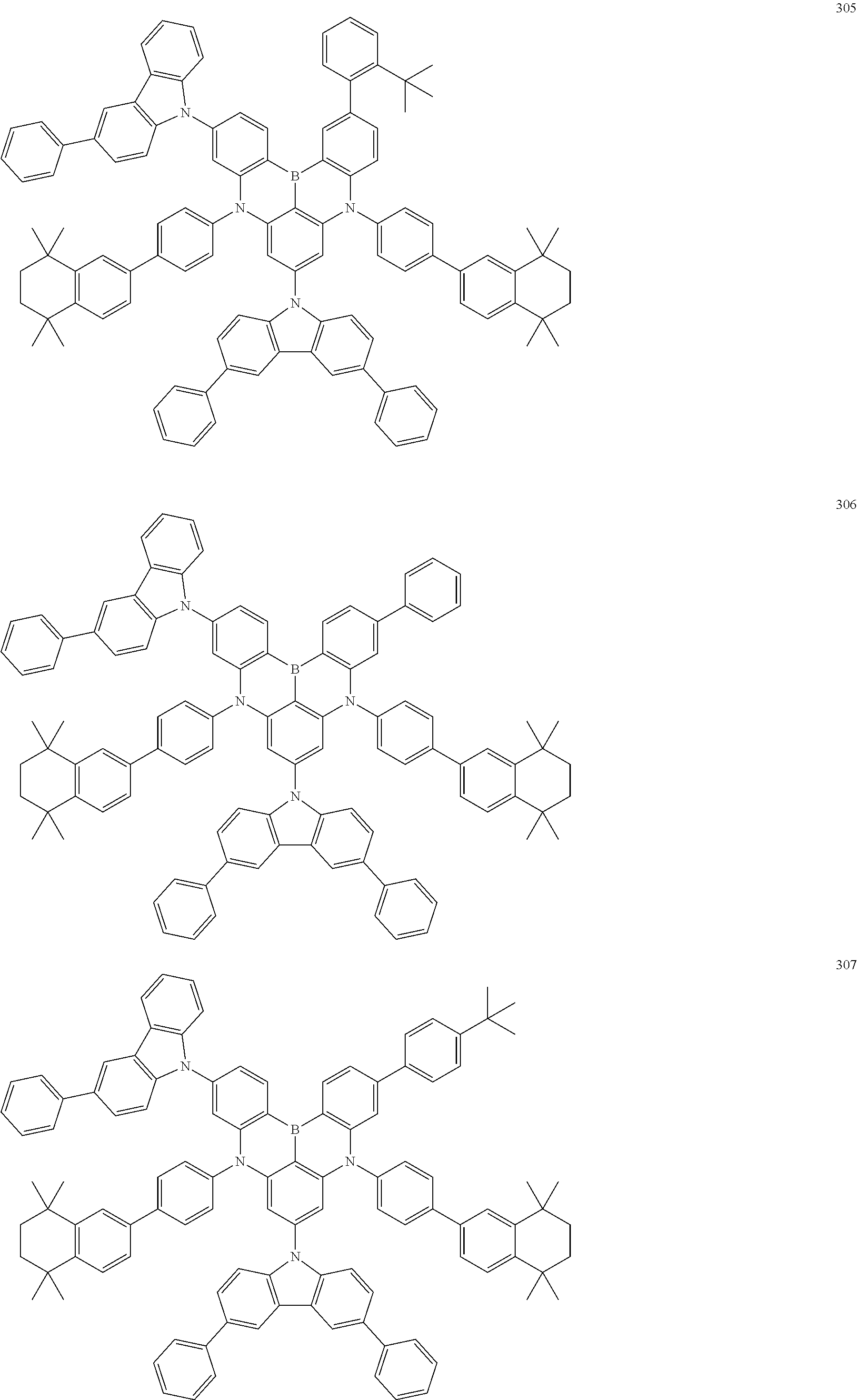

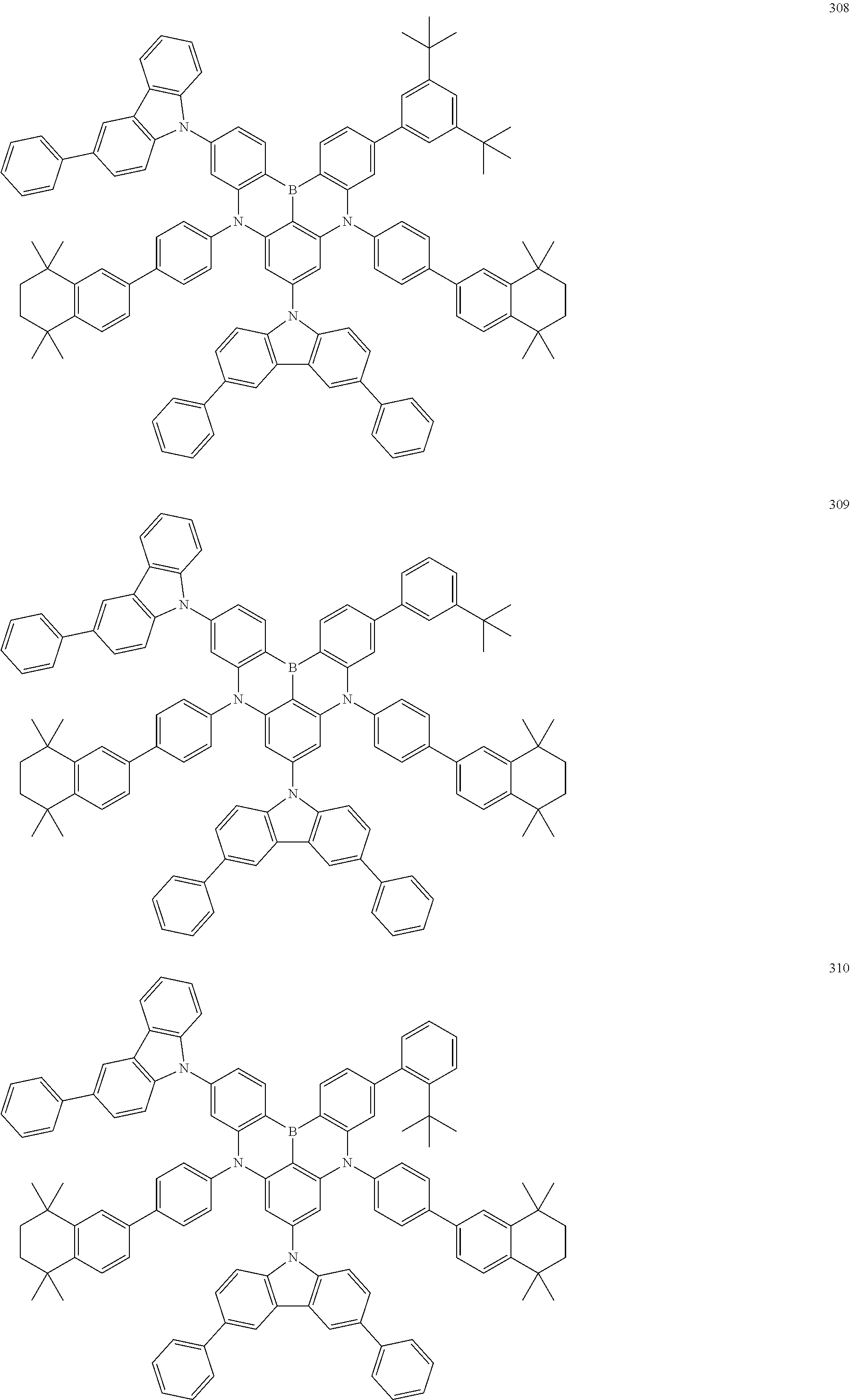

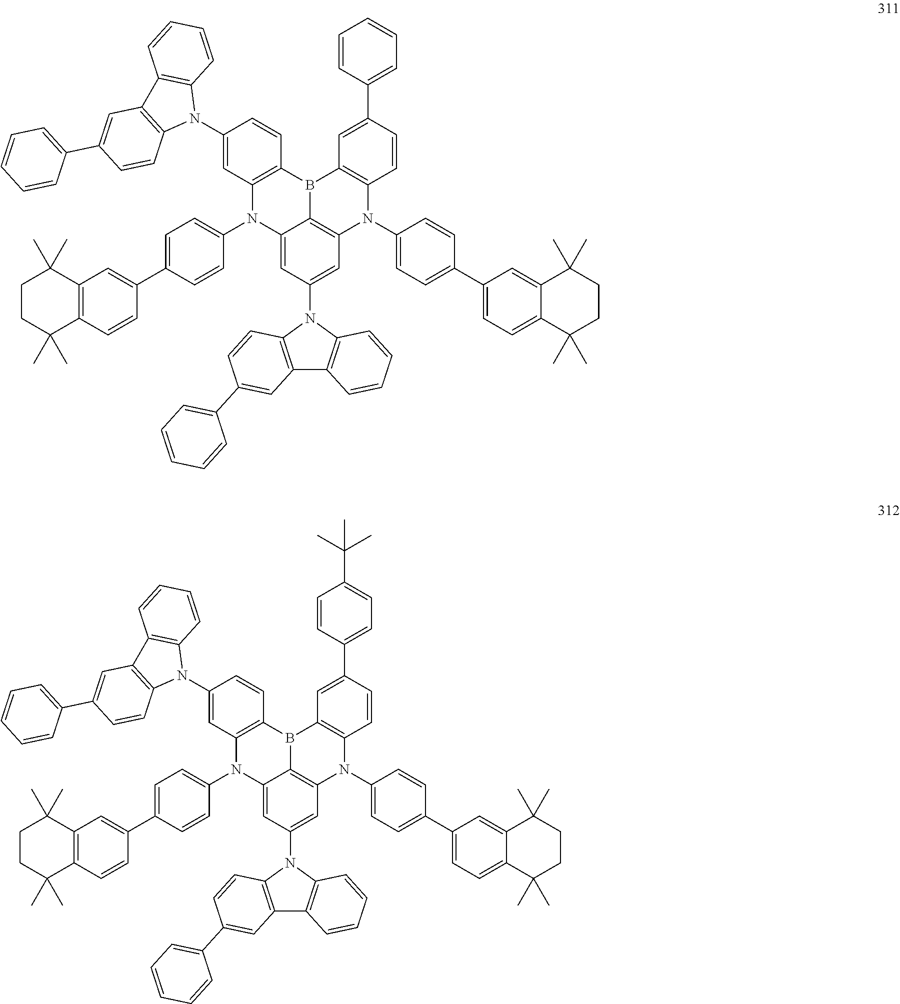 [SMILES: CC1(C)CCC(C)(C)c2cc(-c3ccc(N4c5ccc(-c6ccccc6)cc5B5c6ccc(-n7c8ccccc8c8cc(-c9ccccc9)ccc87)cc6N(c6ccc(-c7ccc8c(c7)C(C)(C)CCC8(C)C)cc6)c6cc(-n7c8ccccc8c8cc(-c9ccccc9)ccc87)cc4c65)cc3)ccc21 ; CC(C)(C)c1ccc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccccc6c6cc(-c7ccccc7)ccc65)cc(c42)N3c2ccc(-c3ccc4c(c3)C(C)(C)CCC4(C)C)cc2)cc1]

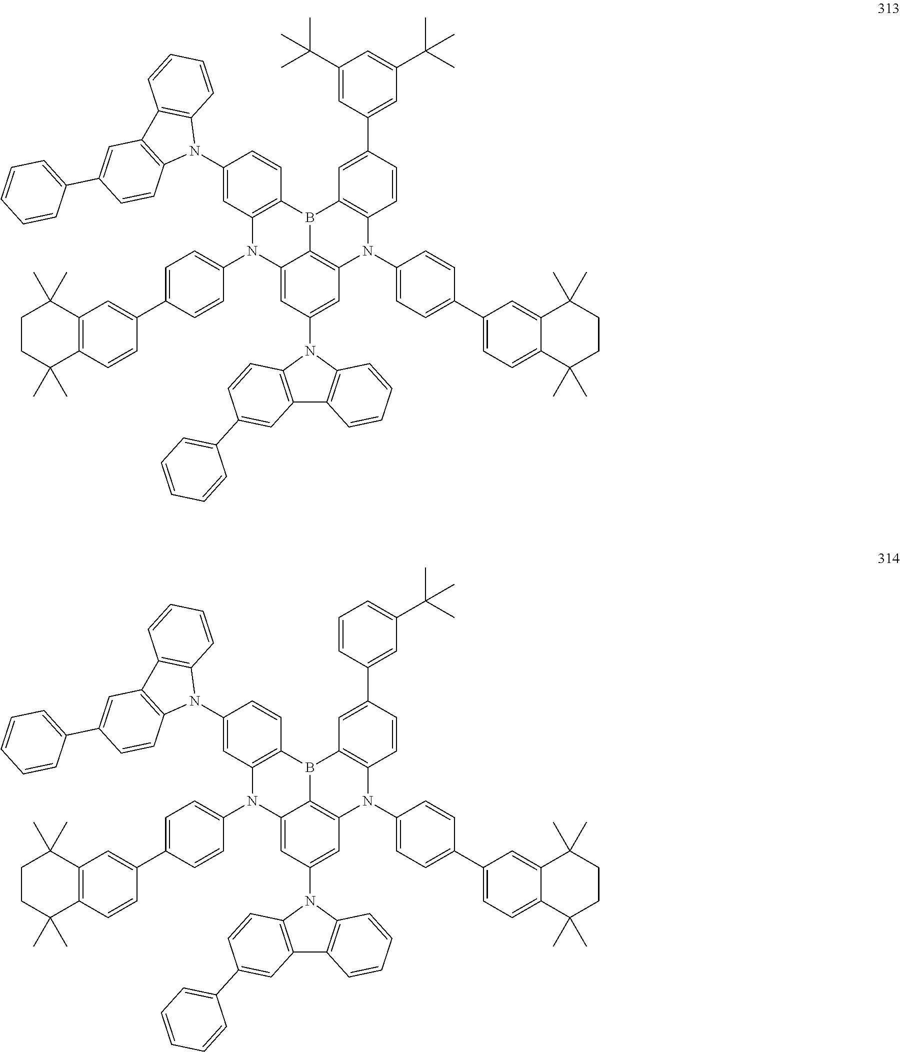 [SMILES: CC(C)(C)c1cc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccccc6c6cc(-c7ccccc7)ccc65)cc(c42)N3c2ccc(-c3ccc4c(c3)C(C)(C)CCC4(C)C)cc2)cc(C(C)(C)C)c1 ; CC(C)(C)c1cccc(-c2ccc3c(c2)B2c4ccc(-n5c6ccccc6c6cc(-c7ccccc7)ccc65)cc4N(c4ccc(-c5ccc6c(c5)C(C)(C)CCC6(C)C)cc4)c4cc(-n5c6ccccc6c6cc(-c7ccccc7)ccc65)cc(c42)N3c2ccc(-c3ccc4c(c3)C(C)(C)CCC4(C)C)cc2)c1]

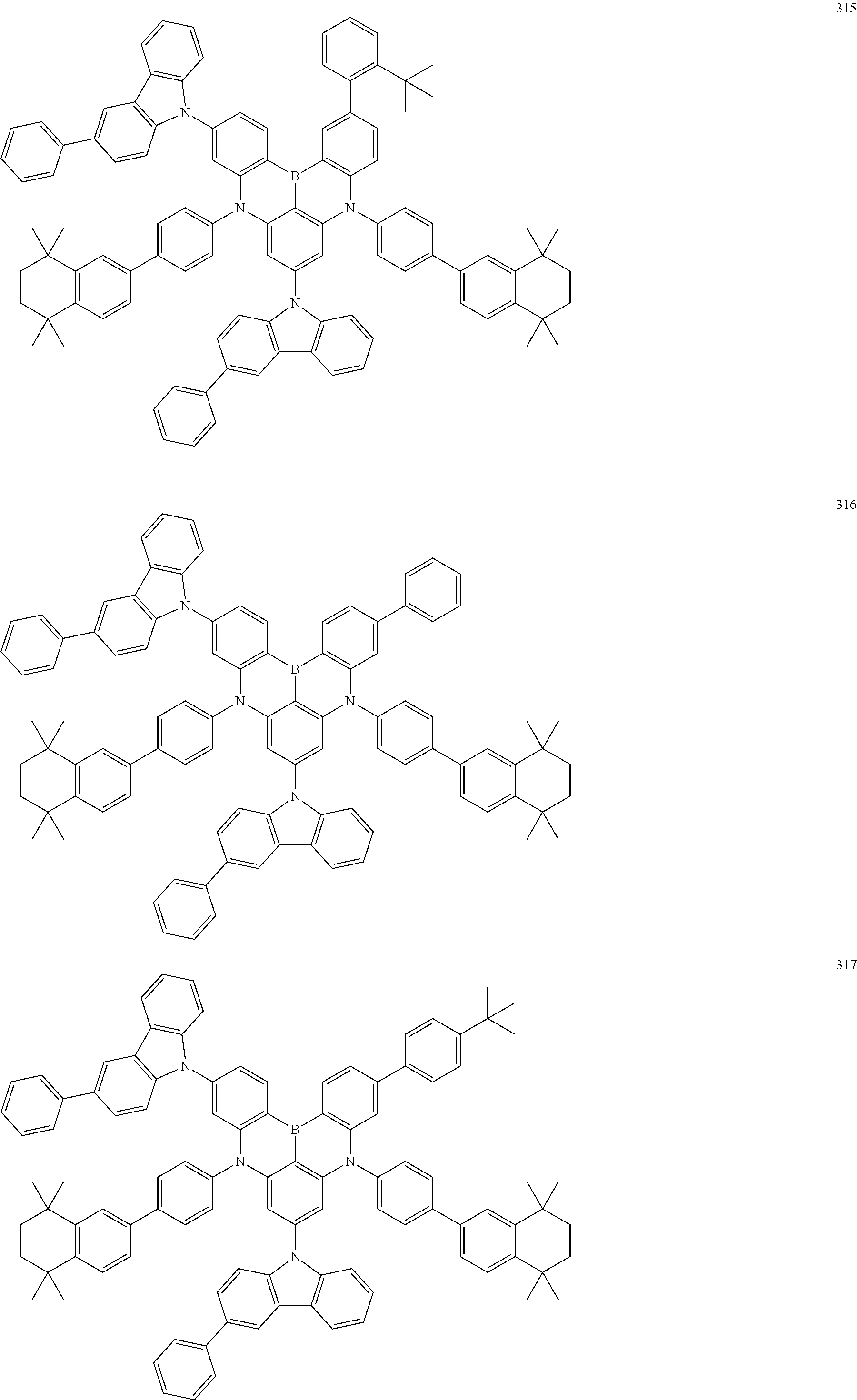

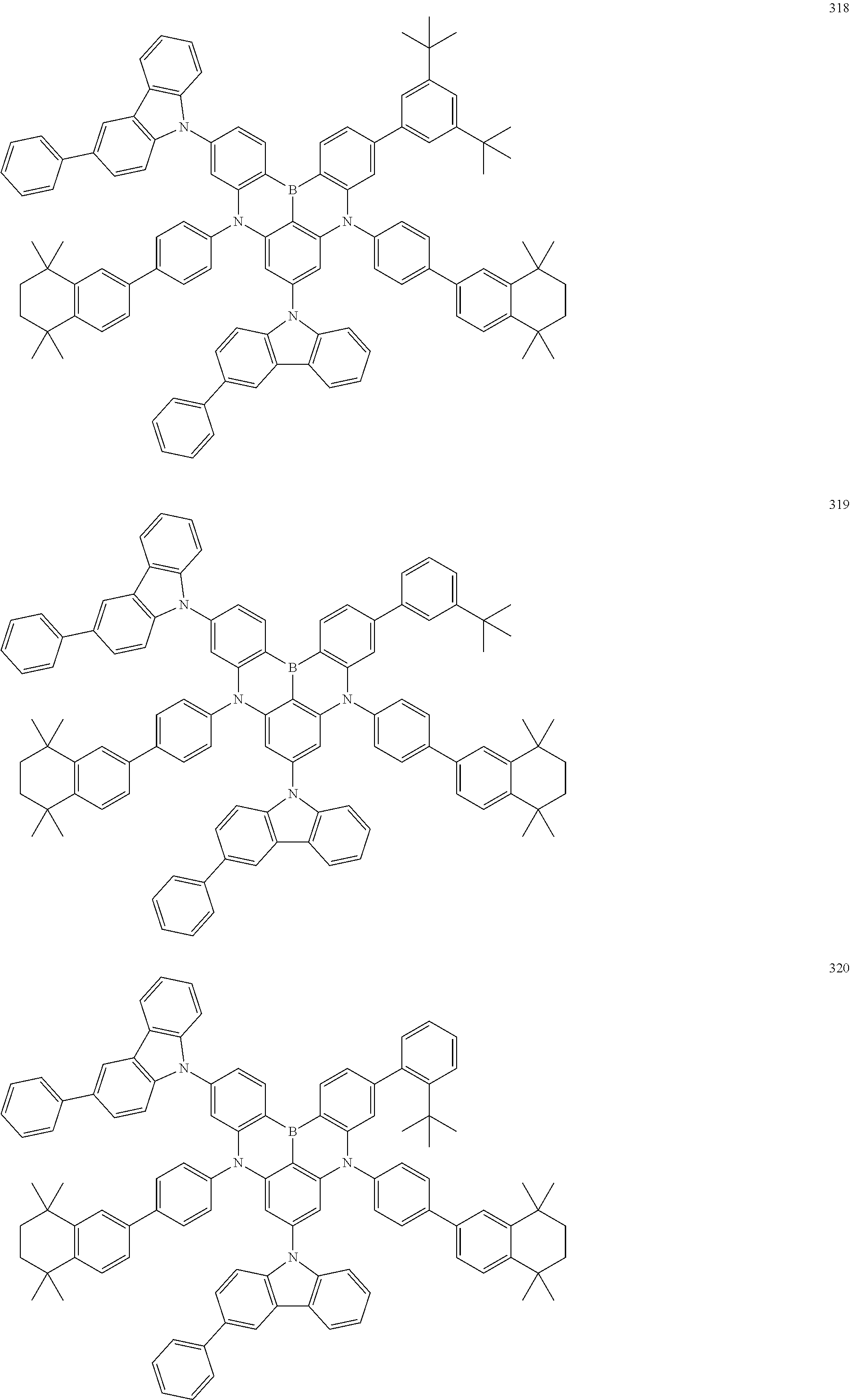

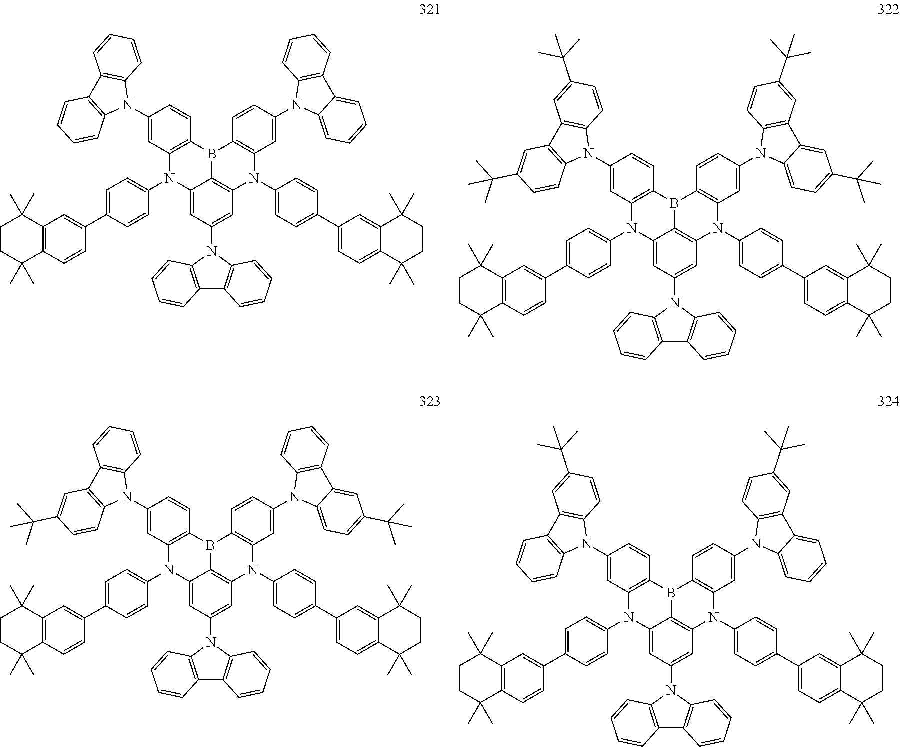

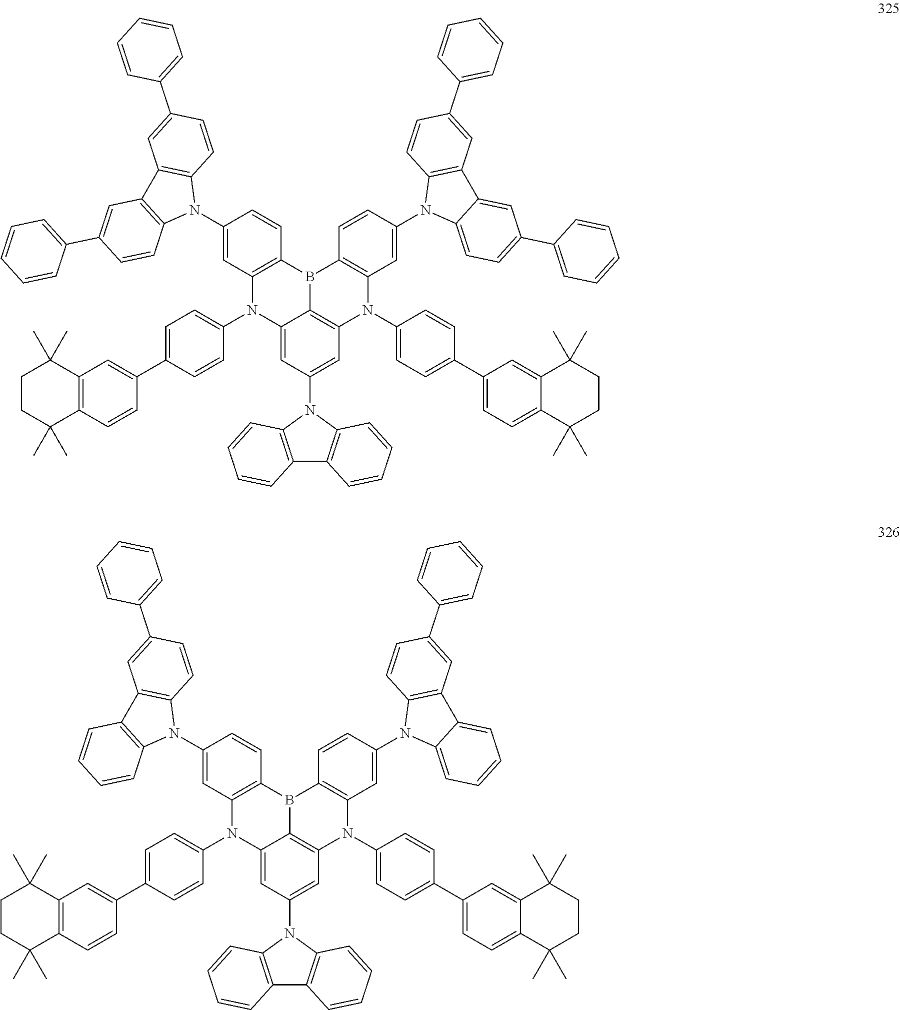 [SMILES: CC1(C)CCC(C)(C)c2cc(-c3ccc(N4c5cc(-n6c7ccc(-c8ccccc8)cc7c7cc(-c8ccccc8)ccc76)ccc5B5c6ccc(-n7c8ccc(-c9ccccc9)cc8c8cc(-c9ccccc9)ccc87)cc6N(c6ccc(-c7ccc8c(c7)C(C)(C)CCC8(C)C)cc6)c6cc(-n7c8ccccc8c8ccccc87)cc4c65)cc3)ccc21 ; CC1(C)CCC(C)(C)c2cc(-c3ccc(N4c5cc(-n6c7ccccc7c7cc(-c8ccccc8)ccc76)ccc5B5c6ccc(-n7c8ccccc8c8cc(-c9ccccc9)ccc87)cc6N(c6ccc(-c7ccc8c(c7)C(C)(C)CCC8(C)C)cc6)c6cc(-n7c8ccccc8c8ccccc87)cc4c65)cc3)ccc21]

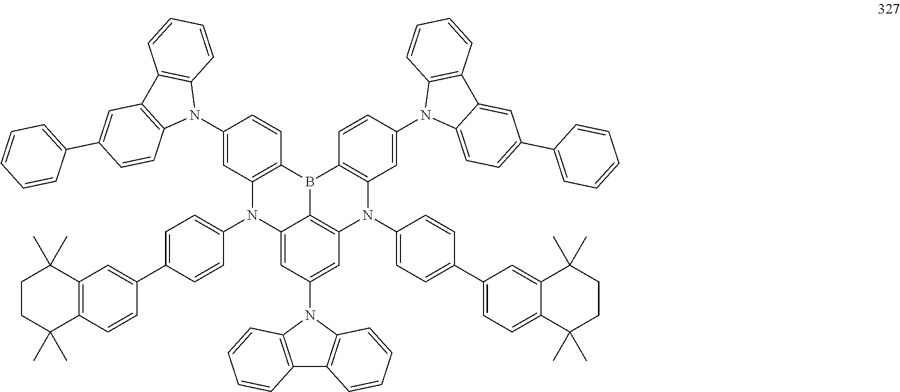 [SMILES: CC1(C)CCC(C)(C)c2cc(-c3ccc(N4c5cc(-n6c7ccccc7c7cc(-c8ccccc8)ccc76)ccc5B5c6ccc(-n7c8ccccc8c8cc(-c9ccccc9)ccc87)cc6N(c6ccc(-c7ccc8c(c7)C(C)(C)CCC8(C)C)cc6)c6cc(-n7c8ccccc8c8ccccc87)cc4c65)cc3)ccc21]

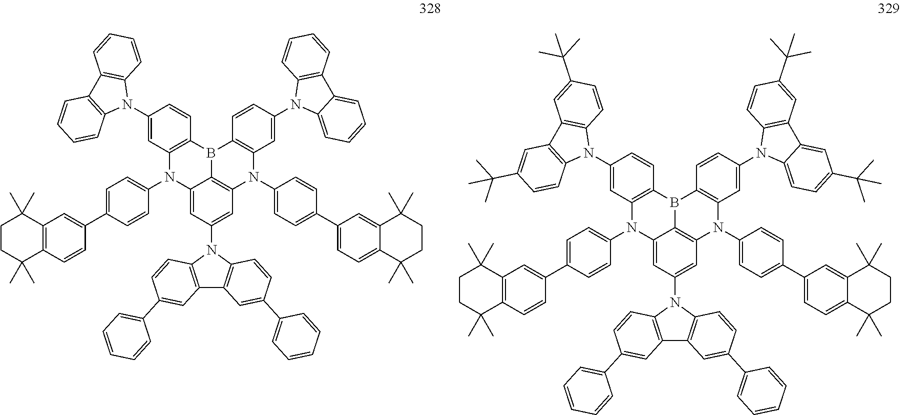 [SMILES: CC1(C)CCC(C)(C)c2cc(-c3ccc(N4c5cc(-n6c7ccccc7c7ccccc76)ccc5B5c6ccc(-n7c8ccccc8c8ccccc87)cc6N(c6ccc(-c7ccc8c(c7)C(C)(C)CCC8(C)C)cc6)c6cc(-n7c8ccc(-c9ccccc9)cc8c8cc(-c9ccccc9)ccc87)cc4c65)cc3)ccc21 ; CC(C)(C)c1ccc2c(c1)c1cc(C(C)(C)C)ccc1n2-c1ccc2c(c1)N(c1ccc(-c3ccc4c(c3)C(C)(C)CCC4(C)C)cc1)c1cc(-n3c4ccc(-c5ccccc5)cc4c4cc(-c5ccccc5)ccc43)cc3c1B2c1ccc(-n2c4ccc(C(C)(C)C)cc4c4cc(C(C)(C)C)ccc42)cc1N3c1ccc(-c2ccc3c(c2)C(C)(C)CCC3(C)C)cc1]

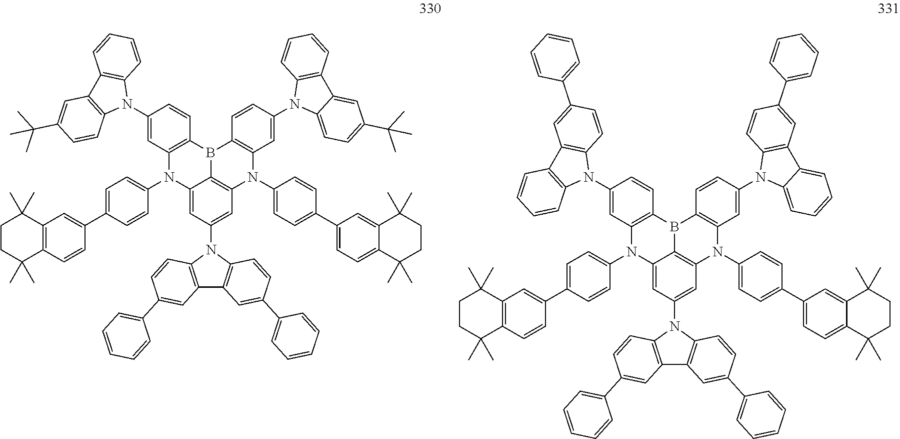 [SMILES: CC(C)(C)c1ccc2c(c1)c1ccccc1n2-c1ccc2c(c1)N(c1ccc(-c3ccc4c(c3)C(C)(C)CCC4(C)C)cc1)c1cc(-n3c4ccc(-c5ccccc5)cc4c4cc(-c5ccccc5)ccc43)cc3c1B2c1ccc(-n2c4ccccc4c4cc(C(C)(C)C)ccc42)cc1N3c1ccc(-c2ccc3c(c2)C(C)(C)CCC3(C)C)cc1 ; CC1(C)CCC(C)(C)c2cc(-c3ccc(N4c5cc(-n6c7ccccc7c7cc(-c8ccccc8)ccc76)ccc5B5c6ccc(-n7c8ccccc8c8cc(-c9ccccc9)ccc87)cc6N(c6ccc(-c7ccc8c(c7)C(C)(C)CCC8(C)C)cc6)c6cc(-n7c8ccc(-c9ccccc9)cc8c8cc(-c9ccccc9)ccc87)cc4c65)cc3)ccc21]

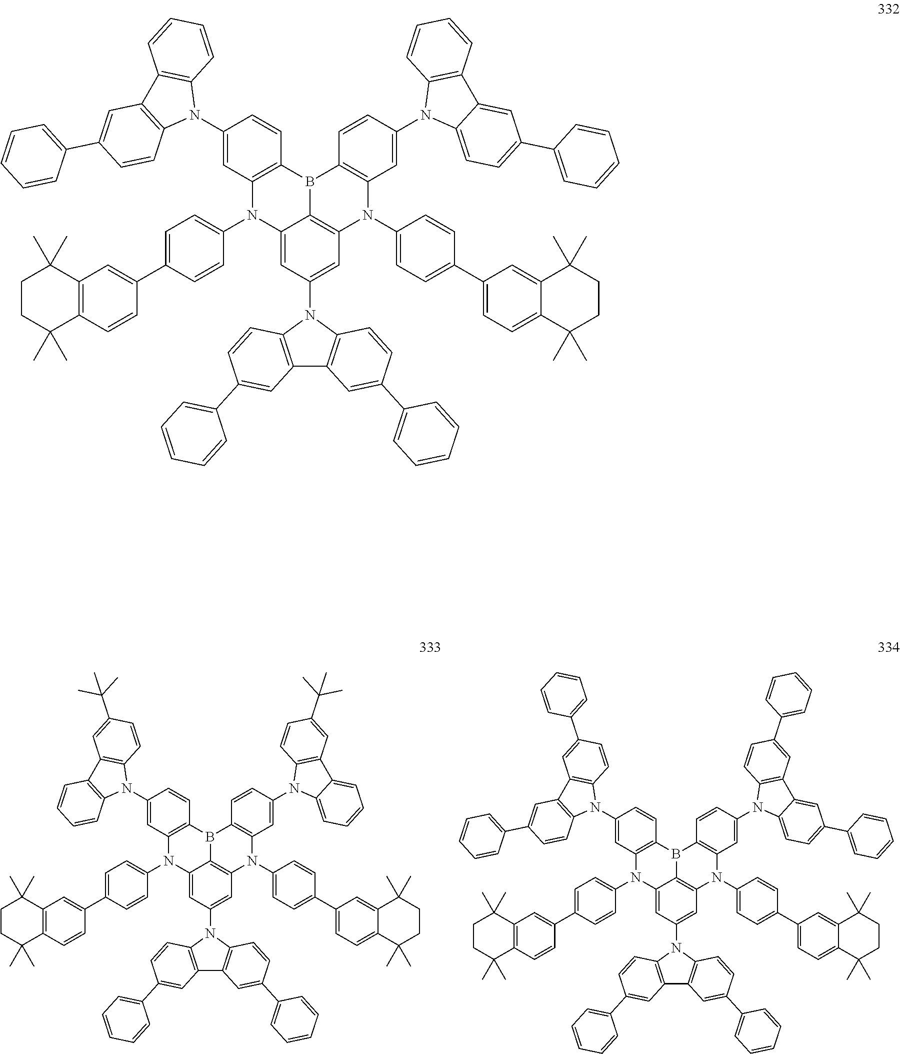

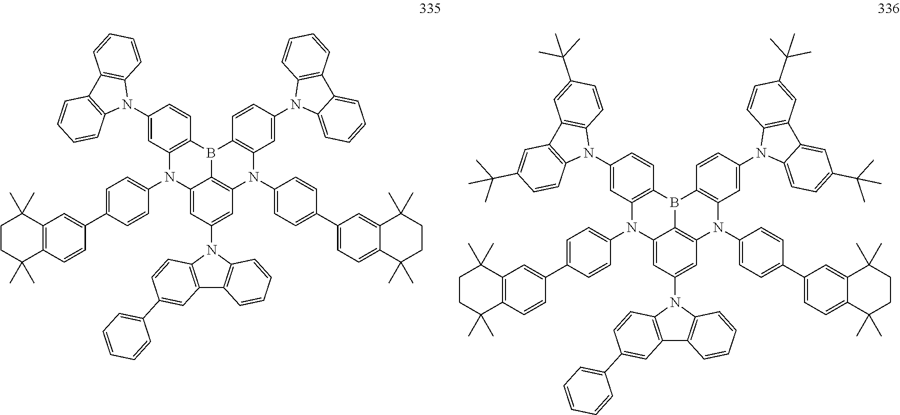 [SMILES: CC1(C)CCC(C)(C)c2cc(-c3ccc(N4c5cc(-n6c7ccccc7c7ccccc76)ccc5B5c6ccc(-n7c8ccccc8c8ccccc87)cc6N(c6ccc(-c7ccc8c(c7)C(C)(C)CCC8(C)C)cc6)c6cc(-n7c8ccccc8c8cc(-c9ccccc9)ccc87)cc4c65)cc3)ccc21 ; CC(C)(C)c1ccc2c(c1)c1cc(C(C)(C)C)ccc1n2-c1ccc2c(c1)N(c1ccc(-c3ccc4c(c3)C(C)(C)CCC4(C)C)cc1)c1cc(-n3c4ccccc4c4cc(-c5ccccc5)ccc43)cc3c1B2c1ccc(-n2c4ccc(C(C)(C)C)cc4c4cc(C(C)(C)C)ccc42)cc1N3c1ccc(-c2ccc3c(c2)C(C)(C)CCC3(C)C)cc1]

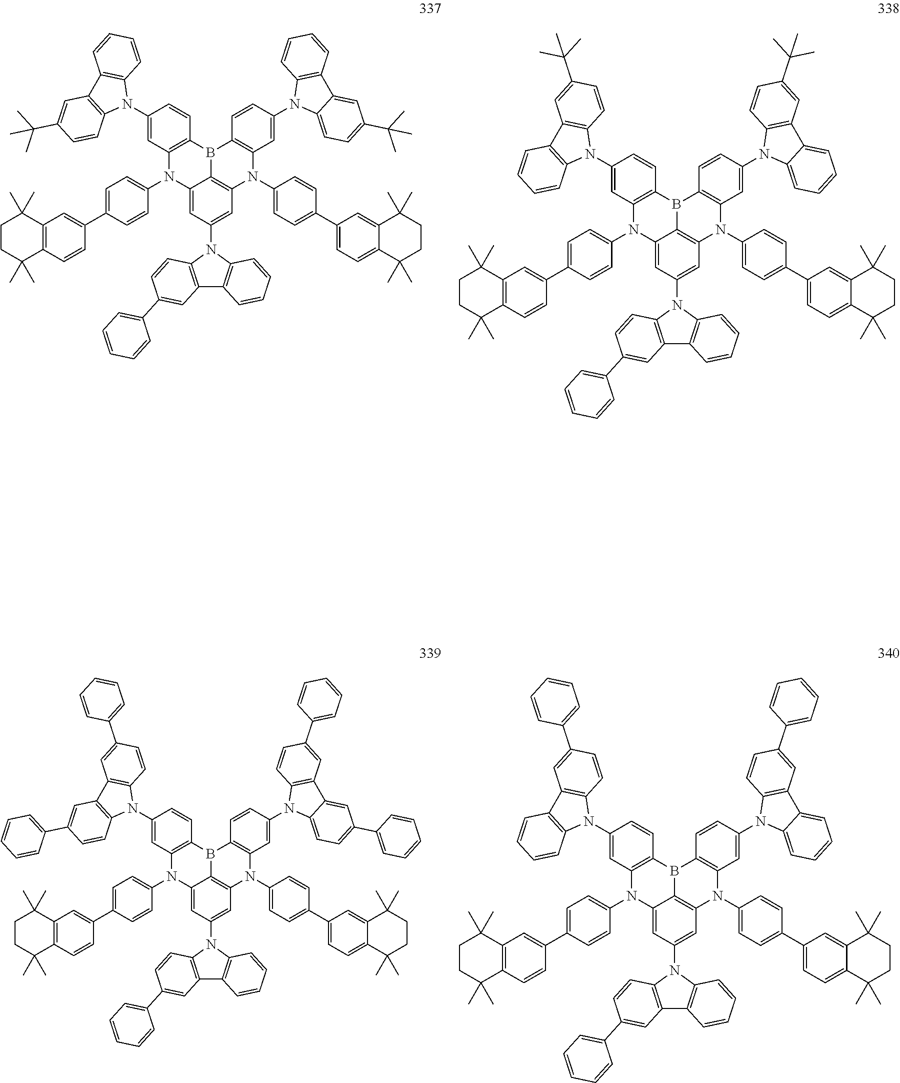

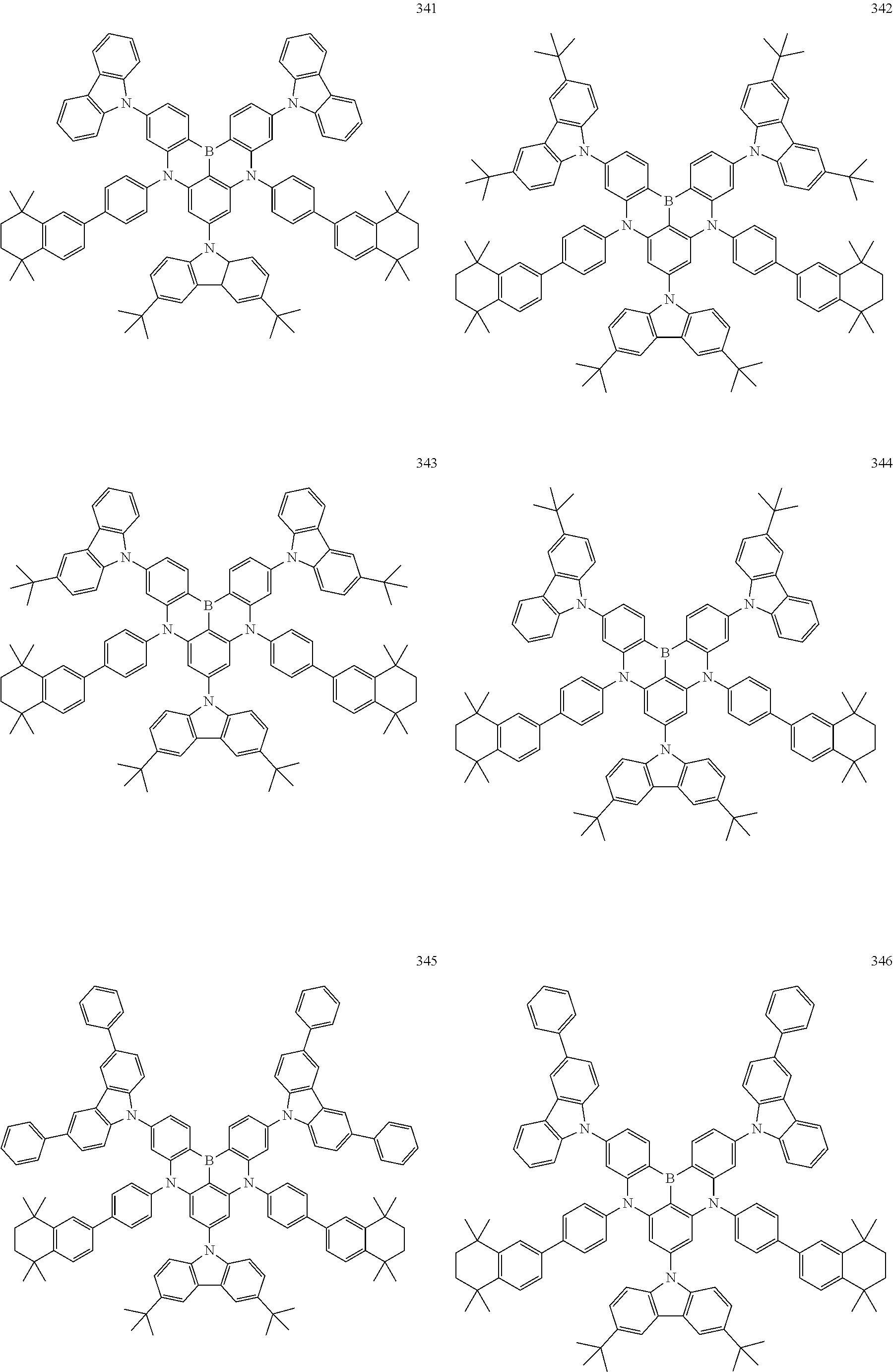

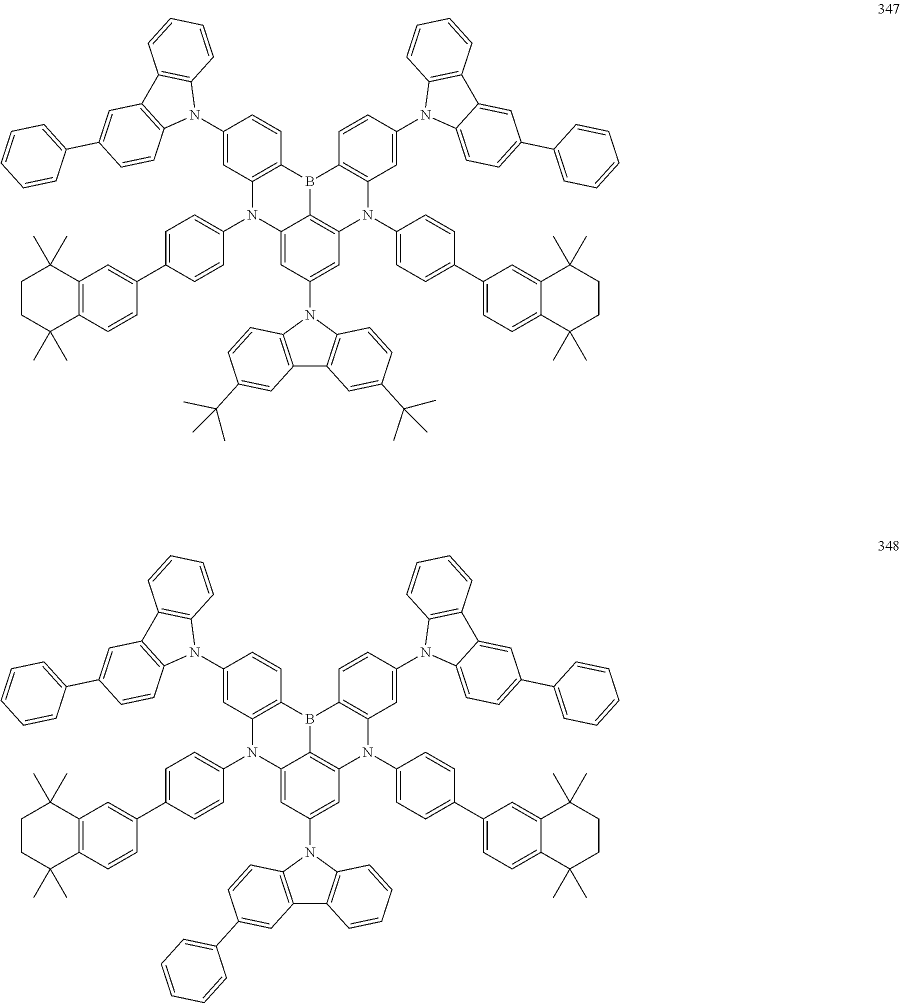 [SMILES: CC(C)(C)c1ccc2c(c1)c1cc(C(C)(C)C)ccc1n2-c1cc2c3c(c1)N(c1ccc(-c4ccc5c(c4)C(C)(C)CCC5(C)C)cc1)c1cc(-n4c5ccccc5c5cc(-c6ccccc6)ccc54)ccc1B3c1ccc(-n3c4ccccc4c4cc(-c5ccccc5)ccc43)cc1N2c1ccc(-c2ccc3c(c2)C(C)(C)CCC3(C)C)cc1 ; CC1(C)CCC(C)(C)c2cc(-c3ccc(N4c5cc(-n6c7ccccc7c7cc(-c8ccccc8)ccc76)ccc5B5c6ccc(-n7c8ccccc8c8cc(-c9ccccc9)ccc87)cc6N(c6ccc(-c7ccc8c(c7)C(C)(C)CCC8(C)C)cc6)c6cc(-n7c8ccccc8c8cc(-c9ccccc9)ccc87)cc4c65)cc3)ccc21]

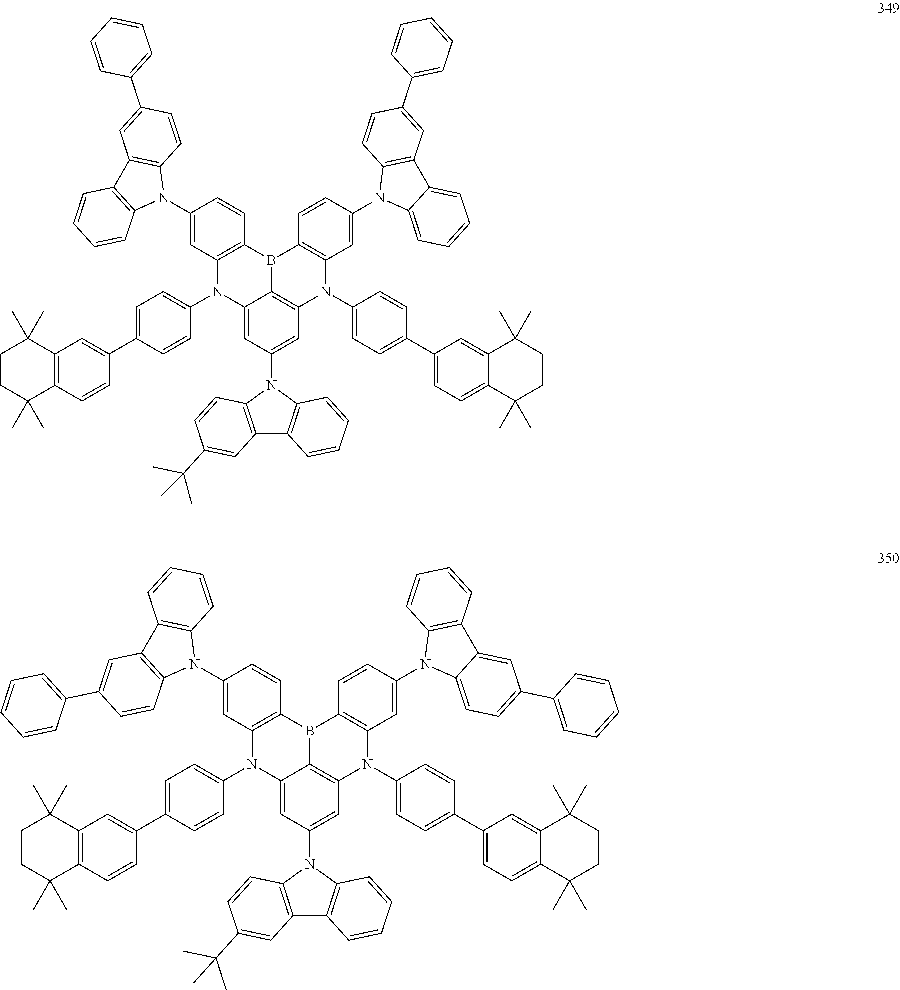 [SMILES: CC(C)(C)c1ccc2c(c1)c1ccccc1n2-c1cc2c3c(c1)N(c1ccc(-c4ccc5c(c4)C(C)(C)CCC5(C)C)cc1)c1cc(-n4c5ccccc5c5cc(-c6ccccc6)ccc54)ccc1B3c1ccc(-n3c4ccccc4c4cc(-c5ccccc5)ccc43)cc1N2c1ccc(-c2ccc3c(c2)C(C)(C)CCC3(C)C)cc1 ; CC(C)(C)c1ccc2c(c1)c1ccccc1n2-c1cc2c3c(c1)N(c1ccc(-c4ccc5c(c4)C(C)(C)CCC5(C)C)cc1)c1cc(-n4c5ccccc5c5cc(-c6ccccc6)ccc54)ccc1B3c1ccc(-n3c4ccccc4c4cc(-c5ccccc5)ccc43)cc1N2c1ccc(-c2ccc3c(c2)C(C)(C)CCC3(C)C)cc1]

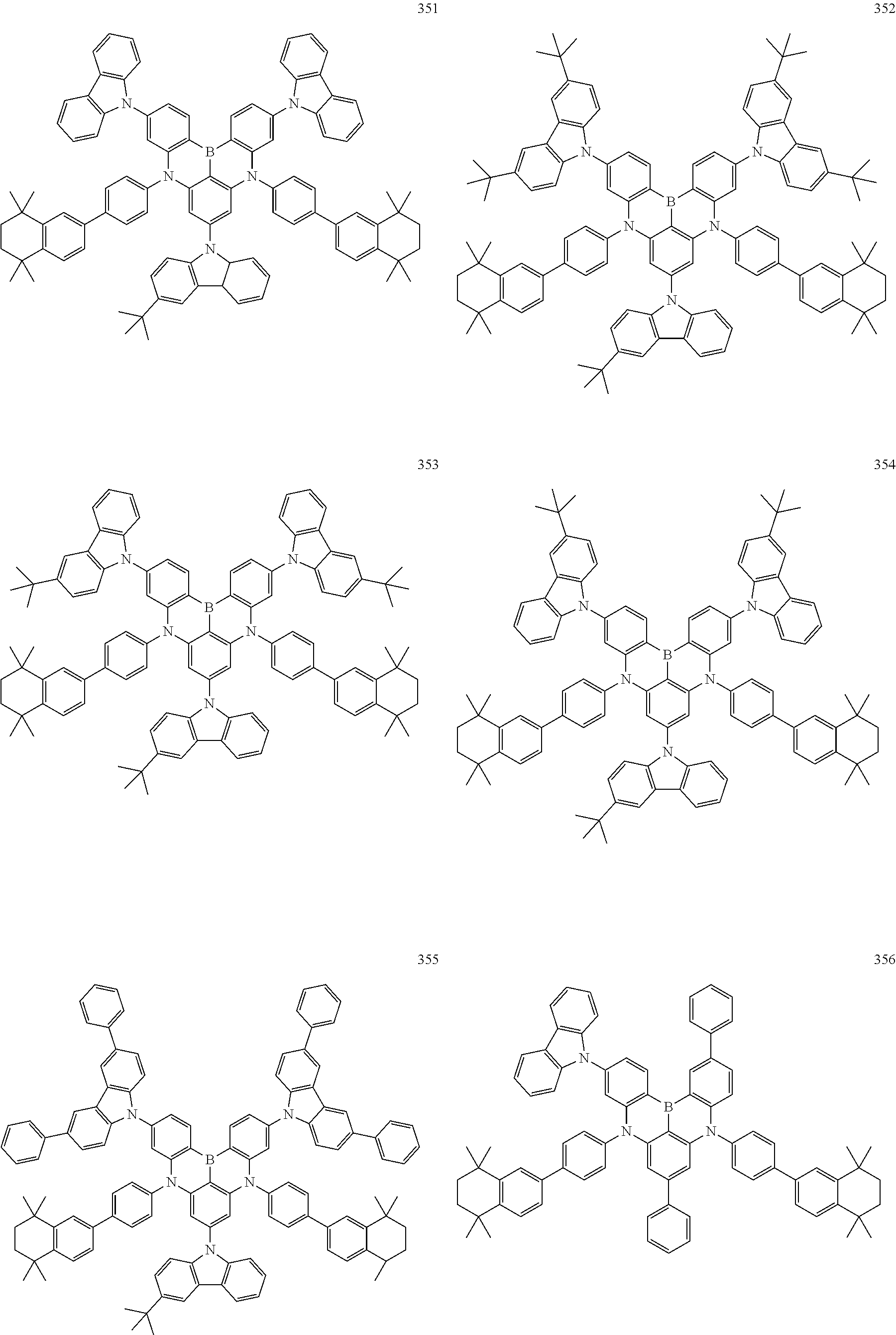

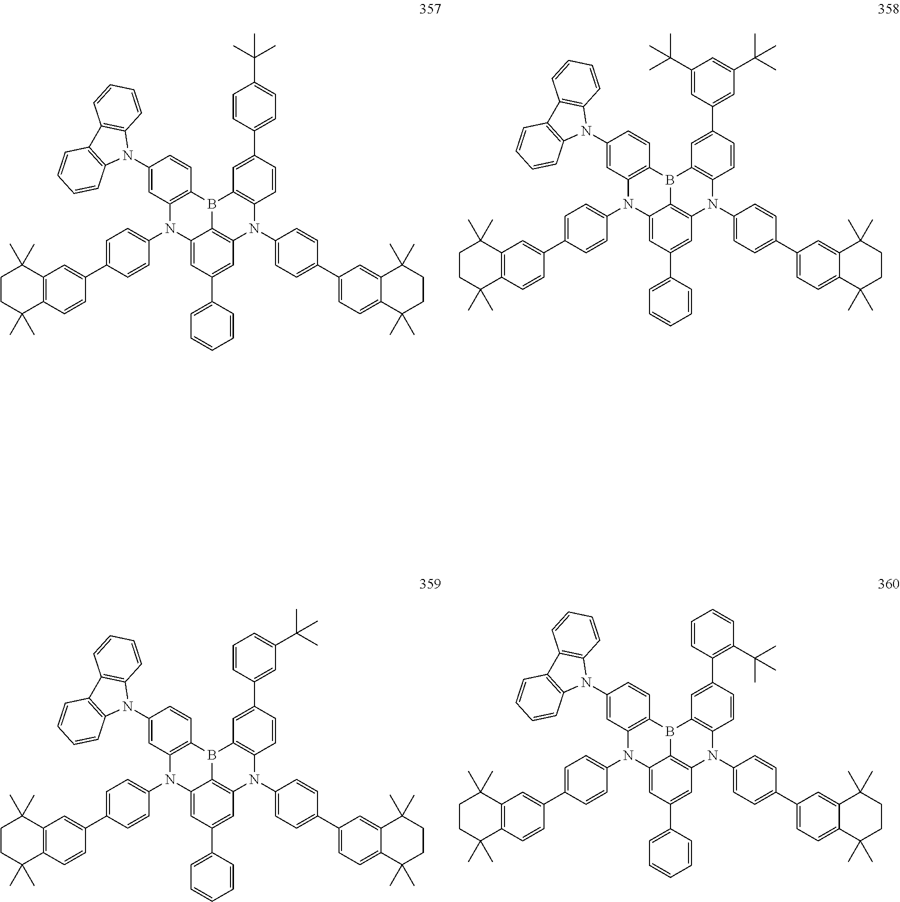

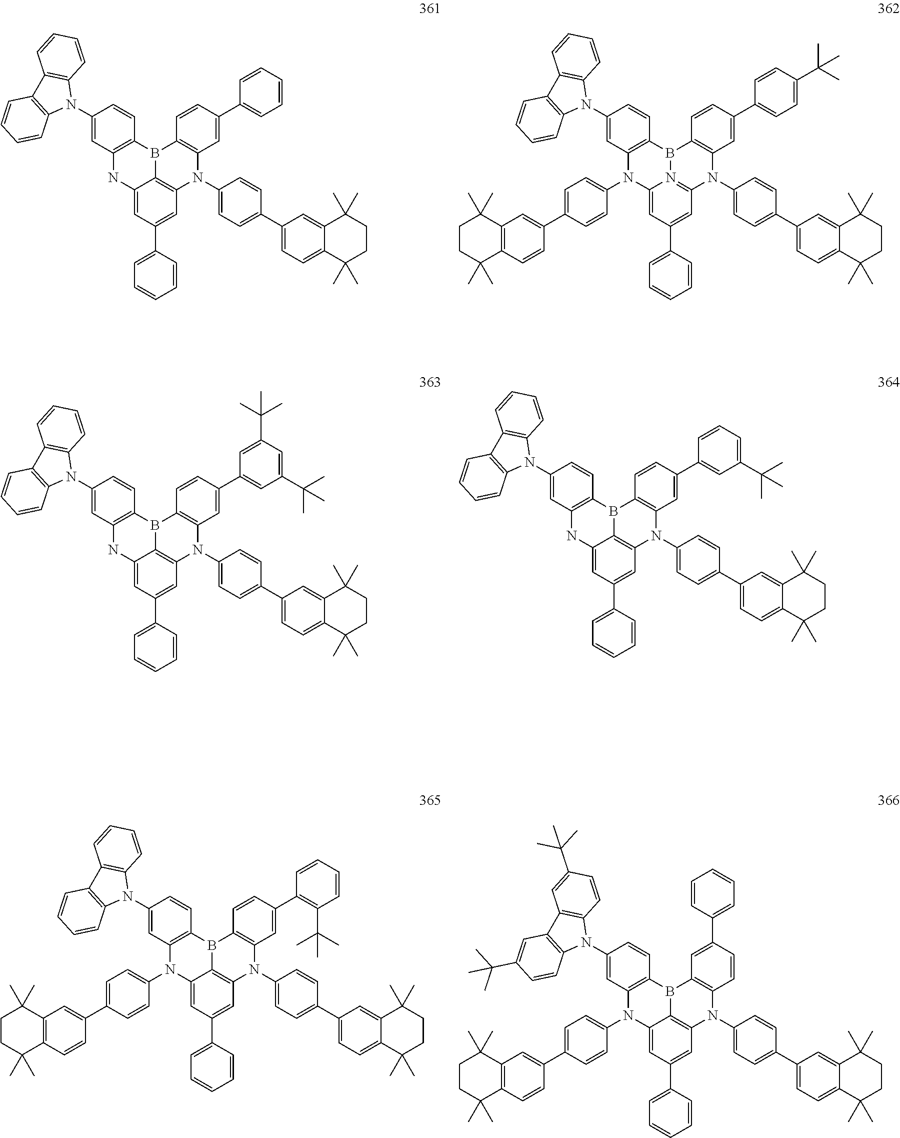

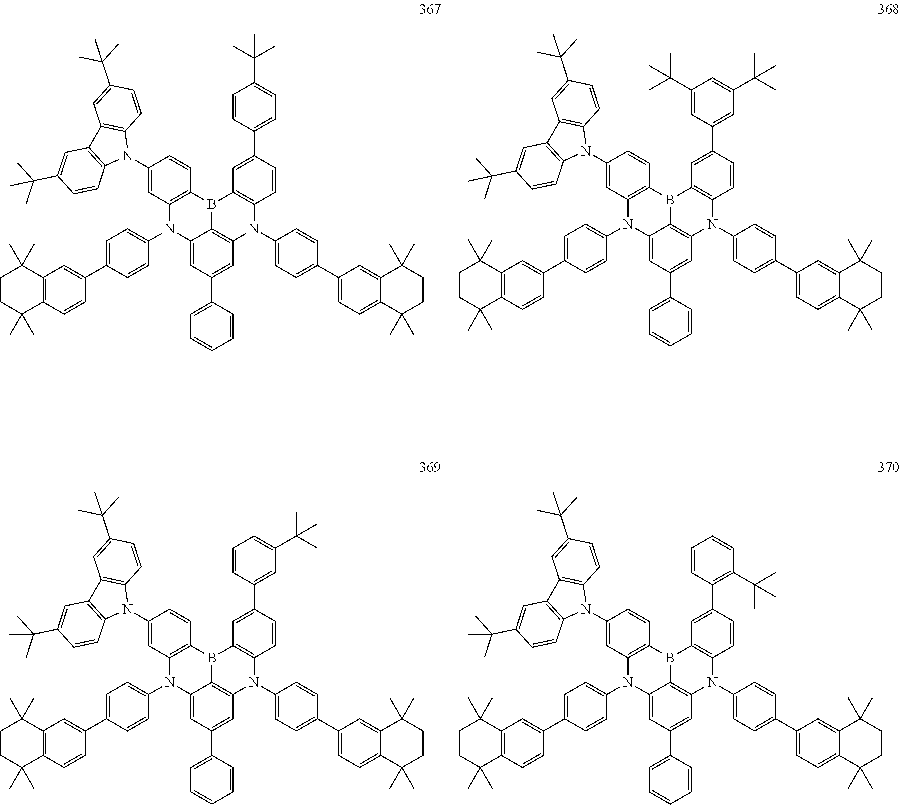

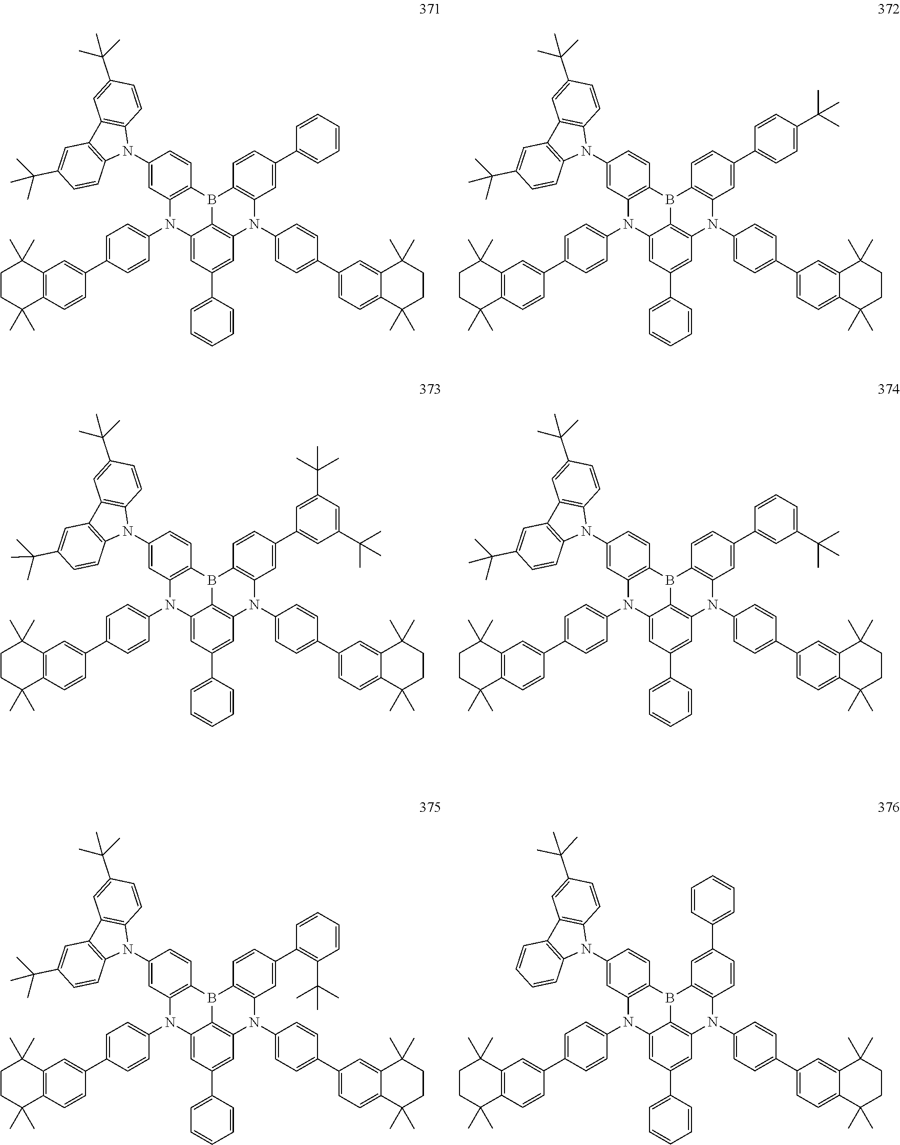

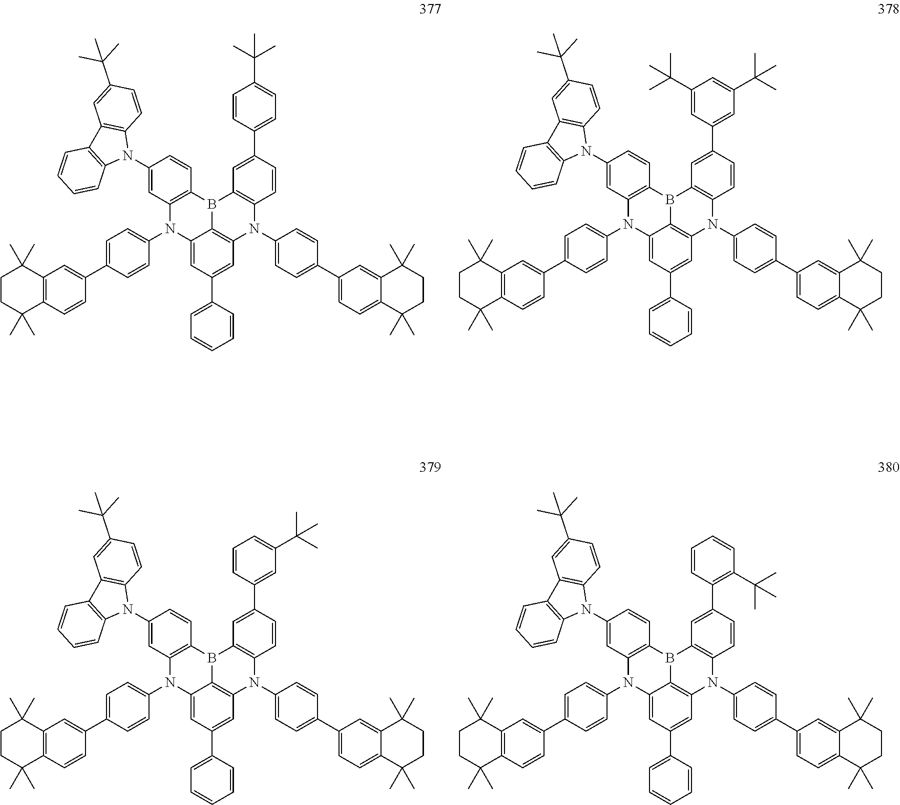

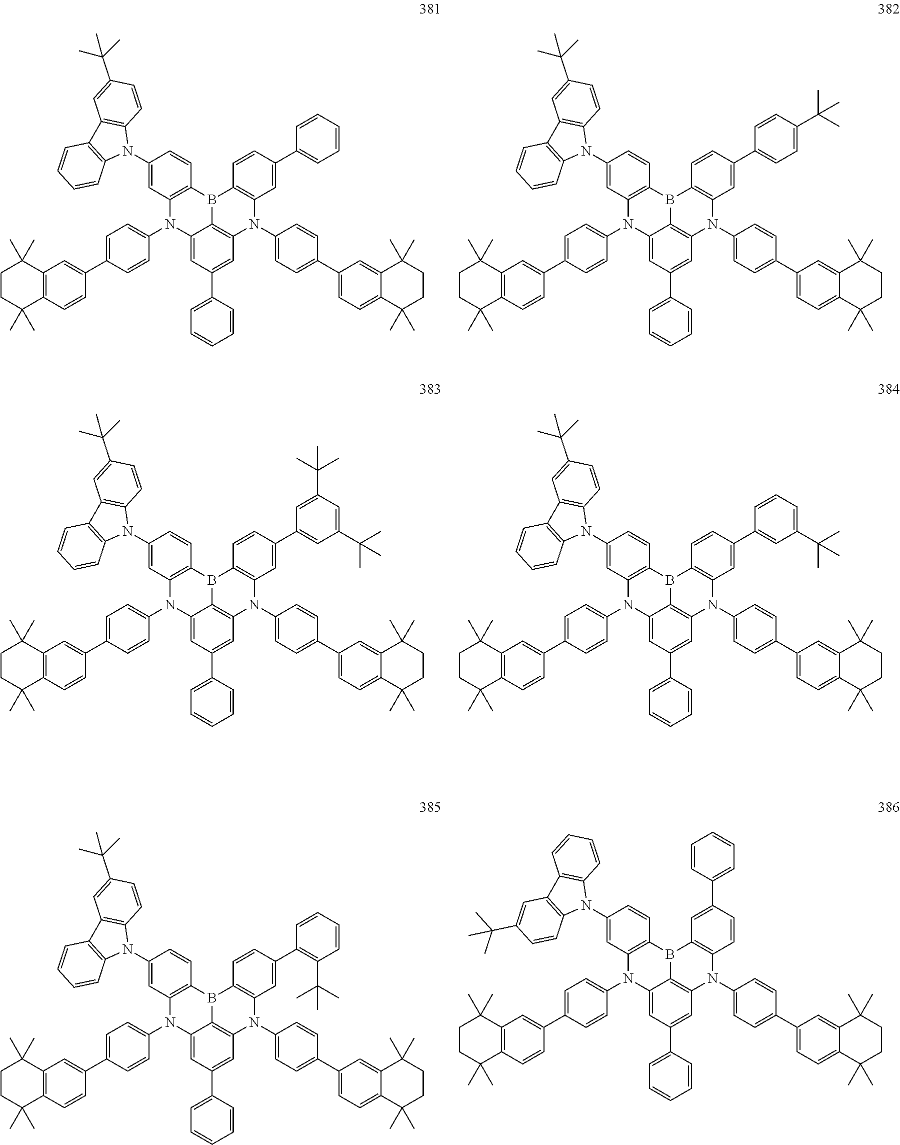

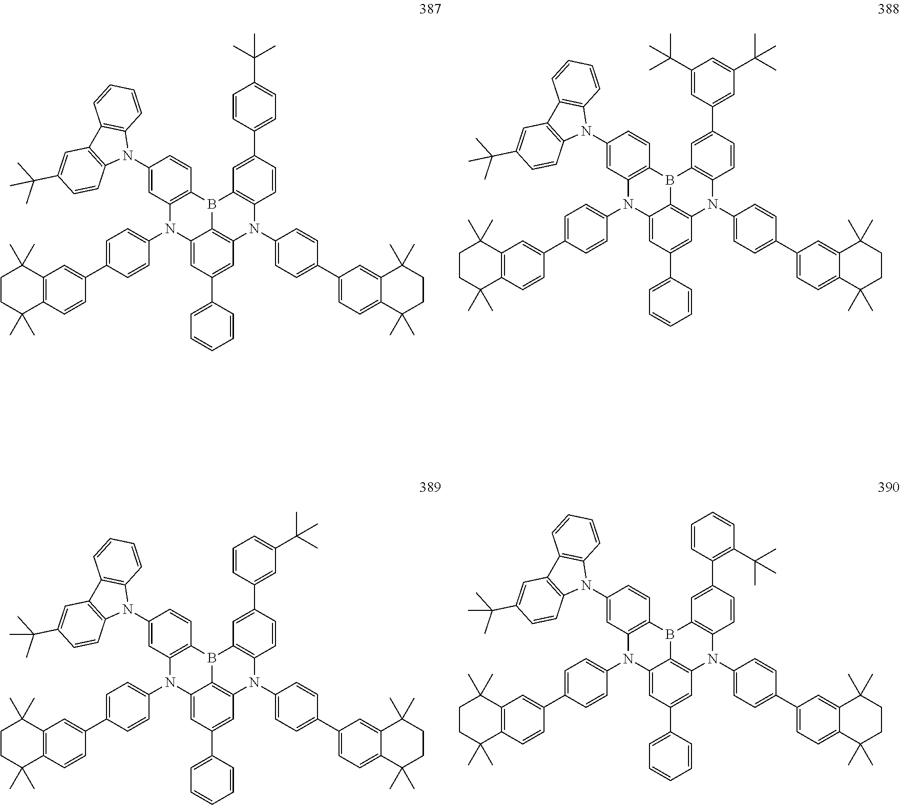

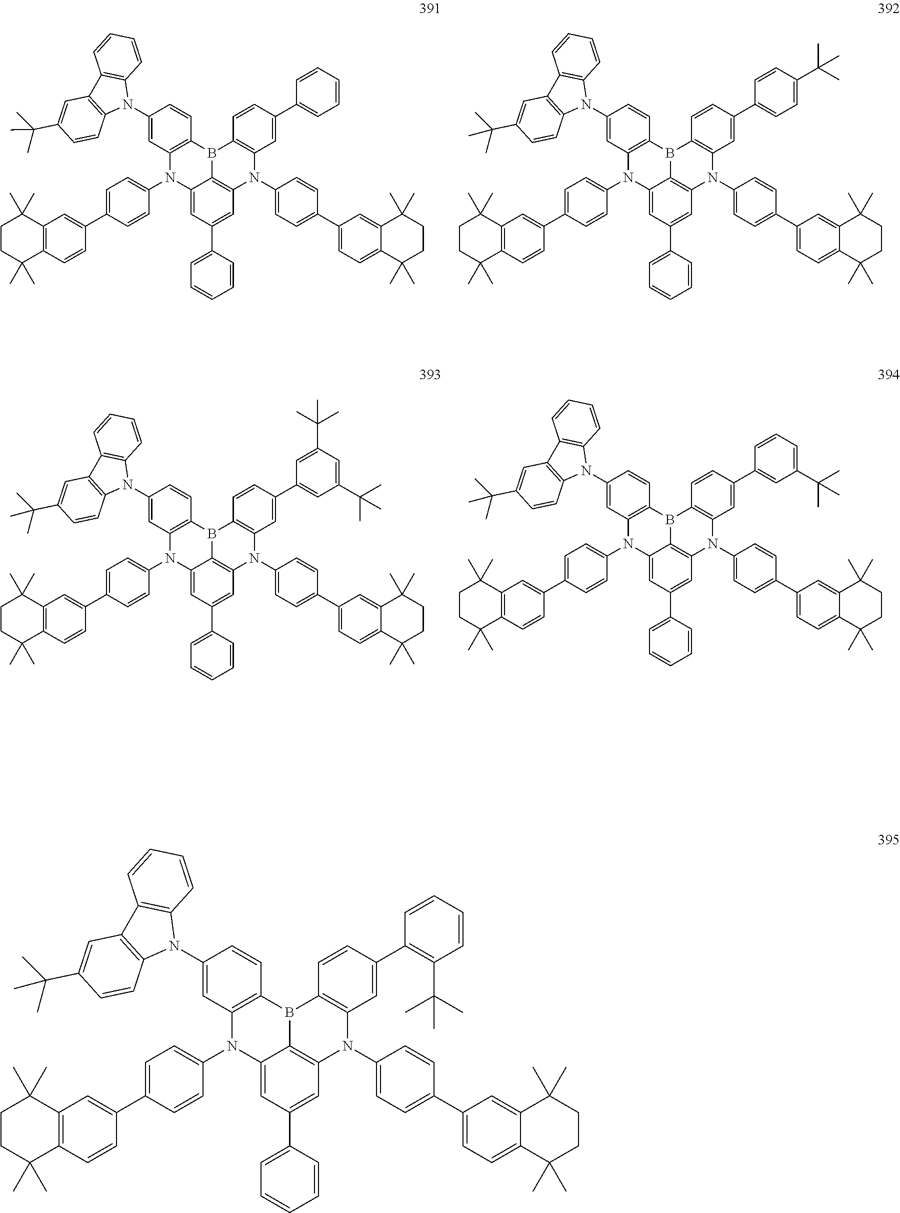

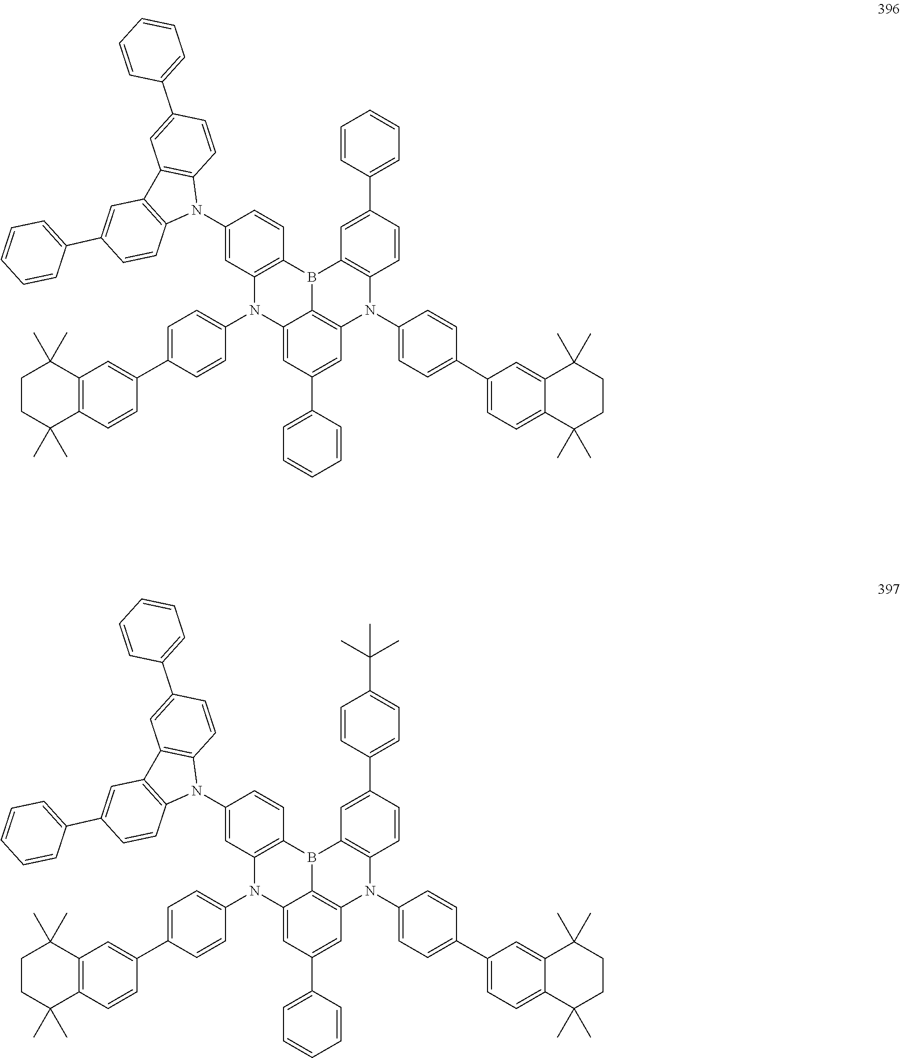 [SMILES: CC1(C)CCC(C)(C)c2cc(-c3ccc(N4c5ccc(-c6ccccc6)cc5B5c6ccc(-n7c8ccc(-c9ccccc9)cc8c8cc(-c9ccccc9)ccc87)cc6N(c6ccc(-c7ccc8c(c7)C(C)(C)CCC8(C)C)cc6)c6cc(-c7ccccc7)cc4c65)cc3)ccc21 ; CC(C)(C)c1ccc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CCC6(C)C)cc4)c4cc(-c5ccccc5)cc(c42)N3c2ccc(-c3ccc4c(c3)C(C)(C)CCC4(C)C)cc2)cc1]

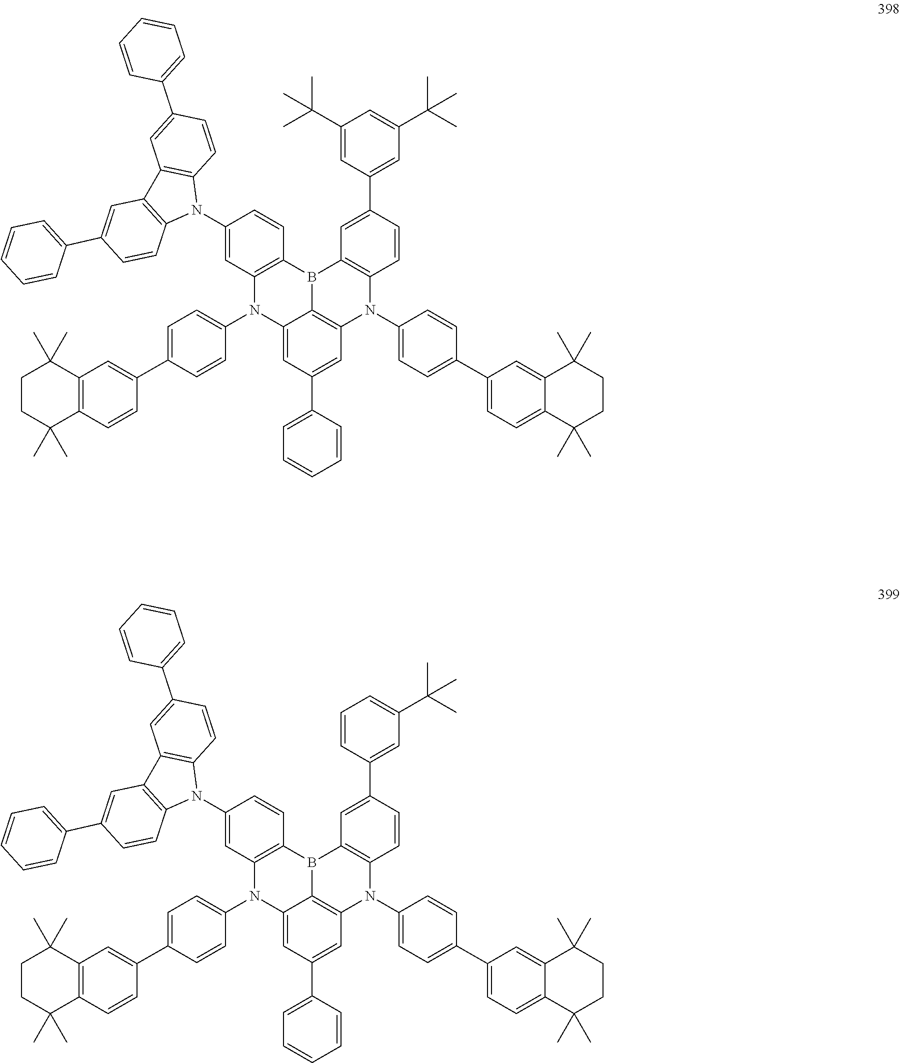 [SMILES: CC(C)(C)c1cc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CCC6(C)C)cc4)c4cc(-c5ccccc5)cc(c42)N3c2ccc(-c3ccc4c(c3)C(C)(C)CCC4(C)C)cc2)cc(C(C)(C)C)c1 ; CC(C)(C)c1cccc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CCC6(C)C)cc4)c4cc(-c5ccccc5)cc(c42)N3c2ccc(-c3ccc4c(c3)C(C)(C)CCC4(C)C)cc2)c1]

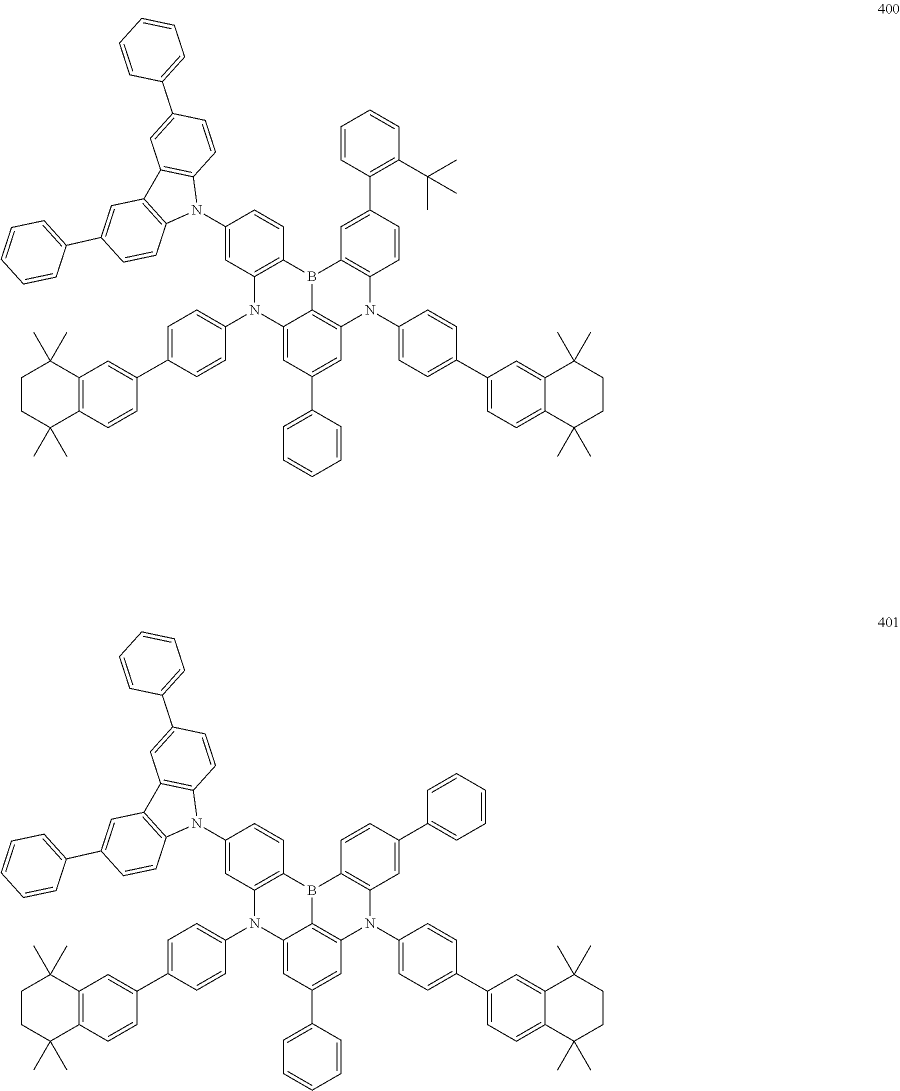 [SMILES: CC(C)(C)c1ccccc1-c1ccc2c(c1)B1c3ccc(-n4c5ccc(-c6ccccc6)cc5c5cc(-c6ccccc6)ccc54)cc3N(c3ccc(-c4ccc5c(c4)C(C)(C)CCC5(C)C)cc3)c3cc(-c4ccccc4)cc(c31)N2c1ccc(-c2ccc3c(c2)C(C)(C)CCC3(C)C)cc1 ; CC1(C)CCC(C)(C)c2cc(-c3ccc(N4c5cc(-c6ccccc6)ccc5B5c6ccc(-n7c8ccc(-c9ccccc9)cc8c8cc(-c9ccccc9)ccc87)cc6N(c6ccc(-c7ccc8c(c7)C(C)(C)CCC8(C)C)cc6)c6cc(-c7ccccc7)cc4c65)cc3)ccc21]

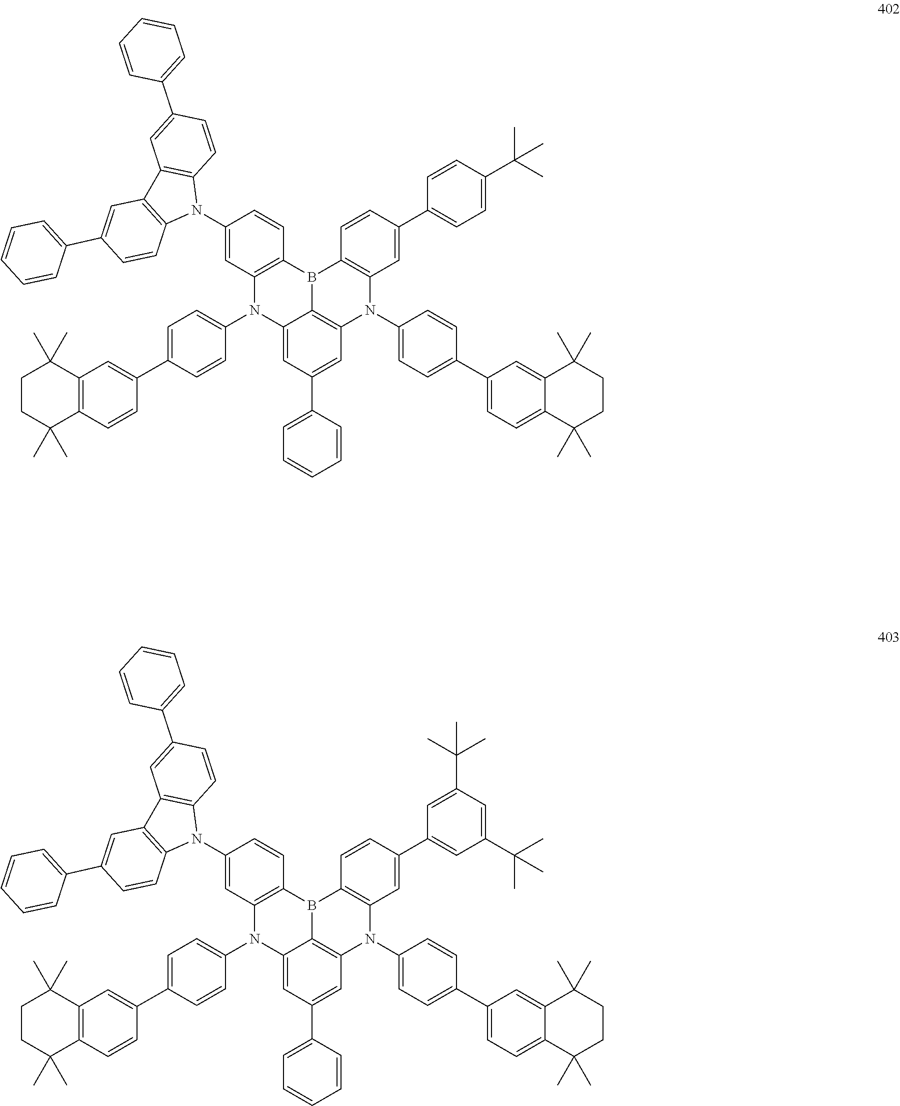 [SMILES: CC(C)(C)c1ccc(-c2ccc3c(c2)N(c2ccc(-c4ccc5c(c4)C(C)(C)CCC5(C)C)cc2)c2cc(-c4ccccc4)cc4c2B3c2ccc(-n3c5ccc(-c6ccccc6)cc5c5cc(-c6ccccc6)ccc53)cc2N4c2ccc(-c3ccc4c(c3)C(C)(C)CCC4(C)C)cc2)cc1 ; CC(C)(C)c1cc(-c2ccc3c(c2)N(c2ccc(-c4ccc5c(c4)C(C)(C)CCC5(C)C)cc2)c2cc(-c4ccccc4)cc4c2B3c2ccc(-n3c5ccc(-c6ccccc6)cc5c5cc(-c6ccccc6)ccc53)cc2N4c2ccc(-c3ccc4c(c3)C(C)(C)CCC4(C)C)cc2)cc(C(C)(C)C)c1]

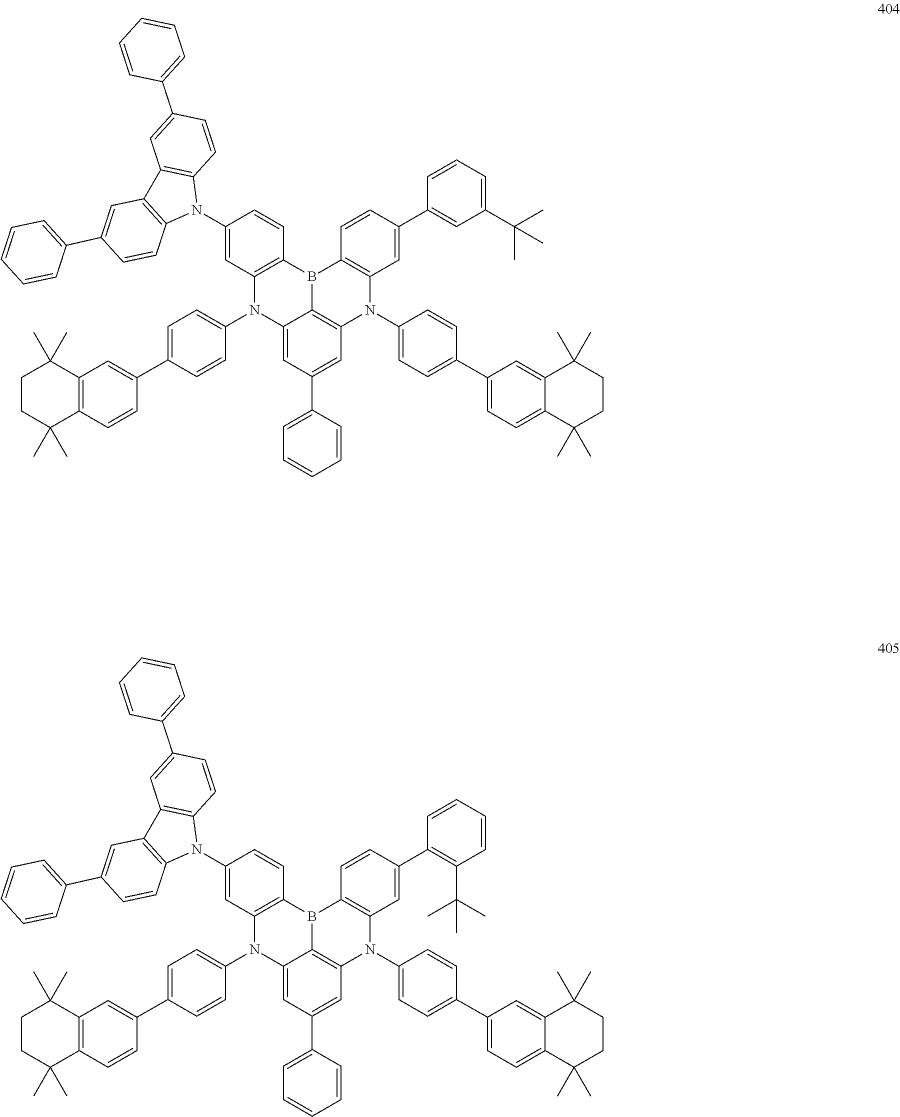 [SMILES: CC(C)(C)c1cccc(-c2ccc3c(c2)N(c2ccc(-c4ccc5c(c4)C(C)(C)CCC5(C)C)cc2)c2cc(-c4ccccc4)cc4c2B3c2ccc(-n3c5ccc(-c6ccccc6)cc5c5cc(-c6ccccc6)ccc53)cc2N4c2ccc(-c3ccc4c(c3)C(C)(C)CCC4(C)C)cc2)c1 ; CC(C)(C)c1ccccc1-c1ccc2c(c1)N(c1ccc(-c3ccc4c(c3)C(C)(C)CCC4(C)C)cc1)c1cc(-c3ccccc3)cc3c1B2c1ccc(-n2c4ccc(-c5ccccc5)cc4c4cc(-c5ccccc5)ccc42)cc1N3c1ccc(-c2ccc3c(c2)C(C)(C)CCC3(C)C)cc1]

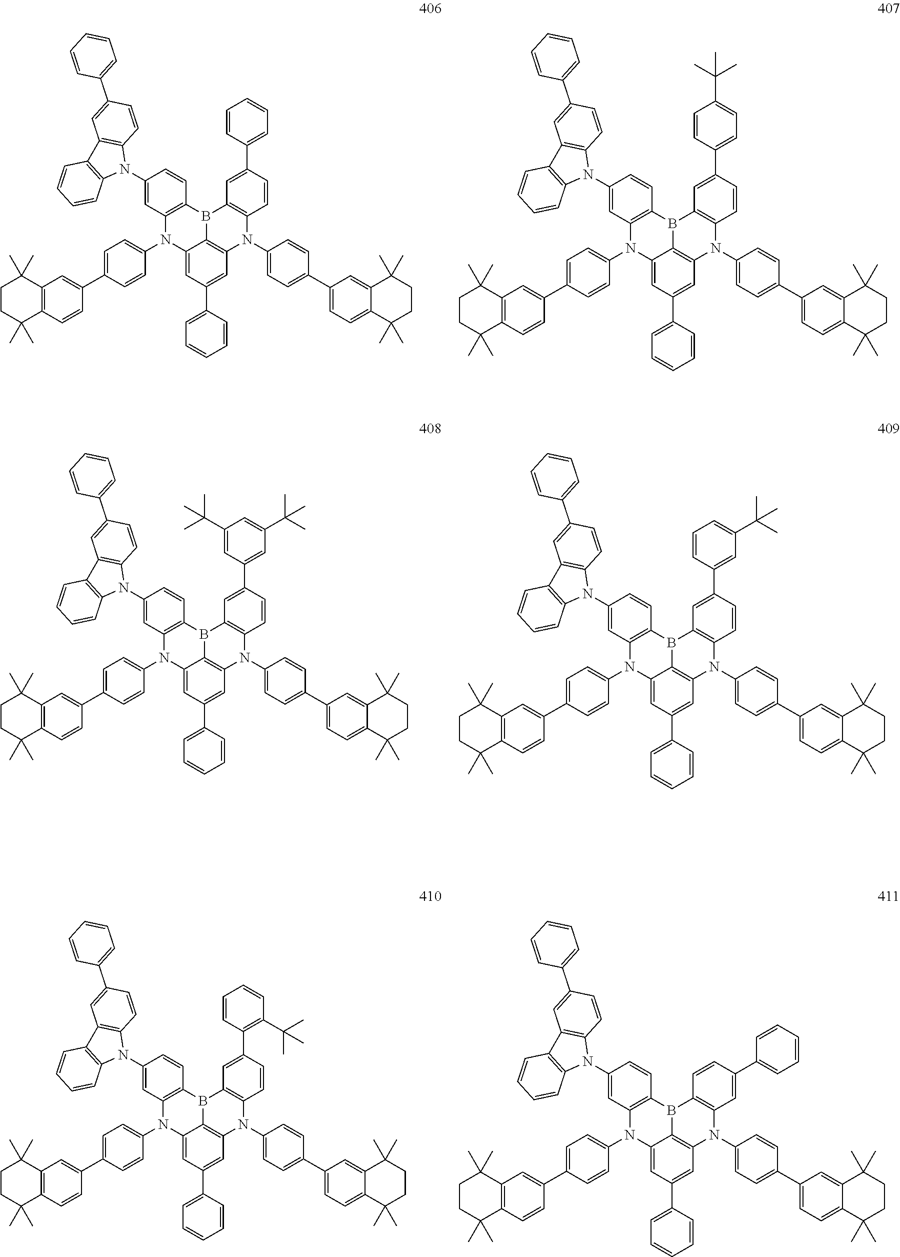

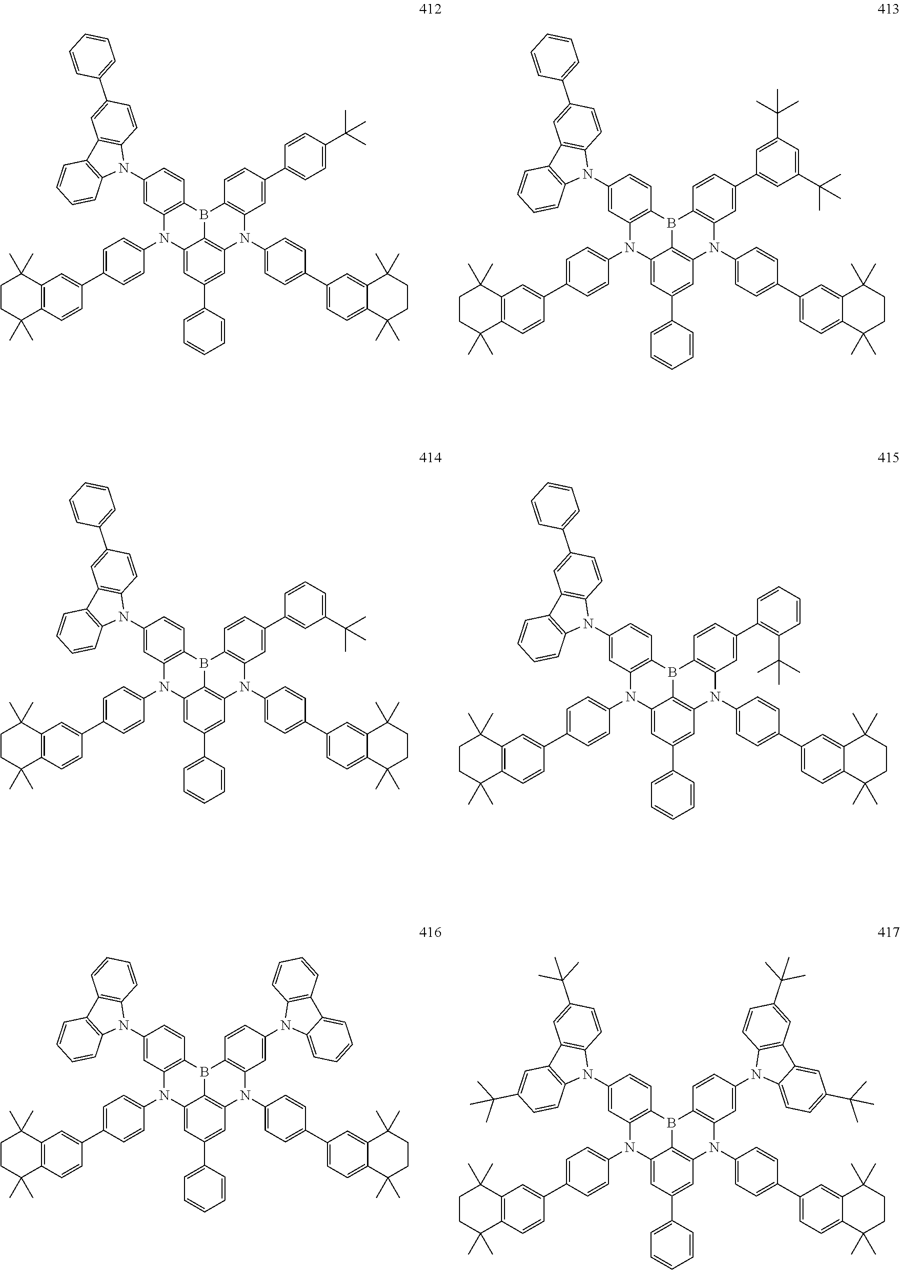

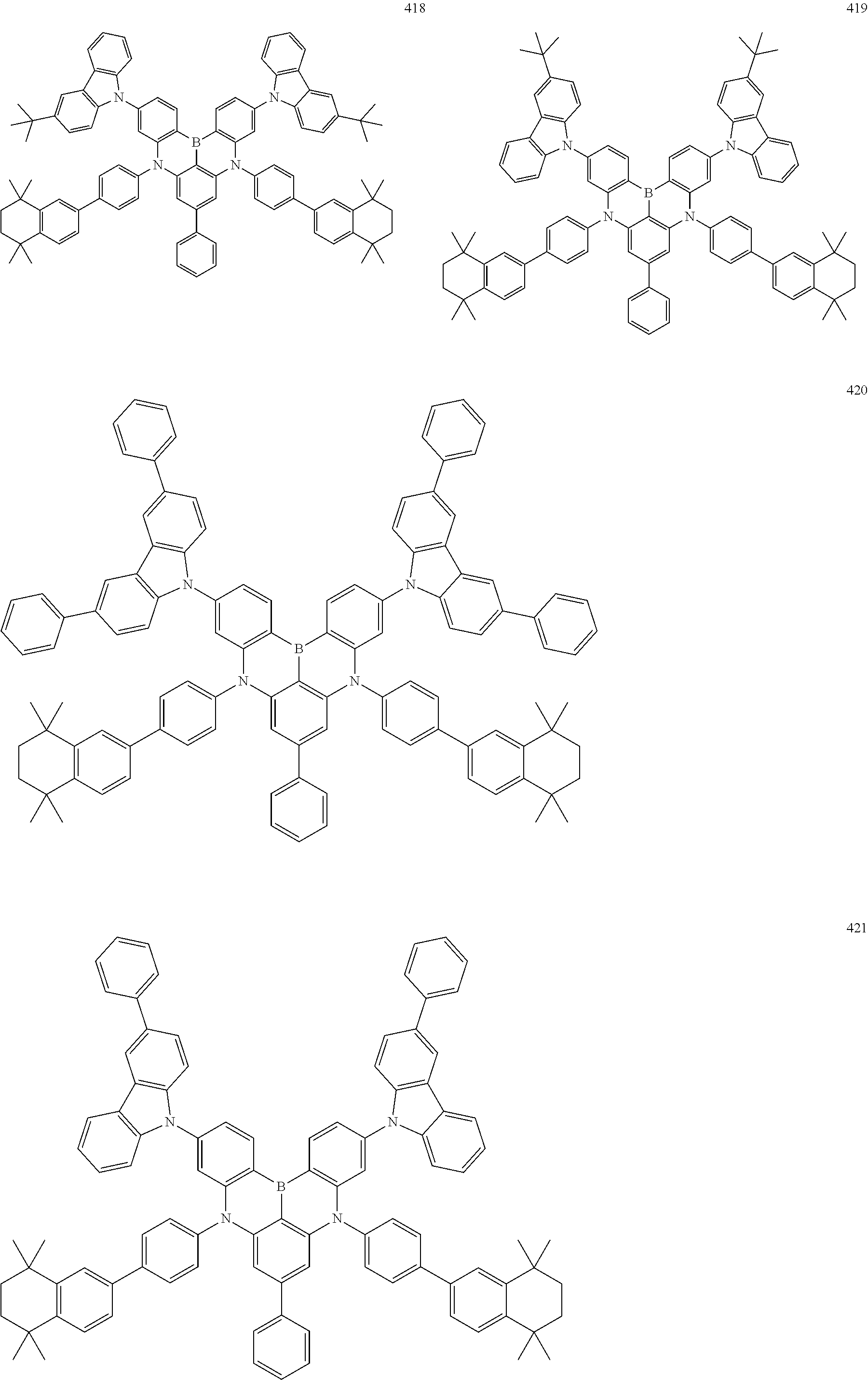

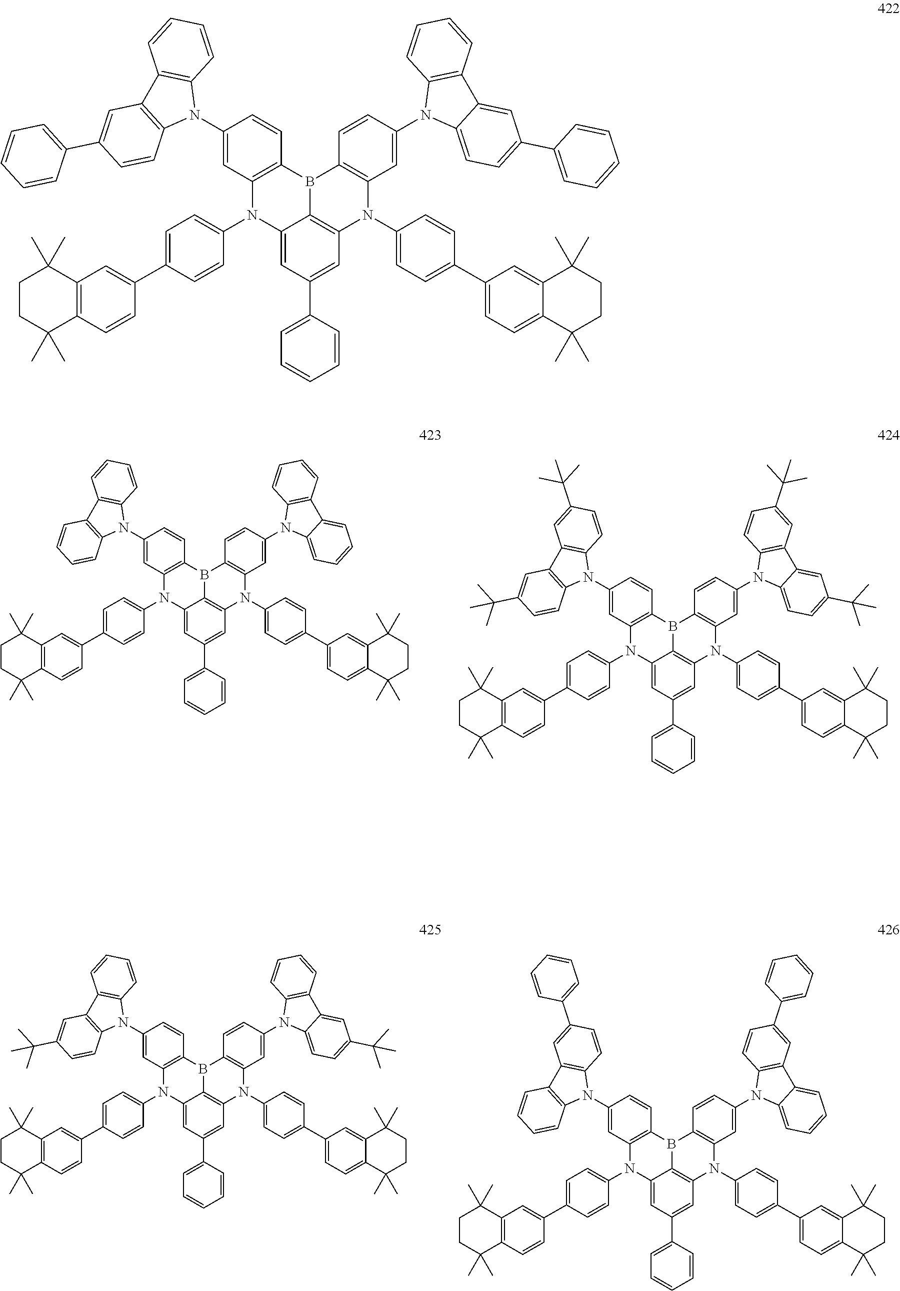

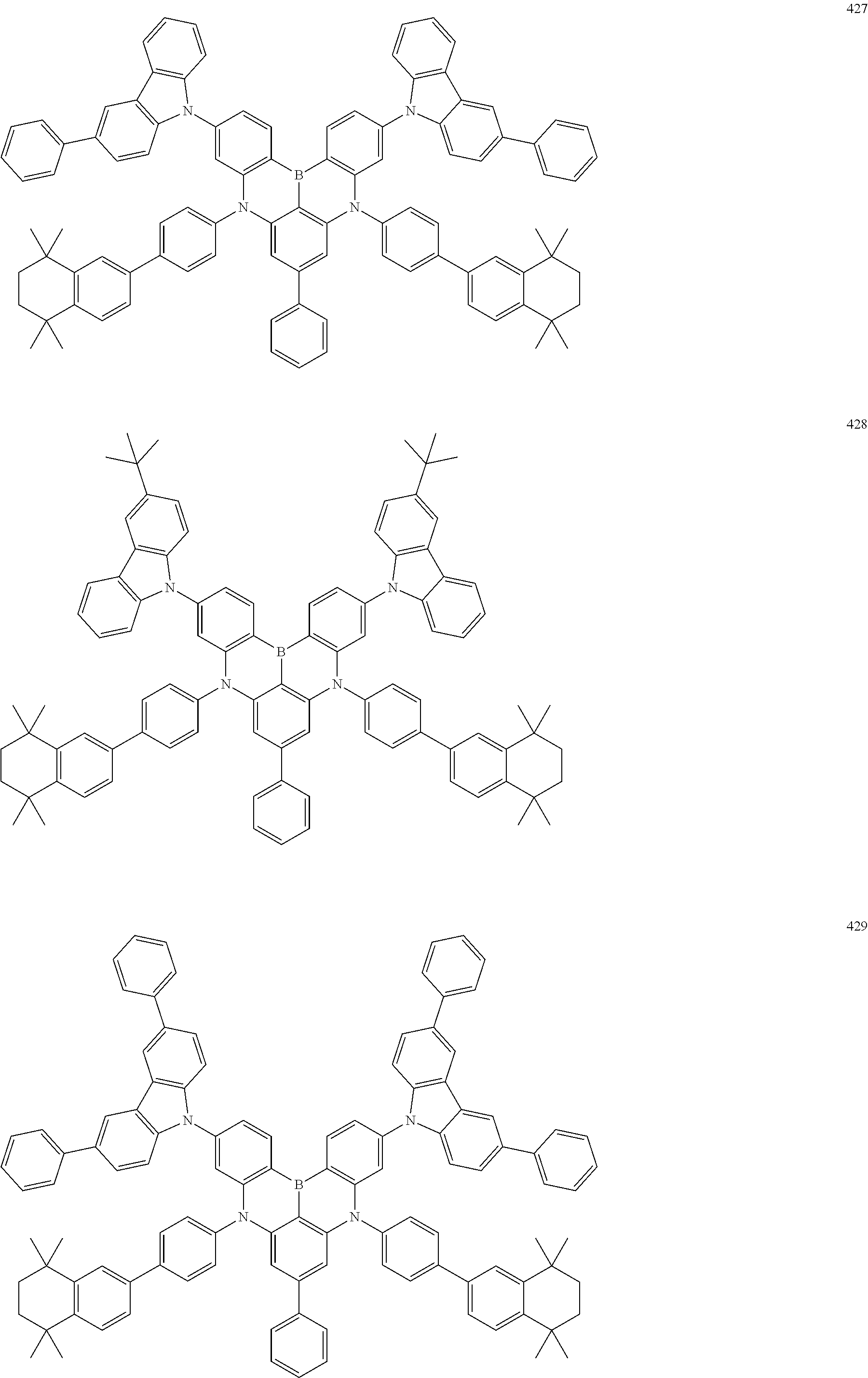

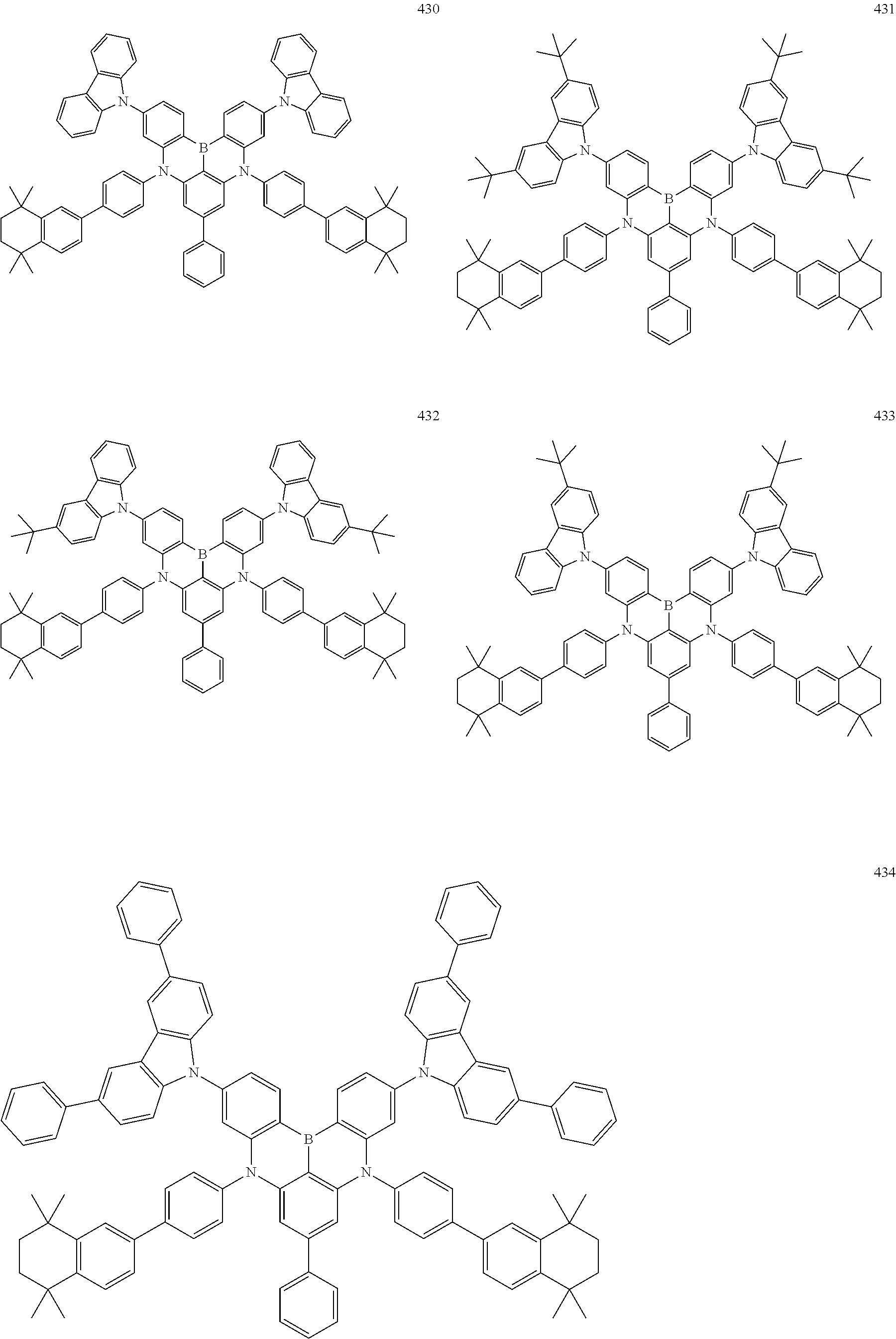

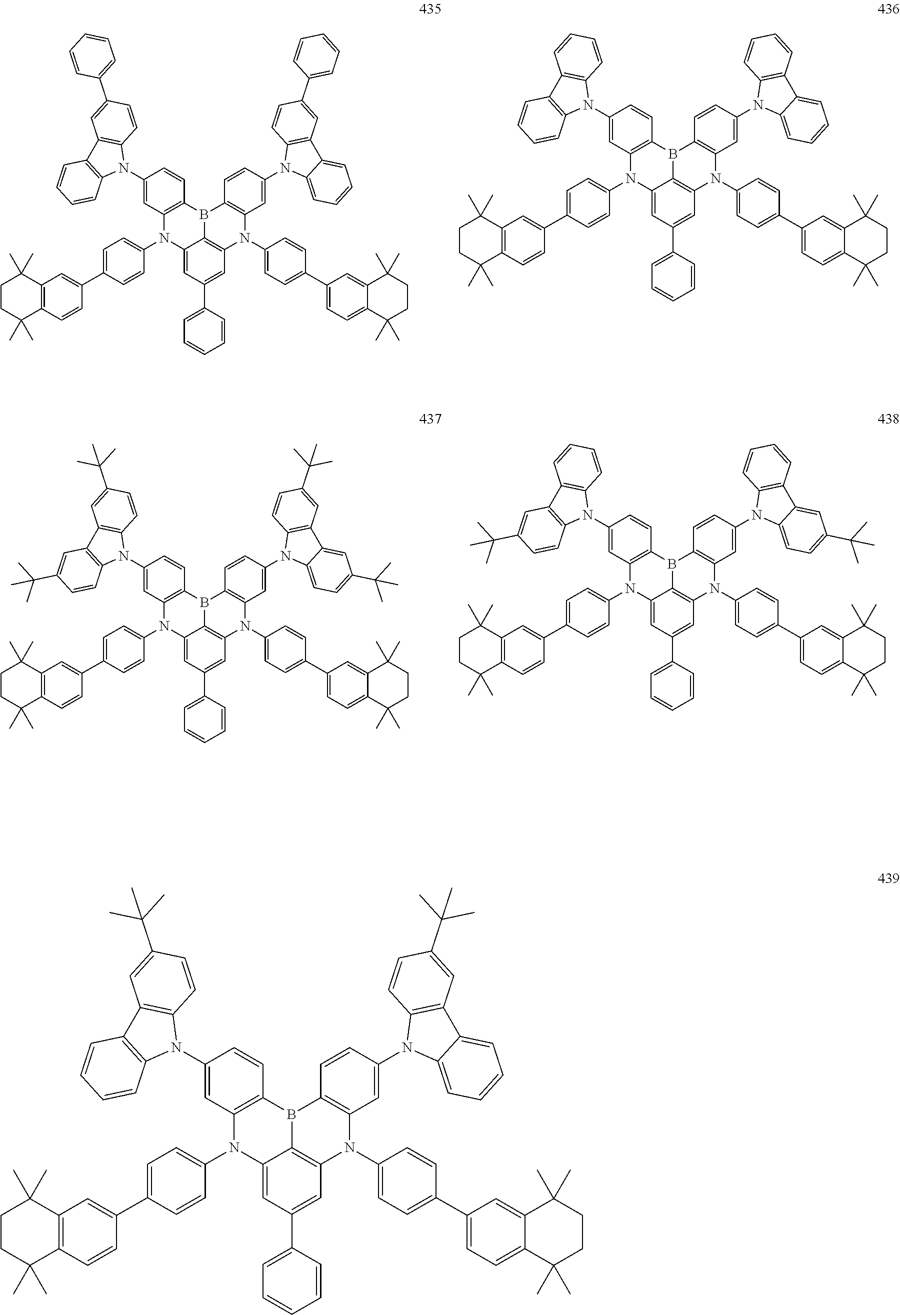

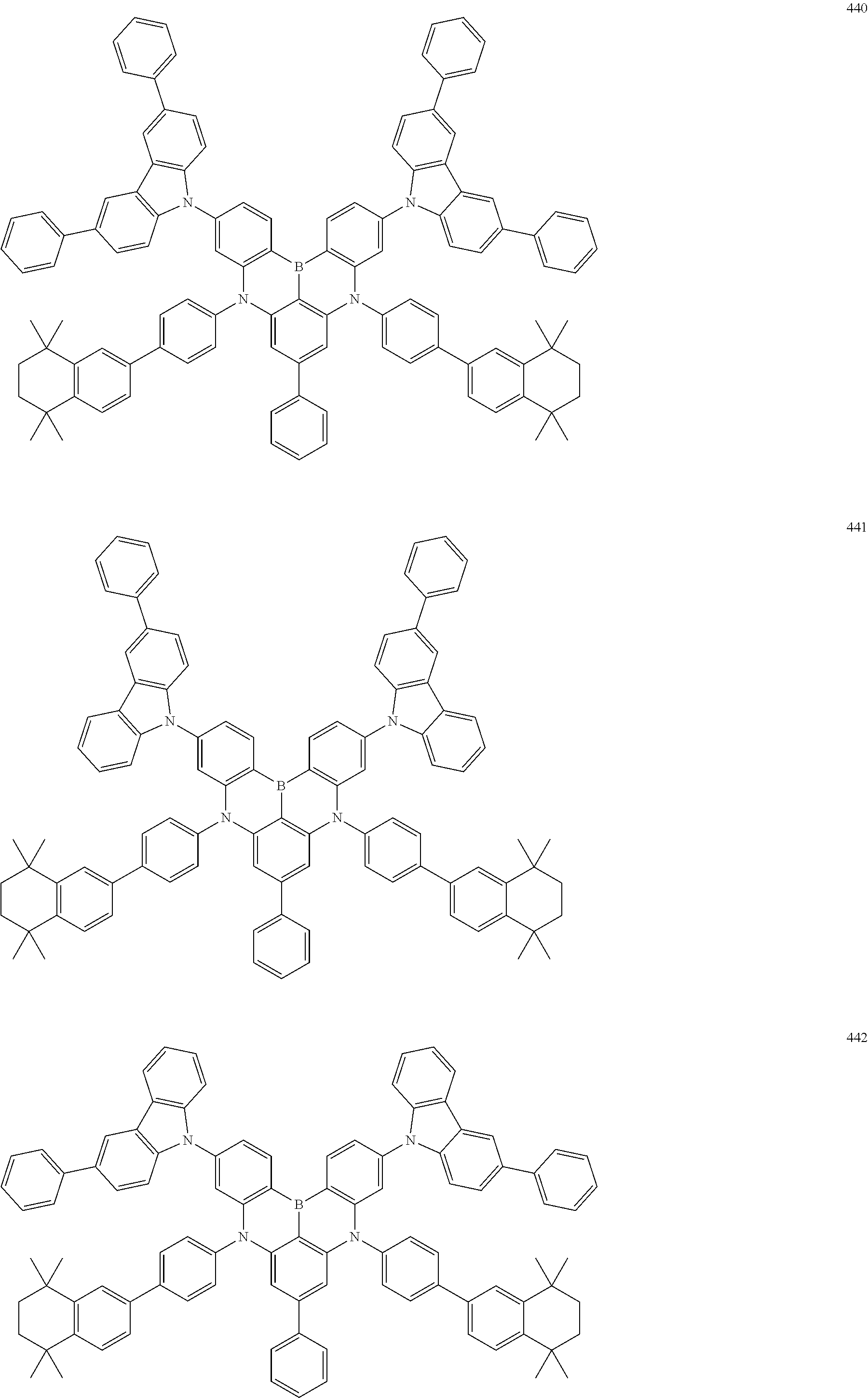

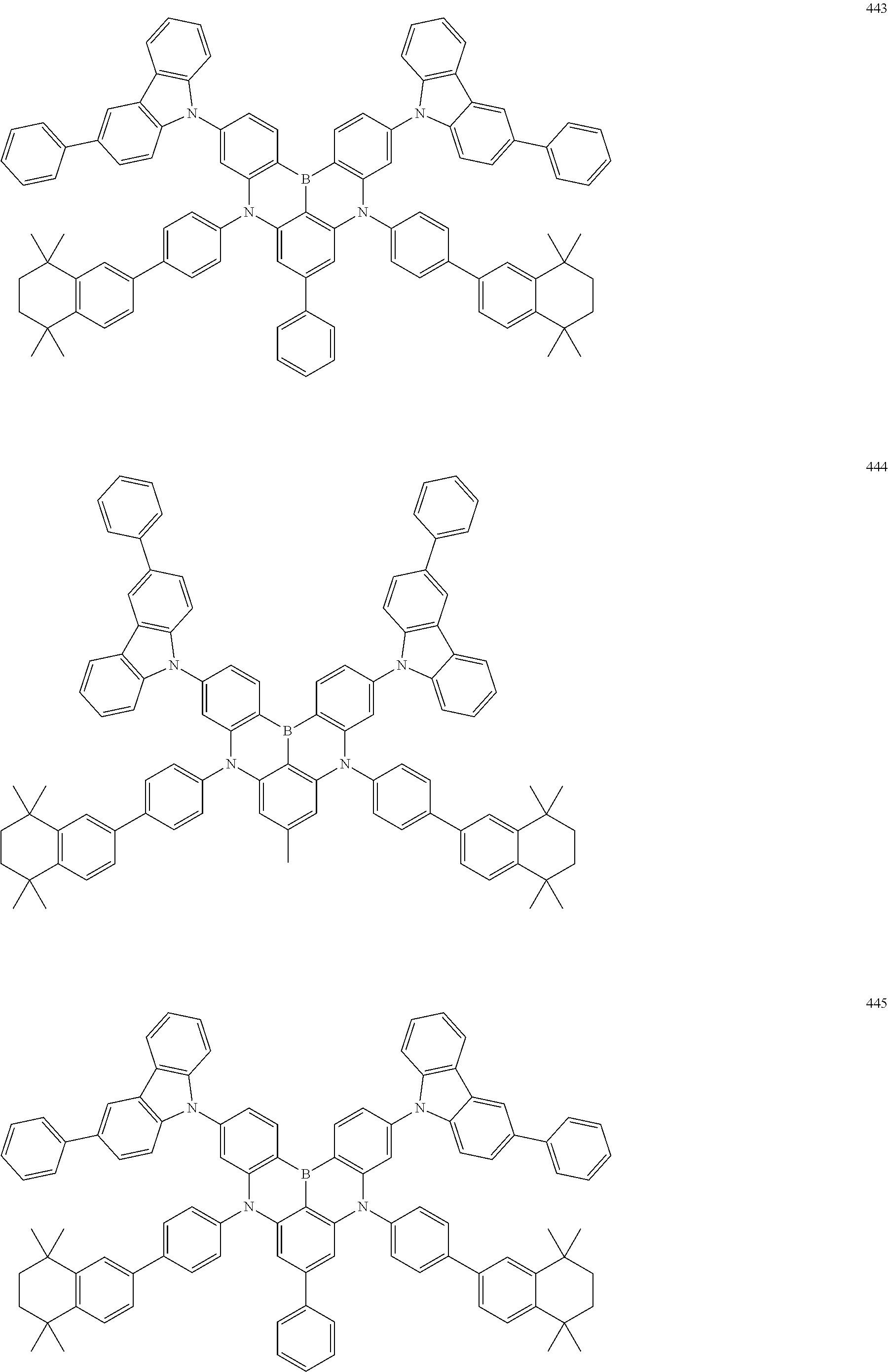

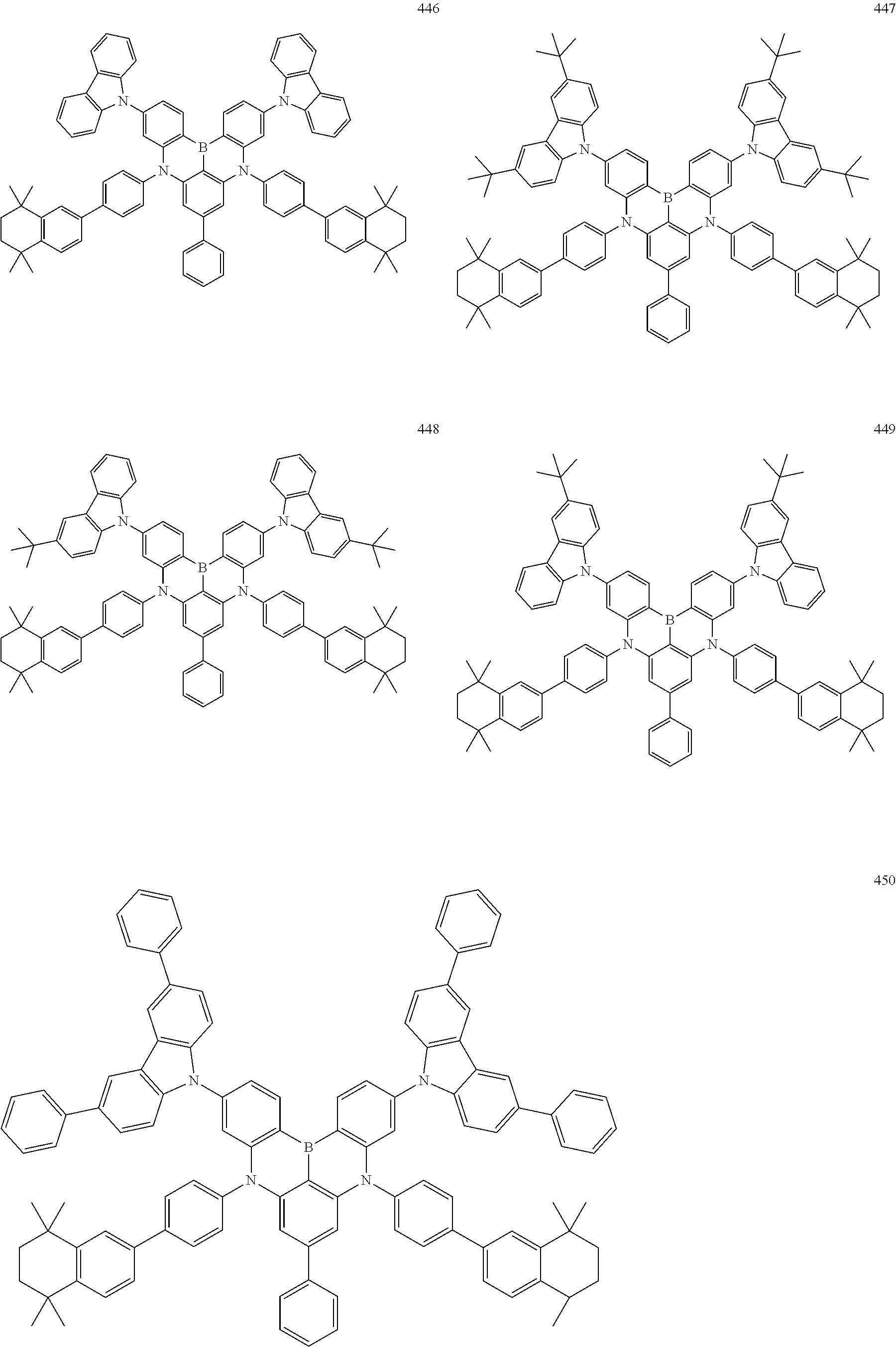

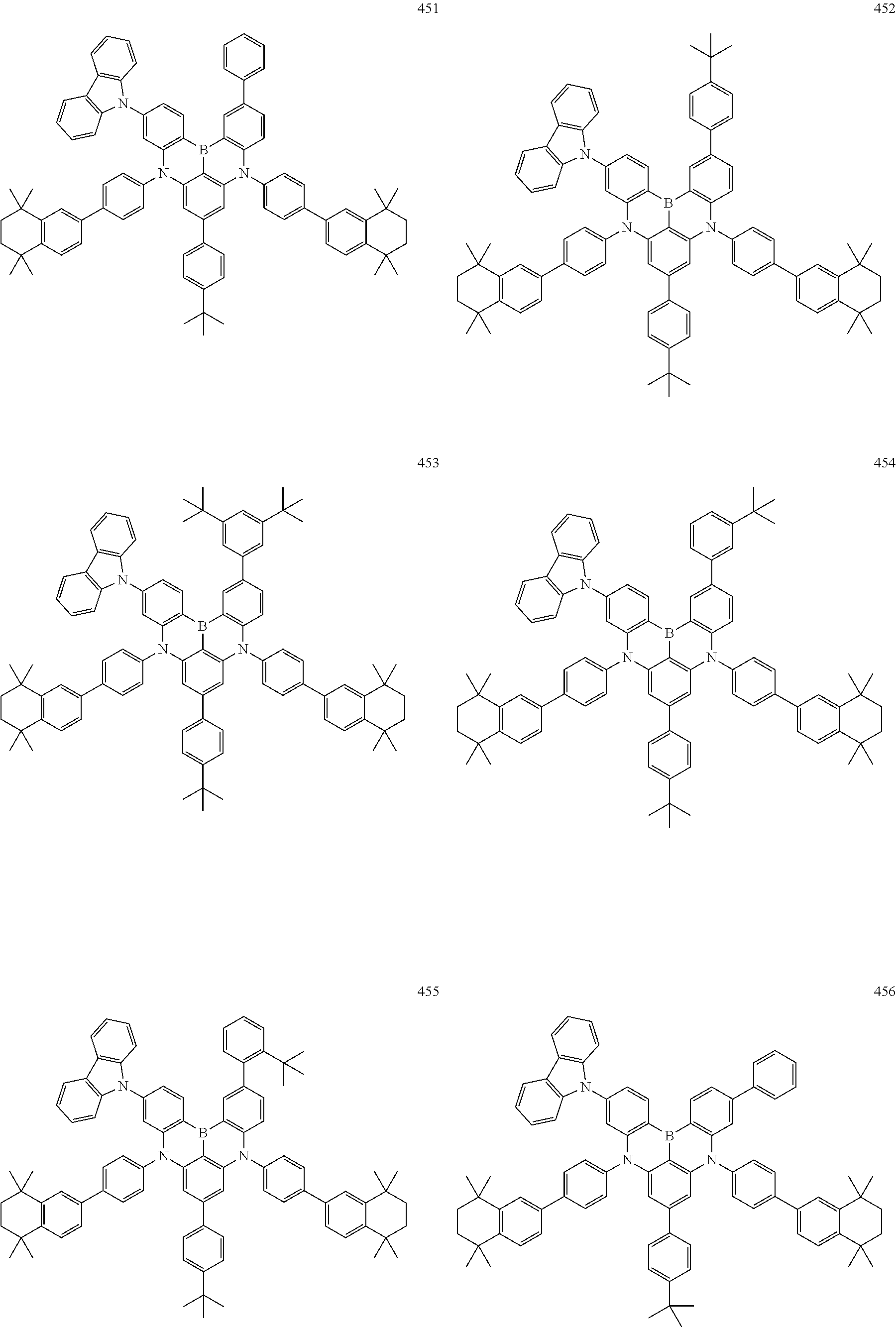

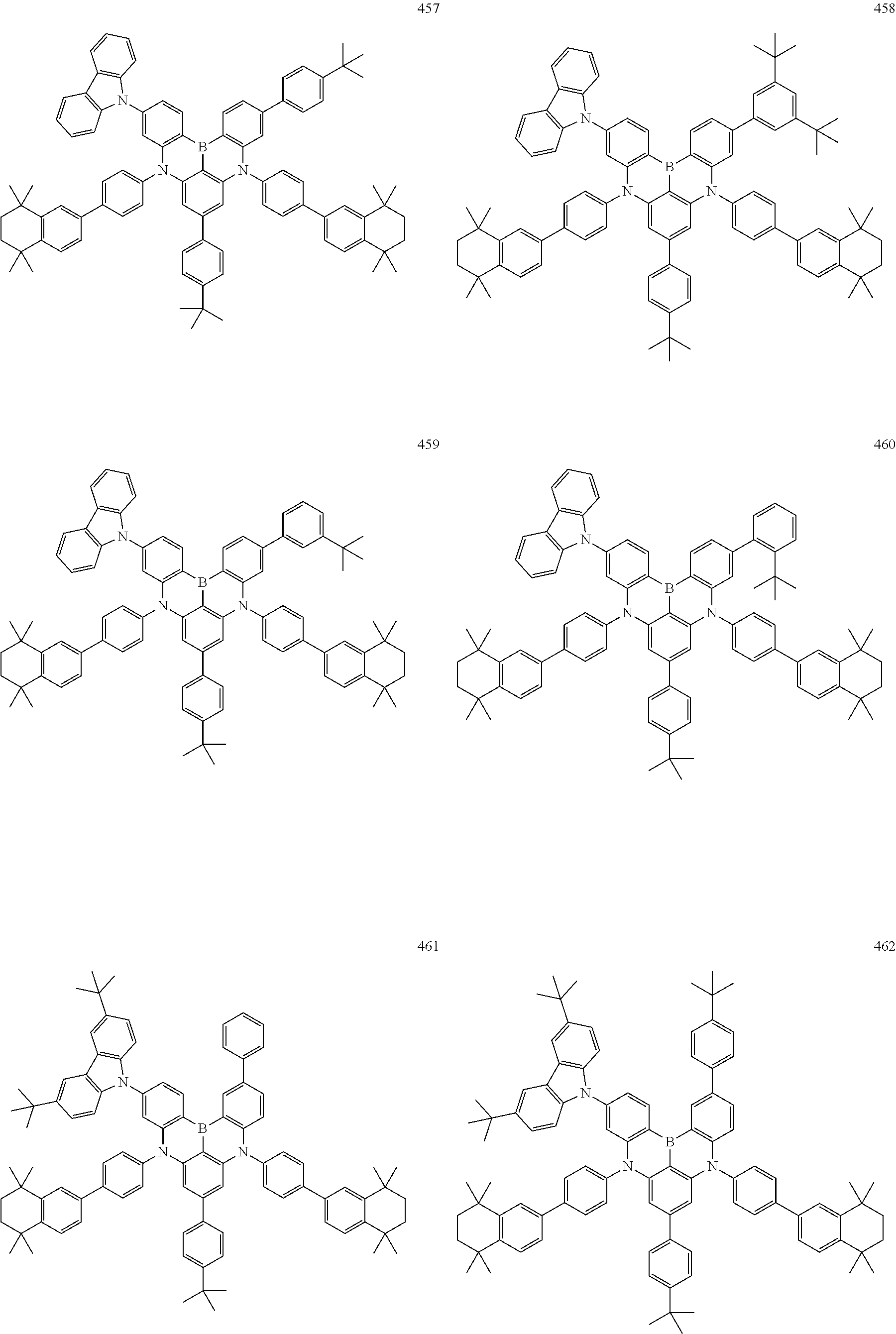

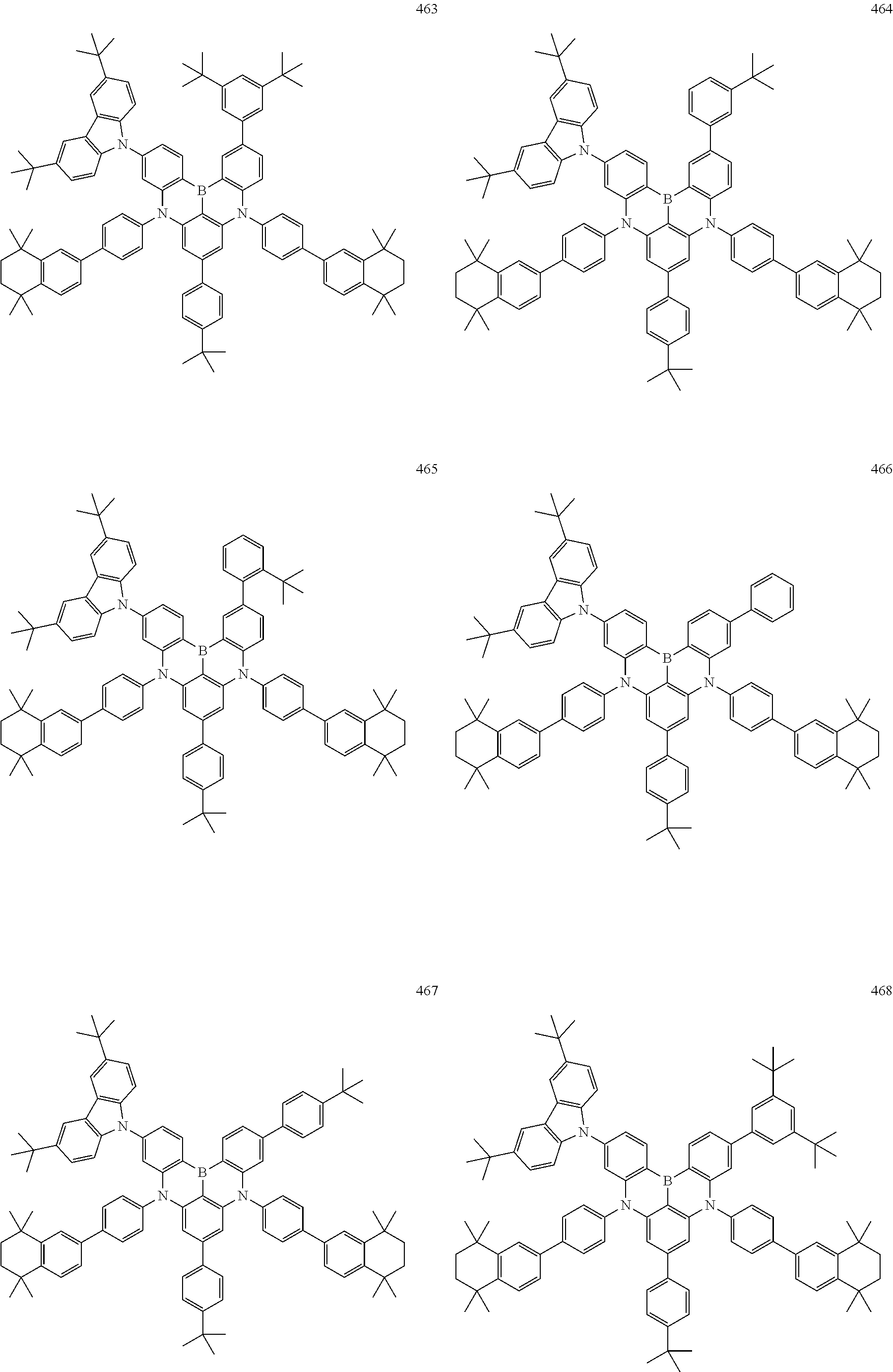

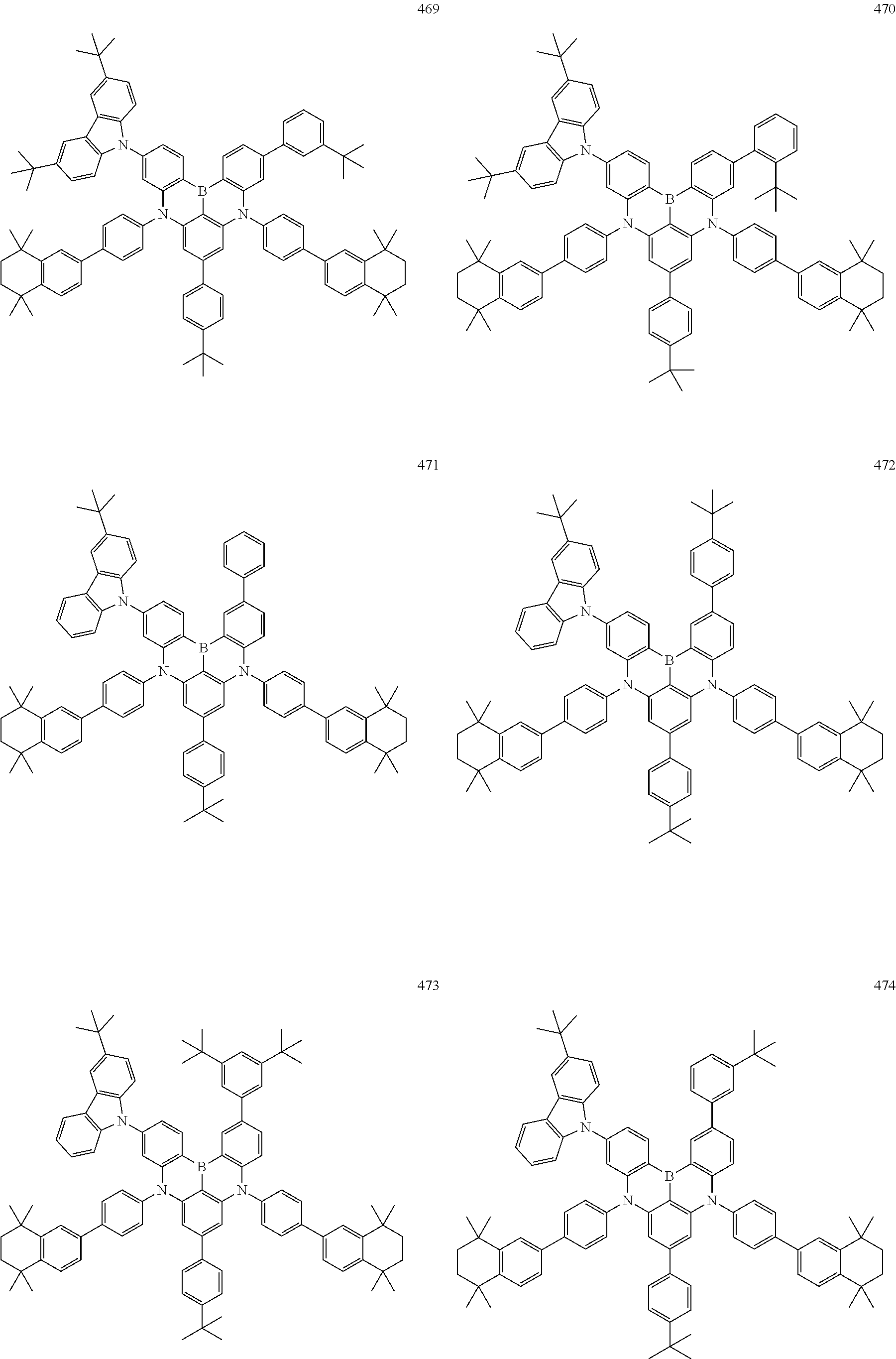

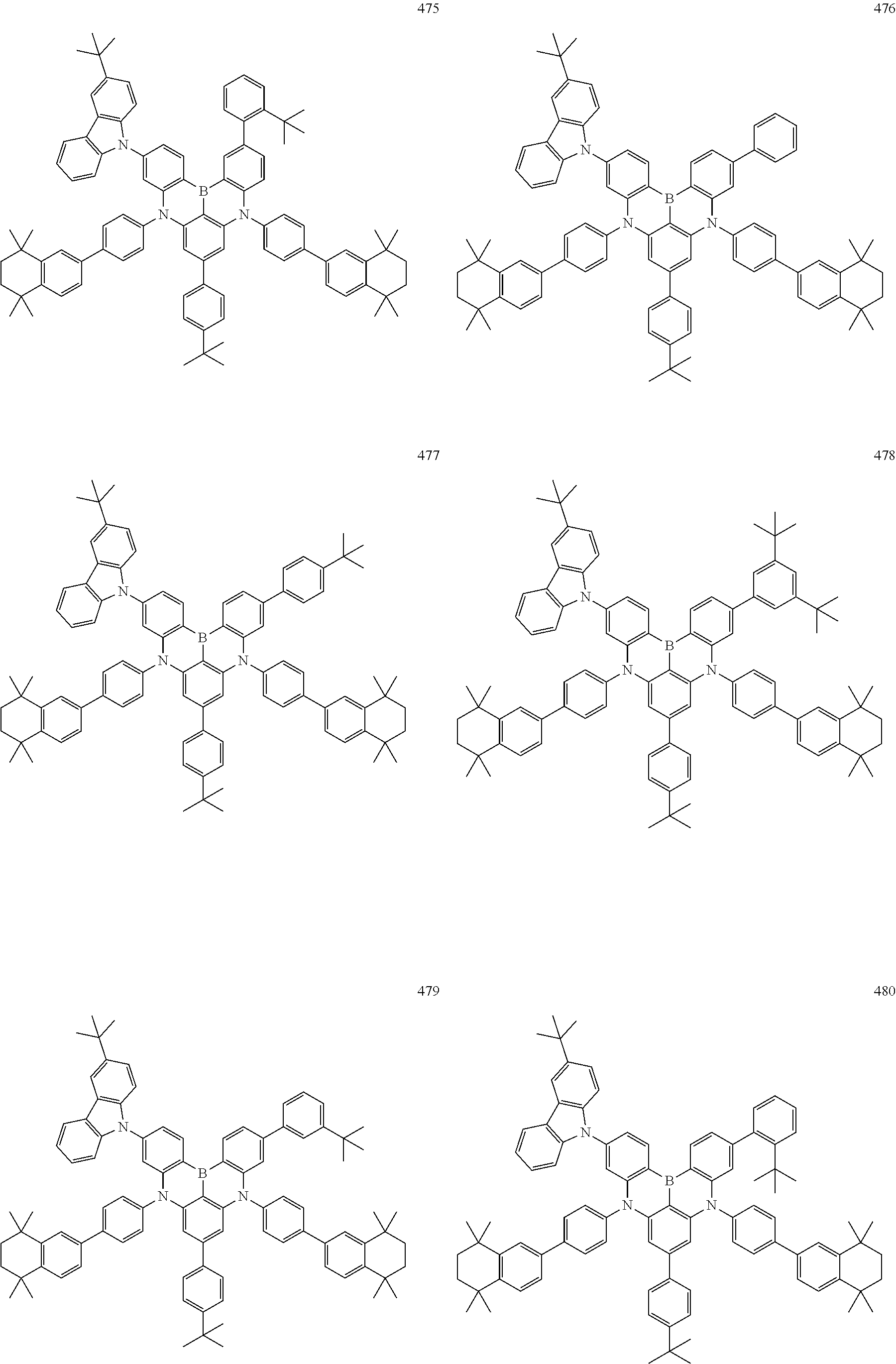

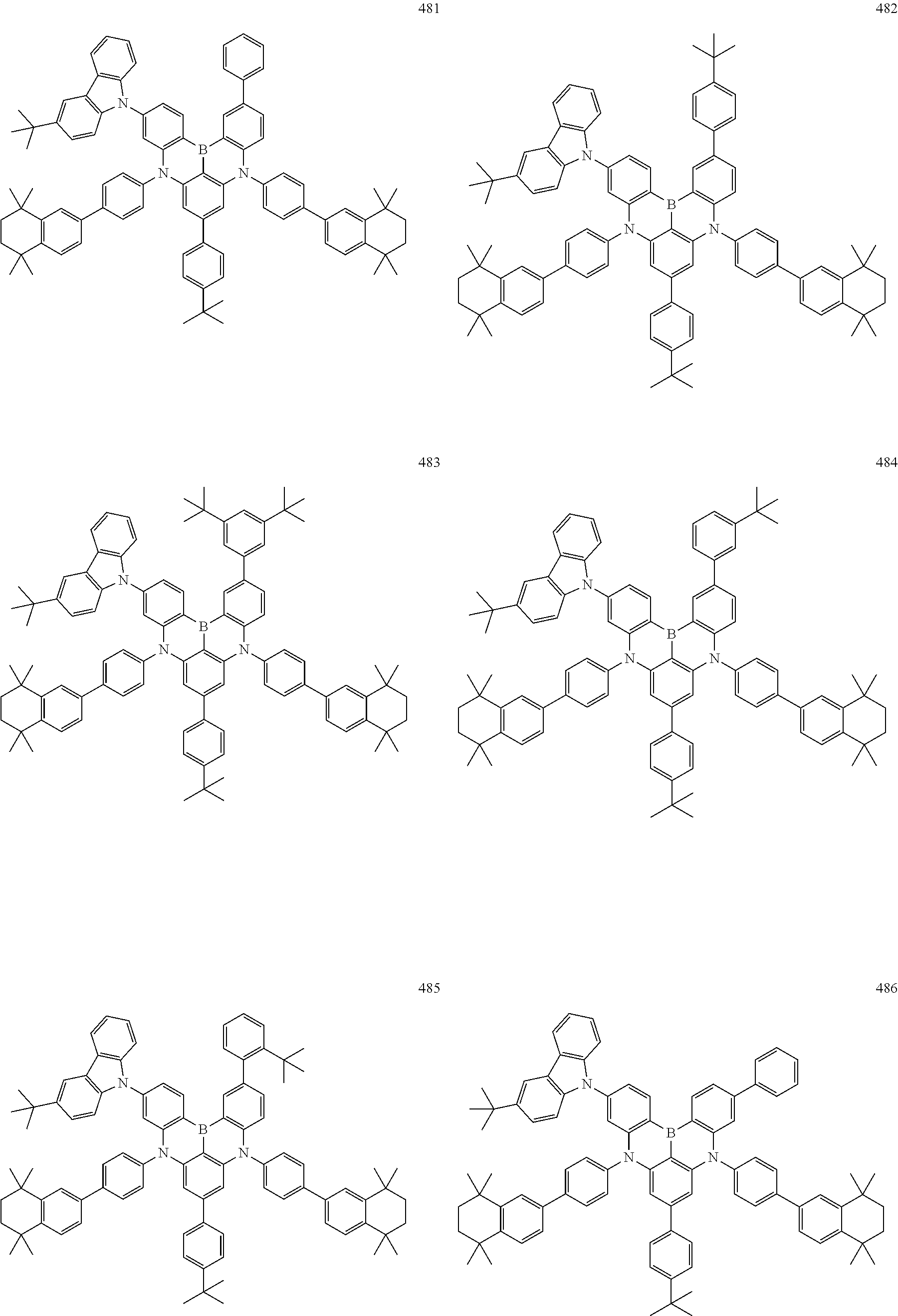

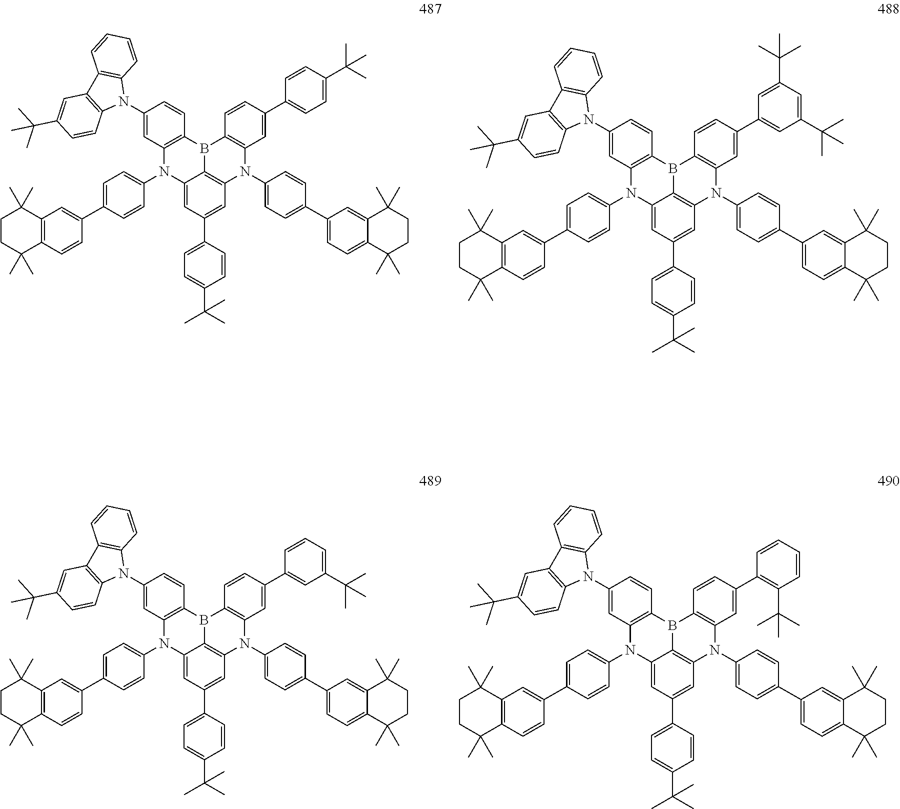

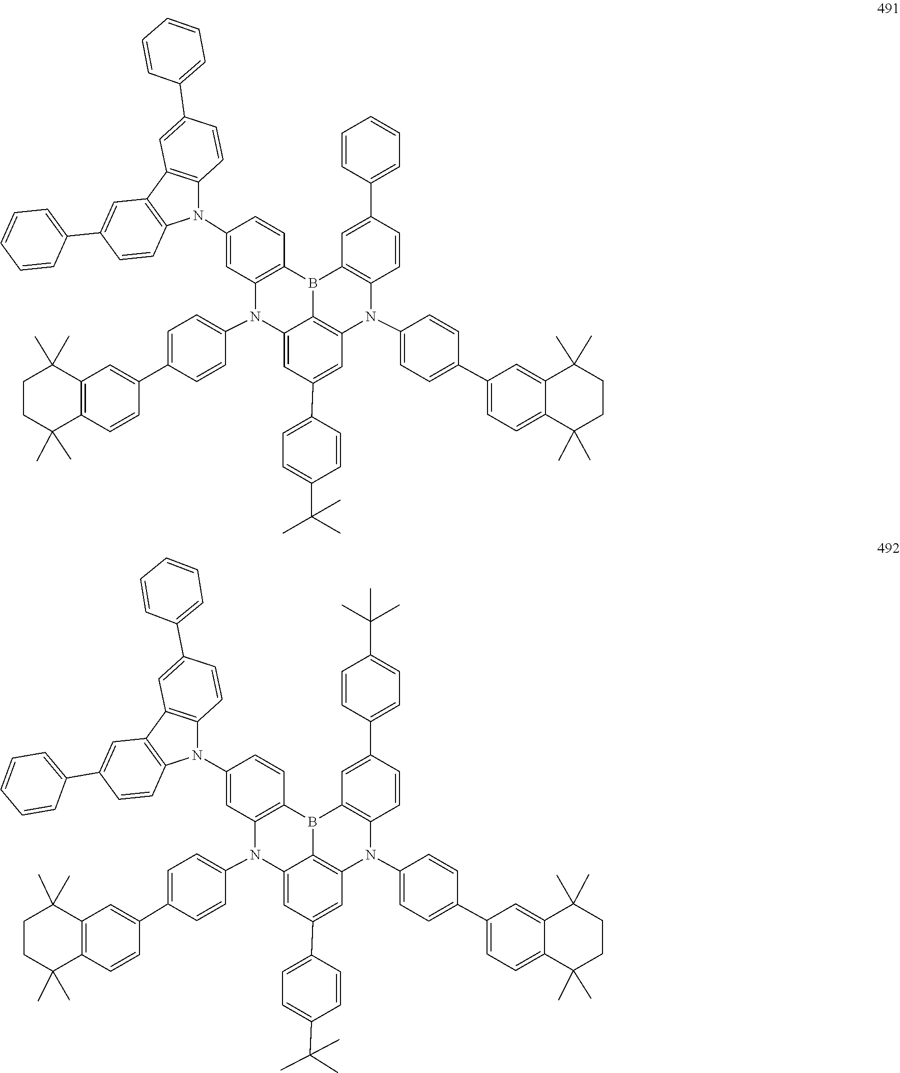 [SMILES: CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2cc(-c4ccccc4)ccc2N3c2ccc(-c3ccc4c(c3)C(C)(C)CCC4(C)C)cc2)cc1 ; CC(C)(C)c1ccc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CCC6(C)C)cc4)c4cc(-c5ccc(C(C)(C)C)cc5)cc(c42)N3c2ccc(-c3ccc4c(c3)C(C)(C)CCC4(C)C)cc2)cc1]

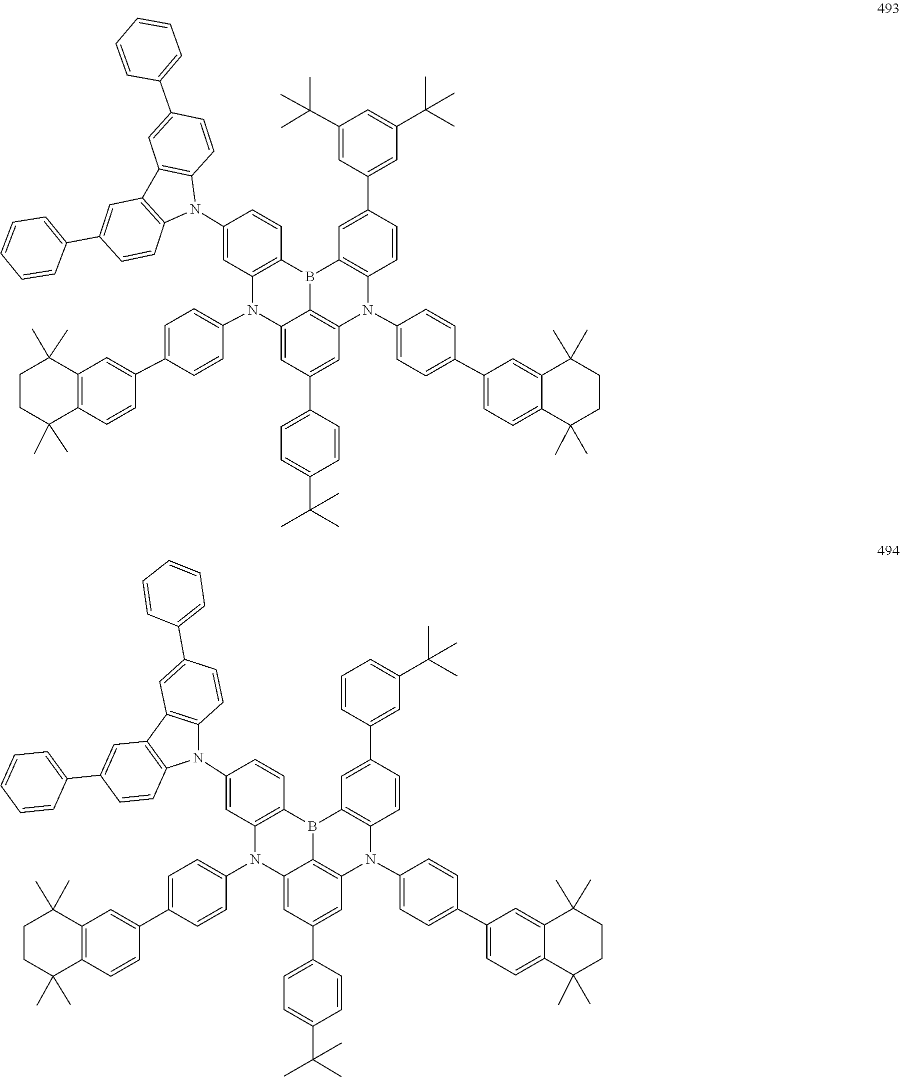 [SMILES: CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2cc(-c4cc(C(C)(C)C)cc(C(C)(C)C)c4)ccc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2cc(-c4cccc(C(C)(C)C)c4)ccc2N3c2ccc(-c3ccc4c(c3)C(C)(C)CCC4(C)C)cc2)cc1]

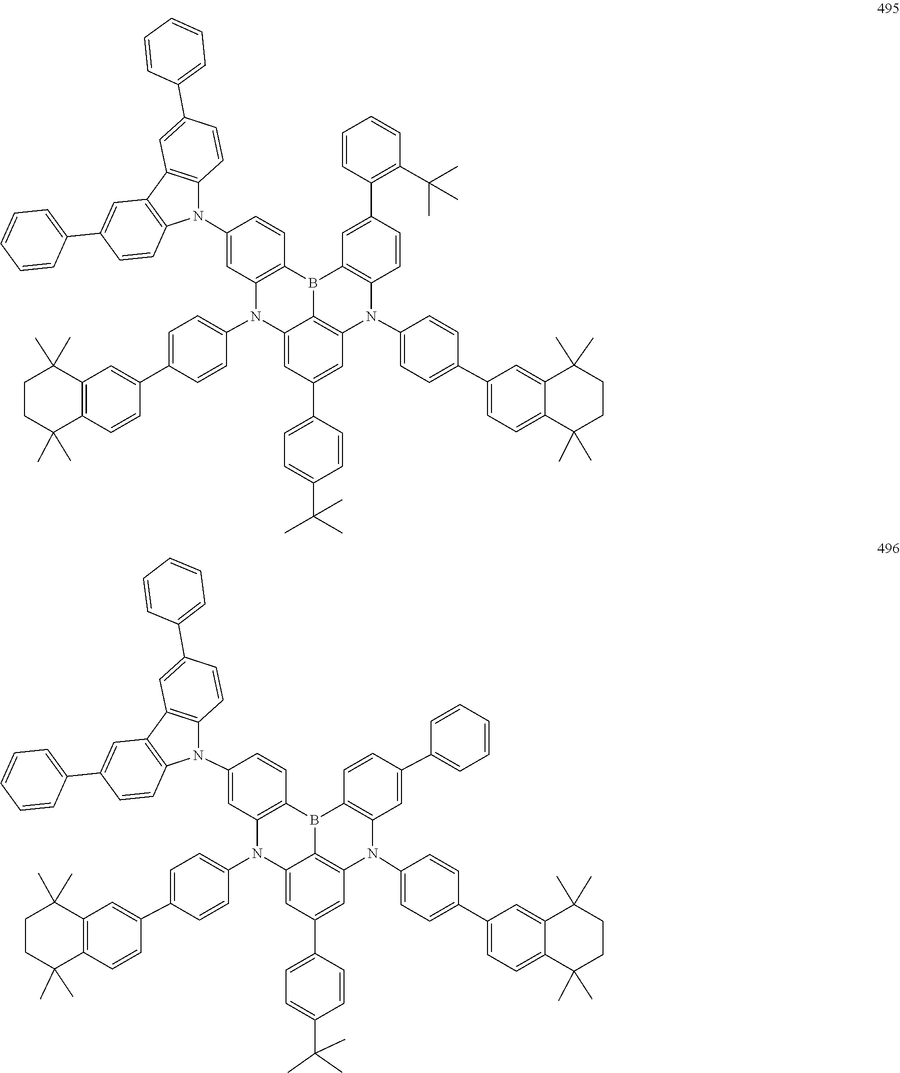 [SMILES: CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2cc(-c4ccccc4C(C)(C)C)ccc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2ccc(-c4ccccc4)cc2N3c2ccc(-c3ccc4c(c3)C(C)(C)CCC4(C)C)cc2)cc1]

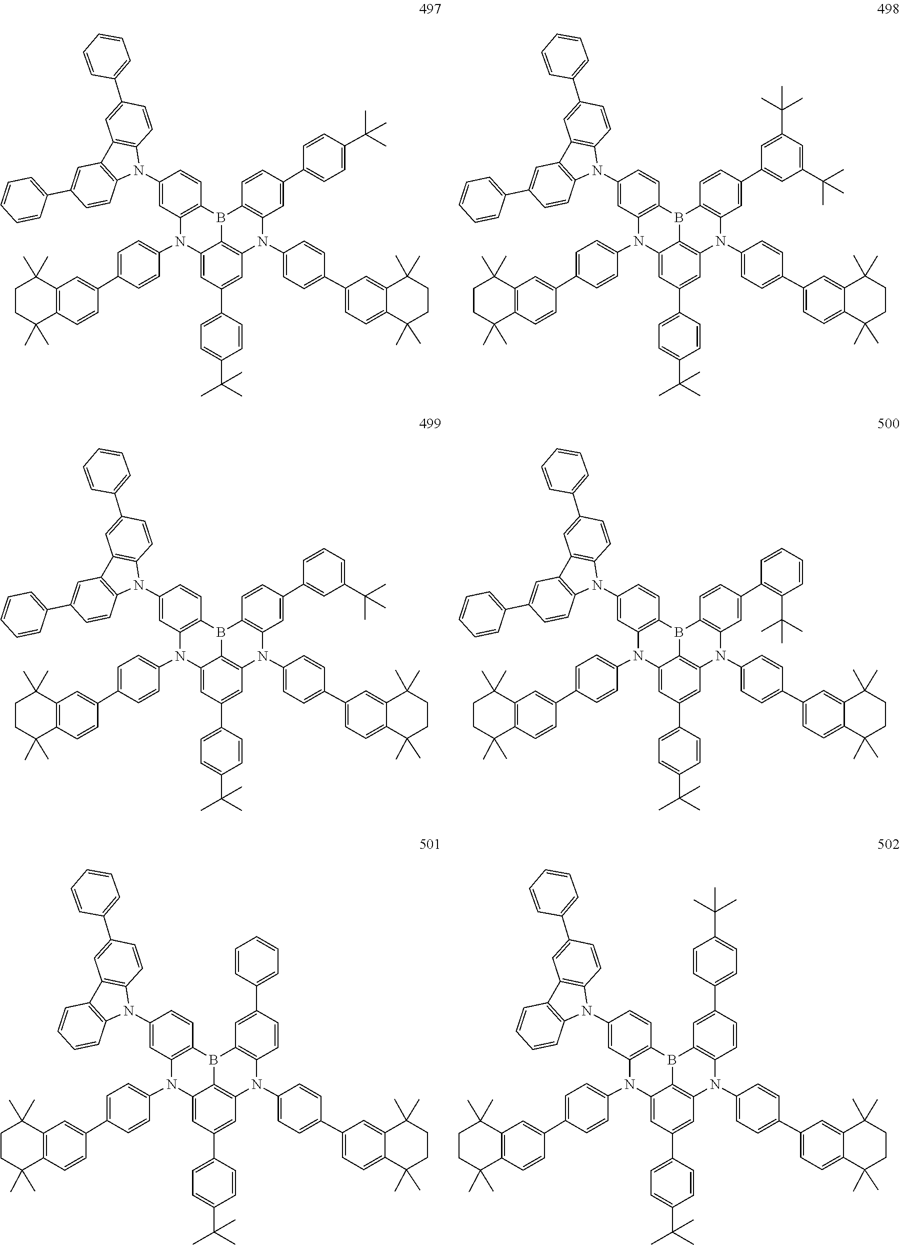

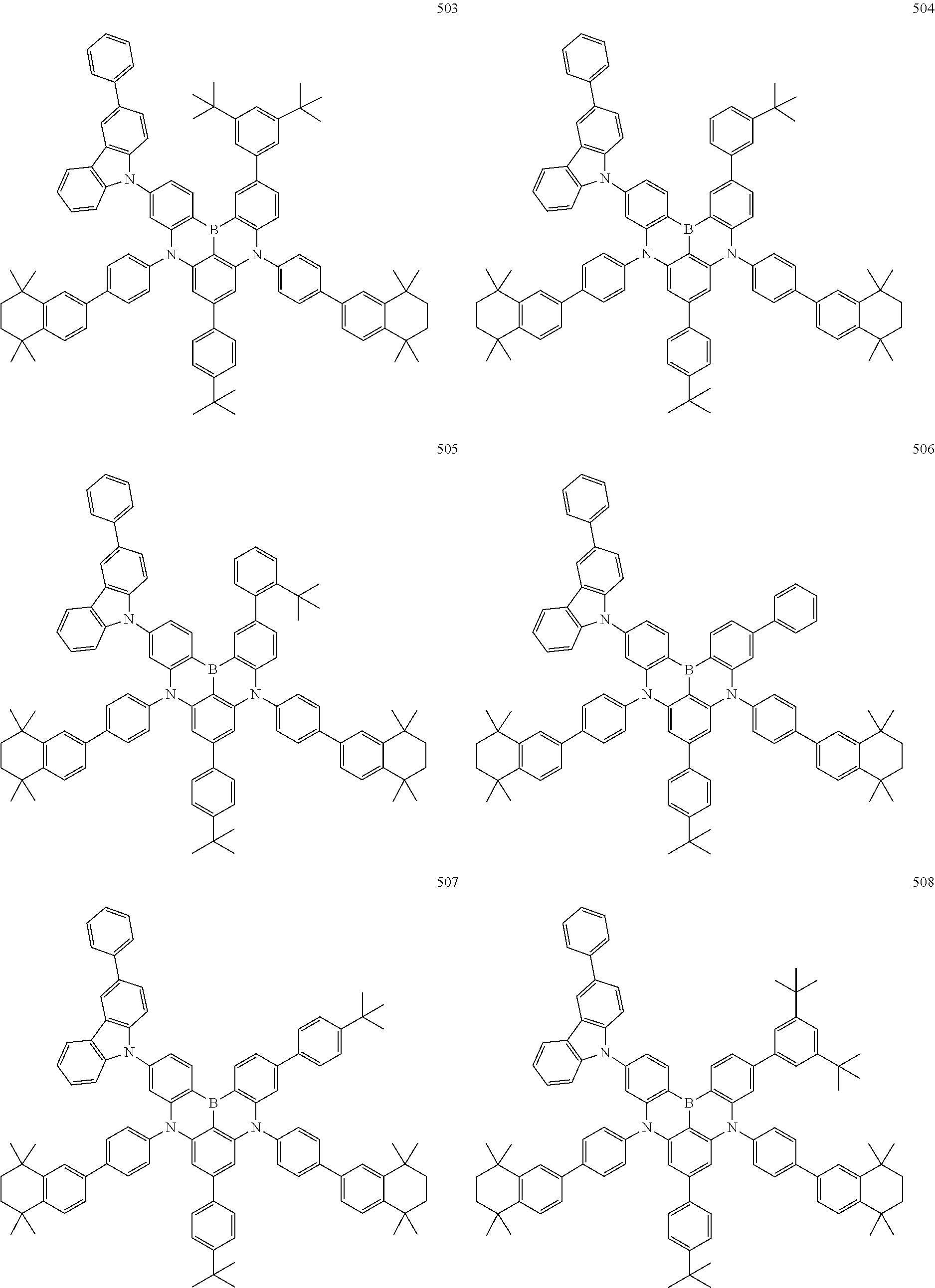

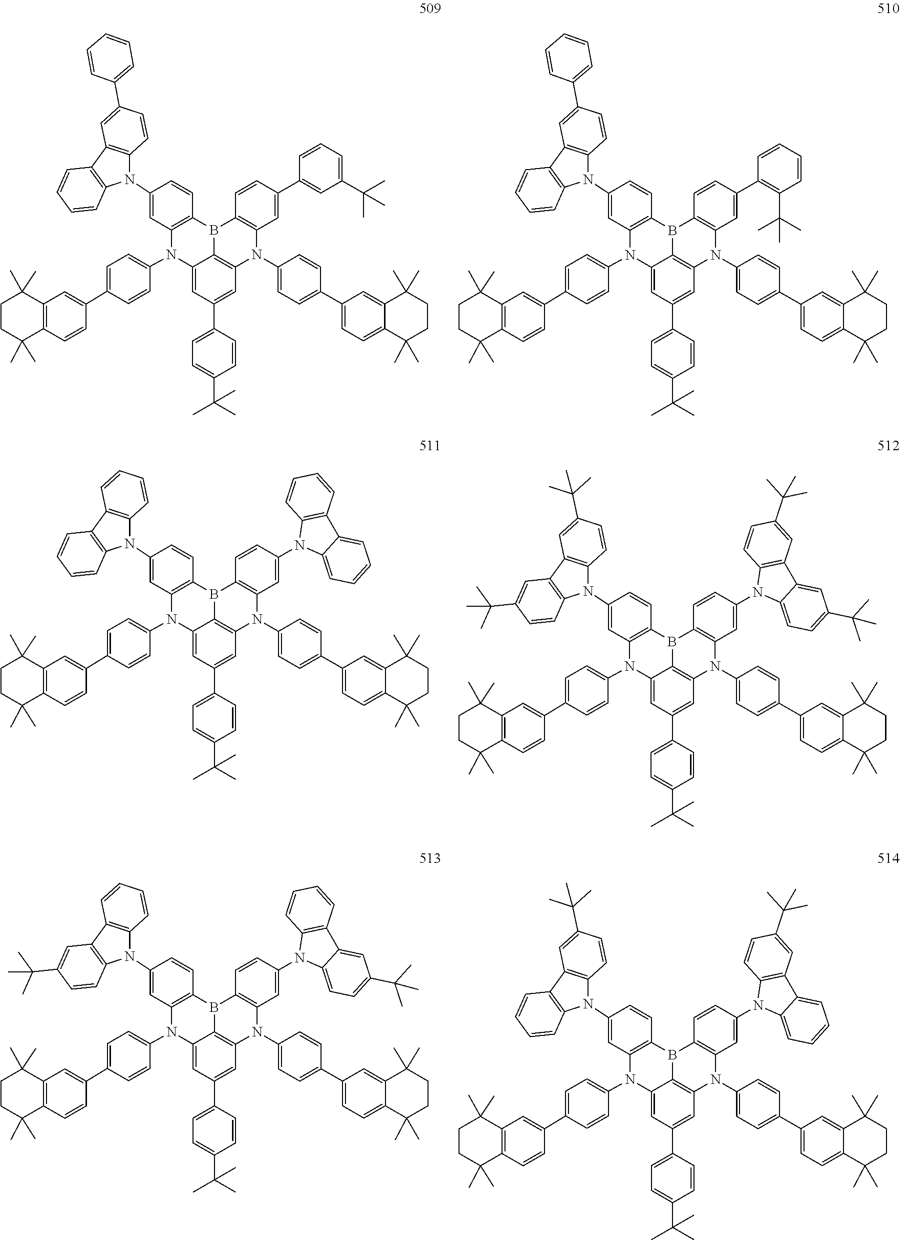

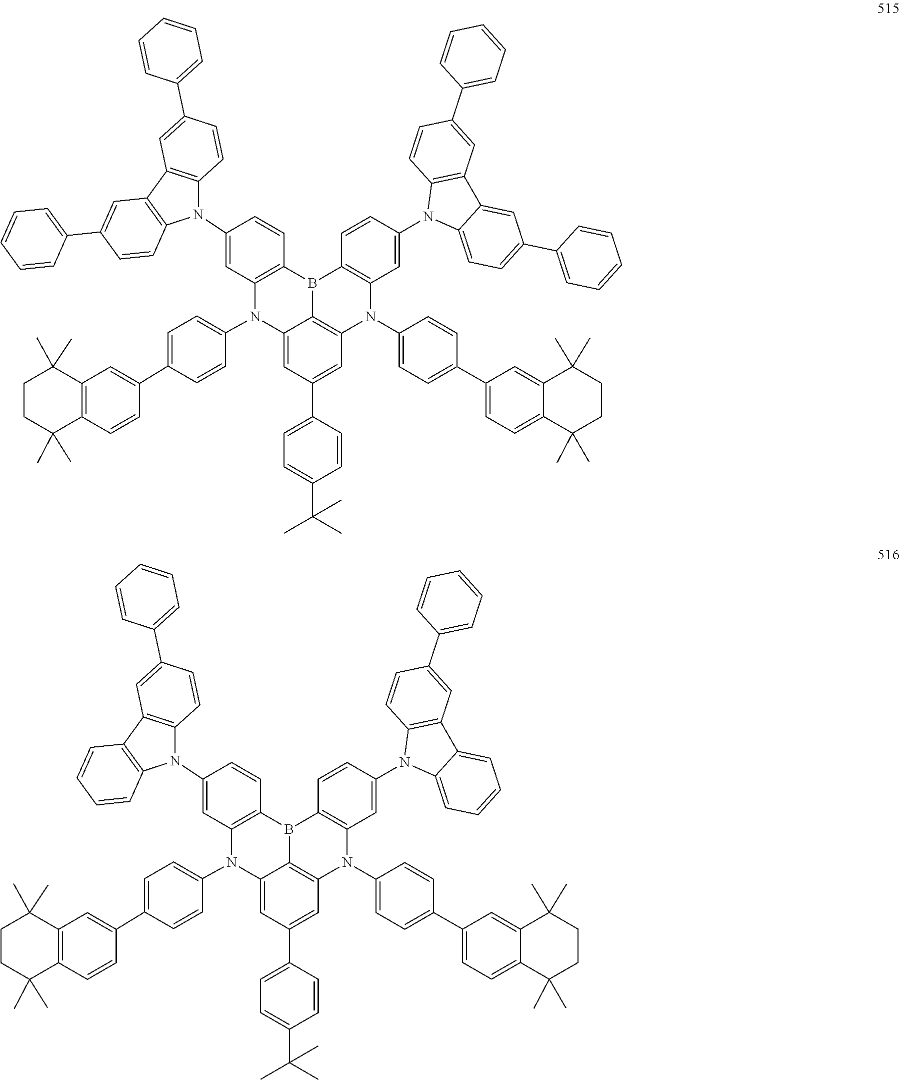 [SMILES: CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2ccc(-n4c5ccc(-c6ccccc6)cc5c5cc(-c6ccccc6)ccc54)cc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccccc6c6cc(-c7ccccc7)ccc65)ccc2B4c2ccc(-n4c5ccccc5c5cc(-c6ccccc6)ccc54)cc2N3c2ccc(-c3ccc4c(c3)C(C)(C)CCC4(C)C)cc2)cc1]

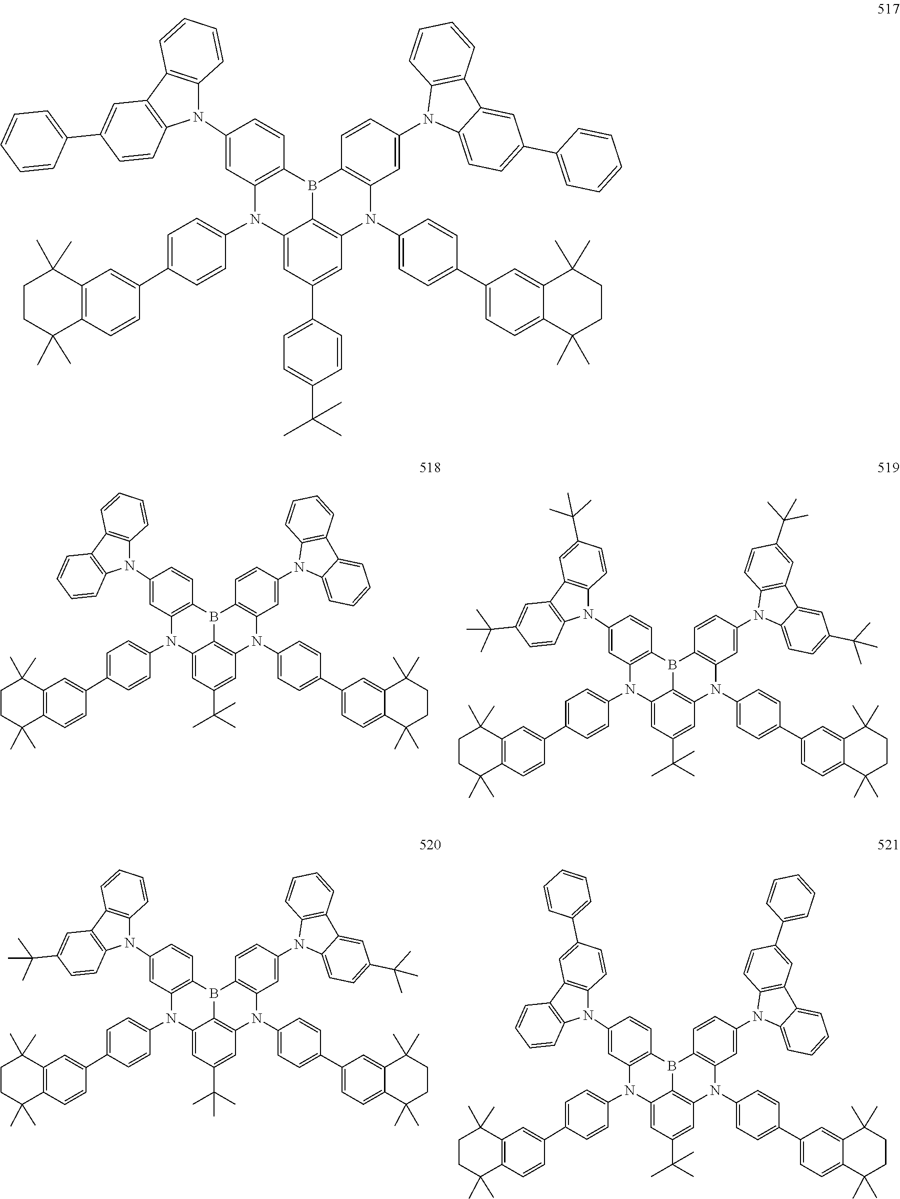

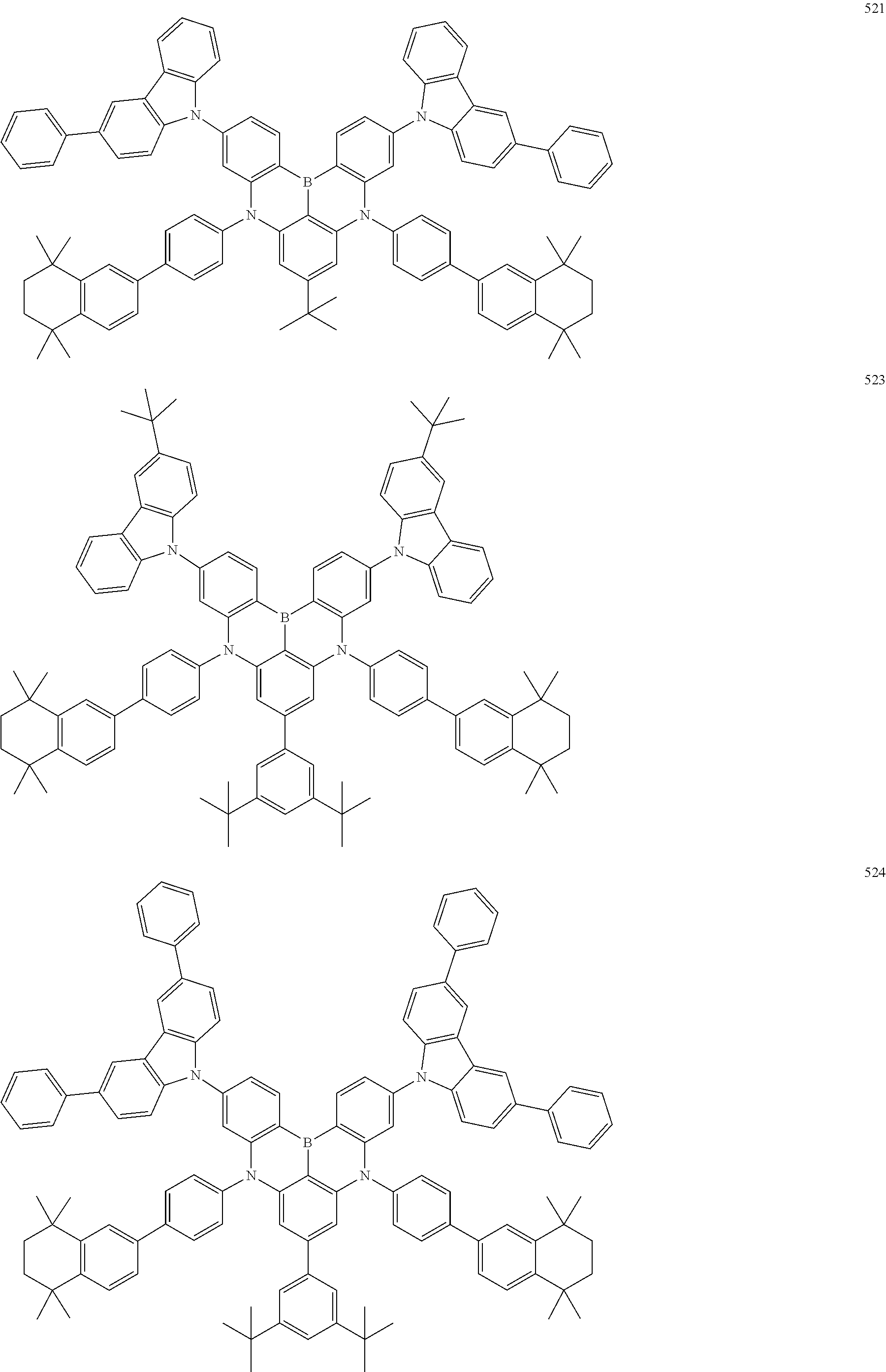

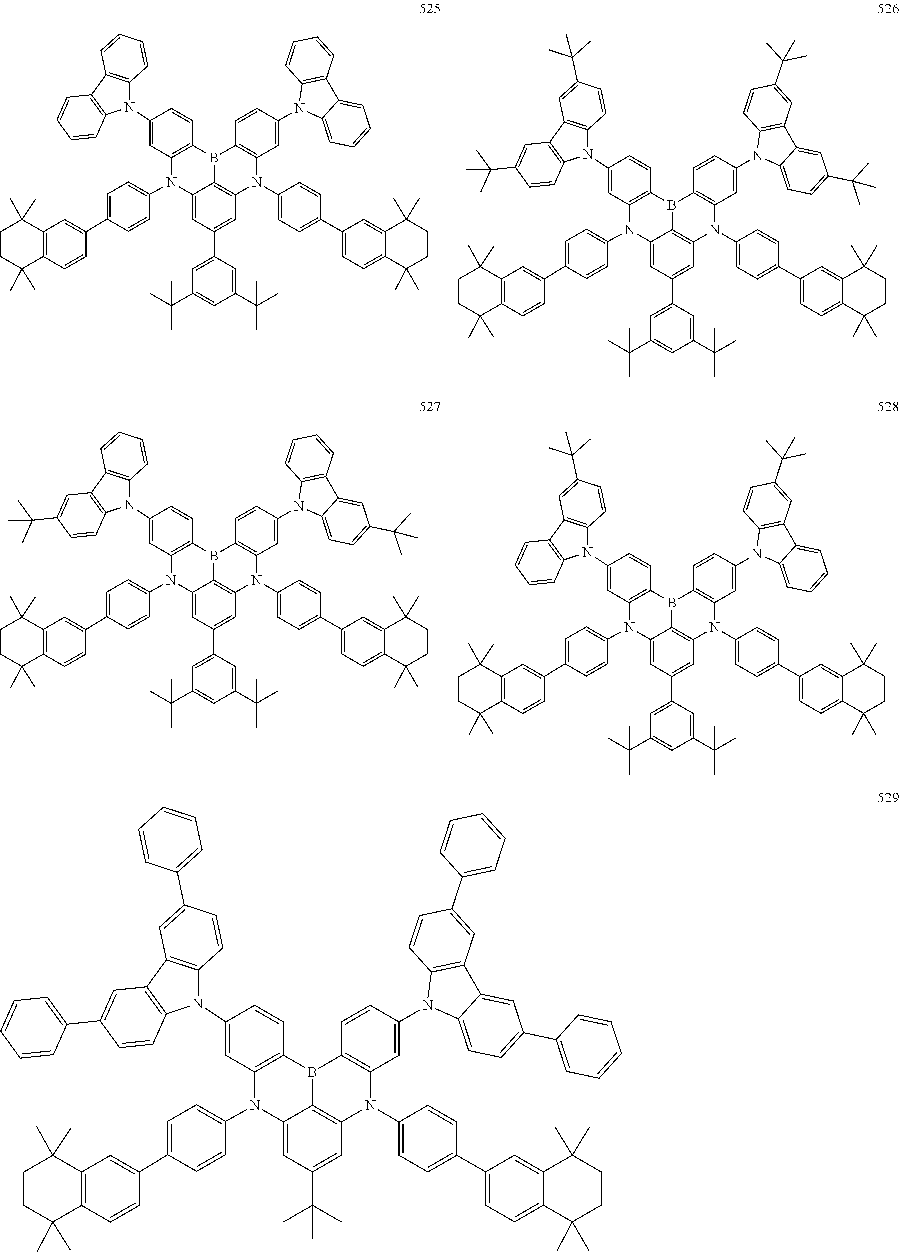

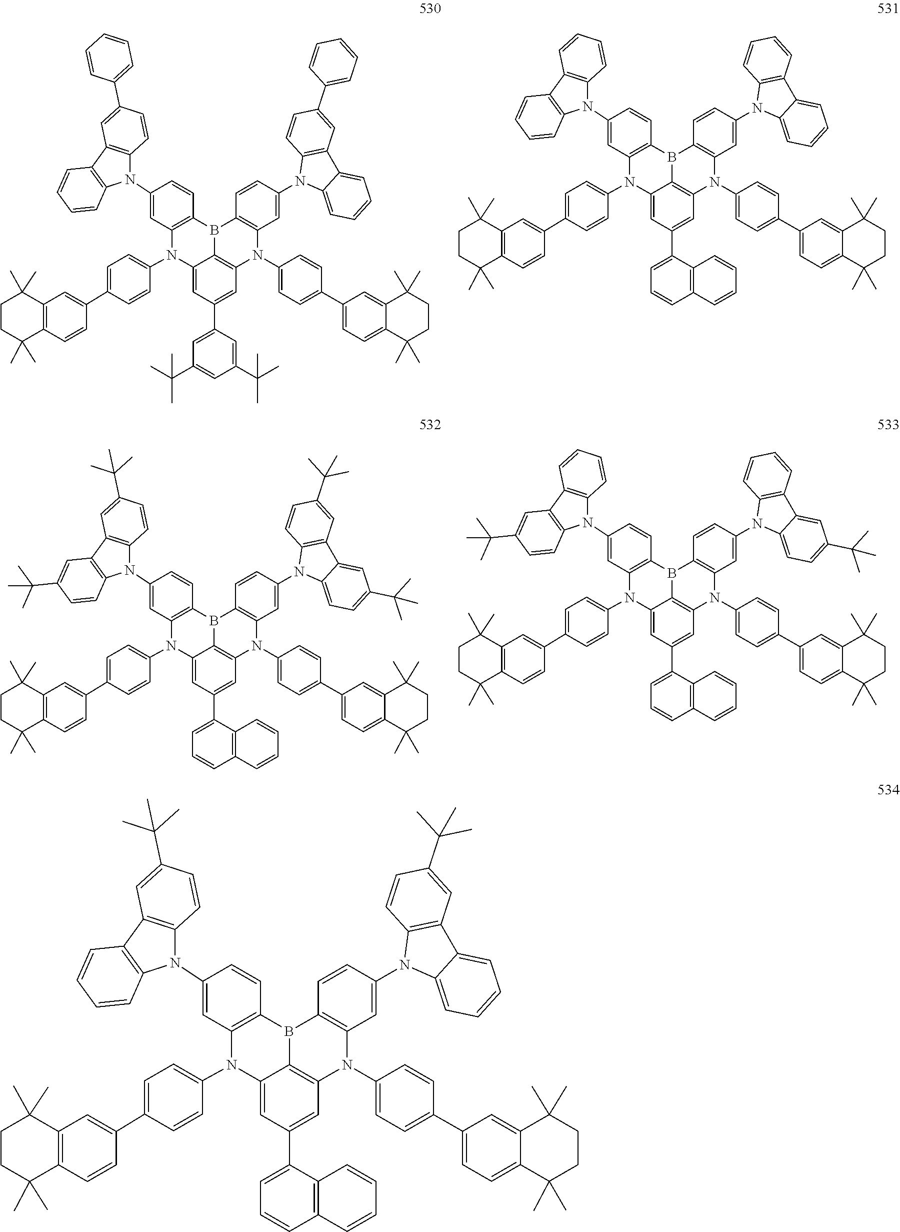

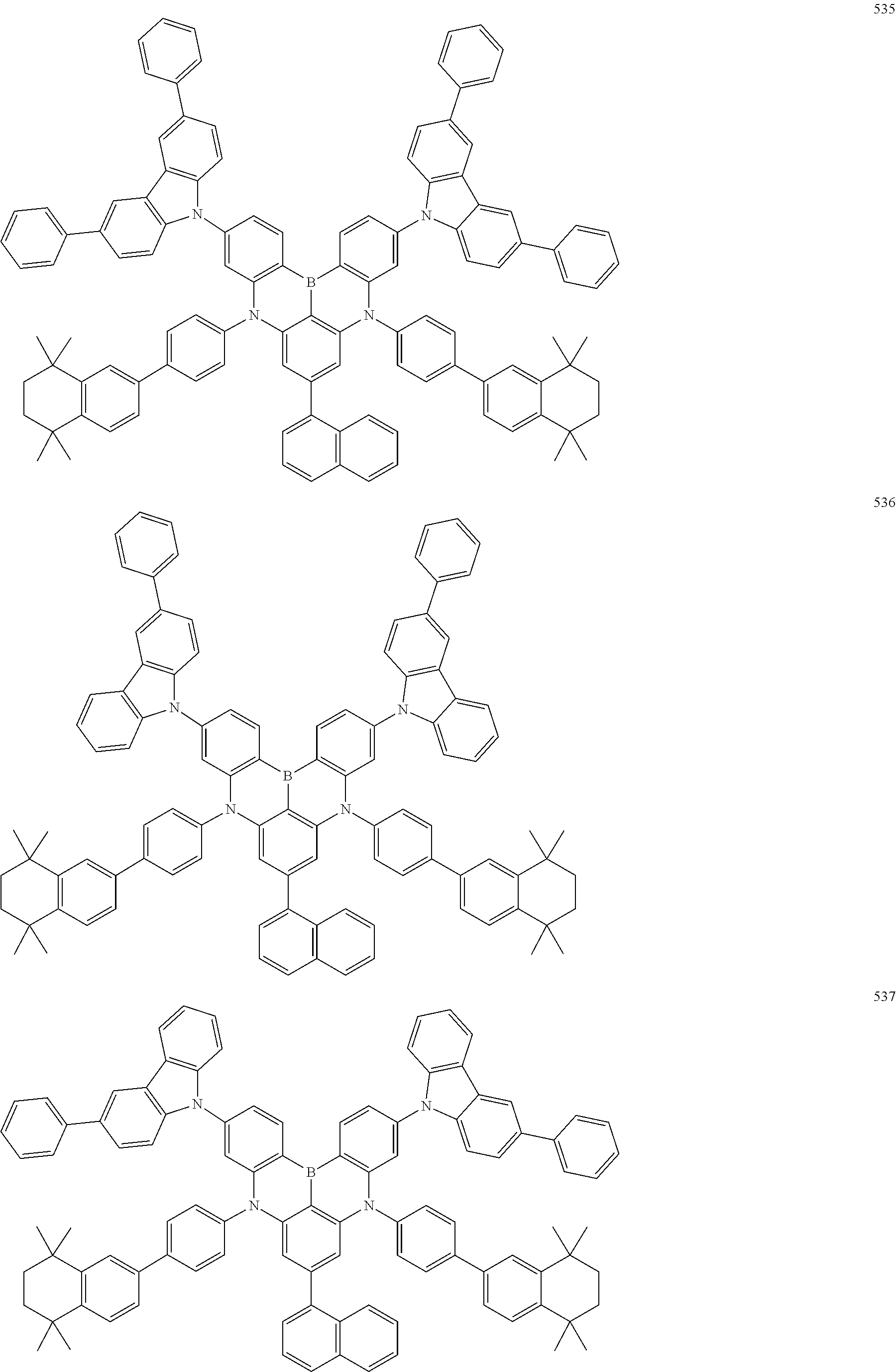

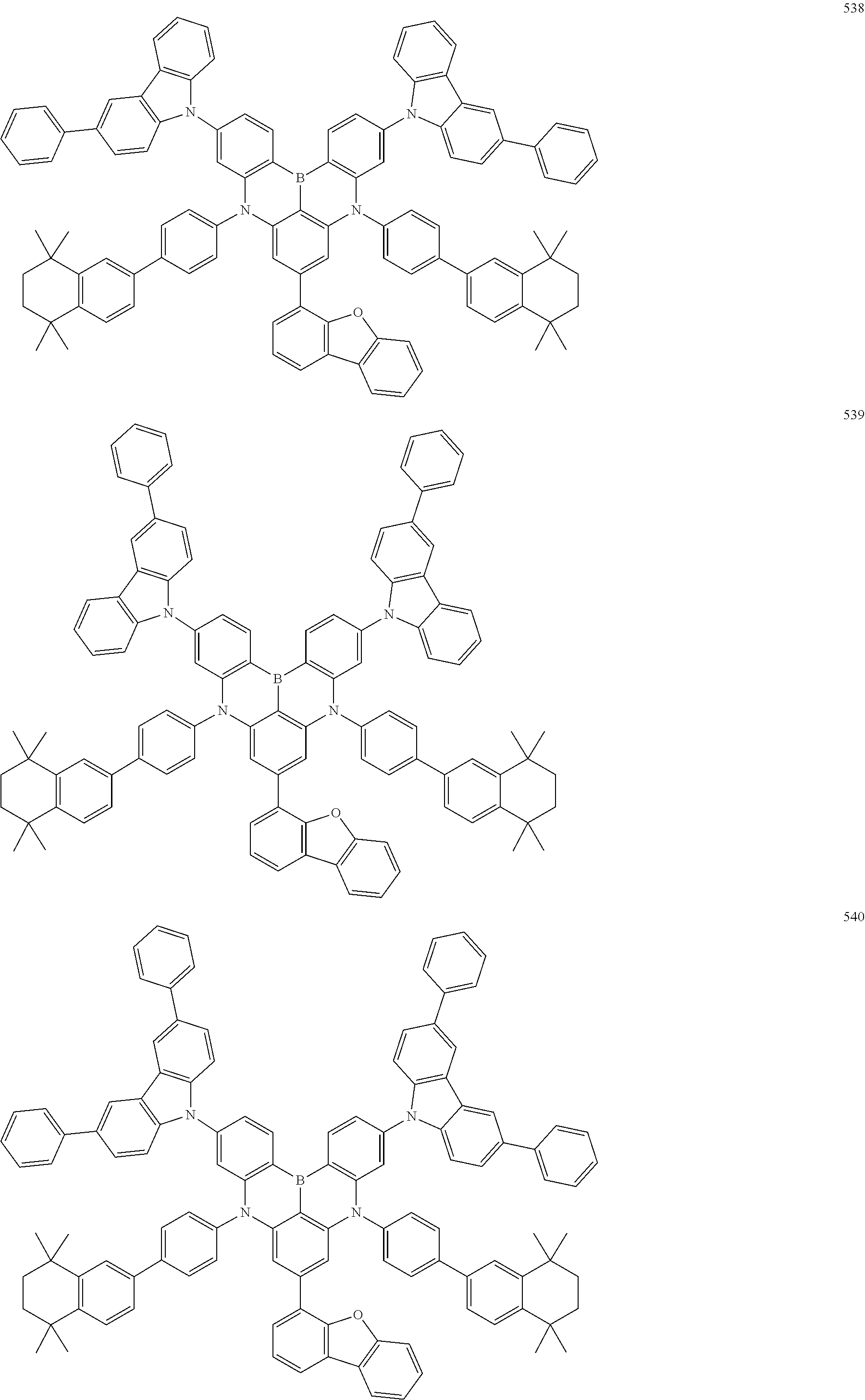

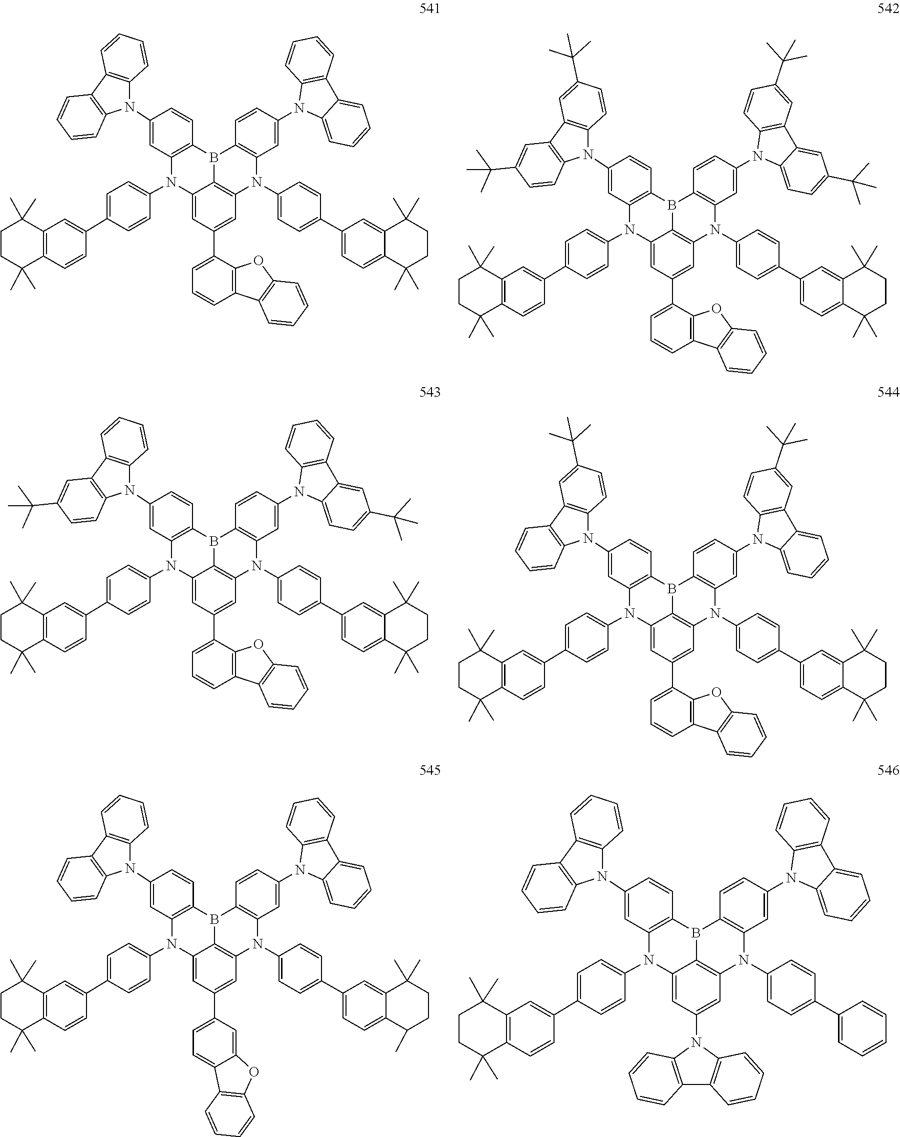

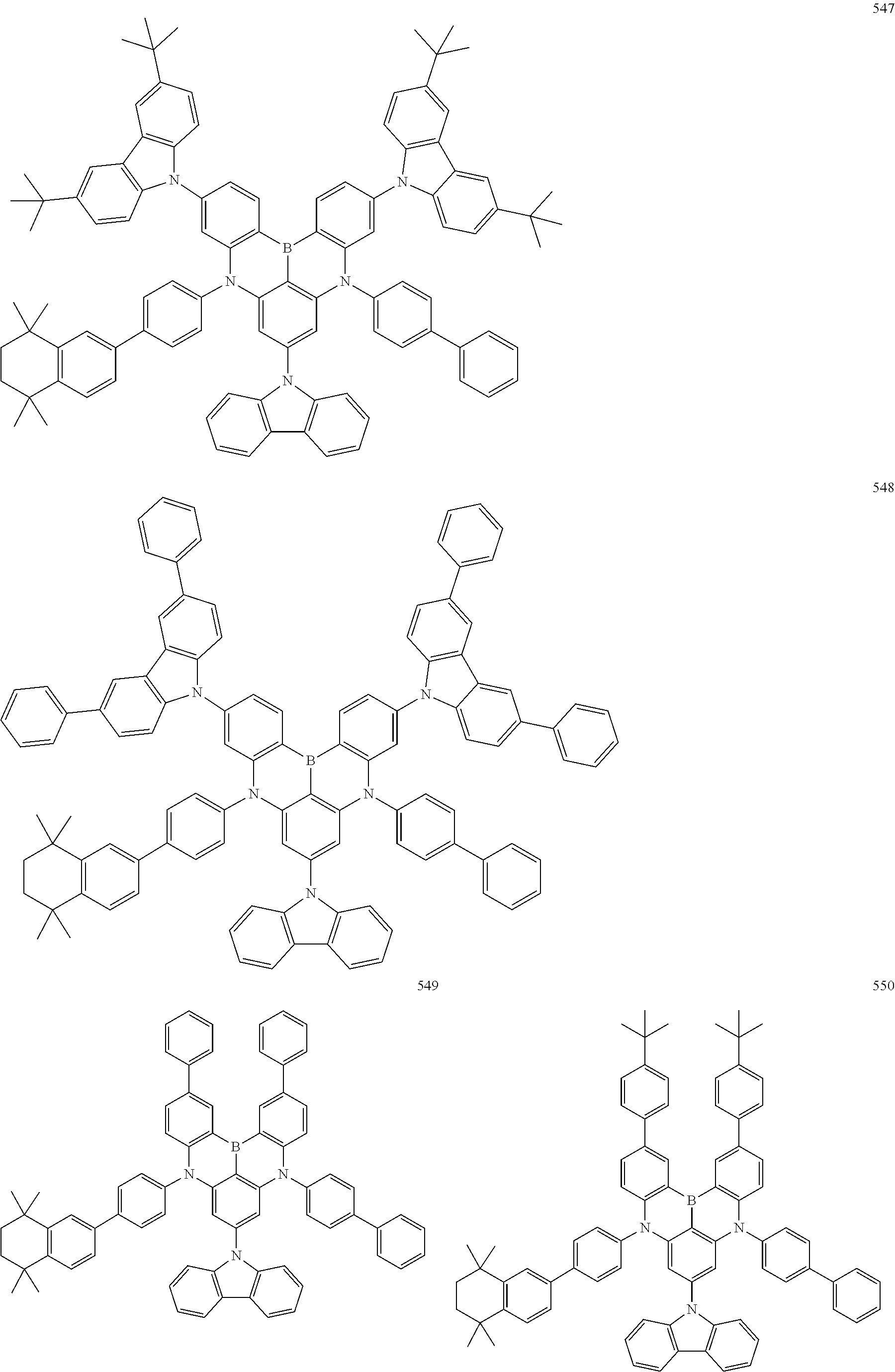

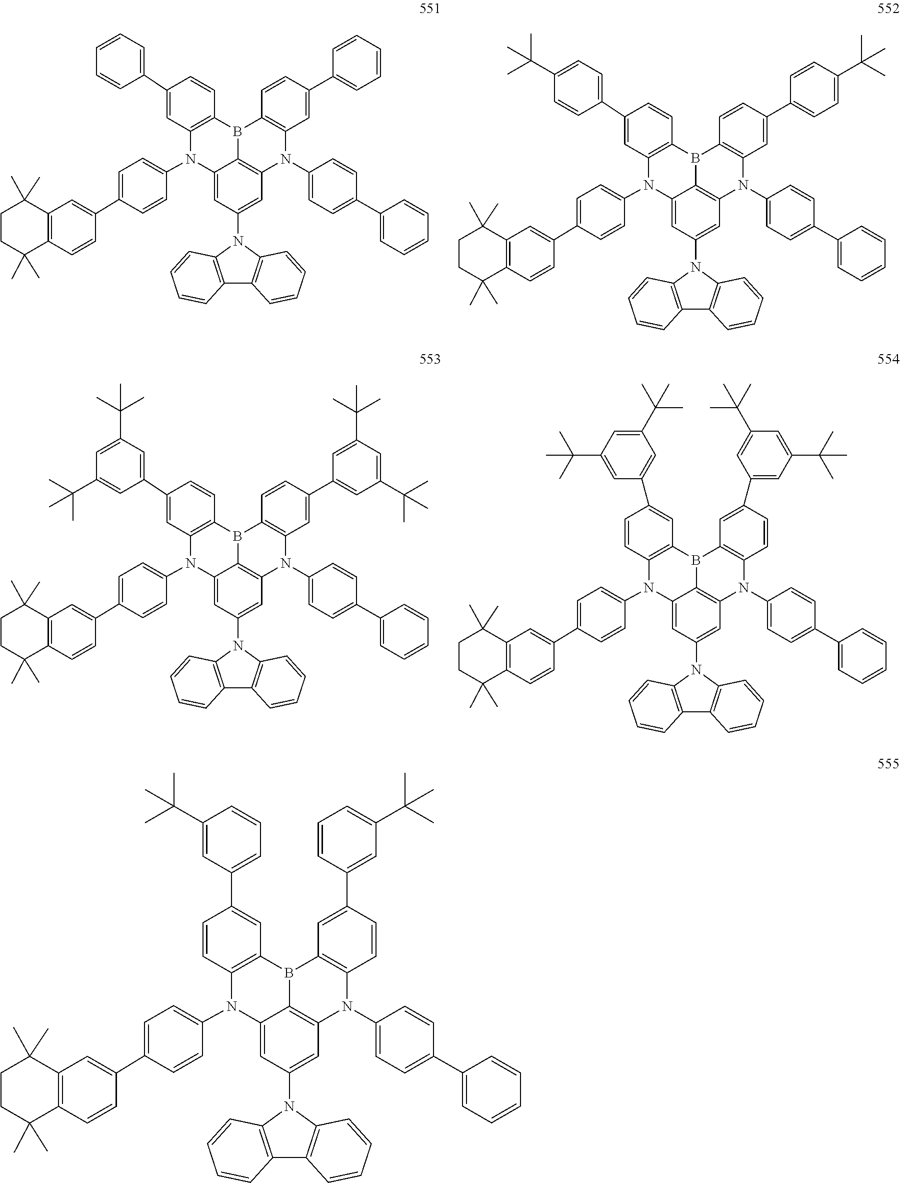

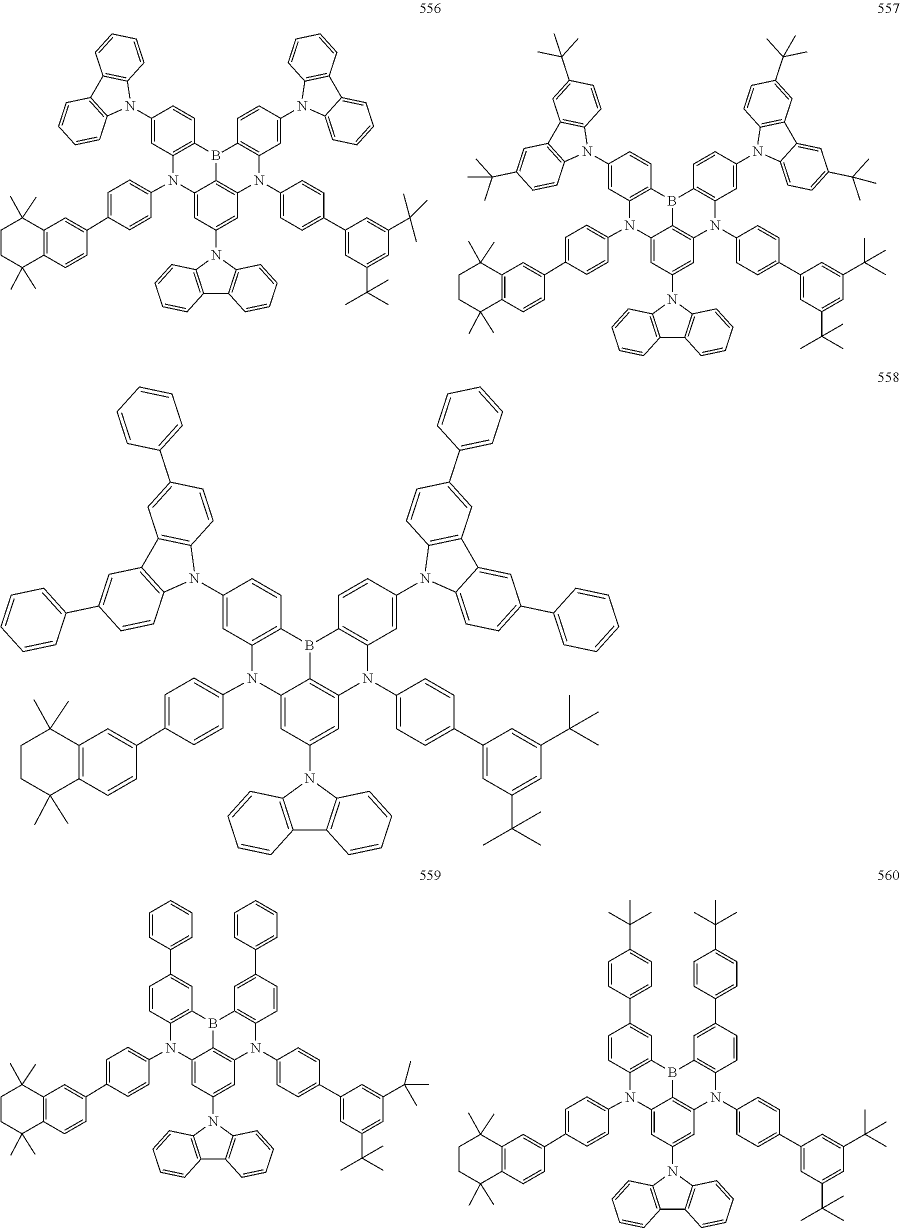

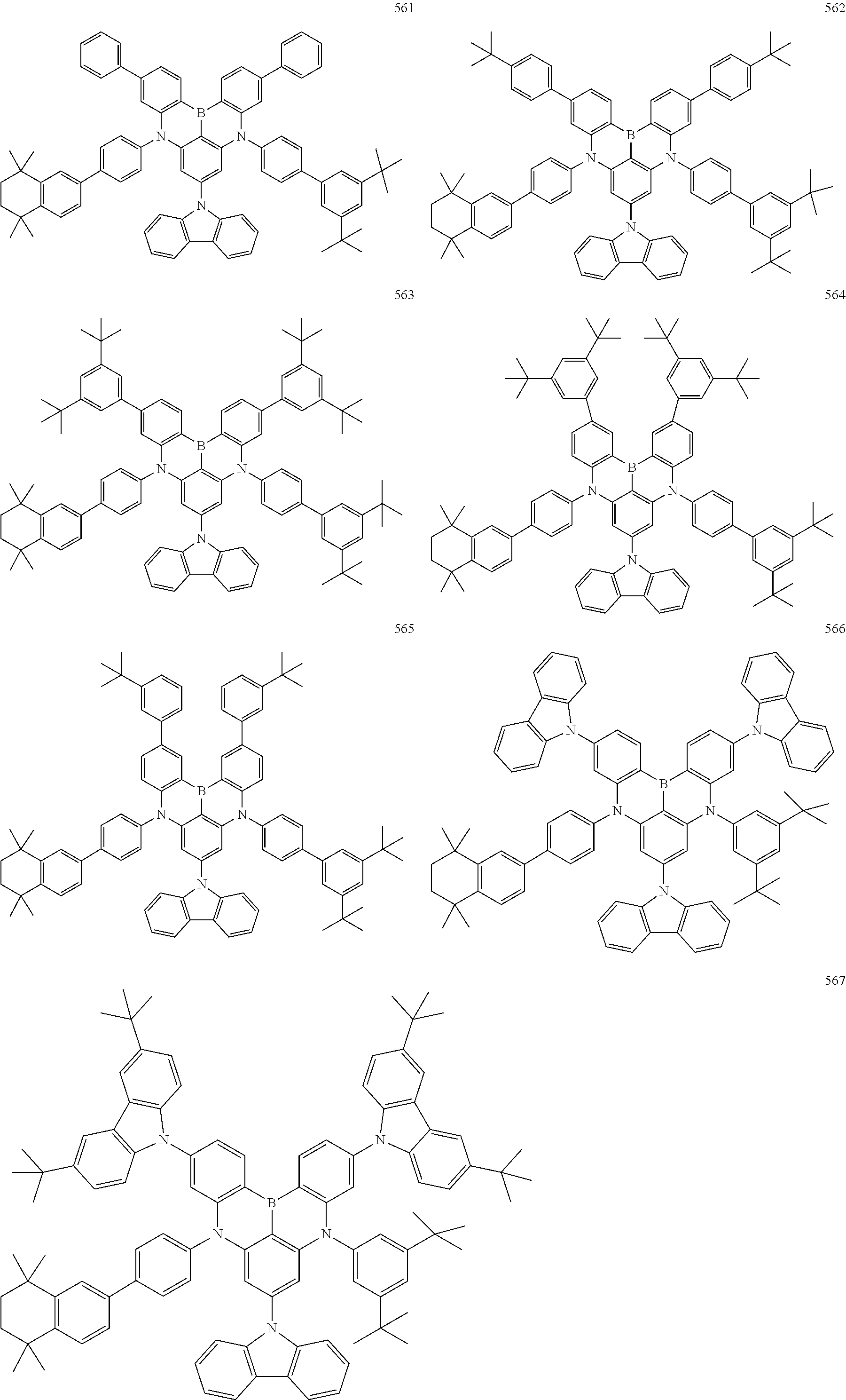

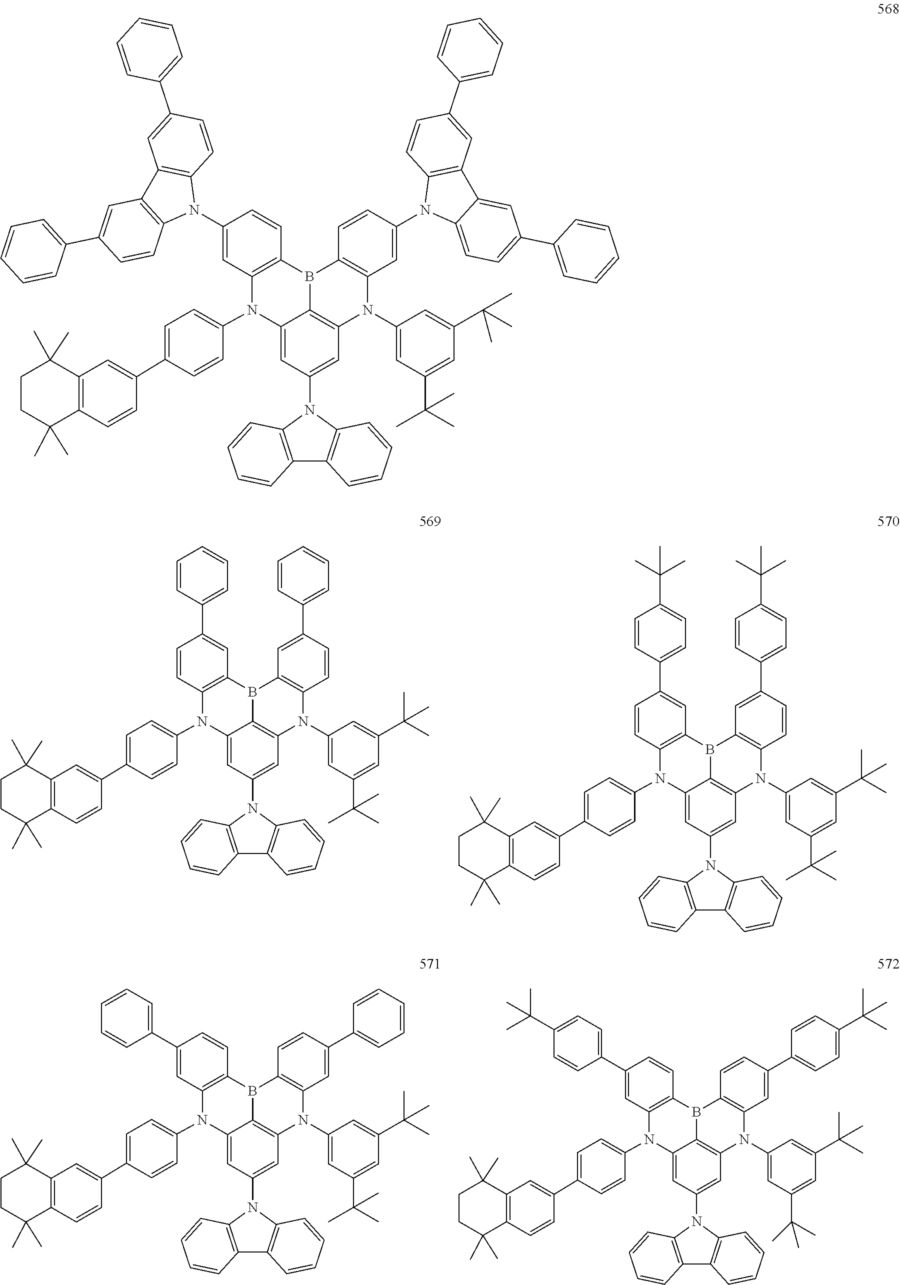

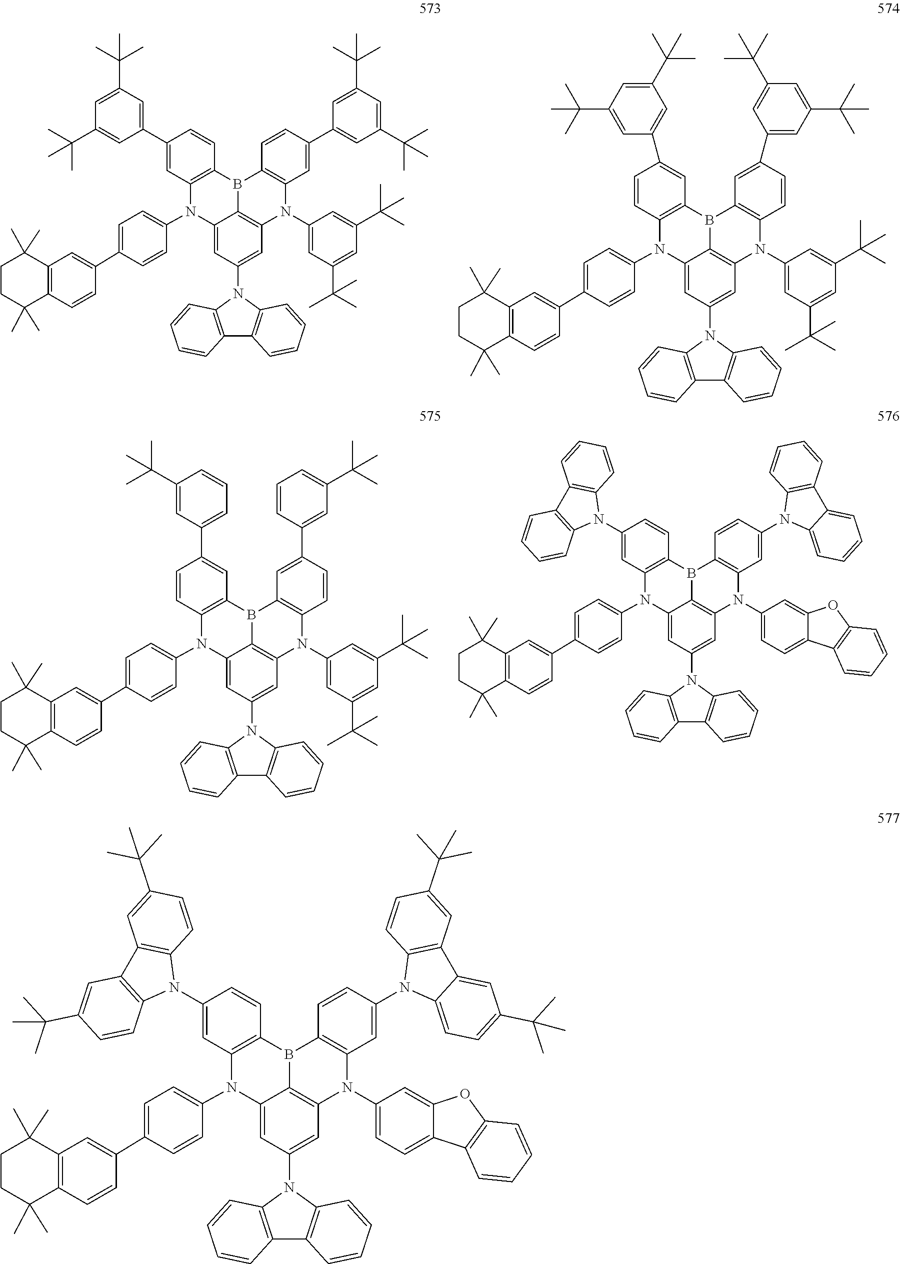

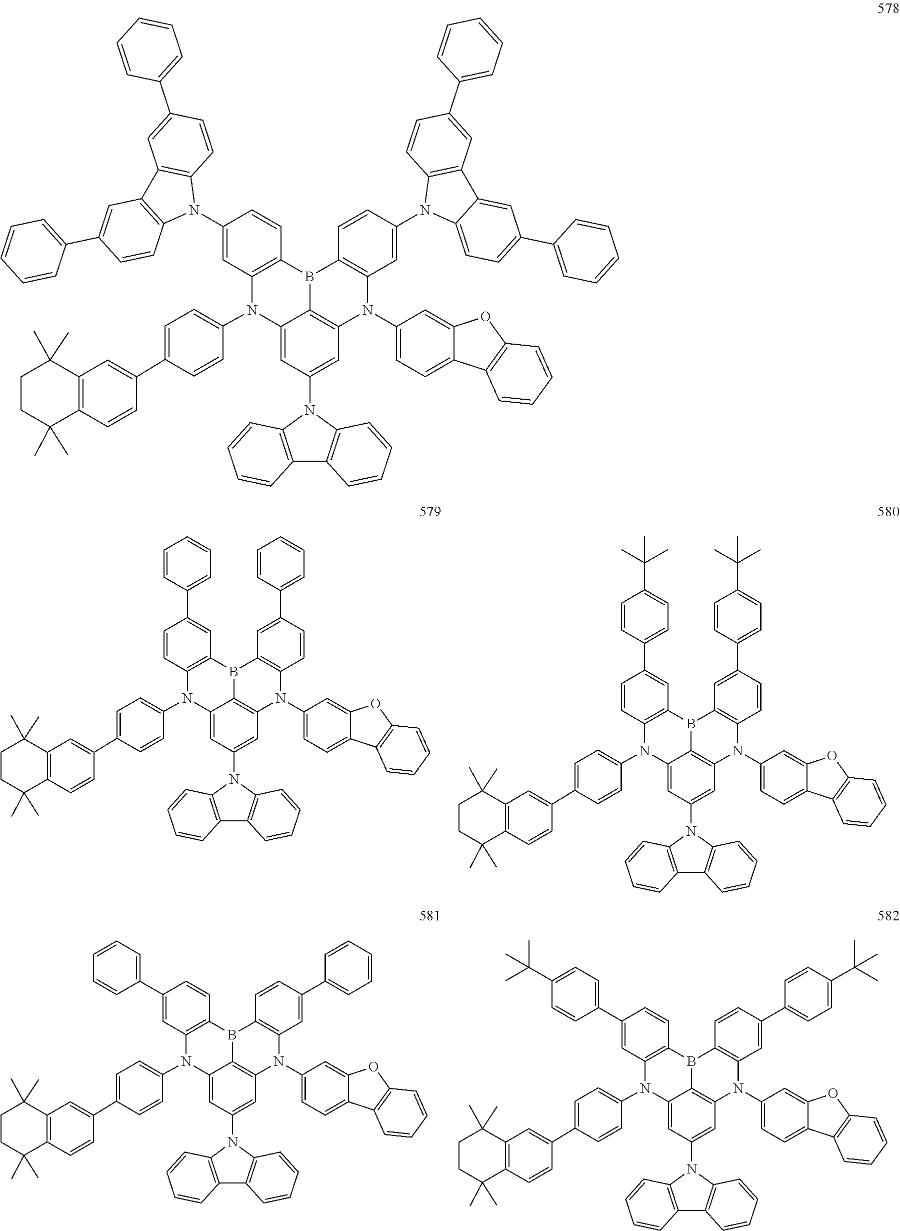

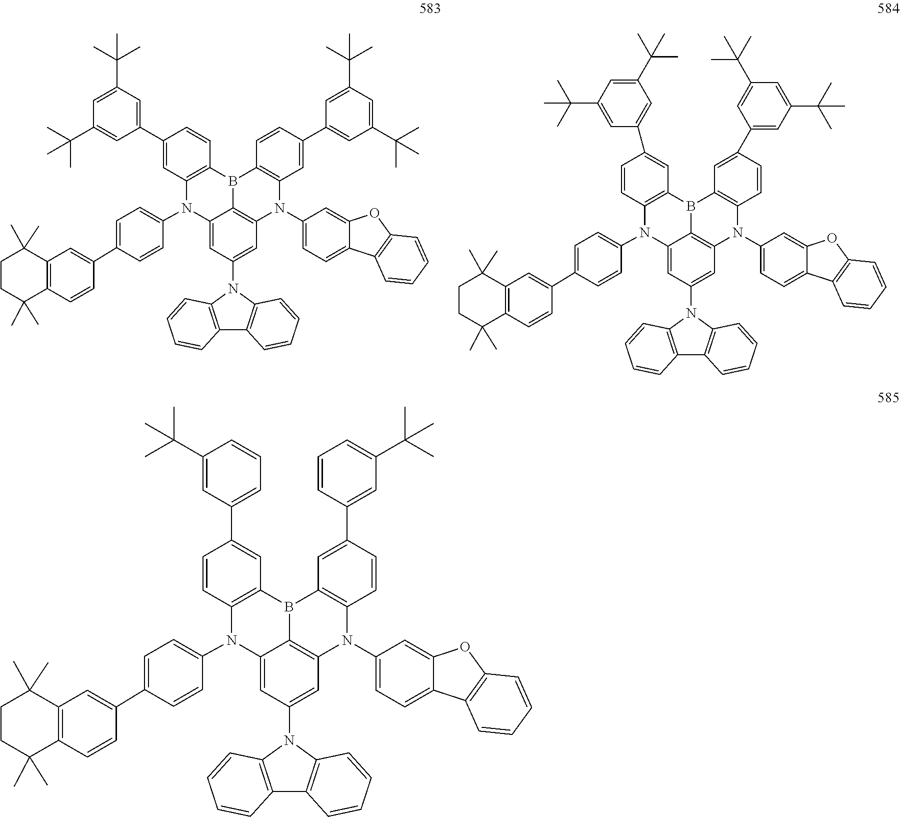

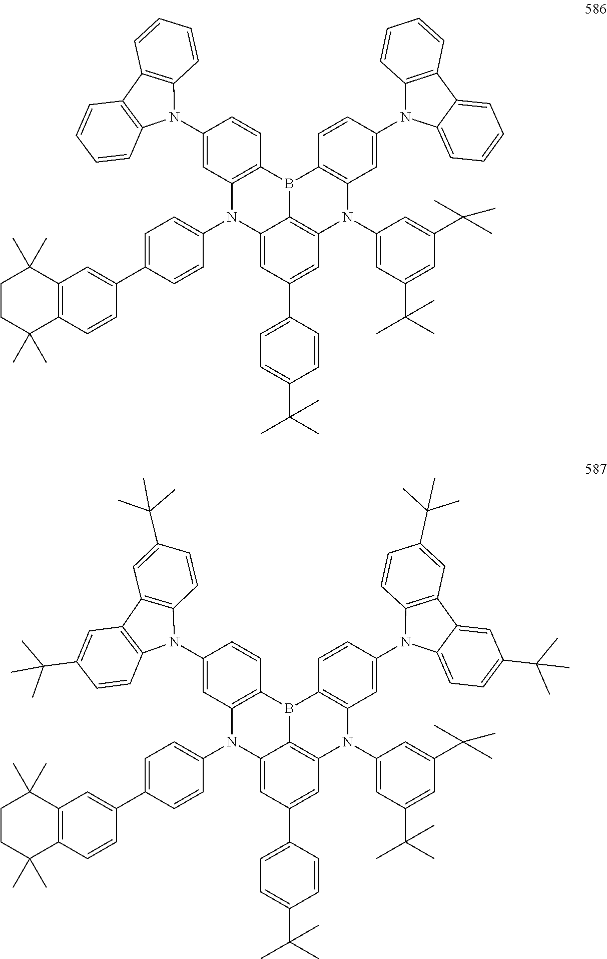 [SMILES: CC(C)(C)c1ccc(-c2cc3c4c(c2)N(c2cc(C(C)(C)C)cc(C(C)(C)C)c2)c2cc(-n5c6ccccc6c6ccccc65)ccc2B4c2ccc(-n4c5ccccc5c5ccccc54)cc2N3c2ccc(-c3ccc4c(c3)C(C)(C)CCC4(C)C)cc2)cc1 ; CC(C)(C)c1ccc(-c2cc3c4c(c2)N(c2cc(C(C)(C)C)cc(C(C)(C)C)c2)c2cc(-n5c6ccc(C(C)(C)C)cc6c6cc(C(C)(C)C)ccc65)ccc2B4c2ccc(-n4c5ccc(C(C)(C)C)cc5c5cc(C(C)(C)C)ccc54)cc2N3c2ccc(-c3ccc4c(c3)C(C)(C)CCC4(C)C)cc2)cc1]

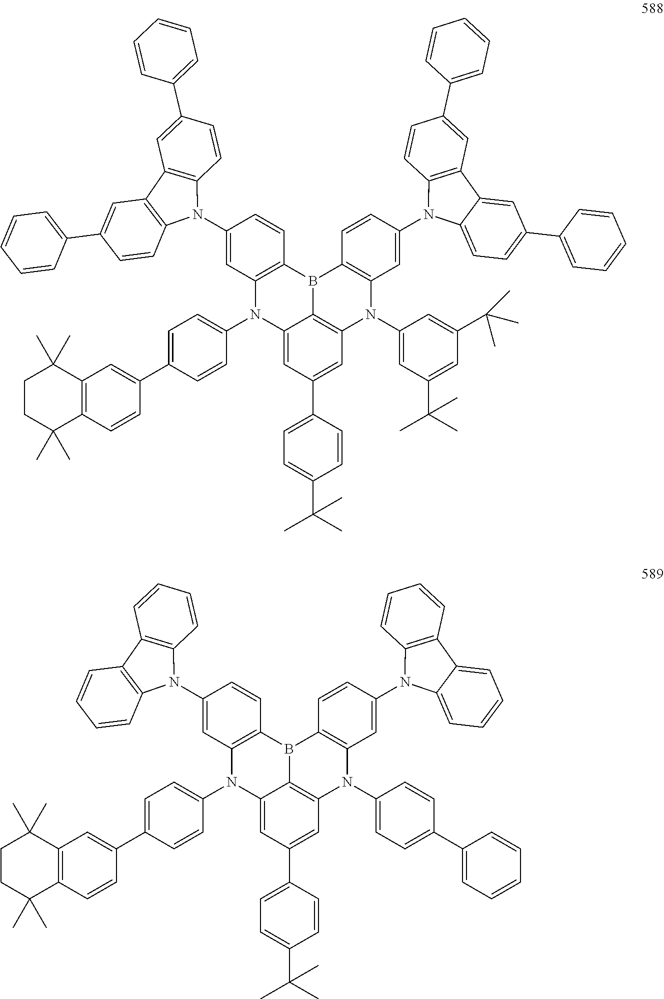 [SMILES: CC(C)(C)c1ccc(-c2cc3c4c(c2)N(c2cc(C(C)(C)C)cc(C(C)(C)C)c2)c2cc(-n5c6ccc(-c7ccccc7)cc6c6cc(-c7ccccc7)ccc65)ccc2B4c2ccc(-n4c5ccc(-c6ccccc6)cc5c5cc(-c6ccccc6)ccc54)cc2N3c2ccc(-c3ccc4c(c3)C(C)(C)CCC4(C)C)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccccc6c6ccccc65)ccc2B4c2ccc(-n4c5ccccc5c5ccccc54)cc2N3c2ccc(-c3ccccc3)cc2)cc1]

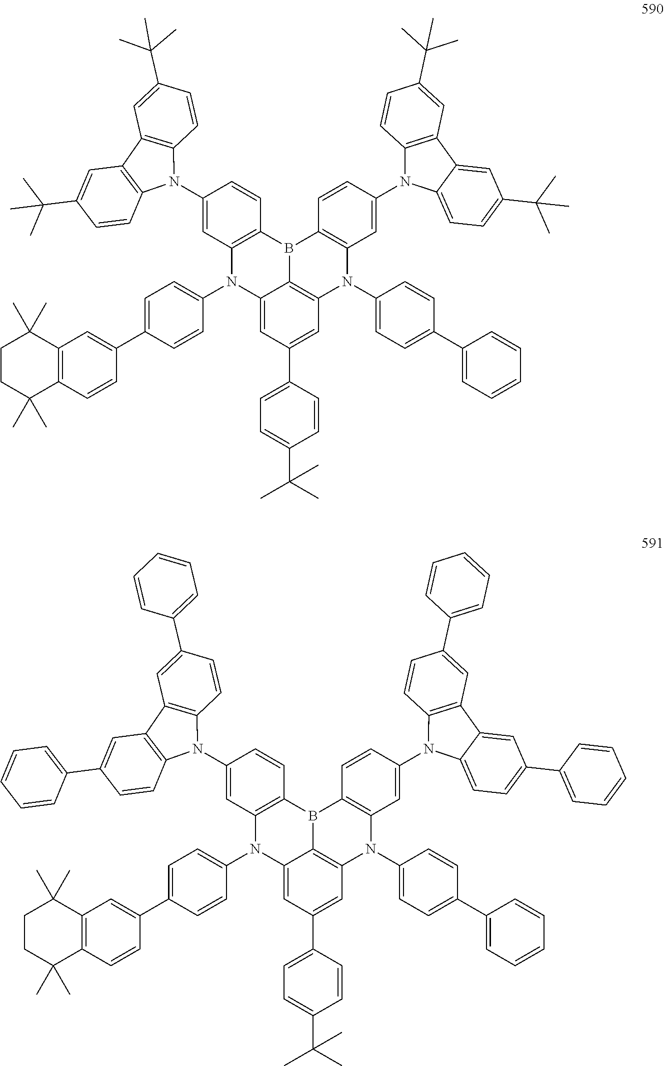 [SMILES: CC(C)(C)c1ccc(-c2cc3c4c(c2)N(c2ccc(-c5ccc6c(c5)C(C)(C)CCC6(C)C)cc2)c2cc(-n5c6ccc(C(C)(C)C)cc6c6cc(C(C)(C)C)ccc65)ccc2B4c2ccc(-n4c5ccc(C(C)(C)C)cc5c5cc(C(C)(C)C)ccc54)cc2N3c2ccc(-c3ccccc3)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2ccc(-n4c5ccc(-c6ccccc6)cc5c5cc(-c6ccccc6)ccc54)cc2N3c2ccc(-c3ccccc3)cc2)cc1]

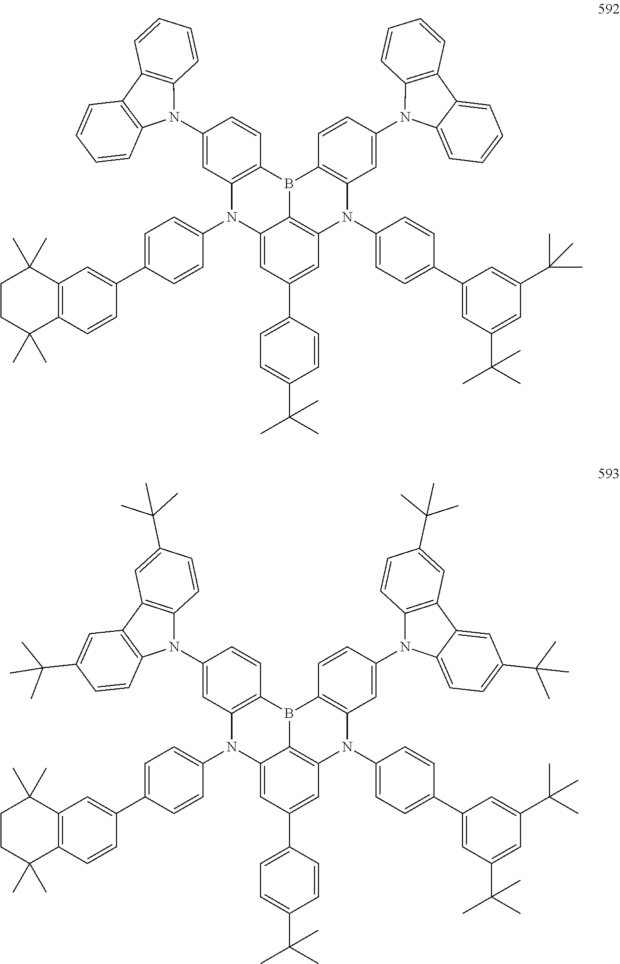 [SMILES: CC(C)(C)c1ccc(-c2cc3c4c(c2)N(c2ccc(-c5ccc6c(c5)C(C)(C)CCC6(C)C)cc2)c2cc(-n5c6ccccc6c6ccccc65)ccc2B4c2ccc(-n4c5ccccc5c5ccccc54)cc2N3c2ccc(-c3cc(C(C)(C)C)cc(C(C)(C)C)c3)cc2)cc1 ; CC(C)(C)c1ccc(-c2cc3c4c(c2)N(c2ccc(-c5ccc6c(c5)C(C)(C)CCC6(C)C)cc2)c2cc(-n5c6ccc(C(C)(C)C)cc6c6cc(C(C)(C)C)ccc65)ccc2B4c2ccc(-n4c5ccc(C(C)(C)C)cc5c5cc(C(C)(C)C)ccc54)cc2N3c2ccc(-c3cc(C(C)(C)C)cc(C(C)(C)C)c3)cc2)cc1]

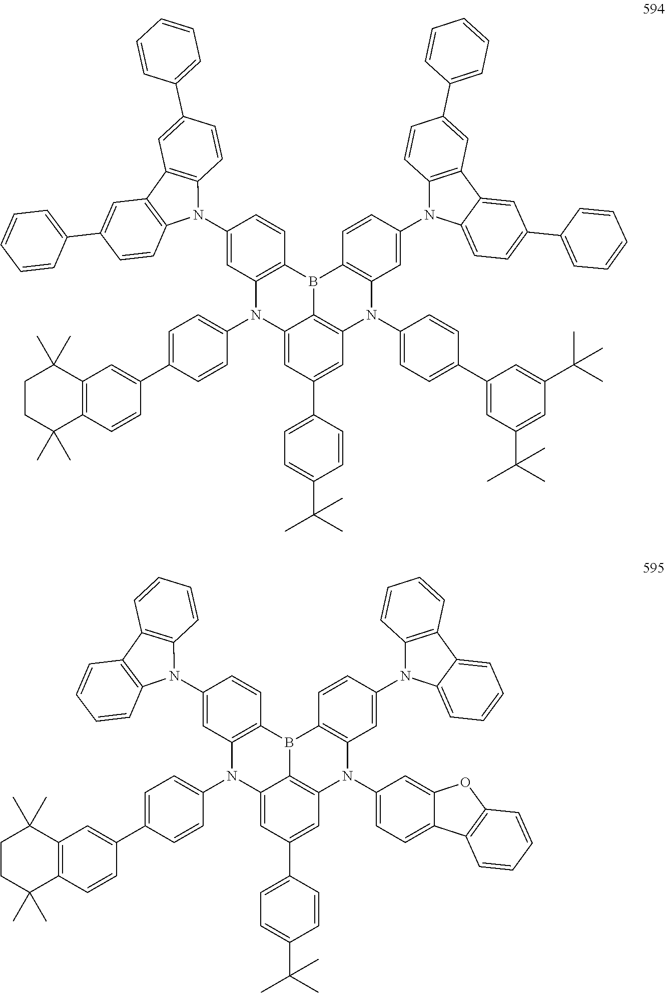 [SMILES: CC(C)(C)c1ccc(-c2cc3c4c(c2)N(c2ccc(-c5ccc6c(c5)C(C)(C)CCC6(C)C)cc2)c2cc(-n5c6ccc(-c7ccccc7)cc6c6cc(-c7ccccc7)ccc65)ccc2B4c2ccc(-n4c5ccc(-c6ccccc6)cc5c5cc(-c6ccccc6)ccc54)cc2N3c2ccc(-c3cc(C(C)(C)C)cc(C(C)(C)C)c3)cc2)cc1 ; CC(C)(C)c1ccc(-c2cc3c4c(c2)N(c2ccc5c(c2)oc2ccccc25)c2cc(-n5c6ccccc6c6ccccc65)ccc2B4c2ccc(-n4c5ccccc5c5ccccc54)cc2N3c2ccc(-c3ccc4c(c3)C(C)(C)CCC4(C)C)cc2)cc1]

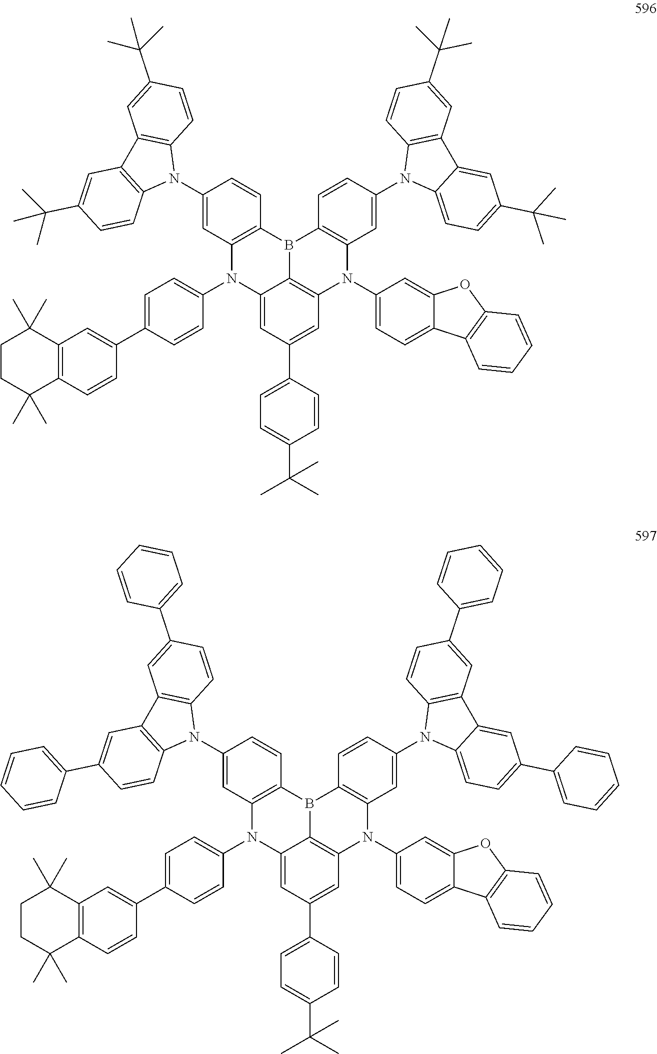 [SMILES: CC(C)(C)c1ccc(-c2cc3c4c(c2)N(c2ccc5c(c2)oc2ccccc25)c2cc(-n5c6ccc(C(C)(C)C)cc6c6cc(C(C)(C)C)ccc65)ccc2B4c2ccc(-n4c5ccc(C(C)(C)C)cc5c5cc(C(C)(C)C)ccc54)cc2N3c2ccc(-c3ccc4c(c3)C(C)(C)CCC4(C)C)cc2)cc1 ; CC(C)(C)c1ccc(-c2cc3c4c(c2)N(c2ccc5c(c2)oc2ccccc25)c2cc(-n5c6ccc(-c7ccccc7)cc6c6cc(-c7ccccc7)ccc65)ccc2B4c2ccc(-n4c5ccc(-c6ccccc6)cc5c5cc(-c6ccccc6)ccc54)cc2N3c2ccc(-c3ccc4c(c3)C(C)(C)CCC4(C)C)cc2)cc1]

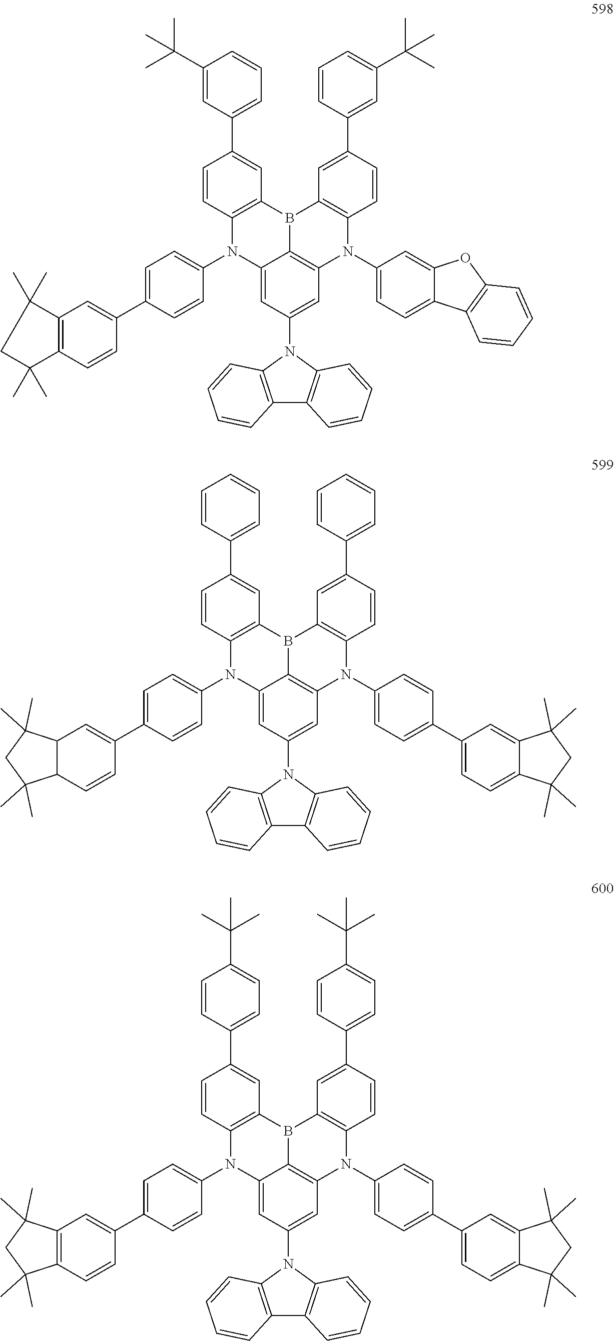

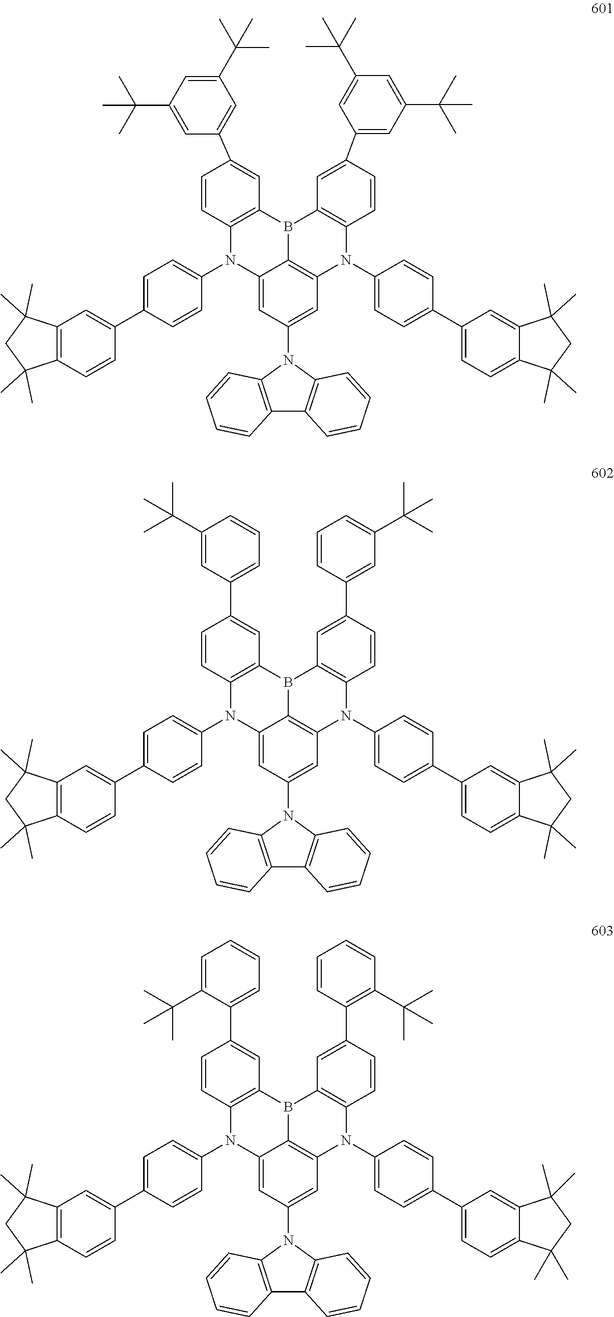

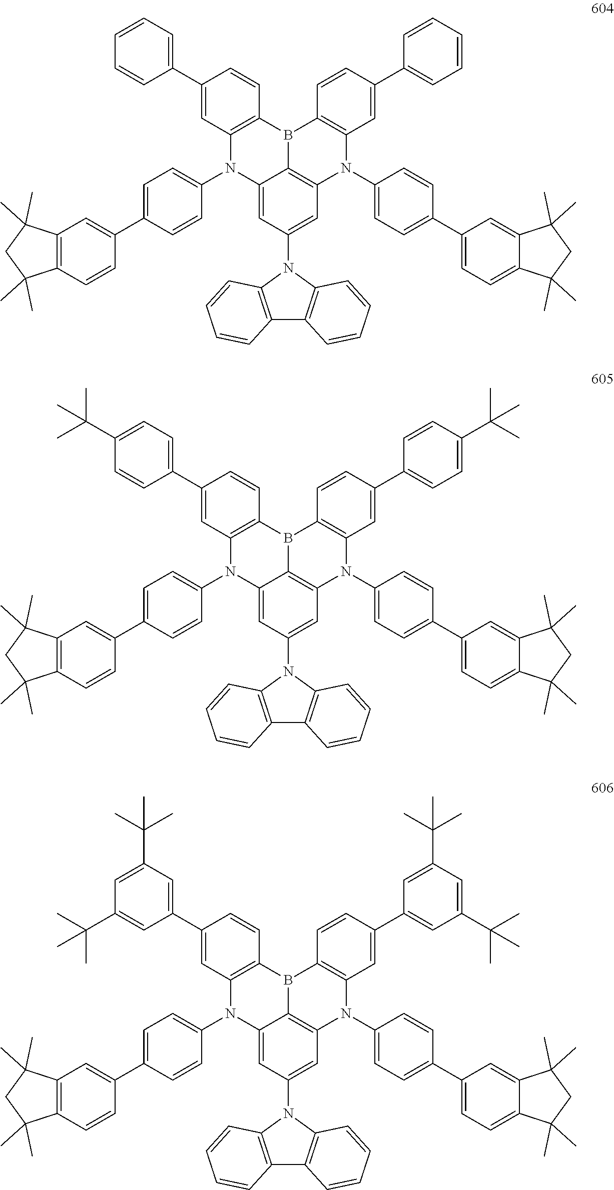

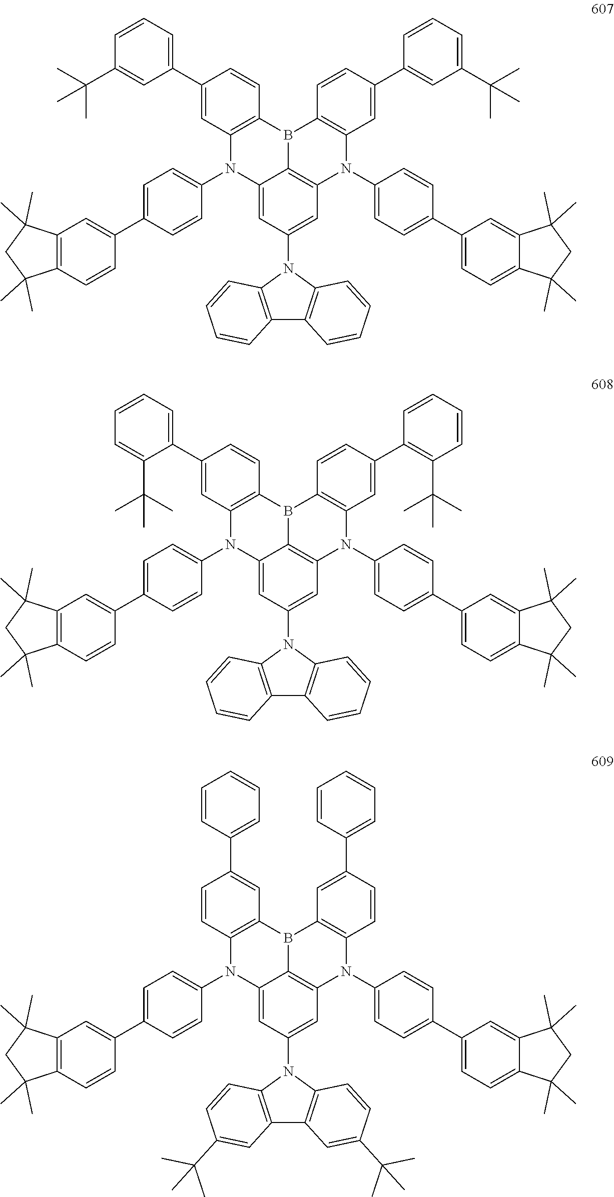

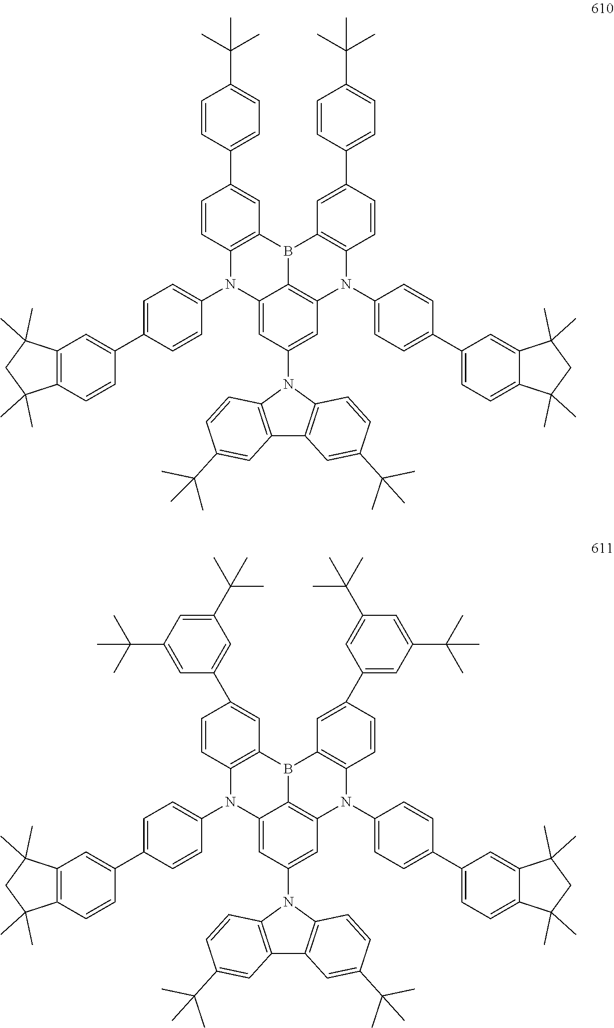

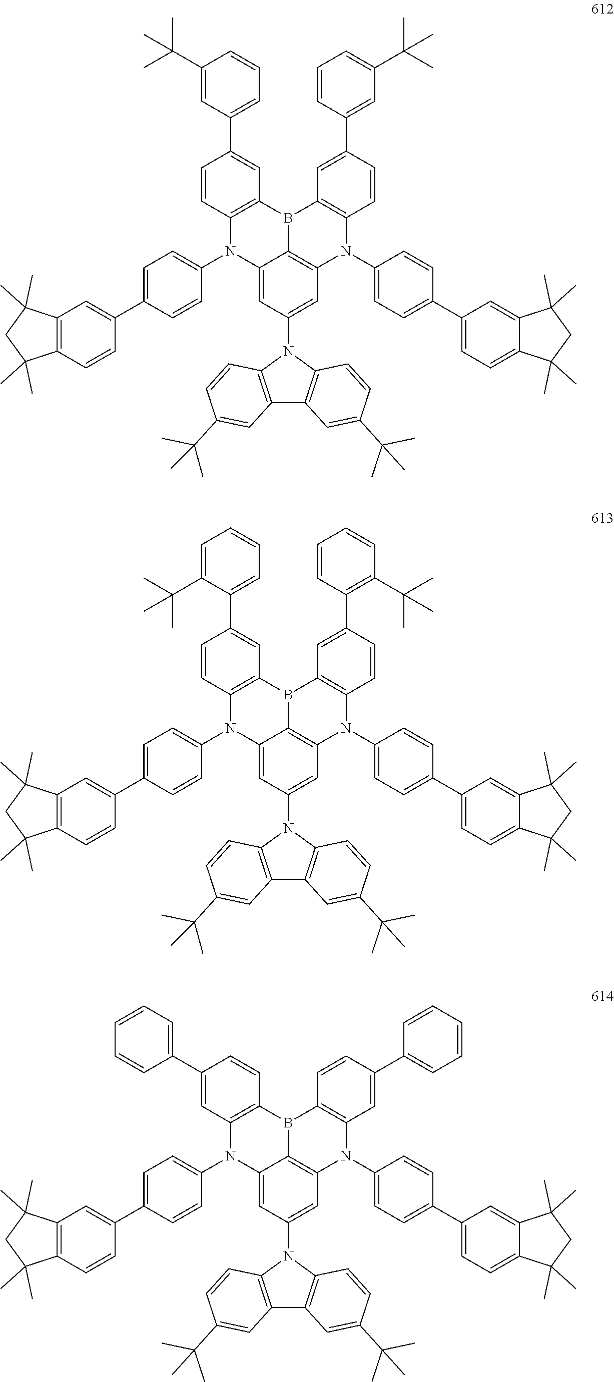

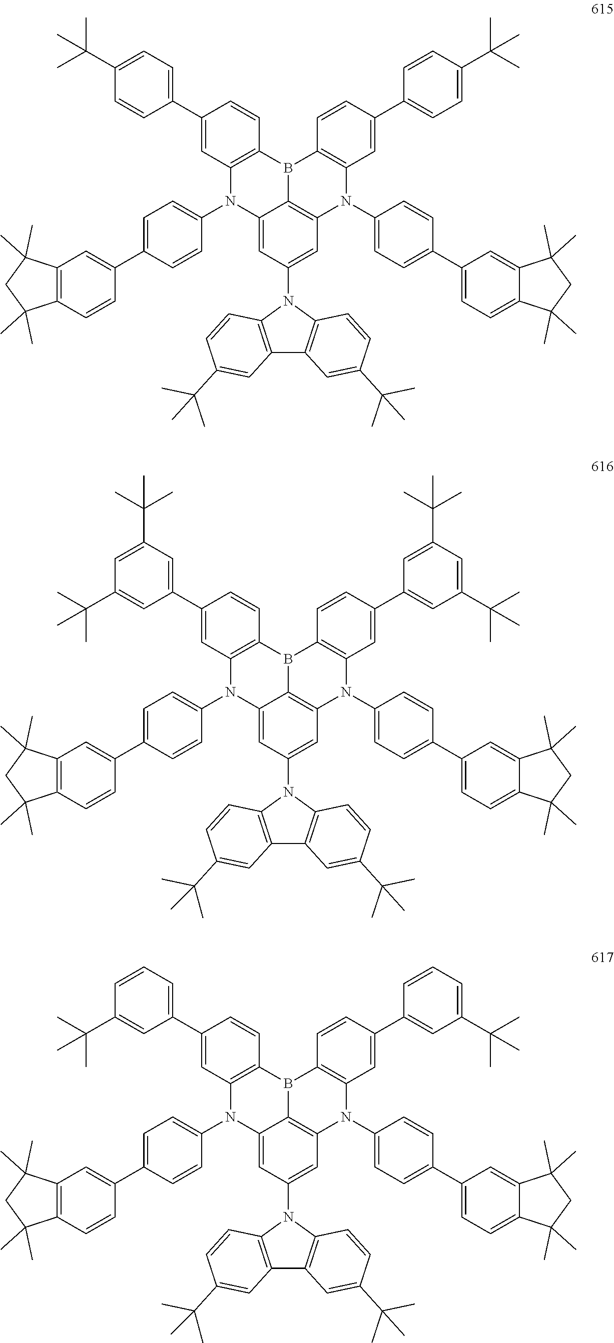

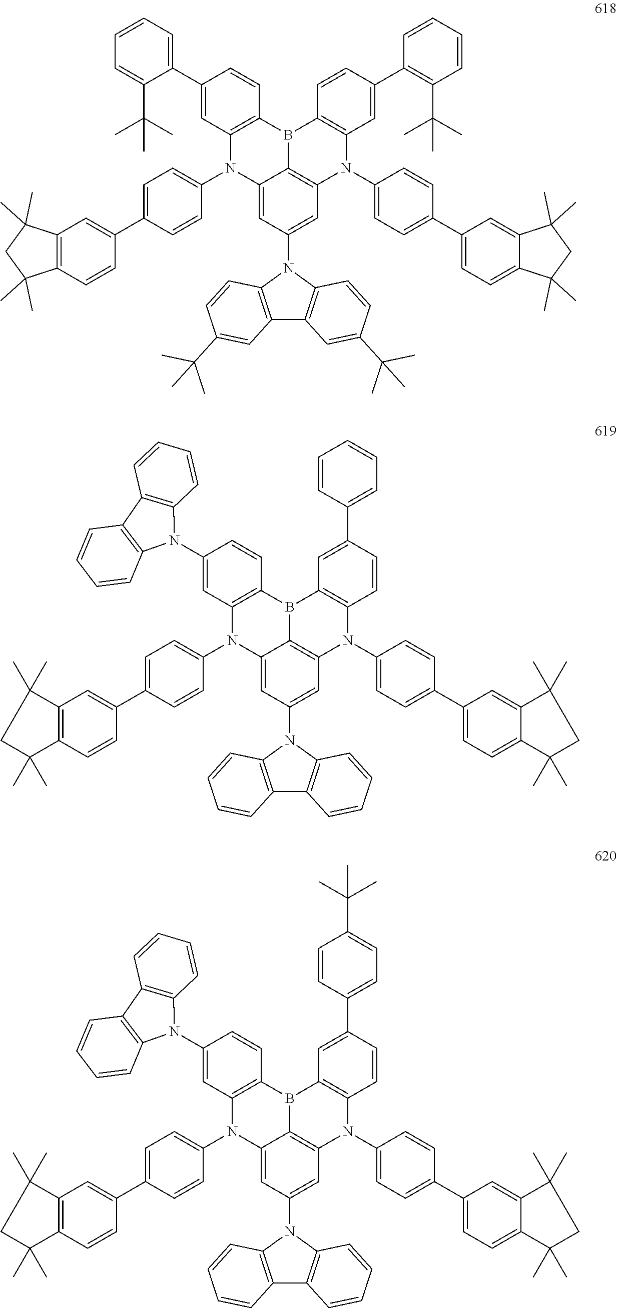

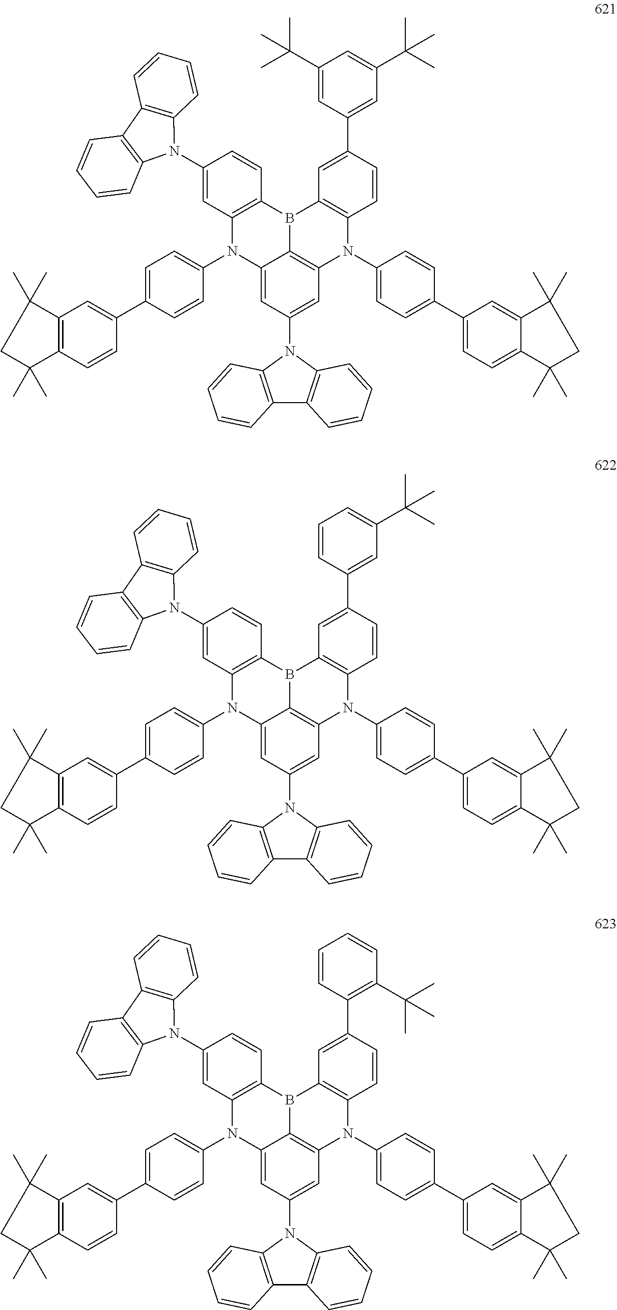

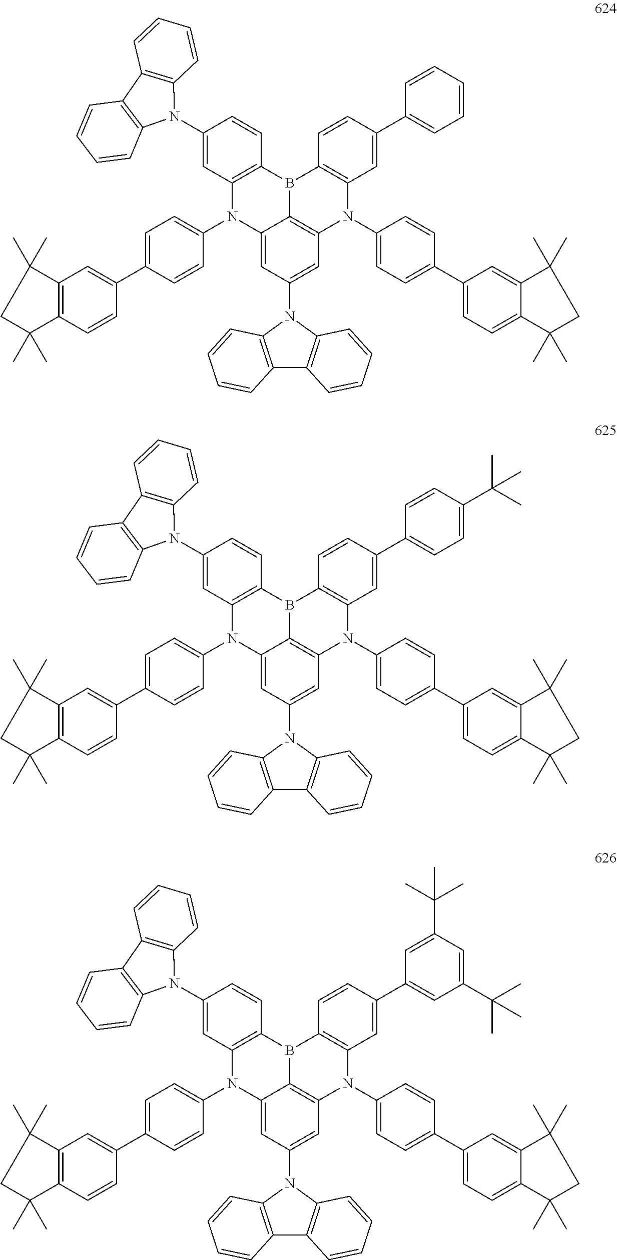

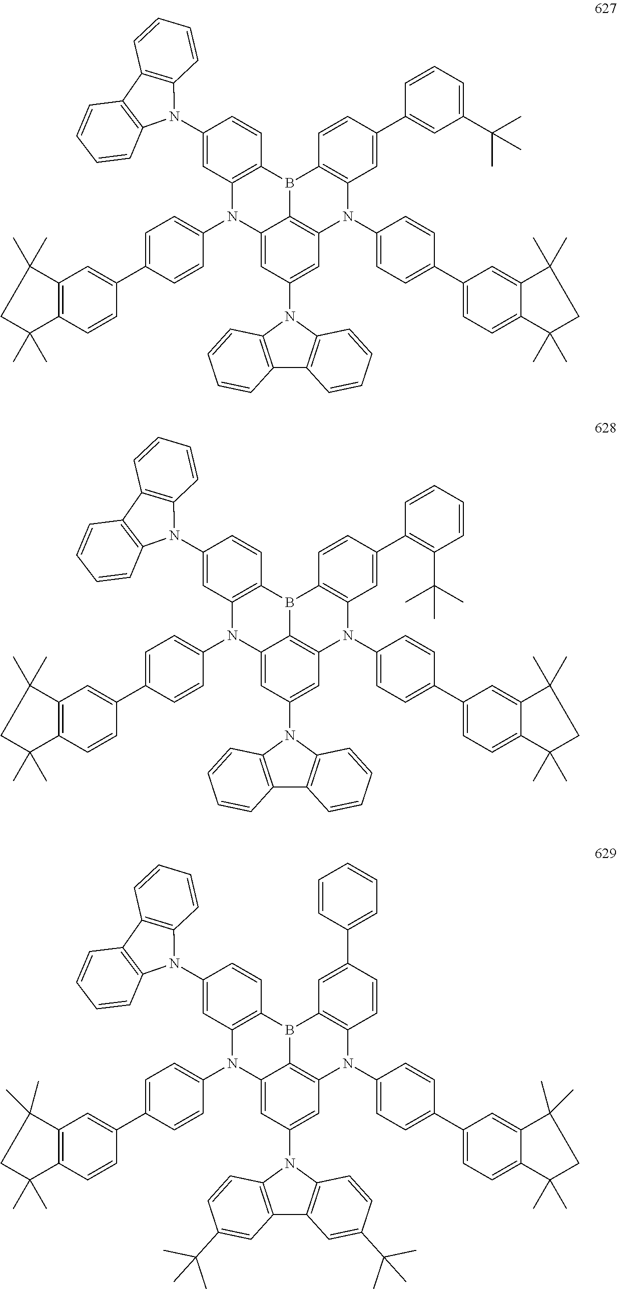

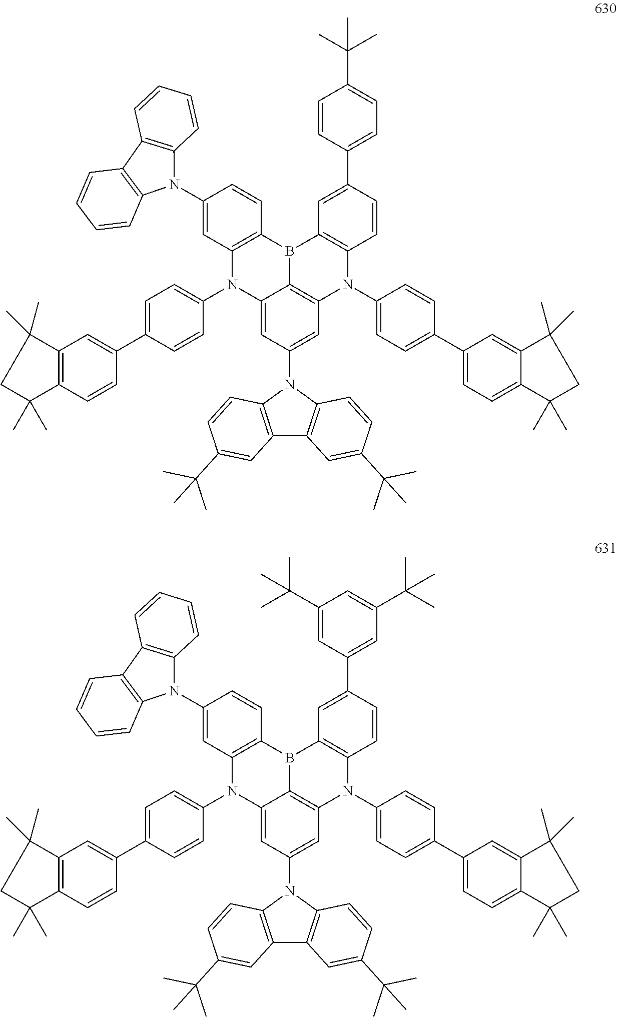 [SMILES: CC(C)(C)c1ccc(-c2ccc3c(c2)B2c4ccc(-n5c6ccccc6c6ccccc65)cc4N(c4ccc(-c5ccc6c(c5)C(C)(C)CC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CC4(C)C)cc2)cc1 ; CC(C)(C)c1cc(-c2ccc3c(c2)B2c4ccc(-n5c6ccccc6c6ccccc65)cc4N(c4ccc(-c5ccc6c(c5)C(C)(C)CC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CC4(C)C)cc2)cc(C(C)(C)C)c1]

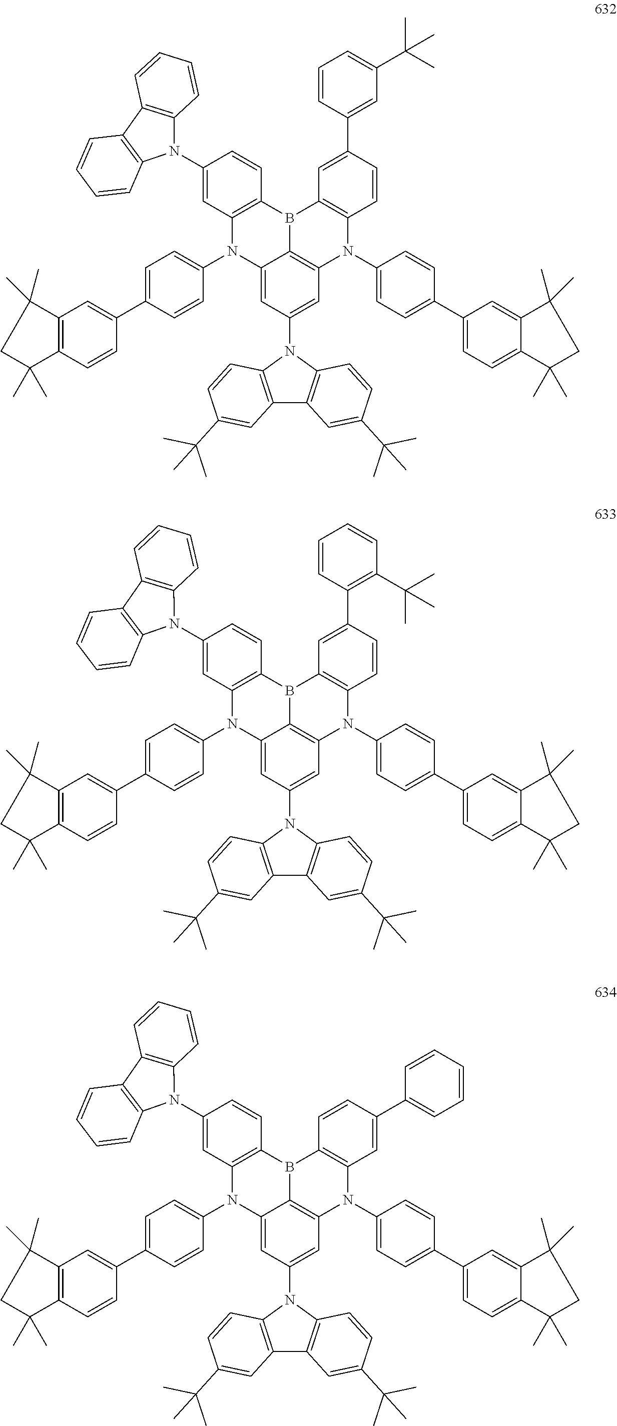

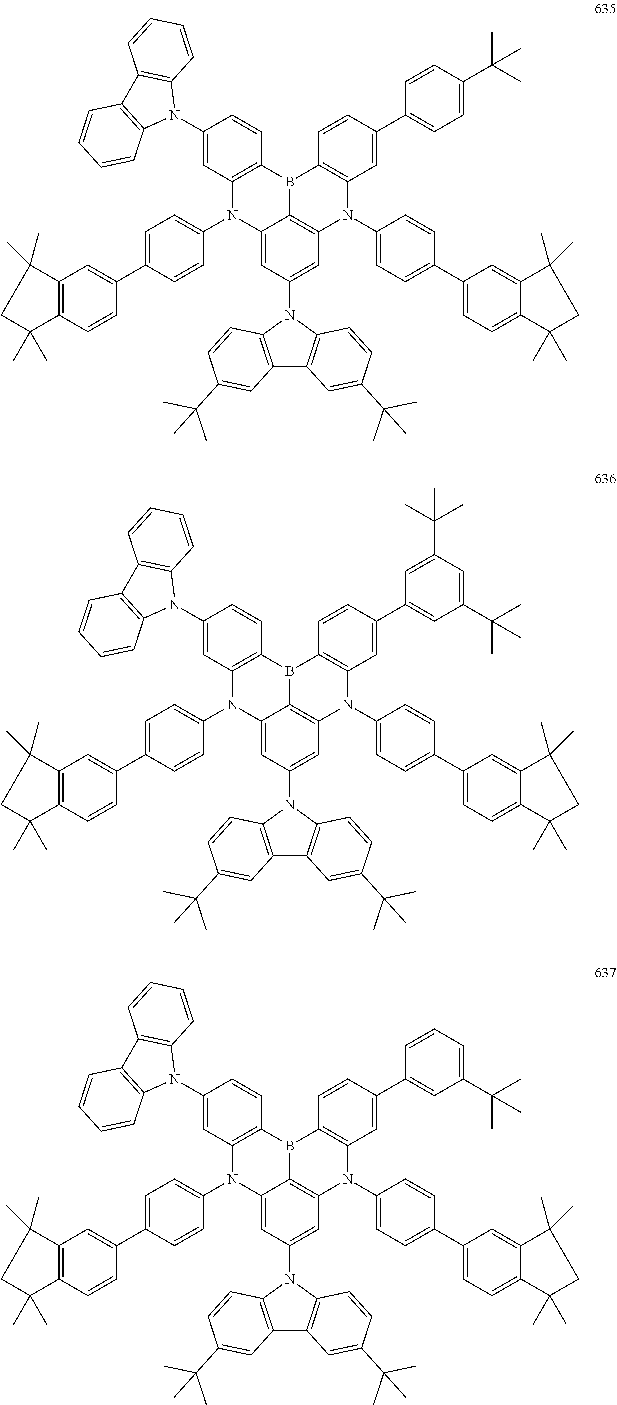

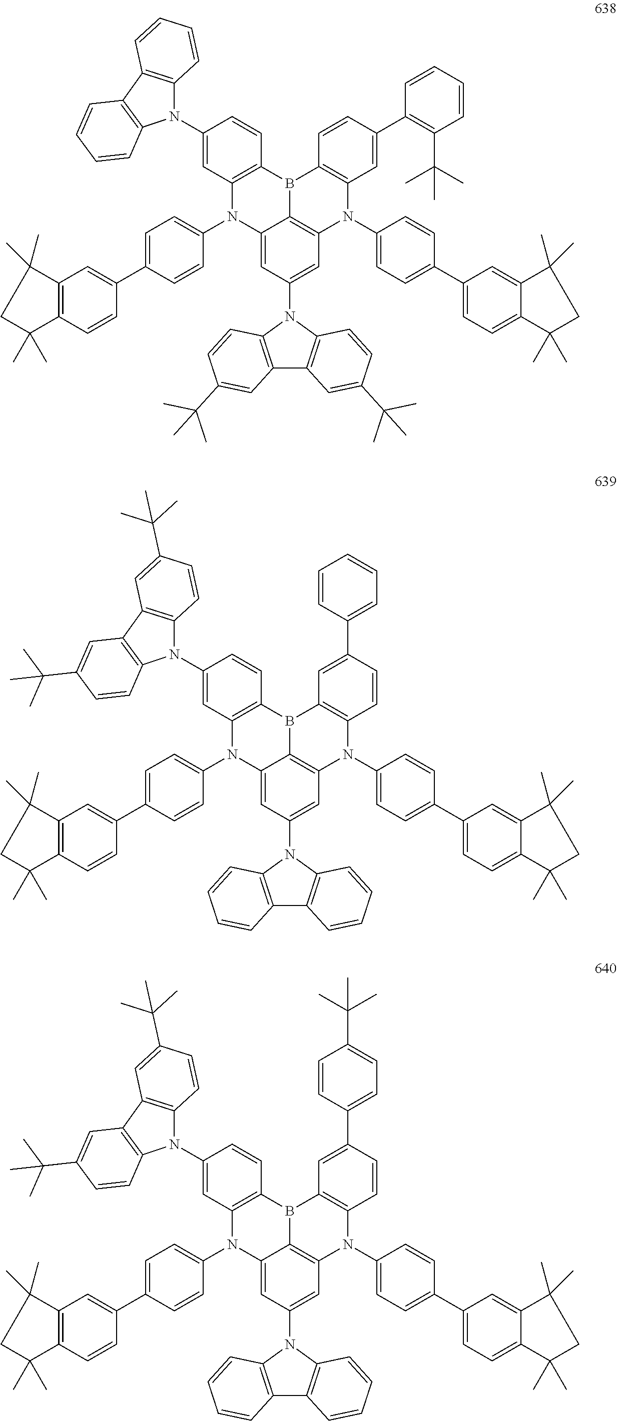

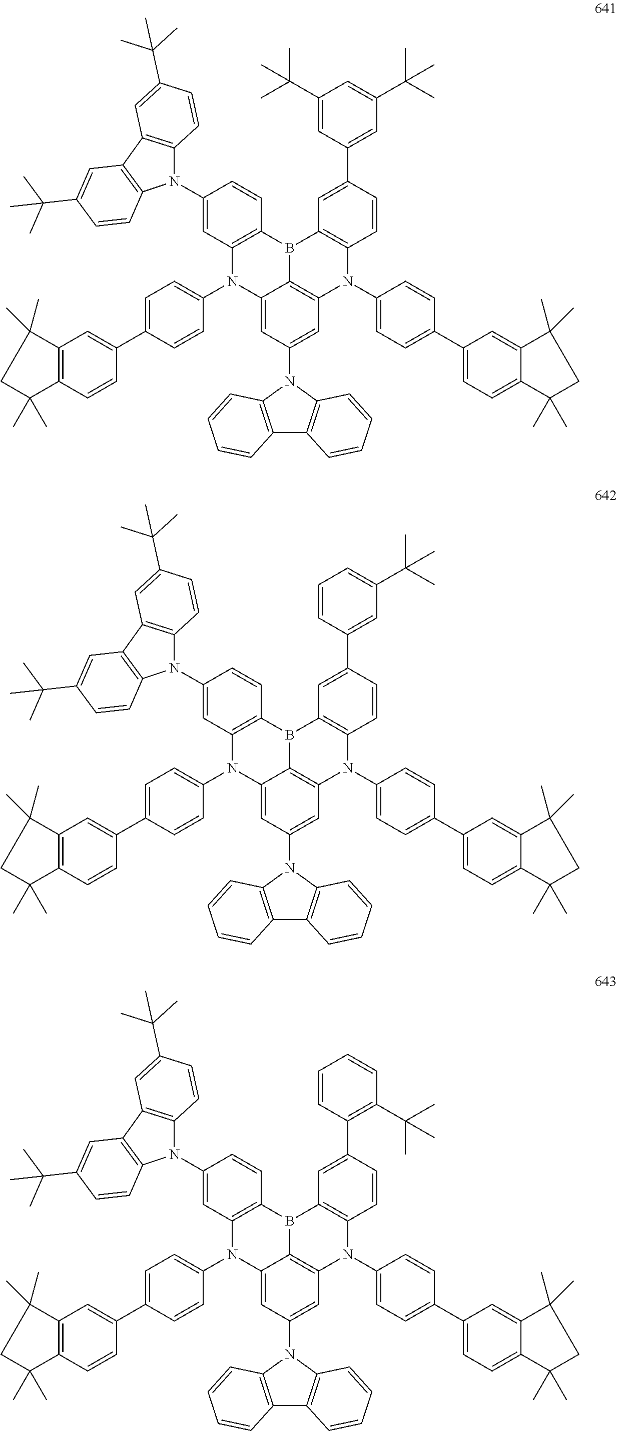

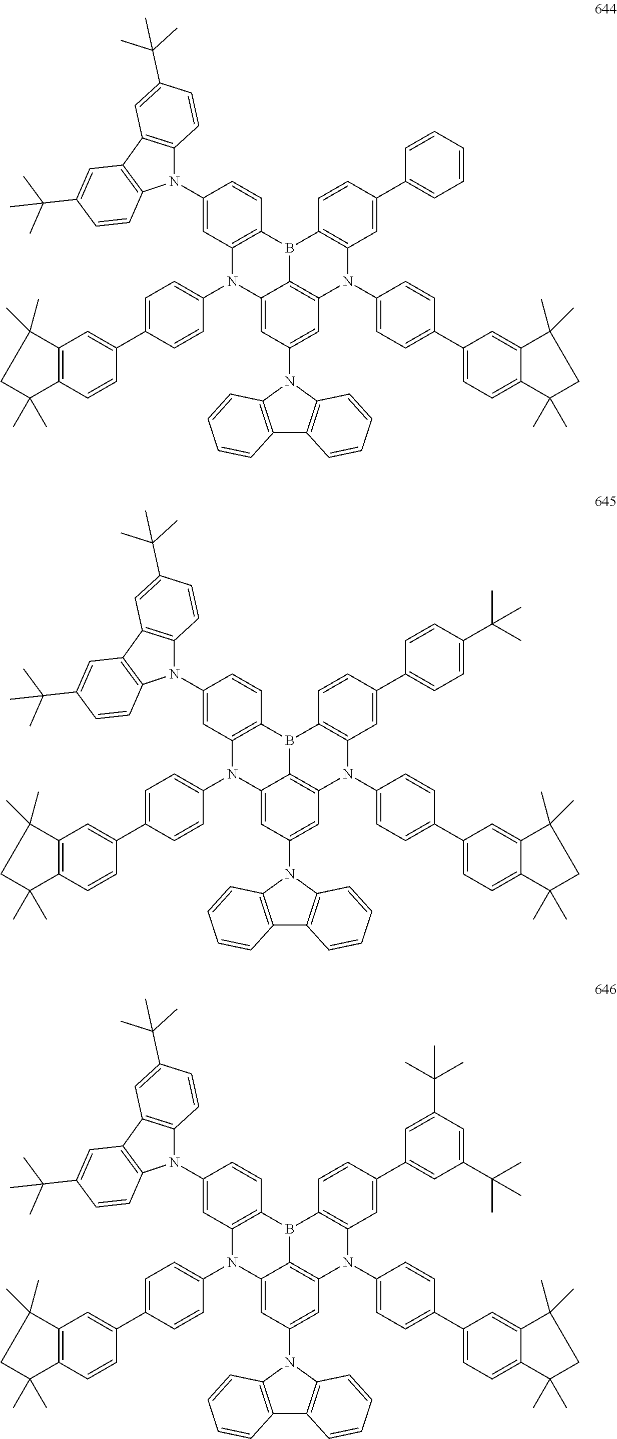

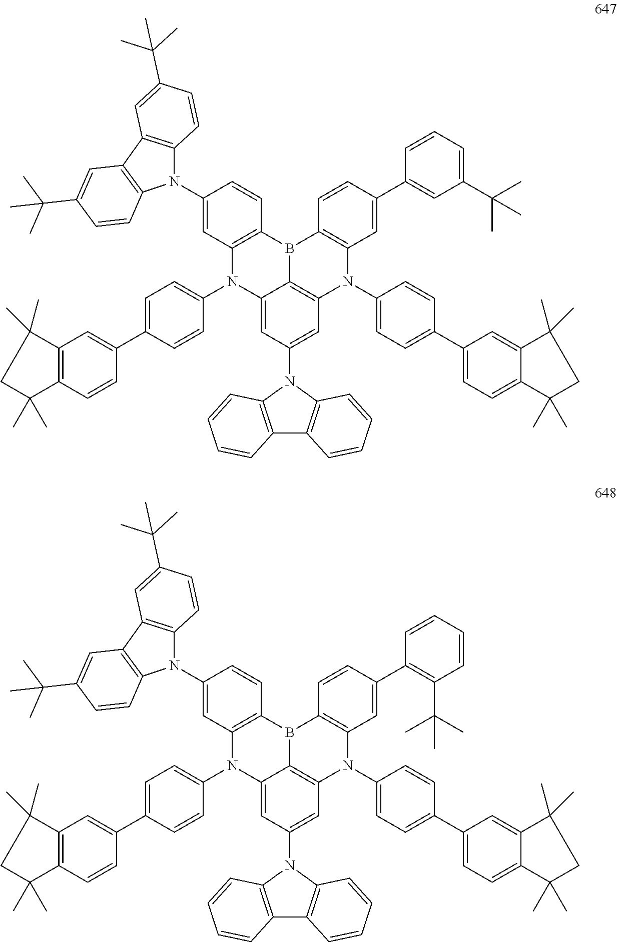 [SMILES: CC(C)(C)c1cccc(-c2ccc3c(c2)N(c2ccc(-c4ccc5c(c4)C(C)(C)CC5(C)C)cc2)c2cc(-n4c5ccccc5c5ccccc54)cc4c2B3c2ccc(-n3c5ccc(C(C)(C)C)cc5c5cc(C(C)(C)C)ccc53)cc2N4c2ccc(-c3ccc4c(c3)C(C)(C)CC4(C)C)cc2)c1 ; CC(C)(C)c1ccc2c(c1)c1cc(C(C)(C)C)ccc1n2-c1ccc2c(c1)N(c1ccc(-c3ccc4c(c3)C(C)(C)CC4(C)C)cc1)c1cc(-n3c4ccccc4c4ccccc43)cc3c1B2c1ccc(-c2ccccc2C(C)(C)C)cc1N3c1ccc(-c2ccc3c(c2)C(C)(C)CC3(C)C)cc1]

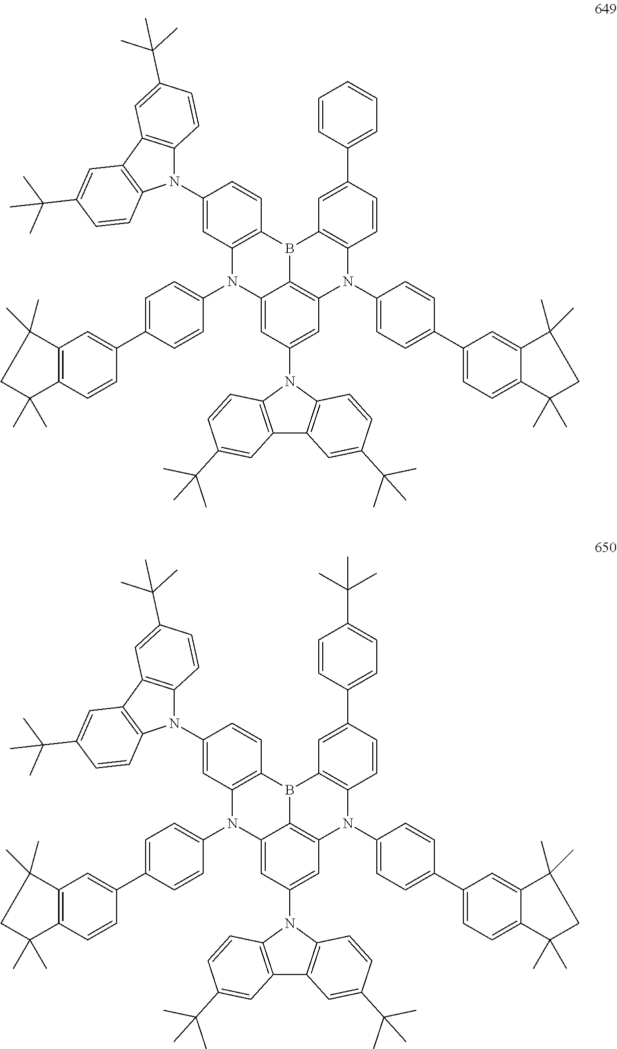 [SMILES: CC(C)(C)c1ccc2c(c1)c1cc(C(C)(C)C)ccc1n2-c1ccc2c(c1)N(c1ccc(-c3ccc4c(c3)C(C)(C)CC4(C)C)cc1)c1cc(-n3c4ccc(C(C)(C)C)cc4c4cc(C(C)(C)C)ccc43)cc3c1B2c1cc(-c2ccccc2)ccc1N3c1ccc(-c2ccc3c(c2)C(C)(C)CC3(C)C)cc1 ; CC(C)(C)c1ccc(-c2ccc3c(c2)B2c4ccc(-n5c6ccc(C(C)(C)C)cc6c6cc(C(C)(C)C)ccc65)cc4N(c4ccc(-c5ccc6c(c5)C(C)(C)CC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CC4(C)C)cc2)cc1]

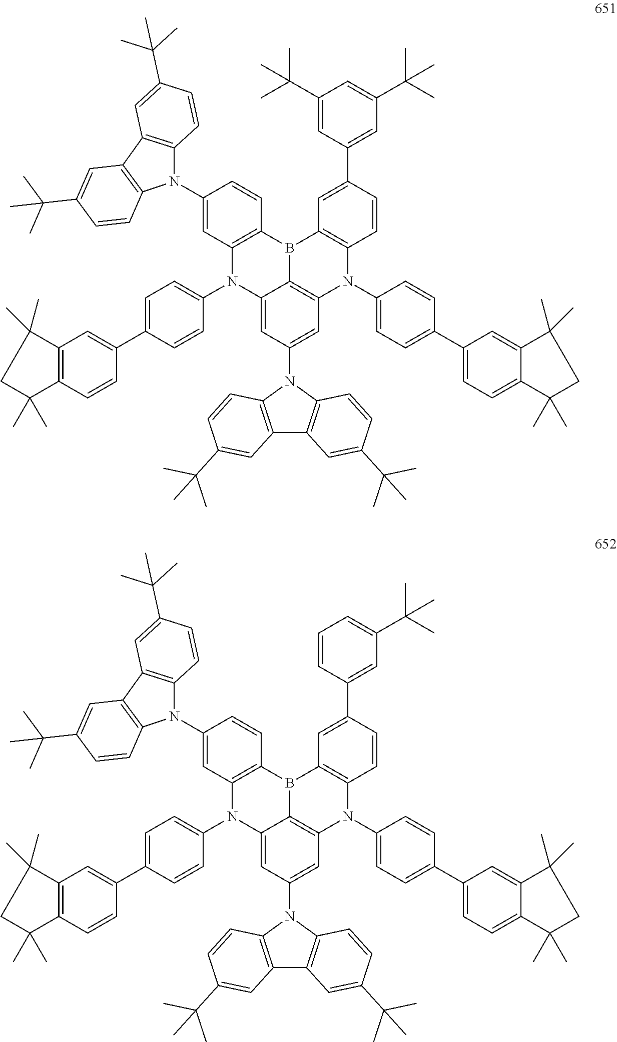 [SMILES: CC(C)(C)c1cc(-c2ccc3c(c2)B2c4ccc(-n5c6ccc(C(C)(C)C)cc6c6cc(C(C)(C)C)ccc65)cc4N(c4ccc(-c5ccc6c(c5)C(C)(C)CC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CC4(C)C)cc2)cc(C(C)(C)C)c1 ; CC(C)(C)c1cccc(-c2ccc3c(c2)B2c4ccc(-n5c6ccc(C(C)(C)C)cc6c6cc(C(C)(C)C)ccc65)cc4N(c4ccc(-c5ccc6c(c5)C(C)(C)CC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CC4(C)C)cc2)c1]

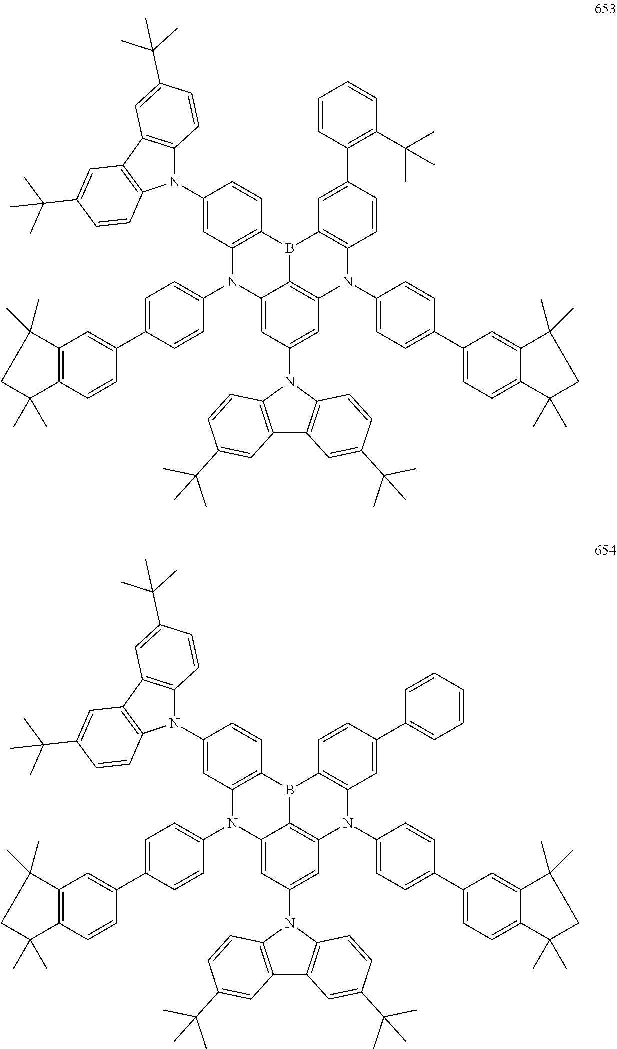 [SMILES: CC(C)(C)c1ccc2c(c1)c1cc(C(C)(C)C)ccc1n2-c1ccc2c(c1)N(c1ccc(-c3ccc4c(c3)C(C)(C)CC4(C)C)cc1)c1cc(-n3c4ccc(C(C)(C)C)cc4c4cc(C(C)(C)C)ccc43)cc3c1B2c1cc(-c2ccccc2C(C)(C)C)ccc1N3c1ccc(-c2ccc3c(c2)C(C)(C)CC3(C)C)cc1 ; CC(C)(C)c1ccc2c(c1)c1cc(C(C)(C)C)ccc1n2-c1ccc2c(c1)N(c1ccc(-c3ccc4c(c3)C(C)(C)CC4(C)C)cc1)c1cc(-n3c4ccc(C(C)(C)C)cc4c4cc(C(C)(C)C)ccc43)cc3c1B2c1ccc(-c2ccccc2)cc1N3c1ccc(-c2ccc3c(c2)C(C)(C)CC3(C)C)cc1]

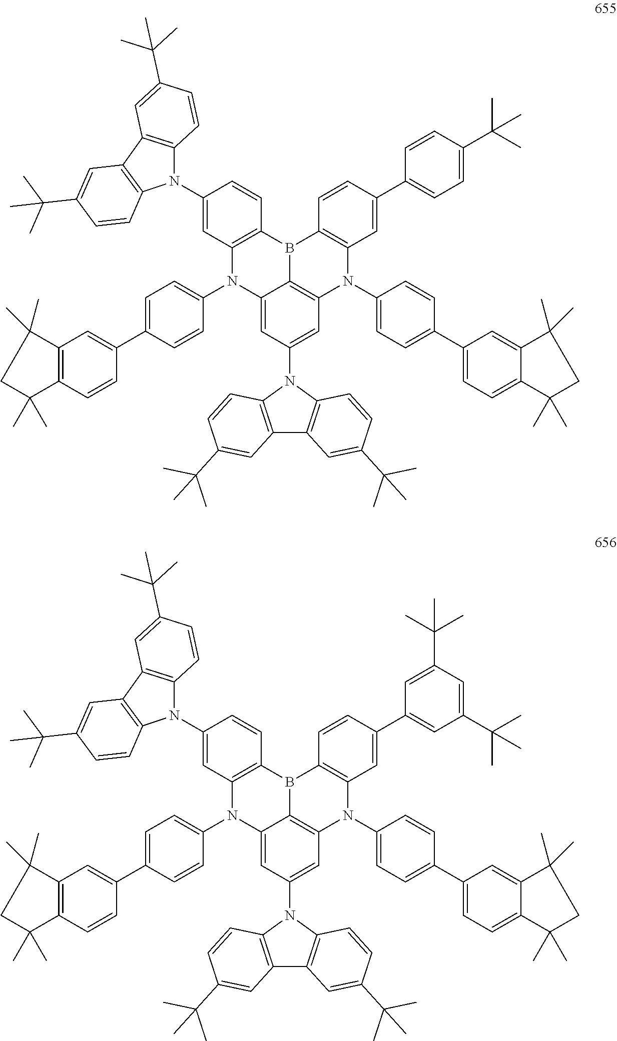 [SMILES: CC(C)(C)c1ccc(-c2ccc3c(c2)N(c2ccc(-c4ccc5c(c4)C(C)(C)CC5(C)C)cc2)c2cc(-n4c5ccc(C(C)(C)C)cc5c5cc(C(C)(C)C)ccc54)cc4c2B3c2ccc(-n3c5ccc(C(C)(C)C)cc5c5cc(C(C)(C)C)ccc53)cc2N4c2ccc(-c3ccc4c(c3)C(C)(C)CC4(C)C)cc2)cc1 ; CC(C)(C)c1cc(-c2ccc3c(c2)N(c2ccc(-c4ccc5c(c4)C(C)(C)CC5(C)C)cc2)c2cc(-n4c5ccc(C(C)(C)C)cc5c5cc(C(C)(C)C)ccc54)cc4c2B3c2ccc(-n3c5ccc(C(C)(C)C)cc5c5cc(C(C)(C)C)ccc53)cc2N4c2ccc(-c3ccc4c(c3)C(C)(C)CC4(C)C)cc2)cc(C(C)(C)C)c1]

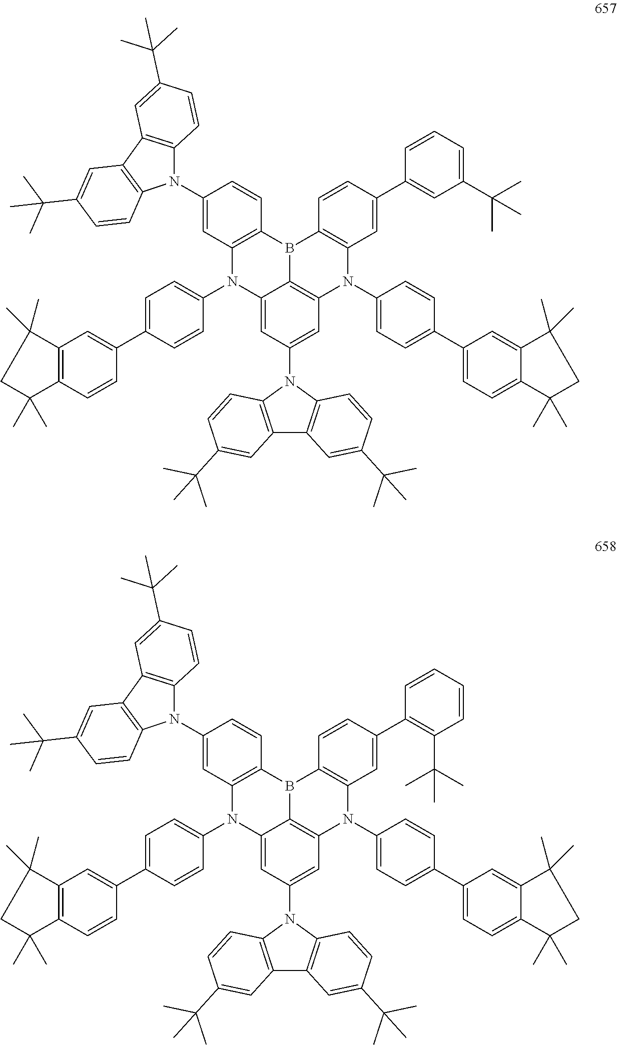 [SMILES: CC(C)(C)c1cccc(-c2ccc3c(c2)N(c2ccc(-c4ccc5c(c4)C(C)(C)CC5(C)C)cc2)c2cc(-n4c5ccc(C(C)(C)C)cc5c5cc(C(C)(C)C)ccc54)cc4c2B3c2ccc(-n3c5ccc(C(C)(C)C)cc5c5cc(C(C)(C)C)ccc53)cc2N4c2ccc(-c3ccc4c(c3)C(C)(C)CC4(C)C)cc2)c1 ; CC(C)(C)c1ccc2c(c1)c1cc(C(C)(C)C)ccc1n2-c1ccc2c(c1)N(c1ccc(-c3ccc4c(c3)C(C)(C)CC4(C)C)cc1)c1cc(-n3c4ccc(C(C)(C)C)cc4c4cc(C(C)(C)C)ccc43)cc3c1B2c1ccc(-c2ccccc2C(C)(C)C)cc1N3c1ccc(-c2ccc3c(c2)C(C)(C)CC3(C)C)cc1]

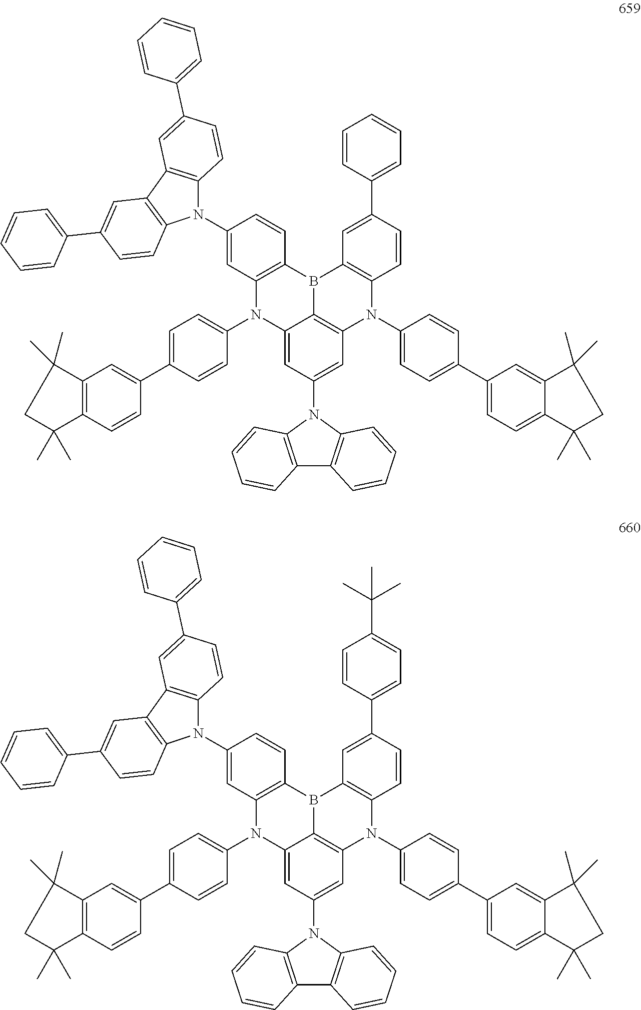 [SMILES: CC1(C)CC(C)(C)c2cc(-c3ccc(N4c5ccc(-c6ccccc6)cc5B5c6ccc(-n7c8ccc(-c9ccccc9)cc8c8cc(-c9ccccc9)ccc87)cc6N(c6ccc(-c7ccc8c(c7)C(C)(C)CC8(C)C)cc6)c6cc(-n7c8ccccc8c8ccccc87)cc4c65)cc3)ccc21 ; CC(C)(C)c1ccc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CC6(C)C)cc4)c4cc(-n5c6ccccc6c6ccccc65)cc(c42)N3c2ccc(-c3ccc4c(c3)C(C)(C)CC4(C)C)cc2)cc1]

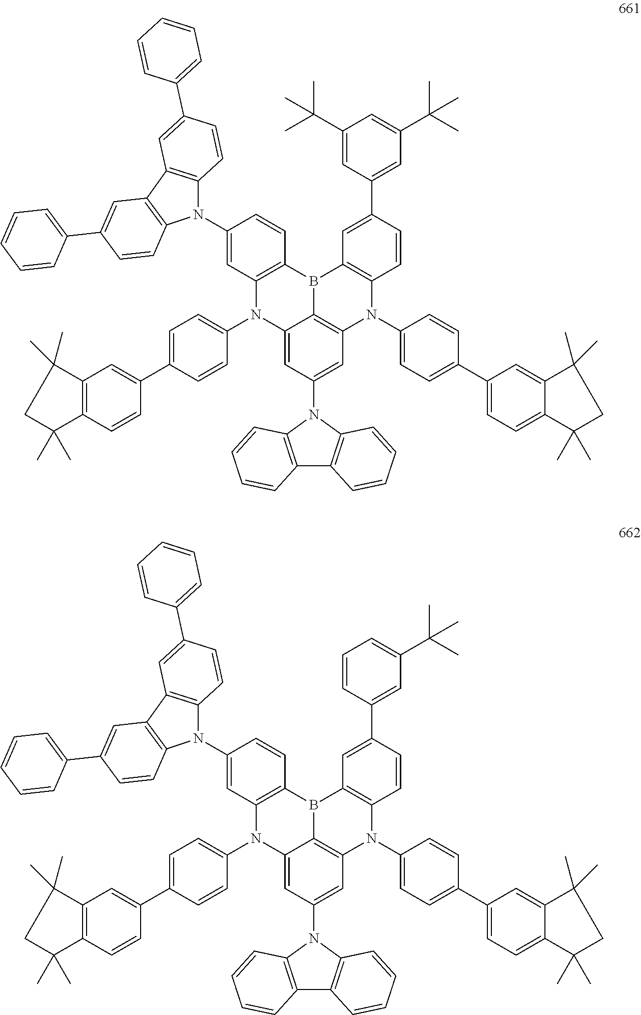 [SMILES: CC(C)(C)c1cc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CC6(C)C)cc4)c4cc(-n5c6ccccc6c6ccccc65)cc(c42)N3c2ccc(-c3ccc4c(c3)C(C)(C)CC4(C)C)cc2)cc(C(C)(C)C)c1 ; CC(C)(C)c1cccc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CC6(C)C)cc4)c4cc(-n5c6ccccc6c6ccccc65)cc(c42)N3c2ccc(-c3ccc4c(c3)C(C)(C)CC4(C)C)cc2)c1]

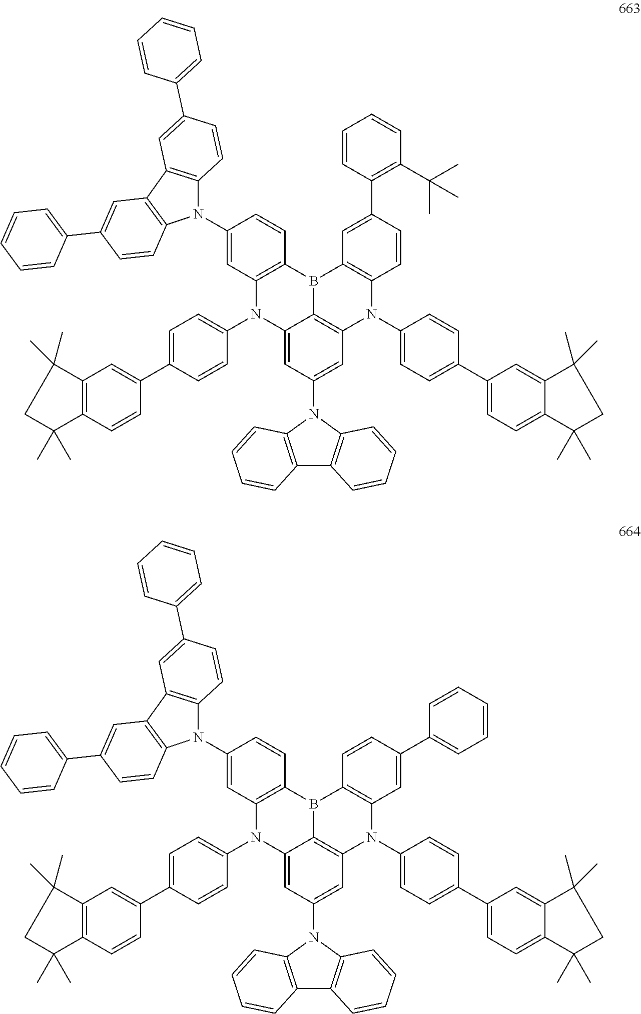 [SMILES: CC(C)(C)c1ccccc1-c1ccc2c(c1)B1c3ccc(-n4c5ccc(-c6ccccc6)cc5c5cc(-c6ccccc6)ccc54)cc3N(c3ccc(-c4ccc5c(c4)C(C)(C)CC5(C)C)cc3)c3cc(-n4c5ccccc5c5ccccc54)cc(c31)N2c1ccc(-c2ccc3c(c2)C(C)(C)CC3(C)C)cc1 ; CC1(C)CC(C)(C)c2cc(-c3ccc(N4c5cc(-c6ccccc6)ccc5B5c6ccc(-n7c8ccc(-c9ccccc9)cc8c8cc(-c9ccccc9)ccc87)cc6N(c6ccc(-c7ccc8c(c7)C(C)(C)CC8(C)C)cc6)c6cc(-n7c8ccccc8c8ccccc87)cc4c65)cc3)ccc21]

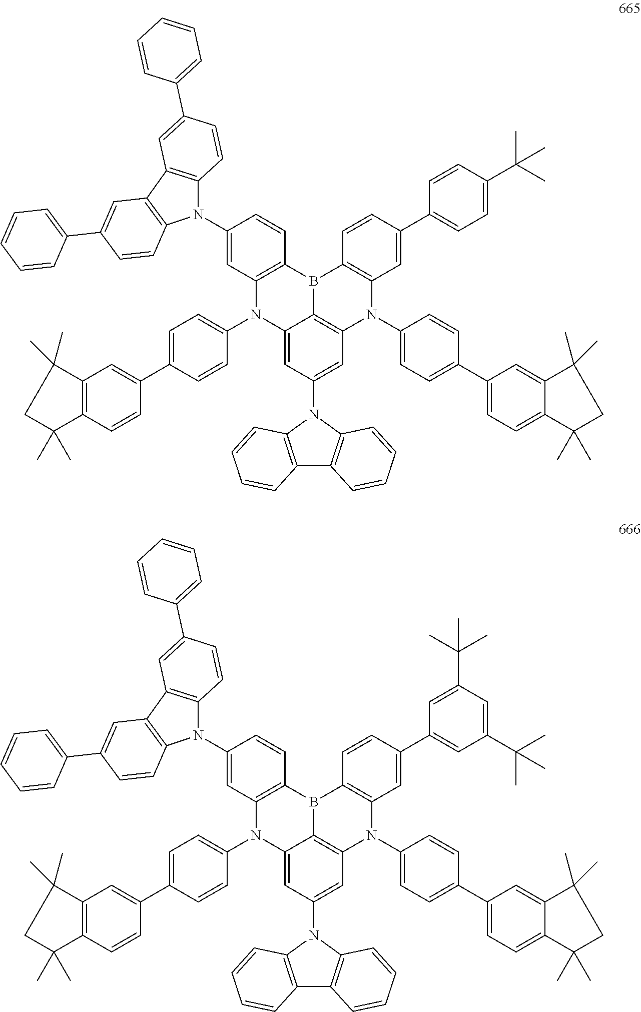 [SMILES: CC(C)(C)c1ccc(-c2ccc3c(c2)N(c2ccc(-c4ccc5c(c4)C(C)(C)CC5(C)C)cc2)c2cc(-n4c5ccccc5c5ccccc54)cc4c2B3c2ccc(-n3c5ccc(-c6ccccc6)cc5c5cc(-c6ccccc6)ccc53)cc2N4c2ccc(-c3ccc4c(c3)C(C)(C)CC4(C)C)cc2)cc1 ; CC(C)(C)c1cc(-c2ccc3c(c2)N(c2ccc(-c4ccc5c(c4)C(C)(C)CC5(C)C)cc2)c2cc(-n4c5ccccc5c5ccccc54)cc4c2B3c2ccc(-n3c5ccc(-c6ccccc6)cc5c5cc(-c6ccccc6)ccc53)cc2N4c2ccc(-c3ccc4c(c3)C(C)(C)CC4(C)C)cc2)cc(C(C)(C)C)c1]

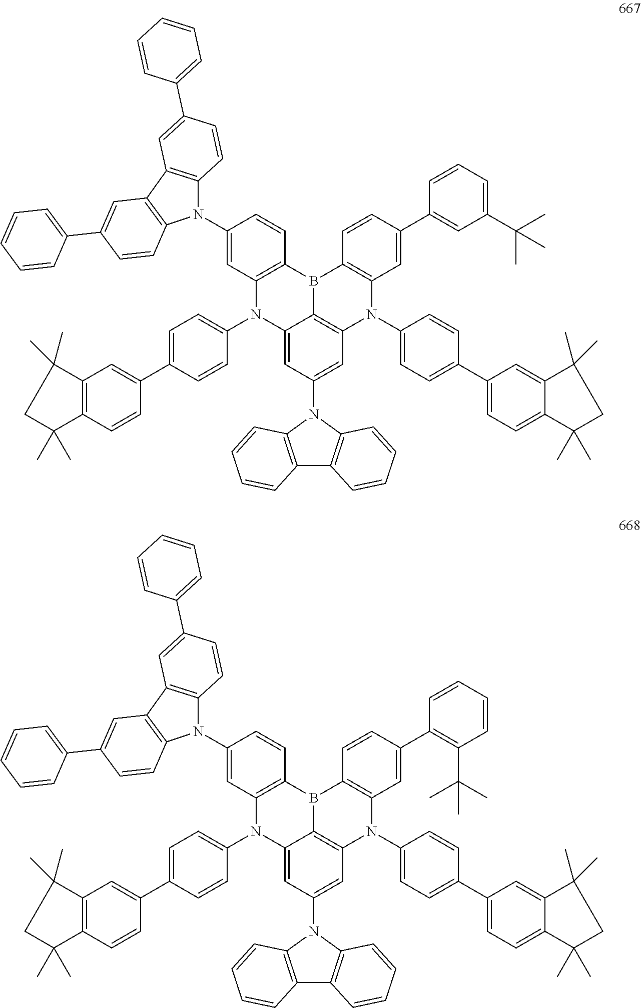 [SMILES: CC(C)(C)c1cccc(-c2ccc3c(c2)N(c2ccc(-c4ccc5c(c4)C(C)(C)CC5(C)C)cc2)c2cc(-n4c5ccccc5c5ccccc54)cc4c2B3c2ccc(-n3c5ccc(-c6ccccc6)cc5c5cc(-c6ccccc6)ccc53)cc2N4c2ccc(-c3ccc4c(c3)C(C)(C)CC4(C)C)cc2)c1 ; CC(C)(C)c1ccccc1-c1ccc2c(c1)N(c1ccc(-c3ccc4c(c3)C(C)(C)CC4(C)C)cc1)c1cc(-n3c4ccccc4c4ccccc43)cc3c1B2c1ccc(-n2c4ccc(-c5ccccc5)cc4c4cc(-c5ccccc5)ccc42)cc1N3c1ccc(-c2ccc3c(c2)C(C)(C)CC3(C)C)cc1]

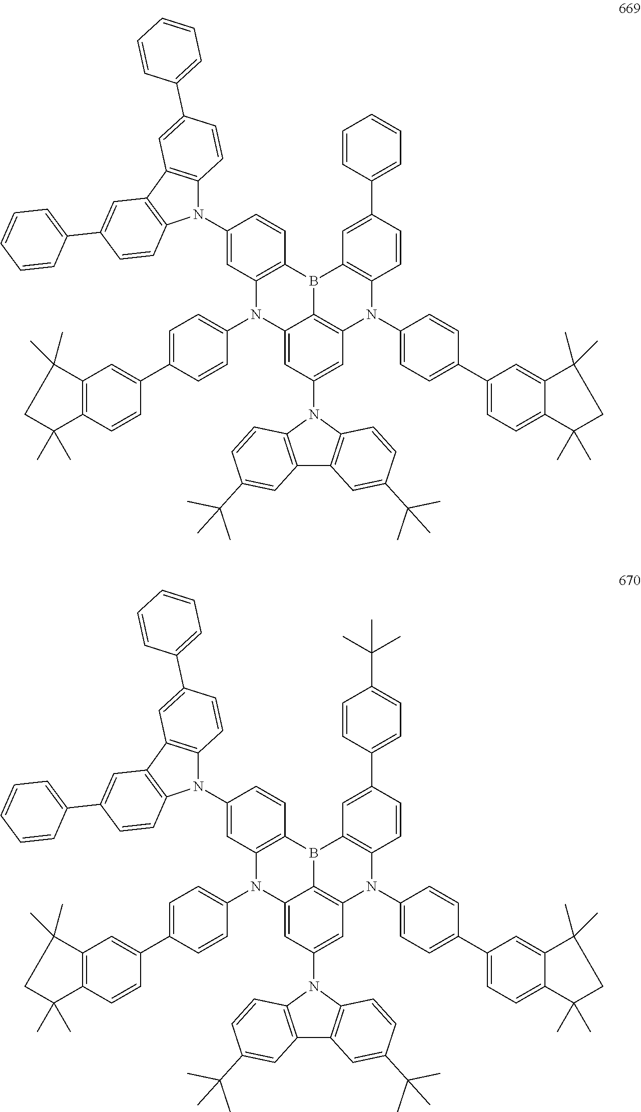 [SMILES: CC(C)(C)c1ccc2c(c1)c1cc(C(C)(C)C)ccc1n2-c1cc2c3c(c1)N(c1ccc(-c4ccc5c(c4)C(C)(C)CC5(C)C)cc1)c1cc(-n4c5ccc(-c6ccccc6)cc5c5cc(-c6ccccc6)ccc54)ccc1B3c1cc(-c3ccccc3)ccc1N2c1ccc(-c2ccc3c(c2)C(C)(C)CC3(C)C)cc1 ; CC(C)(C)c1ccc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CC4(C)C)cc2)cc1]

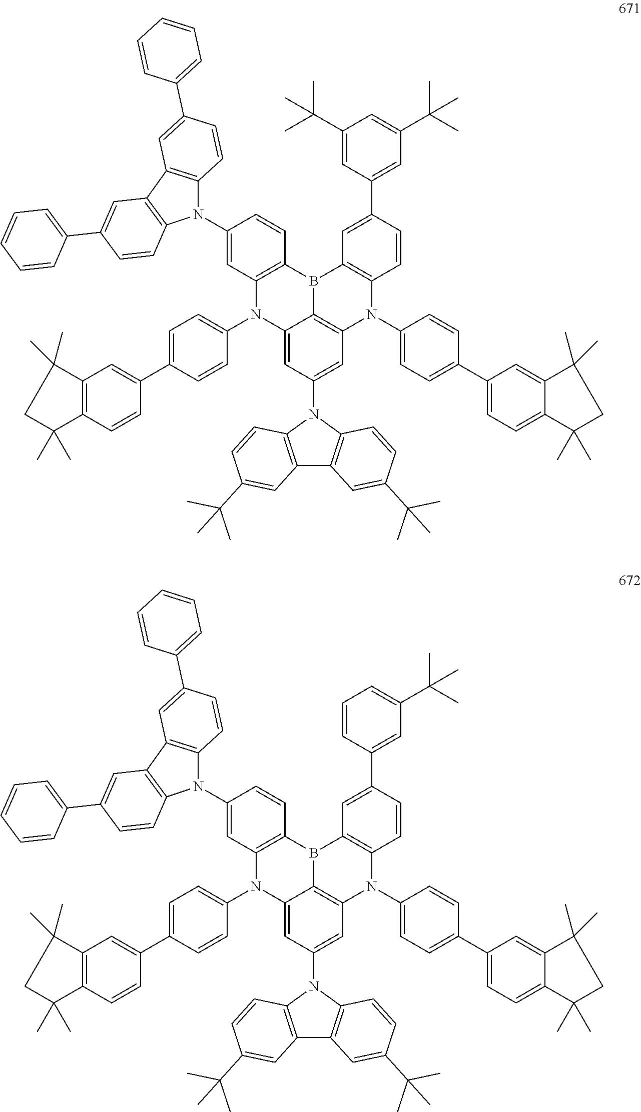 [SMILES: CC(C)(C)c1cc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CC4(C)C)cc2)cc(C(C)(C)C)c1 ; CC(C)(C)c1cccc(-c2ccc3c(c2)B2c4ccc(-n5c6ccc(-c7ccccc7)cc6c6cc(-c7ccccc7)ccc65)cc4N(c4ccc(-c5ccc6c(c5)C(C)(C)CC6(C)C)cc4)c4cc(-n5c6ccc(C(C)(C)C)cc6c6cc(C(C)(C)C)ccc65)cc(c42)N3c2ccc(-c3ccc4c(c3)C(C)(C)CC4(C)C)cc2)c1]

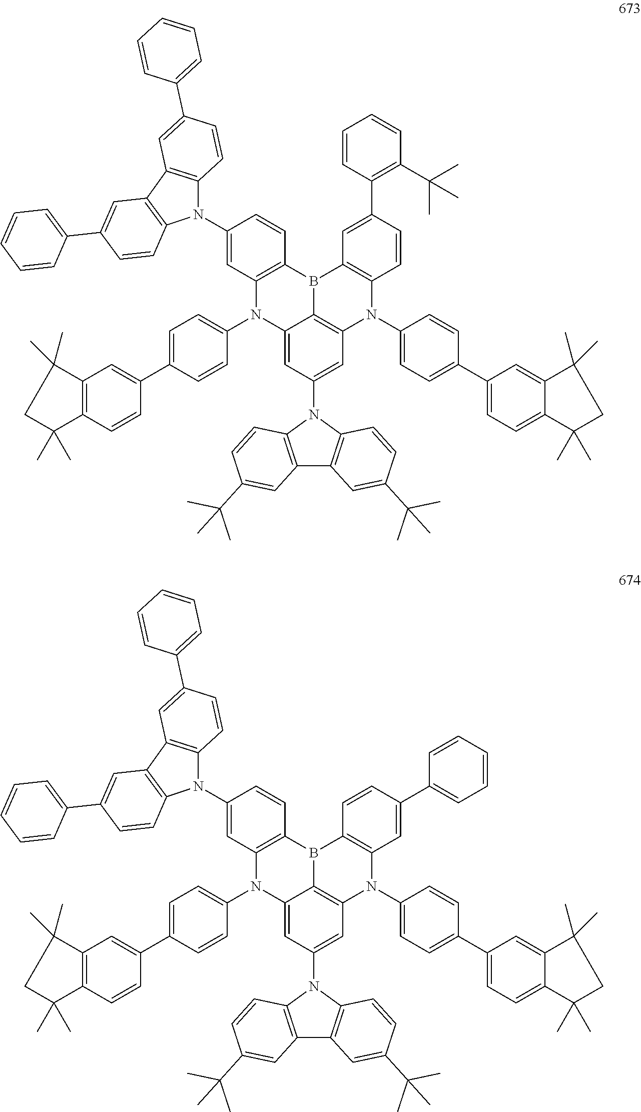 [SMILES: CC(C)(C)c1ccc2c(c1)c1cc(C(C)(C)C)ccc1n2-c1cc2c3c(c1)N(c1ccc(-c4ccc5c(c4)C(C)(C)CC5(C)C)cc1)c1cc(-n4c5ccc(-c6ccccc6)cc5c5cc(-c6ccccc6)ccc54)ccc1B3c1cc(-c3ccccc3C(C)(C)C)ccc1N2c1ccc(-c2ccc3c(c2)C(C)(C)CC3(C)C)cc1 ; CC(C)(C)c1ccc2c(c1)c1cc(C(C)(C)C)ccc1n2-c1cc2c3c(c1)N(c1ccc(-c4ccc5c(c4)C(C)(C)CC5(C)C)cc1)c1cc(-n4c5ccc(-c6ccccc6)cc5c5cc(-c6ccccc6)ccc54)ccc1B3c1ccc(-c3ccccc3)cc1N2c1ccc(-c2ccc3c(c2)C(C)(C)CC3(C)C)cc1]

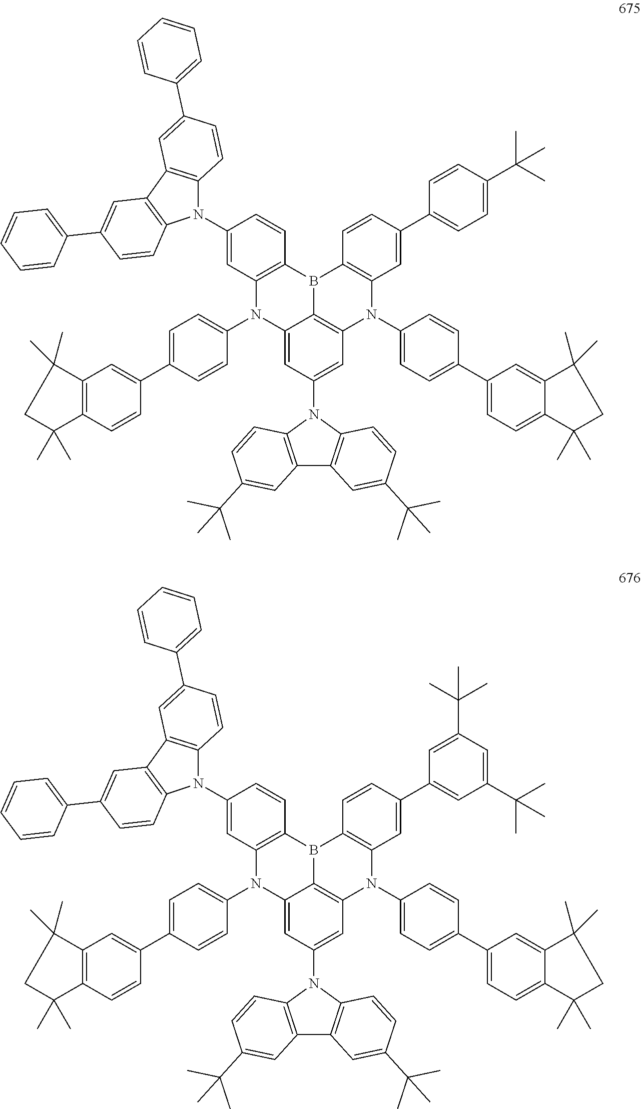 [SMILES: CC(C)(C)c1ccc(-c2ccc3c(c2)N(c2ccc(-c4ccc5c(c4)C(C)(C)CC5(C)C)cc2)c2cc(-n4c5ccc(C(C)(C)C)cc5c5cc(C(C)(C)C)ccc54)cc4c2B3c2ccc(-n3c5ccc(-c6ccccc6)cc5c5cc(-c6ccccc6)ccc53)cc2N4c2ccc(-c3ccc4c(c3)C(C)(C)CC4(C)C)cc2)cc1 ; CC(C)(C)c1cc(-c2ccc3c(c2)N(c2ccc(-c4ccc5c(c4)C(C)(C)CC5(C)C)cc2)c2cc(-n4c5ccc(C(C)(C)C)cc5c5cc(C(C)(C)C)ccc54)cc4c2B3c2ccc(-n3c5ccc(-c6ccccc6)cc5c5cc(-c6ccccc6)ccc53)cc2N4c2ccc(-c3ccc4c(c3)C(C)(C)CC4(C)C)cc2)cc(C(C)(C)C)c1]

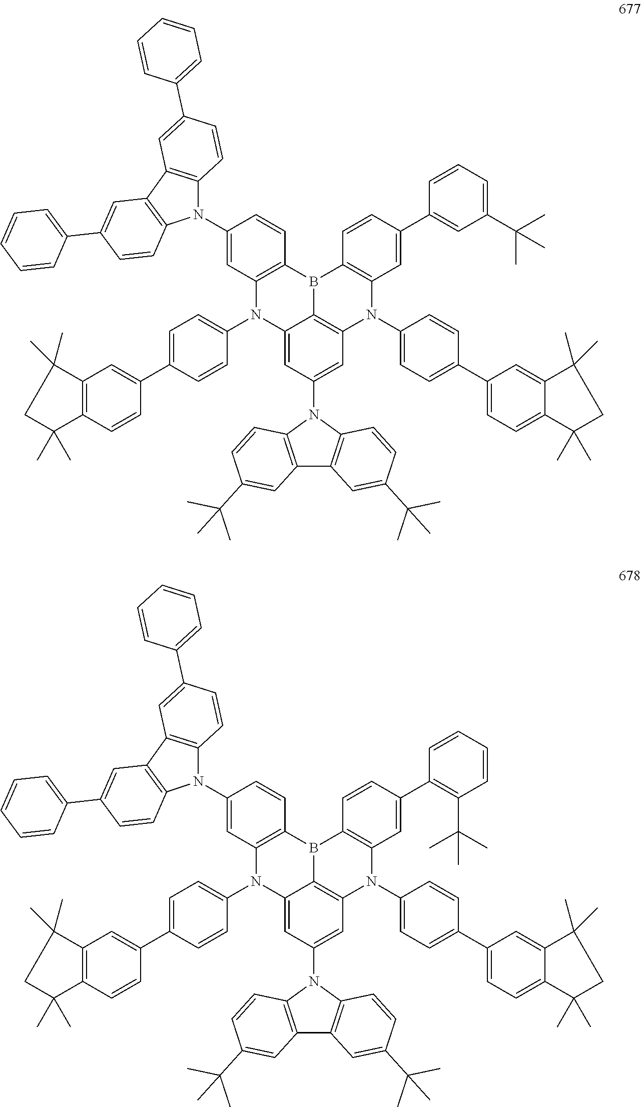 [SMILES: CC(C)(C)c1cccc(-c2ccc3c(c2)N(c2ccc(-c4ccc5c(c4)C(C)(C)CC5(C)C)cc2)c2cc(-n4c5ccc(C(C)(C)C)cc5c5cc(C(C)(C)C)ccc54)cc4c2B3c2ccc(-n3c5ccc(-c6ccccc6)cc5c5cc(-c6ccccc6)ccc53)cc2N4c2ccc(-c3ccc4c(c3)C(C)(C)CC4(C)C)cc2)c1 ; CC(C)(C)c1ccc2c(c1)c1cc(C(C)(C)C)ccc1n2-c1cc2c3c(c1)N(c1ccc(-c4ccc5c(c4)C(C)(C)CC5(C)C)cc1)c1cc(-n4c5ccc(-c6ccccc6)cc5c5cc(-c6ccccc6)ccc54)ccc1B3c1ccc(-c3ccccc3C(C)(C)C)cc1N2c1ccc(-c2ccc3c(c2)C(C)(C)CC3(C)C)cc1]

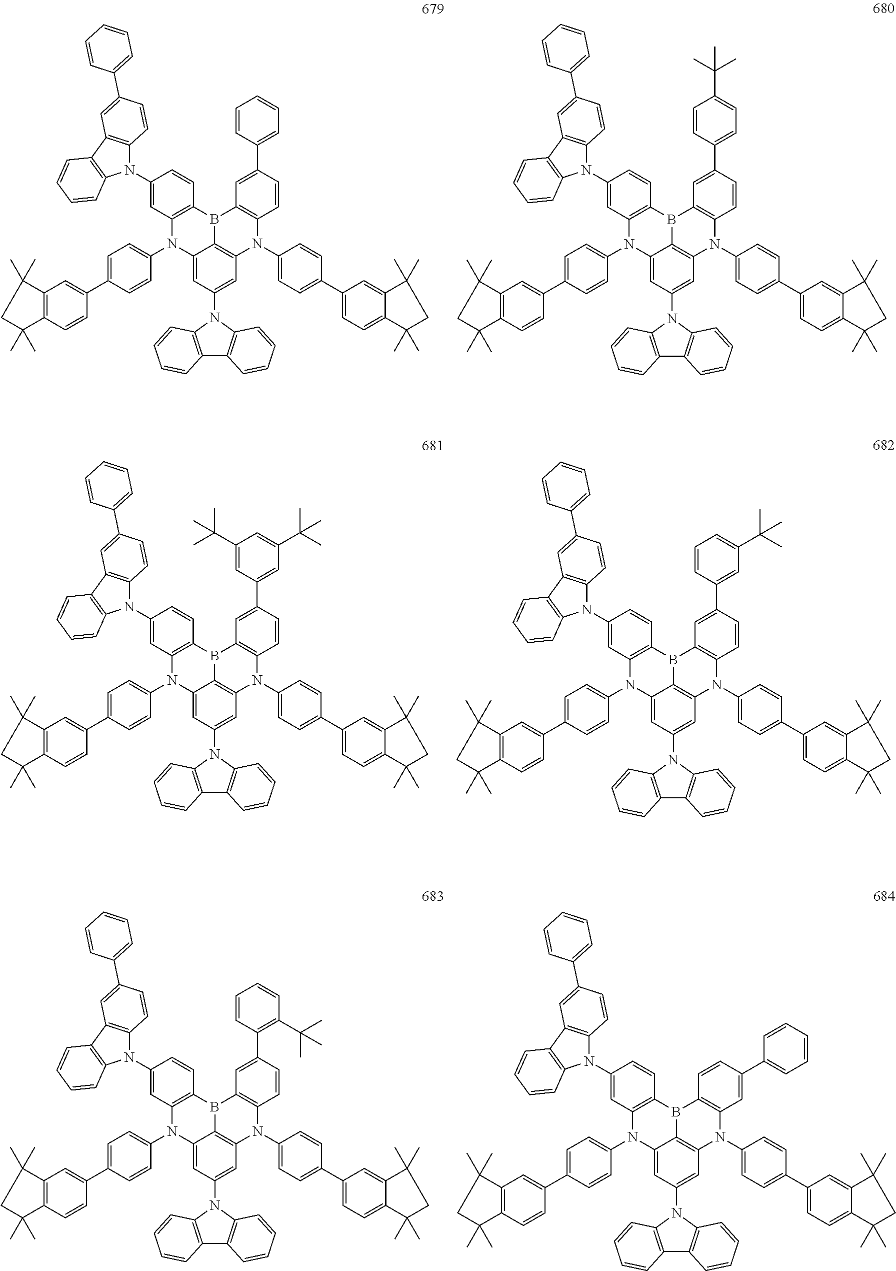

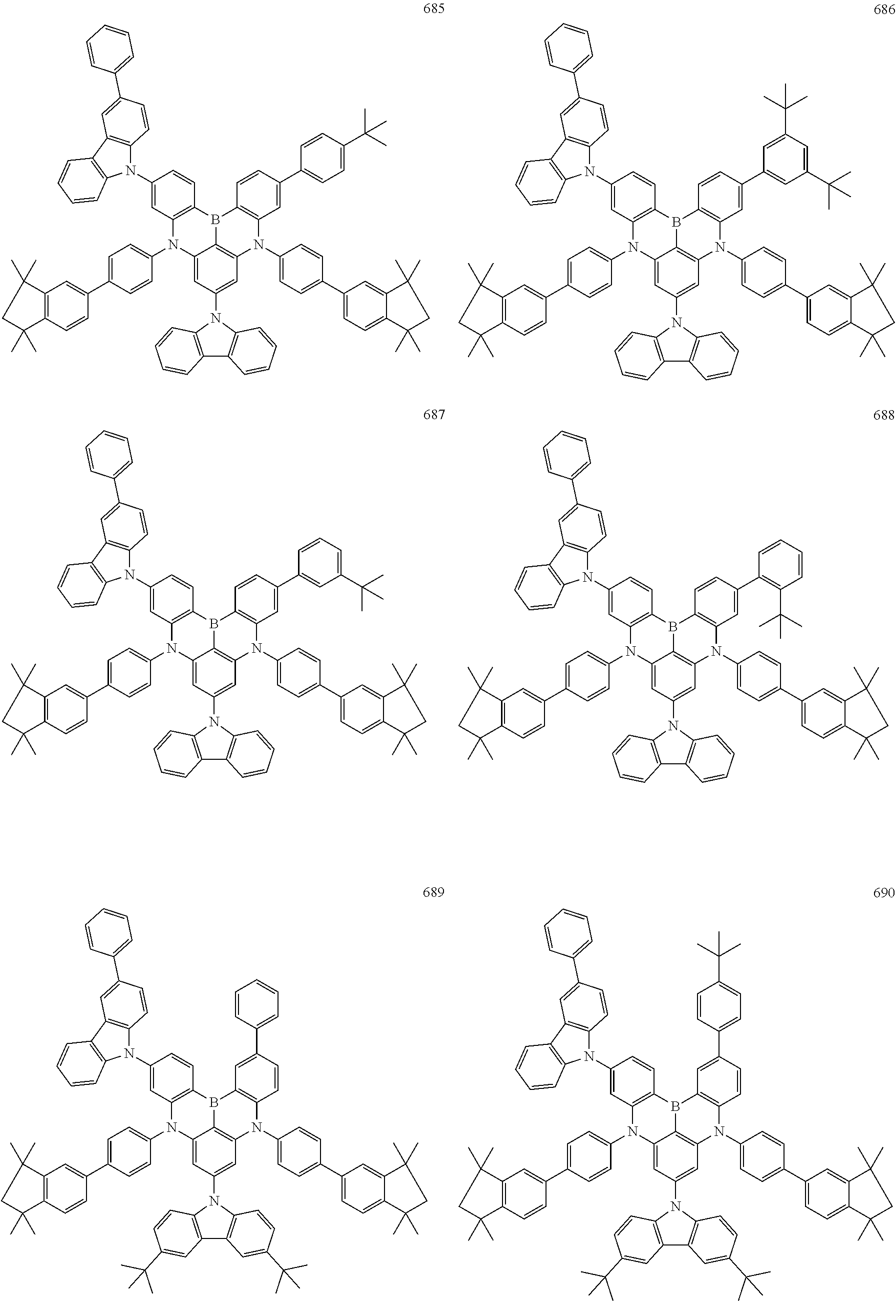

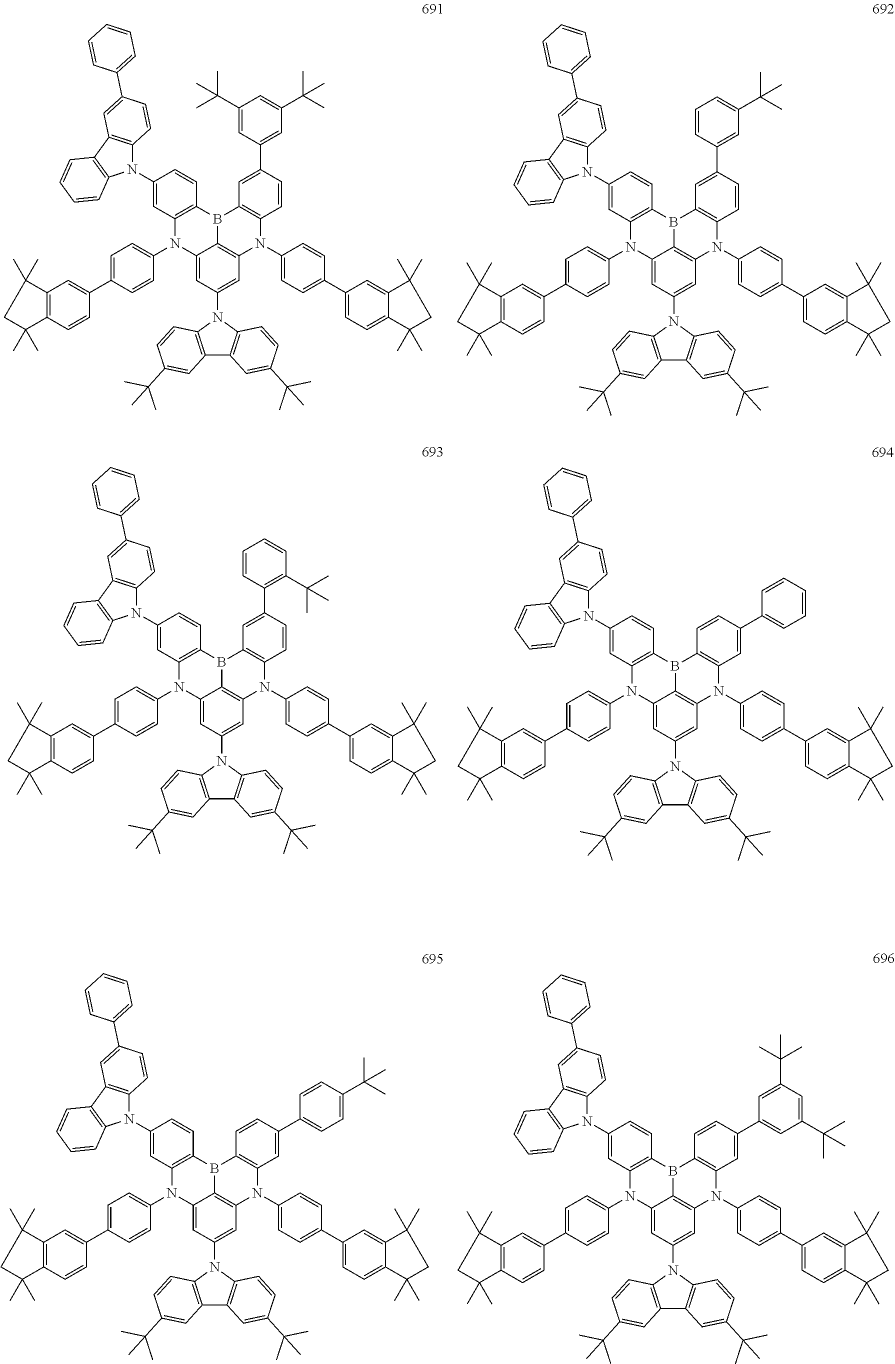

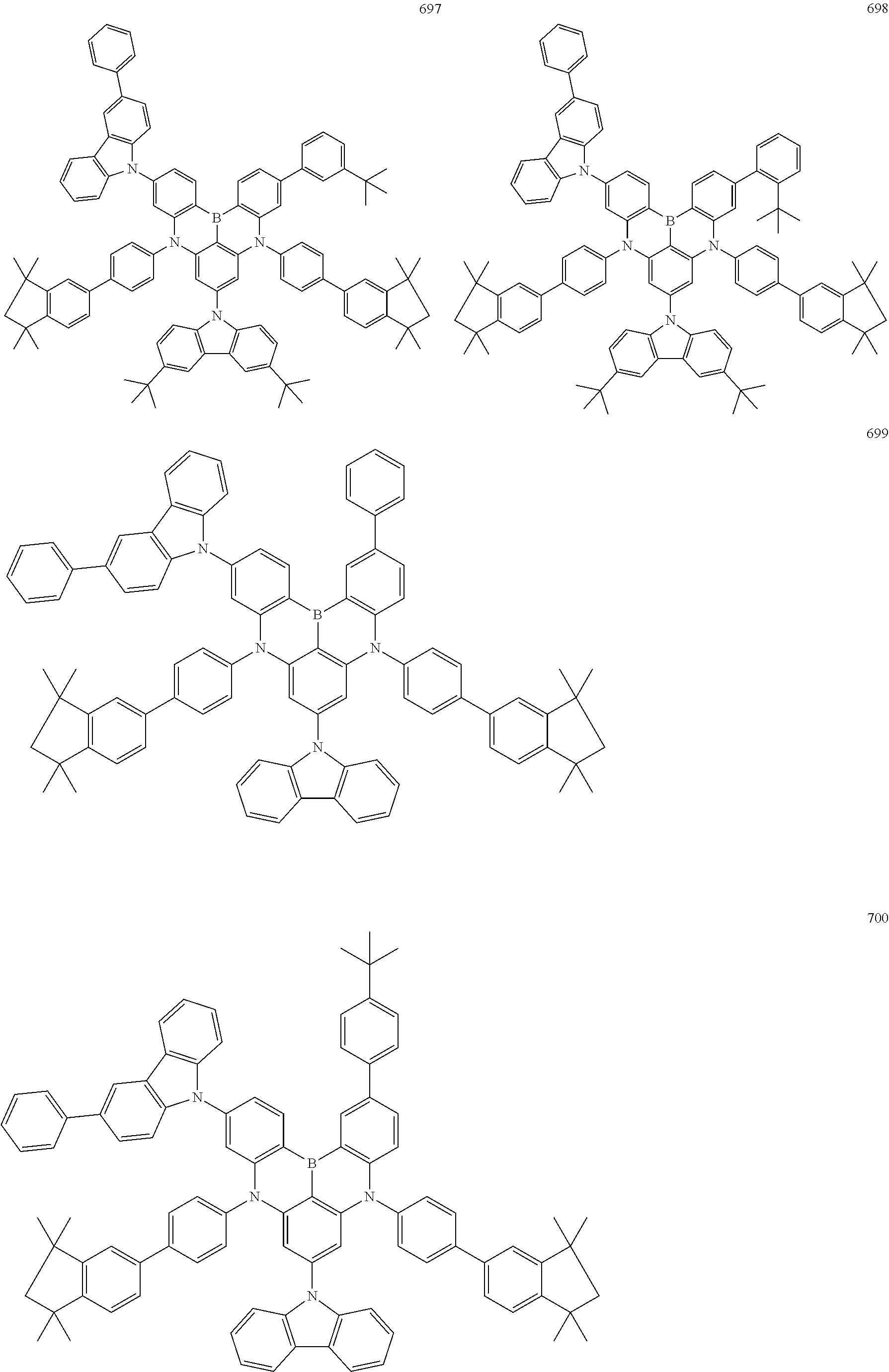

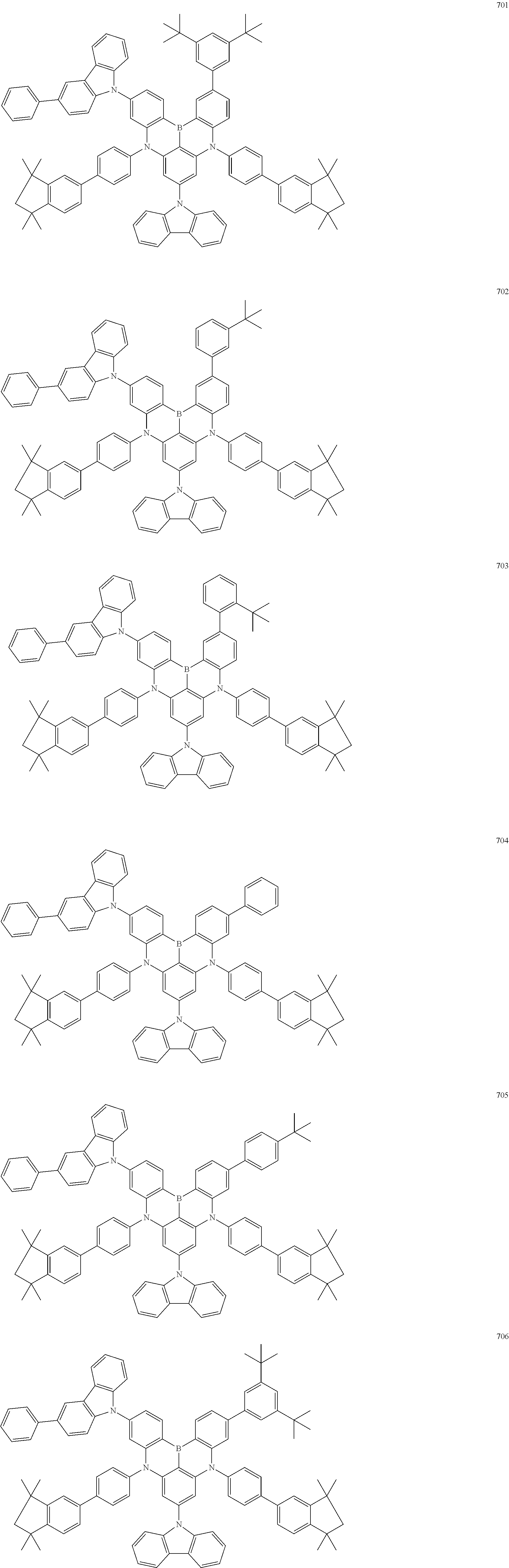

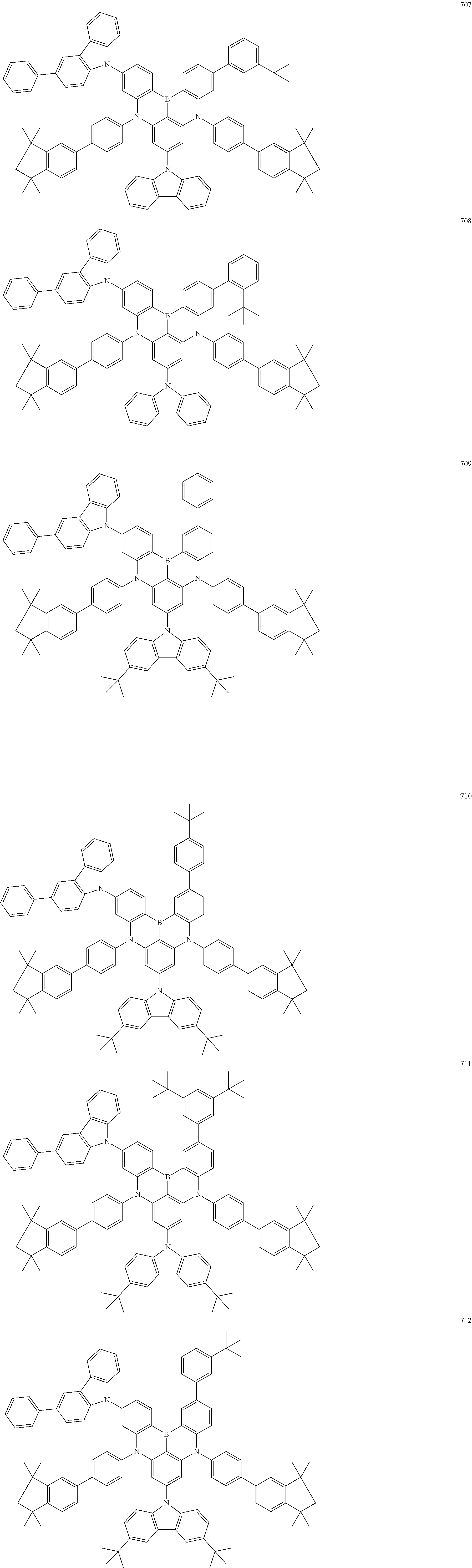

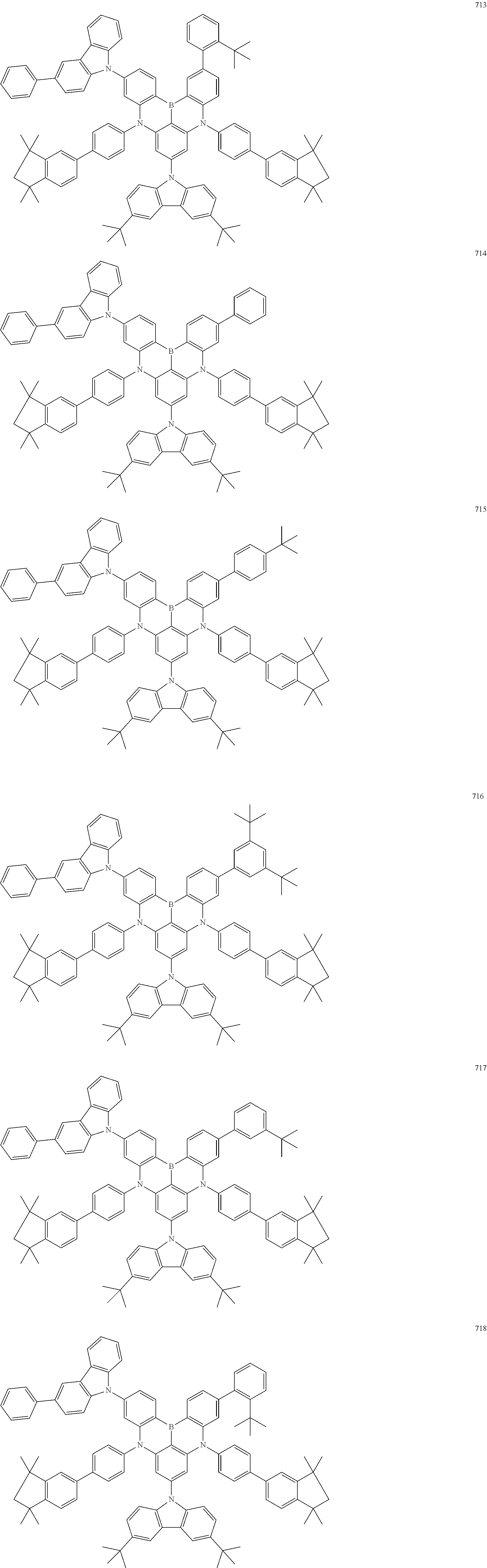

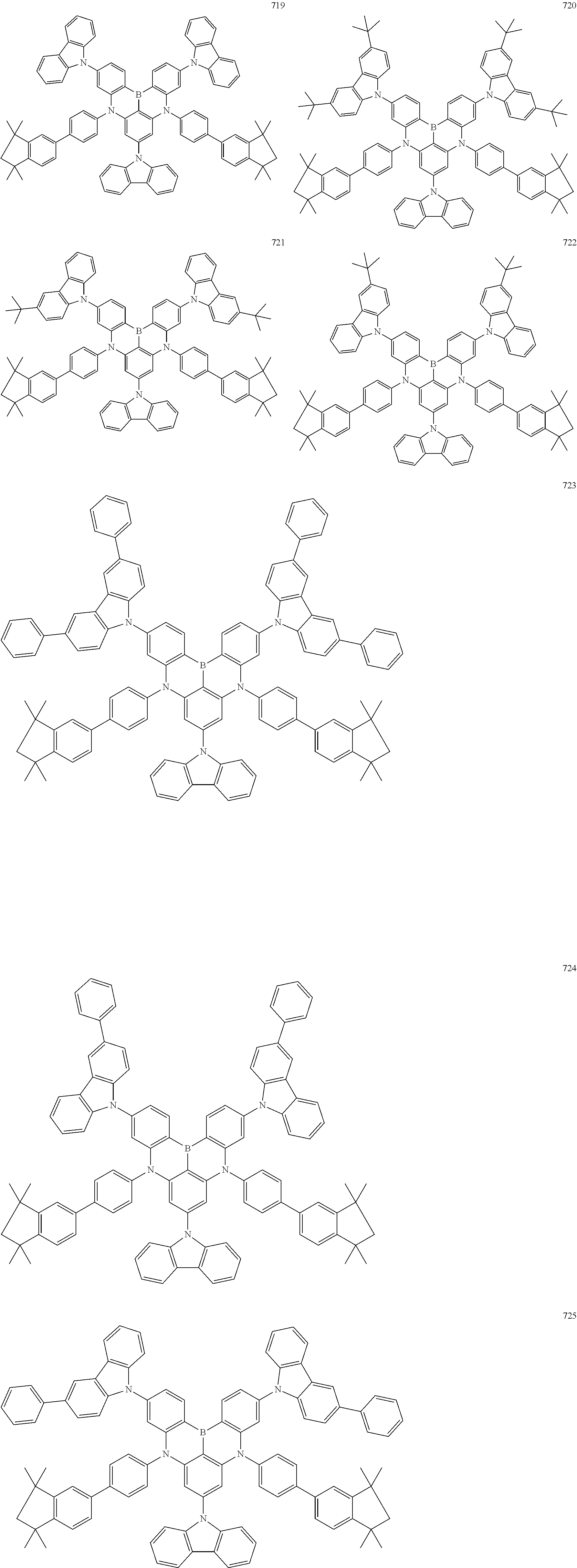

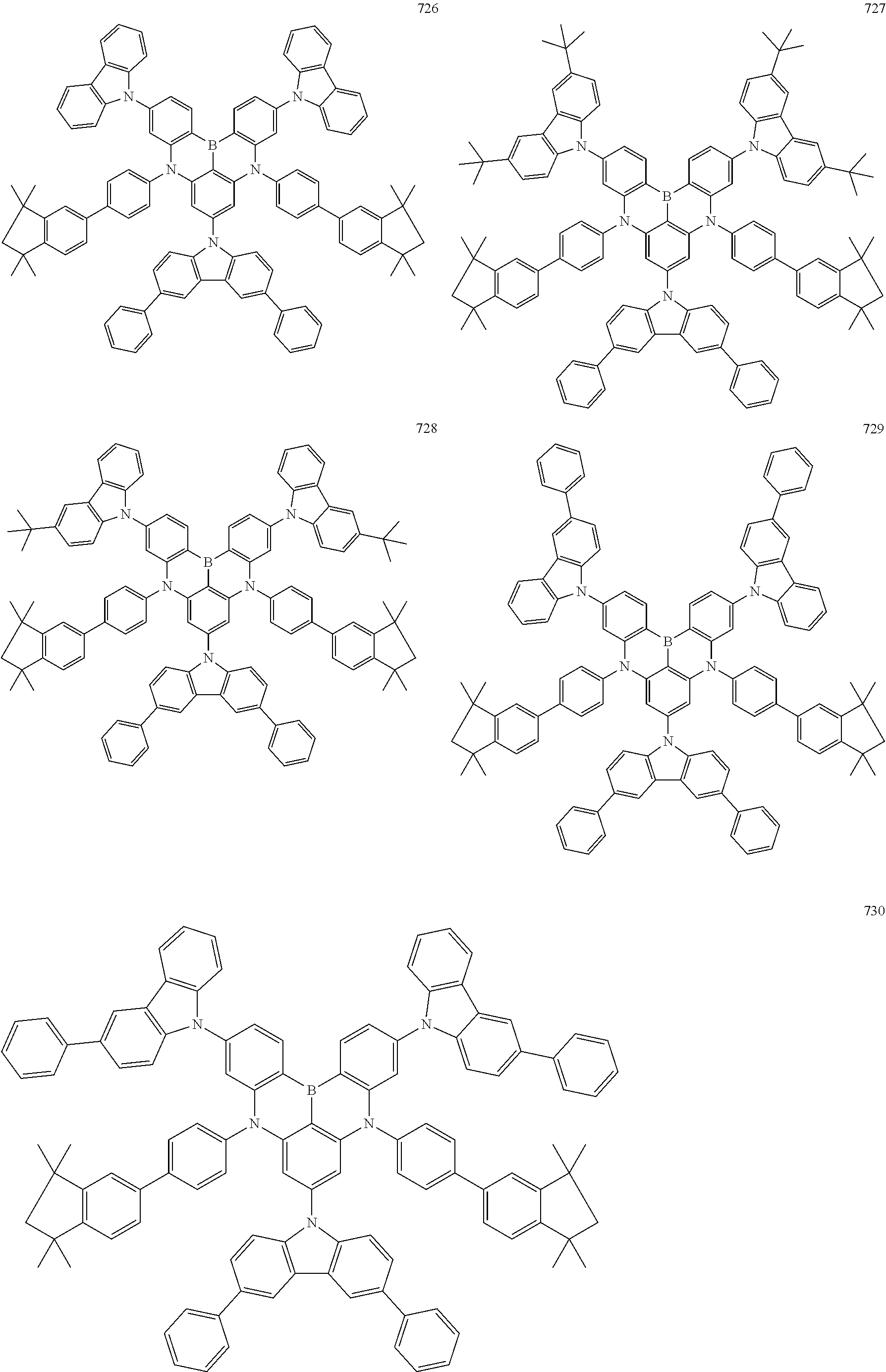

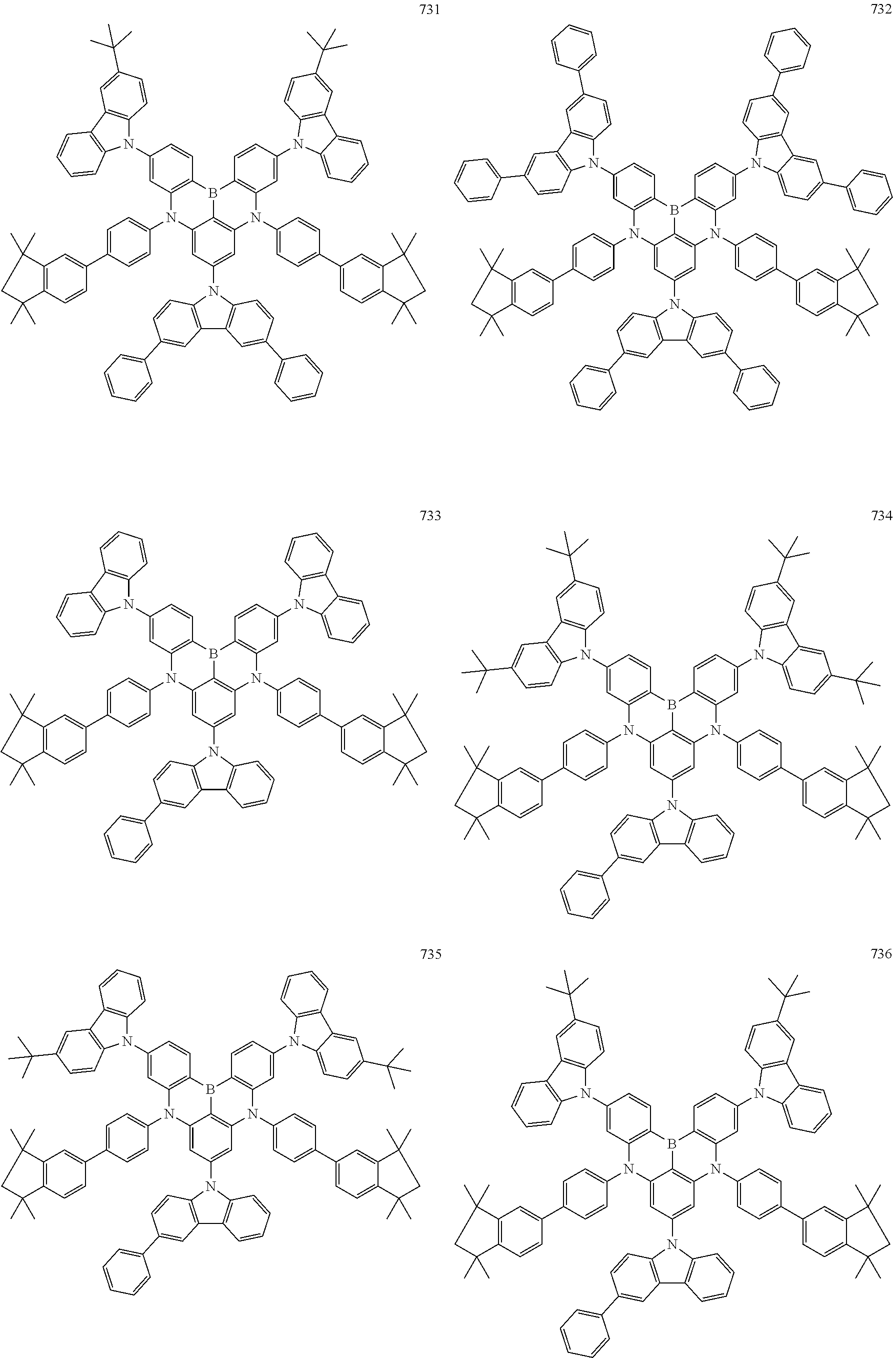

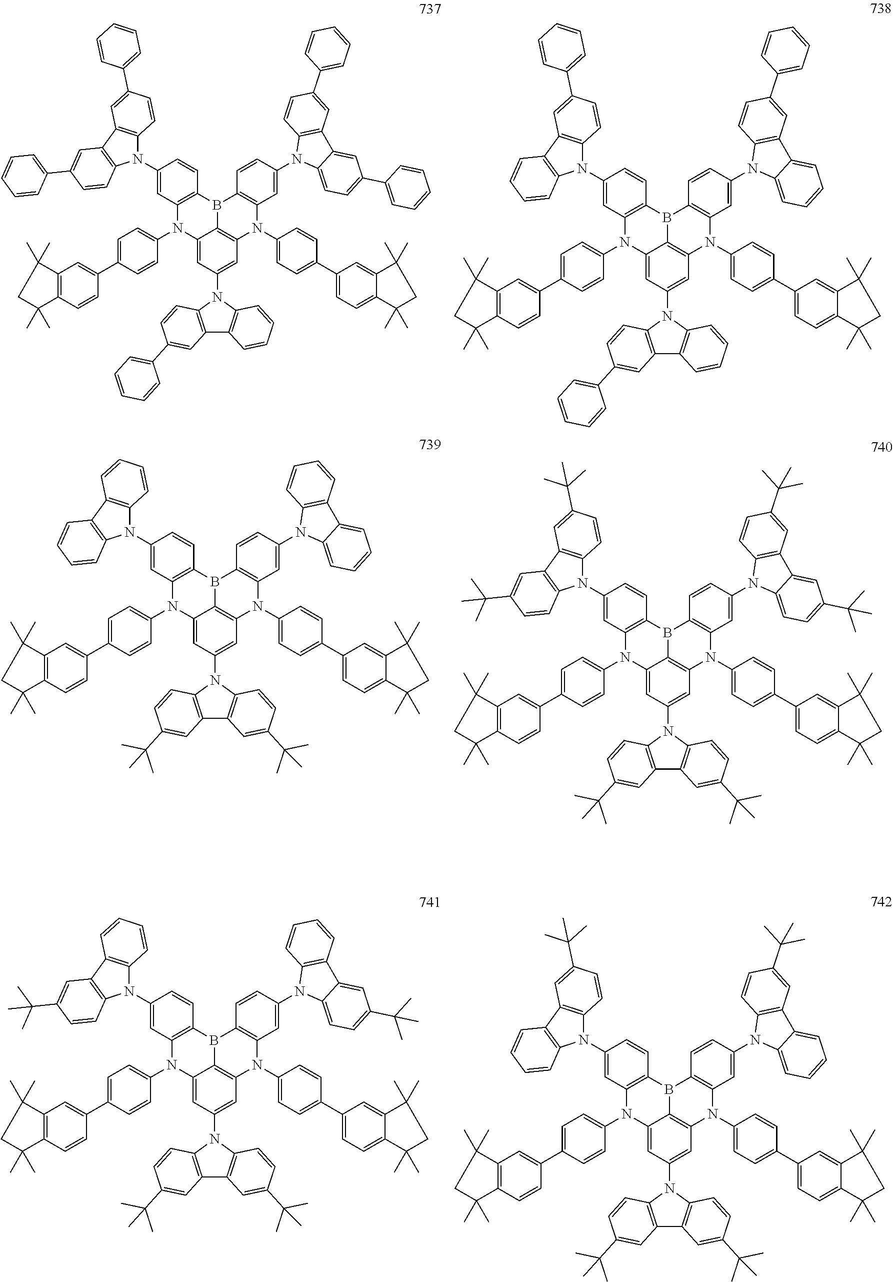

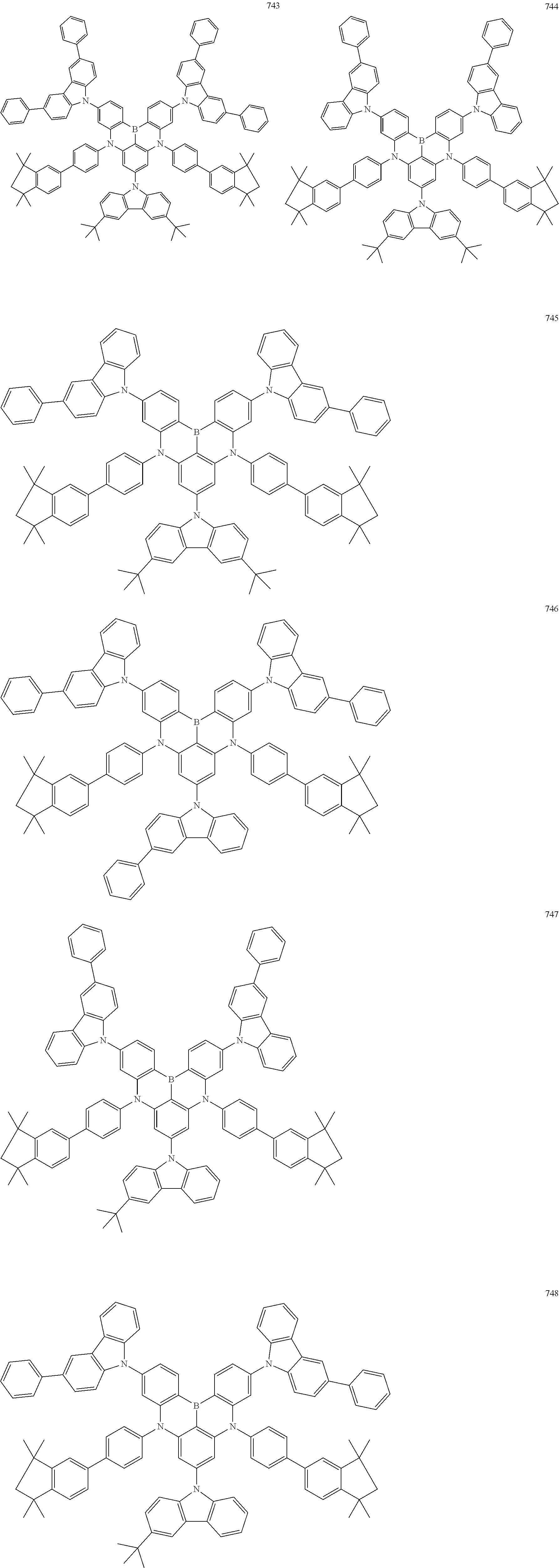

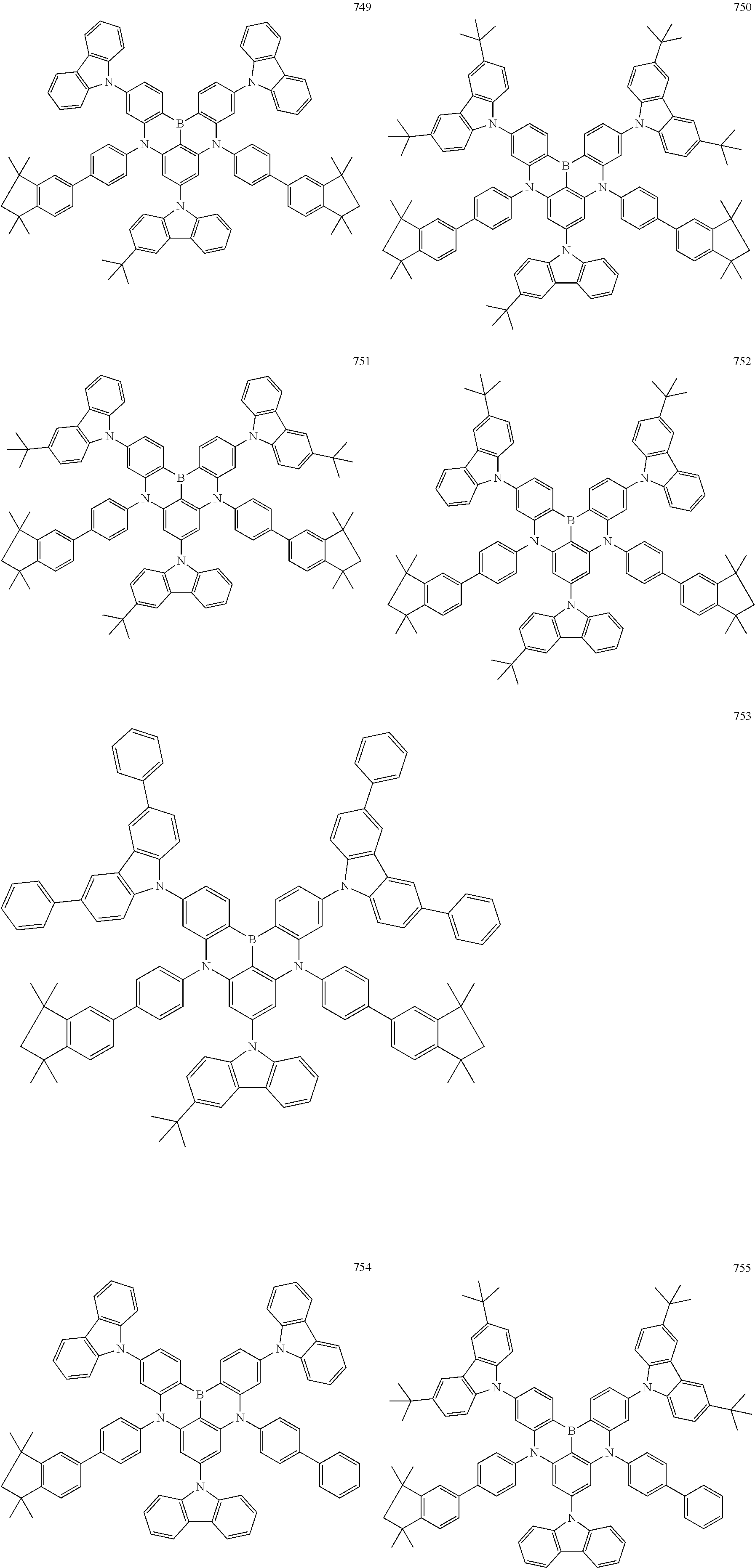

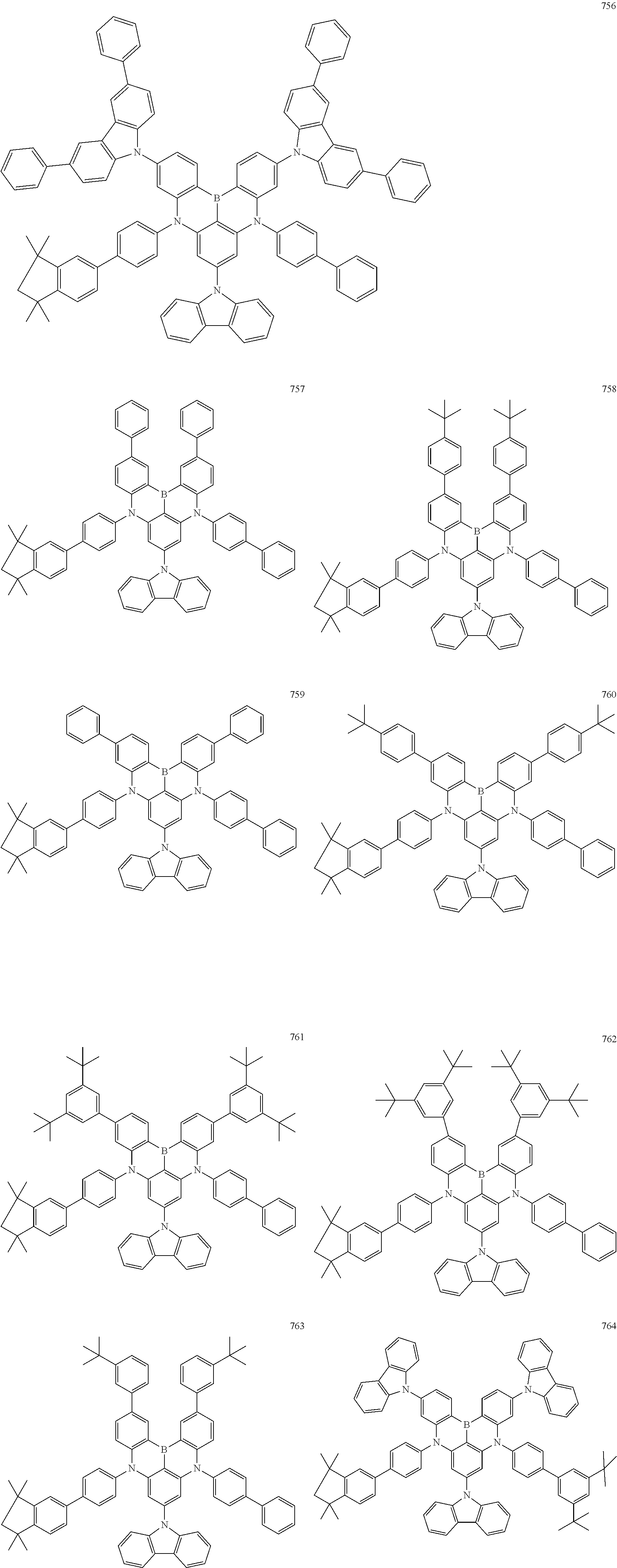

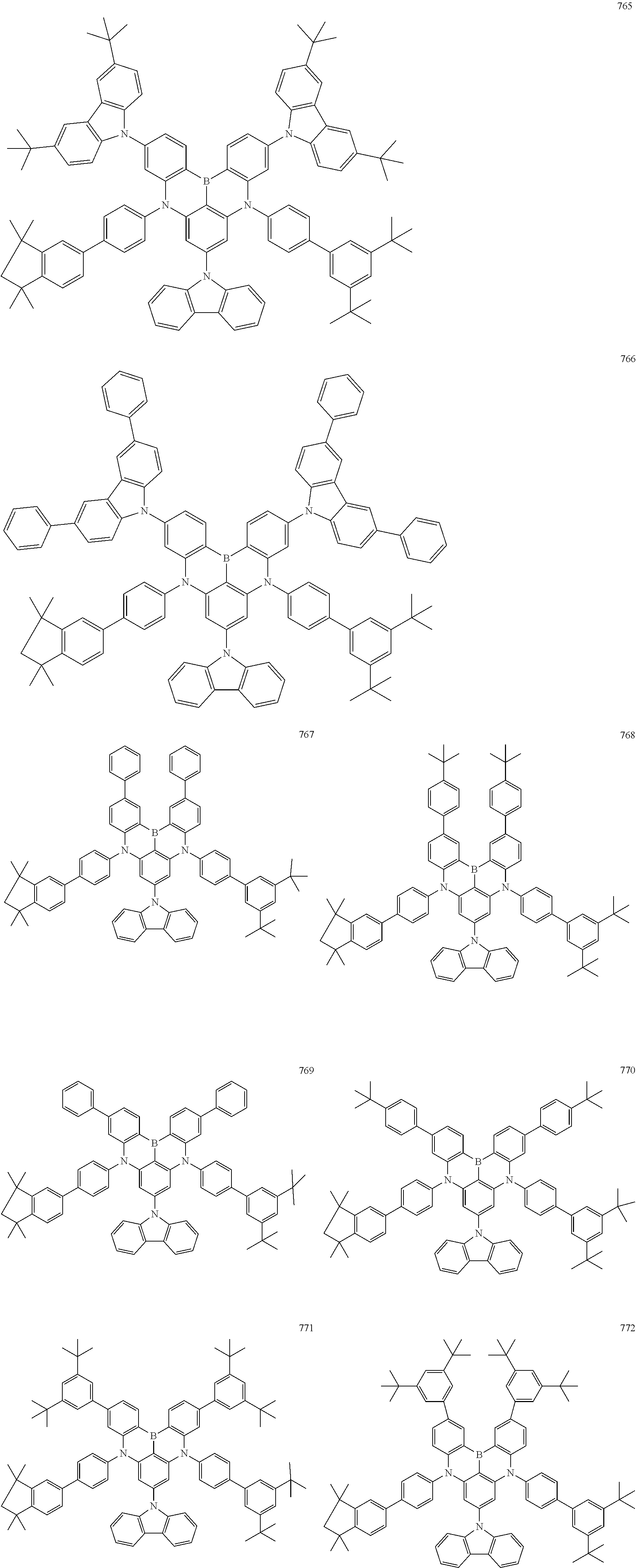

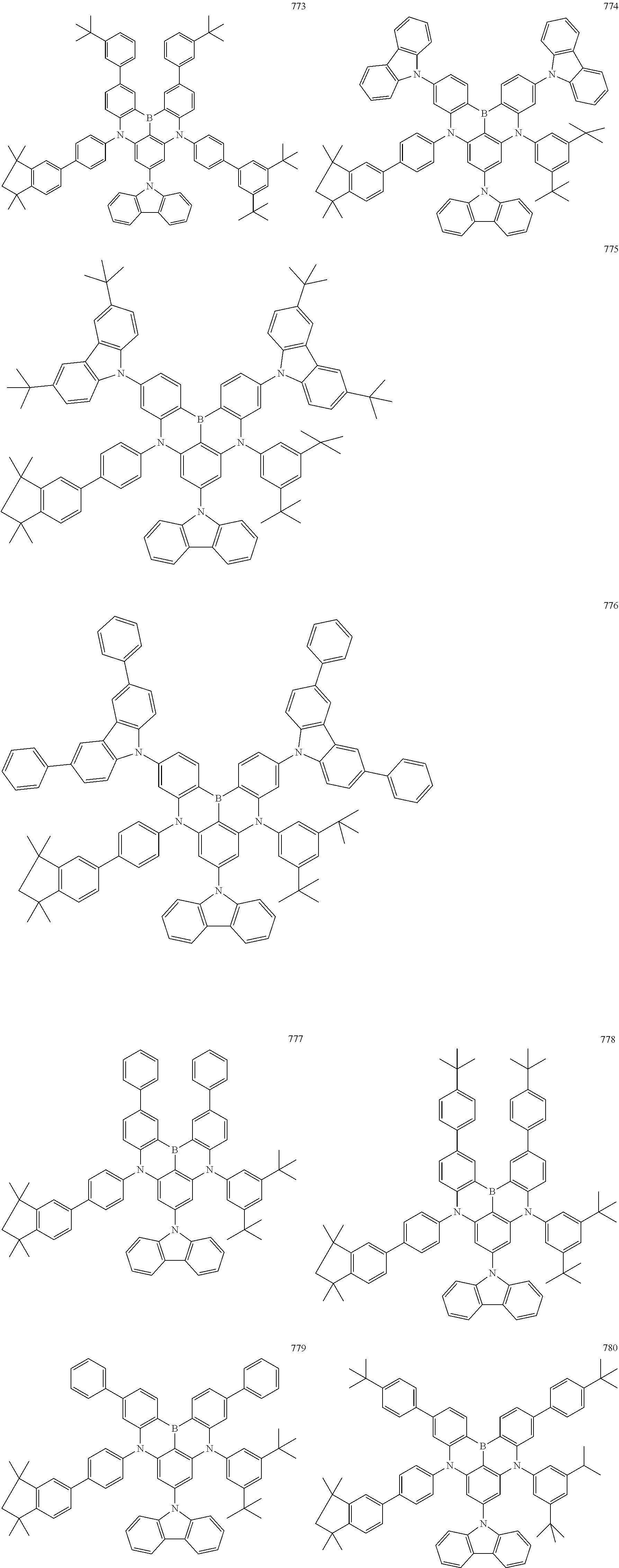

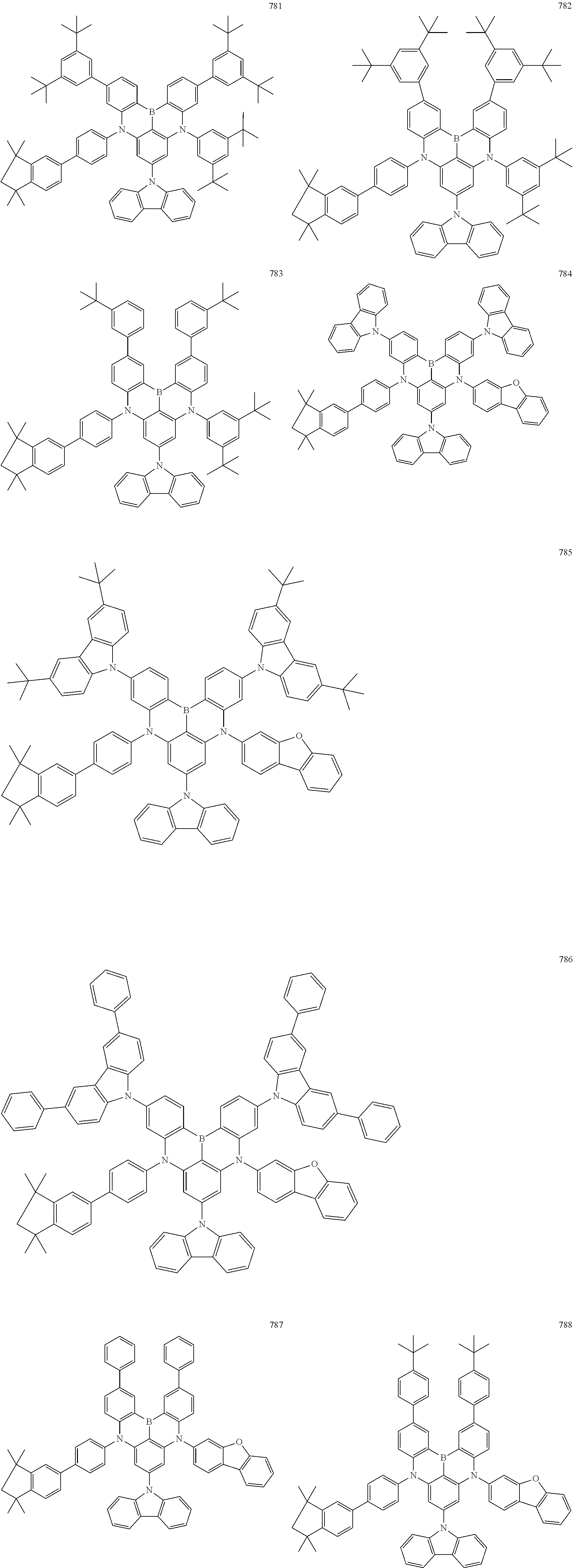

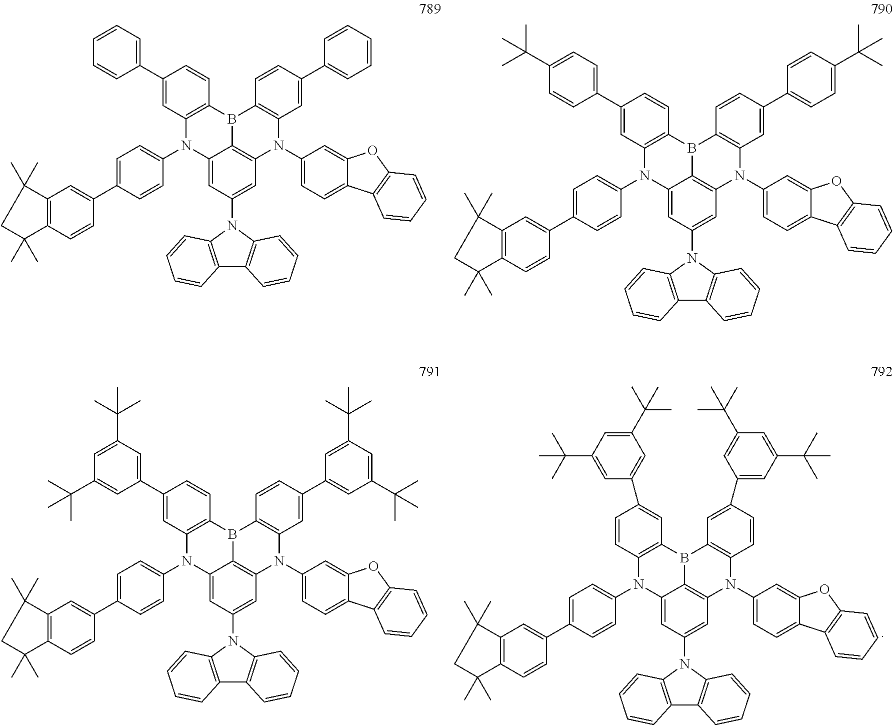

13. An organic light-emitting device comprising:
a first electrode;
a second electrode; and
an organic layer arranged between the first electrode and the second electrode and comprising an emission layer,
wherein the organic layer comprises the heterocyclic compound of claim 1.

14. The organic light-emitting device of claim 13, wherein the emission layer comprises the heterocyclic compound.

15. The organic light-emitting device of claim 14, wherein the emission layer comprises a host and an emitter, and
the emitter comprises the heterocyclic compound.

16. The organic light-emitting device of claim 15, wherein an amount of the host is greater than an amount of the heterocyclic compound.

17. The organic light-emitting device of claim 15, wherein the emission layer further comprises a sensitizer.

18. The organic light-emitting device of claim 17, wherein the sensitizer comprises a phosphorescent compound, a delayed fluorescence compound, or any combination thereof.

19. The organic light-emitting device of claim 14, wherein the emission layer emits blue light having a maximum emission wavelength of about 400 nm to about 490 nm.

20. A diagnostic composition comprising the heterocyclic compound of claim 1.
